(19) United States
(12) Patent Application Publication
(10) Pub. No.: US 2026/0022125 A1
(43) Pub. Date: Jan. 22, 2026

(54) PYRROLOPYRIDAZINE VEGFR INHIBITORS

(71) Applicant: Merck Sharp & Dohme LLC, Rahway, NJ (US)

(72) Inventors: Michael D. Altman, Needham, MA (US); Yejin Chang, Brookline, MA (US); Duane DeMong, Hanover, MA (US); Robert P. Hayes, Melrose, MA (US); Mitchell H. Keylor, Mansfield, MA (US); Chi Chip Le, Boston, MA (US); Umar Faruk Mansoor, Hopkinton, MA (US); Shasha Feng, Cambridge, MA (US); Kevin Anton Spiekermann, Bellevue, WA (US)

(73) Assignee: Merck Sharp & Dohme LLC, Rahway, NJ (US)

(21) Appl. No.: 19/271,987

(22) Filed: Jul. 17, 2025

Related U.S. Application Data
(60) Provisional application No. 63/673,421, filed on Jul. 19, 2024.

(51) Int. Cl. C07D 487/04; A61K 31/5025; A61K 31/5377; A61K 31/538; A61K 31/5386; A61K 31/55; A61K 31/553; C07D 519/00

(52) U.S. Cl. CPC C07D 487/04; A61K 31/5025; A61K 31/5377; A61K 31/538; A61K 31/5386; A61K 31/55; A61K 31/553; C07D 519/00

(57) ABSTRACT

Provided are compounds of formula I

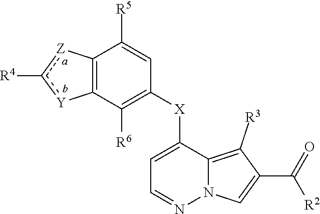

and the pharmaceutically acceptable salts, esters, and prodrugs thereof, which are VEGFR inhibitors. Also provided are methods of making compounds of Formula I, pharmaceutical compositions comprising compounds of Formula I, and methods of using these compounds to treat cancer and related diseases and conditions, comprising administering a compound of Formula I to a patient in need thereof.

This application claims the benefit of U.S. Provisional Application No. 63/673,421, filed Jul. 19, 2024, which is incorporated herein by reference in its entirety.

### BACKGROUND

Protein kinases are a family of enzymes that catalyze phosphorylation of the hydroxyl group of specific tyrosine, serine, or threonine residues in proteins. Typically, such phosphorylation dramatically perturbs the function of the protein, and thus protein kinases are pivotal in the regulation of a wide variety of cellular processes, including metabolism, cell proliferation, cell differentiation, and cell survival. Of the many different cellular functions in which the activity of protein kinases is known to be required, some processes represent attractive targets for therapeutic intervention for certain disease states. Two examples are angiogenesis and cell-cycle control, in which protein kinases play a pivotal role; these processes are essential for the growth of solid tumors as well as for other diseases.

### SUMMARY

The present disclosure is directed to compounds of structural Formula I:

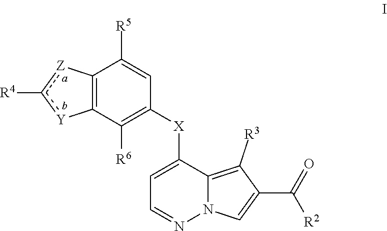

as well as pharmaceutically acceptable salts, esters, and prodrugs thereof, which are VEGFR modulators. Also provided are methods of making compounds of Formula I, pharmaceutical compositions comprising compounds of Formula I, methods of using these compounds for the potential prevention or treatment of a VEGFR-associated disease or disorder and the use of compounds of Formula I for the potential prevention or treatment of a VEGFR-associated disease or disorder.

### DETAILED DESCRIPTION

The present disclosure is directed to compounds of structural Formula I,

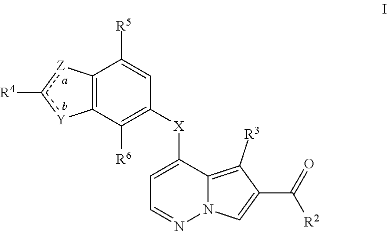

or a pharmaceutically acceptable salt thereof wherein
bonds “a” and “b” represent double bonds “- - - ” wherein: “a” and “b” are single bonds; or “a” is a single bond and “b” is a double bond; or “a” is a double bond and “b” is a single bond;
X is selected from —O—, —NH—, —N—C₁₋₆alkyl-, and —S—;
Y is selected from —O—, —S—, —N—, —C—R¹—, —C(H)—R¹—, —NR¹— and —CH₂—;
Z is selected from —C—R¹, —C(H)—R¹, —NR¹—, —S—, —N— and —O—;
when present, R¹ is selected from:
   (1) —C₁₋₆alkyl,
   (2) halo,
   (3) —C₀₋₆alkyl-C(O)NHR^{a},
   (4) —NHC(O)NHR^{a},
   (5) —NHC(O)R^{a},
   (6) —C₀₋₆alkyl-OR^{a},
   (7) —C₀₋₆alkyl-C(O)OR^{a},
   (8) —C₀₋₆alkyl-C(O)R^{a},
   (9) —C₀₋₆alkyl-cyano,
   (10) —C₀₋₆alkyl-NHR^{a},
   (11) —C₀₋₆alkyl-aryl, wherein the aryl is unsubstituted or substituted with 1-3 substituents independently selected from R^{a},
   (12) —C₀₋₆alkyl-(5-6 membered heteroaryl) wherein the heteroaryl has 1-3 ring heteroatoms independently selected from N, O, and S, and is unsubstituted or substituted with 1-3 substituents independently selected from R^{a},
   (13) —C₃₋₆cycloalkyl, unsubstituted or substituted with 1-3 substituents selected from R^{a},
   (14) —C₀₋₆alkyl-C(O)NR⁷R⁸,
   (15) —C₀₋₆alkyl-S(O)₂NHR^{a}, and
   (16) hydrogen;
when present, R^{a} is selected from:
   (1) hydrogen,
   (2) —C₁₋₆alkyl, unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, and —C₁₋₆haloalkyl,
   (3) —C₀₋₆alkyl-C₃₋₆cycloalkyl, unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, —C(O)—C₁₋₆alkyl, and —C₁₋₆haloalkyl,
   (4) —C₀₋₆alkyl-aryl, unsubstituted or substituted with 1-3 substituents independently selected from halo, ═O, —NH-cyclopropyl, —OH, —C₀₋₆alkyl-NR⁷R⁸, —C₀₋₆alkyl-cyano, —C₁₋₆alkyl, —O—C₀₋₆alkyl, and —C₁₋₆haloalkyl,
   (5) —O—C₁₋₆alkyl,
   (6) —C₀₋₆alkyl-(5-6 membered monocyclic heteroaryl), wherein the 5-6 membered monocyclic heteroaryl has 1-3 ring heteroatoms independently selected from N, O, and S, and is unsubstituted or substituted with 1-3 substituents independently selected from halo, ═O, —NH-cyclopropyl, —OH, —C₀₋₆alkyl-NR⁷R⁸, —C₀₋₆alkyl-cyano, —C₁₋₆alkyl, —O—C₀₋₆alkyl, and —C₁₋₆haloalkyl,
   (7) —C₁₋₆alkyl-S—C₁₋₆alkyl,
   (8) —C₃₋₆ monocyclic heterocyclyl having 1-3 ring heteroatoms independently selected from N, O, and S, unsubstituted or substituted with 1-3 substituents independently selected from halo, —OH, —C₀₋₆alkyl-NR⁷R⁸, —C₀₋₆alkyl-cyano, —C₁₋₆alkyl, —OC₀₋₆alkyl, —C₁₋₆haloalkyl, and =0,
   (9) 5-10 membered spiro bicyclic or tricyclic cycloalkyl, unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, —C₁₋₆haloalkyl, and ═O,
   (10) 5-10 membered spiro bicyclic or tricyclic heterocyclyl having 1-3 ring heteroatoms independently selected from N, O, and S unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, —C₁₋₆haloalkyl, and ═O,
   (11) 5-6 membered bridged bicyclic cycloalkyl, unsubstituted or substituted with 1 substituent selected from —OH, halo and haloalkyl,
   (12) halo,
   (13) —C(O)O—C₁₋₆alkyl, and
   (14) —NH₂;
when present, each of R⁷ and R⁸ is independently selected from
   (1) hydrogen,
   (2) —C₀₋₆alkyl-(3-6 membered heterocyclyl) wherein the 3-6 membered heterocyclyl has 1-3 ring heteroatoms selected from N, O, and S, and is unsubstituted or substituted with 1-3 substituents independently selected from R^{b},
   (3) —C₁₋₆alkyl, unsubstituted or substituted with 1-3 substituents independently selected from R^{b},
   (4) —C₁₋₆ alkyl-O—C₁₋₆alkyl, unsubstituted or substituted with 1-3 substituents independently selected from R^{b},
   (5) —C₀₋₆alkyl-C₃₋₆cycloalkyl, wherein the cycloalkyl is unsubstituted or substituted with 1-3 substituents independently selected from R^{b}, and
   (6) 5-10 membered bicyclic or tricyclic cycloalkyl, wherein the cycloalkyl is unsubstituted or substituted with 1-3 substituents independently selected from R^{b};
when present, R^{b} is selected from
   (1) —C₁₋₆alkyl,
   (2) halo,
   (3) —NH₂,
   (4) ═O, and
   (5) —OH;
or alternatively, R⁷ and R⁸ together with the nitrogen to which they are attached form 3-10 membered heterocyclyl having 0-3 ring heteroatoms independently selected from N, O, and S, in addition to the nitrogen to which R⁷ and R⁸ are attached, wherein the heterocyclyl is optionally substituted with 1-4 substituents independently selected from R^{c};
when present, R^{c} is selected from
   (1) —C₀₋₆alkyl-OH,
   (2) —C₁₋₆alkyl, unsubstituted or substituted with 1-3 substituents independently selected from —OH,
   (3) —C₀₋₆alkyl-cyano,
   (4) —C₀₋₆alkyl-O—C₁₋₃alkyl,
   (5) —C₀₋₃alkyl-(5-6 membered monocyclic heteroaryl) wherein the 5-6 membered heterocyclyl has 1-3 ring heteroatoms selected from N, O, and S, unsubstituted or substituted with 1-3 substituents independently selected from C₁₋₆alkyl,
   (6) halo,
   (7) —C₁₋₆haloalkyl,
   (8) C₀₋₆alkyl-aryl, wherein the aryl is unsubstituted or substituted with 1-3 substituents independently selected from C₁₋₆haloalkyl, and halo,
   (9) —C₃₋₆cycloalkyl,
   (10) —C₀₋₆alkyl-N, unsubstituted or substituted with 1-3 substituents independently selected from hydrogen and C₁₋₆alkyl,
   (11) —NH—C(O)C₁₋₆alkyl,
   (12) —C₀₋₃alkyl-(3-6 membered monocyclic heterocyclyl) wherein the 3-6 membered monocyclic heterocyclyl has 1-3 ring heteroatoms selected from N, O, and S, and is unsubstituted or substituted with 1 ═O substituent,
   (13) —O—C₁₋₆haloalkyl,
   (14) ═O,
   (15) hydrogen, and
   (16) —C(O)OC₁₋₆alkyl;
R² is selected from
   (1) —O—C₁₋₆alkyl, and
   (2)

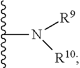

each of R⁹ and R¹⁰ is independently selected from
   (1) hydrogen,
   (2) —C₀₋₆alkyl-(3-6 membered heterocyclyl) wherein the 3-6 membered heterocyclyl has 1-3 ring heteroatoms selected from N, O, and S, and is unsubstituted or substituted with 1-3 substituents independently selected from R^{d},
   (3) —C₁₋₆alkyl, unsubstituted or substituted with 1-3 substituents independently selected from R^{d},
   (4) —C₁₋₆alkyl-O—C₁₋₆alkyl, unsubstituted or substituted with 1-3 substituents independently selected from R^{d},
   (5) —C₁₋₆alkyl-C₃₋₆cycloalkyl, wherein the cycloalkyl is unsubstituted or substituted with 1-3 substituents independently selected from R^{d}, and
   (6) 5-10 membered bicyclic or tricyclic cycloalkyl, wherein the cycloalkyl is unsubstituted or substituted with 1-3 substituents independently selected from R^{d};
when present, R^{d} is selected from
   (1) —C₁₋₆alkyl,
   (2) halo,
   (3) —NH₂,
   (4) ═O, and
   (5) —OH;
or alternatively, R⁹ and R¹⁰ together with the nitrogen to which they are attached form 4-10 membered heterocyclyl having 0-3 ring heteroatoms independently selected from N, O, and S, in addition to the nitrogen to which R⁹ and R¹⁰ are attached, wherein the heterocyclyl is optionally substituted with 1-4 substituents independently selected from R^{e}; when present, R^{e} is selected from
   (1) —C₀₋₆alkyl-OH,
   (2) —C₁₋₆alkyl, unsubstituted or substituted with 1-3 substituents selected from —OH,
   (3) —C₀₋₆alkyl-cyano,
   (4) —C₀₋₆alkyl-O—C₁₋₃alkyl,
   (5) —C₀₋₃alkyl-(5-6 membered monocyclic heteroaryl) wherein the heteroaryl has 1-3 ring heteroatoms selected from N, O, and S, and is unsubstituted or substituted with 1-3 substituents selected from C₁₋₆alkyl,
   (6) halo,
   (7) —C₁₋₆haloalkyl,
   (8) C₀₋₆alkyl-aryl, wherein the aryl is unsubstituted or substituted with 1-3 substituents independently selected from C₁₋₆haloalkyl, and halo,
   (9) —C₃₋₆cycloalkyl,
   (10) —C₀₋₆alkyl-N(Rf)₂, wherein each Rf is independently selected from independently selected from hydrogen and C₁₋₆alkyl,
   (11) —NH—C(O)C₁₋₆alkyl,
   (12) —C₀₋₃alkyl-(3-6 membered monocyclic heterocyclyl) wherein the heterocyclyl has 1-3 ring heteroatoms selected from N, O, and S, and is unsubstituted or substituted with 1 ═O substituent,
   (13) —O—C₁₋₆haloalkyl,
   (14) ═O,
   (15) hydrogen, and
   (16) —C(O)OC₁₋₆alkyl;
R³ is selected from:
   (1) hydrogen,
   (2) —C₁₋₆alkyl,
   (3) —C₃₋₆cycloalkyl,
   (4) —C₁₋₆haloalkyl,
   (5) —C₁₋₆alkyl-O—C₁₋₆alkyl, and
   (6) —C₁₋₆alkyl-OH;
R⁴ is selected from:
   (1) hydrogen,
   (2) halogen,
   (3) —C₁₋₆alkyl,
   (4) —C(O)NH—C₀₋₆alkyl,
   (5) —C₀₋₆alkyl-(O)OC₁₋₆alkyl,
   (6) —NH₂
   (7) —C₁₋₆haloalkyl,
   (8) —C(O)NH₂, and
   (9) —C₃₋₆cycloalkyl;
R⁵ is selected from:
   (1) hydrogen,
   (2) halogen,
   (3) —C₁₋₆alkyl, and
   (4) C₁₋₆alkyl-(O)OC₁₋₆alkyl;
R⁶ is selected from:
   (1) hydrogen,
   (2) halogen, and
   (3) —C₁₋₆alkyl.

In Embodiment 2 of this disclosure are compounds of Formula I, or a pharmaceutically acceptable salt of any of the foregoing, wherein R¹ is
(1) —C₁₋₅alkyl,
(2) —C₀₋₃alkyl-C(O)NH—C₀₋₃alkyl, unsubstituted or substituted with 1-3 substituents independently selected from: cyclobutanyl, unsubstituted or substituted with 1-3 substituents independently selected from halo, —C₁₋₃alkyl-haloalkyl, —C₁₋₃alkyl, —O—C₁₋₃ alkyl, —OH, and C₀₋₁alkyl-cyano; bicyclo[1.1.1]pentanyl, unsubstituted or substituted with 1-3 substituents independently selected from C₁₋₃haloalkyl, —OH and —F; cyclopropanyl, unsubstituted or substituted with 1-3 substituents independently selected from —C₀₋₁alkyl-cyano, —C₁₋₃alkyl, —C₁₋₃haloalkyl, and —F; phenyl unsubstituted or substituted with 1-3 substituents independently selected from methyl or —F; cyclopentanyl, unsubstituted or substituted with 1-3 substituents independently selected from —F; spiro[3.3]heptanyl; spiro[2.3]hexanyl, unsubstituted or substituted with 1 —F substituent; oxaspiro[3.3]heptanyl; thietanyl, substituted with 1 ═O substituent; azetidinyl, unsubstituted or substituted with 1 —C₁₋₃haloalkyl substituent; tetrahydrofuranyl, unsubstituted or substituted with 1 —C₁₋₃alkyl substituent; azetidinyl, substituted with 1 —C₁₋₃haloalkyl substituent; —OH; —C₀₋₃alkyl-C(O)NH₂; and oxetanyl, unsubstituted or substituted with 1 methyl substituent,
(3) —C₀₋₃alkyl-C(O)O—C₁₋₃alkyl,
(4) —C₀₋₃alkyl-C(O)-heterocyclyl, wherein the heterocyclyl is unsubstituted or substituted with 1 halo substituent,
(5) —SO₂NH—C₃₋₆cycloalkyl, or —SO₂NH—C₁₋₃alkyl, or
(6) hydrogen.

In Embodiment 3 of this disclosure are compounds of Formula I, or Embodiments 1 or 2, or a pharmaceutically acceptable salt of any of the foregoing, wherein R¹ is (1) —C₁₋₃alkyl,
(2) —C(O)NH—C₀₋₁alkyl-cyclobutanyl, unsubstituted or substituted with 1-2 substituents independently selected from —F, —CF₃, C₁₋₃alkyl, —OCH₃, —OH and C₀₋₁alkyl-cyano; —C(O)NH-bicyclo[1.1.1]pentanyl, unsubstituted or substituted with 1 substituent selected from —OH, —F and —CF₃; —C(O)NH—C₀₋₁alkyl-cyclopropanyl, unsubstituted or substituted with 1-2 —F substituents; —C(O)NH—C₀₋₁alkyl-phenyl; —C(O)NH—C₁₋₃alkyl, unsubstituted or substituted with 1 —OH substituent; —C(O)NH—C₀₋₁alkyl-cyclopentanyl; —C(O)NH-spiro[3.3]heptanyl; —C(O)NH-spiro[2.3]hexanyl, unsubstituted or substituted with 1 —F substituent; —C(O)NH-oxaspiro[3.3]heptanyl; —C(O)NH-thietane, substituted with 1 ═O substituent; tetrahydrofuranyl, unsubstituted or substituted with 1 methyl substituent; azetidinyl, substituted with 1 —CH₂CF₃ substituent; or —C(O)NH-oxetanyl, unsubstituted or substituted with 1 methyl substituent,
(3) —C(O)OCH₃ or —C(O)OCH₂CH₃,
(4) —C(O)-azetidinyl, unsubstituted or substituted with one —F substituent,
(5) —SO₂NH-cyclopropyl, or —SO₂NHCH₃, or
(6) hydrogen.

In Embodiment 4 of this disclosure are compounds of Formula I, or Embodiments 1 to 3, or a pharmaceutically acceptable salt of any of the foregoing, wherein R¹ is —H, —C₁₋₃alkyl,

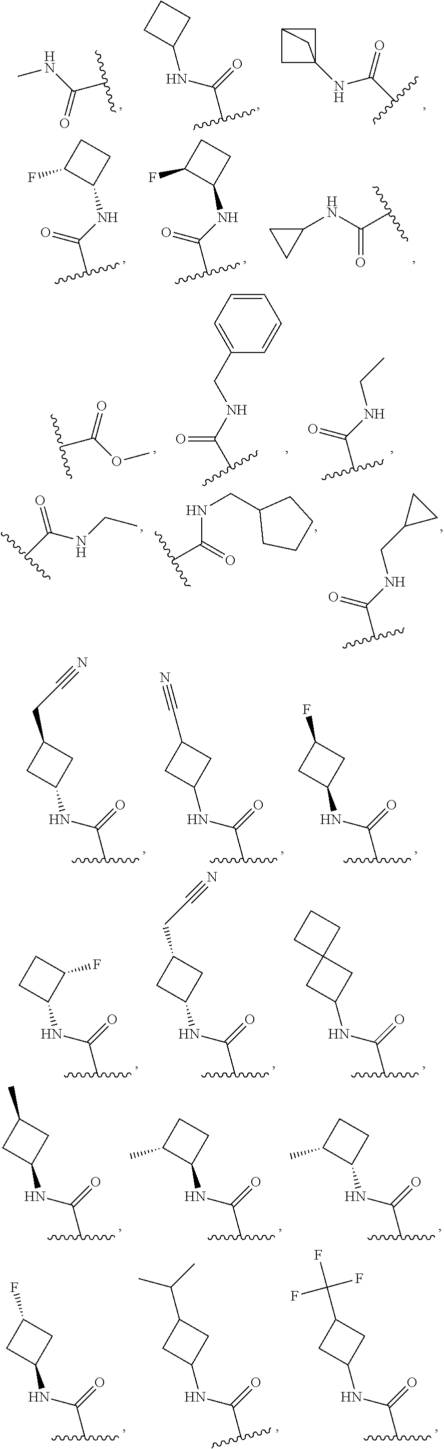

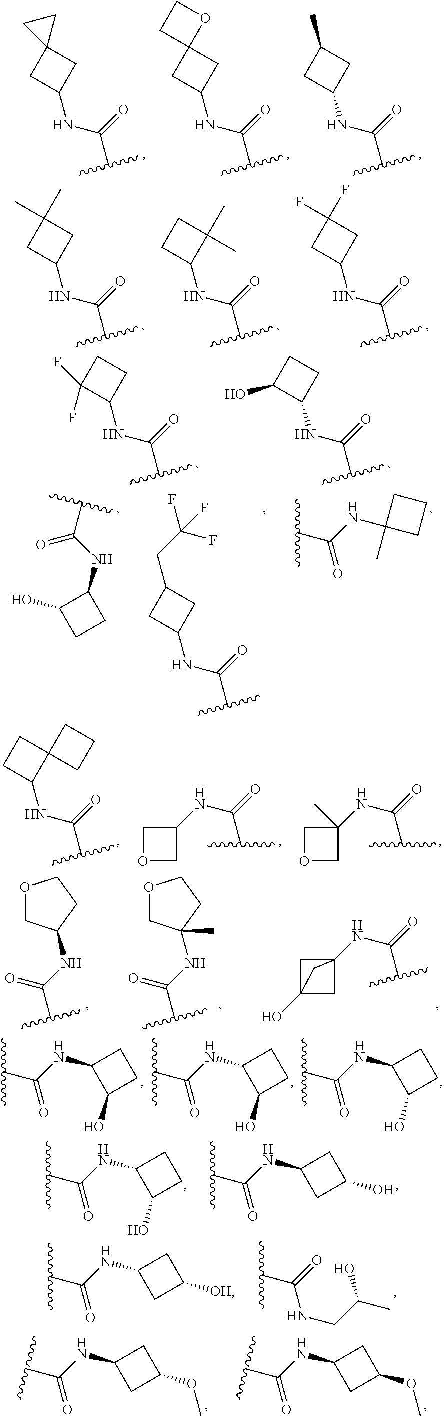

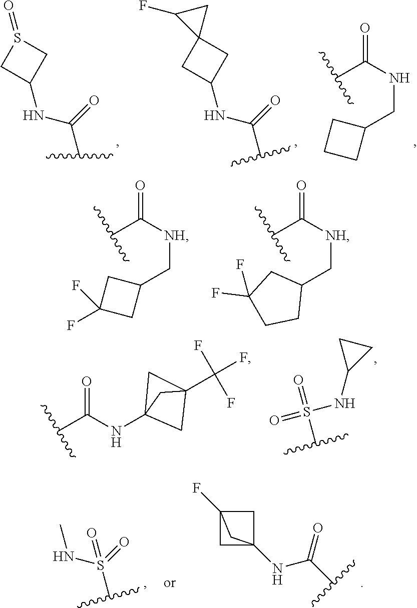

In Embodiment 5 of this disclosure are compounds of Formula I, or Embodiments 1 to 4, or a pharmaceutically acceptable salt of any of the foregoing, wherein R^{a} is
(1) hydrogen,
(2) —C₁₋₃alkyl,
(3) —C₀₋₃alkyl-C₃₋₆cycloalkyl, unsubstituted or substituted with 1-3 substituents independently selected from halo, —C₁₋₂alkyl-cyano, —C₁₋₆alkyl, —C₁₋₆alkyl-O—C₀₋₆alkyl, and —C₁₋₆haloalkyl,
(4) —C₀₋₆alkyl-aryl, unsubstituted or substituted with 1-3 substituents independently selected from halo, ═O, and —NH—C₃₋₆cycloalkyl,
(5) —O—C₁₋₆alkyl,
(6) 5-6 membered monocyclic heteroaryl having 1-3 ring heteroatoms independently selected from N and S, unsubstituted or substituted with 1-3 —C₁₋₆alkyl substituents,
(7) —C₁₋₃alkyl-S—C₁₋₃alkyl,
(8) —C₃₋₆ monocyclic heterocyclyl having 1 ring heteroatom selected from N and S, unsubstituted or substituted with 1-3 substituents independently selected from halo and ═O,
(9) 5-10 membered spiro bicyclic cycloalkyl, unsubstituted or substituted with 1-3 substituents selected from halo,
(10) 5-10 membered spiro bicyclic heterocyclyl having 1 ring heteroatom selected from N, O, and S,
(11) 5-6 membered bridged bicyclic cycloalkyl, unsubstituted or substituted with 1 substituent selected from halo and C₁₋₃haloalkyl,
(12) halo,
(13) —C(O)O—C₁₋₃alkyl, or
(14) —NH₂.

In Embodiment 6 of this disclosure are compounds of Formula I, or Embodiments 1 to 5, or a pharmaceutically acceptable salt of any of the foregoing, wherein R^{a} is
(1) hydrogen,
(2) methyl,
(3) —C₀₋₁alkyl-cyclobutanyl, unsubstituted or substituted with 1-2 substituents independently selected from —F, —C₁₋₃alkyl, —CF₃—C(O)CH₃, and —C₀₋₁alkyl-cyano; —C₀₋₂alkyl-cyclopropanyl, unsubstituted or substituted with 1-2 —F substituents; or —C₀₋₂alkyl-cyclopentyl, unsubstituted or substituted with 2 —F substituents,
(4) —C₀₋₁alkyl-phenyl, unsubstituted or substituted with 1 —F substituent; or cyclobutenyl, substituted with 2 ═O substituents and 1 —NH-cyclopropyl substituent,
(5) —OCH₃,
(6) thiophenyl, unsubstituted or substituted with 1 methyl substituent; or thiazolyl, substituted with 1 methyl substituent,
(7) —CH₂SCH₃,
(8) azetidinyl, substituted with 1 —F substituent; or thietanyl, substituted with 1 ═O substituent,
(9) spiro[3.3]heptanyl; spiro[2.3]hexanyl, unsubstituted or substituted with 1 —F substituent,
(10) oxaspiro[3.3]heptanyl,
(11) bicyclo[1.1.1]pentane, unsubstituted or substituted with 1 substituent selected from —F and —CF₃,
(12) —F or —Cl,
(13) —C(O)OCH₃, or
(14) —NH₂.

In Embodiment 7 of the disclosure are compounds of Formula I, or Embodiments 1 to 6, or a pharmaceutically acceptable salt of any of the foregoing, wherein R² is
(1) —OC₁₋₂alkyl,
(2) —NH—C₀₋₃alkyl-3-6 membered heterocyclyl wherein the heterocyclyl has 1 ring heteroatom selected from S and O, and is unsubstituted or substituted with 1-3 substituents independently selected from —C₁₋₆alkyl, halo, ═O, and —OH,
(3) —NH—C₁₋₅alkyl, unsubstituted or substituted with 1-3 substituents independently selected from —C₁₋₆alkyl, halo, ═O, and —OH,
(4) —NH—C₀₋₃alkyl-C₃₋₆cycloalkyl, unsubstituted or substituted with 1-3 substituents independently selected from —C₁₋₆alkyl, halo, ═O, and —OH,
(5) 3-7 membered monocyclic heterocyclyl having 0-3 ring heteroatoms independently selected from N, O, and S, in addition to the nitrogen to which R⁹ and R¹⁰ are attached, wherein the monocyclic heterocyclyl is unsubstituted or substituted with 1-4 substituents independently selected from: —C₁₋₆alkyl, unsubstituted or substituted with 1-3 substituents selected from —OH; —NH₂; —C₀₋₆alkyl-cyano; —C₀₋₆alkyl-O—C₁₋₃alkyl; —C₀₋₃alkyl-5-6 membered monocyclic heteroaryl having 1-3 ring heteroatoms independently selected from N, O, and S, unsubstituted or substituted with 1-3 C₁₋₆alkyl substituents; halo; —OH; —C₁₋₆haloalkyl; C₀₋₆alkyl-aryl, wherein the aryl is unsubstituted or substituted with 1-3 substituents independently selected from C₁₋₆haloalkyl and halo; —C₃₋₆cycloalkyl; —C₀₋₆alkyl-N, unsubstituted or substituted with 1-3 substituents independently selected from hydrogen and —C₁₋₆alkyl; —NH—C(O)C₁₋₆alkyl; —N(CH₃)—C(O)C₁₋₆alkyl; and —C₀₋₃alkyl-3-6 membered monocyclic heterocyclyl having 1-3 ring heteroatoms independently selected from N, O, and S, unsubstituted or substituted with 1-3 ═O substituents,
(6) 5-10 membered spiro bicyclic heterocyclyl having 0-3 ring heteroatoms independently selected from N, O, and S, in addition to the nitrogen to which R⁹ and R¹⁰ are attached, wherein the spiro bicyclic heterocyclyl is unsubstituted or substituted with 1-3 substituents independently selected from cyano, ═O, —OH, and —C₁₋₆alkyl,
(7) 5-10 membered bridged heterocyclyl having 0-3 ring heteroatoms independently selected from N, O, and S, in addition to the nitrogen to which R⁹ and R¹⁰ are attached, wherein the bridged heterocyclyl is unsubstituted or substituted with 1-3 substituents independently selected from methyl, —C₀₋₆alkyl-OH, and hydrogen, or
(8) 5-10 membered fused bicyclic heterocyclyl having 0-3 ring heteroatoms independently selected from N, O, and S, in addition to the nitrogen to which R⁹ and R¹⁰ are attached, wherein the fused bicyclic heterocyclyl is unsubstituted or substituted with 1-3 substituents independently selected from halo, hydrogen, —C(O)OC₁₋₆alkyl, —O—C₁₋₆alkyl, —OH, and —O—C₁₋₆haloalkyl.

In Embodiment 8 of the disclosure are compounds of Formula I, or Embodiments 1 to 7, or a pharmaceutically acceptable salt of any of the foregoing, wherein R² is
(1) —OCH₃ or —OCH₂CH₃,
(2) —NH-thietanyl, substituted with 2 ═O substituents and 1 methyl substituent; —NH— tetrahydrothiopyranyl, substituted with 2 ═O substituents and 1 methyl substituent; or —N(CH₃)CH₂-pyrrolidinyl, wherein the pyrrolidinyl is substituted 2 substituents independently selected from methyl and ═O,
(3) —NH—CH₂C(CH₃)₂CH₂OCH₃,
(4) —NH-cyclohexnanyl, substituted with 1-3 substituents independently selected from —OH and —F; —N(CH₃)-cyclohexanyl substituted with 1 —OH substituent; or —NHCH(CH₃)-cyclopropanyl,
(5) pyrrolidinyl, unsubstituted or substituted with 1-3 substituents independently selected from —OH; pyrazolyl, unsubstituted or substituted with methyl; —CH₂OCH₃; —CH₂-cyano; —N(CH₃)C(O)CH₃; —CH₂-triazolyl, —CHF₂, pyridinyl; —C₁₋₄alkyl; C₀₋₁alkyl-phenyl, unsubstituted or substituted with 1 substituent selected from —Cl, —F —CF₃; —OCH₃; cyclopropyl; oxadiazolyl, substituted with 1 methyl substituent; imidazole, substituted with 1 methyl substituent; or —CH₂N(CH₃)₂,
(6) piperidinyl, substituted with 1-2 substituents independently selected from —OH, methyl, and ethyl,
(7) azetidinyl, unsubstituted or substituted with 1-3 substituents independently selected from —C₀₋₃alkyl-cyano, —OCH₃, pyrazole, —CH₂-triazole, —OH, methyl, ethyl, —CH(CH₃)₂, —CH₂OCH₃, —F, —CHF₂, —CF₃, —F, —C(CH₃)₂OH, —NH₂, cyclopropyl, —CH₂N(CH₃)₂, and —NHC(O)CH₃; or prrolidinyl, substituted with 1 ═O substituent,
(8) morpholinyl, substituted with 1 substituent selected from —CH(CH₃)₂ and methyl,
(9) azepanyl, unsubstituted or substituted with 1 —OH substituent,
(10) oxazepanyl,
(11) oxa-azaspiro[3.4]octanyl, unsubstituted or substituted with 1-2 substituents independently selected from methyl and —OH; diazaspiro[3.4]octanyl, substituted with 1 ═O substituent and 1 hydrogen substituent; azaspiro[3.3]heptanyl, substituted with 1 substituent selected from —OH and cyano; diazaspiro[4.4]nonanyl, substituted with 1 ═O substituent; oxa-azaspiro[4.4]nonanyl; oxa-azaspiro[3.5]nonanyl; oxa-azaspiro[4.5]decanyl, unsubstituted or substituted with 1 ═O substituent; oxadiazaspiro[3.4]octanyl, substituted with 1 ═O substituent; azaspiro[2.3]hexanyl; or oxa-azaspiro[bicyclo[3.1.0]hexane-pyrrolidinyl],
(12) azabicyclo[2.1.1]hexanyl, unsubstituted or substituted with 1-3 substituents independently selected from methyl, —CH₂OH, —OH, —C(CH₃)₂OH, and —OCH₃; azabicyclo[3.1.0]hexanyl, unsubstituted or substituted with 1 —C(O)OCH₂CH₃ substituent; oxa-azabicyclo[2.2.1]heptanyl; azabicyclo[3.1.1]heptanyl; azabicyclo[2.2.1]heptanyl, unsubstituted or substituted with 1-4 substituents independently selected from —OH, hydrogen, and methyl; oxaazabicyclo[3.2.0]heptanyl; or azabicyclo[3.2.0]heptanyl, substituted with 1-2 substituents independently selected from hydrogen, —OCH₂CF₃, —OCH₃ and —OH, or
(13) hexahydrocyclopenta[c]pyrrolyl, unsubstituted or substituted with 2 —F substituents; hexahydrofuro[3,2-b]pyrrolyl; octahydro-indolyl; hexahydropyrano[3,2-c]pyridinyl; hexahydrofuro[3,2-b]pyridinyl; or octahydrobenzo[b][1,4]oxazinyl, substituted with 1 methyl substituent.

In Embodiment 9 of the disclosure are compounds of Formula I, or Embodiments 1 to 8, or a pharmaceutically acceptable salt of any of the foregoing, wherein R² is

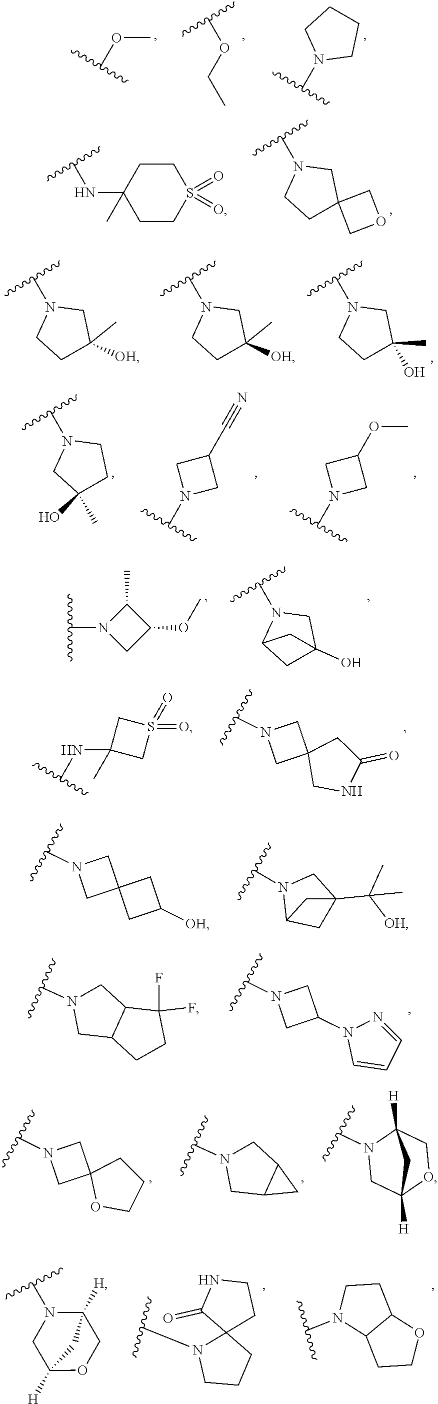

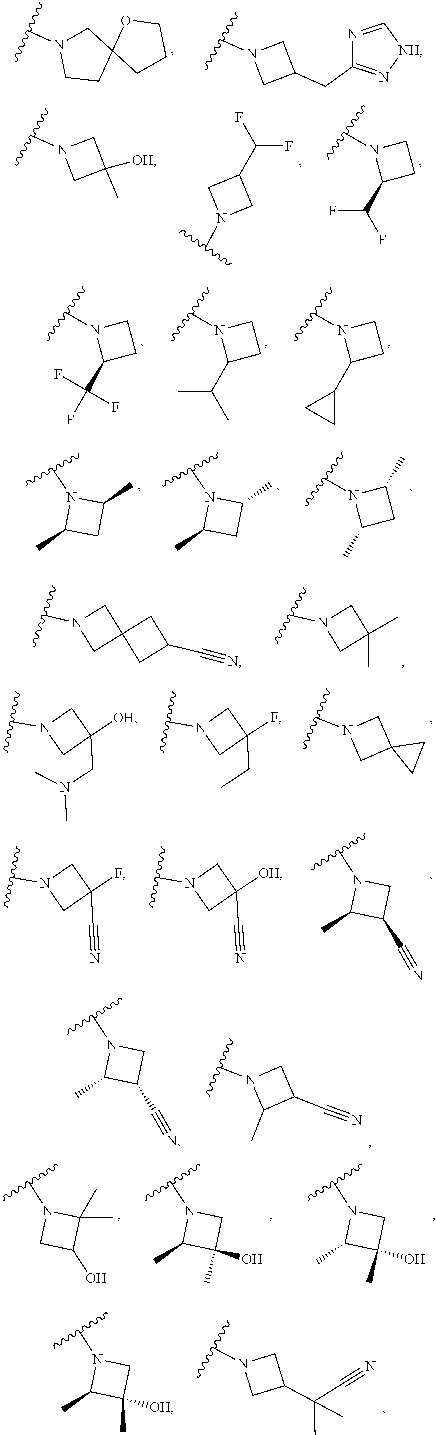

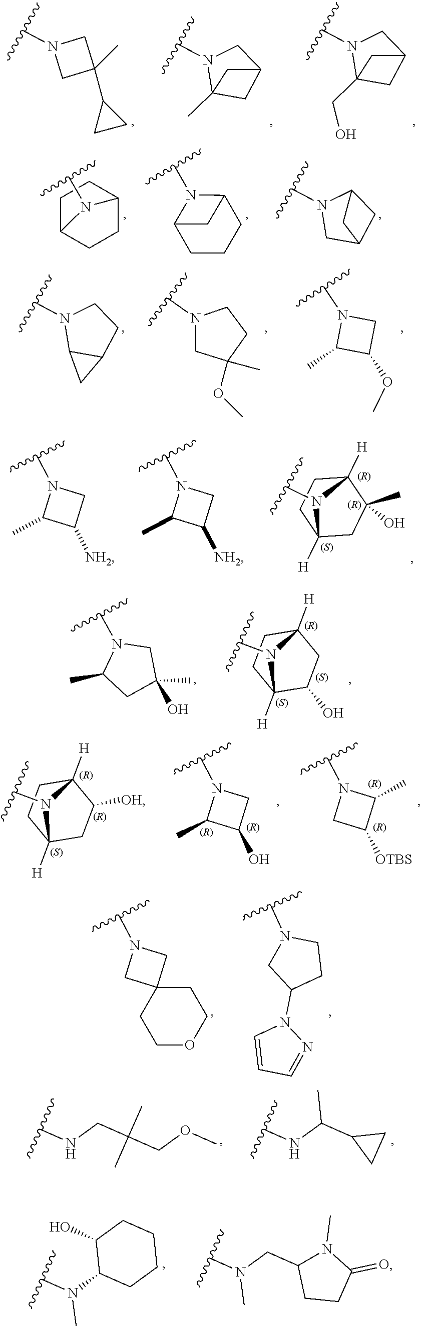

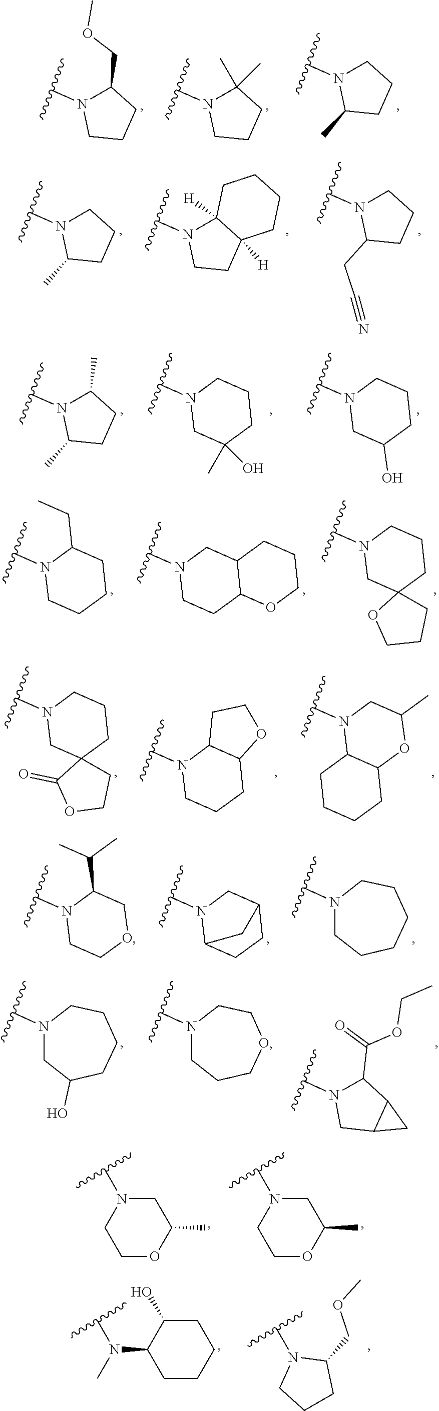

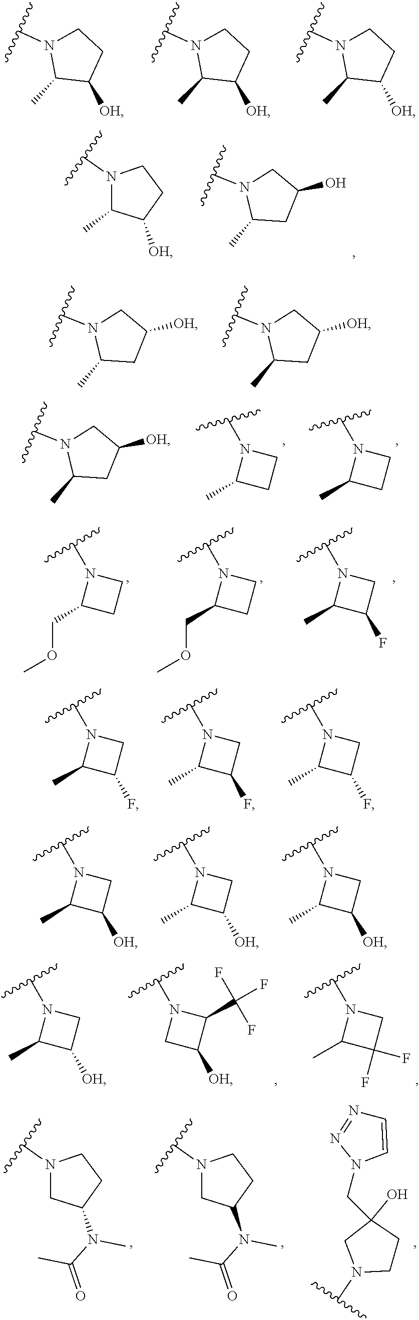

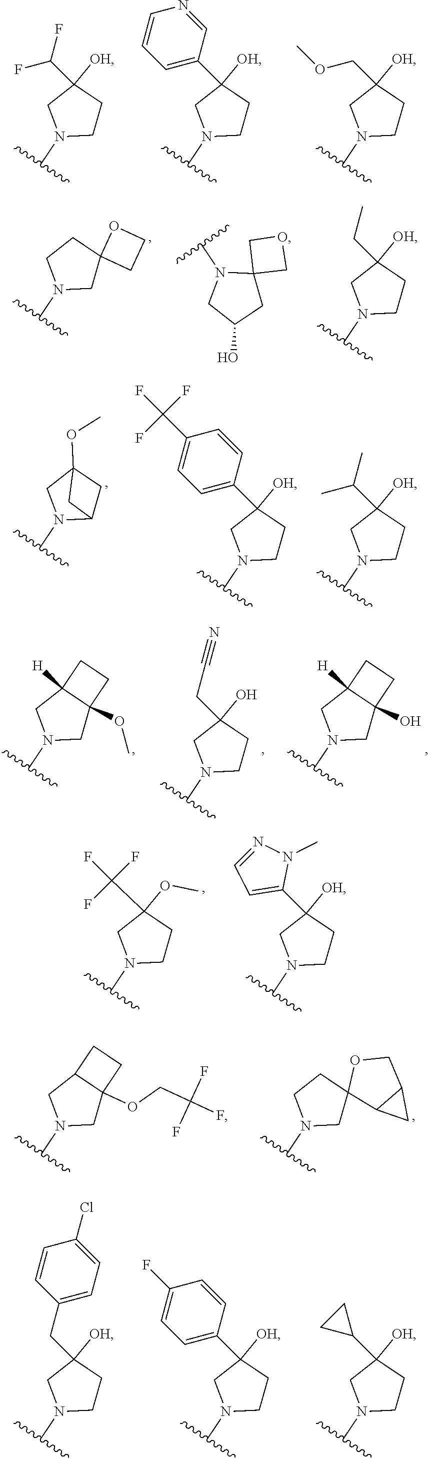

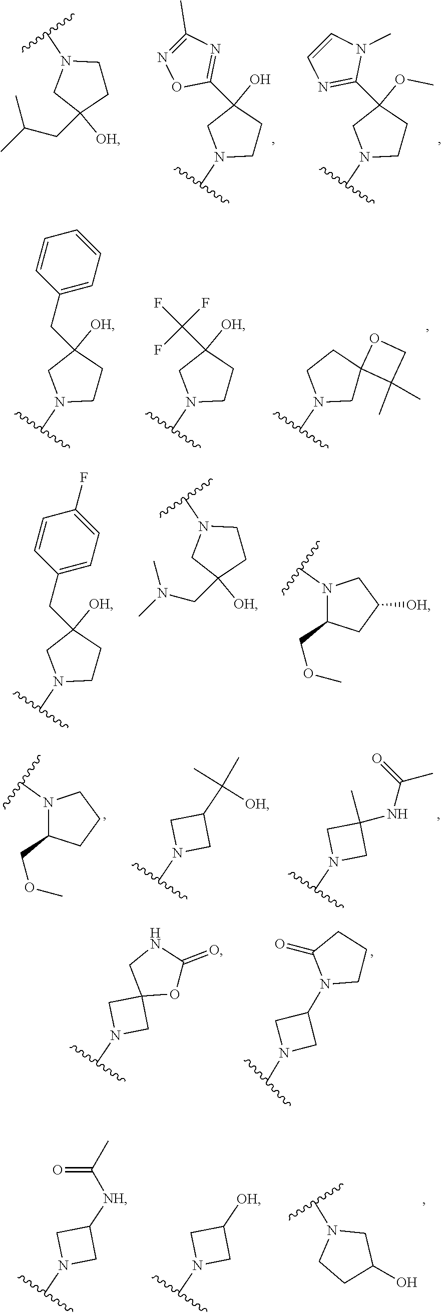

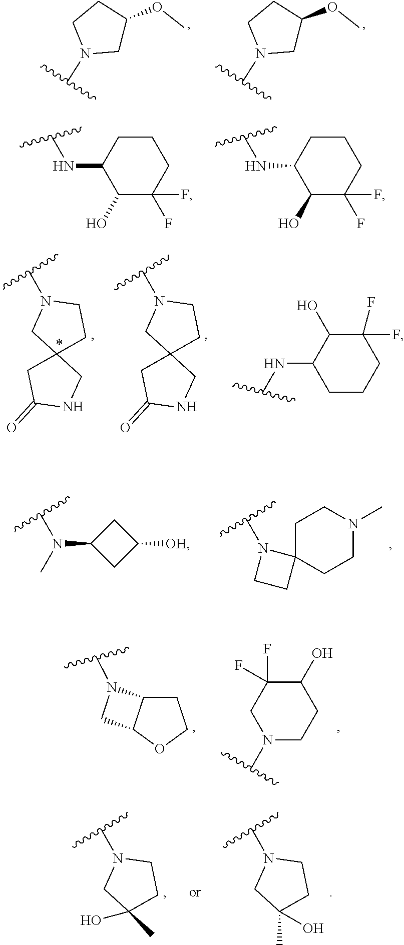

In Embodiment 10 of this disclosure are compounds of Formula I, or Embodiments 1 to 9, or a pharmaceutically acceptable salt of any of the foregoing, wherein R³ is hydrogen, —C₁₋₆ alkyl, —C₃₋₆cycloalkyl, —C₁₋₆haloalkyl, —C₁₋₆alkyl-O—C₁₋₆alkyl, or —C₁₋₆alkyl-OH.

In Embodiment 11 of this disclosure are compounds of Formula I, or Embodiments 1 to 10, or a pharmaceutically acceptable salt of any of the foregoing, wherein R³ is hydrogen or —CH₃.

In Embodiment 12 of this disclosure are compounds of Formula I, or Embodiments 1 to 11, or a pharmaceutically acceptable salt of any of the foregoing, wherein R⁴ is hydrogen, halogen, —C₁₋₆alkyl, —C(O)NH—C₀₋₆alkyl, —C₀₋₆alkyl-(O)OC₁₋₆alkyl, —NH₂, —C₁₋₆haloalkyl, or —C₃₋₆cycloalkyl.

In Embodiment 13 of this disclosure are compounds of Formula I, or Embodiments 1 to 12, or a pharmaceutically acceptable salt of any of the foregoing, wherein R⁴ is hydrogen, methyl, —C(O)NH—CH₃, —C(O)OCH₃, or —CH₂C(O)OCH₂CH₃.

In Embodiment 14 of this disclosure are compounds of Formula I, or Embodiments 1 to 13, or a pharmaceutically acceptable salt of any of the foregoing, wherein R⁵ is hydrogen, halogen, —C₁₋₆alkyl, or C₁₋₆alkyl-(O)OC₁₋₆alkyl.

In Embodiment 15 of this disclosure are compounds of Formula I, or Embodiments 1 to 14, or a pharmaceutically acceptable salt of any of the foregoing, wherein R⁵ is hydrogen.

In Embodiment 16 of this disclosure are compounds of Formula I, or Embodiments 1 to 15, or a pharmaceutically acceptable salt of any of the foregoing, wherein R⁶ is hydrogen, halogen, or —C₁₋₆alkyl.

In Embodiment 17 of this disclosure are compounds of Formula I, or Embodiments 1 to 16, or a pharmaceutically acceptable salt of any of the foregoing, wherein R⁶ is hydrogen, —F, or methyl.

In Embodiment 18 of this disclosure are compounds of Formula I, or Embodiments 1 to 17, or a pharmaceutically acceptable salt of any of the foregoing, wherein X is from —O—, —NH—, —N—C₁₋₆alkyl-, or —S—.

In Embodiment 19 of this disclosure are compounds of Formula I, or Embodiments 1 to 18, or a pharmaceutically acceptable salt of any of the foregoing, wherein X is —O— or —NH—.

In Embodiment 20 of this disclosure are compounds of Formula I, or Embodiments 1 to 19, or a pharmaceutically acceptable salt of any of the foregoing, wherein Y is —O—, —S—, —N—, —C—R¹—, —C(H)—R¹—, —NR¹—, or —CH₂—.

In Embodiment 21 of this disclosure are compounds of Formula I, or Embodiments 1 to 20, or a pharmaceutically acceptable salt of any of the foregoing, wherein Z is —C—R¹, —C(H)—R¹, —NH—, —S—, or —O—.

In Embodiment 22 of this disclosure are compounds of Formulas I or Ia, or a pharmaceutically acceptable salt of any of the foregoing, is the compound of formula Ia

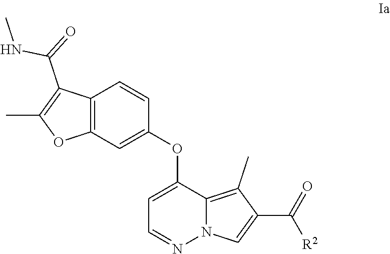

R² is selected from
   (1) —O—C₁₋₆alkyl, and (2)

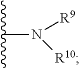

each of R⁹ and R¹⁰ is independently selected from:
   (1) hydrogen,
   (2) —C₀₋₆alkyl-(3-6 membered heterocyclyl) wherein the heterocyclyl has 1-3 ring heteroatoms selected from N, O, and S, and is unsubstituted or substituted with 1-3 substituents independently selected from R^{d},
   (3) —C₁₋₆alkyl, unsubstituted or substituted with 1-3 substituents independently selected from R^{d},
   (4) —C₁₋₆ alkyl-O—C₁₋₆alkyl, unsubstituted or substituted with 1-3 substituents independently selected from R^{d},
   (5) —C₀₋₆alkyl-C₃₋₆cycloalkyl, wherein the cycloalkyl is unsubstituted or substituted with 1-3 substituents independently selected from R^{d}, and
   (6) 5-10 membered bicyclic or tricyclic cycloalkyl, wherein the cycloalkyl is unsubstituted or substituted with 1-3 substituents independently selected from R^{d};
When present, R^{d} is selected from
   (1) —C₁₋₆alkyl,
   (2) halo,
   (3) —NH₂,
   (4) ═O, and
   (5) —OH;
or alternatively, R⁹ and R¹⁰ together with the nitrogen to which they are attached form a 3-10 membered heterocyclyl having 0-3 ring heteroatoms independently selected from N, O, and S, in addition to the nitrogen to which R⁹ and R¹⁰ are attached, wherein the heterocyclyl is optionally substituted with 1-4 substituents independently selected from R^{e}; when present, R^{e} is selected from
   (1) —C₀₋₆alkyl-OH,
   (2) —C₁₋₆alkyl, unsubstituted or substituted with 1-3 substituents selected from —OH,
   (3) —C₀₋₆alkyl-cyano,
   (4) —C₀₋₆alkyl-O—C₁₋₃alkyl,
   (5) —C₀₋₃alkyl-(5-6 membered monocyclic heteroaryl), wherein the heteroaryl has 1-3 ring heteroatoms selected from N, O, and S, and is unsubstituted or substituted with 1-3 substituents selected from C₁₋₆alkyl,
   (6) halo,
   (7) —C₁₋₆haloalkyl,
   (8) C₀₋₆alkyl-aryl, wherein the aryl is unsubstituted or substituted with 1-3 substituents independently selected from C₁₋₆haloalkyl and halo,
   (9) —C₃₋₆cycloalkyl,
   (10) —C₀₋₆alkyl-N, unsubstituted or substituted with 1-3 substituents independently selected from hydrogen and C₁₋₆alkyl,
   (11) —NH—C(O)C₁₋₆alkyl,
   (12) —C₀₋₃alkyl-(3-6 membered monocyclic heterocyclyl), wherein the heterocyclyl has 1-3 ring heteroatoms selected from N, O, and S, and is unsubstituted or substituted with 1 ═O substituent;
   (13) —O—C₁₋₆haloalkyl,
   (14) ═O,
   (15) hydrogen, and
   (16) —C(O)OC₁₋₆alkyl.

In Embodiment 23 of the disclosure are compounds of Formulas I or Ia, or Embodiments 1 to 22, or a pharmaceutically acceptable salt of any of the foregoing, wherein R² is
(1) —OC₁₋₂alkyl,
(2) —NH—C₀₋₃alkyl-3-6 membered heterocyclyl, wherein the heterocyclyl has 1 ring heteroatom selected from S and O, unsubstituted or substituted with 1-3 substituents independently selected from —C₁₋₆alkyl, halo, ═O, and —OH,
(3) —NH—C₁₋₅alkyl, unsubstituted or substituted with 1-3 substituents independently selected from —C₁₋₆alkyl, halo, ═O, and —OH,
(4) —NH—C₀₋₃alkyl-C₃₋₆cycloalkyl, unsubstituted or substituted with 1-3 substituents independently selected from —C₁₋₆alkyl, halo, ═O, and —OH,
(5) 3-7 membered monocyclic heterocyclyl having 0-3 ring heteroatoms independently selected from N, O, and S, in addition to the nitrogen to which R⁹ and R¹⁰ are attached, wherein the monocyclic heterocyclyl is unsubstituted or substituted with 1-4 substituents independently selected from: —C₁₋₆alkyl, unsubstituted or substituted with 1-3 substituents selected from —OH; —NH₂; —C₀₋₆alkyl-cyano; —C₀₋₆alkyl-O—C₁₋₃alkyl; —C₀₋₃alkyl-(5-6 membered monocyclic heteroaryl) having 1-3 ring heteroatoms selected from N, O, and S, unsubstituted or substituted with 1-3 substituents selected from —C₁₋₆alkyl; halo; —OH; —C₁₋₆haloalkyl; C₀₋₆alkyl-aryl, wherein the aryl is unsubstituted or substituted with 1-3 substituents independently selected from —C₁₋₆haloalkyl, and halo; —C₃₋₆cycloalkyl; —C₀₋₆alkyl-N, unsubstituted or substituted with 1-3 substituents independently selected from hydrogen and —C₁₋₆alkyl; —NH—C(O)C₁₋₆alkyl; —N(CH₃)—C(O)C₁₋₆alkyl; and —C₀₋₃alkyl-3-6 membered monocyclic heterocyclyl having 1-3 ring heteroatoms independently selected from N, O, and S, unsubstituted or substituted with 1-3 ═O substituents,
(6) 5-10 membered spiro bicyclic heterocyclyl having 0-3 ring heteroatoms independently selected from N, O, and S, in addition to the nitrogen to which R⁹ and R¹⁰ are attached, wherein the spiro bicyclic heterocyclyl is unsubstituted or substituted with 1-3 substituents independently selected from cyano, ═O, —OH, and —C₁₋₆alkyl,
(7) 5-10 membered bridged heterocyclyl having 0-3 ring heteroatoms independently selected from N, O, and S, in addition to the nitrogen to which R⁹ and R¹⁰ are attached, wherein the bridged heterocyclyl is unsubstituted or substituted with 1-3 substituents independently selected from methyl, —C₀₋₆alkyl-OH, and hydrogen, or
(8) 5-10 membered fused bicyclic heterocyclyl having 0-3 ring heteroatoms independently selected from N, O, and S, in addition to the nitrogen to which R⁹ and R¹⁰ are attached, wherein the fused bicyclic heterocyclyl is unsubstituted or substituted with 1-3 substituents independently selected from halo, hydrogen, —C(O)OC₁₋₆alkyl, —O—C₁₋₆alkyl, —OH, and —O—C₁₋₆haloalkyl.

In Embodiment 24 of the disclosure are compounds of Formulas I or Ia, or Embodiments 1 to 23, or a pharmaceutically acceptable salt of any of the foregoing, wherein R² is
(1) —OCH₂CH₃,
(2) —NH-thietanyl, substituted with 2 ═O substituents and 1 methyl substituent; —NH— tetrahydrothiopyranyl, substituted with 2 ═O substituents and 1 methyl substituent; or —N(CH₃)CH₂-pyrrolidinyl, wherein the pyrrolidinyl is substituted 2 substituents independently selected from methyl and ═O,
(3) —NH—CH₂C(CH₃)₂CH₂OCH₃,
(4) —NH-cyclohexnanyl, substituted with 1-3 substituents independently selected from —OH and —F; —N(CH₃)-cyclohexanyl substituted with 1 —OH substituent; or —NHCH(CH₃)-cyclopropanyl,
(5) pyrrolidinyl, unsubstituted or substituted with 1-3 substituents independently selected from: —OH; pyrazolyl, unsubstituted or substituted with 1 methyl substituent; —CH₂OCH₃; —C₀₋₁alkyl-cyano; —N(CH₃)C(O)CH₃; —CF₃; —C₁₋₄alkyl; —C₀₋₁alkyl-phenyl, unsubstituted or substituted with 1 substituent selected from —C₁, —F, and —CF₃; —OCH₃; cyclopropyl; oxadiazolyl, substituted with 1 methyl substituent; and —CH₂N(CH₃)₂,
(6) azetidinyl, unsubstituted or substituted with 1-3 substituents independently selected from —C₀₋₃alkyl-cyano, —OCH₃, pyrazole, —CH₂-triazole, —OH, —C₁₋₂alkyl, —CH(CH₃)₂, —CH₂OCH₃, —F, —CHF₂, —CF₃, —NH₂, cyclopropyl, and —CH₂N(CH₃)₂,
(7) morpholinyl, substituted with 1 substituent selected from —CH(CH₃)₂ and methyl,
(8) azepanyl, unsubstituted or substituted with 1 —OH substituent,
(9) oxazepanyl,
(10) oxa-azaspiro[3.4]octanyl, unsubstituted or substituted with 2 methyl substituents; diazaspiro[3.4]octanyl, substituted with 1 ═O substituent and 1 hydrogen substituent; azaspiro[3.3]heptanyl, substituted with 1 substituent selected from —OH and cyano; diazaspiro[4.4]nonanyl, substituted with 1 ═O substituent; oxa-azaspiro[4.4]nonanyl; oxa-azaspiro[3.5]nonanyl; oxa-azaspiro[4.5]decanyl, unsubstituted or substituted with 1 ═O substituent; azaspiro[2.3]hexanyl; or oxa-azaspiro[bicyclo[3.1.0]hexane-pyrrolidinyl],
(11) azabicyclo[2.1.1]hexanyl, unsubstituted or substituted with 1-3 substituents selected from methyl, —CH₂OH, —OH, —C(CH₃)₂OH, —OCH₃; azabicyclo[3.1.0]hexanyl, unsubstituted or substituted with 1 —C(O)OCH₂CH₃ substituent; oxa-azabicyclo[2.2.1]heptanyl; azabicyclo[3.1.1]heptanyl; azabicyclo[2.2.1]heptanyl, unsubstituted or substituted with 1-4 substituents independently selected from —OH, hydrogen, and methyl; or azabicyclo[3.2.0]heptanyl, substituted with 1-2 substituents independently selected from hydrogen, —OCH₂CF₃, —OCH₃, and —OH, or
(12) hexahydrocyclopenta[c]pyrrolyl, substituted with 2 —F substituents; hexahydrofuro[3,2-b]pyrrolyl; octahydro-indolyl; hexahydropyrano[3,2-c]pyridinyl; hexahydrofuro[3,2-b]pyridinyl; or octahydrobenzo[b][1,4]oxazinyl, substituted with 1 methyl substituent.

In Embodiment 25 of the disclosure are compounds of Formulas I or Ia, or Embodiments 1 to 24, or a pharmaceutically acceptable salt of any of the foregoing wherein R² is

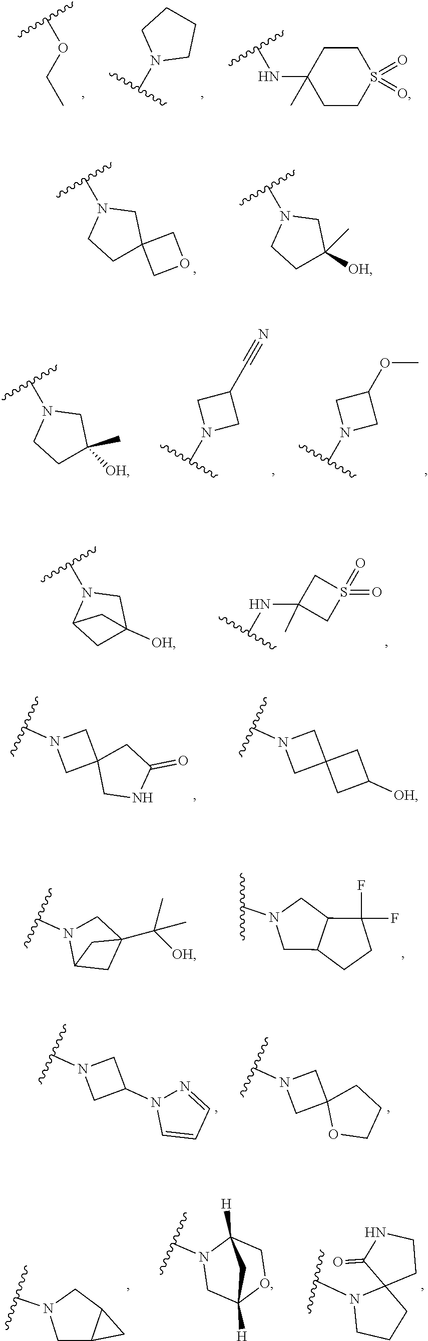

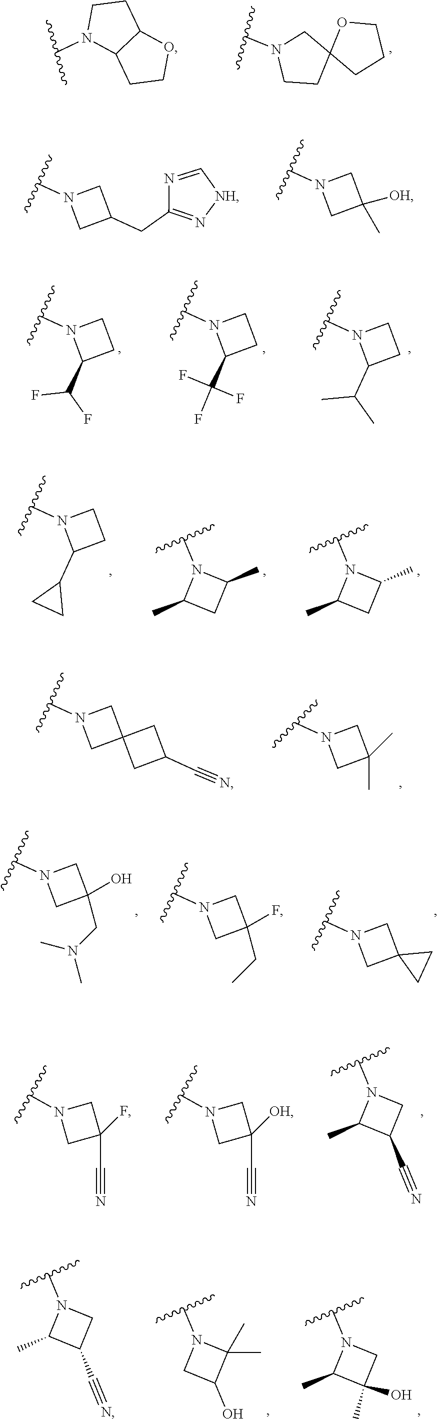

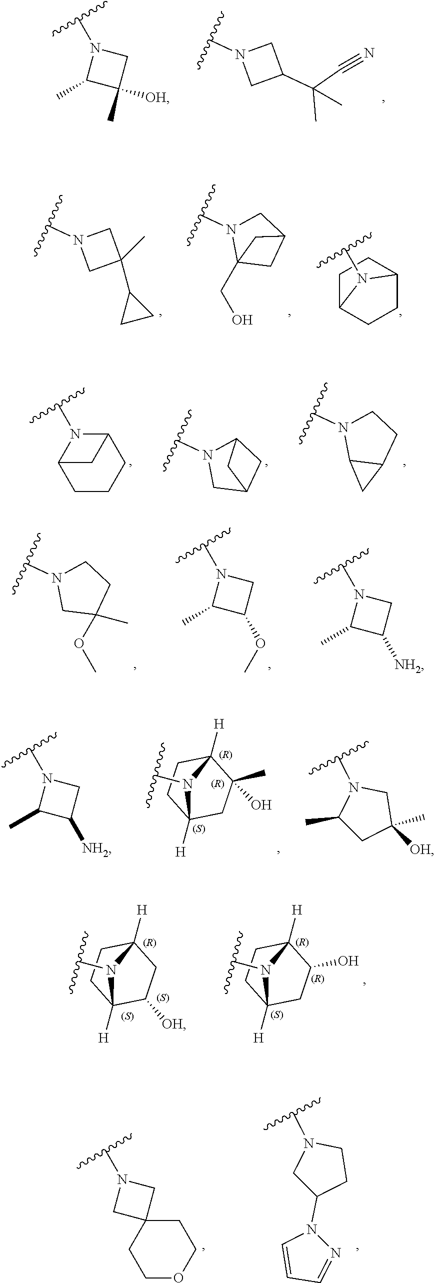

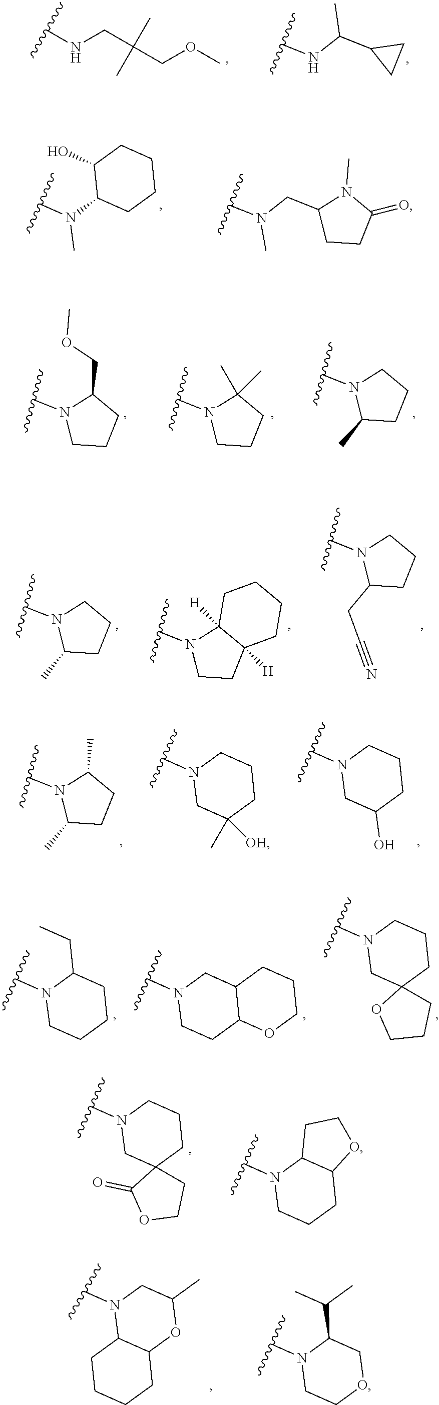

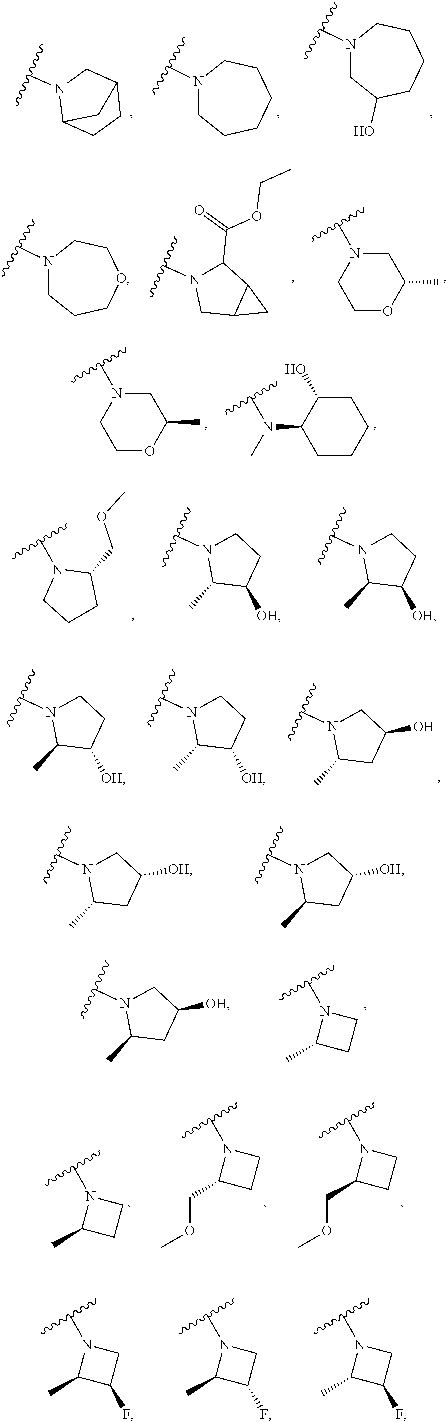

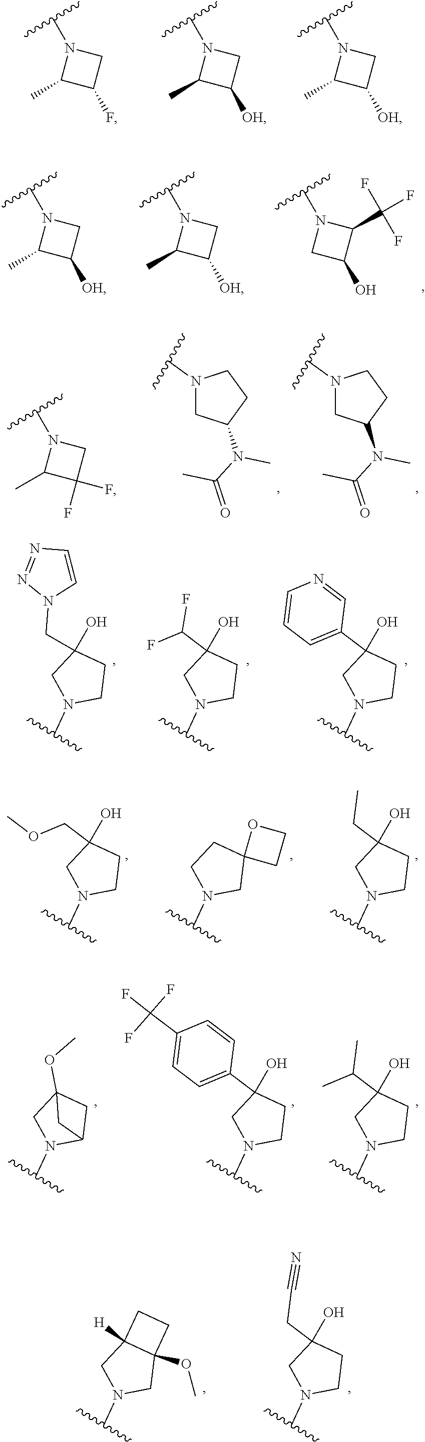

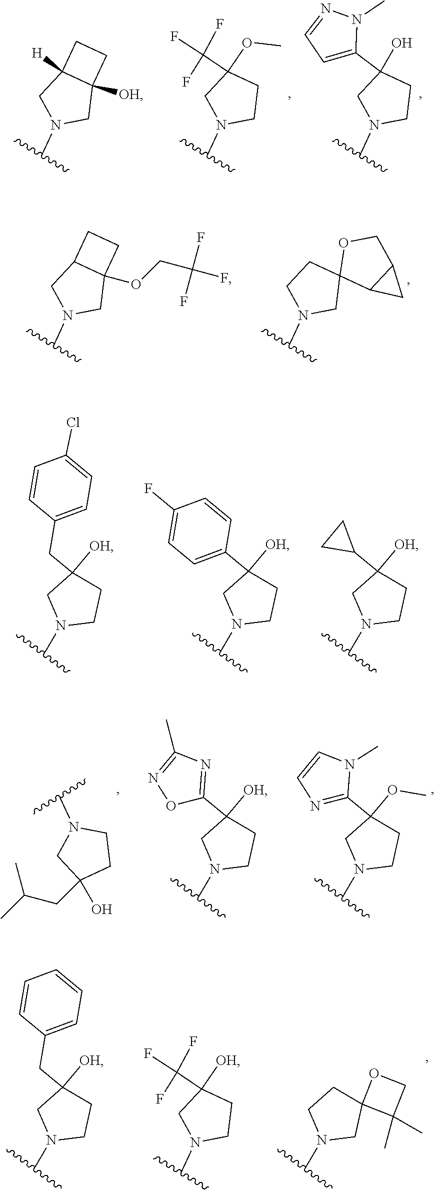

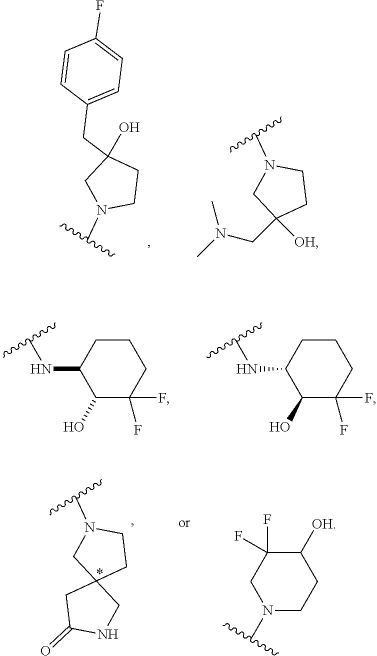

In Embodiment 26 of the disclosure, the compound of formulas I or Ia, or Embodiments 1 to 25, or a pharmaceutically acceptable salt thereof, is:

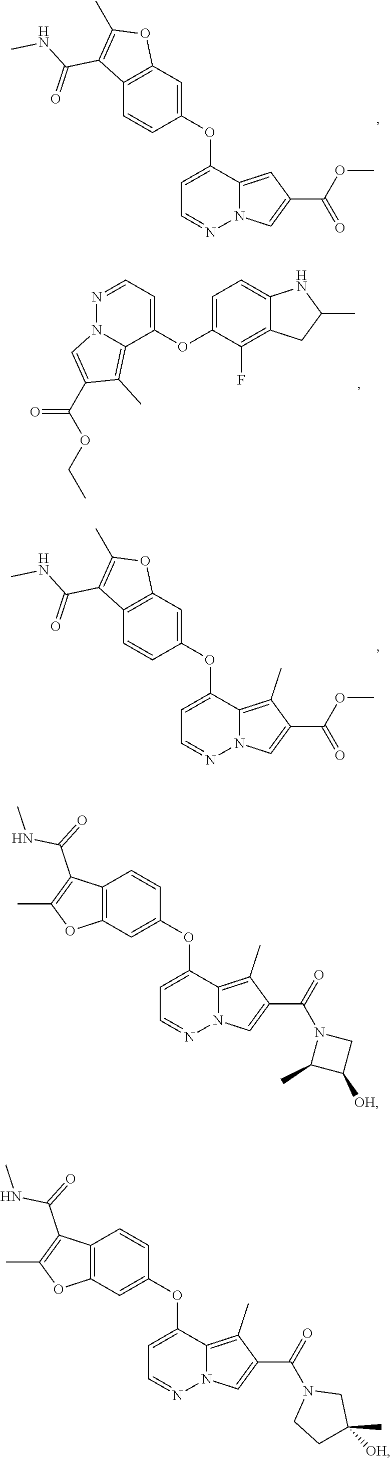

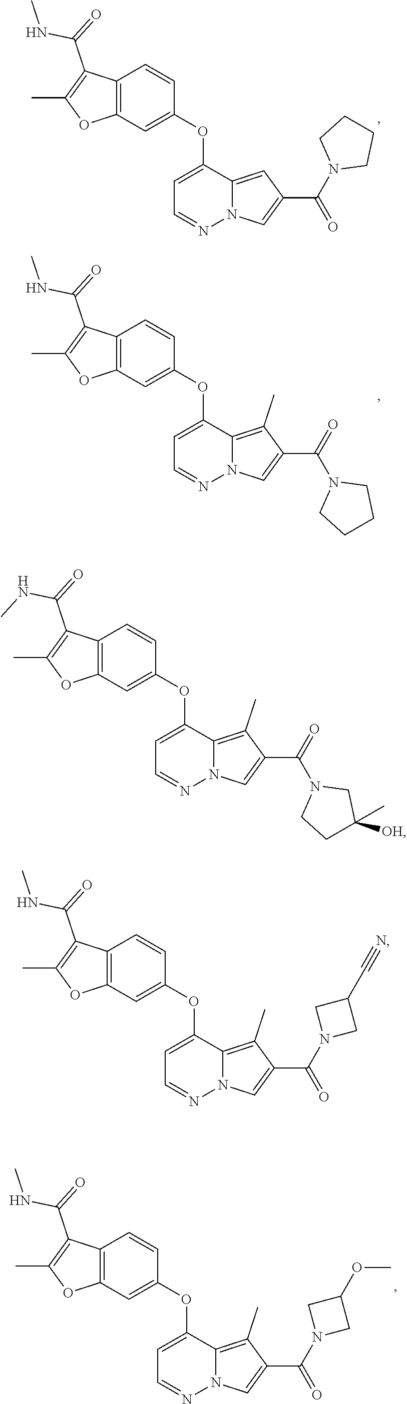

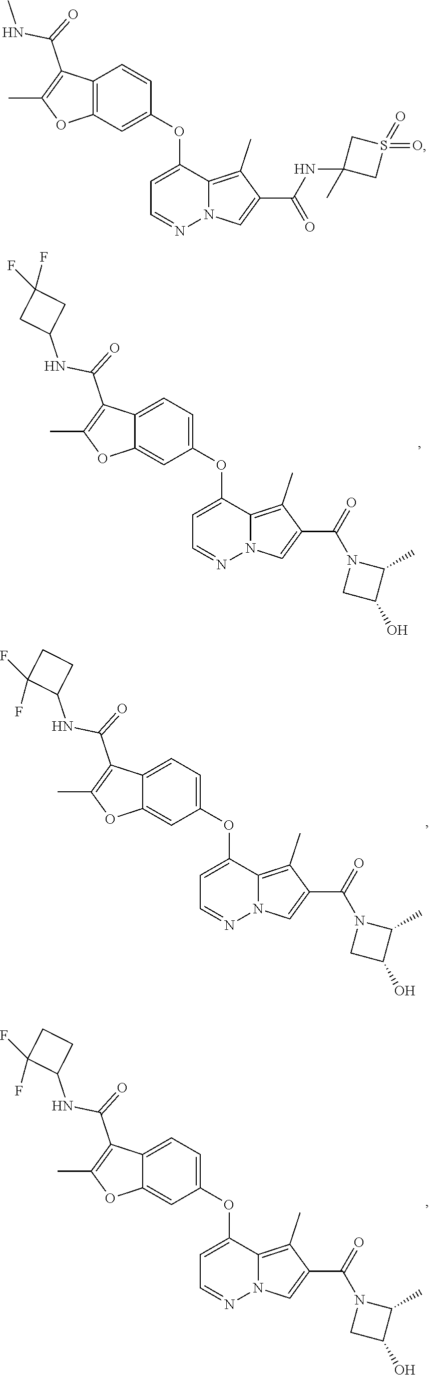 [SMILES: CNC(=O)c1c(C)oc2cc(Oc3ccnn4cc(C(=O)NC5(C)CS(=O)(=O)C5)c(C)c34)ccc12 ; Cc1oc2cc(Oc3ccnn4cc(C(=O)N5C[C@@H](O)[C@H]5C)c(C)c34)ccc2c1C(=O)NC1CC(F)(F)C1 ; Cc1oc2cc(Oc3ccnn4cc(C(=O)N5C[C@@H](O)[C@H]5C)c(C)c34)ccc2c1C(=O)NC1CCC1(F)F ; Cc1oc2cc(Oc3ccnn4cc(C(=O)N5C[C@@H](O)[C@H]5C)c(C)c34)ccc2c1C(=O)NC1CCC1(F)F]

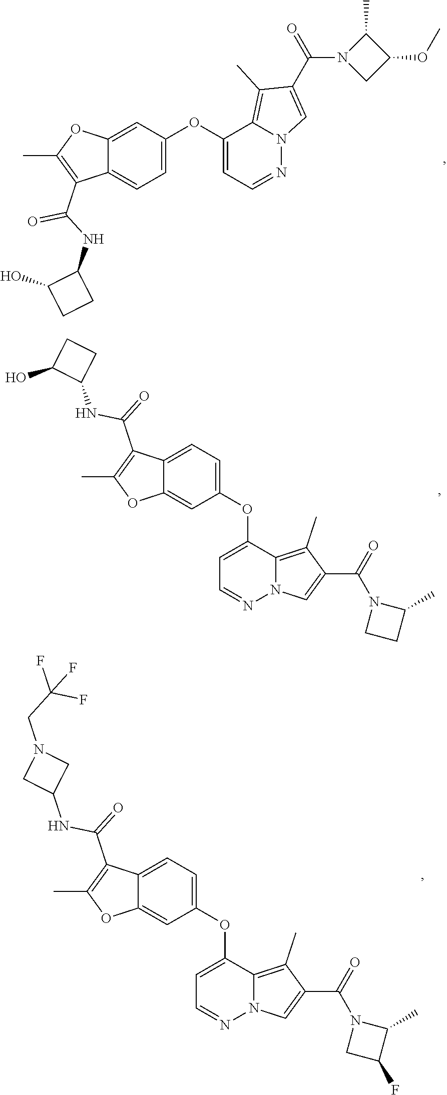

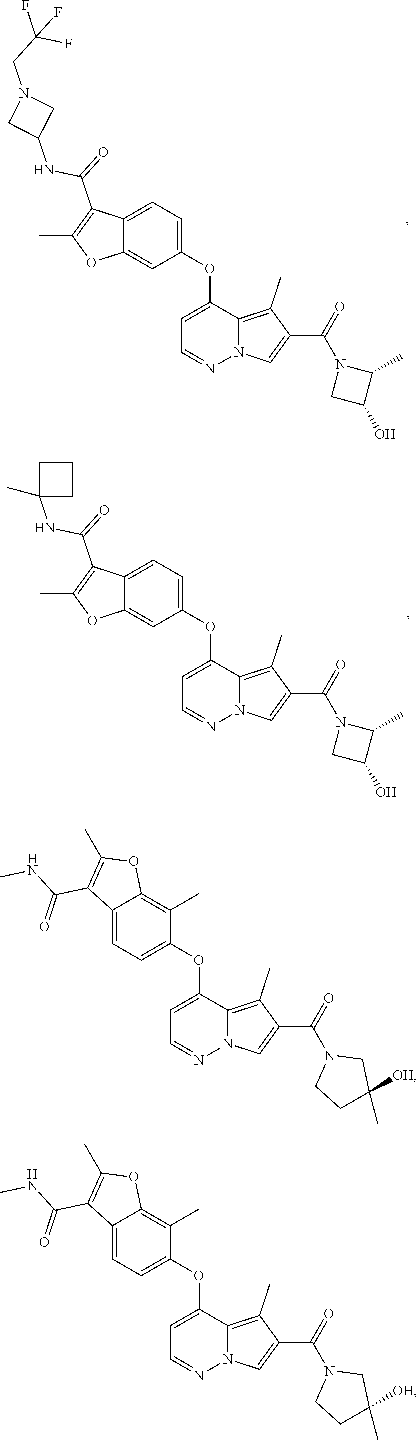

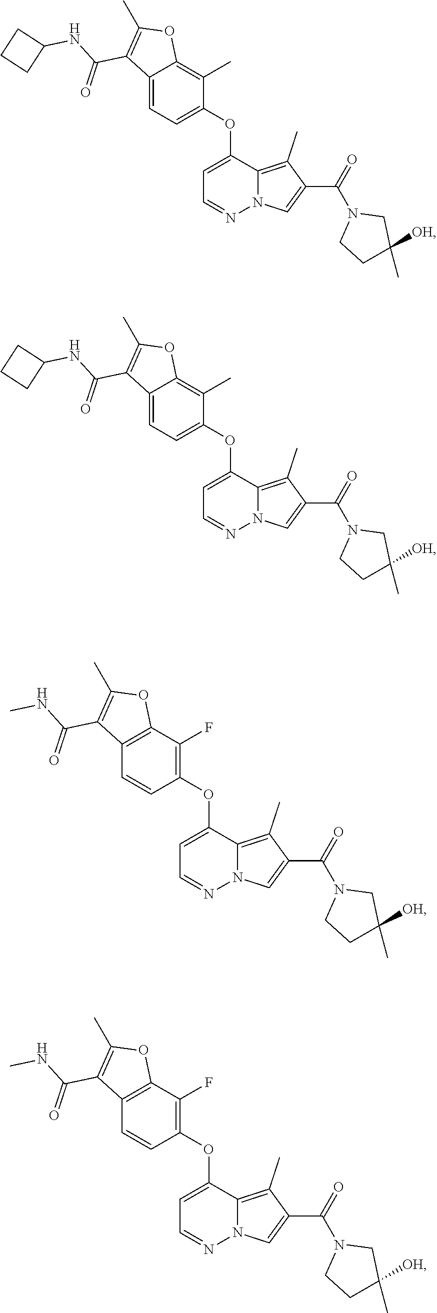 [SMILES: Cc1oc2c(C)c(Oc3ccnn4cc(C(=O)N5CC[C@](C)(O)C5)c(C)c34)ccc2c1C(=O)NC1CCC1 ; Cc1oc2c(C)c(Oc3ccnn4cc(C(=O)N5CC[C@@](C)(O)C5)c(C)c34)ccc2c1C(=O)NC1CCC1 ; CNC(=O)c1c(C)oc2c(F)c(Oc3ccnn4cc(C(=O)N5CC[C@](C)(O)C5)c(C)c34)ccc12 ; CNC(=O)c1c(C)oc2c(F)c(Oc3ccnn4cc(C(=O)N5CC[C@@](C)(O)C5)c(C)c34)ccc12]

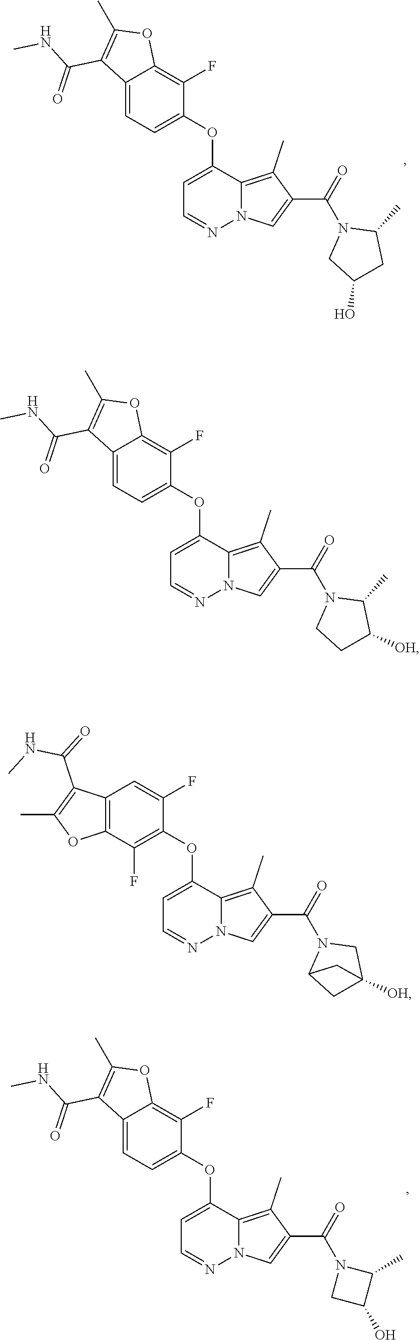

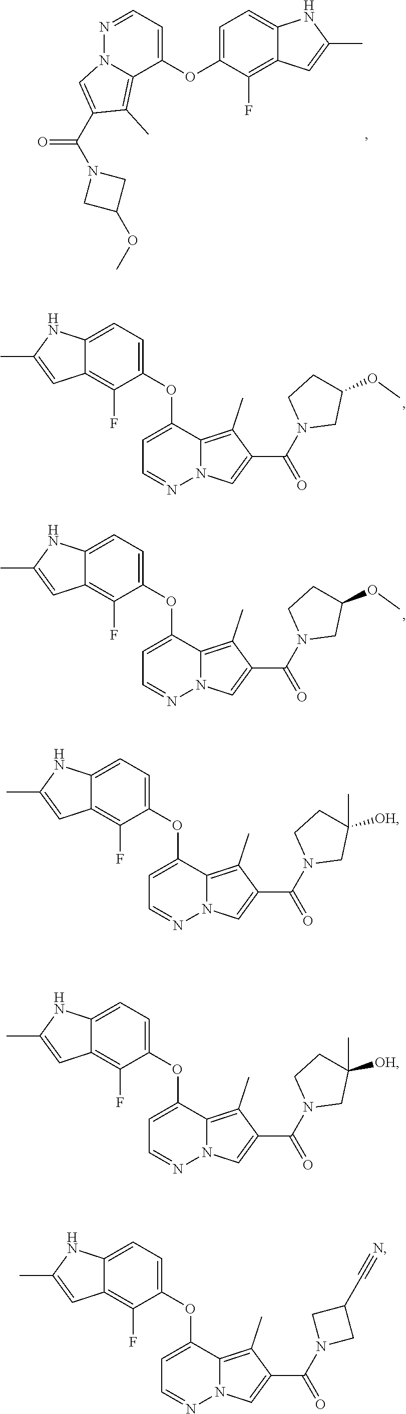

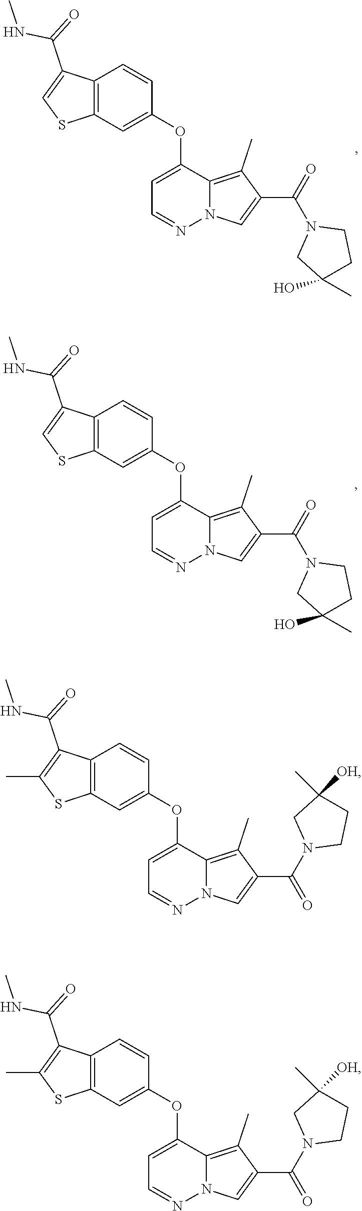

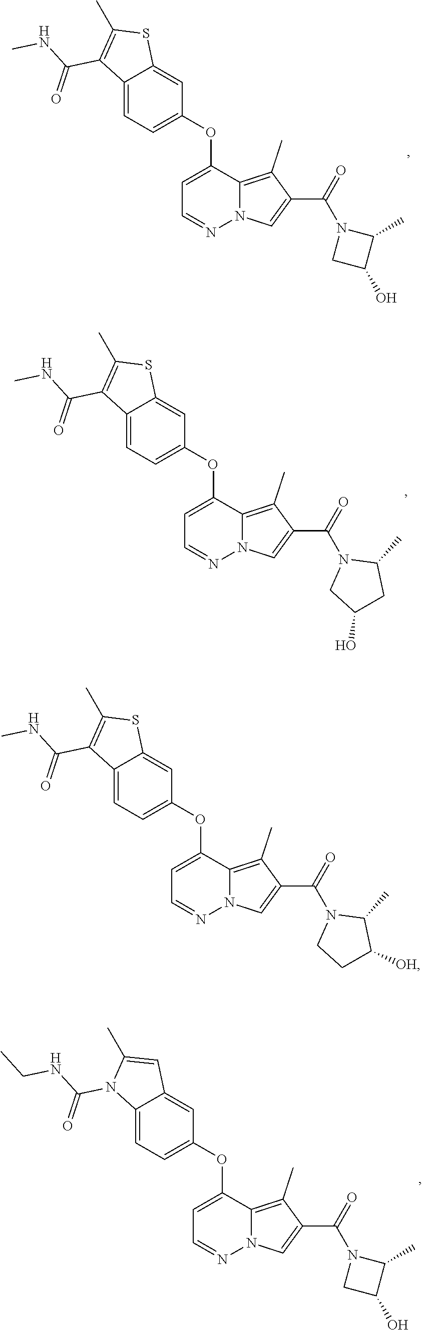

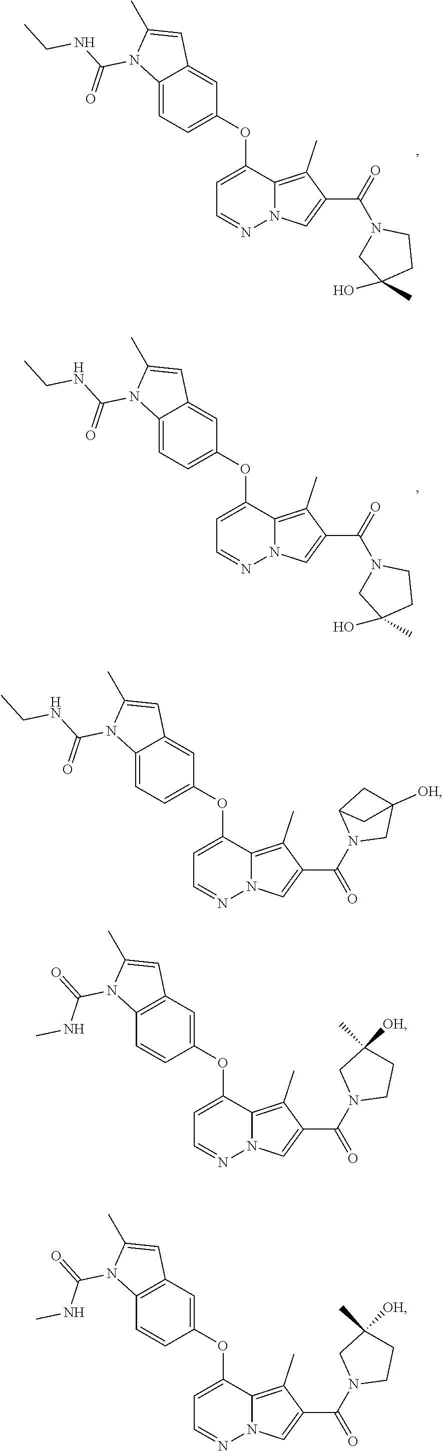 [SMILES: CCNC(=O)n1c(C)cc2cc(Oc3ccnn4cc(C(=O)N5CC[C@](C)(O)C5)c(C)c34)ccc21 ; CCNC(=O)n1c(C)cc2cc(Oc3ccnn4cc(C(=O)N5CC[C@@](C)(O)C5)c(C)c34)ccc21 ; CCNC(=O)n1c(C)cc2cc(Oc3ccnn4cc(C(=O)N5CC6(O)CC5C6)c(C)c34)ccc21 ; CNC(=O)n1c(C)cc2cc(Oc3ccnn4cc(C(=O)N5CC[C@](C)(O)C5)c(C)c34)ccc21 ; CNC(=O)n1c(C)cc2cc(Oc3ccnn4cc(C(=O)N5CC[C@@](C)(O)C5)c(C)c34)ccc21]

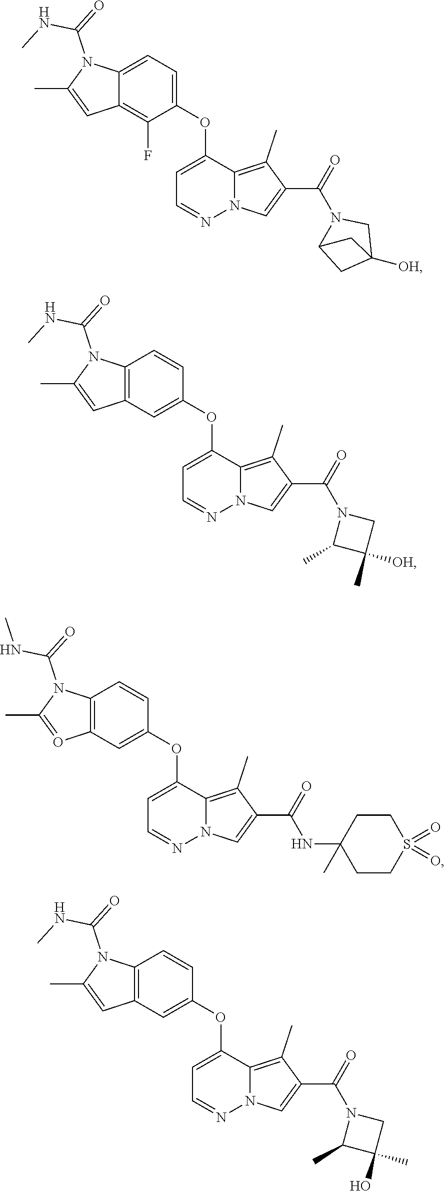

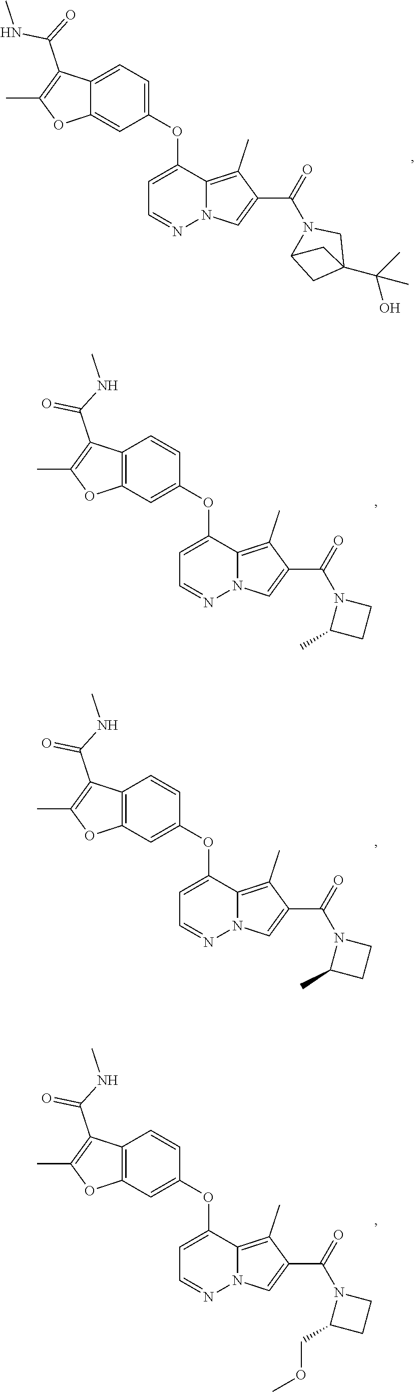

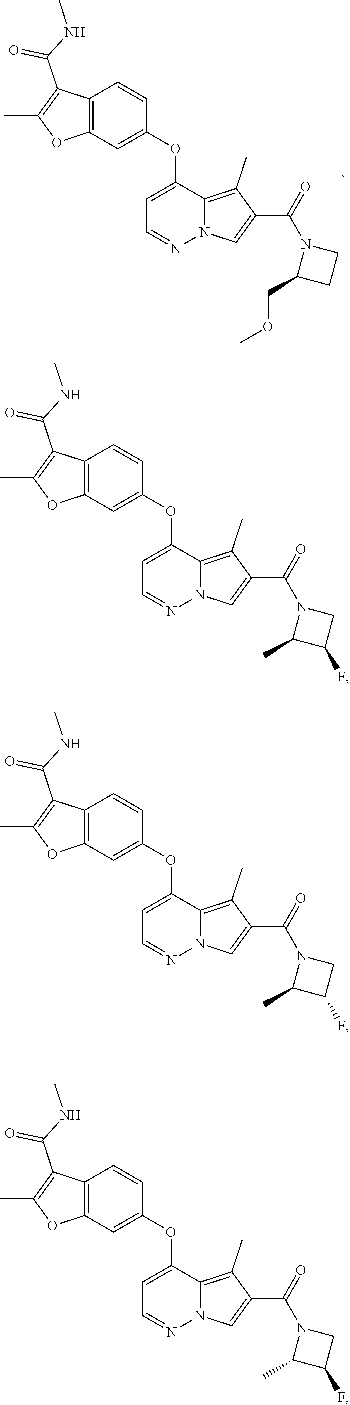 [SMILES: CNC(=O)c1c(C)oc2cc(Oc3ccnn4cc(C(=O)N5CC[C@H]5COC)c(C)c34)ccc12 ; CNC(=O)c1c(C)oc2cc(Oc3ccnn4cc(C(=O)N5C[C@@H](F)[C@H]5C)c(C)c34)ccc12 ; CNC(=O)c1c(C)oc2cc(Oc3ccnn4cc(C(=O)N5C[C@H](F)[C@H]5C)c(C)c34)ccc12 ; CNC(=O)c1c(C)oc2cc(Oc3ccnn4cc(C(=O)N5C[C@@H](F)[C@@H]5C)c(C)c34)ccc12]

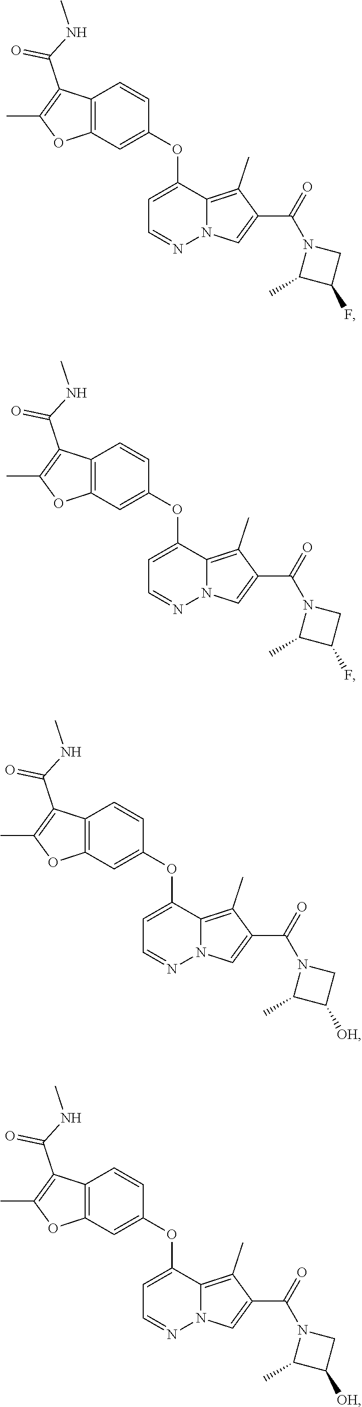 [SMILES: CNC(=O)c1c(C)oc2cc(Oc3ccnn4cc(C(=O)N5C[C@@H](F)[C@@H]5C)c(C)c34)ccc12 ; CNC(=O)c1c(C)oc2cc(Oc3ccnn4cc(C(=O)N5C[C@H](F)[C@@H]5C)c(C)c34)ccc12 ; CNC(=O)c1c(C)oc2cc(Oc3ccnn4cc(C(=O)N5C[C@H](O)[C@@H]5C)c(C)c34)ccc12 ; CNC(=O)c1c(C)oc2cc(Oc3ccnn4cc(C(=O)N5C[C@@H](O)[C@@H]5C)c(C)c34)ccc12]

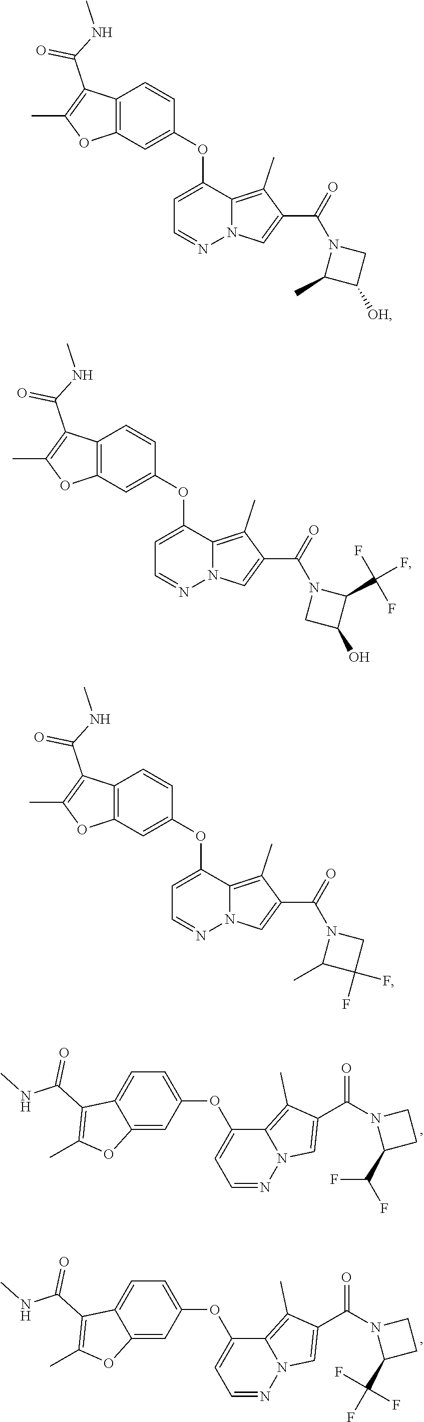 [SMILES: CNC(=O)c1c(C)oc2cc(Oc3ccnn4cc(C(=O)N5C[C@H](O)[C@H]5C)c(C)c34)ccc12 ; CNC(=O)c1c(C)oc2cc(Oc3ccnn4cc(C(=O)N5C[C@H](O)[C@@H]5C(F)(F)F)c(C)c34)ccc12 ; CNC(=O)c1c(C)oc2cc(Oc3ccnn4cc(C(=O)N5CC(F)(F)C5C)c(C)c34)ccc12 ; CNC(=O)c1c(C)oc2cc(Oc3ccnn4cc(C(=O)N5CC[C@H]5C(F)F)c(C)c34)ccc12 ; CNC(=O)c1c(C)oc2cc(Oc3ccnn4cc(C(=O)N5CC[C@H]5C(F)(F)F)c(C)c34)ccc12]

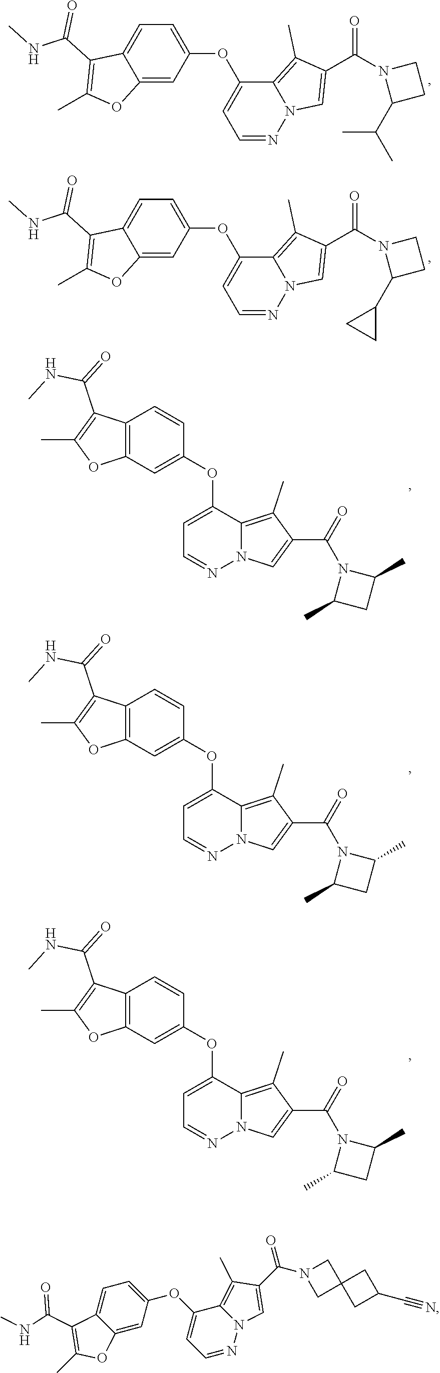

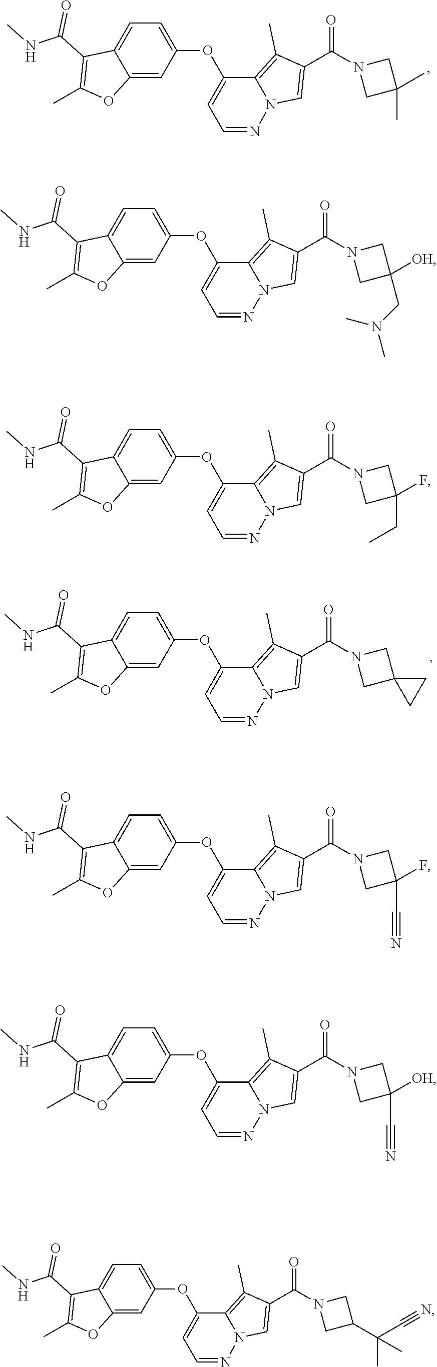

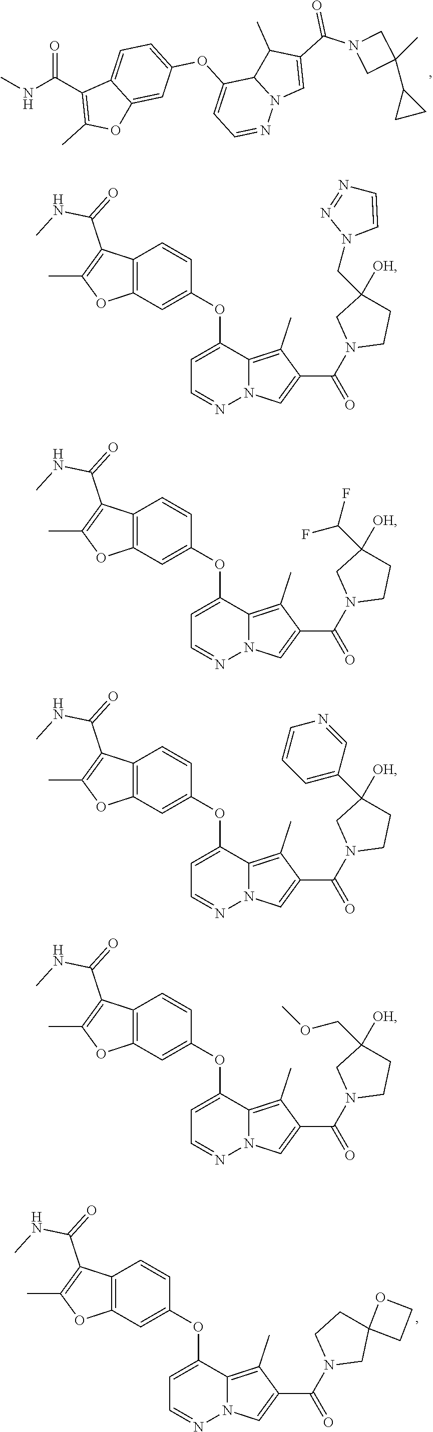 [SMILES: CNC(=O)c1c(C)oc2cc(OC3=CC=NN4C=C(C(=O)N5CC(C)(C6CC6)C5)C(C)C34)ccc12 ; CNC(=O)c1c(C)oc2cc(Oc3ccnn4cc(C(=O)N5CCC(O)(Cn6ccnn6)C5)c(C)c34)ccc12 ; CNC(=O)c1c(C)oc2cc(Oc3ccnn4cc(C(=O)N5CCC(O)(C(F)F)C5)c(C)c34)ccc12 ; CNC(=O)c1c(C)oc2cc(Oc3ccnn4cc(C(=O)N5CCC(O)(c6cccnc6)C5)c(C)c34)ccc12 ; CNC(=O)c1c(C)oc2cc(Oc3ccnn4cc(C(=O)N5CCC(O)(COC)C5)c(C)c34)ccc12 ; CNC(=O)c1c(C)oc2cc(Oc3ccnn4cc(C(=O)N5CCC6(CCO6)C5)c(C)c34)ccc12]

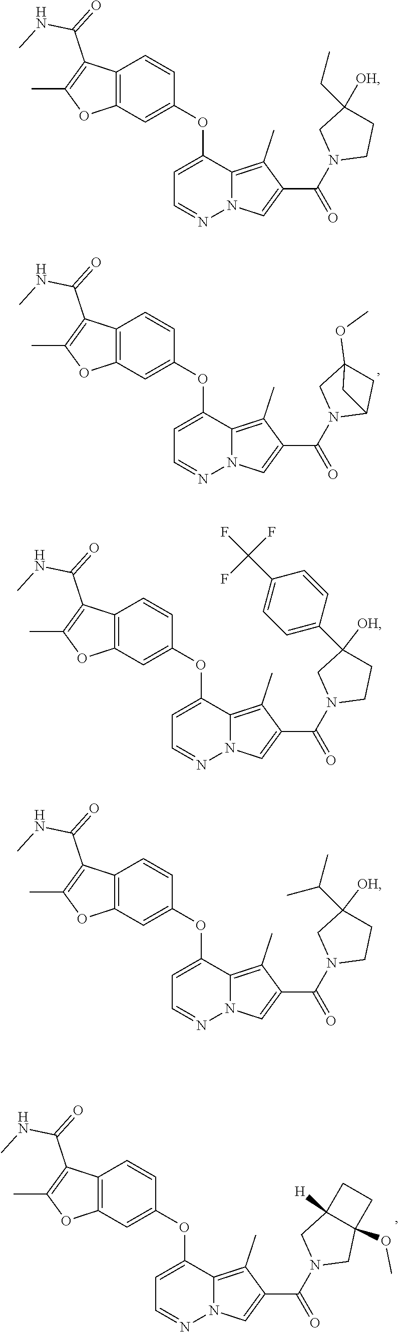

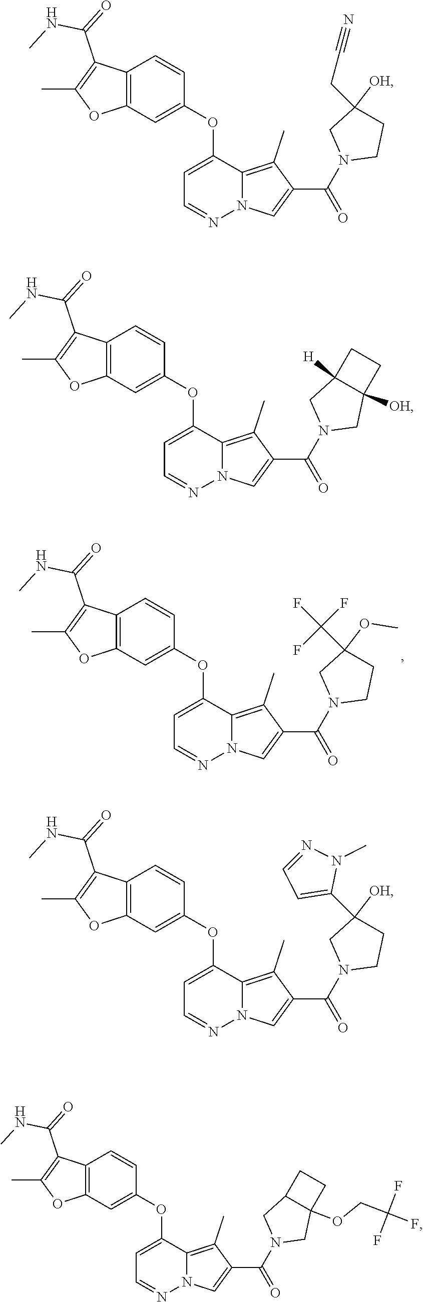

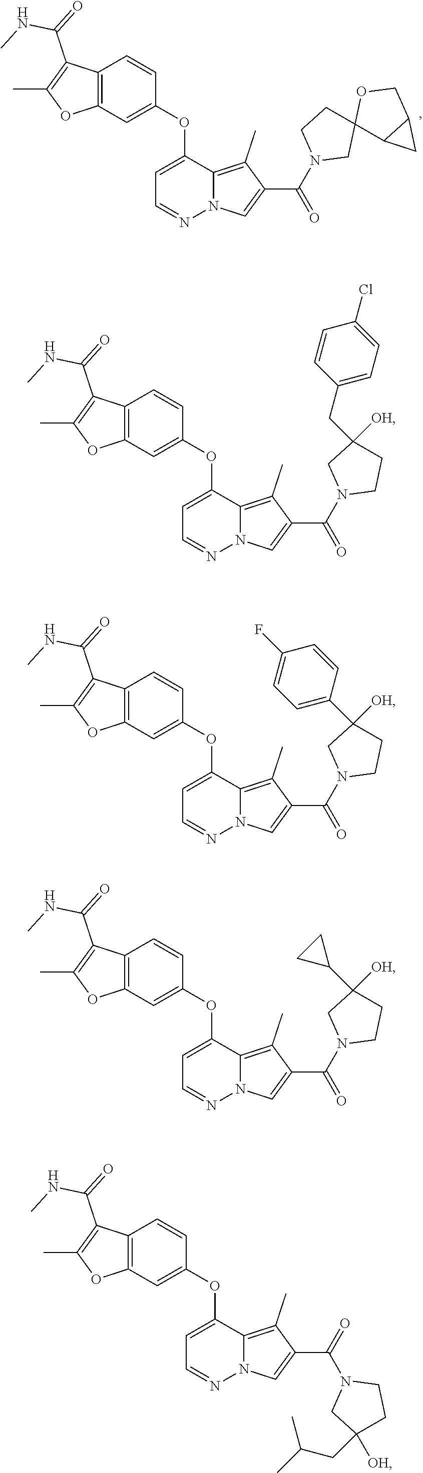 [SMILES: CNC(=O)c1c(C)oc2cc(Oc3ccnn4cc(C(=O)N5CCC6(C5)OCC5CC56)c(C)c34)ccc12 ; CNC(=O)c1c(C)oc2cc(Oc3ccnn4cc(C(=O)N5CCC(O)(Cc6ccc(Cl)cc6)C5)c(C)c34)ccc12 ; CNC(=O)c1c(C)oc2cc(Oc3ccnn4cc(C(=O)N5CCC(O)(c6ccc(F)cc6)C5)c(C)c34)ccc12 ; CNC(=O)c1c(C)oc2cc(Oc3ccnn4cc(C(=O)N5CCC(O)(C6CC6)C5)c(C)c34)ccc12 ; CNC(=O)c1c(C)oc2cc(Oc3ccnn4cc(C(=O)N5CCC(O)(CC(C)C)C5)c(C)c34)ccc12]

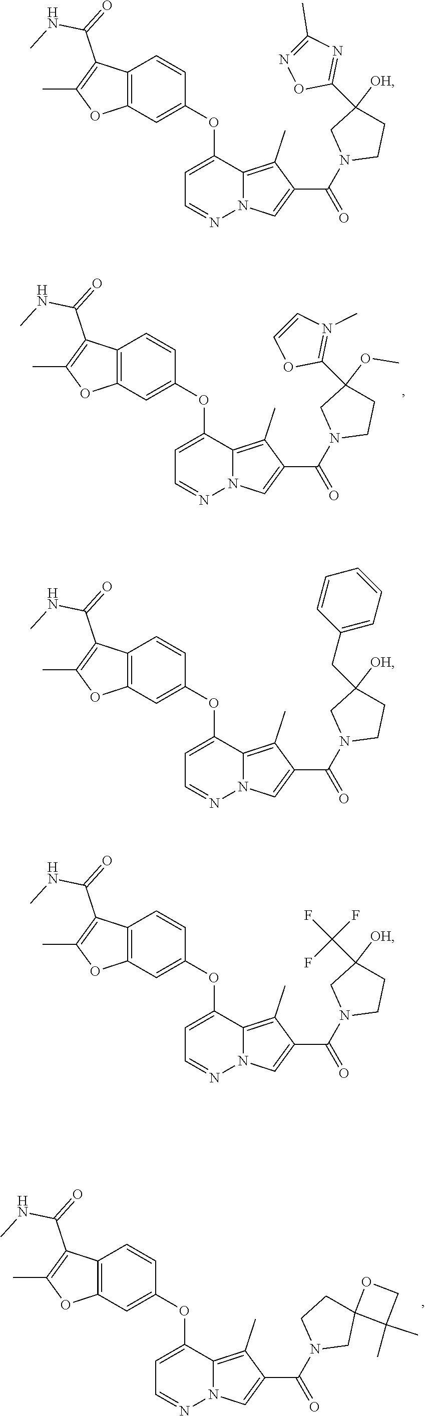

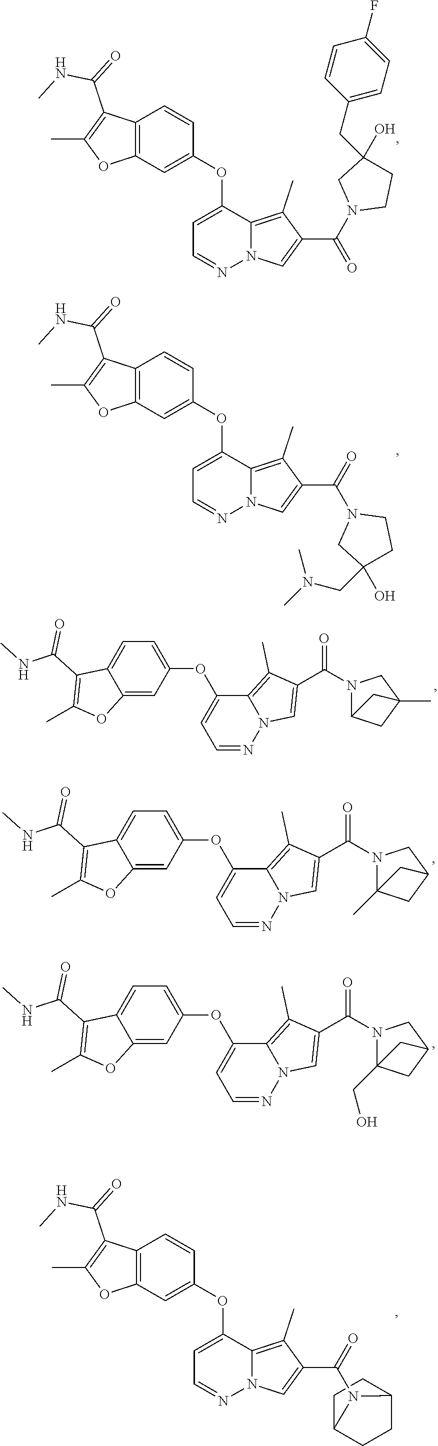

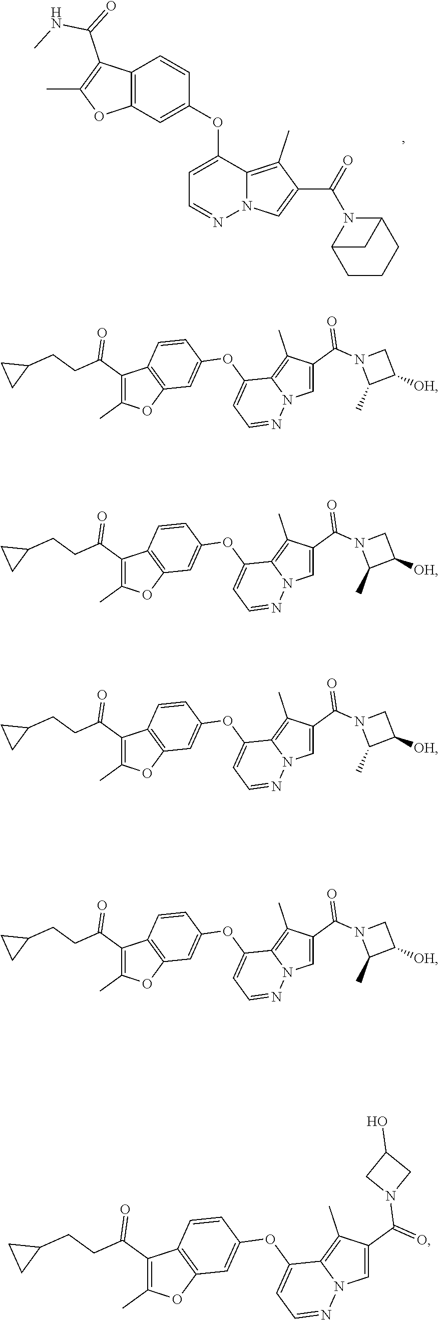 [SMILES: CNC(=O)c1c(C)oc2cc(Oc3ccnn4cc(C(=O)N5C6CCCC5C6)c(C)c34)ccc12 ; Cc1oc2cc(Oc3ccnn4cc(C(=O)N5C[C@H](O)[C@@H]5C)c(C)c34)ccc2c1C(=O)CCC1CC1 ; Cc1oc2cc(Oc3ccnn4cc(C(=O)N5C[C@@H](O)[C@H]5C)c(C)c34)ccc2c1C(=O)CCC1CC1 ; Cc1oc2cc(Oc3ccnn4cc(C(=O)N5C[C@@H](O)[C@@H]5C)c(C)c34)ccc2c1C(=O)CCC1CC1 ; Cc1oc2cc(Oc3ccnn4cc(C(=O)N5C[C@H](O)[C@H]5C)c(C)c34)ccc2c1C(=O)CCC1CC1 ; Cc1oc2cc(Oc3ccnn4cc(C(=O)N5CC(O)C5)c(C)c34)ccc2c1C(=O)CCC1CC1]

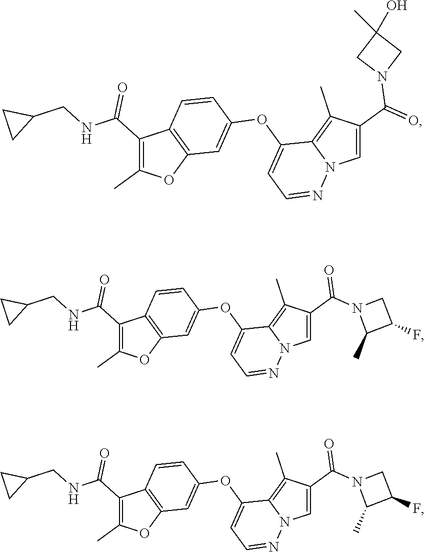 [SMILES: Cc1oc2cc(Oc3ccnn4cc(C(=O)N5CC(C)(O)C5)c(C)c34)ccc2c1C(=O)NCC1CC1 ; Cc1oc2cc(Oc3ccnn4cc(C(=O)N5C[C@H](F)[C@H]5C)c(C)c34)ccc2c1C(=O)NCC1CC1 ; Cc1oc2cc(Oc3ccnn4cc(C(=O)N5C[C@@H](F)[C@@H]5C)c(C)c34)ccc2c1C(=O)NCC1CC1]

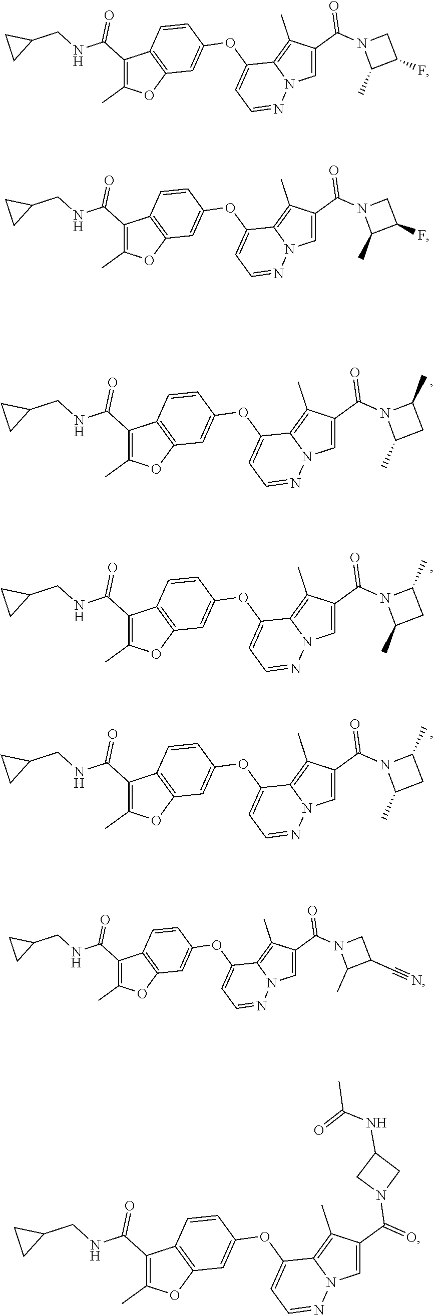

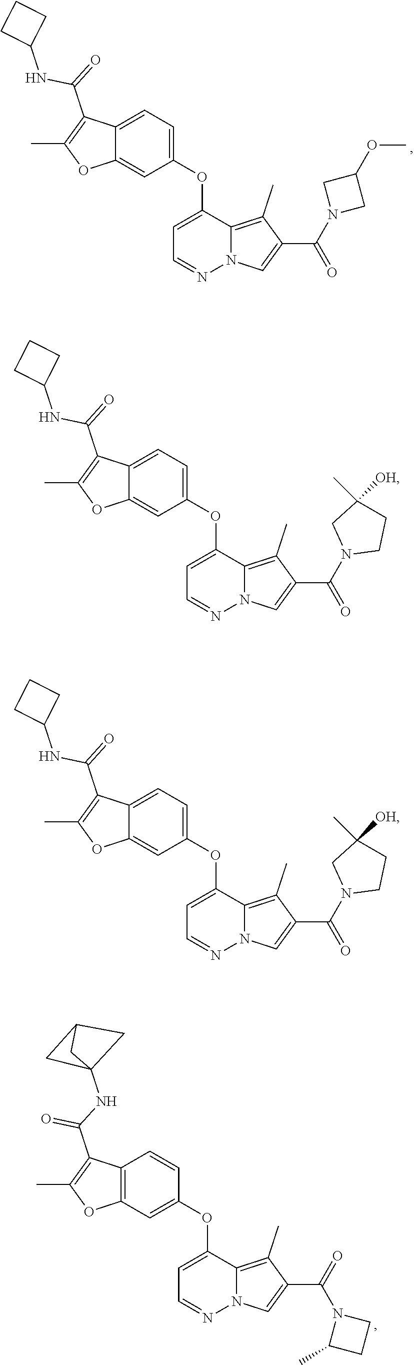

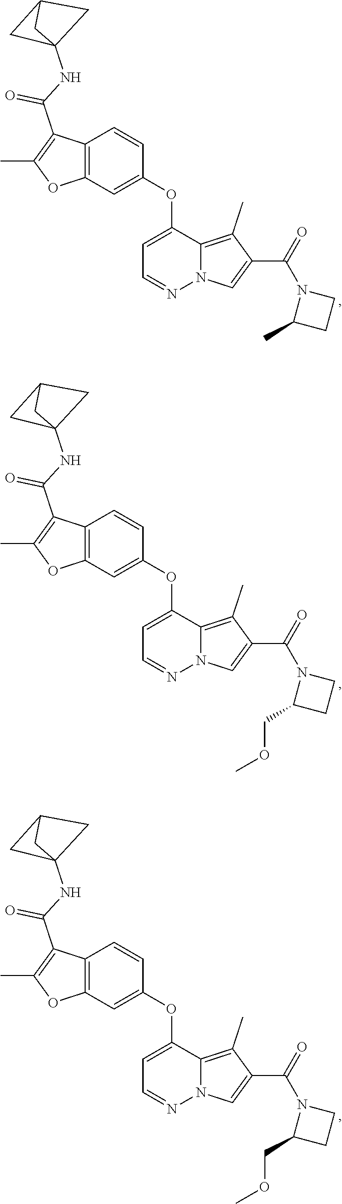 [SMILES: Cc1oc2cc(Oc3ccnn4cc(C(=O)N5CC[C@H]5C)c(C)c34)ccc2c1C(=O)NC12CC(C1)C2 ; COC[C@H]1CCN1C(=O)c1cn2nccc(Oc3ccc4c(C(=O)NC56CC(C5)C6)c(C)oc4c3)c2c1C ; COC[C@@H]1CCN1C(=O)c1cn2nccc(Oc3ccc4c(C(=O)NC56CC(C5)C6)c(C)oc4c3)c2c1C]

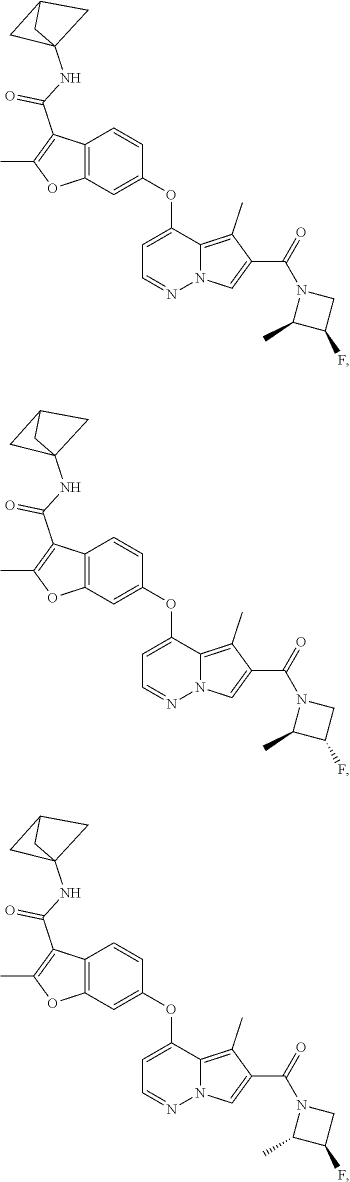 [SMILES: Cc1oc2cc(Oc3ccnn4cc(C(=O)N5C[C@@H](F)[C@H]5C)c(C)c34)ccc2c1C(=O)NC12CC(C1)C2 ; Cc1oc2cc(Oc3ccnn4cc(C(=O)N5C[C@H](F)[C@H]5C)c(C)c34)ccc2c1C(=O)NC12CC(C1)C2 ; Cc1oc2cc(Oc3ccnn4cc(C(=O)N5C[C@@H](F)[C@@H]5C)c(C)c34)ccc2c1C(=O)NC12CC(C1)C2]

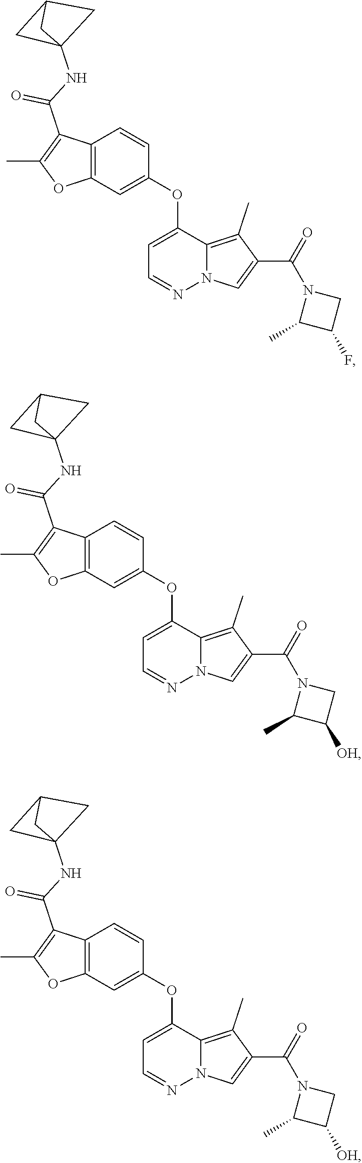 [SMILES: Cc1oc2cc(Oc3ccnn4cc(C(=O)N5C[C@H](F)[C@@H]5C)c(C)c34)ccc2c1C(=O)NC12CC(C1)C2 ; Cc1oc2cc(Oc3ccnn4cc(C(=O)N5C[C@@H](O)[C@H]5C)c(C)c34)ccc2c1C(=O)NC12CC(C1)C2 ; Cc1oc2cc(Oc3ccnn4cc(C(=O)N5C[C@H](O)[C@@H]5C)c(C)c34)ccc2c1C(=O)NC12CC(C1)C2]

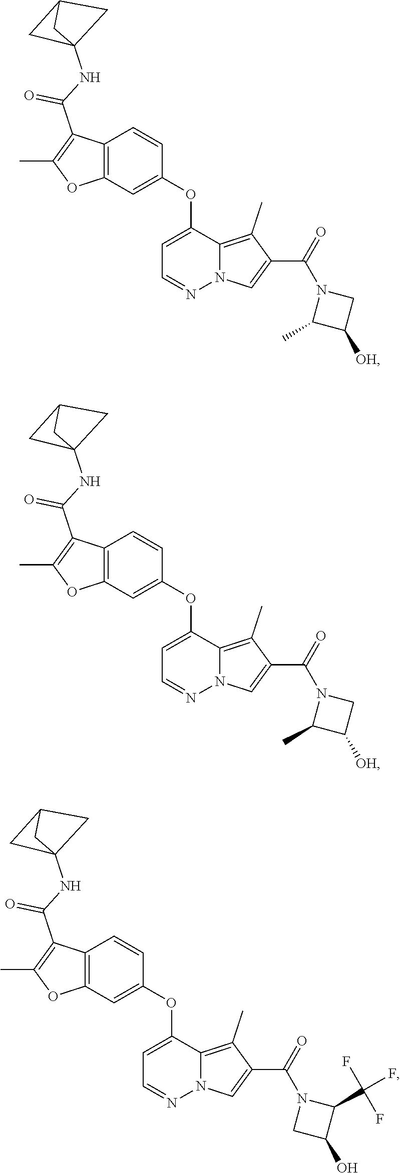 [SMILES: Cc1oc2cc(Oc3ccnn4cc(C(=O)N5C[C@@H](O)[C@@H]5C)c(C)c34)ccc2c1C(=O)NC12CC(C1)C2 ; Cc1oc2cc(Oc3ccnn4cc(C(=O)N5C[C@H](O)[C@H]5C)c(C)c34)ccc2c1C(=O)NC12CC(C1)C2 ; Cc1oc2cc(Oc3ccnn4cc(C(=O)N5C[C@H](O)[C@@H]5C(F)(F)F)c(C)c34)ccc2c1C(=O)NC12CC(C1)C2]

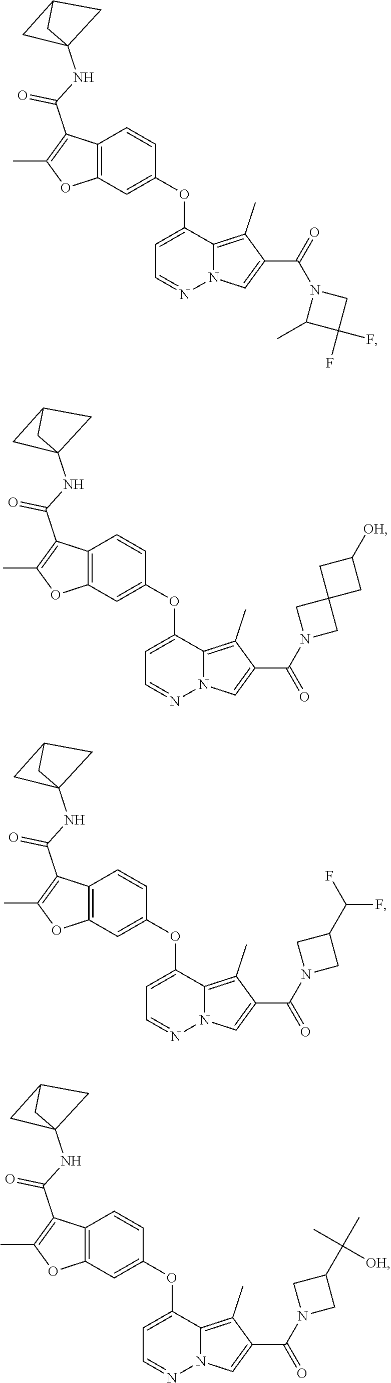 [SMILES: Cc1oc2cc(Oc3ccnn4cc(C(=O)N5CC(F)(F)C5C)c(C)c34)ccc2c1C(=O)NC12CC(C1)C2 ; Cc1oc2cc(Oc3ccnn4cc(C(=O)N5CC6(CC(O)C6)C5)c(C)c34)ccc2c1C(=O)NC12CC(C1)C2 ; Cc1oc2cc(Oc3ccnn4cc(C(=O)N5CC(C(F)F)C5)c(C)c34)ccc2c1C(=O)NC12CC(C1)C2 ; Cc1oc2cc(Oc3ccnn4cc(C(=O)N5CC(C(C)(C)O)C5)c(C)c34)ccc2c1C(=O)NC12CC(C1)C2]

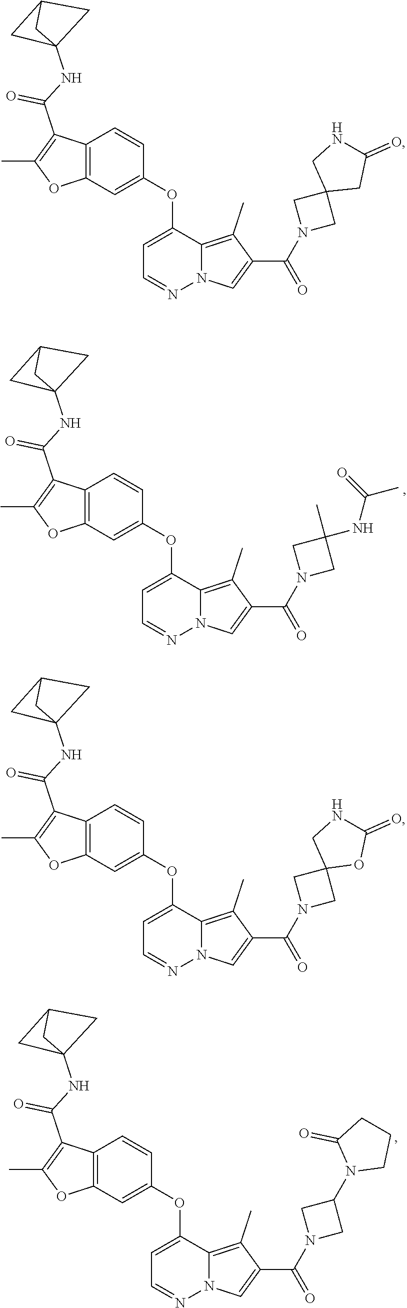

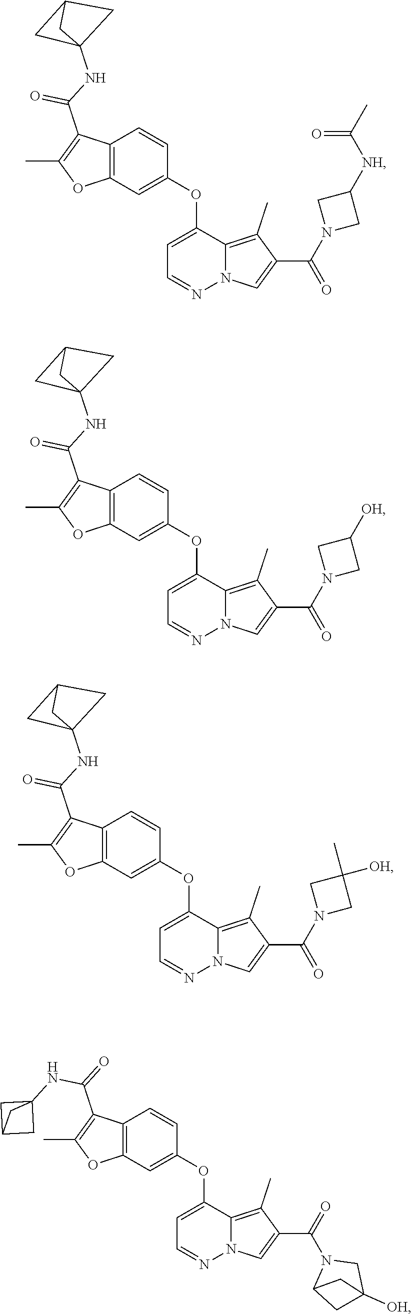

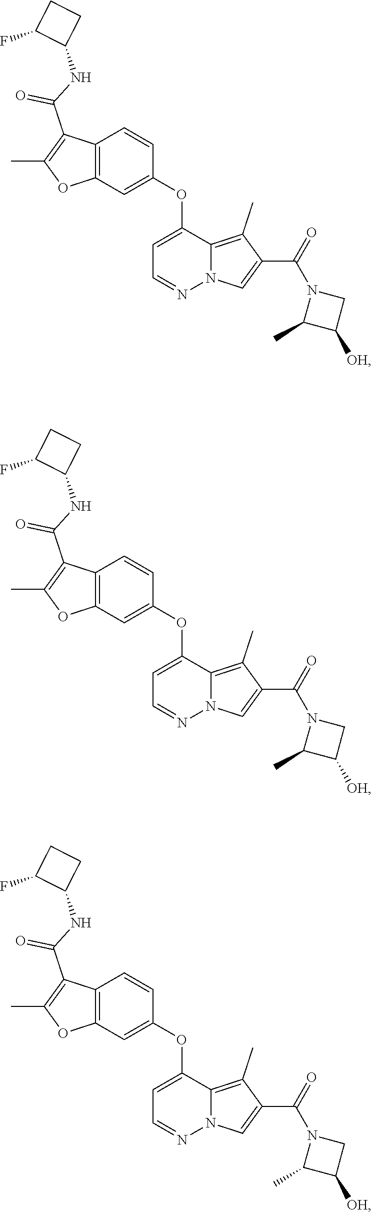 [SMILES: Cc1oc2cc(Oc3ccnn4cc(C(=O)N5C[C@@H](O)[C@H]5C)c(C)c34)ccc2c1C(=O)N[C@H]1CC[C@H]1F ; Cc1oc2cc(Oc3ccnn4cc(C(=O)N5C[C@H](O)[C@H]5C)c(C)c34)ccc2c1C(=O)N[C@H]1CC[C@H]1F ; Cc1oc2cc(Oc3ccnn4cc(C(=O)N5C[C@@H](O)[C@@H]5C)c(C)c34)ccc2c1C(=O)N[C@H]1CC[C@H]1F]

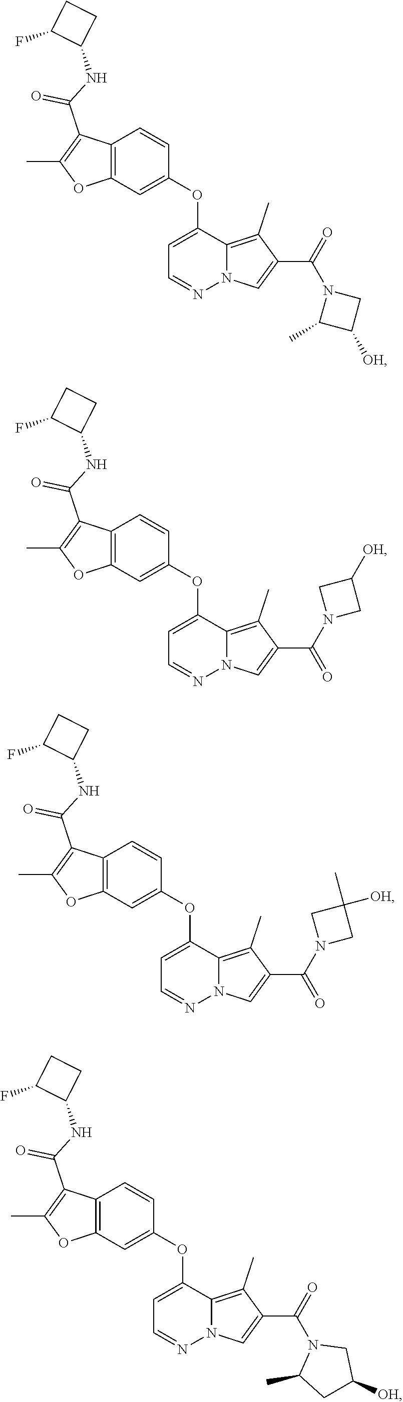 [SMILES: Cc1oc2cc(Oc3ccnn4cc(C(=O)N5C[C@H](O)[C@@H]5C)c(C)c34)ccc2c1C(=O)N[C@H]1CC[C@H]1F ; Cc1oc2cc(Oc3ccnn4cc(C(=O)N5CC(O)C5)c(C)c34)ccc2c1C(=O)N[C@H]1CC[C@H]1F ; Cc1oc2cc(Oc3ccnn4cc(C(=O)N5CC(C)(O)C5)c(C)c34)ccc2c1C(=O)N[C@H]1CC[C@H]1F ; Cc1oc2cc(Oc3ccnn4cc(C(=O)N5C[C@@H](O)C[C@H]5C)c(C)c34)ccc2c1C(=O)N[C@H]1CC[C@H]1F]

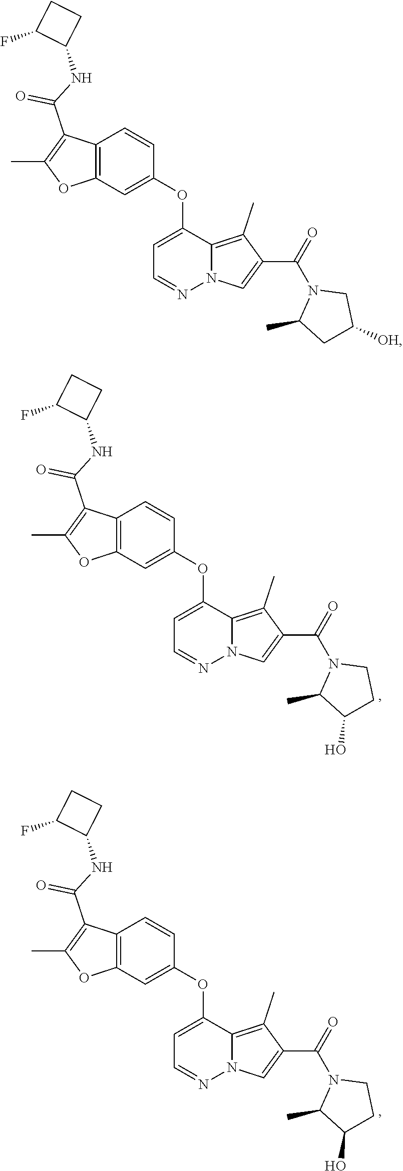 [SMILES: Cc1oc2cc(Oc3ccnn4cc(C(=O)N5C[C@H](O)C[C@H]5C)c(C)c34)ccc2c1C(=O)N[C@H]1CC[C@H]1F ; Cc1oc2cc(Oc3ccnn4cc(C(=O)N5CC[C@H](O)[C@H]5C)c(C)c34)ccc2c1C(=O)N[C@H]1CC[C@H]1F ; Cc1oc2cc(Oc3ccnn4cc(C(=O)N5CC[C@@H](O)[C@H]5C)c(C)c34)ccc2c1C(=O)N[C@H]1CC[C@H]1F]

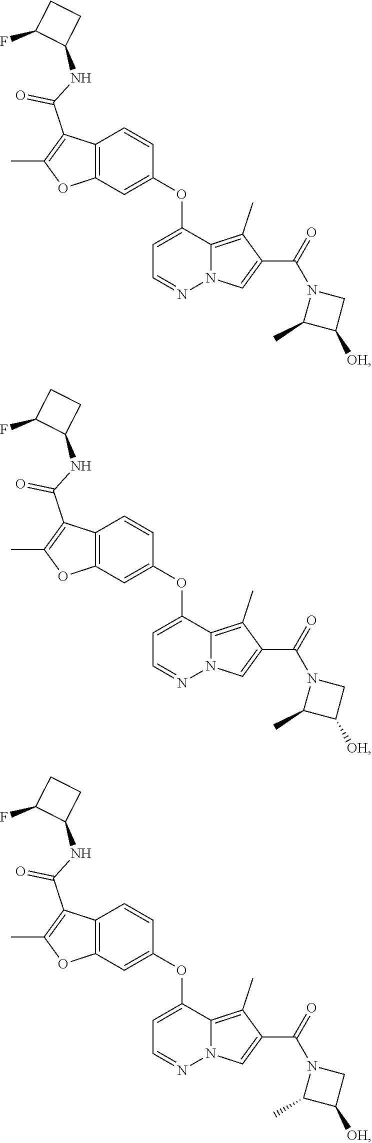 [SMILES: Cc1oc2cc(Oc3ccnn4cc(C(=O)N5C[C@@H](O)[C@H]5C)c(C)c34)ccc2c1C(=O)N[C@@H]1CC[C@@H]1F ; Cc1oc2cc(Oc3ccnn4cc(C(=O)N5C[C@H](O)[C@H]5C)c(C)c34)ccc2c1C(=O)N[C@@H]1CC[C@@H]1F ; Cc1oc2cc(Oc3ccnn4cc(C(=O)N5C[C@@H](O)[C@@H]5C)c(C)c34)ccc2c1C(=O)N[C@@H]1CC[C@@H]1F]

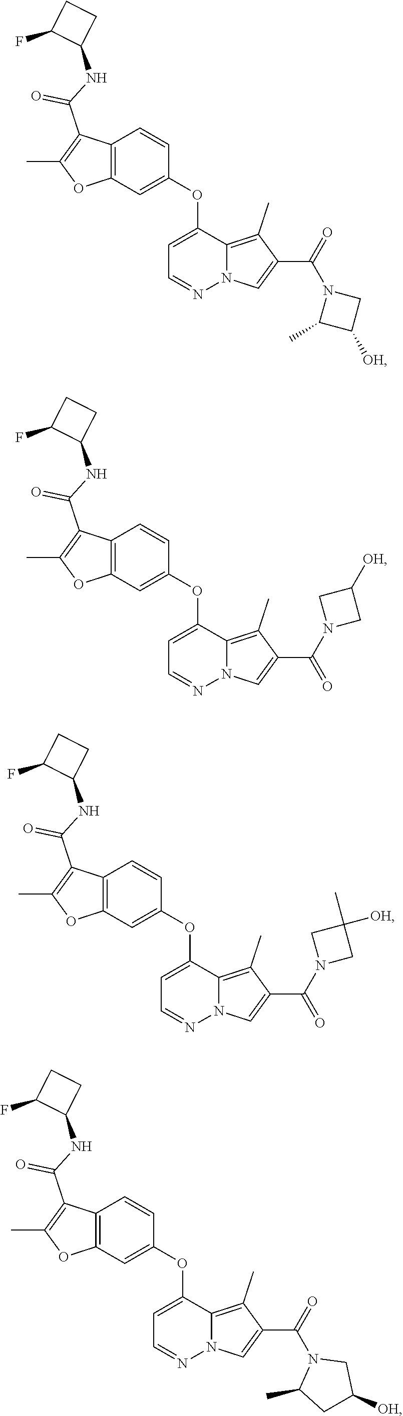 [SMILES: Cc1oc2cc(Oc3ccnn4cc(C(=O)N5C[C@H](O)[C@@H]5C)c(C)c34)ccc2c1C(=O)N[C@@H]1CC[C@@H]1F ; Cc1oc2cc(Oc3ccnn4cc(C(=O)N5CC(O)C5)c(C)c34)ccc2c1C(=O)N[C@@H]1CC[C@@H]1F ; Cc1oc2cc(Oc3ccnn4cc(C(=O)N5CC(C)(O)C5)c(C)c34)ccc2c1C(=O)N[C@@H]1CC[C@@H]1F ; Cc1oc2cc(Oc3ccnn4cc(C(=O)N5C[C@@H](O)C[C@H]5C)c(C)c34)ccc2c1C(=O)N[C@@H]1CC[C@@H]1F]

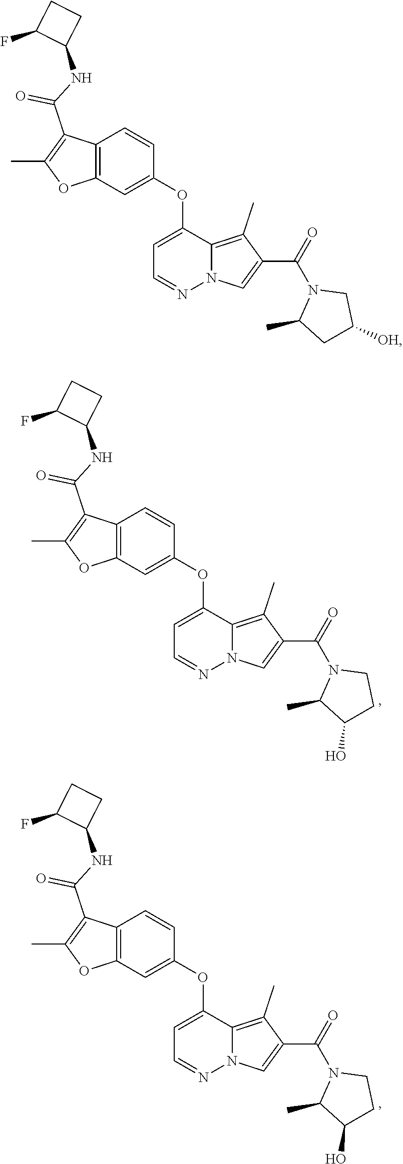 [SMILES: Cc1oc2cc(Oc3ccnn4cc(C(=O)N5C[C@H](O)C[C@H]5C)c(C)c34)ccc2c1C(=O)N[C@@H]1CC[C@@H]1F ; Cc1oc2cc(Oc3ccnn4cc(C(=O)N5CC[C@H](O)[C@H]5C)c(C)c34)ccc2c1C(=O)N[C@@H]1CC[C@@H]1F ; Cc1oc2cc(Oc3ccnn4cc(C(=O)N5CC[C@@H](O)[C@H]5C)c(C)c34)ccc2c1C(=O)N[C@@H]1CC[C@@H]1F]

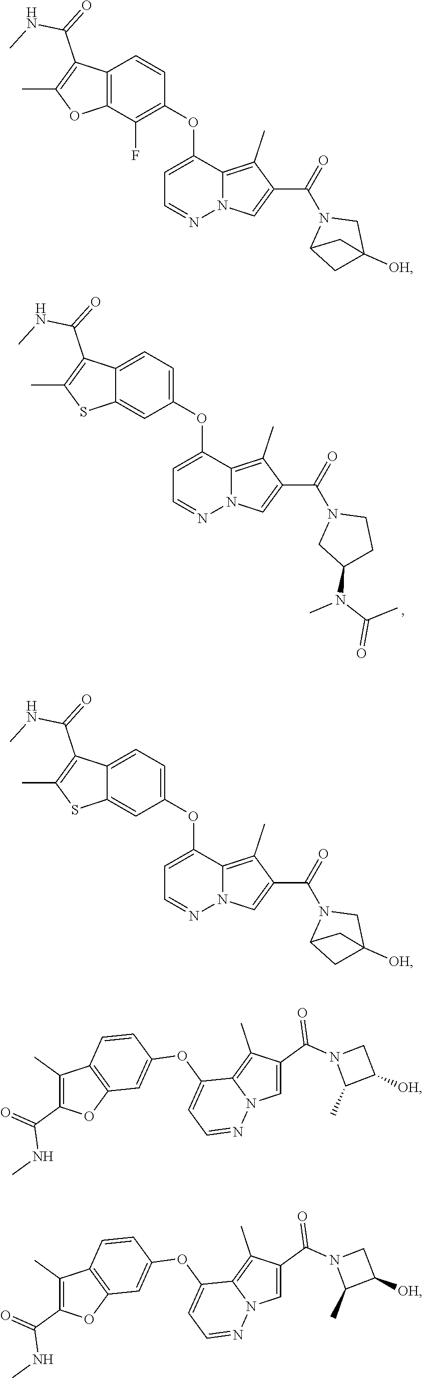

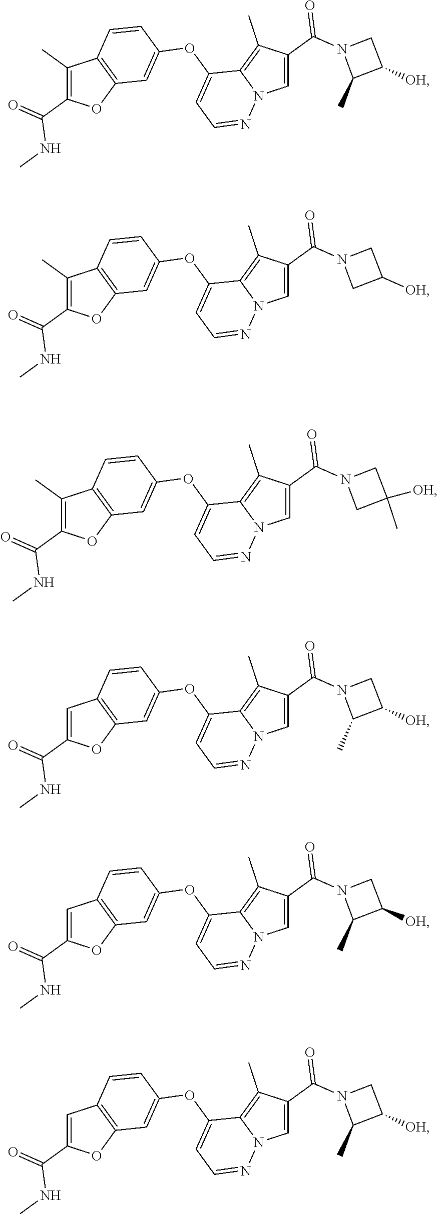 [SMILES: CNC(=O)c1oc2cc(Oc3ccnn4cc(C(=O)N5C[C@H](O)[C@H]5C)c(C)c34)ccc2c1C ; CNC(=O)c1oc2cc(Oc3ccnn4cc(C(=O)N5CC(O)C5)c(C)c34)ccc2c1C ; CNC(=O)c1oc2cc(Oc3ccnn4cc(C(=O)N5CC(C)(O)C5)c(C)c34)ccc2c1C ; CNC(=O)c1cc2ccc(Oc3ccnn4cc(C(=O)N5C[C@H](O)[C@@H]5C)c(C)c34)cc2o1 ; CNC(=O)c1cc2ccc(Oc3ccnn4cc(C(=O)N5C[C@@H](O)[C@H]5C)c(C)c34)cc2o1 ; CNC(=O)c1cc2ccc(Oc3ccnn4cc(C(=O)N5C[C@H](O)[C@H]5C)c(C)c34)cc2o1]

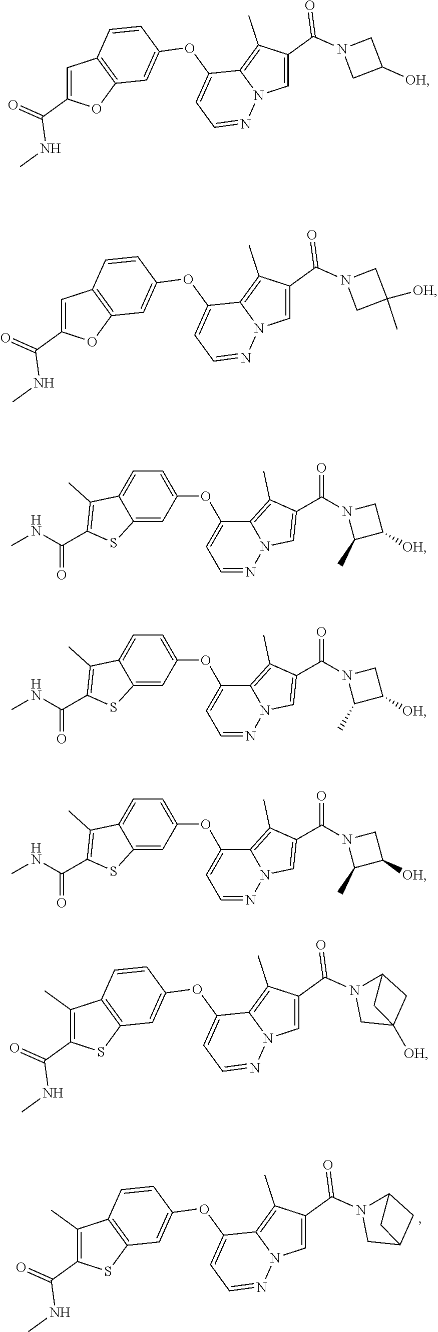

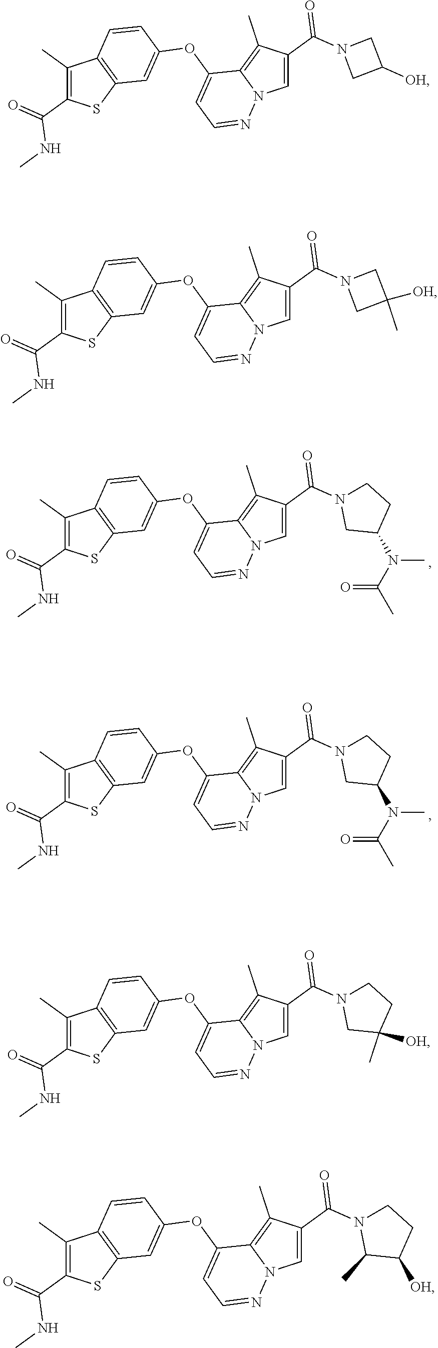 [SMILES: CNC(=O)c1sc2cc(Oc3ccnn4cc(C(=O)N5CC(O)C5)c(C)c34)ccc2c1C ; CNC(=O)c1sc2cc(Oc3ccnn4cc(C(=O)N5CC(C)(O)C5)c(C)c34)ccc2c1C ; CNC(=O)c1sc2cc(Oc3ccnn4cc(C(=O)N5CC[C@H](N(C)C(C)=O)C5)c(C)c34)ccc2c1C ; CNC(=O)c1sc2cc(Oc3ccnn4cc(C(=O)N5CC[C@@H](N(C)C(C)=O)C5)c(C)c34)ccc2c1C ; CNC(=O)c1sc2cc(Oc3ccnn4cc(C(=O)N5CC[C@@](C)(O)C5)c(C)c34)ccc2c1C ; CNC(=O)c1sc2cc(Oc3ccnn4cc(C(=O)N5CC[C@@H](O)[C@H]5C)c(C)c34)ccc2c1C]

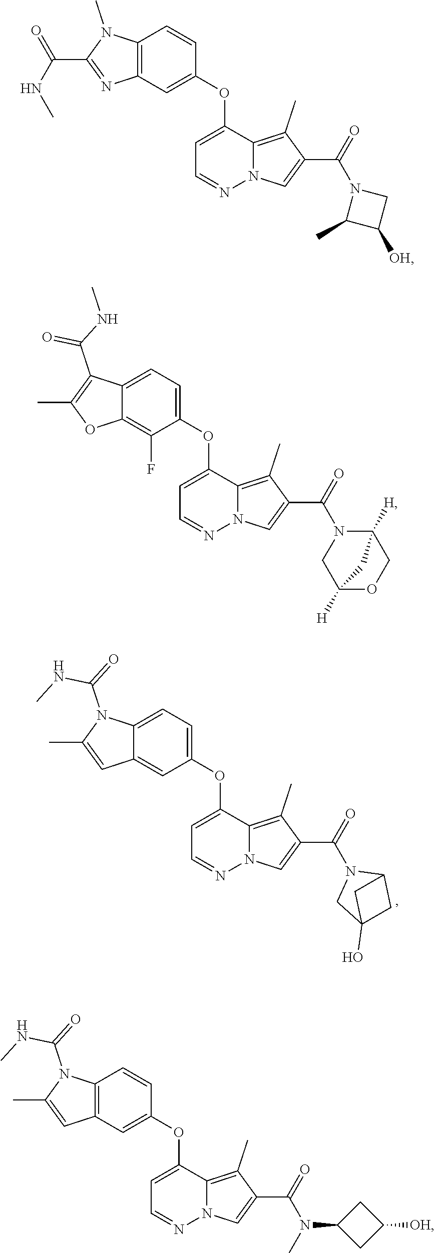

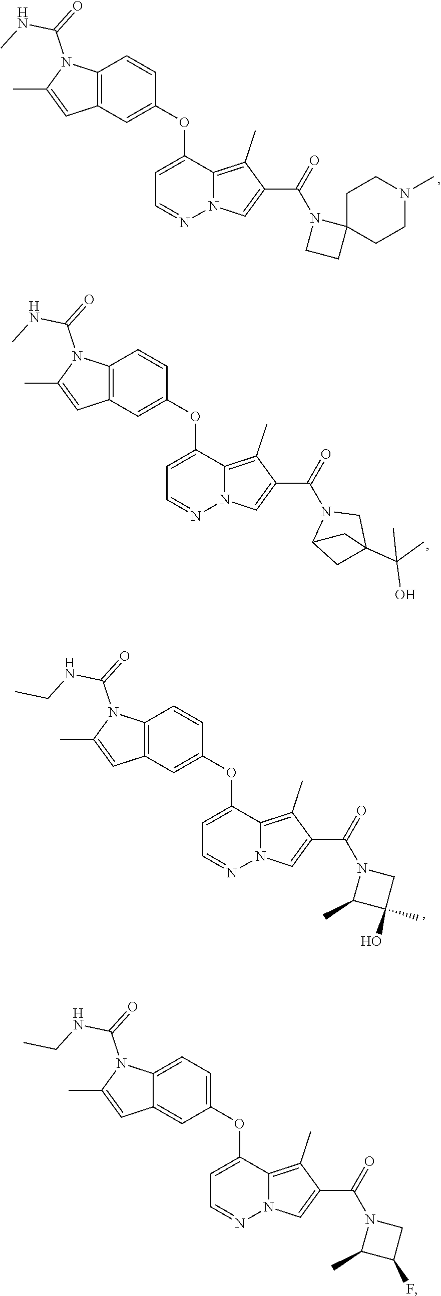

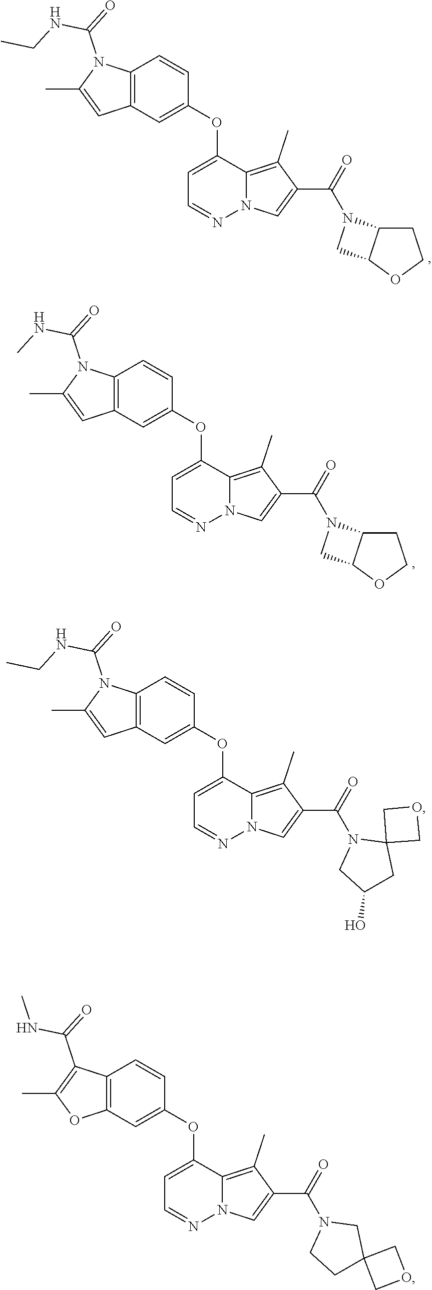 [SMILES: CCNC(=O)n1c(C)cc2cc(Oc3ccnn4cc(C(=O)N5C[C@H]6OCC[C@H]65)c(C)c34)ccc21 ; CNC(=O)n1c(C)cc2cc(Oc3ccnn4cc(C(=O)N5C[C@H]6OCC[C@H]65)c(C)c34)ccc21 ; CCNC(=O)n1c(C)cc2cc(Oc3ccnn4cc(C(=O)N5C[C@@H](O)CC56COC6)c(C)c34)ccc21 ; CNC(=O)c1c(C)oc2cc(Oc3ccnn4cc(C(=O)N5CCC6(COC6)C5)c(C)c34)ccc12]

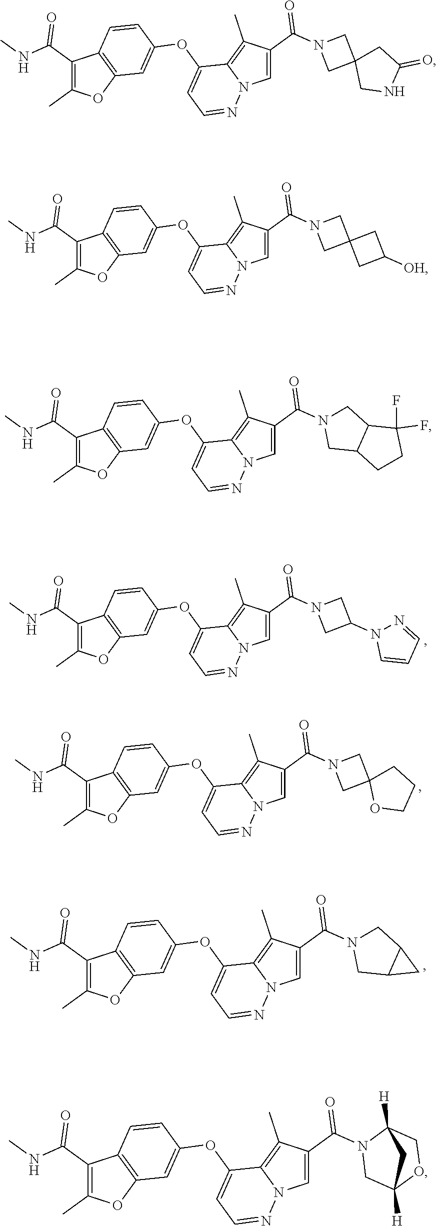

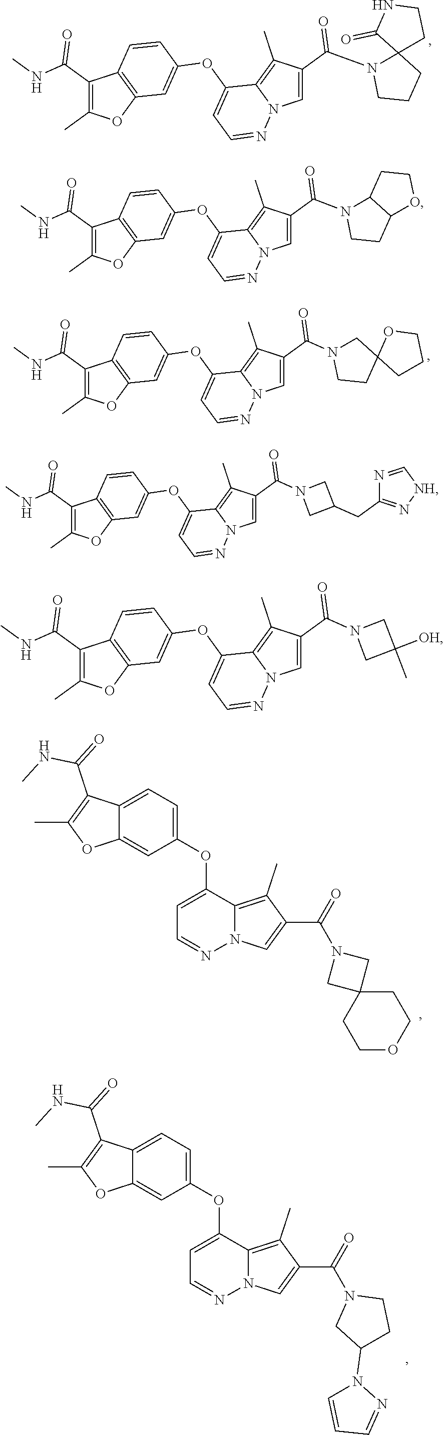

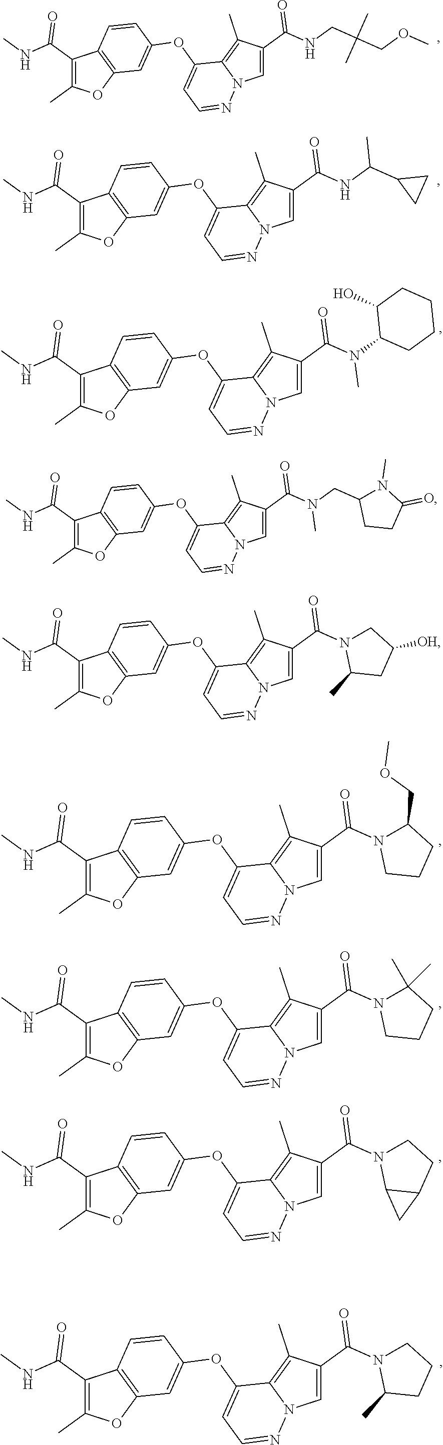

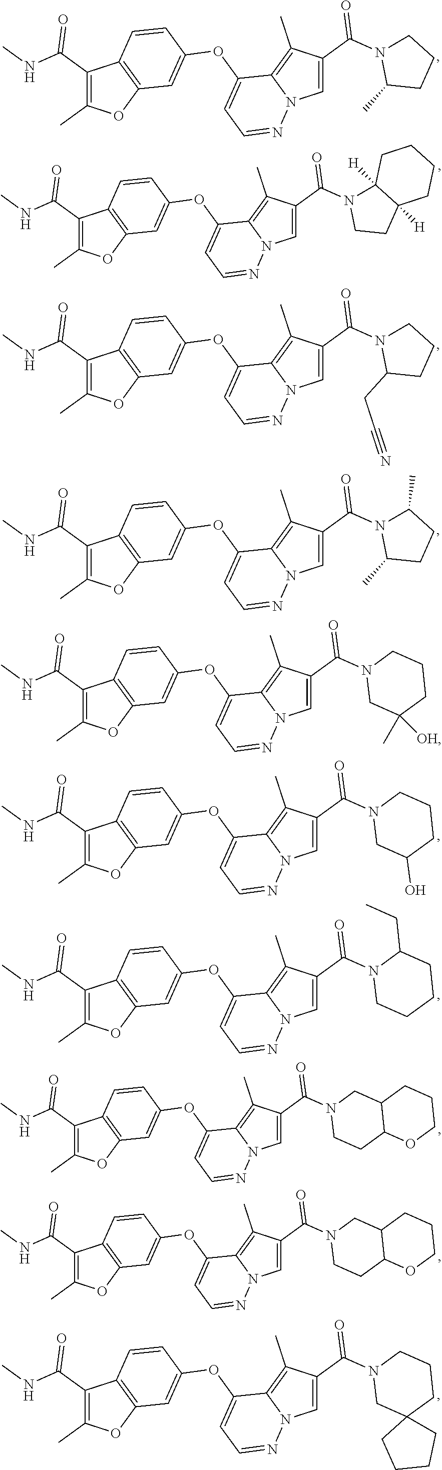

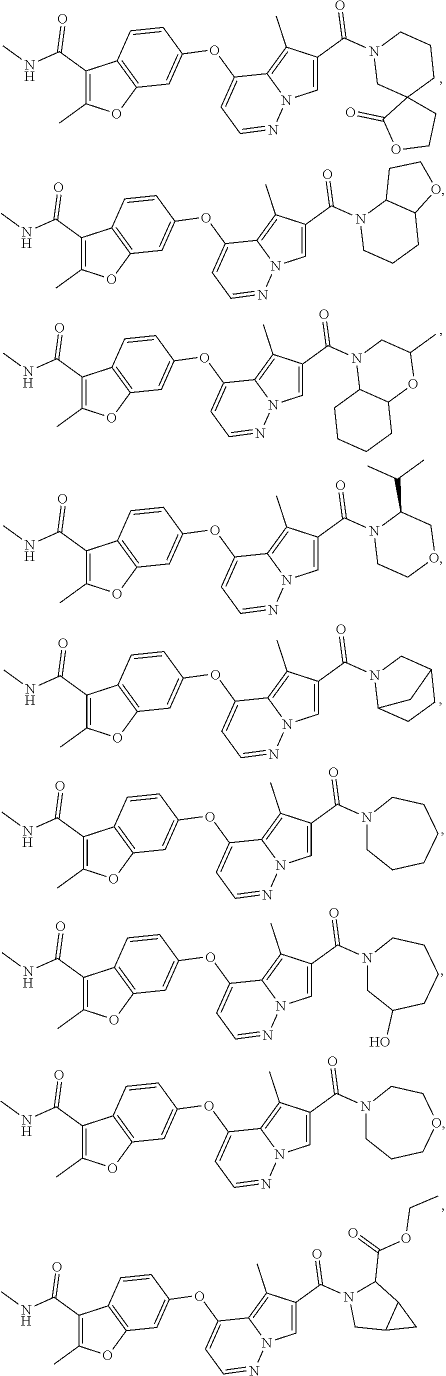

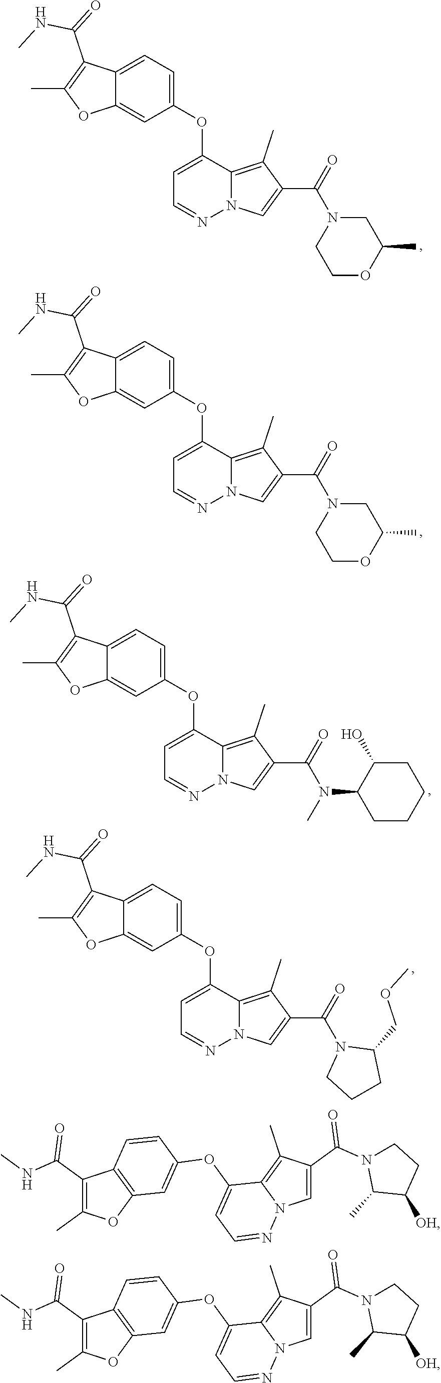

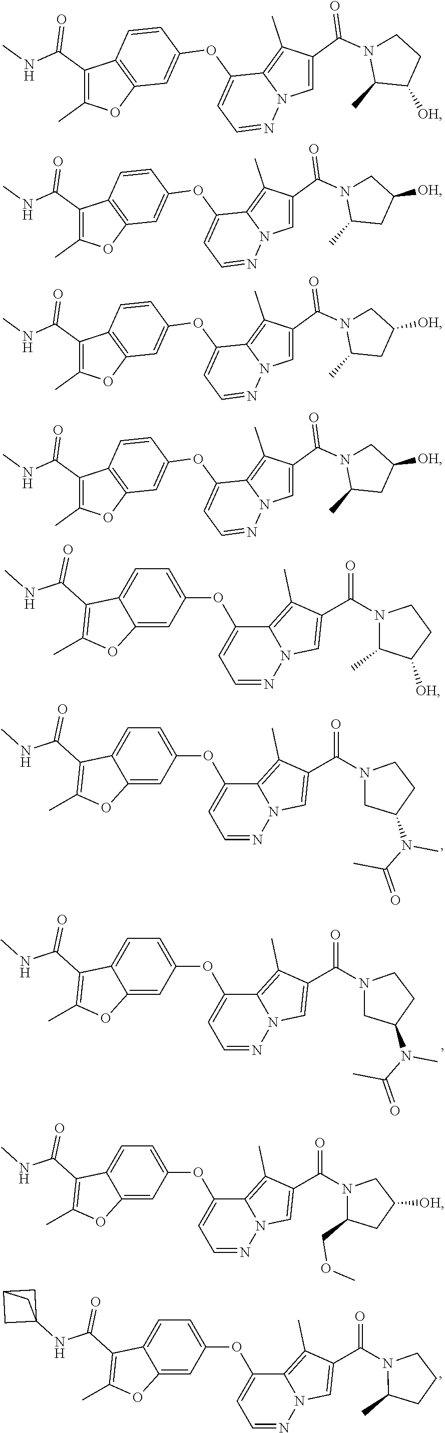

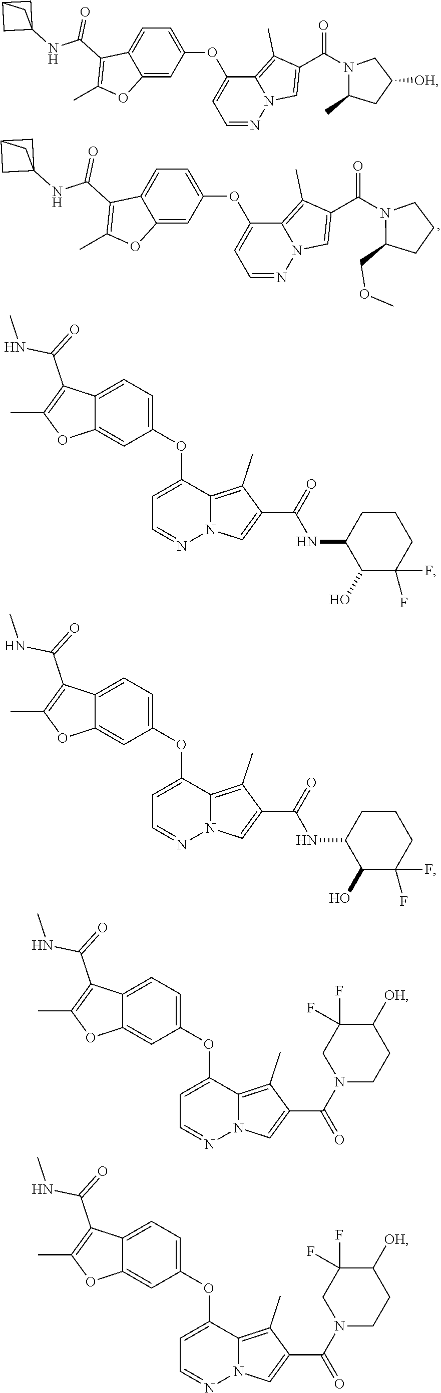 [SMILES: Cc1oc2cc(Oc3ccnn4cc(C(=O)N5C[C@H](O)C[C@H]5C)c(C)c34)ccc2c1C(=O)NC12CC(C1)C2 ; COC[C@@H]1CCCN1C(=O)c1cn2nccc(Oc3ccc4c(C(=O)NC56CC(C5)C6)c(C)oc4c3)c2c1C ; CNC(=O)c1c(C)oc2cc(Oc3ccnn4cc(C(=O)N[C@H]5CCCC(F)(F)[C@@H]5O)c(C)c34)ccc12 ; CNC(=O)c1c(C)oc2cc(Oc3ccnn4cc(C(=O)N[C@@H]5CCCC(F)(F)[C@H]5O)c(C)c34)ccc12 ; CNC(=O)c1c(C)oc2cc(Oc3ccnn4cc(C(=O)N5CCC(O)C(F)(F)C5)c(C)c34)ccc12 ; CNC(=O)c1c(C)oc2cc(Oc3ccnn4cc(C(=O)N5CCC(O)C(F)(F)C5)c(C)c34)ccc12]

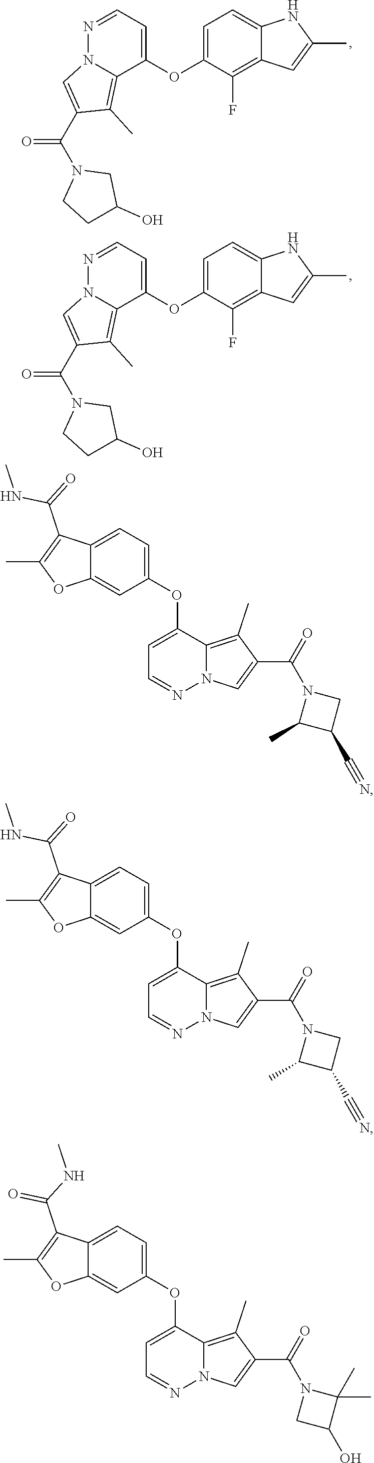

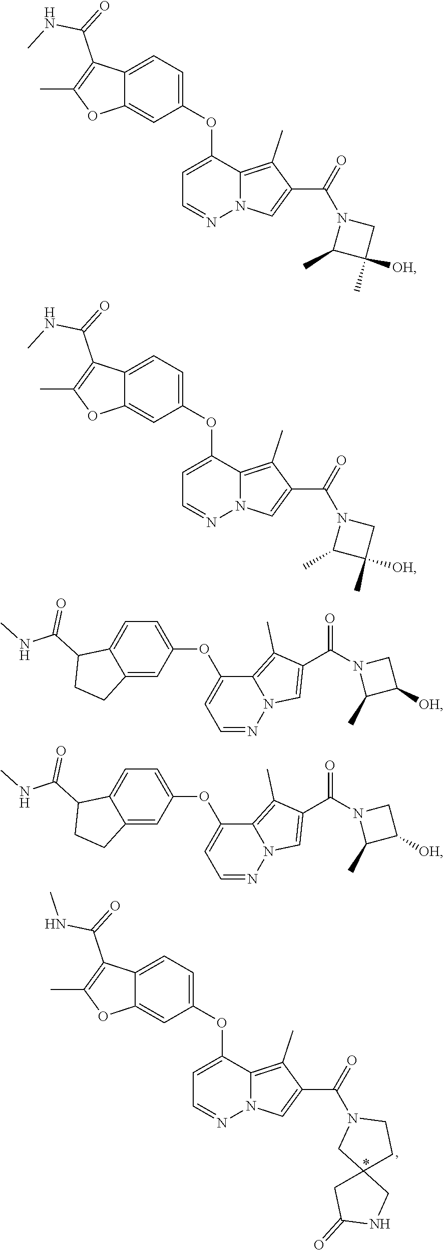

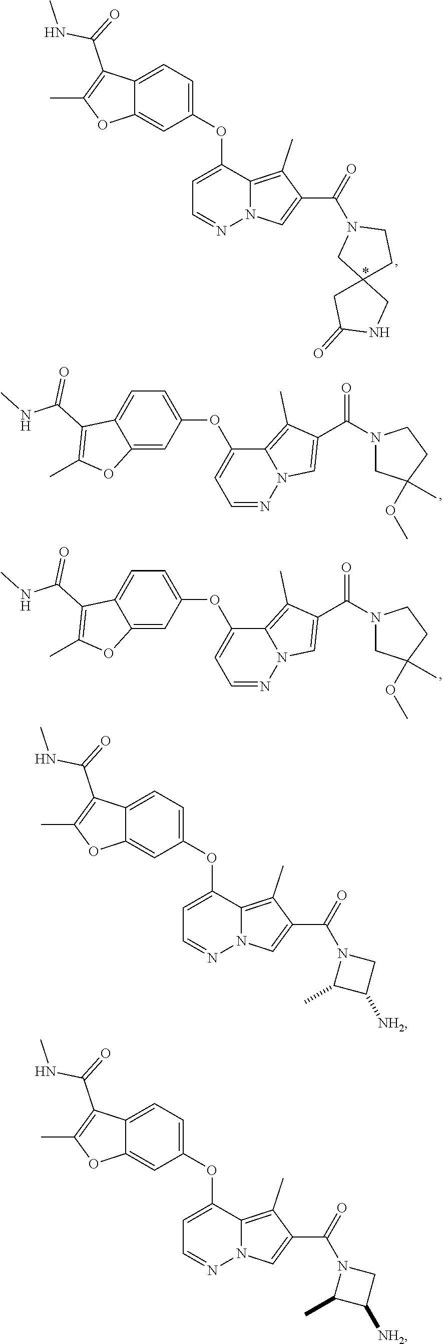

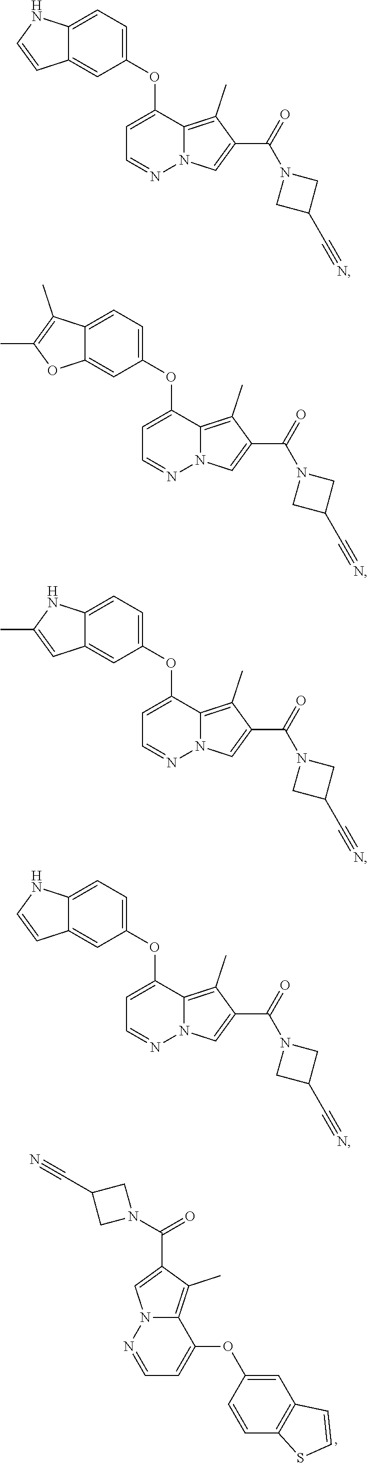 [SMILES: Cc1c(C(=O)N2CC(C#N)C2)cn2nccc(Oc3ccc4[nH]ccc4c3)c12 ; Cc1oc2cc(Oc3ccnn4cc(C(=O)N5CC(C#N)C5)c(C)c34)ccc2c1C ; Cc1cc2cc(Oc3ccnn4cc(C(=O)N5CC(C#N)C5)c(C)c34)ccc2[nH]1 ; Cc1c(C(=O)N2CC(C#N)C2)cn2nccc(Oc3ccc4[nH]ccc4c3)c12 ; Cc1c(C(=O)N2CC(C#N)C2)cn2nccc(Oc3ccc4sccc4c3)c12]

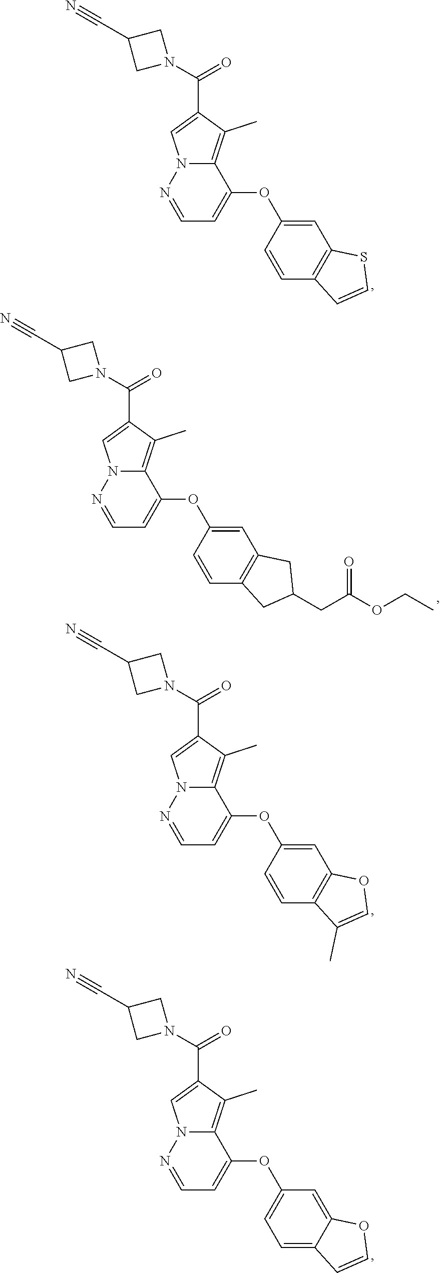 [SMILES: Cc1c(C(=O)N2CC(C#N)C2)cn2nccc(Oc3ccc4ccsc4c3)c12 ; CCOC(=O)CC1Cc2ccc(Oc3ccnn4cc(C(=O)N5CC(C#N)C5)c(C)c34)cc2C1 ; Cc1coc2cc(Oc3ccnn4cc(C(=O)N5CC(C#N)C5)c(C)c34)ccc12 ; Cc1c(C(=O)N2CC(C#N)C2)cn2nccc(Oc3ccc4ccoc4c3)c12]

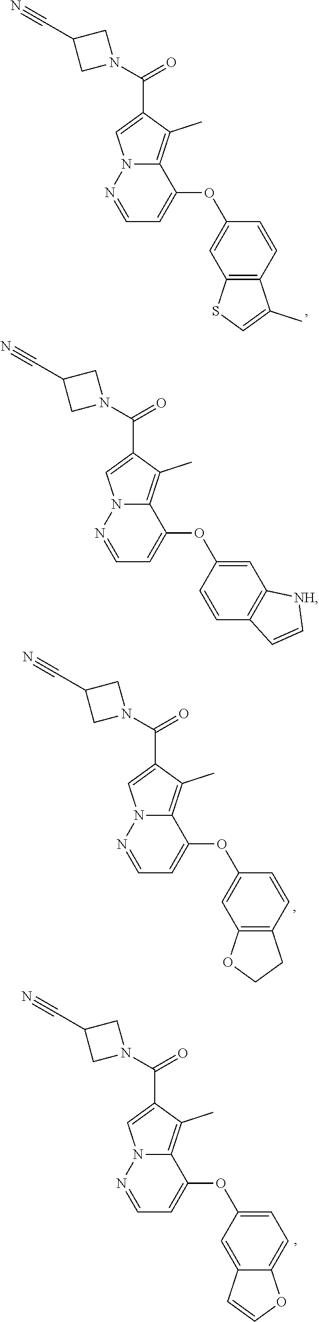 [SMILES: Cc1csc2cc(Oc3ccnn4cc(C(=O)N5CC(C#N)C5)c(C)c34)ccc12 ; Cc1c(C(=O)N2CC(C#N)C2)cn2nccc(Oc3ccc4cc[nH]c4c3)c12 ; Cc1c(C(=O)N2CC(C#N)C2)cn2nccc(Oc3ccc4c(c3)OCC4)c12 ; Cc1c(C(=O)N2CC(C#N)C2)cn2nccc(Oc3ccc4occc4c3)c12]

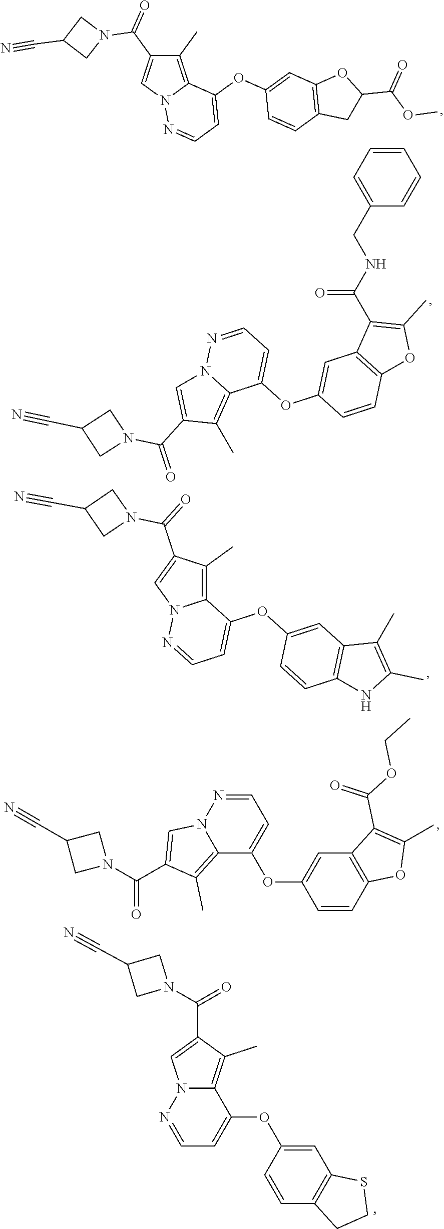

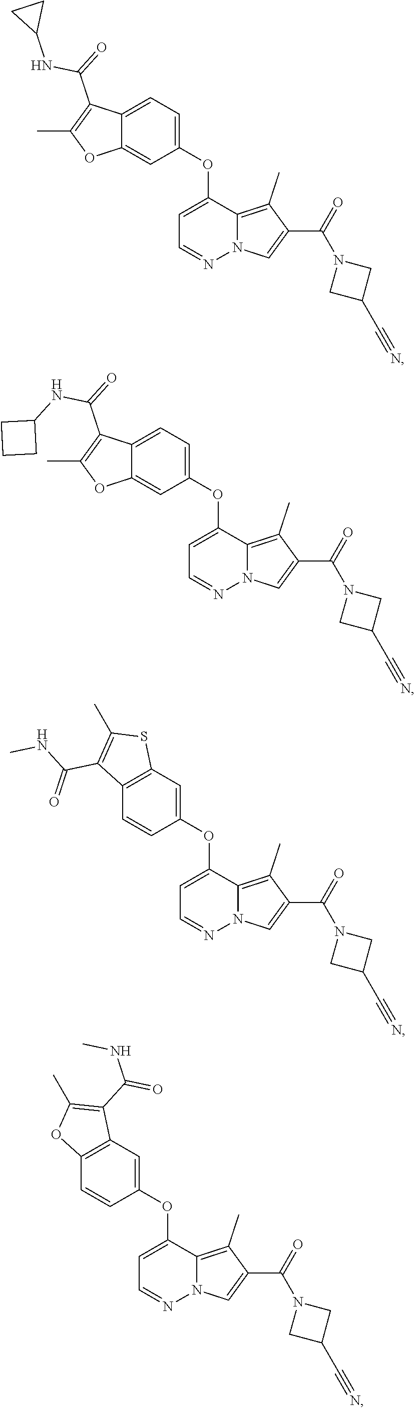 [SMILES: Cc1oc2cc(Oc3ccnn4cc(C(=O)N5CC(C#N)C5)c(C)c34)ccc2c1C(=O)NC1CC1 ; Cc1oc2cc(Oc3ccnn4cc(C(=O)N5CC(C#N)C5)c(C)c34)ccc2c1C(=O)NC1CCC1 ; CNC(=O)c1c(C)sc2cc(Oc3ccnn4cc(C(=O)N5CC(C#N)C5)c(C)c34)ccc12 ; CNC(=O)c1c(C)oc2ccc(Oc3ccnn4cc(C(=O)N5CC(C#N)C5)c(C)c34)cc12]

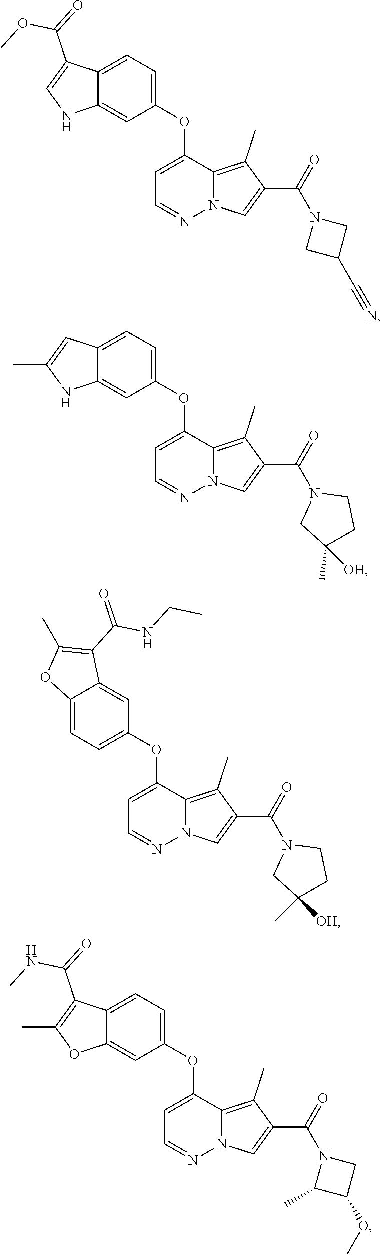 [SMILES: COC(=O)c1c[nH]c2cc(Oc3ccnn4cc(C(=O)N5CC(C#N)C5)c(C)c34)ccc12 ; Cc1cc2ccc(Oc3ccnn4cc(C(=O)N5CC[C@@](C)(O)C5)c(C)c34)cc2[nH]1 ; CCNC(=O)c1c(C)oc2ccc(Oc3ccnn4cc(C(=O)N5CC[C@@](C)(O)C5)c(C)c34)cc12 ; CNC(=O)c1c(C)oc2cc(Oc3ccnn4cc(C(=O)N5C[C@H](OC)[C@@H]5C)c(C)c34)ccc12]

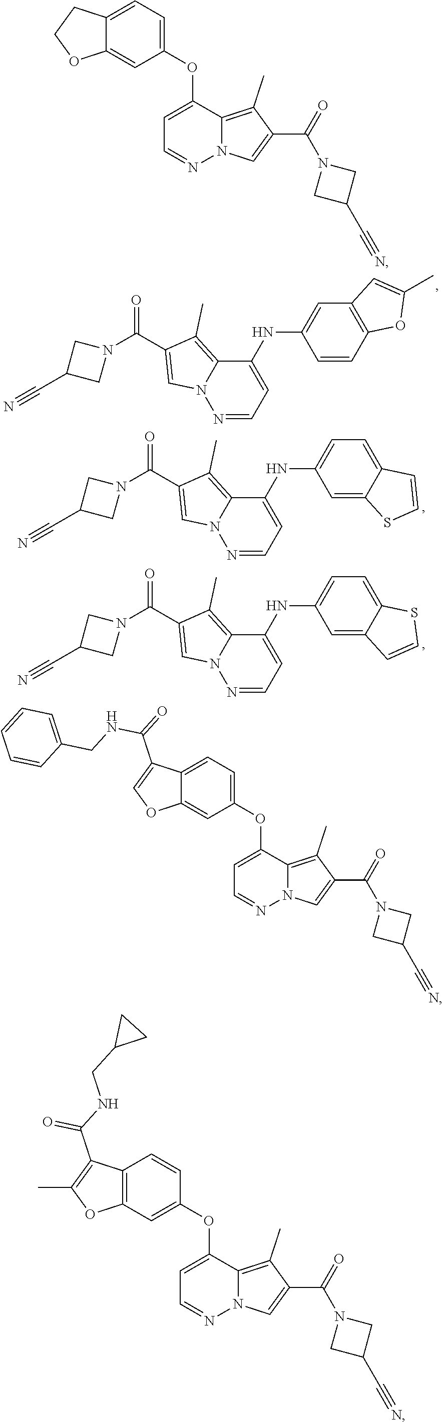

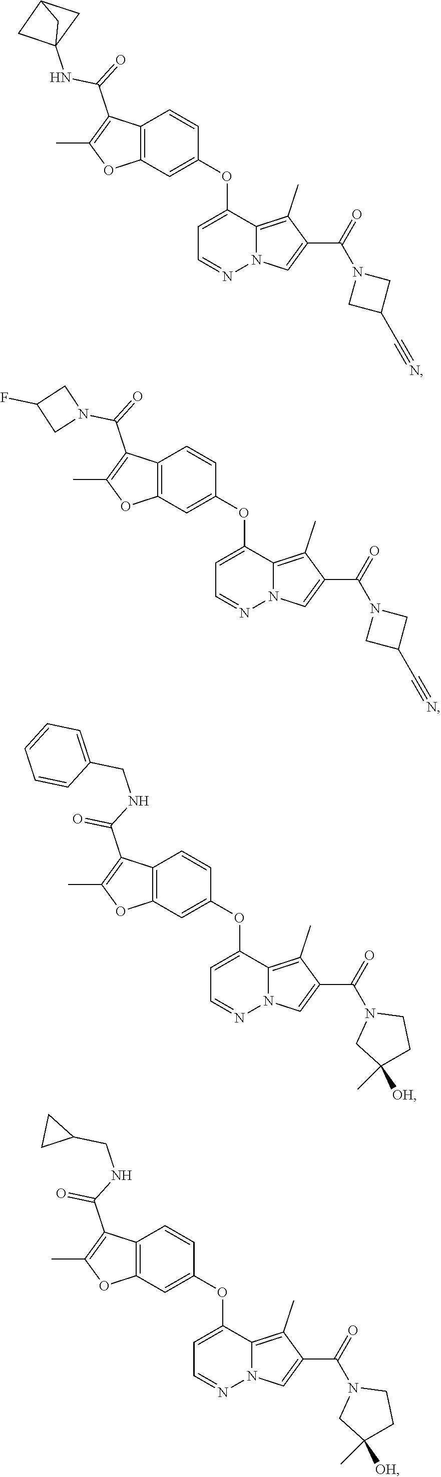 [SMILES: Cc1oc2cc(Oc3ccnn4cc(C(=O)N5CC(C#N)C5)c(C)c34)ccc2c1C(=O)NC12CC(C1)C2 ; Cc1oc2cc(Oc3ccnn4cc(C(=O)N5CC(C#N)C5)c(C)c34)ccc2c1C(=O)N1CC(F)C1 ; Cc1oc2cc(Oc3ccnn4cc(C(=O)N5CC[C@@](C)(O)C5)c(C)c34)ccc2c1C(=O)NCc1ccccc1 ; Cc1oc2cc(Oc3ccnn4cc(C(=O)N5CC[C@@](C)(O)C5)c(C)c34)ccc2c1C(=O)NCC1CC1]

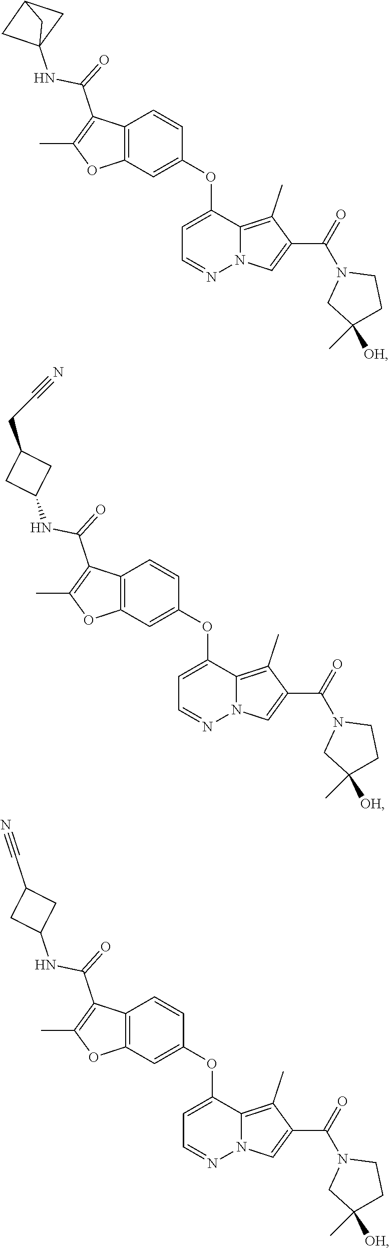 [SMILES: Cc1oc2cc(Oc3ccnn4cc(C(=O)N5CC[C@@](C)(O)C5)c(C)c34)ccc2c1C(=O)NC12CC(C1)C2 ; Cc1oc2cc(Oc3ccnn4cc(C(=O)N5CC[C@@](C)(O)C5)c(C)c34)ccc2c1C(=O)N[C@H]1C[C@H](CC#N)C1 ; Cc1oc2cc(Oc3ccnn4cc(C(=O)N5CC[C@@](C)(O)C5)c(C)c34)ccc2c1C(=O)NC1CC(C#N)C1]

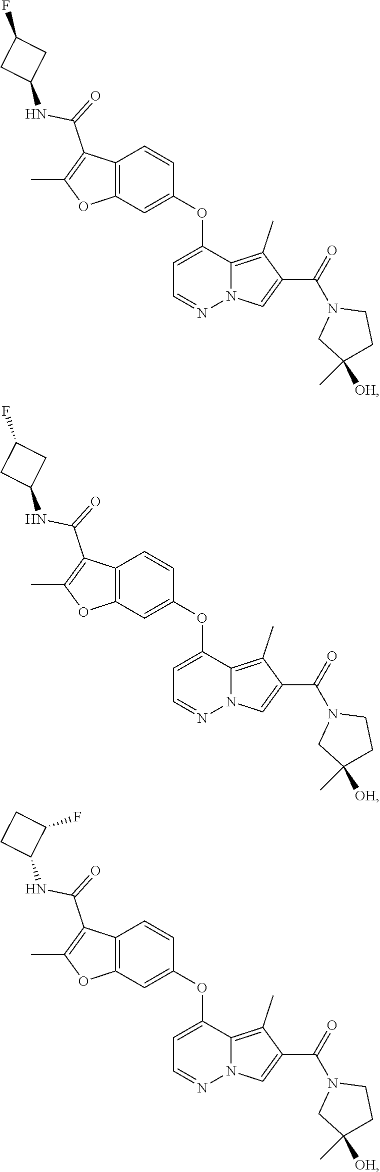 [SMILES: Cc1oc2cc(Oc3ccnn4cc(C(=O)N5CC[C@@](C)(O)C5)c(C)c34)ccc2c1C(=O)N[C@H]1C[C@@H](F)C1 ; Cc1oc2cc(Oc3ccnn4cc(C(=O)N5CC[C@@](C)(O)C5)c(C)c34)ccc2c1C(=O)N[C@H]1C[C@H](F)C1 ; Cc1oc2cc(Oc3ccnn4cc(C(=O)N5CC[C@@](C)(O)C5)c(C)c34)ccc2c1C(=O)N[C@@H]1CC[C@@H]1F]

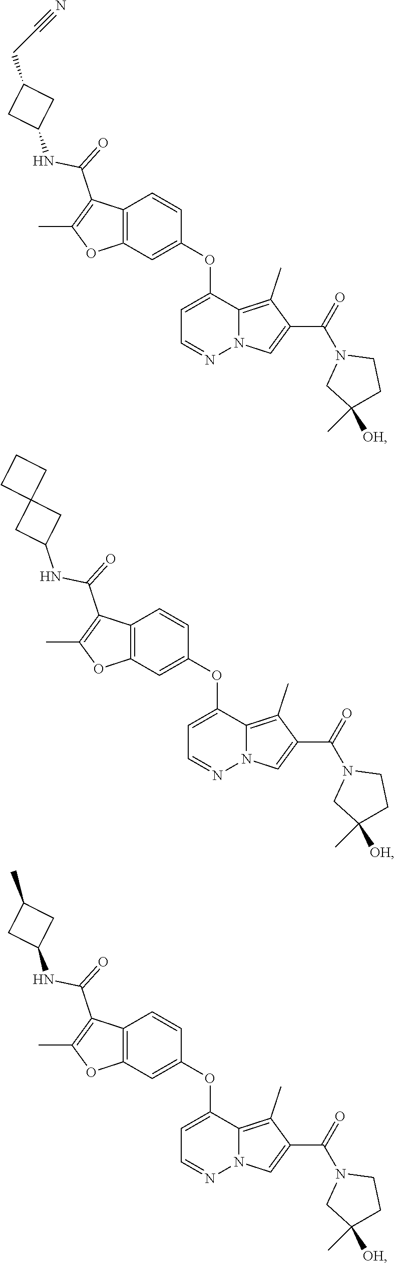 [SMILES: Cc1oc2cc(Oc3ccnn4cc(C(=O)N5CC[C@@](C)(O)C5)c(C)c34)ccc2c1C(=O)N[C@H]1C[C@@H](CC#N)C1 ; Cc1oc2cc(Oc3ccnn4cc(C(=O)N5CC[C@@](C)(O)C5)c(C)c34)ccc2c1C(=O)NC1CC2(CCC2)C1 ; Cc1oc2cc(Oc3ccnn4cc(C(=O)N5CC[C@@](C)(O)C5)c(C)c34)ccc2c1C(=O)N[C@H]1C[C@@H](C)C1]

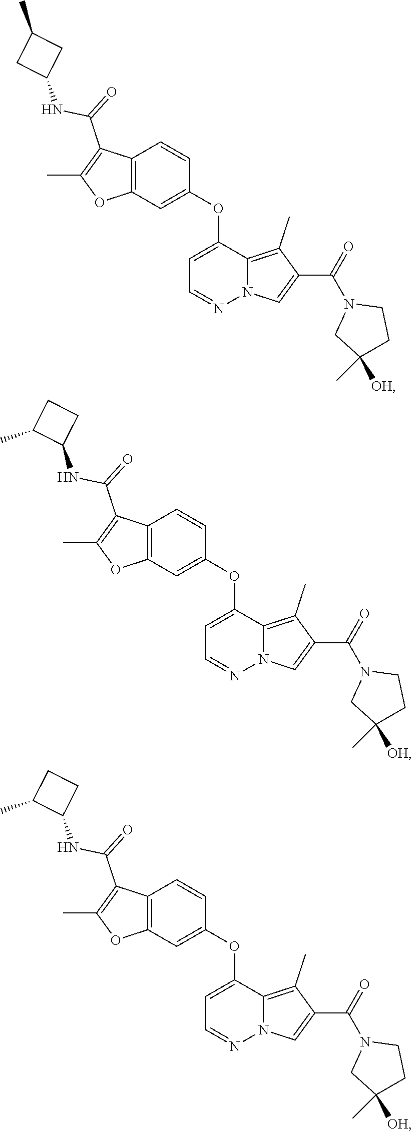 [SMILES: Cc1oc2cc(Oc3ccnn4cc(C(=O)N5CC[C@@](C)(O)C5)c(C)c34)ccc2c1C(=O)N[C@H]1C[C@H](C)C1 ; Cc1oc2cc(Oc3ccnn4cc(C(=O)N5CC[C@@](C)(O)C5)c(C)c34)ccc2c1C(=O)N[C@@H]1CC[C@H]1C ; Cc1oc2cc(Oc3ccnn4cc(C(=O)N5CC[C@@](C)(O)C5)c(C)c34)ccc2c1C(=O)N[C@H]1CC[C@H]1C]

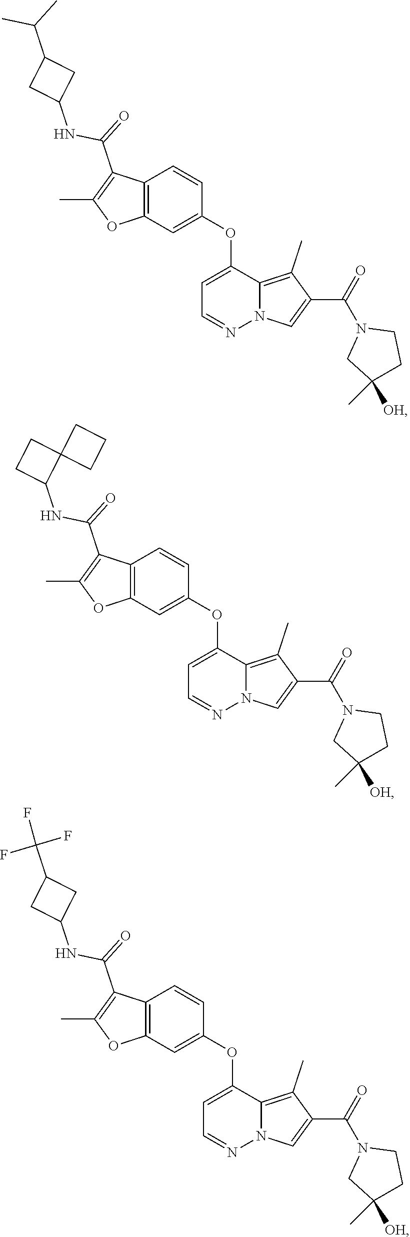 [SMILES: Cc1oc2cc(Oc3ccnn4cc(C(=O)N5CC[C@@](C)(O)C5)c(C)c34)ccc2c1C(=O)NC1CC(C(C)C)C1 ; Cc1oc2cc(Oc3ccnn4cc(C(=O)N5CC[C@@](C)(O)C5)c(C)c34)ccc2c1C(=O)NC1CCC12CCC2 ; Cc1oc2cc(Oc3ccnn4cc(C(=O)N5CC[C@@](C)(O)C5)c(C)c34)ccc2c1C(=O)NC1CC(C(F)(F)F)C1]

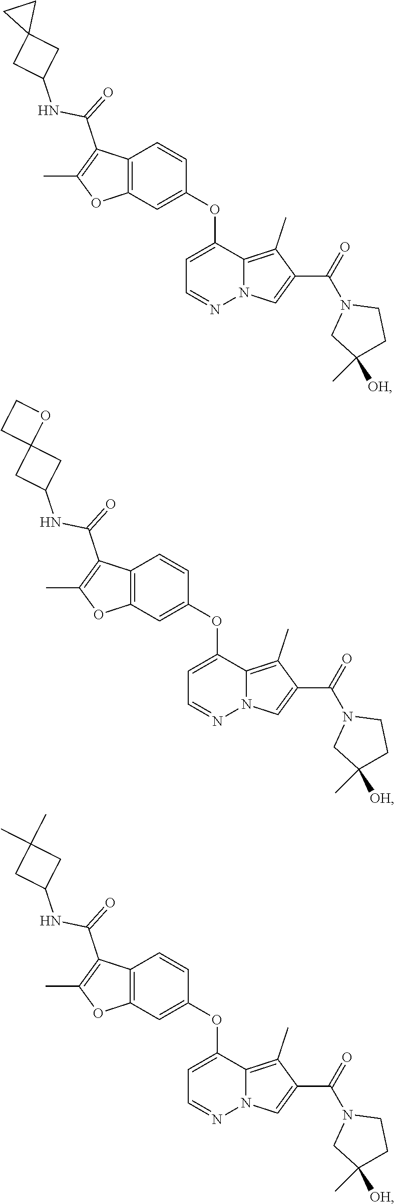 [SMILES: Cc1oc2cc(Oc3ccnn4cc(C(=O)N5CC[C@@](C)(O)C5)c(C)c34)ccc2c1C(=O)NC1CC2(CC2)C1 ; Cc1oc2cc(Oc3ccnn4cc(C(=O)N5CC[C@@](C)(O)C5)c(C)c34)ccc2c1C(=O)NC1CC2(CCO2)C1 ; Cc1oc2cc(Oc3ccnn4cc(C(=O)N5CC[C@@](C)(O)C5)c(C)c34)ccc2c1C(=O)NC1CC(C)(C)C1]

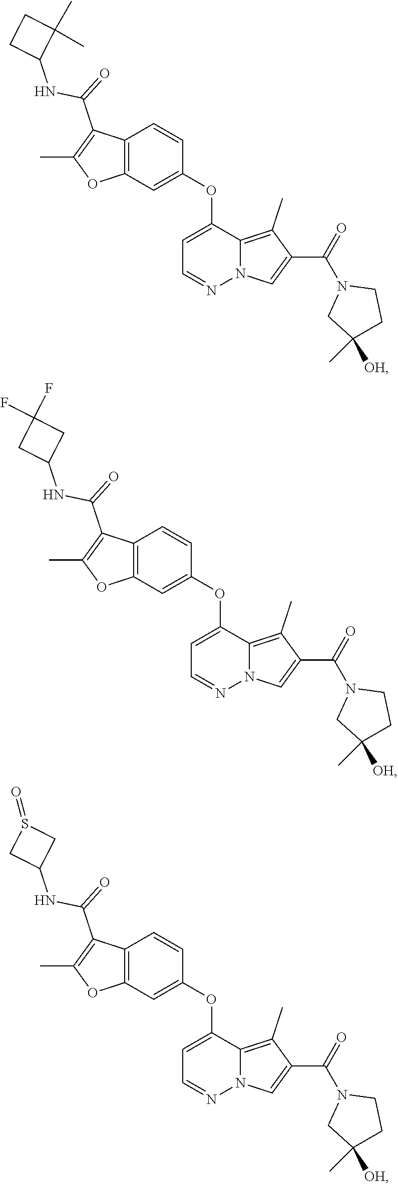 [SMILES: Cc1oc2cc(Oc3ccnn4cc(C(=O)N5CC[C@@](C)(O)C5)c(C)c34)ccc2c1C(=O)NC1CCC1(C)C ; Cc1oc2cc(Oc3ccnn4cc(C(=O)N5CC[C@@](C)(O)C5)c(C)c34)ccc2c1C(=O)NC1CC(F)(F)C1 ; Cc1oc2cc(Oc3ccnn4cc(C(=O)N5CC[C@@](C)(O)C5)c(C)c34)ccc2c1C(=O)NC1CS(=O)C1]

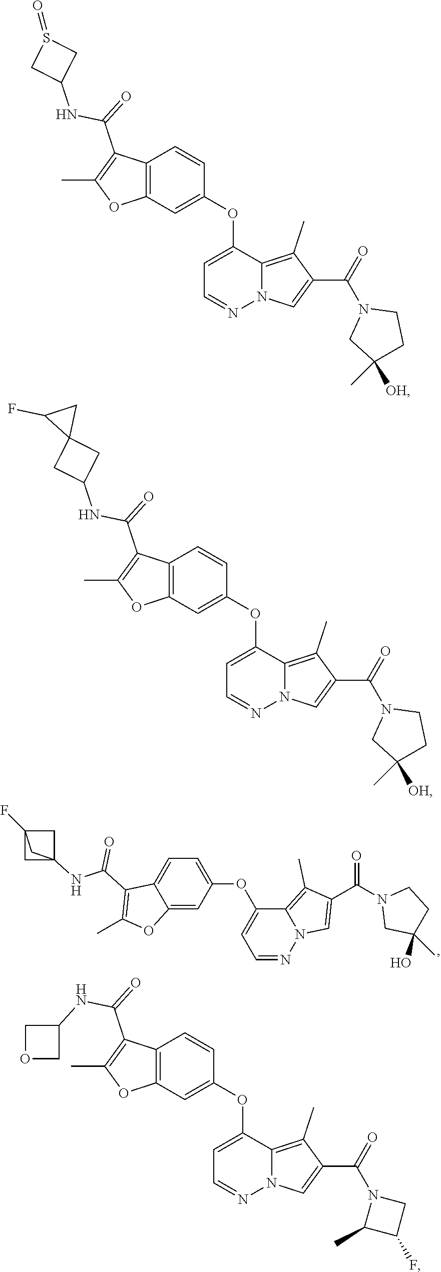 [SMILES: Cc1oc2cc(Oc3ccnn4cc(C(=O)N5CC[C@@](C)(O)C5)c(C)c34)ccc2c1C(=O)NC1CS(=O)C1 ; Cc1oc2cc(Oc3ccnn4cc(C(=O)N5CC[C@@](C)(O)C5)c(C)c34)ccc2c1C(=O)NC1CC2(C1)CC2F ; Cc1oc2cc(Oc3ccnn4cc(C(=O)N5CC[C@@](C)(O)C5)c(C)c34)ccc2c1C(=O)NC12CC(F)(C1)C2 ; Cc1oc2cc(Oc3ccnn4cc(C(=O)N5C[C@H](F)[C@H]5C)c(C)c34)ccc2c1C(=O)NC1COC1]

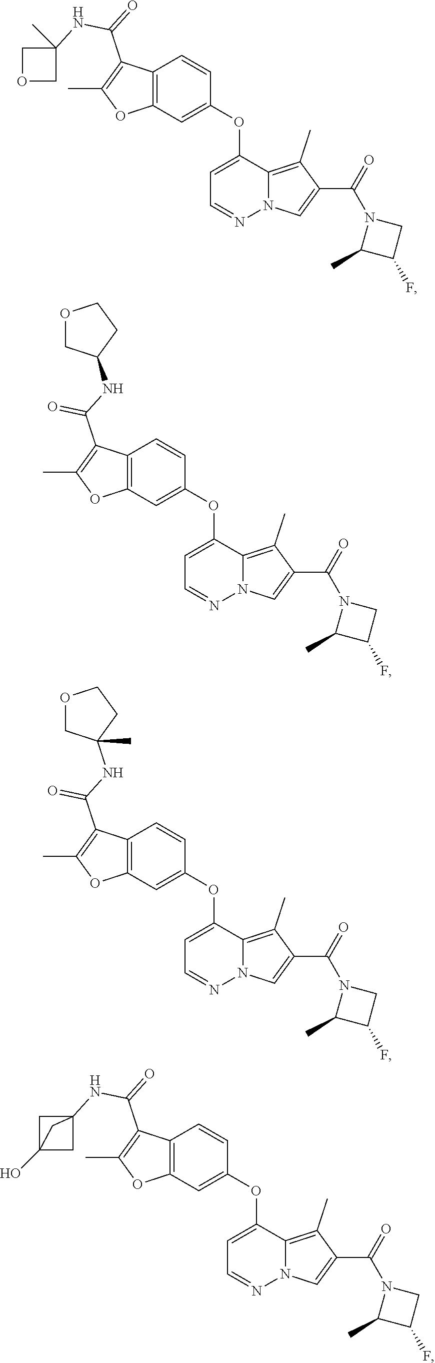 [SMILES: Cc1oc2cc(Oc3ccnn4cc(C(=O)N5C[C@H](F)[C@H]5C)c(C)c34)ccc2c1C(=O)NC1(C)COC1 ; Cc1oc2cc(Oc3ccnn4cc(C(=O)N5C[C@H](F)[C@H]5C)c(C)c34)ccc2c1C(=O)N[C@@H]1CCOC1 ; Cc1oc2cc(Oc3ccnn4cc(C(=O)N5C[C@H](F)[C@H]5C)c(C)c34)ccc2c1C(=O)N[C@@]1(C)CCOC1 ; Cc1oc2cc(Oc3ccnn4cc(C(=O)N5C[C@H](F)[C@H]5C)c(C)c34)ccc2c1C(=O)NC12CC(O)(C1)C2]

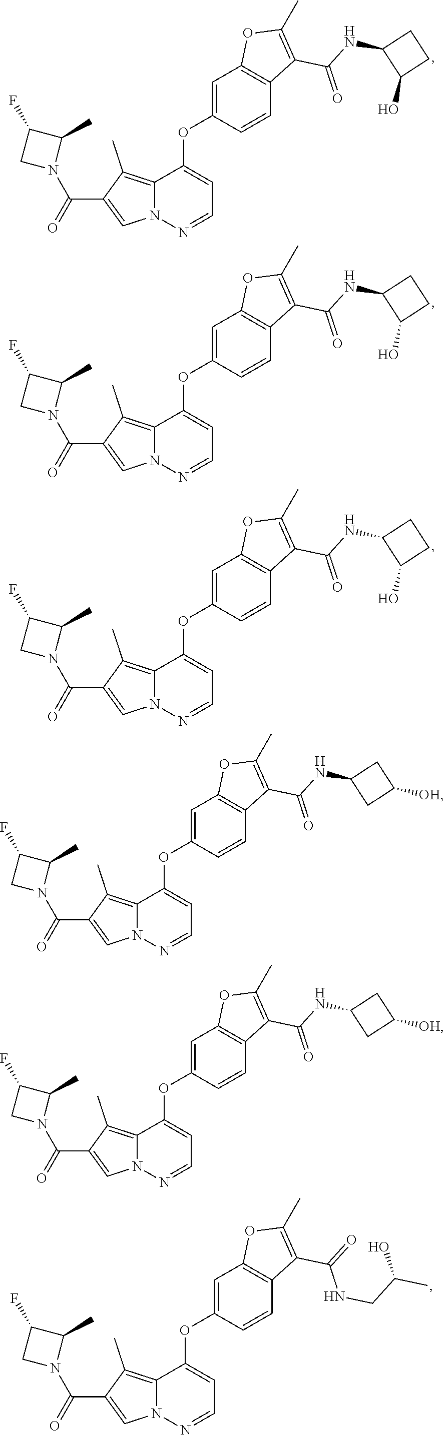 [SMILES: Cc1oc2cc(Oc3ccnn4cc(C(=O)N5C[C@H](F)[C@H]5C)c(C)c34)ccc2c1C(=O)N[C@H]1CC[C@H]1O ; Cc1oc2cc(Oc3ccnn4cc(C(=O)N5C[C@H](F)[C@H]5C)c(C)c34)ccc2c1C(=O)N[C@H]1CC[C@@H]1O ; Cc1oc2cc(Oc3ccnn4cc(C(=O)N5C[C@H](F)[C@H]5C)c(C)c34)ccc2c1C(=O)N[C@@H]1CC[C@@H]1O ; Cc1oc2cc(Oc3ccnn4cc(C(=O)N5C[C@H](F)[C@H]5C)c(C)c34)ccc2c1C(=O)N[C@H]1C[C@H](O)C1 ; Cc1oc2cc(Oc3ccnn4cc(C(=O)N5C[C@H](F)[C@H]5C)c(C)c34)ccc2c1C(=O)N[C@H]1C[C@@H](O)C1 ; Cc1oc2cc(Oc3ccnn4cc(C(=O)N5C[C@H](F)[C@H]5C)c(C)c34)ccc2c1C(=O)NC[C@@H](C)O]

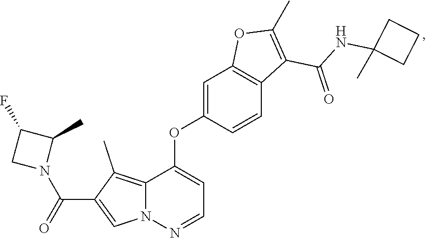 [SMILES: Cc1oc2cc(Oc3ccnn4cc(C(=O)N5C[C@H](F)[C@H]5C)c(C)c34)ccc2c1C(=O)NC1(C)CCC1]

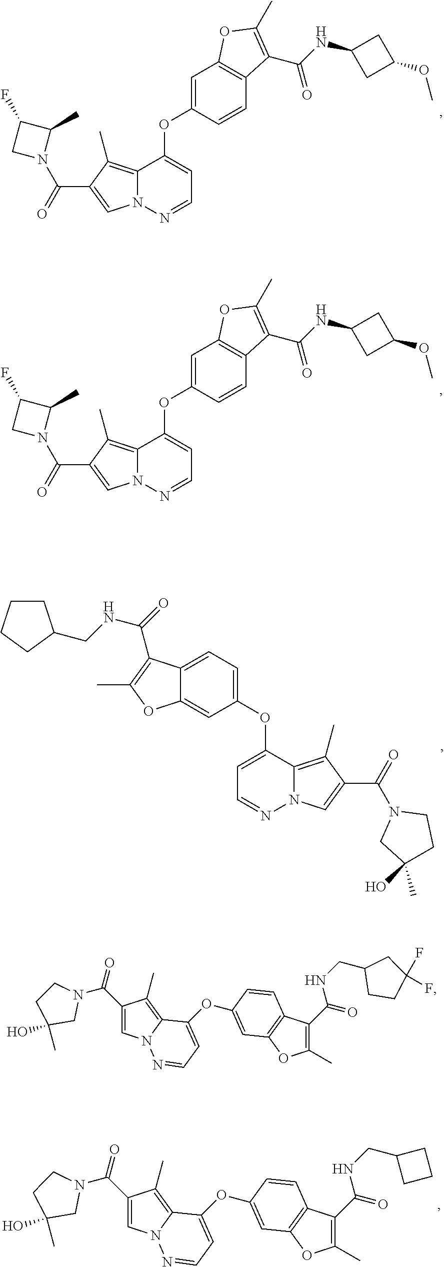 [SMILES: CO[C@H]1C[C@H](NC(=O)c2c(C)oc3cc(Oc4ccnn5cc(C(=O)N6C[C@H](F)[C@H]6C)c(C)c45)ccc23)C1 ; CO[C@H]1C[C@@H](NC(=O)c2c(C)oc3cc(Oc4ccnn5cc(C(=O)N6C[C@H](F)[C@H]6C)c(C)c45)ccc23)C1 ; Cc1oc2cc(Oc3ccnn4cc(C(=O)N5CC[C@@](C)(O)C5)c(C)c34)ccc2c1C(=O)NCC1CCCC1 ; Cc1oc2cc(Oc3ccnn4cc(C(=O)N5CC[C@@](C)(O)C5)c(C)c34)ccc2c1C(=O)NCC1CCC(F)(F)C1 ; Cc1oc2cc(Oc3ccnn4cc(C(=O)N5CC[C@@](C)(O)C5)c(C)c34)ccc2c1C(=O)NCC1CCC1]

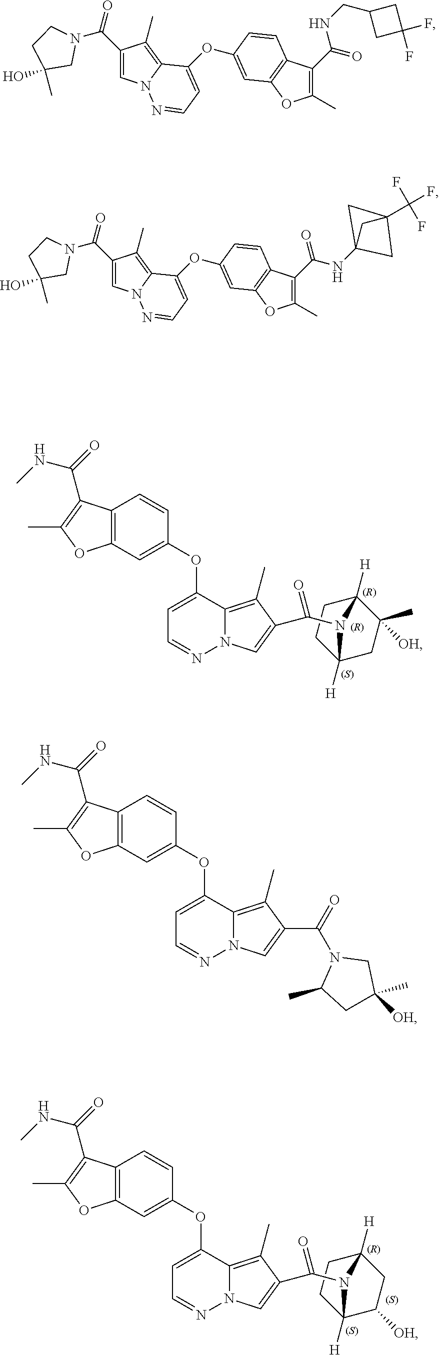

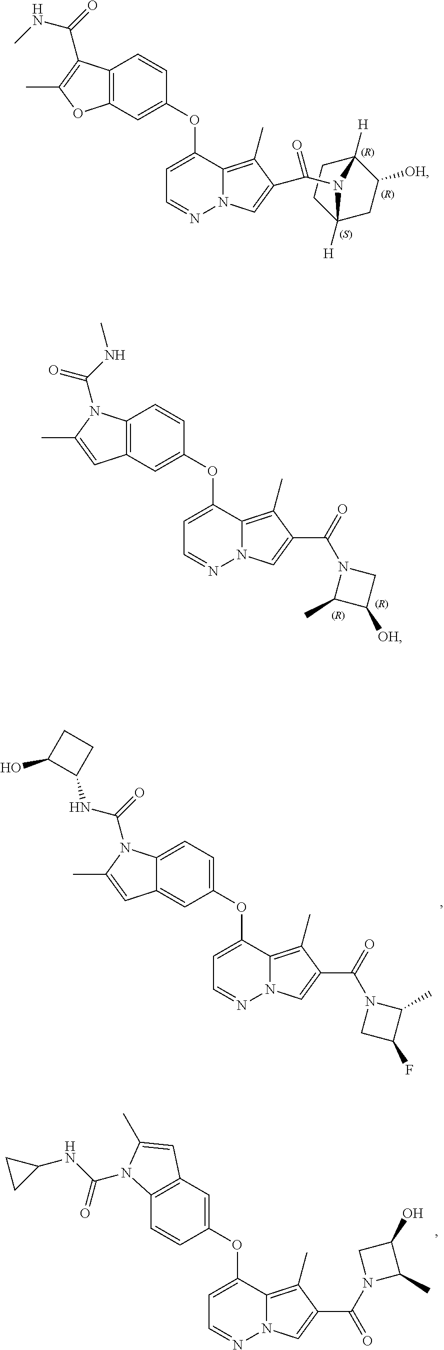

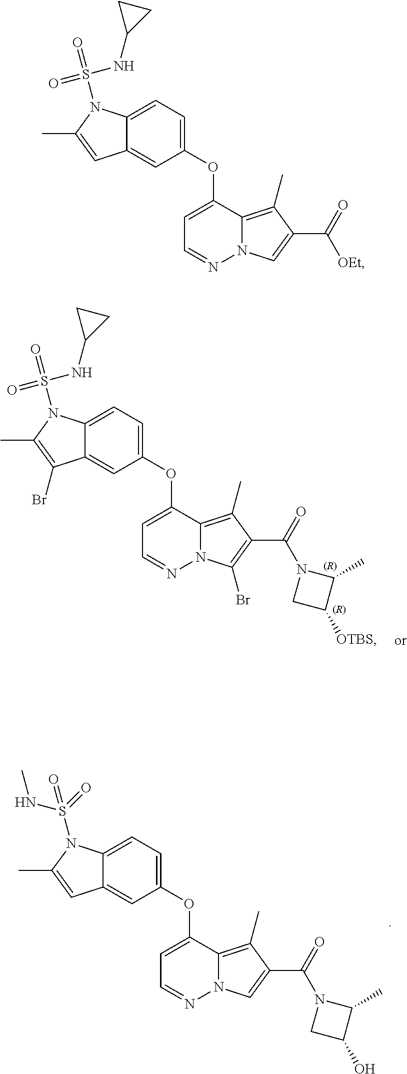

In Embodiment 27 of the disclosure, the compound of formulas I or Ia, or Embodiments 1 to 26 is:
methyl 4-((2-methyl-3-(methylcarbamoyl)benzofuran-6-yl)oxy)pyrrolo[1,2-b]pyridazine-6-carboxylate,
ethyl 4-[(4-fluoro-2-methyl-1H-indol-5-yl)oxy]-5-methylpyrrolo[1,2-b]pyridazine-6-carboxylate,
ethyl 5-methyl-4-{[2-methyl-3-(methylcarbamoyl)-1-benzofuran-6-yl]oxy}pyrrolo[1,2-b]pyridazine-6-carboxylate,
6-((6-((2R,3R)-3-hydroxy-2-methylazetidine-1-carbonyl)-5-methylpyrrolo[1,2-b]pyridazin-4-yl)oxy)-N,2-dimethylbenzofuran-3-carboxamide,
(R)-6-((6-(3-hydroxy-3-methylpyrrolidine-1-carbonyl)-5-methylpyrrolo[1,2-b]pyridazin-4-yl)oxy)-N,2-dimethylbenzofuran-3-carboxamide,
N,2-dimethyl-6-((6-(pyrrolidine-1-carbonyl)pyrrolo[1,2-b]pyridazin-4-yl)oxy)benzofuran-3-carboxamide),
N,2-dimethyl-6-((5-methyl-6-(pyrrolidine-1-carbonyl)pyrrolo[1,2-b]pyridazin-4-yl)oxy)benzofuran-3-carboxamide,
5-methyl-N-(4-methyl-1,1-dioxidotetrahydro-2H-thiopyran-4-yl)-4-((2-methyl-3-(methylcarbamoyl)benzofuran-6-yl)oxy)pyrrolo[1,2-b]pyridazine-6-carboxamide,
6-((6-(4-(2-hydroxypropan-2-yl)-2-azabicyclo[2.1.1]hexane-2-carbonyl)-5-methylpyrrolo[1,2-b]pyridazin-4-yl)oxy)-N,2-dimethylbenzofuran-3-carboxamide,
6-({6-[(3R)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
6-({6-[(3S)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
6-{[6-(3-cyanoazetidine-1-carbonyl)-5-methylpyrrolo[1,2-b]pyridazin-4-yl]oxy}-N,2-dimethyl-1-benzofuran-3-carboxamide,
6-{[6-(3-methoxyazetidine-1-carbonyl)-5-methylpyrrolo[1,2-b]pyridazin-4-yl]oxy}-N,2-dimethyl-1-benzofuran-3-carboxamide,
5-methyl-N-(3-methyl-1,1-dioxo-1lambda˜6˜-thietan-3-yl)-4-{[2-methyl-3-(methylcarbamoyl)-1-benzofuran-6-yl]oxy}pyrrolo[1,2-b]pyridazine-6-carboxamide,
N,2-dimethyl-6-{[5-methyl-6-(7-oxo-2,6-diazaspiro[3.4]octane-2-carbonyl)pyrrolo[1,2-b]pyridazin-4-yl]oxy}-1-benzofuran-3-carboxamide,
6-{[6-(6-hydroxy-2-azaspiro[3.3]heptane-2-carbonyl)-5-methylpyrrolo[1,2-b]pyridazin-4-yl]oxy}-N,2-dimethyl-1-benzofuran-3-carboxamide,
6-({6-[4-(2-hydroxypropan-2-yl)-2-azabicyclo[2.1.1]hexane-2-carbonyl]-5-methylpyrrolo[1,2-b]pyridazin-4-yl}oxy)-N,2-dimethyl-1-benzofuran-3-carboxamide,
(rac)-6-{[6-(4,4-difluorohexahydrocyclopenta[c]pyrrole-2(1H)-carbonyl)-5-methylpyrrolo[1,2-b]pyridazin-4-yl]oxy}-N,2-dimethyl-1-benzofuran-3-carboxamide,
N,2-dimethyl-6-({5-methyl-6-[3-(1H-pyrazol-1-yl)azetidine-1-carbonyl]pyrrolo[1,2-b]pyridazin-4-yl}oxy)-1-benzofuran-3-carboxamide,
N,2-dimethyl-6-{[5-methyl-6-(5-oxa-2-azaspiro[3.4]octane-2-carbonyl)pyrrolo[1,2-b]pyridazin-4-yl]oxy}-1-benzofuran-3-carboxamide,
(rac)-6-{[6-(3-azabicyclo[3.1.0]hexane-3-carbonyl)-5-methylpyrrolo[1,2-b]pyridazin-4-yl]oxy}-N,2-dimethyl-1-benzofuran-3-carboxamide,
N,2-dimethyl-6-({5-methyl-6-[(1S,4S)-2-oxa-5-azabicyclo[2.2.1]heptane-5-carbonyl]pyrrolo[1,2-b]pyridazin-4-yl}oxy)-1-benzofuran-3-carboxamide,
(rac)-N,2-dimethyl-6-{[5-methyl-6-(6-oxo-1,7-diazaspiro[4.4]nonane-1-carbonyl)pyrrolo[1,2-b]pyridazin-4-yl]oxy}-1-benzofuran-3-carboxamide,
(rac)-6-{[6-(hexahydro-4H-furo[3,2-b]pyrrole-4-carbonyl)-5-methylpyrrolo[1,2-b]pyridazin-4-yl]oxy}-N,2-dimethyl-1-benzofuran-3-carboxamide,
(rac)-N,2-dimethyl-6-{[5-methyl-6-(1-oxa-7-azaspiro[4.4]nonane-7-carbonyl)pyrrolo[1,2-b]pyridazin-4-yl]oxy}-1-benzofuran-3-carboxamide,
N,2-dimethyl-6-[(5-methyl-6-{3-[(1H-1,2,4-triazol-3-yl)methyl]azetidine-1-carbonyl}pyrrolo[1,2-b]pyridazin-4-yl)oxy]-1-benzofuran-3-carboxamide,
6-{[6-(3-hydroxy-3-methylazetidine-1-carbonyl)-5-methylpyrrolo[1,2-b]pyridazin-4-yl]oxy}-N,2-dimethyl-1-benzofuran-3-carboxamide,
N,2-dimethyl-6-{[5-methyl-6-(7-oxa-2-azaspiro[3.5]nonane-2-carbonyl)pyrrolo[1,2-b]pyridazin-4-yl]oxy}-1-benzofuran-3-carboxamide,
(rac)-N,2-dimethyl-6-({5-methyl-6-[3-(1H-pyrazol-1-yl)pyrrolidine-1-carbonyl]pyrrolo[1,2-b]pyridazin-4-yl}oxy)-1-benzofuran-3-carboxamide,
N-(3-methoxy-2,2-dimethylpropyl)-5-methyl-4-{[2-methyl-3-(methylcarbamoyl)-1-benzofuran-6-yl]oxy}pyrrolo[1,2-b]pyridazine-6-carboxamide,
(rac)-N-(1-cyclopropylethyl)-5-methyl-4-{[2-methyl-3-(methylcarbamoyl)-1-benzofuran-6-yl]oxy}pyrrolo[1,2-b]pyridazine-6-carboxamide,
N-[(1S,2R)-2-hydroxycyclohexyl]-N,5-dimethyl-4-{[2-methyl-3-(methylcarbamoyl)-1-benzofuran-6-yl]oxy}pyrrolo[1,2-b]pyridazine-6-carboxamide,
(rac)-N,5-dimethyl-4-{[2-methyl-3-(methylcarbamoyl)-1-benzofuran-6-yl]oxy}-N-[(1-methyl-5-oxopyrrolidin-2-yl)methyl]pyrrolo[1,2-b]pyridazine-6-carboxamide,
6-({6-[(2R,4R)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R)-2-(methoxymethyl)pyrrolidine-1-carbonyl]-5-methylpyrrolo[1,2-b]pyridazin-4-yl}oxy)-N,2-dimethyl-1-benzofuran-3-carboxamide,
6-{[6-(2,2-dimethylpyrrolidine-1-carbonyl)-5-methylpyrrolo[1,2-b]pyridazin-4-yl]oxy}-N,2-dimethyl-1-benzofuran-3-carboxamide,
(rac)-6-{[6-(2-azabicyclo[3.1.0]hexane-2-carbonyl)-5-methylpyrrolo[1,2-b]pyridazin-4-yl]oxy}-N,2-dimethyl-1-benzofuran-3-carboxamide,
N,2-dimethyl-6-({5-methyl-6-[(2R)-2-methylpyrrolidine-1-carbonyl]pyrrolo[1,2-b]pyridazin-4-yl}oxy)-1-benzofuran-3-carboxamide,
N,2-dimethyl-6-({5-methyl-6-[(2S)-2-methylpyrrolidine-1-carbonyl]pyrrolo[1,2-b]pyridazin-4-yl}oxy)-1-benzofuran-3-carboxamide,
N,2-dimethyl-6-({5-methyl-6-[(3aS,7aS)-octahydro-1H-indole-1-carbonyl]pyrrolo[1,2-b]pyridazin-4-yl}oxy)-1-benzofuran-3-carboxamide,
(rac)-6-({6-[2-(cyanomethyl)pyrrolidine-1-carbonyl]-5-methylpyrrolo[1,2-b]pyridazin-4-yl}oxy)-N,2-dimethyl-1-benzofuran-3-carboxamide,
6-({6-[(2R,5S)-2,5-dimethylpyrrolidine-1-carbonyl]-5-methylpyrrolo[1,2-b]pyridazin-4-yl}oxy)-N,2-dimethyl-1-benzofuran-3-carboxamide,
(rac)-6-{[6-(3-hydroxy-3-methylpiperidine-1-carbonyl)-5-methylpyrrolo[1,2-b]pyridazin-4-yl]oxy}-N,2-dimethyl-1-benzofuran-3-carboxamide,
(rac)-6-{[6-(3-hydroxypiperidine-1-carbonyl)-5-methylpyrrolo[1,2-b]pyridazin-4-yl]oxy}-N,2-dimethyl-1-benzofuran-3-carboxamide,
(rac)-6-{[6-(2-ethylpiperidine-1-carbonyl)-5-methylpyrrolo[1,2-b]pyridazin-4-yl]oxy}-N,2-dimethyl-1-benzofuran-3-carboxamide,
(trans)-6-{[6-(hexahydro-2H-pyrano[3,2-c]pyridine-6(5H)-carbonyl)-5-methylpyrrolo[1,2-b]pyridazin-4-yl]oxy}-N,2-dimethyl-1-benzofuran-3-carboxamide,
(cis)-6-{[6-(hexahydro-2H-pyrano[3,2-c]pyridine-6(5H)-carbonyl)-5-methylpyrrolo[1,2-b]pyridazin-4-yl]oxy}-N,2-dimethyl-1-benzofuran-3-carboxamide,
(rac)-N,2-dimethyl-6-{[5-methyl-6-(1-oxa-7-azaspiro[4.5]decane-7-carbonyl)pyrrolo[1,2-b]pyridazin-4-yl]oxy}-1-benzofuran-3-carboxamide,
(rac)-N,2-dimethyl-6-{[5-methyl-6-(1-oxo-2-oxa-7-azaspiro[4.5]decane-7-carbonyl)pyrrolo[1,2-b]pyridazin-4-yl]oxy}-1-benzofuran-3-carboxamide,
(rac)-6-{[6-(hexahydrofuro[3,2-b]pyridine-4(2H)-carbonyl)-5-methylpyrrolo[1,2-b]pyridazin-4-yl]oxy}-N,2-dimethyl-1-benzofuran-3-carboxamide,
(rac)-N,2-dimethyl-6-{[5-methyl-6-(2-methyloctahydro-4H-1,4-benzoxazine-4-carbonyl)pyrrolo[1,2-b]pyridazin-4-yl]oxy}-1-benzofuran-3-carboxamide,
N,2-dimethyl-6-({5-methyl-6-[(3S)-3-(propan-2-yl)morpholine-4-carbonyl]pyrrolo[1,2-b]pyridazin-4-yl}oxy)-1-benzofuran-3-carboxamide,
(rac)-6-{[6-(2-azabicyclo[2.2.1]heptane-2-carbonyl)-5-methylpyrrolo[1,2-b]pyridazin-4-yl]oxy}-N,2-dimethyl-1-benzofuran-3-carboxamide,
6-{[6-(azepane-1-carbonyl)-5-methylpyrrolo[1,2-b]pyridazin-4-yl]oxy}-N,2-dimethyl-1-benzofuran-3-carboxamide,
(rac)-6-{[6-(3-hydroxyazepane-1-carbonyl)-5-methylpyrrolo[1,2-b]pyridazin-4-yl]oxy}-N,2-dimethyl-1-benzofuran-3-carboxamide,
N,2-dimethyl-6-{[5-methyl-6-(1,4-oxazepane-4-carbonyl)pyrrolo[1,2-b]pyridazin-4-yl]oxy}-1-benzofuran-3-carboxamide,
(rac)-ethyl 3-(5-methyl-4-{[2-methyl-3-(methylcarbamoyl)-1-benzofuran-6-yl]oxy}pyrrolo[1,2-b]pyridazine-6-carbonyl)-3-azabicyclo[3.1.0]hexane-2-carboxylate,
N,2-dimethyl-6-({5-methyl-6-[(2S)-2-methylmorpholine-4-carbonyl]pyrrolo[1,2-b]pyridazin-4-yl}oxy)-1-benzofuran-3-carboxamide,
N,2-dimethyl-6-({5-methyl-6-[(2R)-2-methylmorpholine-4-carbonyl]pyrrolo[1,2-b]pyridazin-4-yl}oxy)-1-benzofuran-3-carboxamide,
N-[(1R,2R)-2-hydroxycyclohexyl]-N,5-dimethyl-4-{[2-methyl-3-(methylcarbamoyl)-1-benzofuran-6-yl]oxy}pyrrolo[1,2-b]pyridazine-6-carboxamide,
6-({6-[(2S)-2-(methoxymethyl)pyrrolidine-1-carbonyl]-5-methylpyrrolo[1,2-b]pyridazin-4-yl}oxy)-N,2-dimethyl-1-benzofuran-3-carboxamide,
6-({6-[(2S,3R)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R,3R)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R,3S)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2S,4S)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2S,4R)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R,4S)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
N,2-dimethyl-6-({5-methyl-6-[(2S)-2-methylazetidine-1-carbonyl]pyrrolo[1,2-b]pyridazin-4-yl}oxy)-1-benzofuran-3-carboxamide,
N,2-dimethyl-6-({5-methyl-6-[(2R)-2-methylazetidine-1-carbonyl]pyrrolo[1,2-b]pyridazin-4-yl}oxy)-1-benzofuran-3-carboxamide,
6-({6-[(2R)-2-(methoxymethyl)azetidine-1-carbonyl]-5-methylpyrrolo[1,2-b]pyridazin-4-yl}oxy)-N,2-dimethyl-1-benzofuran-3-carboxamide,
6-({6-[(2S)-2-(methoxymethyl)azetidine-1-carbonyl]-5-methylpyrrolo[1,2-b]pyridazin-4-yl}oxy)-N,2-dimethyl-1-benzofuran-3-carboxamide,
6-({6-[(2R,3R)-3-fluoro-2-methylazetidine-1-carbonyl]-5-methylpyrrolo[1,2-b]pyridazin-4-yl}oxy)-N,2-dimethyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3R)-3-fluoro-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3S)-3-fluoro-2-methylazetidine-1-carbonyl]-5-methylpyrrolo[1,2-b]pyridazin-4-yl}oxy)-N,2-dimethyl-1-benzofuran-3-carboxamide,
7-fluoro-6-({6-[(2R,3R)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3S)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3R)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2R,3S)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2R,3S)-3-hydroxy-2-(trifluoromethyl)azetidine-1-carbonyl]-5-methylpyrrolo[1,2-b]pyridazin-4-yl}oxy)-N,2-dimethyl-1-benzofuran-3-carboxamide,
(rac)-6-{[6-(3,3-difluoro-2-methylazetidine-1-carbonyl)-5-methylpyrrolo[1,2-b]pyridazin-4-yl]oxy}-N,2-dimethyl-1-benzofuran-3-carboxamide,
6-({6-[(2S,3S)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-[(6-{(3S)-3-[acetyl(methyl)amino]pyrrolidine-1-carbonyl}-5-methylpyrrolo[1,2-b]pyridazin-4-yl)oxy]-N,2-dimethyl-1-benzofuran-3-carboxamide,
6-[(6-{(3R)-3-[acetyl(methyl)amino]pyrrolidine-1-carbonyl}-5-methylpyrrolo[1,2-b]pyridazin-4-yl)oxy]-N,2-dimethyl-1-benzofuran-3-carboxamide,
(rac)-6-[(6-{3-hydroxy-3-[(1H-1,2,3-triazol-1-yl)methyl]pyrrolidine-1-carbonyl}-5-methylpyrrolo[1,2-b]pyridazin-4-yl)oxy]-N,2-dimethyl-1-benzofuran-3-carboxamide,
(rac)-6-({6-[3-(difluoromethyl)-3-hydroxypyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(pyridin-3-yl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(methoxymethyl)pyrrolidine-1-carbonyl]-5-methylpyrrolo[1,2-b]pyridazin-4-yl}oxy)-N,2-dimethyl-1-benzofuran-3-carboxamide,
(rac)-N,2-dimethyl-6-{[5-methyl-6-(1-oxa-6-azaspiro[3.4]octane-6-carbonyl)pyrrolo[1,2-b]pyridazin-4-yl]oxy}-1-benzofuran-3-carboxamide,
(rac)-6-{[6-(3-ethyl-3-hydroxypyrrolidine-1-carbonyl)-5-methylpyrrolo[1,2-b]pyridazin-4-yl]oxy}-N,2-dimethyl-1-benzofuran-3-carboxamide,
6-{[6-(4-methoxy-2-azabicyclo[2.1.1]hexane-2-carbonyl)-5-methylpyrrolo[1,2-b]pyridazin-4-yl]oxy}-N,2-dimethyl-1-benzofuran-3-carboxamide,
(rac)-6-[(6-{3-hydroxy-3-[4-(trifluoromethyl)phenyl]pyrrolidine-1-carbonyl}-5-methylpyrrolo[1,2-b]pyridazin-4-yl)oxy]-N,2-dimethyl-1-benzofuran-3-carboxamide,
(rac)-6-({6-[3-hydroxy-3-(propan-2-yl)pyrrolidine-1-carbonyl]-5-methylpyrrolo[1,2-b]pyridazin-4-yl}oxy)-N,2-dimethyl-1-benzofuran-3-carboxamide,
6-({6-[(1R,5S)-1-methoxy-3-azabicyclo[3.2.0]heptane-3-carbonyl]-5-methylpyrrolo[1,2-b]pyridazin-4-yl}oxy)-N,2-dimethyl-1-benzofuran-3-carboxamide,
(rac)-6-({6-[3-(cyanomethyl)-3-hydroxypyrrolidine-1-carbonyl]-5-methylpyrrolo[1,2-b]pyridazin-4-yl}oxy)-N,2-dimethyl-1-benzofuran-3-carboxamide,
6-({6-[(1R,5S)-1-hydroxy-3-azabicyclo[3.2.0]heptane-3-carbonyl]-5-methylpyrrolo[1,2-b]pyridazin-4-yl}oxy)-N,2-dimethyl-1-benzofuran-3-carboxamide,
(rac)-6-({6-[3-methoxy-3-(trifluoromethyl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(1-methyl-1H-pyrazol-5-yl)pyrrolidine-1-carbonyl]-5-methylpyrrolo[1,2-b]pyridazin-4-yl}oxy)-N,2-dimethyl-1-benzofuran-3-carboxamide,
(rac)-N,2-dimethyl-6-({5-methyl-6-[1-(2,2,2-trifluoroethoxy)-3-azabicyclo[3.2.0]heptane-3-carbonyl]pyrrolo[1,2-b]pyridazin-4-yl}oxy)-1-benzofuran-3-carboxamide,
N,2-dimethyl-6-{[5-methyl-6-(1′H-spiro[3-oxabicyclo[3.1.0]hexane-2,3′-pyrrolidine]-1′-carbonyl)pyrrolo[1,2-b]pyridazin-4-yl]oxy}-1-benzofuran-3-carboxamide,
(rac)-6-[(6-{3-[(4-chlorophenyl)methyl]-3-hydroxypyrrolidine-1-carbonyl}-5-methylpyrrolo[1,2-b]pyridazin-4-yl)oxy]-N,2-dimethyl-1-benzofuran-3-carboxamide,
(rac)-6-({6-[3-(4-fluorophenyl)-3-hydroxypyrrolidine-1-carbonyl]-5-methylpyrrolo[1,2-b]pyridazin-4-yl}oxy)-N,2-dimethyl-1-benzofuran-3-carboxamide,
(rac)-6-{[6-(3-cyclopropyl-3-hydroxypyrrolidine-1-carbonyl)-5-methylpyrrolo[1,2-b]pyridazin-4-yl]oxy}-N,2-dimethyl-1-benzofuran-3-carboxamide,
(rac)-6-({6-[3-hydroxy-3-(2-methylpropyl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(3-methyl-1,2,4-oxadiazol-5-yl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-methoxy-3-(1-methyl-1H-imidazol-2-yl)pyrrolidine-1-carbonyl]-5-methylpyrrolo[1,2-b]pyridazin-4-yl}oxy)-N,2-dimethyl-1-benzofuran-3-carboxamide,
(rac)-6-{[6-(3-benzyl-3-hydroxypyrrolidine-1-carbonyl)-5-methylpyrrolo[1,2-b]pyridazin-4-yl]oxy}-N,2-dimethyl-1-benzofuran-3-carboxamide,
(rac)-6-({6-[3-hydroxy-3-(trifluoromethyl)pyrrolidine-1-carbonyl]-5-methylpyrrolo[1,2-b]pyridazin-4-yl}oxy)-N,2-dimethyl-1-benzofuran-3-carboxamide,
(rac)-6-{[6-(3,3-dimethyl-1-oxa-6-azaspiro[3.4]octane-6-carbonyl)-5-methylpyrrolo[1,2-b]pyridazin-4-yl]oxy}-N,2-dimethyl-1-benzofuran-3-carboxamide,
(rac)-6-[(6-{3-[(4-fluorophenyl)methyl]-3-hydroxypyrrolidine-1-carbonyl}-5-methylpyrrolo[1,2-b]pyridazin-4-yl)oxy]-N,2-dimethyl-1-benzofuran-3-carboxamide,
(rac)-6-[(6-{3-[(dimethylamino)methyl]-3-hydroxypyrrolidine-1-carbonyl}-5-methylpyrrolo[1,2-b]pyridazin-4-yl)oxy]-N,2-dimethyl-1-benzofuran-3-carboxamide,
6-({6-[(2S,4R)-4-hydroxy-2-(methoxymethyl)pyrrolidine-1-carbonyl]-5-methylpyrrolo[1,2-b]pyridazin-4-yl}oxy)-N,2-dimethyl-1-benzofuran-3-carboxamide,
N-cyclobutyl-6-{[6-(3-methoxyazetidine-1-carbonyl)-5-methylpyrrolo[1,2-b]pyridazin-4-yl]oxy}-2-methyl-1-benzofuran-3-carboxamide,
N-cyclobutyl-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-cyclobutyl-6-({6-[(3S)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-2-methyl-6-({5-methyl-6-[(2R)-2-methylpyrrolidine-1-carbonyl]pyrrolo[1,2-b]pyridazin-4-yl}oxy)-1-benzofuran-3-carboxamide,
N-(bicyclo[1.1.1]pentan-1-yl)-6-({6-[(2R,4R)-4-hydroxy-2-methylpyrrolidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S)-2-(methoxymethyl)pyrrolidine-1-carbonyl]-5-methylpyrrolo[1,2-b]pyridazin-4-yl}oxy)-2-methyl-1-benzofuran-3-carboxamide,
Trans, N-(3,3-difluoro-2-hydroxycyclohexyl)-5-methyl-4-((2-methyl-3-(methylcarbamoyl)benzofuran-6-yl)oxy)pyrrolo[1,2-b]pyridazine-6-carboxamide),
N-(bicyclo[1.1.1]pentan-1-yl)-2-methyl-6-({5-methyl-6-[(2S)-2-methylazetidine-1-carbonyl]pyrrolo[1,2-b]pyridazin-4-yl}oxy)-1-benzofuran-3-carboxamide,
N-(bicyclo[1.1.1]pentan-1-yl)-2-methyl-6-({5-methyl-6-[(2R)-2-methylazetidine-1-carbonyl]pyrrolo[1,2-b]pyridazin-4-yl}oxy)-1-benzofuran-3-carboxamide,
N-(bicyclo[1.1.1]pentan-1-yl)-6-({6-[(2R)-2-(methoxymethyl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S)-2-(methoxymethyl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3R)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3S)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3R)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3S)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3S)-3-hydroxy-2-(trifluoromethyl)azetidine-1-carbonyl]-5-methylpyrrolo[1,2-b]pyridazin-4-yl}oxy)-2-methyl-1-benzofuran-3-carboxamide,
(rac)-N-(bicyclo[1.1.1]pentan-1-yl)-6-{[6-(3,3-difluoro-2-methylazetidine-1-carbonyl)-5-methylpyrrolo[1,2-b]pyridazin-4-yl]oxy}-2-methyl-1-benzofuran-3-carboxamide,
N-(bicyclo[1.1.1]pentan-1-yl)-6-{[6-(6-hydroxy-2-azaspiro[3.3]heptane-2-carbonyl)-5-methylpyrrolo[1,2-b]pyridazin-4-yl]oxy}-2-methyl-1-benzofuran-3-carboxamide,
N-(bicyclo[1.1.1]pentan-1-yl)-6-({6-[3-(difluoromethyl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[3-(2-hydroxypropan-2-yl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-2-methyl-6-{[5-methyl-6-(7-oxo-2,6-diazaspiro[3.4]octane-2-carbonyl)pyrrolo[1,2-b]pyridazin-4-yl]oxy}-1-benzofuran-3-carboxamide,
6-({6-[3-(acetylamino)-3-methylazetidine-1-carbonyl]-5-methylpyrrolo[1,2-b]pyridazin-4-yl}oxy)-N-(bicyclo[1.1.1]pentan-1-yl)-2-methyl-1-benzofuran-3-carboxamide,
N-(bicyclo[1.1.1]pentan-1-yl)-2-methyl-6-{[5-methyl-6-(6-oxo-5-oxa-2,7-diazaspiro[3.4]octane-2-carbonyl)pyrrolo[1,2-b]pyridazin-4-yl]oxy}-1-benzofuran-3-carboxamide,
N-(bicyclo[1.1.1]pentan-1-yl)-2-methyl-6-({5-methyl-6-[3-(2-oxopyrrolidin-1-yl)azetidine-1-carbonyl]pyrrolo[1,2-b]pyridazin-4-yl}oxy)-1-benzofuran-3-carboxamide,
6-({6-[3-(acetylamino)azetidine-1-carbonyl]-5-methylpyrrolo[1,2-b]pyridazin-4-yl}oxy)-N-(bicyclo[1.1.1]pentan-1-yl)-2-methyl-1-benzofuran-3-carboxamide,
N-(bicyclo[1.1.1]pentan-1-yl)-6-{[6-(3-hydroxyazetidine-1-carbonyl)-5-methylpyrrolo[1,2-b]pyridazin-4-yl]oxy}-2-methyl-1-benzofuran-3-carboxamide,
N-(bicyclo[1.1.1]pentan-1-yl)-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-[(1S,2R)-2-fluorocyclobutyl]-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,4S)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,4R)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,3S)-3-hydroxy-2-methylpyrrolidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,3R)-3-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,4S)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,4R)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3S)-3-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3R)-3-hydroxy-2-methylpyrrolidine-1-carbonyl]-5-methylpyrrolo[1,2-b]pyridazin-4-yl}oxy)-2-methyl-1-benzofuran-3-carboxamide,
6-({6-[(3S)-3-hydroxy-3-methylpyrrolidine-1-carbonyl]-5-methylpyrrolo[1,2-b]pyridazin-4-yl}oxy)-N,2,7-trimethyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-N,2,7-trimethyl-1-benzofuran-3-carboxamide,
N-cyclobutyl-6-({6-[(3S)-3-hydroxy-3-methylpyrrolidine-1-carbonyl]-5-methylpyrrolo[1,2-b]pyridazin-4-yl}oxy)-2,7-dimethyl-1-benzofuran-3-carboxamide,
N-cyclobutyl-6-({6-[(3R)-3-hydroxy-3-methylpyrrolidine-1-carbonyl]-5-methylpyrrolo[1,2-b]pyridazin-4-yl}oxy)-2,7-dimethyl-1-benzofuran-3-carboxamide,
7-fluoro-6-({6-[(3S)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
7-fluoro-6-({6-[(3R)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
7-fluoro-6-({6-[(2R,4S)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
(R)-{4-[(4-fluoro-2-methyl-1H-indol-5-yl)oxy]-5-methylpyrrolo[1,2-b]pyridazin-6-yl}(3-hydroxypyrrolidin-1-yl)methanone,
(S)-{4-[(4-fluoro-2-methyl-1H-indol-5-yl)oxy]-5-methylpyrrolo[1,2-b]pyridazin-6-yl}(3-hydroxypyrrolidin-1-yl)methanone,
{4-[(4-fluoro-2-methyl-1H-indol-5-yl)oxy]-5-methylpyrrolo[1,2-b]pyridazin-6-yl}(3-methoxyazetidin-1-yl)methanone,
{4-[(4-fluoro-2-methyl-1H-indol-5-yl)oxy]-5-methylpyrrolo[1,2-b]pyridazin-6-yl}[(3S)-3-methoxypyrrolidin-1-yl]methanone,
{4-[(4-fluoro-2-methyl-1H-indol-5-yl)oxy]-5-methylpyrrolo[1,2-b]pyridazin-6-yl}[(3R)-3-methoxypyrrolidin-1-yl]methanone,
{4-[(4-fluoro-2-methyl-1H-indol-5-yl)oxy]-5-methylpyrrolo[1,2-b]pyridazin-6-yl}[(3S)-3-hydroxy-3-methylpyrrolidin-1-yl]methanone,
{4-[(4-fluoro-2-methyl-1H-indol-5-yl)oxy]-5-methylpyrrolo[1,2-b]pyridazin-6-yl}[(3R)-3-hydroxy-3-methylpyrrolidin-1-yl]methanone,
1-{4-[(4-fluoro-2-methyl-1H-indol-5-yl)oxy]-5-methylpyrrolo[1,2-b]pyridazine-6-carbonyl}azetidine-3-carbonitrile,
6-({6-[(3S)-3-hydroxy-3-methylpyrrolidine-1-carbonyl]-5-methylpyrrolo[1,2-b]pyridazin-4-yl}oxy)-N-methyl-1-benzothiophene-3-carboxamide,
6-({6-[(3R)-3-hydroxy-3-methylpyrrolidine-1-carbonyl]-5-methylpyrrolo[1,2-b]pyridazin-4-yl}oxy)-N-methyl-1-benzothiophene-3-carboxamide,
6-({6-[(3S)-3-hydroxy-3-methylpyrrolidine-1-carbonyl]-5-methylpyrrolo[1,2-b]pyridazin-4-yl}oxy)-N,2-dimethyl-1-benzothiophene-3-carboxamide,
6-({6-[(3R)-3-hydroxy-3-methylpyrrolidine-1-carbonyl]-5-methylpyrrolo[1,2-b]pyridazin-4-yl}oxy)-N,2-dimethyl-1-benzothiophene-3-carboxamide,
6-({6-[(2R,3R)-3-hydroxy-2-methylazetidine-1-carbonyl]-5-methylpyrrolo[1,2-b]pyridazin-4-yl}oxy)-N,2-dimethyl-1-benzothiophene-3-carboxamide,
6-({6-[(2R,4S)-4-hydroxy-2-methylpyrrolidine-1-carbonyl]-5-methylpyrrolo[1,2-b]pyridazin-4-yl}oxy)-N,2-dimethyl-1-benzothiophene-3-carboxamide,
6-({6-[(2R,3R)-3-hydroxy-2-methylpyrrolidine-1-carbonyl]-5-methylpyrrolo[1,2-b]pyridazin-4-yl}oxy)-N,2-dimethyl-1-benzothiophene-3-carboxamide,
N-ethyl-5-({6-[(2R,3R)-3-hydroxy-2-methylazetidine-1-carbonyl]-5-methylpyrrolo[1,2-b]pyridazin-4-yl}oxy)-2-methyl-1H-indole-1-carboxamide,
N-ethyl-5-({6-[(3S)-3-hydroxy-3-methylpyrrolidine-1-carbonyl]-5-methylpyrrolo[1,2-b]pyridazin-4-yl}oxy)-2-methyl-1H-indole-1-carboxamide,
N-ethyl-5-({6-[(3R)-3-hydroxy-3-methylpyrrolidine-1-carbonyl]-5-methylpyrrolo[1,2-b]pyridazin-4-yl}oxy)-2-methyl-1H-indole-1-carboxamide,
N-ethyl-5-((6-(4-hydroxy-2-azabicyclo[2.1.1]hexane-2-carbonyl)-5-methylpyrrolo[1,2-b]pyridazin-4-yl)oxy)-2-methyl-1H-indole-1-carboxamide,
(S)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
(R)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
4-fluoro-5-((6-(4-hydroxy-2-azabicyclo[2.1.1]hexane-2-carbonyl)-5-methylpyrrolo[1,2-b]pyridazin-4-yl)oxy)-N,2-dimethyl-1H-indole-1-carboxamide,
5-((6-((2S,3S)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
5-((6-((2R,3R)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
6-({6-[(2S)-2-(difluoromethyl)azetidine-1-carbonyl]-5-methylpyrrolo[1,2-b]pyridazin-4-yl}oxy)-N,2-dimethyl-1-benzofuran-3-carboxamide,
N,2-dimethyl-6-({5-methyl-6-[(2S)-2-(trifluoromethyl)azetidine-1-carbonyl]pyrrolo[1,2-b]pyridazin-4-yl}oxy)-1-benzofuran-3-carboxamide,
N,2-dimethyl-6-({5-methyl-6-[2-(propan-2-yl)azetidine-1-carbonyl]pyrrolo[1,2-b]pyridazin-4-yl}oxy)-1-benzofuran-3-carboxamide,
6-{[6-(2-cyclopropylazetidine-1-carbonyl)-5-methylpyrrolo[1,2-b]pyridazin-4-yl]oxy}-N,2-dimethyl-1-benzofuran-3-carboxamide,
6-({6-[(2R,4S)-2,4-dimethylazetidine-1-carbonyl]-5-methylpyrrolo[1,2-b]pyridazin-4-yl}oxy)-N,2-dimethyl-1-benzofuran-3-carboxamide,
6-({6-[(2R,4R)-2,4-dimethylazetidine-1-carbonyl]-5-methylpyrrolo[1,2-b]pyridazin-4-yl}oxy)-N,2-dimethyl-1-benzofuran-3-carboxamide,
6-({6-[(2S,4S)-2,4-dimethylazetidine-1-carbonyl]-5-methylpyrrolo[1,2-b]pyridazin-4-yl}oxy)-N,2-dimethyl-1-benzofuran-3-carboxamide,
6-{[6-(6-cyano-2-azaspiro[3.3]heptane-2-carbonyl)-5-methylpyrrolo[1,2-b]pyridazin-4-yl]oxy}-N,2-dimethyl-1-benzofuran-3-carboxamide,
6-{[6-(3,3-dimethylazetidine-1-carbonyl)-5-methylpyrrolo[1,2-b]pyridazin-4-yl]oxy}-N,2-dimethyl-1-benzofuran-3-carboxamide,
(rac)-6-[(6-{3-[(dimethylamino)methyl]-3-hydroxyazetidine-1-carbonyl}-5-methylpyrrolo[1,2-b]pyridazin-4-yl)oxy]-N,2-dimethyl-1-benzofuran-3-carboxamide,
(rac)-6-{[6-(3-ethyl-3-fluoroazetidine-1-carbonyl)-5-methylpyrrolo[1,2-b]pyridazin-4-yl]oxy}-N,2-dimethyl-1-benzofuran-3-carboxamide,
6-{[6-(5-azaspiro[2.3]hexane-5-carbonyl)-5-methylpyrrolo[1,2-b]pyridazin-4-yl]oxy}-N,2-dimethyl-1-benzofuran-3-carboxamide,
(rac)-6-{[6-(3-cyano-3-fluoroazetidine-1-carbonyl)-5-methylpyrrolo[1,2-b]pyridazin-4-yl]oxy}-N,2-dimethyl-1-benzofuran-3-carboxamide,
(rac)-6-{[6-(3-cyano-3-hydroxyazetidine-1-carbonyl)-5-methylpyrrolo[1,2-b]pyridazin-4-yl]oxy}-N,2-dimethyl-1-benzofuran-3-carboxamide,
6-({6-[3-(2-cyanopropan-2-yl)azetidine-1-carbonyl]-5-methylpyrrolo[1,2-b]pyridazin-4-yl}oxy)-N,2-dimethyl-1-benzofuran-3-carboxamide,
(rac)-6-{[6-(3-cyclopropyl-3-methylazetidine-1-carbonyl)-5-methylpyrrolo[1,2-b]pyridazin-4-yl]oxy}-N,2-dimethyl-1-benzofuran-3-carboxamide,
N,2-dimethyl-6-{[5-methyl-6-(1-methyl-2-azabicyclo[2.1.1]hexane-2-carbonyl)pyrrolo[1,2-b]pyridazin-4-yl]oxy}-1-benzofuran-3-carboxamide,
6-({6-[1-(hydroxymethyl)-2-azabicyclo[2.1.1]hexane-2-carbonyl]-5-methylpyrrolo[1,2-b]pyridazin-4-yl}oxy)-N,2-dimethyl-1-benzofuran-3-carboxamide,
6-{[6-(7-azabicyclo[2.2.1]heptane-7-carbonyl)-5-methylpyrrolo[1,2-b]pyridazin-4-yl]oxy}-N,2-dimethyl-1-benzofuran-3-carboxamide,
6-{[6-(6-azabicyclo[3.1.1]heptane-6-carbonyl)-5-methylpyrrolo[1,2-b]pyridazin-4-yl]oxy}-N,2-dimethyl-1-benzofuran-3-carboxamide,
N-(cyclopropylmethyl)-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-{[6-(3-hydroxyazetidine-1-carbonyl)-5-methylpyrrolo[1,2-b]pyridazin-4-yl]oxy}-2-methyl-1-benzofuran-3-carboxamide,
N-(cyclopropylmethyl)-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-(cyclopropylmethyl)-6-({6-[(2R,3S)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,3R)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,3S)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,3R)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,4S)-2,4-dimethylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,4R)-2,4-dimethylazetidine-1-carbonyl]-5-methylpyrrolo[1,2-b]pyridazin-4-yl}oxy)-2-methyl-1-benzofuran-3-carboxamide,
6-{[6-(3-cyano-2-methylazetidine-1-carbonyl)-5-methylpyrrolo[1,2-b]pyridazin-4-yl]oxy}-N-(cyclopropylmethyl)-2-methyl-1-benzofuran-3-carboxamide,
6-({6-[3-(acetylamino)azetidine-1-carbonyl]-5-methylpyrrolo[1,2-b]pyridazin-4-yl}oxy)-N-(cyclopropylmethyl)-2-methyl-1-benzofuran-3-carboxamide,
N-(bicyclo[1.1.1]pentan-1-yl)-6-{[6-(4-hydroxy-2-azabicyclo[2.1.1]hexane-2-carbonyl)-5-methylpyrrolo[1,2-b]pyridazin-4-yl]oxy}-2-methyl-1-benzofuran-3-carboxamide,
7-fluoro-6-{[6-(4-hydroxy-2-azabicyclo[2.1.1]hexane-2-carbonyl)-5-methylpyrrolo[1,2-b]pyridazin-4-yl]oxy}-N,2-dimethyl-1-benzofuran-3-carboxamide,
(R)-N,2-dimethyl-6-((5-methyl-6-(3-(N-methylacetamido)pyrrolidine-1-carbonyl)pyrrolo[1,2-b]pyridazin-4-yl)oxy)benzo[b]thiophene-3-carboxamide,
6-((6-(4-hydroxy-2-azabicyclo[2.1.1]hexane-2-carbonyl)-5-methylpyrrolo[1,2-b]pyridazin-4-yl)oxy)-N,2-dimethylbenzo[b]thiophene-3-carboxamide,
6-({6-[(2S,3S)-3-hydroxy-2-methylazetidine-1-carbonyl]-5-methylpyrrolo[1,2-b]pyridazin-4-yl}oxy)-N,3-dimethyl-1-benzofuran-2-carboxamide,
6-({6-[(2R,3R)-3-hydroxy-2-methylazetidine-1-carbonyl]-5-methylpyrrolo[1,2-b]pyridazin-4-yl}oxy)-N,3-dimethyl-1-benzofuran-2-carboxamide,
6-({6-[(2R,3S)-3-hydroxy-2-methylazetidine-1-carbonyl]-5-methylpyrrolo[1,2-b]pyridazin-4-yl}oxy)-N,3-dimethyl-1-benzofuran-2-carboxamide,
6-{[6-(3-hydroxyazetidine-1-carbonyl)-5-methylpyrrolo[1,2-b]pyridazin-4-yl]oxy}-N,3-dimethyl-1-benzofuran-2-carboxamide,
6-{[6-(3-hydroxy-3-methylazetidine-1-carbonyl)-5-methylpyrrolo[1,2-b]pyridazin-4-yl]oxy}-N,3-dimethyl-1-benzofuran-2-carboxamide,
6-({6-[(2S,3S)-3-hydroxy-2-methylazetidine-1-carbonyl]-5-methylpyrrolo[1,2-b]pyridazin-4-yl}oxy)-N-methyl-1-benzofuran-2-carboxamide,
6-({6-[(2R,3R)-3-hydroxy-2-methylazetidine-1-carbonyl]-5-methylpyrrolo[1,2-b]pyridazin-4-yl}oxy)-N-methyl-1-benzofuran-2-carboxamide,
6-({6-[(2R,3S)-3-hydroxy-2-methylazetidine-1-carbonyl]-5-methylpyrrolo[1,2-b]pyridazin-4-yl}oxy)-N-methyl-1-benzofuran-2-carboxamide,
6-{[6-(3-hydroxyazetidine-1-carbonyl)-5-methylpyrrolo[1,2-b]pyridazin-4-yl]oxy}-N-methyl-1-benzofuran-2-carboxamide,
6-{[6-(3-hydroxy-3-methylazetidine-1-carbonyl)-5-methylpyrrolo[1,2-b]pyridazin-4-yl]oxy}-N-methyl-1-benzofuran-2-carboxamide,
6-({6-[(2R,3S)-3-hydroxy-2-methylazetidine-1-carbonyl]-5-methylpyrrolo[1,2-b]pyridazin-4-yl}oxy)-N,3-dimethyl-1-benzothiophene-2-carboxamide,
6-({6-[(2S,3S)-3-hydroxy-2-methylazetidine-1-carbonyl]-5-methylpyrrolo[1,2-b]pyridazin-4-yl}oxy)-N,3-dimethyl-1-benzothiophene-2-carboxamide,
6-({6-[(2R,3R)-3-hydroxy-2-methylazetidine-1-carbonyl]-5-methylpyrrolo[1,2-b]pyridazin-4-yl}oxy)-N,3-dimethyl-1-benzothiophene-2-carboxamide,
6-{[6-(4-hydroxy-2-azabicyclo[2.1.1]hexane-2-carbonyl)-5-methylpyrrolo[1,2-b]pyridazin-4-yl]oxy}-N,3-dimethyl-1-benzothiophene-2-carboxamide,
6-{[6-(2-azabicyclo[2.1.1]hexane-2-carbonyl)-5-methylpyrrolo[1,2-b]pyridazin-4-yl]oxy}-N,3-dimethyl-1-benzothiophene-2-carboxamide,
6-{[6-(3-hydroxyazetidine-1-carbonyl)-5-methylpyrrolo[1,2-b]pyridazin-4-yl]oxy}-N,3-dimethyl-1-benzothiophene-2-carboxamide,
6-{[6-(3-hydroxy-3-methylazetidine-1-carbonyl)-5-methylpyrrolo[1,2-b]pyridazin-4-yl]oxy}-N,3-dimethyl-1-benzothiophene-2-carboxamide,
6-[(6-{(3S)-3-[acetyl(methyl)amino]pyrrolidine-1-carbonyl}-5-methylpyrrolo[1,2-b]pyridazin-4-yl)oxy]-N,3-dimethyl-1-benzothiophene-2-carboxamide,
6-[(6-{(3R)-3-[acetyl(methyl)amino]pyrrolidine-1-carbonyl}-5-methylpyrrolo[1,2-b]pyridazin-4-yl)oxy]-N,3-dimethyl-1-benzothiophene-2-carboxamide,
6-({6-[(3R)-3-hydroxy-3-methylpyrrolidine-1-carbonyl]-5-methylpyrrolo[1,2-b]pyridazin-4-yl}oxy)-N,3-dimethyl-1-benzothiophene-2-carboxamide,
6-({6-[(2R,3R)-3-hydroxy-2-methylpyrrolidine-1-carbonyl]-5-methylpyrrolo[1,2-b]pyridazin-4-yl}oxy)-N,3-dimethyl-1-benzothiophene-2-carboxamide,
5-({6-[(2R,3R)-3-hydroxy-2-methylazetidine-1-carbonyl]-5-methylpyrrolo[1,2-b]pyridazin-4-yl}oxy)-N,1-dimethyl-1H-benzimidazole-2-carboxamide,
6-((6-((1R,4R)-2-oxa-5-azabicyclo[2.2.1]heptane-5-carbonyl)-5-methylpyrrolo[1,2-b]pyridazin-4-yl)oxy)-7-fluoro-N,2-dimethylbenzofuran-3-carboxamide,
5-((6-(4-hydroxy-2-azabicyclo[2.1.1]hexane-2-carbonyl)-5-methylpyrrolo[1,2-b]pyridazin-4-yl)oxy)-N,2-dimethyl-1H-indole-1-carboxamide,
N-((1R,3R)-3-hydroxycyclobutyl)-N,5-dimethyl-4-((2-methyl-1-(methylcarbamoyl)-1H-indol-5-yl)oxy)pyrrolo[1,2-b]pyridazine-6-carboxamide,
N,2-dimethyl-5-((5-methyl-6-(7-methyl-1,7-diazaspiro[3.5]nonane-1-carbonyl)pyrrolo[1,2-b]pyridazin-4-yl)oxy)-1H-indole-1-carboxamide,
5-((6-(4-(2-hydroxypropan-2-yl)-2-azabicyclo[2.1.1]hexane-2-carbonyl)-5-methylpyrrolo[1,2-b]pyridazin-4-yl)oxy)-N,2-dimethyl-1H-indole-1-carboxamide,
N-ethyl-5-((6-((2R,3R)-3-hydroxy-2,3-dimethylazetidine-1-carbonyl)-5-methylpyrrolo[1,2-b]pyridazin-4-yl)oxy)-2-methyl-1H-indole-1-carboxamide,
N-ethyl-5-((6-((2R,3R)-3-fluoro-2-methylazetidine-1-carbonyl)-5-methylpyrrolo[1,2-b]pyridazin-4-yl)oxy)-2-methyl-1H-indole-1-carboxamide,
5-((6-((1R,5R)-2-oxa-6-azabicyclo[3.2.0]heptane-6-carbonyl)-5-methylpyrrolo[1,2-b]pyridazin-4-yl)oxy)-N-ethyl-2-methyl-1H-indole-1-carboxamide,
5-((6-((1R,5R)-2-oxa-6-azabicyclo[3.2.0]heptane-6-carbonyl)-5-methylpyrrolo[1,2-b]pyridazin-4-yl)oxy)-N,2-dimethyl-1H-indole-1-carboxamide,
(S)-N-ethyl-5-((6-(7-hydroxy-2-oxa-5-azaspiro[3.4]octane-5-carbonyl)-5-methylpyrrolo[1,2-b]pyridazin-4-yl)oxy)-2-methyl-1H-indole-1-carboxamide,
(cis)-N,2-dimethyl-6-((5-methyl-6-(octahydro-1H-indole-1-carbonyl)pyrrolo[1,2-b]pyridazin-4-yl)oxy)benzofuran-3-carboxamide,
(cis)-6-((6-(2,5-dimethylpyrrolidine-1-carbonyl)-5-methylpyrrolo[1,2-b]pyridazin-4-yl)oxy)-N,2-dimethylbenzofuran-3-carboxamide,
(trans)-N-(2-hydroxycyclohexyl)-N,5-dimethyl-4-((2-methyl-3-(methylcarbamoyl)benzofuran-6-yl)oxy)pyrrolo[1,2-b]pyridazine-6-carboxamide,
6-((6-((2R,3R or 2S,3S)-3-cyano-2-methylazetidine-1-carbonyl)-5-methylpyrrolo[1,2-b]pyridazin-4-yl)oxy)-N,2-dimethylbenzofuran-3-carboxamide,
(R)-6-((6-(3-hydroxy-2,2-dimethylazetidine-1-carbonyl)-5-methylpyrrolo[1,2-b]pyridazin-4-yl)oxy)-N,2-dimethylbenzofuran-3-carboxamide,
(S)-6-((6-(3-hydroxy-2,2-dimethylazetidine-1-carbonyl)-5-methylpyrrolo[1,2-b]pyridazin-4-yl)oxy)-N,2-dimethylbenzofuran-3-carboxamide,
6-({6-[(2S,3S or 2R,3R)-3-hydroxy-2,3-dimethylazetidine-1-carbonyl]-5-methylpyrrolo[1,2-b]pyridazin-4-yl}oxy)-N,2-dimethyl-1-benzofuran-3-carboxamide,
6-({6-[(2S,3S)-3-hydroxy-2,3-dimethylazetidine-1-carbonyl]-5-methylpyrrolo[1,2-b]pyridazin-4-yl}oxy)-N,2-dimethyl-1-benzofuran-3-carboxamide,
6-({6-[(2R,3R)-3-hydroxy-2,3-dimethylazetidine-1-carbonyl]-5-methylpyrrolo[1,2-b]pyridazin-4-yl}oxy)-N,2-dimethyl-1-benzofuran-3-carboxamide,
(R)-5-({6-[(2R,3R)-3-hydroxy-2-methylazetidine-1-carbonyl]-5-methylpyrrolo[1,2-b]pyridazin-4-yl}oxy)-N-methyl-2,3-dihydro-1H-indene-1-carboxamide,
(S)-5-({6-[(2R,3R)-3-hydroxy-2-methylazetidine-1-carbonyl]-5-methylpyrrolo[1,2-b]pyridazin-4-yl}oxy)-N-methyl-2,3-dihydro-1H-indene-1-carboxamide,
(S)-N,2-dimethyl-6-((5-methyl-6-(8-oxo-2,7-diazaspiro[4.4]nonane-2-carbonyl)pyrrolo[1,2-b]pyridazin-4-yl)oxy)benzofuran-3-carboxamide,
(R)-N,2-dimethyl-6-((5-methyl-6-(8-oxo-2,7-diazaspiro[4.4]nonane-2-carbonyl)pyrrolo[1,2-b]pyridazin-4-yl)oxy)benzofuran-3-carboxamide,
6-({6-[(2S,3S)-3-methoxy-2-methylazetidine-1-carbonyl]-5-methylpyrrolo[1,2-b]pyridazin-4-yl}oxy)-N,2-dimethyl-1-benzofuran-3-carboxamide,
1-{4-[(2,3-dihydro-1-benzofuran-6-yl)amino]-5-methylpyrrolo[1,2-b]pyridazine-6-carbonyl}azetidine-3-carbonitrile,
N-((1R,3R)-3-(cyanomethyl)cyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
N-((1S,3S)-3-fluorocyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
N-((1R,3R)-3-fluorocyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
N-[(cis)-2-fluorocyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-((1S,3S)-3-(cyanomethyl)cyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[3.3]heptan-2-yl)-1-benzofuran-3-carboxamide,
6-((6-((R)-3-hydroxy-3-methylpyrrolidine-1-carbonyl)-5-methylpyrrolo[1,2-b]pyridazin-4-yl)oxy)-2-methyl-N-((1S,3S)-3-methylcyclobutyl)benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(trans)-2-methylcyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(cis)-2-methylcyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-((S)-spiro[3.3]heptan-1-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-((R)-spiro[3.3]heptan-1-yl)-1-benzofuran-3-carboxamide,
N-({R}2,2-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-({S}2,2-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-((6-((R)-3-hydroxy-3-methylpyrrolidine-1-carbonyl)-5-methylpyrrolo[1,2-b]pyridazin-4-yl)oxy)-2-methyl-N-((1S,3S)-1-oxidothietan-3-yl)benzofuran-3-carboxamide,
6-((6-((R)-3-hydroxy-3-methylpyrrolidine-1-carbonyl)-5-methylpyrrolo[1,2-b]pyridazin-4-yl)oxy)-2-methyl-N-((1R,3R)-1-oxidothietan-3-yl)benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-(oxetan-3-yl)-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-(3-methyloxetan-3-yl)-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-[(3R)-oxolan-3-yl]-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-[(3S)-3-methyloxolan-3-yl]-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(3-hydroxybicyclo[11.1.1]pentan-1-yl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-1[(1S,2R)-2-hydroxycyclobutyl]-2-methyl-1-benzofuran-3-carboxamide 6-({6-[(2R,3S)-3-fluoro-2-methylazetidine-1-carbonyl]-5-methylpyrrolo[1,2-b]pyridazin-4-yl}oxy)-N-[(1R,2R)-2-hydroxycyclobutyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-[(1S,2S)-2-hydroxycyclobutyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-[(1R,2S)-2-hydroxycyclobutyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(trans-3-hydroxycyclobutyl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(cis-3-hydroxycyclobutyl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-[(2R)-2-hydroxypropyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-(1-methylcyclobutyl)-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(trans-3-methoxycyclobutyl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(cis-3-methoxycyclobutyl)-2-methyl-1-benzofuran-3-carboxamide,
N-[((S)-3,3-difluorocyclopentyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[((R)-3,3-difluorocyclopentyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-((6-((1R,2R,4S)-2-hydroxy-2-methyl-7-azabicyclo[2.2.1]heptane-7-carbonyl)-5-methylpyrrolo[1,2-b]pyridazin-4-yl)oxy)-N,2-dimethylbenzofuran-3-carboxamide,
6-((6-((2R,4S or 2R,4R)-4-hydroxy-2,4-dimethylpyrrolidine-1-carbonyl)-5-methylpyrrolo[1,2-b]pyridazin-4-yl)oxy)-N,2-dimethylbenzofuran-3-carboxamide,
6-((6-((1S,2S,4R)-2-hydroxy-7-azabicyclo[2.2.1]heptane-7-carbonyl)-5-methylpyrrolo[1,2-b]pyridazin-4-yl)oxy)-N,2-dimethylbenzofuran-3-carboxamide,
6-((6-((1R,2R,4S)-2-hydroxy-7-azabicyclo[2.2.1]heptane-7-carbonyl)-5-methylpyrrolo[1,2-b]pyridazin-4-yl)oxy)-N,2-dimethylbenzofuran-3-carboxamide,
5-((6-((2R,3S)-3-fluoro-2-methylazetidine-1-carbonyl)-5-methylpyrrolo[1,2-b]pyridazin-4-yl)oxy)-N-((1S,2S)-2-hydroxycyclobutyl)-2-methyl-1H-indole-1-carboxamide,
N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-carboxamide,
5-({6-[(2R,3R)-3-hydroxy-2-methylazetidine-1-carbonyl]-5-methylpyrrolo[1,2-b]pyridazin-4-yl}oxy)-N,2-dimethyl-1H-indole-1-sulfonamide,
7-fluoro-6-({6-[(2R,3R)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
N-[(1R,2S)-2-fluorocyclobutyl]-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-{[6-(3-hydroxyazetidine-1-carbonyl)-5-methylpyrrolo[1,2-b]pyridazin-4-yl]oxy}-2-methyl-1-benzofuran-3-carboxamide,
N-[(1R,2S)-2-fluorocyclobutyl]-6-{[6-(3-hydroxyazetidine-1-carbonyl)-5-methylpyrrolo[1,2-b]pyridazin-4-yl]oxy}-2-methyl-1-benzofuran-3-carboxamide,
N-[(1S,2R)-2-fluorocyclobutyl]-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-[(1R,2S)-2-fluorocyclobutyl]-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-[(1R,2S)-2-fluorocyclobutyl]-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
Cis,6-((6-(3-amino-2-methylazetidine-1-carbonyl)-5-methylpyrrolo[1,2-b]pyridazin-4-yl)oxy)-N,2-dimethylbenzofuran-3-carboxamide,
Trans,N-(3,3-difluoro-2-hydroxycyclohexyl)-5-methyl-4-((2-methyl-3-(methylcarbamoyl)benzofuran-6-yl)oxy)pyrrolo[1,2-b]pyridazine-6-carboxamide),
(N,2-dimethyl-6-((5-methyl-6-(8-oxo-2,7-diazaspiro[4.4]nonane-2-carbonyl)pyrrolo[1,2-b]pyridazin-4-yl)oxy)benzofuran-3-carboxamide),
(R)-6-{[6-(3,3-difluoro-4-hydroxypiperidine-1-carbonyl)-5-methylpyrrolo[1,2-b]pyridazin-4-yl]oxy}-N,2-dimethyl-1-benzofuran-3-carboxamide,
(S)-6-{[6-(3,3-difluoro-4-hydroxypiperidine-1-carbonyl)-5-methylpyrrolo[1,2-b]pyridazin-4-yl]oxy}-N,2-dimethyl-1-benzofuran-3-carboxamide,
(R)-6-{[6-(3-methoxy-3-methylpyrrolidine-1-carbonyl)-5-methylpyrrolo[1,2-b]pyridazin-4-yl]oxy}-N,2-dimethyl-1-benzofuran-3-carboxamide,
(S)-6-{[6-(3-methoxy-3-methylpyrrolidine-1-carbonyl)-5-methylpyrrolo[1,2-b]pyridazin-4-yl]oxy}-N,2-dimethyl-1-benzofuran-3-carboxamide,
1-(4-((1H-indol-5-yl)oxy)-5-methylpyrrolo[1,2-b]pyridazine-6-carbonyl)azetidine-3-carbonitrile,
6-((6-(3-cyanoazetidine-1-carbonyl)-5-methylpyrrolo[1,2-b]pyridazin-4-yl)oxy)-N-cyclopropyl-2-methylbenzofuran-3-carboxamide,
6-{[6-(3-cyanoazetidine-1-carbonyl)-5-methylpyrrolo[1,2-b]pyridazin-4-yl]oxy}-N-cyclobutyl-2-methyl-1-benzofuran-3-carboxamide,
1-{4-[(2,3-dimethyl-1-benzofuran-6-yl)oxy]-5-methylpyrrolo[1,2-b]pyridazine-6-carbonyl}azetidine-3-carbonitrile,
6-{[6-(3-cyanoazetidine-1-carbonyl)-5-methylpyrrolo[1,2-b]pyridazin-4-yl]oxy}-N,2-dimethyl-1-benzothiophene-3-carboxamide,
1-{5-methyl-4-[(2-methyl-1H-indol-5-yl)oxy]pyrrolo[1,2-b]pyridazine-6-carbonyl}azetidine-3-carbonitrile,
1-{4-[(1H-indol-5-yl)oxy]-5-methylpyrrolo[1,2-b]pyridazine-6-carbonyl}azetidine-3-carbonitrile,
1-{4-[(1-benzofuran-7-yl)oxy]-5-methylpyrrolo[1,2-b]pyridazine-6-carbonyl}azetidine-3-carbonitrile,
1-{4-[(1-benzothiophen-5-yl)oxy]-5-methylpyrrolo[1,2-b]pyridazine-6-carbonyl}azetidine-3-carbonitrile,
1-{4-[(1-benzothiophen-6-yl)oxy]-5-methylpyrrolo[1,2-b]pyridazine-6-carbonyl}azetidine-3-carbonitrile,
ethyl (5-{[6-(3-cyanoazetidine-1-carbonyl)-5-methylpyrrolo[1,2-b]pyridazin-4-yl]oxy}-2,3-dihydro-1H-inden-2-yl)acetate,
1-{5-methyl-4-[(3-methyl-1-benzofuran-6-yl)oxy]pyrrolo[1,2-b]pyridazine-6-carbonyl}azetidine-3-carbonitrile,
1-{4-[(1-benzofuran-6-yl)oxy]-5-methylpyrrolo[1,2-b]pyridazine-6-carbonyl}azetidine-3-carbonitrile,
1-{5-methyl-4-[(3-methyl-1-benzothiophen-6-yl)oxy]pyrrolo[1,2-b]pyridazine-6-carbonyl}azetidine-3-carbonitrile,
1-{4-[(1H-indol-6-yl)oxy]-5-methylpyrrolo[1,2-b]pyridazine-6-carbonyl}azetidine-3-carbonitrile,
1-{4-[(2,3-dihydro-1-benzofuran-6-yl)oxy]-5-methylpyrrolo[1,2-b]pyridazine-6-carbonyl}azetidine-3-carbonitrile,
1-{4-[(1-benzofuran-5-yl)oxy]-5-methylpyrrolo[1,2-b]pyridazine-6-carbonyl}azetidine-3-carbonitrile,
methyl 6-{[6-(3-cyanoazetidine-1-carbonyl)-5-methylpyrrolo[1,2-b]pyridazin-4-yl]oxy}-2,3-dihydro-1-benzofuran-2-carboxylate,
N-benzyl-5-{[6-(3-cyanoazetidine-1-carbonyl)-5-methylpyrrolo[1,2-b]pyridazin-4-yl]oxy}-2-methyl-1-benzofuran-3-carboxamide,
1-{4-[(2,3-dimethyl-1H-indol-5-yl)oxy]-5-methylpyrrolo[1,2-b]pyridazine-6-carbonyl}azetidine-3-carbonitrile,
ethyl 5-{[6-(3-cyanoazetidine-1-carbonyl)-5-methylpyrrolo[1,2-b]pyridazin-4-yl]oxy}-2-methyl-1-benzofuran-3-carboxylate,
1-{4-[(2,3-dihydro-1-benzothiophen-6-yl)oxy]-5-methylpyrrolo[1,2-b]pyridazine-6-carbonyl}azetidine-3-carbonitrile,
methyl 7-{[6-(3-cyanoazetidine-1-carbonyl)-5-methylpyrrolo[1,2-b]pyridazin-4-yl]oxy}-1-benzofuran-4-carboxylate,
5-{[6-(3-cyanoazetidine-1-carbonyl)-5-methylpyrrolo[1,2-b]pyridazin-4-yl]oxy}-N,2-dimethyl-1-benzofuran-3-carboxamide,
methyl 6-{[6-(3-cyanoazetidine-1-carbonyl)-5-methylpyrrolo[1,2-b]pyridazin-4-yl]oxy}-1H-indole-3-carboxylate,
[(3R)-3-hydroxy-3-methylpyrrolidin-1-yl]{5-methyl-4-[(2-methyl-1H-indol-6-yl)oxy]pyrrolo[1,2-b]pyridazin-6-yl}methanone,
N-ethyl-5-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-ethyl-5-((6-(4-hydroxy-2-azabicyclo[2.1.1]hexane-2-carbonyl)-5-methylpyrrolo[1,2-b]pyridazin-4-yl)oxy)-2-methyl-1H-indole-1-carboxamide,
(S)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
(R)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
4-fluoro-5-((6-(4-hydroxy-2-azabicyclo[2.1.1]hexane-2-carbonyl)-5-methylpyrrolo[1,2-b]pyridazin-4-yl)oxy)-N,2-dimethyl-1H-indole-1-carboxamide,
5-((6-((2S,3S)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
5-((6-((2R,3R)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
1-{5-methyl-4-[(2-methyl-1-benzofuran-5-yl)amino]pyrrolo[1,2-b]pyridazine-6-carbonyl}azetidine-3-carbonitrile,
1-{4-[(1-benzothiophen-6-yl)amino]-5-methylpyrrolo[1,2-b]pyridazine-6-carbonyl}azetidine-3-carbonitrile,
1-{4-[(1-benzothiophen-5-yl)amino]-5-methylpyrrolo[1,2-b]pyridazine-6-carbonyl}azetidine-3-carbonitrile,
1-{4-[(2,3-dihydro-1-benzofuran-6-yl)amino]-5-methylpyrrolo[1,2-b]pyridazine-6-carbonyl}azetidine-3-carbonitrile,
1-{4-[(1-benzofuran-4-yl)amino]-5-methylpyrrolo[1,2-b]pyridazine-6-carbonyl}azetidine-3-carbonitrile,
N-benzyl-6-{[6-(3-cyanoazetidine-1-carbonyl)-5-methylpyrrolo[1,2-b]pyridazin-4-yl]oxy}-2-methyl-1-benzofuran-3-carboxamide,
(R)-N-(cyclopentylmethyl)-6-((6-(3-hydroxy-3-methylpyrrolidine-1-carbonyl)-5-methylpyrrolo[1,2-b]pyridazin-4-yl)oxy)-2-methylbenzofuran-3-carboxamide,
6-{[6-(3-cyanoazetidine-1-carbonyl)-5-methylpyrrolo[1,2-b]pyridazin-4-yl]oxy}-N-(cyclopropylmethyl)-2-methyl-1-benzofuran-3-carboxamide,
N-(bicyclo[1.1.1]pentan-1-yl)-6-{[6-(3-cyanoazetidine-1-carbonyl)-5-methylpyrrolo[1,2-b]pyridazin-4-yl]oxy}-2-methyl-1-benzofuran-3-carboxamide,
1-(4-{[3-(3-fluoroazetidine-1-carbonyl)-2-methyl-1-benzofuran-6-yl]oxy}-5-methylpyrrolo[1,2-b]pyridazine-6-carbonyl)azetidine-3-carbonitrile,
N-benzyl-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[trans-3-(cyanomethyl)cyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(3-cyanocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(cis-3-fluorocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[cis-3-(cyanomethyl)cyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[3.3]heptan-2-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(cis-3-methylcyclobutyl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(1R,2R)-2-methylcyclobutyl]-1-benzofuran-3-carboxamide,
N-(trans-3-fluorocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[3-(propan-2-yl)cyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[3.3]heptan-1-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[3-(trifluoromethyl)cyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[2.3]hexan-5-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(1-oxaspiro[3.3]heptan-6-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(trans-3-methylcyclobutyl)-1-benzofuran-3-carboxamide,
N-(3,3-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(2,2-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(3,3-difluorocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(1-oxo-1lambda˜4˜-thietan-3-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(1S,2R)-2-methylcyclobutyl]-1-benzofuran-3-carboxamide,
N-(1-fluorospiro[2.3]hexan-5-yl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(1-oxo-1lambda˜4˜-thietan-3-yl)-1-benzofuran-3-carboxamide,
N-[(3,3-difluorocyclopentyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(cyclobutylmethyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[(3,3-difluorocyclobutyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-((6-((1S,2S,4R)-2-hydroxy-2-methyl-7-azabicyclo[2.2.1]heptane-7-carbonyl)-5-methylpyrrolo[1,2-b]pyridazin-4-yl)oxy)-N,2-dimethylbenzofuran-3-carboxamide,
5-((6-((2R,3R)-3-hydroxy-2-methylazetidine-1-carbonyl)-5-methylpyrrolo[1,2-b]pyridazin-4-yl)oxy)-N,2-dimethyl-1H-indole-1-carboxamide,
ethyl 4-((1-(N-cyclopropylsulfamoyl)-2-methyl-1H-indol-5-yl)oxy)-5-methylpyrrolo[1,2-b]pyridazine-6-carboxylate,
N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-sulfonamide,
6-({6-[(3R)-3-hydroxy-3-methylpyrrolidine-1-carbonyl]-5-methylpyrrolo[1,2-b]pyridazin-4-yl}oxy)-2-methyl-N-[3-(trifluoromethyl)bicyclo[1.1.1]pentan-1-yl]-1-benzofuran-3-carboxamide, or
N-(3-fluorobicyclo[1.1.1]pentan-1-yl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide, or a pharmaceutically acceptable salt of any of the foregoing.

The present disclosure includes the pharmaceutically acceptable salts of the compounds defined therein.

In one embodiment, the present disclosure is a composition comprising an effective amount of at least one compound of Formula I, or a pharmaceutically acceptable salt thereof.

Also provided is a pharmaceutical composition comprising an effective amount of at least one compound of Formula I, or a pharmaceutically acceptable salt thereof, and a pharmaceutically acceptable carrier.

Further provided is a pharmaceutical composition comprising an effective amount of at least one compound of Formula I, or a pharmaceutically acceptable salt thereof, and an effective amount of at least one other pharmaceutically active ingredient (such as, for example, a chemotherapeutic agent).

In one embodiment, the present disclosure provides a method for treating cancer in a patient, comprising administering to the patient a compound of Formula I, or a pharmaceutically acceptable salt thereof, or a pharmaceutical composition comprising a compound of Formula I. In a further embodiment, the cancer is associated with VEGFR upregulation.

In one embodiment, the present disclosure provides a method for inhibiting the progress of cancer in a patient, comprising administering to the patient a compound of Formula I, or a pharmaceutically acceptable salt thereof, or a pharmaceutical composition comprising a compound of Formula I.

In one embodiment, the present disclosure provides a method of treating a disease or disorder in which VEGFR inhibition is beneficial, comprising administering to a patient in need thereof a therapeutically effective amount of a compound of Formula I, or a pharmaceutically acceptable salt thereof, or a pharmaceutical composition comprising a compound of Formula I. In a further embodiment, the disease or disorder is a cellular proliferative disorder. In a further embodiment, the cellular proliferative disorder is cancer.

In one embodiment, the present disclosure provides for the use of a compound of Formula I, or a pharmaceutically acceptable salt thereof, or a pharmaceutical composition containing a compound of Formula I, for treating cancer in a patient. In one embodiment, the present disclosure provides for the use of a compound of Formula I, or a pharmaceutically acceptable salt thereof, or a pharmaceutical composition containing a compound of Formula I, for the preparation of a medicament useful for the prevention of a cell proliferative disorder.

In one embodiment, the present disclosure provides for the use of a compound of Formula I, or a pharmaceutically acceptable salt thereof, or a pharmaceutical composition containing a compound of Formula I, for treating tumor angiogenesis and lymphangiogenesis.

The methods of the disclosure include the administration of a pharmaceutical composition comprising at least one compound of Formula I, or a pharmaceutically acceptable salt thereof, and a pharmaceutically acceptable carrier.

“Alkyl” means branched- and straight-chain saturated aliphatic hydrocarbon groups having the specified number of carbon atoms when noted. If no number is specified, 1-6 carbon atoms are intended for linear and 3-7 carbon atoms for branched alkyl groups. Examples of alkyl groups include methyl, ethyl, propyl, isopropyl, cyclopropyl, butyl, sec- and tert-butyl, pentyl, hexyl, octyl, nonyl, and the like. For example, the term “C₁₋₆alkyl” includes all of “C₁₋₄alkyl” defined as follows, plus the linear or branched chain alkyl groups, including all possible isomers, having 5 or 6 carbon atoms. “C₁₋₆alkyl” means linear or branched chain alkyl groups, including all possible isomers, having 1, 2, 3, 4, 5 or 6 carbon atoms, and includes each of the alkyl groups within C₁₋₆alkyl including each of the hexyl and pentyl isomers as well as n-, iso-, sec- and tert-butyl (butyl, i-butyl, s-butyl, t-butyl, collectively “C₄alkyl”; Bu=butyl), n- and i-propyl (propyl, i-propyl, collectively “C₃alkyl”; Pr=propyl), ethyl (Et) and methyl (Me). Commonly used abbreviations for alkyl groups are used throughout the specification, e.g., methyl may be represented by conventional abbreviations including “Me” or CH₃ or a symbol that is an extended bond as the terminal group, e.g.,

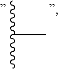

ethyl may be represented by “Et” or CH₂CH₃, propyl may be represented by “Pr” or CH₂CH₂CH₃, butyl may be represented by “Bu” or CH₂CH₂CH₂CH₃, etc. For example, the structures

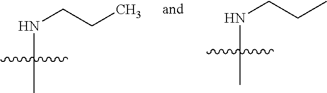

have equivalent meanings. C₁₋₆ alkyl includes n-, iso, sec- and t-butyl, n- and isopropyl, ethyl and methyl. If no number is specified, 1-6 carbon atoms are intended for linear or branched alkyl groups.

“Cyclic amine” refers to a cyclic ring comprising one nitrogen atom. “Alkoxy” refers to an alkyl group linked to oxygen. Examples of alkoxy groups include methoxy, ethoxy, propoxy and the like.

“Aryl” refers to a monocyclic, bicyclic or tricyclic carbocyclic aromatic ring or ring system containing 5-14 carbon atoms, wherein at least one of the rings is aromatic. Examples of aryl include phenyl and naphthyl.

“Fused Aryl” refers to an aryl ring fused with heterocyclyl or cycloalkyl.

“Halogen” or “Halo” includes fluorine, chlorine, bromine and iodine.

“Cycloalkyl” refers to a saturated monocyclic, bicyclic or bridged carbocyclic ring, having a specified number of carbon atoms. Examples of cycloalkyl include cyclopropyl, cyclobutyl, cyclopentyl, cyclohexyl, bicyclopentanyl. The term “cycloalkyl” also includes a C₃₋₆cycloalkyl group fused to a benzene ring such as dihydro-indenyl, tetrahydronaphthalenyl,

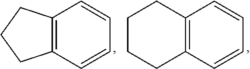

and the like.

“Haloalkyl” refers to an alkyl group as defined within, wherein one or more of the alkyl group's hydrogen atoms has been replaced with a halogen. In one embodiment, a haloalkyl group has from 1 to 6 carbon atoms. Non-limiting examples of haloalkyl groups include CH₂F, CHF₂, CF₃, CH₂CF₃, CH₂CHF₂, CF₂CF₃, CF₂CHF₂, CH₂C₁ and CCl₃. The term “C₁₋₆haloalkyl” or “haloC₁₋₆alkyl” refers to a haloalkyl group having from 1 to 6 carbons.

“Haloalkoxy,” “haloalkyl-O” and derivatives such as “halo(C₁₋₆)alkoxy” are used interchangeably and refer to halo substituted alkyl groups linked through the oxygen atom. Haloalkoxy include mono-substituted as well as multiple halo substituted alkoxy groups. For example, trifluoromethoxy, chloromethoxy, and bromomethoxy are included as well as OCH₂CF₃, OCH₂CHF₂, OCF₂CF₃, and OCF₂CHF₂.

“Heteroaryl”, as used herein, refers to an aromatic monocyclic or multicyclic ring system comprising about 5 to about 14 ring atoms, wherein from 1 to 4 of the ring atoms is independently O, N, SO₂, or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyridazinyl, pyridopyridinyl, pyridyl, pyrimidinyl, pyrrolyl, pyridinyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxybenzene, benzothiazolyl, benzothienyl, quinolinyl, isoquinolinyl, oxazolyl, tetra-hydroquinoline, quinolinyl, pyrrolo[1,2-b]pyridazinyl, benzo[d]thiazolyl, benzo[d]isothiazolyl, pyrrolo[2,3-b]pyridinyl, and benzo[c][1,2,5]thiadiazolyl.

“Heterocyclyl,” or “heterocyclic” refers to “Heterocyclyl” means a saturated or partly unsaturated non-aromatic monocyclic, bicyclic, spirocyclic or bridged ring or ring system having a specified number of carbon atoms and containing at least one ring heteroatom selected from N, NH, S (including SO and SO₂) and O. The cycloheteroalkyl ring may be substituted on the ring carbons and/or the ring nitrogen or sulfur. Examples of cycloheteroalkyl include tetrahydrofuranyl, pyrrolidinyl, tetrahydrothiophenyl, azetidinyl, piperazinyl, piperidinyl, morpholinyl, oxetanyl and tetrahydropyranyl. In one embodiment, cycloheteroalkyl is selected from: pyrrolidinyl, azetidinyl, piperidinyl, piperazinyl, azepanyl, azocanyl, morpholinyl, thiomorpholinyl, thiomorpholine dionyl, oxazepanyl, 1,4-thiazepanyl, isoindolinyl, dihydroisoquinolinyl, tetra-hydroisoquinolinyl, octahydro-isoindolyl, azabicyclo[2.2.1]heptanyl, oxa-azabicyclo[2.2.1]-yl, azabicyclo[3.1.1]heptanyl, azabicyclo[4.1.0]heptanyl, azabicyclo[3.2.1]octanyl, diazaspiro[3.4]octanyl, azaspiro[3.3]heptanyl, azabicyclo[2.1.1]hexanyl, azaspiro[2.3]hexanyl, hexahydrocyclopenta[c]pyrrolyl, azabicyclo[3.1.0]hexanyl, oxa-azabicyclo[2.2.1]heptanyl, diazaspiro[4.4]nonanyl, oxa-azaspiro[4.4]nonanyl, oxa-azaspiro[3.4]octanyl, oxa-azaspiro[bicyclo[3.1.0]hexane-pyrrolidinyl], hexahydropyrano[3,2-c]pyridinyl, hexahydrofuro[3,2-b]pyridinyl, oxadiazaspiro[3.4]octanyl, azabicyclo[2.2.1]heptanyl, diazabicyclo[3.2.1]octanyl, oxa-azabicyclo[3.2.1]octanyl, azabicyclo[3.2.0]heptanyl, oxa-azabicyclo[3.2.0]heptanyl, azaspiro[2.5]octanyl, azaspiro[2.6]nonanyl, azaspiro[3.5]nonanyl, oxa-azaspiro[3.5]nonanyl, oxaazabicyclo[3.2.0]heptanyl, oxa-azaspiro[4.5]decanyl, octahydro-indolyl, dihydrothieno[3,2-c]pyridinyl, dihydro-thiazolo[4,5-c]pyridinyl, dihydrooxazolo[4,5-c]pyridiyle, dihydroimidazo[1,2-a]pyrazinyl, hexahydrofuro[3,2-b]pyrrolyl, octahydrocyclpenta[c]pyrrolyl, dihydro-quinolinyl, dihydro-benzo[b][1,4]oxazinyl, octahydrobenzo[b][1,4]oxazinyl, dihydrobenzo[b][1,4]dioxinyl, dihydrobenzofuranyl, dihydro-pyrrolo[2,3-b]pyridinyl, and dihydrobenzo[b]thiophenyl.

“Bicyclic heterocyclyl,” “bicyclic heterocycle” or “bicyclic heterocyclic” refers to a heterocyclic ring fused to another ring system. The fusion may be bridged or unbridged.

“Fused heteroaryl” is heteroaryl fused with an aryl or heteroaryl.

“Fused heterocyclyl” is a heterocyclyl fused with another heterocyclyl.

“Oxo” means an oxygen linked to an atom by a double bond. An example of an oxo group is a doubly bonded oxygen in a ketone, sulfoxide, sulfone and sulfate.

“Hydroxyalkyl” or “-hydroxy(C₁₋₃)alkyl” means an alkyl group having one or more hydrogen atoms replaced by hydroxyl (—OH) groups

“Cyanoalkyl” means an alkyl group having one or more hydrogen atoms replaced by cyano (—CN) groups.

“Hydroxyhaloalkyl” means an alkyl group having one or more hydrogen atoms replaced by hydroxyl (—OH) groups, and one or more hydrogen atoms replaced by a halogen.

“Hydroxycycloalkyl” means a cyclic alkyl group having one or more hydrogen atoms replaced by hydroxyl (—OH) groups.

The term “composition” is intended to encompass a product comprising the specified ingredients in the specified amounts, as well as any product which results, directly or indirectly, from combination of the specified ingredients in the specified amounts.

The term “at least one” means one or more than one. The meaning of “at least one” with reference to the number of compounds of the present disclosure is independent of the meaning with reference to the number of chemotherapeutic agents.

The term “chemotherapeutic agent” means a drug (medicament or pharmaceutically active ingredient) for treating cancer (i.e., an antineoplastic agent).

The term “effective amount” means a “therapeutically effective amount”. The term “therapeutically effective amount” means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, and also refers to an effect that results in the inhibition of growth and/or metastasis of the cancer.

Except where noted herein, the term “carbocycle” (and variations thereof such as “carbocyclic” or “carbocyclyl”) as used herein, unless otherwise indicated, refers to a C₃ to C₆ monocyclic ring, e.g., C₃₋₆ monocyclic carbocycle. The carbocycle may be attached to the rest of the molecule at any carbon atom which results in a stable compound. Saturated carbocyclic rings include, for example, “cycloalkyl” rings, e.g., cyclopropyl, cyclobutyl, etc. Unsaturated carbocyclic rings include, for example

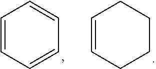

Except where noted, the term “saturated heterocycle” refers to a stable 4- to 7-membered mono-cyclic heteroatom-containing ring system, and which consists of carbon atoms and from one to four heteroatoms independently selected from the group consisting of N, O and S, and wherein the nitrogen and sulfur heteroatoms may optionally be oxidized, and the nitrogen heteroatom may optionally be quaternized. Especially useful are rings containing one oxygen or sulfur, one to four nitrogen atoms, or one oxygen or sulfur combined with one or two nitrogen atoms. The heterocyclic ring may be attached at any heteroatom or carbon atom which results in the creation of a stable structure. Representative examples include azetidine, oxetane, thietane, diazetidine, dioxetane, dithietane, pyrrolidine, tetrahydrofuran, thiolane, imidazolidine, pyrazolidine, oxazolidine, isoxazolidine, thiazolidine, isothiazolidine, dioxolane, dithiolane, piperidine, oxane, thiane, piperazine, morpholine, thiomorpholine, dioxane, dithiane, trioxane, trithiane, azepane, oxepane, thiepane and homopiperazine. For example, a “saturated 6 membered heterocycle” is a stable 6-membered mono-cyclic heteroatom-containing ring system. An example of a saturated 6 membered heterocycle is piperidine. Likewise, but not limiting, a “saturated 4 membered heterocycle” is a stable 4-membered mono-cyclic heteroatom-containing ring system.

Except where noted herein, the term “unsaturated heterocycle” refers to a monocyclic unsaturated heterocycle having a specified number of ring atom members (e.g., 5 membered), including a specified number of heteroatoms (e.g., 1, 2, 3 or 4 heteroatoms independently selected from N, O or S), e.g., 5-membered rings containing one nitrogen (pyrrole), one oxygen (furan) or one sulfur (thiophene) atom, 5-membered rings containing one nitrogen and one sulfur (thiazole) atom, 5-membered rings containing one nitrogen and one oxygen (oxazole or isoxazole) atom, 5-membered rings containing two nitrogen (imidazole or pyrazole) atoms, five-membered aromatic rings containing three nitrogen (triazole) atoms, five-membered aromatic rings containing one oxygen, one nitrogen or one sulfur atom, five-membered aromatic rings containing two heteroatoms independently selected from oxygen, nitrogen and sulfur (e.g., oxazole), Additional examples are thiophene, imidazole, isothiazole, oxadiazole, and isoxazole. For example, a “unsaturated 6 membered heterocycle” is a 6 membered ring containing 6 atom members including at least one heteroatom. Likewise, an “unsaturated 5 membered heterocycle” is a 5 membered ring containing 5 atom members including at least one heteroatom.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The compounds of the present disclosure are limited to stable compounds embraced by Formula I and its embodiments. For example, certain moieties as defined in Formula I, may be unsubstituted or substituted, and the latter is intended to encompass substitution patterns (i.e., number and kind of substituents) that are chemically possible for the moiety and that result in a stable compound.

The term “substituted” means that one or more hydrogens on the designated atom is replaced with a selected from the indicated group, provided that the designated atom's normal valency under the existing circumstances is not exceeded, and that the substitution results in a stable compound. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different. Combinations of substituents and/or variables are permissible only if such combinations result in stable compounds. If a substituent is itself substituted with more than one group, it is understood that these multiple groups may be on the same carbon or on different carbons, so long as a stable structure result. By optionally substituted, it is meant that compounds containing the specified optional substituent(s) as well as compounds that do not contain the optional substituent(s).

The wavy line [symbol] as used herein, indicates a point of attachment to the rest of the compound.

Where ring atoms are represented by variables such as “X”, e.g.,

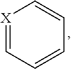

the variables are defined by indicating the atom located at the variable ring position without depicting the ring bonds associated with the atom. For example, when X in the above ring is nitrogen, the definition will show “N” and will not depict the bonds associated with it, e.g., will not show “═N—”. Likewise, when X is a carbon atom that is substituted with bromide, the definition will show “C—Br” and will not depict the bonds associated with it, e.g., will not show

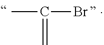

The disclosure also includes derivatives of the compound of formula I, acting as prodrugs and solvates. Any pharmaceutically acceptable pro-drug modification of a compound of Formula I which results in conversion in vivo to a compound within the scope of the disclosure is also contemplated by the present disclosure. Prodrugs, following administration to the patient, are converted in the body by normal metabolic or chemical processes, such as through hydrolysis in the blood, to the compound of Formula I. Such prodrugs include those that demonstrate enhanced bioavailability, tissue specificity, and/or cellular delivery, to improve drug absorption of the compound of I. The effect of such prodrugs may result from modification of physicochemical properties such as lipophilicity, molecular weight, charge, and other physicochemical properties that determine the permeation properties of the drug.

For example, esters can optionally be made by esterification of an available carboxylic acid group or by formation of an ester on an available hydroxy group in a compound. Similarly, labile amides can be made. Pharmaceutically acceptable esters or amides of the compounds of the Formula I may be prepared to act as pro-drugs which can be hydrolyzed back to an acid (or —COO⁻ depending on the pH of the fluid or tissue where conversion takes place) or hydroxy form particularly in vivo and as such are encompassed within the scope of the disclosure. Included are those esters and acyl groups known in the art for modifying the solubility or hydrolysis characteristics for use as sustained-release or prodrug formulations. Examples of pharmaceutically acceptable pro-drug modifications include, but are not limited to, —C₁₋₆alkyl esters and —C₁₋₆alkyl substituted with phenyl esters.

“Celite®” (Fluka) diatomite is diatomaceous earth, and can be referred to as “celite”.

When any variable (e.g., R¹ etc.) occurs more than one time in any constituent or in Formula I or other generic Formula herein, its definition on each occurrence is independent of its definition at every other occurrence. Combinations of substituents and/or variables are permissible only if such combinations result in stable compounds. In choosing compounds of Formula I, one of ordinary skill in the art will recognize that the various substituents, i.e., i.e., R¹ etc., are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heterocyclic ring) provided such ring substitution is chemically allowed and results in a stable compound.

It should be noted that, if a discrepancy between the chemical name and structure exists, the structure is understood to dominate.

Compounds of structural Formula I may contain one or more asymmetric centers and can thus occur as racemates and racemic mixtures, single enantiomers, diastereoisomeric mixtures and individual diastereoisomers. Centers of asymmetry that are present in the compounds of Formulas I can all independently of one another have S configuration or R configuration. When bonds to the chiral carbon are depicted as straight lines in the structural Formulas, it is understood that both the (R) and (S) configurations of the chiral carbon, and hence both enantiomers and mixtures thereof, are embraced within the Formulas. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of the present disclosure.

The compounds of this disclosure include all possible enantiomers and diastereomers and mixtures of two or more stereoisomers, for example mixtures of enantiomers and/or diastereomers, in all ratios. Thus, enantiomers are a subject of the present disclosure in enantiomerically pure form, both as levorotatory and as dextrorotatory antipodes, in the form of racemates and in the form of mixtures of the two enantiomers in all ratios. In the case of a cis/trans isomerism, both the cis form and the trans form as well as mixtures of these forms in all ratios are included unless specified otherwise. The present disclosure is meant to comprehend all such stereo-isomeric forms of the compounds of structural Formula I.

Compounds of structural Formula I may be separated into their individual diastereoisomers by, for example, fractional crystallization from a suitable solvent, for example MeOH or EtOAc or a mixture thereof, or via chiral chromatography using an optically active stationary phase. Optionally a derivatization can be carried out before a separation of stereoisomers. The separation of a mixture of stereoisomers can be carried out at an intermediate step during the synthesis of a compound of Formula I, or it can be done on a final racemic product. Absolute stereochemistry may be determined by X-ray crystallography of crystalline products or crystalline intermediates which are derivatized, if necessary, with a reagent containing an asymmetric center of known absolute configuration. Alternatively, any stereoisomer or isomers of a compound of Formula I may be obtained by stereospecific synthesis using optically pure starting materials or reagents of known absolute configuration. The present disclosure includes all such isomers, as well as salts, solvates (including hydrates) and solvated salts of such racemates, enantiomers, diastereomers and tautomers and mixtures thereof.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

For compounds of Formula I described herein which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of Formula I.

In the compounds of structural Formula I, the atoms may exhibit their natural isotopic abundances, or one or more of the atoms may be artificially enriched in a particular isotope having the same atomic number, but an atomic mass or mass number different from the atomic mass or mass number predominately found in nature. As described and claimed herein is meant to include all suitable isotopic variations of the compounds of structural Formula I and embodiments thereof are intended to be included. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within structural Formula I, can be prepared without undue experimentation by conventional techniques well known to those skilled in the art or by processes analogous to those described in the Schemes and Examples herein using appropriate isotopically-enriched reagents and/or intermediates.

It will be understood that the compounds of structural Formula I may be prepared as pharmaceutically acceptable salts or as salts that are not pharmaceutically acceptable when they are used as precursors to the free compounds or their pharmaceutically acceptable salts or in other synthetic manipulations. The compounds of the present disclosure, including the compounds of the Examples, may also include all salts of the compounds of Formula I which, owing to low physiological compatibility, are not directly suitable for use in pharmaceuticals but which can be used, for example, as intermediates for chemical reactions or for the preparation of physiologically acceptable salts.

The compounds of the present disclosure may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids.

Salts of basic compounds encompassed within the term “pharmaceutically acceptable salt” refer to non-toxic salts of the compounds of this disclosure which are generally prepared by reacting the free base with a suitable organic or inorganic acid. Representative salts of basic compounds of the present disclosure include, but are not limited to, the following: acetate, ascorbate, benzenesulfonate, benzoate, bicarbonate, bisulfate, bitartrate, borate, bromide, butyrate, camphorate, camphorsulfonate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, methanesulfonate, mucate, napsylate, nitrate, N-methylglucamine ammonium salt, oleate, oxalate, pamoate (embonate), palmitate, pantothenate, phosphate/diphosphate, polygalacturonate, propionate, salicylate, stearate, sulfate, subacetate, succinate, tannate, tartrate, teoclate, thiocyanate, tosylate, triethiodide, valerate and the like. Furthermore, where the compounds of the disclosure carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. In one embodiment, the salts of acidic compounds are as follows, the ammonium, calcium, magnesium, potassium, and sodium salts.

With basic reagents such as hydroxides, carbonates, hydrogencarbonates, alkoxides and ammonia, organic bases or alternatively basic amino acids the compounds of the formula I, form stable alkali metal, alkaline earth metal or optionally substituted ammonium salts.

Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also, included are the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The preparation of pharmacologically acceptable salts from compounds of the formula I, capable of salt formation, including their stereoisomeric forms is carried out known methods, for example, by mixing a compound of Formula I with an equivalent amount and a solution containing a desired acid, base, or the like, and then collecting the desired salt by filtering the salt or distilling off the solvent. The compounds of the present disclosure and salts thereof may form solvates with a solvent such as water, ethanol, or glycerol. The compounds of the present invention may form an acid addition salt and a salt with a base at the same time according to the type of substituent of the side chain.

If the compounds of Formula I simultaneously contain acidic and basic groups in the molecule the disclosure also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts.

The present disclosure includes compounds of structural Formula I, as well as salts thereof, particularly pharmaceutically acceptable salts, solvates of such compounds and solvated salt forms thereof, where such forms are possible unless specified otherwise.

Furthermore, compounds of the present disclosure may exist in amorphous form and/or one or more crystalline forms, and as such all amorphous and crystalline forms and mixtures thereof of the compounds of Formula I, including the Examples, are intended to be included within the scope of the present disclosure. In addition, some of the compounds of the instant disclosure may form solvates with water (i.e., a hydrate) or common organic solvents such as but not limited to EtOAc. Such solvates and hydrates, particularly the pharmaceutically acceptable solvates and hydrates, of the instant compounds are likewise encompassed within the scope of this disclosure, along with un-solvated and anhydrous forms.

Accordingly, the compounds within the generic structural formulas, embodiments and specific compounds described in the Examples and claimed herein encompass salts, all possible stereoisomers and tautomers, physical forms (e.g., amorphous and crystalline forms), solvate and hydrate forms thereof and any combination of these forms, as well as the salts, pro-drug forms thereof, and salts of pro-drug forms thereof, where such forms are possible unless specified otherwise,

Further provided are medicaments containing at least one compound of the Formula I and/or of a pharmaceutically acceptable salt of the compound of the Formula I and/or an optionally stereoisomeric form of the compound of the Formulas I or a pharmaceutically acceptable salt of the stereoisomeric form of the compound of Formulas I, together with a pharmaceutically acceptable vehicle, carrier, additive and/or other active substances and auxiliaries.

The medicaments according to the present disclosure can be administered by oral, inhalative, rectal or transdermal administration or by subcutaneous, intraarticular, intraperitoneal or intravenous injection. Oral administration is preferred. Coating of stents with compounds of the Formula I and other surfaces which come into contact with blood in the body is possible.

Also provided is a process for the production of a medicament, which comprises bringing at least one compound of the Formula I into a suitable administration form using a pharmaceutically acceptable carrier and optionally further suitable active substances, additives or auxiliaries.

The present disclosure also relates to processes for the preparation of the compounds of Formula I which are described in the following and by which the compounds of Formula I are obtainable.

The terms “therapeutically effective (or efficacious) amount” and similar descriptions such as “an amount efficacious for treatment” are intended to mean that amount of a pharmaceutical drug that will alleviate the symptoms of the disorder, condition or disease being treated (i.e., disorder, condition or disease associated with VEGFR activity) in an animal or human. The terms “prophylactically effective (or efficacious) amount” and similar descriptions such as “an amount efficacious for prevention” are intended to mean that amount of a pharmaceutical drug that will prevent or reduce the symptoms or occurrence of the disorder, condition or disease being treated (i.e., disorder, condition or disease associated with VEGFR activity) in an animal or human. The dosage regimen utilizing a compound of Formula I is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of cancer.

Disorders, conditions and diseases which can be treated or prevented by inhibiting VEGFR by using the compounds of Formula I or are, for example, cancer, and related diseases and conditions.

The compounds of Formula I and their pharmaceutically acceptable salts can be administered to animals, preferably to mammals, and in particular to humans, as pharmaceuticals by themselves, in mixtures with one another or in the form of pharmaceutical preparations. The term “patient” includes animals, preferably mammals and especially humans, who use the instant active agents for the prevention or treatment of a medical condition. Administering of the drug to the patient includes both self-administration and administration to the patient by another person.

The patient may need, or desire, treatment for an existing disease or medical condition, or may be in need of or desire prophylactic treatment to prevent or reduce the risk of occurrence of said disease or medical condition. As used herein, a patient “in need” of treatment of an existing condition or of prophylactic treatment encompasses both a determination of need by a medical professional as well as the desire of a patient for such treatment.

Furthermore, pharmaceutical preparations (or pharmaceutical compositions) which comprise as active component a therapeutically effective dose of at least one compound of Formula I and/or a pharmaceutically acceptable salt thereof and a customary pharmaceutically acceptable carrier, i.e., one or more pharmaceutically acceptable carrier substances and/or additives.

Thus, an aspect of this disclosure is, for example, said compound of Formula I and its pharmaceutically acceptable salts for use as a pharmaceutical, pharmaceutical preparations which comprise as active component a therapeutically effective dose of said compound and/or a pharmaceutically acceptable salt thereof and a customary pharmaceutically acceptable carrier, and the uses of said compound and/or a pharmaceutically acceptable salt thereof in the therapy or prophylaxis of the above mentioned syndromes as well as their use for preparing medicaments for these purposes.

The pharmaceuticals according to the disclosure can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods. The preferred administration form depends, for example, on the disease to be treated and on its severity.

For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiologically sodium chloride solution, alcohols such as ethanol, glycerol, polyols, sucrose, invert sugar, glucose, mannitol, vegetable oils, etc. It is also possible to lyophilize the compounds of Formula I and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used.

Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

Besides the active compounds and carriers, the pharmaceutical preparations can also contain customary additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The dosage of the active compound of Formula I and/or of a pharmaceutically acceptable salt thereof to be administered depends on the individual case and is, as is customary, to be adapted to the individual circumstances to achieve an optimum effect. Thus, it depends on the nature and the severity of the disorder, condition or disease to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to compounds of Formula I.

### Combination Agents

The compounds of Formula I and their pharmaceutically acceptable salts, hydrates, and solvates can be administered alone or in combination with one or more additional therapeutic agents disclosed herein or other suitable agents, depending on the condition being treated. Hence, in some embodiments the one or more compounds of Formula I or a pharmaceutically acceptable salt, hydrate or ester thereof, will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula I and any of the agents described above can be formulated together in the same dosage form and administered simultaneously. Alternatively, a compound of Formula I and any of the agents described above can be simultaneously administered, wherein both the agents are present in separate formulations. In another alternative, a compound of Formula I can be administered just followed by and any of the agents described above, or vice versa. In some embodiments of the separate administration protocol, a compound of Formula I and any of the agents described above are administered a few minutes apart, or a few hours apart, or a few days apart.

Further contemplated is the treatment of the disease/conditions with a combination of pharmaceutically active compounds that may be administered separately, separate pharmaceutical compositions may be provided in kit form. The kit comprises two separate pharmaceutical compositions: a compound of Formula I, and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral, parenteral; IV, transdermal and subcutaneous), are administered at different dosage intervals, or when titration of the individual components of the combination is desired by the prescribing health care professional.

One or more additional pharmacologically active agents may be administered in combination with a compound of Formula I. An additional active agent (or agents) is intended to mean a pharmaceutically active agent (or agents) that is active in the body, including pro-drugs that convert to pharmaceutically active form after administration, which are different from the compound of Formula I, and also includes free-acid, free-base and pharmaceutically acceptable salts of said additional active agents. Generally, any suitable additional active agent or agents, including but not limited to anti-hypertensive agents, anti-obetic, anti-inflammatory, anti-fibrotic, and anti-atherosclerotic agents such as a lipid modifying compound, anti-diabetic agents and/or anti-obesity agents may be used in any combination with the compound of Formula I in a single dosage formulation (a fixed dose drug combination), or may be administered to the patient in one or more separate dosage formulations which allows for concurrent or sequential administration of the active agents (co-administration of the separate active agents).

Examples of additional active agents which may be employed include but are not limited to angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan i.e., COZAAR®, valsartan, candesartan, olmesartan, telmesartan and any of these drugs used in combination with hydrochlorothiazide such as HYZAAR®); neutral endopeptidase inhibitors (e.g., thiorphan and phosphoramidon), aldosterone antagonists, aldosterone synthase inhibitors, renin inhibitors (e.g., urea derivatives of di- and tri-peptides, amino acids and derivatives, amino acid chains linked by non-peptidic bonds, di- and tri-peptide derivatives, peptidyl amino diols and peptidyl beta-aminoacyl aminodiol carbamates; also, and small molecule renin inhibitors including diol sulfonamides and, N-morpholino derivatives, N-heterocyclic alcohols and pyrolimidazolones; also, pepstatin derivatives and fluoro- and chloro-derivatives of statone-containing peptides, enalkrein, RO 42-5892, A 65317, CP 80794, ES 1005, ES 8891, SQ 34017, aliskiren (2(S),4(S),5(S),7(S)-N-(2-carbamoyl-2-methylpropyl)-5-amino-4-hydroxy-2,7-diisopropyl-8-[4-methoxy-3-(3-methoxypropoxy)-phenyl]-octanamid hemifumarate) SPP600, SPP630 and SPP635), endothelin receptor antagonists, phosphodiesterase-5 inhibitors (e.g., sildenafil, tadalfil and vardenafil), vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazosin, prazosin or alpha methyldopa) central alpha adrenergic agonists, peripheral vasodilators (e.g., hydralazine); lipid lowering agents e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), fluvastatin (particularly the sodium salt sold in LESCOL®), cerivastatin, and pitavastatin; a cholesterol absorption inhibitor such as ezetimibe (ZETIA®) and ezetimibe in combination with any other lipid lowering agents such as the HMG-CoA reductase inhibitors noted above and particularly with simvastatin (VYTORIN®) or with atorvastatin calcium; niacin in immediate-release or controlled release forms, and/or with an HMG-CoA reductase inhibitor; niacin receptor agonists such as acipimox and acifran, as well as niacin receptor partial agonists; anti-cholesterol agents such as PCSK9 inhibitors (alirocumab, evolocumab), Nexletol™ (bempedoic acid, ACL inhibitor), and Vascepa® (Icosapent ethyl); metabolic altering agents including insulin and insulin mimetics (e.g., insulin degludec, insulin glargine, insulin lispro), dipeptidyl peptidase-IV (DPP-4) inhibitors (e.g., sitagliptin, alogliptin, omarigliptin, linagliptin, vildagliptin); insulin sensitizers, including (i) β-klotho/FGFR1 activating monoclonal antibody (e.g., MK-3655), pan FGFR1-4/KLB modulators, FGF19 analogue (e.g., Aldafermin) (ii) PPARγ agonists, such as the glitazones (e.g., pioglitazone, AMG 131, CHS 131, MBX2044, mitoglitazone, lobeglitazone, IDR-105, rosiglitazone, and balaglitazone), and other PPAR ligands, including (1) PPARα/γ dual agonists (e.g. ZYH2, ZYH1, GFT505, chiglitazar, muraglitazar, aleglitazar, sodelglitazar, and naveglitazar); (2) PPARa agonists such as fenofibric acid derivatives (e.g., gemfibrozil, clofibrate, ciprofibrate, fenofibrate, bezafibrate), (3) selective PPARγ modulators (SPPARyM's), (e.g., such as those disclosed in WO 02/060388, WO 02/08188, WO 2004/019869, WO 2004/020409, WO 2004/020408, and WO 2004/066963); (4) PPARγ partial agonists, (5) PPAR a/6 dual agonists (e.g., Elafibranor); (iii) biguanides, such as metformin and its pharmaceutically acceptable salts, in particular, metformin hydrochloride, and extended-release formulations thereof, such as Glumetza™, Fortamet™, and GlucophageXR™; and (iv) protein tyrosine phosphatase-1B (PTP-1B) inhibitors (e.g., ISIS-113715 and TTP814); insulin or insulin analogs (e.g., insulin detemir, insulin glulisine, insulin degludec, insulin glargine, insulin lispro and inhalable formulations of each); leptin and leptin derivatives and agonists; amylin and amylin analogs (e.g., pramlintide); sulfonylurea and non-sulfonylurea insulin secretagogues (e.g., tolbutamide, glyburide, glipizide, glimepiride, mitiglinide, meglitinides, nateglinide and repaglinide); α-glucosidase inhibitors (e.g., acarbose, voglibose and miglitol); glucagon receptor antagonists (e.g., MK-3577, MK-0893, LY-2409021 and KT6-971); incretin mimetics, such as GLP-1, GLP-1 analogs, derivatives, and mimetics; and GLP-1 receptor agonists (e.g., dulaglutide, semaglutide, albiglutide, exenatide, liraglutide, lixisenatide, taspoglutide, CJC-1131, and BIM-51077, including intranasal, transdermal, and once-weekly formulations thereof), bile acid sequestering agents (e.g., colestilan, colestimide, colesevalam hydrochloride, colestipol, cholestyramine, and dialkylaminoalkyl derivatives of a cross-linked dextran), acyl CoA: cholesterol acyltransferase inhibitors, (e.g., avasimibe); antiobesity compounds; agents intended for use in inflammatory conditions, such as aspirin, non-steroidal anti-inflammatory drugs or NSAIDs, glucocorticoids, and selective cyclooxygenase-2 or COX-2 inhibitors; glucokinase activators (GKAs) (e.g., AZD6370); inhibitors of 11β-hydroxysteroid dehydrogenase type 1, (e.g., such as those disclosed in U.S. Pat. No. 6,730,690, and LY-2523199); CETP inhibitors (e.g., anacetrapib, torcetrapib, and evacetrapib); inhibitors of fructose 1,6-bisphosphatase, (e.g., such as those disclosed in U.S. Pat. Nos. 6,054,587; 6,110,903; 6,284,748; 6,399,782; and 6,489,476); inhibitors of acetyl CoA carboxylase-1 or 2 (ACC1 or ACC2); AMP-activated Protein Kinase (AMPK) activators; other agonists of the G-protein-coupled receptors: (i) GPR-109, (ii) GPR-119 (e.g., MBX2982 and PSN821), and (iii) GPR-40 (e.g., TAK875); SSTR3 antagonists (e.g., such as those disclosed in WO 2009/001836); neuromedin U receptor agonists (e.g., such as those disclosed in WO 2009/042053, including, but not limited to, neuromedin S (NMS)); SCD modulators (e.g., Aramchol); GPR-105 antagonists (e.g., such as those disclosed in WO 2009/000087); SGLT inhibitors (e.g., ASP1941, SGLT-3, SGLT-2 such as empagliflozin, dapagliflozin, canagliflozin, and ertugliflozin, BI-10773, remogloflozin, TS-071, tofogliflozin, ipragliflozin, and LX-4211); inhibitors of acyl coenzyme A carboxylase (ACC, MK-4074); inhibitors of diacylglycerol acyltransferase 1 and 2 (DGAT-1 and DGAT-2); inhibitors of fatty acid synthase; inhibitors of acyl coenzyme A: monoacylglycerol acyltransferase 1 and 2 (MGAT-1 and MGAT-2); agonists of the TGR5 receptor (also known as GPBAR1, BG37, GPCR19, GPR131, and M-BAR); ileal bile acid transporter inhibitors; bile acid modulators; PACAP, PACAP mimetics, and PACAP receptor 3 agonists; IL-1b antibodies, (e.g., XOMA052 and canakinumab); anti-fibrotic and/or anti-inflammatory agents (CCR2/CCR5 dual receptor antagonist (e.g., cenicriviroc); galectin 3 inhibitor (e.g., belapectin, GB-1107, GB-1211), siRNA against HSP 47 (e.g., BMS-986263); NSAID derived from pirfenidone (e.g., hydronidone), A3AR agonist (e.g., namodenoson, FM101); TGFTX4 (e.g., nitazoxanide); 5-lipoxygenase inhibitor (e.g., tipelukast), Bifunctional urate inhibitor (e.g., ACQT1127), adiponectin receptor agonist (e.g., ALY688), TNF receptor antagonist (e.g., atrosimab), Autotaxin inhibitor (e.g., BLD-0409, TJC 0265, TJC 0316), CCL24 blocking monoclonal antibody (e.g., CM101), IL-11 inhibitor (e.g., ENx 108A), LPA1 receptor antagonist (e.g., EPGN 696), Dual JAK1/2 inhibitor (e.g., EX 76545), GPR antagonist (e.g., GPR91 antagonist), Integrin avo1, avP3 and avP6 inhibitor (e.g., IDL 2965), NLRP3 antagonist (e.g., IFM-514), inflammasome inhibitors (e.g., JT194, JT349), Cell membrane permeability inhibitor (e.g., Larazotide), CCR5 antagonist (e.g., Ieronlimab), TNF inhibitor (e.g., LIVNate), integrin αvβ6 inhibitor (e.g., MORF beta6), NLRP inflammasome antagonists, siRNA (e.g., OLX 701), dual TFGβ/Hedgehog inhibitor (e.g., Oxy 200), GPR40 agonist/GPR84 antagonist (e.g., PBI-4547), neutrophil elastase inhibitor (e.g., PHP-303), integrin inhibitor (e.g., PLN-1474), TGFβ1 modulator (e.g. PRM-151), CCK receptor antagonist (e.g., proglumide), LOXL2 inhibitor (e.g., PXS-5338K, PXS-5382A), IL-11 inhibitors, MPYS protein inhibitor (e.g., cGAS/STING antagonists), kinase inhibiting RNase, membrane protein mAbs, tumor necrosis factor inhibitor, NRF2 activator (e.g., SCO 116), SSAO inhibitor (e.g., TERN 201), TRAIL2 agonist (e.g., TLY012), IL-6 receptor antagonist (e.g., TZLS 501), AOC3 inhibitor (e.g., UD-014), SSAO/VAP-1 inhibitor, TREM2); anti-oxidant (e.g., vitamin E); anti-inflammatory agents (e.g., norfloxacin, ciprofloxacin, ceftriaxone); coagulation modifiers (e.g., anti-coagulants, anti-platelet agents, pentoxifylline, vitamin K, DDAVP); dual GIP and GLP-1 receptor agonist (e.g., tirzepetide); dual GLP-1/GRA (e.g., cotadutide, ALT-801, DD 01, G49, PB-718); dual GLP-1 (e.g., CT 868); GLP-1/GRA/GIP triple agonist (e.g., HM15211); GRP120 stimulant/inflammasome modulator/PPART dual agonist (e.g., KDT501); GLP-1/FGF21 (e.g., YH25724); GLP-1 agonist (e.g., Ozempic (semaglutide sc), XW 003); selective thyroid hormone receptor-O agonist (e.g., resmetirom); apoptosis modulators (JNK-1 inhibitor (e.g., CC-90001), Peroxidase inhibitor (e.g., AZM198), ASK-1 inhibitor (e.g., CS-17919, SRT 015)); erythropoietin-stimulating agents (erythropoietin receptor agonist (e.g., cibinetide)); glucose pathway modulators (SGLT-2 inhibitor (e.g., Forxiga, Farxiga (dapagliflozin)); dual SGLT-1/2 inhibitor (e.g., licogliflozin), Glucose-6-P dehydrogenase inhibitor (e.g., fluasterone) LAPS glucagon combo (e.g., HM14320), SGLT-1 inhibitor (e.g., SGL5213)); immune modulators (TLR4 inhibitor (e.g., GBK-233), immunomodulatory polyclonal antibody (e.g., IMM-124E), TLR4 antagonist (e.g., JKB-122), CD3 monoclonal antibody (e.g.foralumab), TLR4 antagonist (e.g., JKB 133), TLR4 inhibitor (e.g., mosedipimod), Macrophage inhibitor via CD206 targeting (e.g., MT2002), TLR2/4 antagonist (e.g., VB-201, VB-703), immunomodulatory polyclonal antibody (e.g., IMM-124E)); incretin-based therapies (GLP-1 agonist (e.g., Ozempic (semaglutide sc), XW 003), GLP-1/glucagon dual receptor agonist (e.g., HM12525A), prandial insulin (e.g., ORMD 0801)); lipid modulators (AMPK Activator/Glutathione transferase (e.g., oltipraz), THR-beta agonist (e.g., resmetirom, VK2809, MGL-3745, ALG-009, ASC41, CNPT-101101, TERN 501), IBAT inhibitor (e.g., elobixibat, CJ 14199), omega-6-fatty acid (e.g., epeleuton), FASN inhibitor (e.g., TVB2640, FT 4101, FT 8225), ANGPTL3 inhibitor (e.g., vupanorsen), PNPLA3 inhibitor (e.g., AZD2693), RAS domain kinase inhibitor (e.g., BioE1115), NTCP inhibitor (e.g., bulevirtide), P2Y13 receptor agonist (e.g., CER-209), omega-3 fatty acid, HSD17β13 inhibitor; metabolism modulators (FXR agonist (e.g., Ocaliva (obeticholic acid), IOT022), recombinant variant of FGF19 (e.g., aldafermin), bi-specific FGFR1/KLB antibody (e.g., BFKB8488A), mTOT modulator (e.g., MSDC-0602K), pegylated analog of FGF21 (e.g., pegbelfermin, BMS-986171), non-bile FXR agonist (e.g., cilofexor, EDP-305, EYP 001, tropifexor, MET409, AGN-242256, AGN-242266, EDP 297, HPG 1860, MET642, RDX023, TERN 101), ACC inhibitor (e.g., firsocostat, PF-05221304), ketohexokinase inhibitor (e.g., PF-06835919), AMPK activator (e.g., PXL770, MSTM 101, O304), bile acid modulator (e.g., Albiero), FGF21 analog (e.g., B1089-100), MOTSc analog (e.g., CB4211), cyclophilin inhibitor (e.g., CRV 431), FGF19 (e.g., DEL 30), mitochondrial uncoupler (e.g., GEN 3026), FXR/GPCR dual agonist (e.g., INT-767), Cysteamine derivative (e.g., KB-GE-001), dual amylin and calcitonin receptor agonist (e.g., KBP-089), transient FXR agonist (e.g., M 1217), anti-beta-klotho (KLB)-FGFR1c receptor complex mAb (e.g., MK3655), GDF15 analog (e.g., NGM395), cyclophilin inhibitor (e.g., NV556), LXR modulator (e.g., PX 329, PX 655, PX 788), LXR inverse agonist (e.g., PX016), deuterated obeticholic acid (e.g., ZG 5216)); PPAR modulators (dual PPARα/γ agonist (e.g., elafibranor), PPAR pan agonist (e.g., lanifibranor), PPARa agonists (e.g., Parmodia), PPARγ agonist (e.g., CHS 131), MPC inhibitor (e.g., PXL065), PPAR δ/γ agonist (e.g. T3D 959)); RAAS mIMModulators (mineralocorticoid receptor antagonist (e.g., apararenone, eplerenone, spironolactone), angiotensin receptor blocker (e.g., losartan potassium)); neurotransmitter modulators (cannabinoid receptor modulator, CB1 receptor antagonist (e.g., CRB-4001, IM-102, nimacimab), TPH1 inhibitor (e.g., CU 02), GPR120 agonist (e.g., KBR2001), combination of cannabinoid and botanical anti-inflammatory compound (e.g., SCN 002)); PDE Modulator (PDE4 inhibitor (e.g., ART 648)); CYP2E1 inhibitor (e.g., SNP-610); cell therapies (e.g., HepaStem) and bromocriptine mesylate and rapid-release formulations thereof; or with other drugs beneficial for the prevention or the treatment of the above-mentioned diseases including nitroprusside and diazoxide the free-acid, free-base, and pharmaceutically acceptable salt forms of the above active agents where chemically possible.

The present disclosure includes the pharmaceutically acceptable salts of the compounds defined herein, including the pharmaceutically acceptable salts of all structural formulas, embodiments and classes defined herein. Reference to the compounds of structural Formula (I) includes the compounds of other generic structural Formulas, such as Formulas and embodiments that fall within the scope of Formula I.

### Dosages of the Compounds of Formula I

If the patient is responding, or is stable, after completion of the therapy cycle, the therapy cycle can be repeated according to the judgment of the skilled clinician. Upon completion of the therapy cycles, the patient can be continued on the compounds of Formula I at the same dose that was administered in the treatment protocol. This maintenance dose can be continued until the patient progresses or can no longer tolerate the dose (in which case the dose can be reduced and the patient can be continued on the reduced dose).

Those skilled in the art will recognize that the actual dosages and protocols for administration employed in the methods of the present disclosure may be varied according to the judgment of the skilled clinician. The actual dosage employed may be varied depending upon the requirements of the patient and the severity of the condition being treated. Determination of the proper dosage for a particular situation is within the skill of the art. A determination to vary the dosages and protocols for administration may be made after the skilled clinician considers such factors as the patient's age, condition and size, as well as the severity of the condition being treated and the response of the patient to the treatment.

The dosage regimen utilizing a compound of Formula I is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compound of Formula I is within the skill of the art. For administration to a human in the curative or prophylactic treatment of the conditions and disorders identified herein, for example, typical dosages of the compounds of the present disclosure can be about 0.05 mg/kg/day to about 50 mg/kg/day, for example at least 0.05 mg/kg, at least 0.08 mg/kg, at least 0.1 mg/kg, at least 0.2 mg/kg, at least 0.3 mg/kg, at least 0.4 mg/kg, or at least 0.5 mg/kg, and preferably 50 mg/kg or less, 40 mg/kg or less, 30 mg/kg or less, 20 mg/kg or less, or 10 mg/kg or less, which can be about 2.5 mg/day (0.5 mg/kg×5 kg) to about 5000 mg/day (50 mg/kg×100 kg), for example. For example, dosages of the compounds can be about 0.1 mg/kg/day to about 50 mg/kg/day, about 0.05 mg/kg/day to about 10 mg/kg/day, about 0.05 mg/kg/day to about 5 mg/kg/day, about 0.05 mg/kg/day to about 3 mg/kg/day, about 0.07 mg/kg/day to about 3 mg/kg/day, about 0.09 mg/kg/day to about 3 mg/kg/day, about 0.05 mg/kg/day to about 0.1 mg/kg/day, about 0.1 mg/kg/day to about 1 mg/kg/day, about 1 mg/kg/day to about 10 mg/kg/day, about 1 mg/kg/day to about 5 mg/kg/day, about 1 mg/kg/day to about 3 mg/kg/day, about 3 mg/day to about 500 mg/day, about 5 mg/day to about 250 mg/day, about 10 mg/day to about 100 mg/day, about 3 mg/day to about 10 mg/day, or about 100 mg/day to about 250 mg/day. Such doses may be administered in a single dose or may be divided into multiple doses.

### Pharmaceutical Compositions

The compounds of Formula I and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition.

Administering of the compound of Formula I to the subject includes both self-administration and administration to the patient by another person. The subject may need, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

Methods for the safe and effective administration of most of these agents are known to those skilled in the art. In addition, their administration is described in the standard literature.

If the patient is responding, or is stable, after completion of the therapy cycle, the therapy cycle can be repeated according to the judgment of the skilled clinician. Upon completion of the therapy cycles, the patient can be continued on the compounds of the disclosure at the same dose that was administered in the treatment protocol. This maintenance dose can be continued until the patient progresses or can no longer tolerate the dose (in which case the dose can be reduced and the patient can be continued on the reduced dose).

Those skilled in the art will recognize that the actual dosages and protocols for administration employed in the methods of the disclosure may be varied according to the judgment of the skilled clinician. The actual dosage employed may be varied depending upon the requirements of the patient and the severity of the condition being treated. Determination of the proper dosage for a particular situation is within the skill of the art. A determination to vary the dosages and protocols for administration may be made after the skilled clinician takes into account such factors as the patient's age, condition and size, as well as the severity of the condition being treated and the response of the patient to the treatment.

The amount and frequency of administration of the compound of formula I, and any additional agents will be regulated according to the judgment of the attending clinician (physician) considering such factors as age, condition and size of the patient as well as severity of the condition being treated.

The compounds of the invention are also useful in preparing a medicament that is useful in treating cancer.

The instant compounds are also useful in combination with therapeutic, chemotherapeutic and anti-cancer agents for the treatment of hepatic cellular carcinoma. Combinations of the presently disclosed compounds with therapeutic, chemotherapeutic and anti-cancer agents are within the scope of the disclosure. Examples of such agents can be found in Cancer Principles and Practice of Oncology by V. T. Devita and S. Hellman (editors), 9^{th} edition (May 16, 2011), Lippincott Williams & Wilkins Publishers. A person of ordinary skill in the art would be able to discern which combinations of agents would be useful based on the particular characteristics of the drugs and the cancer involved. Such agents include the following: estrogen receptor modulators, programmed cell death protein 1 (PD-1) inhibitors, programmed death-ligand 1 (PD-L1) inhibitors, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints.

The chemotherapeutic agent can be administered according to therapeutic protocols well known in the art. It will be apparent to those skilled in the art that the administration of the chemotherapeutic agent can be varied depending on the cancer being treated and the known effects of the chemotherapeutic agent on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the cancer to the administered therapeutic agents. The particular choice of chemotherapeutic agent will depend upon the diagnosis of the attending physicians and their judgment of the condition of the patient and the appropriate treatment protocol.

The initial administration can be made according to established protocols known in the art, and then, based upon the observed effects, the dosage, modes of administration and times of administration can be modified by the skilled clinician.

The determination of the order of administration, and the number of repetitions of administration of the chemotherapeutic agent during a treatment protocol, is well within the knowledge of the skilled physician after evaluation of the condition being treated and the condition of the patient.

Thus, in accordance with experience and knowledge, the practicing physician can modify each protocol for the administration of a chemotherapeutic agent according to the individual patient's needs, as the treatment proceeds. All such modifications are within the scope of the present disclosure.

The agent can be administered according to therapeutic protocols well known in the art.

It will be apparent to those skilled in the art that the administration of the anti-cancer agent can be varied depending on the cancer being treated and the known effects of the anti-cancer agent on that disease.

The initial administration can be made according to established protocols known in the art, and then, based upon the observed effects, the dosage, modes of administration and times of administration can be modified by the skilled clinician.

The particular choice of agent will depend upon the diagnosis of the attending physicians and their judgment of the condition of the patient and the appropriate treatment protocol.

The determination of the order of administration, and the number of repetitions of administration of the agent during a treatment protocol, is well within the knowledge of the skilled physician after evaluation of the cancer being treated and the condition of the patient.

Thus, in accordance with experience and knowledge, the practicing physician can modify each protocol for the administration of an anti-cancer agent according to the individual patient's needs, as the treatment proceeds. All such modifications are within the scope of the present disclosure.

The attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

The compounds, compositions and methods provided herein are useful for the treatment of cancer. Cancers that may be treated by the compounds, compositions and methods disclosed herein include, but are not limited to: Liver: hepatoma (hepatocellular carcinoma), cholangiocarcinoma, hepatoblastoma, angiosarcoma, hepatocellular adenoma, hemangioma.

PD-1 inhibitors include pembrolizumab (lambrolizumab), nivolumab and MPDL3280A. PDL-inhibitors include atezolizumab, avelumab, and durvalumab.

Further provided is a method of treating hepatic cellular carcinoma in a human patient comprising administration of a compound of Formula I or a pharmaceutically acceptable salt, hydrate or solvate thereof and a PD-1 antagonist to the patient. The compound of Formula I and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is pembrolizumab (KEYTRUDA™, Merck & Co., Inc., Kenilworth, NJ, USA), nivolumab (OPDIVO™, Bristol-Myers Squibb Company, Princeton, NJ, USA), cemiplimab (LIBTAYO™ Regeneron Pharmaceuticals, Inc., Tarrytown, NY, USA), atezolizumab (TECENTRIQ™ Genentech, San Francisco, CA, USA), durvalumab (IMFINZI™, AstraZeneca Pharmaceuticals LP, Wilmington, DE), or avelumab (BAVENCIO™, Merck KGaA, Darmstadt, Germany).

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

A compound of Formula I, or a pharmaceutically acceptable salt, hydrate or solvate thereof, may also be useful for treating cancer in combination with the following therapeutic agents: pembrolizumab (Keytruda®), abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); Ridaforolimus; sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); vorinostat (Zolinza®) and zoledronate (Zometa®), or a pharmaceutically acceptable salt thereof.

### EXAMPLES

### Methods for Making the Compounds of Present Invention

The following examples are provided so that the invention might be more fully understood. Unless otherwise indicated, the starting materials are commercially available. They should not be construed as limiting the invention in any way.

Several methods for preparing the compounds of this invention are described in the following Schemes and Examples. Starting materials and intermediates are purchased, made from known procedures, or as otherwise illustrated. Some frequently applied routes to the compounds of Formula I are also described by the Schemes as follows. In some cases, the order of carrying out the steps of reaction schemes may be varied to facilitate the reaction or to avoid unwanted reaction products. For stereoisomers, enantiomer A refers to the faster/earlier eluting enantiomer and enantiomer B refers to the slower/later eluting enantiomer at the point of separation and this nomenclature is maintained through the remainder of a synthetic sequence for a given enantiomeric series regardless of the possibility that subsequent intermediates and final compounds may have the same or opposite orders of elution.

Concentration refers to the removal of the volatile components at reduced pressure (e.g., by rotary evaporation) unless otherwise noted. All temperatures are in degrees Celsius unless otherwise noted. Mass spectra (MS) were measured by electrospray ion-mass spectroscopy (ESI) in positive ion detection mode and m/z refers to the [M+H]⁺ ion unless otherwise noted. ¹H NMR spectra were recorded at 400-600 MHz at ambient temperature unless otherwise noted. Purification by column chromatography on silica gel was accomplished using a flash chromatography system (e.g., ISCO® or Biotage®) and commercial pre-packed silica gel columns with elution using the stated solvent systems. Compounds described herein were synthesized as the racemates unless otherwise noted in the experimental procedures and compound tables.

### Preparative Examples

The compounds of the present invention can be prepared readily according to the following schemes and specific examples, or modifications thereof, using readily available starting materials, reagents and conventional synthetic procedures. In these reactions, it is also possible to make use of variants which are themselves known to those of ordinary skill in this art but are not mentioned in detail. The general procedures for making the compounds claimed in this invention can be readily understood and appreciated by one skilled in the art from viewing the following Schemes and descriptions.

### Abbreviations

Abbreviations used in the experimentals may include, but are not limited to, the following:

| | |
|---|---|
| ° C. | degrees Celsius |
| aq. | aqueous |
| ATP | adenosine triphosphate |
| CD₃OD | deuterated methanol-d4 |
| DCM | dichloromethane |
| DEA | diethylamine |
| DIPEA | N,N-diisopropylethylamine |
| DMF | dimethylformamide |
| DMSO | dimethyl sulfoxide |
| DMSO-d₆ | deuterated dimethyl sulfoxide |
| dppf | bis(diphenylphosphino)ferrocene |
| EDC | 1-ethyl-3-(3-dimethylaminopropyl)carbodiimide |
| EDTA | ethylenediaminetetraacetic acid |
| EGTA | ethylene glycol-bis(β-aminoethyl ether)- |
| | N,N,N′,N′-tetraacetic acid |
| ES | electrospray ionization |
| EtOAc | ethyl acetate |
| EtOH | ethanol |
| HATU | 1-[bis(dimethylamino)methylene]-1H-1,2,3- |
| | triazolo[4,5-b]pyridinium 3-oxide hexafluorophosphate |
| HEPES | 4-(2-Hydroxyethyl)piperazine-1-ethane-sulfonic acid |
| HMDS | hexamethyldisilazane |
| HOBT | 1H-1,2,3-benzotriazol-1-ol |
| HPLC | high performance liquid chromatography |
| IPA | isopropanol |
| L | liters |
| M | molar |
| MeCN | acetonitrile |
| MEK | methyl ethyl ketone |
| MeOH | methanol |
| MHz | megahertz |
| MSA | Methanesulfonic acid |
| min | minute |
| mL | milliliters |
| Mpa | megapascal |
| MS | mass spectroscopy |
| MTBD | 7-methyl-1,5,7-triazabicyclo(4.4.0)dec-5-ene |
| MTBE | methyl tert-butyl ether |
| NBS | N-bromosuccinimide |
| NMP | N-methyl-2-pyrrolidone |
| NMR | nuclear magnetic resonance |
| Pin | pinacolato |
| prep-TLC | Preparative thin layer chromatography |
| rac | racemic |
| sat. | saturated |
| SFC | supercritical fluid (CO₂) chromatography |
| T3P | tripropyl phosphonic anhydride |
| TCEP | tris(2-carboxyethyl)phosphine |
| TEA | triethylamine |
| TFA | trifluoroacetic acid |
| THF | tetrahydrofuran |
| TLC | thin layer chromatography |
| TR-FRET | time-resolved fluorescence energy transfer |

### General Schemes

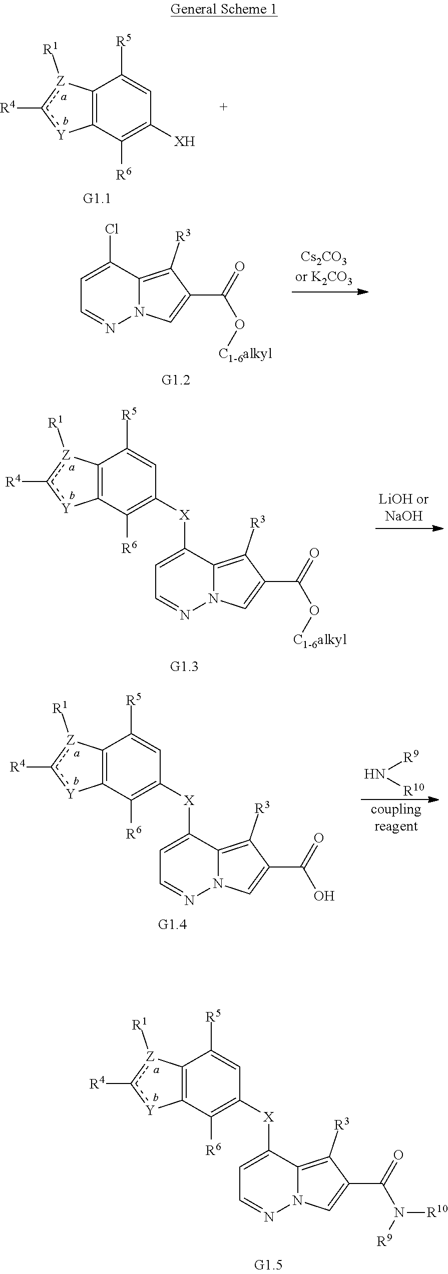

One general strategy for the synthesis of compounds of type G1.5 is via the three-step procedure shown in General Scheme 1, wherein R¹, R³, R⁴, R⁵, R⁶, R⁹, R¹⁰, X, Y, and Z are as defined in Formula I. Aryl chloride G1.2 can be combined with nucleophile G1.1 in the presence of a base such as Cs₂CO₃ or K₂CO₃ in solvents such as DMF by heating to 50° C. to form intermediate G1.3. In the second step, the ester intermediate G1.3 can then be treated with bases such as LiOH or NaOH in a solvent mixture such as THF:EtOH:H₂O to afford hydrolyzed intermediate G1.4. In the third and final step, carboxylic acid intermediate G1.4 can be coupled with an appropriate amine (HNR⁹R¹⁰) in the presence of coupling reagents such as HATU or EDC and a base such as DIPEA in a solvent such as DMF to provide products of type G1.5. Products of type G1.5 can be purified by silica gel chromatography, preparative reversed-phase HPLC, and/or chiral SFC.

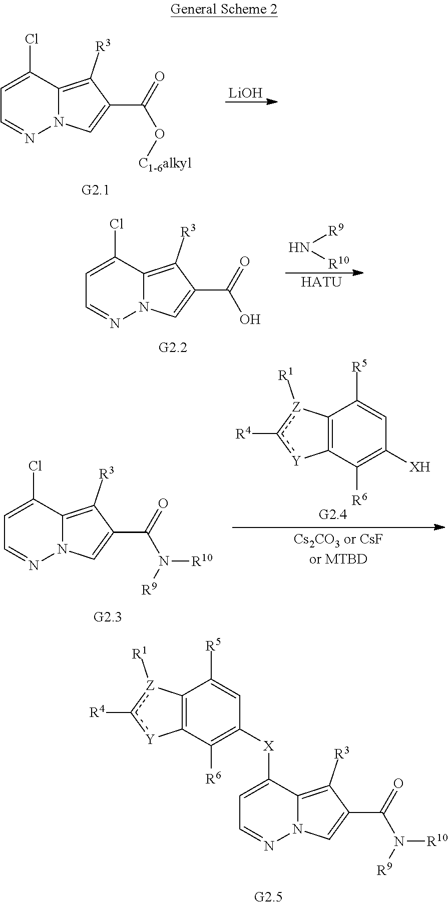

One general strategy for the synthesis of compounds of type G2.5 is via the three-step procedure shown in General Scheme 2, wherein R¹, R³, R⁴, R⁵, R⁶, R⁹, R¹⁰, X, Y, and Z are as defined in Formula I. Ester intermediate G2.1 can be treated with bases such as LiOH or NaOH in a solvent mixture such as THF:EtOH:H₂O to afford hydrolyzed intermediate G2.2. In the second step, carboxylic acid intermediate G2.2 can be coupled with an appropriate amine (HNR⁹R¹⁰) in the presence of coupling reagents such as HATU or EDC and a base such as DIPEA in a solvent such as DMF to provide intermediate G2.3. In the third and final step, aryl chloride G2.4 can then be combined with nucleophile G2.4 in the presence of a base such as Cs₂CO₃ or MTBD in solvents such as DMF by heating to 50° C. to form products of type G2.5. Products of type G2.5 can be purified by silica gel chromatography, preparative reversed-phase HPLC, and/or chiral SFC.

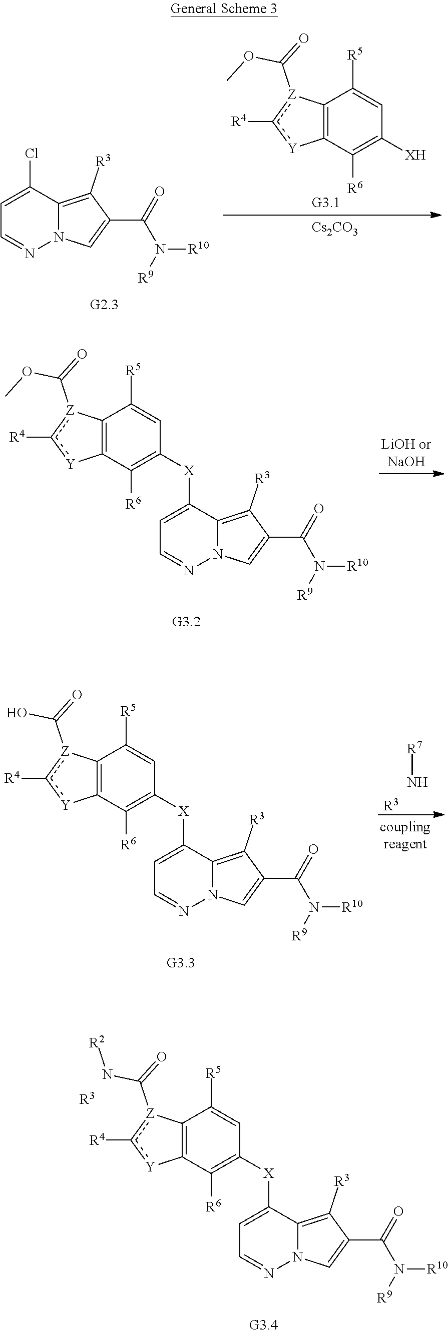

One general strategy for the synthesis of compounds of type G3.4 is via the procedure shown in General Scheme 3, wherein R³, R⁴, R⁵, R⁶, R⁷, R⁸, R⁹, R¹⁰, X, Y, and Z are as defined in Formula I. The previously described ntermediate G2.3 can be combined with nucleophile G3.1 in the presence of a base such as Cs₂CO₃ in solvents such as DMF by heating to 50° C. to form intermediate G3.2. In the fourth step, intermediate G3.2 with ester can be hydrolyzed with bases such as LiOH or NaOH in a solvent mixture such as THF:EtOH:H₂O to afford hydrolyzed intermediate G3.3. Intermediate G3.3 can then be combined with an appropriate amine (HNR⁷R⁸) in the presence of coupling reagents such as HATU or EDC and a base such as DIPEA in a solvent such as DMF to form products of type G3.4. Products of type G3.4 can be purified by silica gel chromatography, preparative reversed-phase HPLC, and/or chiral SFC.

Absolute stereochemistry of separate stereoisomers in the Examples and Intermediates was not determined unless stated otherwise in an Example or Intermediate synthesis.

### Synthesis of Intermediates

### Preparation of methyl 4-chloropyrrolo[1,2-b]pyridazine-6-carboxylate (Intermediate I-3)

Intermediate I-3 was prepared according to synthetic route outlined in Scheme 1.

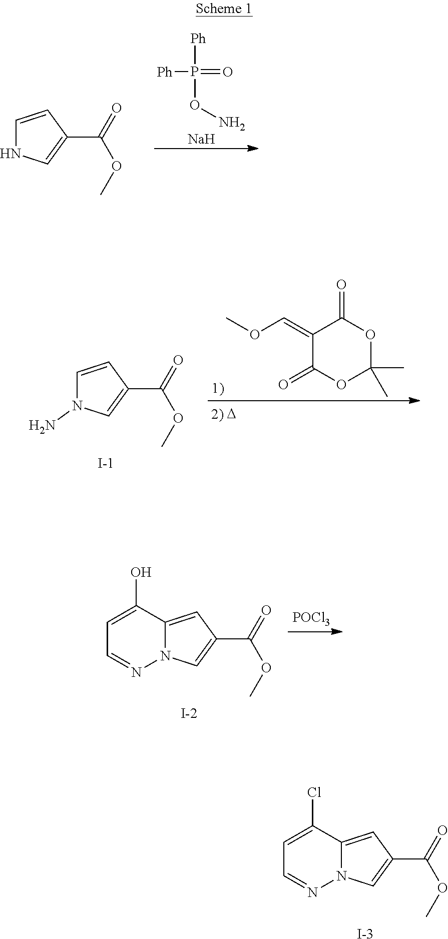

### methyl 1-amino-1H-pyrrole-3-carboxylate (I-1)

To a solution of methyl 1H-pyrrole-3-carboxylate (1.00 g, 7.99 mmol) in DMF (15 mL) was added NaH (0.416 g, 10.4 mmol) (60% in mineral oil) at 0° C., and the reaction was stirred for 0.3 hours at 25° C., then O-(2,4-dinitrophenyl)hydroxylamine (2.38 g, 12.0 mmol) was added. The reaction was stirred at 25° C. for 12 hours. The reaction mixture was poured into ice cold water (20 mL) and was extracted with EtOAc (10 mL×3). The combined organic layers were dried (Na₂SO₄), filtered and concentrated under vacuum. Column chromatography (silica gel, EtOAc in petroleum ether, gradient 0 to 30%) yielded the desired product. MS: 141 [M+1]⁺.

### methyl 4-hydroxypyrrolo[1,2-b]pyridazine-6-carboxylate (I-2)

Step 1: To a solution of methyl 1-amino-1H-pyrrole-3-carboxylate (2.20 g, 15.7 mmol) in THF (40 mL) was added 5-(methoxymethylene)-2,2-dimethyl-1,3-dioxane-4,6-dione (2.92 g, 15.7 mmol). The mixture was stirred at 25° C. for 3 hours. The reaction mixture was concentrated in vacuo, and the resultant crude material was used in the next step directly without further purification. MS: 255 [M−39]⁺.

Step 2: The crude product from step 1 was dissolved in toluene (10 mL). The reaction mixture was irradiated in the microwave at 100° C. for 90 minutes. The reaction mixture was concentrated. Column chromatography (silica gel, EtOAc in petroleum ether, gradient 0 to 50%) yielded the desired product. MS: 193 [M+1]⁺.

### methyl 4-chloropyrrolo[1,2-b]pyridazine-6-carboxylate (I-3)

To a solution of methyl 4-hydroxypyrrolo[1,2-b]pyridazine-6-carboxylate (320 mg, 1.67 mmol) in POCl₃ (0.01 mL, 0.107 mmol) was stirred at 90° C. under N₂ for 2 hours. Column chromatography (silica gel, EtOAc in petroleum ether, gradient 0 to 25%) yielded the desired product. MS: 211 [M+1]⁺.

### Preparation of ethyl 4-chloro-5-methylpyrrolo[1,2-b]pyridazine-6-carboxylate (Intermediate I-6)

Intermediate (I-6) was prepared according to synthetic route outlined in Scheme 2.

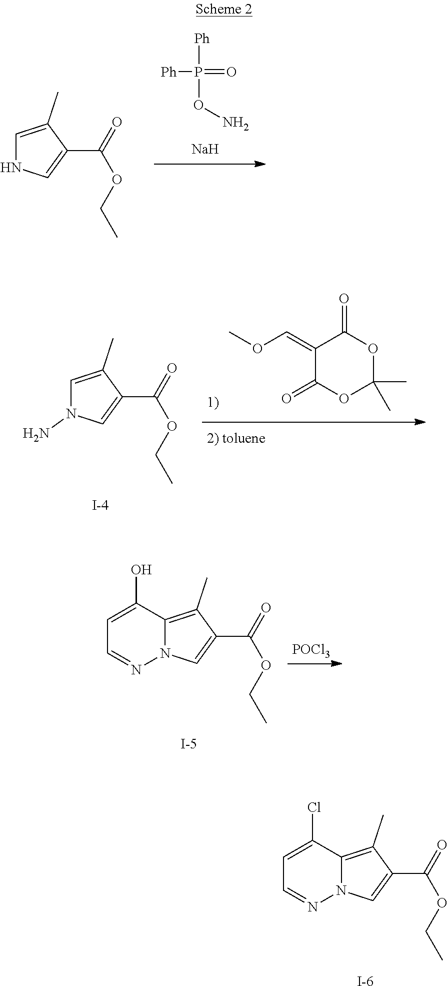

### ethyl 1-amino-4-methyl-1H-pyrrole-3-carboxylate (I-4)

NaH (66.6 g, 1.66 mol) (60% dispersed in oil) was added to DMF (4.50 L) at 0° C. To the solution, ethyl 4-methyl-1H-pyrrole-3-carboxylate (150 g, 979 mmol) was added at 0° C. After stirring under N₂ for 0.5 hours at 0° C., (aminooxy)diphenylphosphine oxide (274 g, 1.18 mol) was added to the mixture at 0° C. The resulting mixture was stirred at 25° C. for 0.5 hours. The reaction mixture was quenched with NH₄Cl (30% aq, 40 L) and extracted with MTBE (10 L×5). The combined organic phase was dried over anhydrous Na₂SO₄, filtered and concentrated in vacuo. The crude material was used directly in the next step without further purification. MS: 169 [M+1]⁺.

### ethyl 4-hydroxy-5-methylpyrrolo[1,2-b]pyridazine-6-carboxylate (I-5)

Step 1: To a solution of ethyl 1-amino-4-methyl-1H-pyrrole-3-carboxylate (335 g, 1.99 mol) in THF (3.00 L) was added 5-(methoxymethylene)-2,2-dimethyl-1,3-dioxane-4,6-dione (380 g, 1.99 mol) at 25° C. The mixture was stirred at 25° C. for 2 hours until solid precipitates. The reaction mixture was filtered and the filter cake was washed with THF (700 mL×3). The solid was dried to constant weight at 45° C. to afford desired product, which was used directly in the next step without further purification.

Step 2: The reaction uses flow chemistry. The crude product from step 1 (500 g, 1.55 mol) was charged into flow vessel 1 at 25° C. NMP (10.0 L) was charged into flow vessel 1 at 25° C. and was stirred for 5 minutes until the solution became clear. The backpressure regulator was adjusted to 0.5 Mpa and the flow rate of pump 1 was adjusted to 43 mL/min for solution. The oven was set at 170° C. and pump 1 was started. The reaction mixture was collected after running for 5 minutes and was poured into sat. Na₂CO₃ (aq, 10 L). The aqueous phase was extracted with EtOAc (30 L, 10 L, 10 L). After acidifying with 2 M HCl (aq.), aqueous phase was extracted with MTBE (5 L×2). Combined organic layers were washed with brine (2 L), dried over anhydrous Na₂SO₄, filtered and concentrated in vacuo. The crude material was used directly in the next step without further purification. MS: 221 [M+1]⁺.

### ethyl 4-chloro-5-methylpyrrolo[1,2-b]pyridazine-6-carboxylate (I-6)

Phosphorus(V) oxychloride (1.23 L, 13.2 mol) was added to MeCN (525 mL) at 25° C. Ethyl 4-hydroxy-5-methylpyrrolo[1,2-b]pyridazine-6-carboxylate (175 g, 795 mmol) and DIPEA (276 mL, 1.59 mol) were added to the mixture at 25° C. The reaction mixture was stirred at 85° C. for 12 hours under N₂. The solution was distilled in vacuum (60° C., oil pump) and poured into water (10 L). Solid NaHCO₃ was added until pH 8 and the aqueous phase was extracted with EtOAc (5 L×2). Combined organic layers were washed with brine (2 L), dried over anhydrous Na₂SO₄, filtered and concentrated in vacuo. Column chromatography (silica gel, EtOAc in hexanes, gradient 0 to 100%) yielded the desired product. MS: 239 [M+1]⁺. ¹H NMR (400 MHz, CDCl₃) δ 8.18 (s, 1H), 7.85 (d, J=4.8 Hz, 1H), 6.50 (d, J=4.8 Hz, 1H), 4.37 (q, J=7.2, Hz, 2H), 2.86 (s, 3H), 1.40 (t, J=7.2 Hz, 3H).

### Preparation of intermediate N-(bicyclo[1.1.1]pentan-1-yl)-6-hydroxy-2-methylbenzofuran-3-carboxamide (I-7)

Intermediate I-7 was prepared according to synthetic route outlined in Scheme 3.

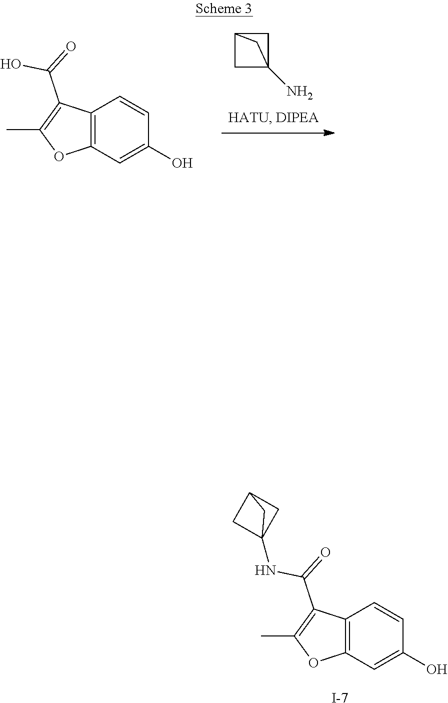

6-Hydroxy-2-methylbenzofuran-3-carboxylic acid (577 mg, 3.00 mmol), HATU (1.37 g, 3.60 mmol), and bicyclo[1.1.1]pentan-1-amine hydrochloride (431 mg, 3.60 mmol) were dissolved in EtOAc (30.0 mL). DIPEA (12.1 mL, 12.0 mmol) was added, and the reaction mixture was stirred at 25° C. for 18 hours. The final mixture was diluted with ethyl acetate and water. The organic layer was separated and washed with 1 M HCl (aq, 50 mL×2) and sat. NaHCO₃ (aq, 50 mL×2). The organic layer was dried (brine and MgSO₄), filtered, and concentrated. Column chromatography (silica gel, EtOAc/EtOH (3/1) in hexanes, gradient 10 to 100%) yielded the desired product. MS: 258 [M+1]⁺. ¹H NMR (500 MHz, DMSO-d₆) δ 9.54 (s, 1H), 8.38 (s, 1H), 7.44 (d, J=8.5 Hz, 1H), 6.86 (d, J=2.1 Hz, 1H), 6.75 (dd, J=8.5, 2.1 Hz, 1H), 2.52 (s, 3H), 2.46 (s, 1H), 2.09 (s, 6H).

TABLE 1
Using methods similar to that outlined in the sythesis of I-7, and the appropriate
starting materials, the following intermediates were prepared:

| Compound | | | Exact Mass |
|---|---|---|---|
| Number | Structure | Name | [M + 1]⁺ |
| I-8 | 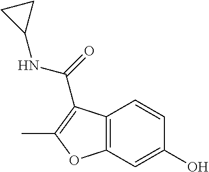 [SMILES: Cc1oc2cc(O)ccc2c1C(=O)NC1CC1] | N-cyclopropyl-6-hydroxy-2- methylbenzofuran-3- carboxamide | 232 |
| I-9 | 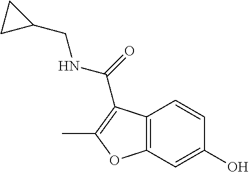 [SMILES: Cc1oc2cc(O)ccc2c1C(=O)NCC1CC1] | N-(cyclopropylmethyl)-6- hydroxy-2-methylbenzofuran- 3-carboxamide | 246 |
| I-10 | 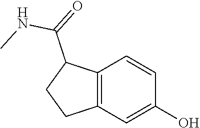 | (rac)-5-hydroxy-N-methyl-2,3- dihydro-1H-indene-1- carboxamide | 192 |

### Preparation of intermediate N-cyclobutyl-6-h, d oy-2-methylbenzofuran-3-carboxamide (I-11)

Intermediate I-11 was prepared according to synthetic route outlined in Scheme 4.

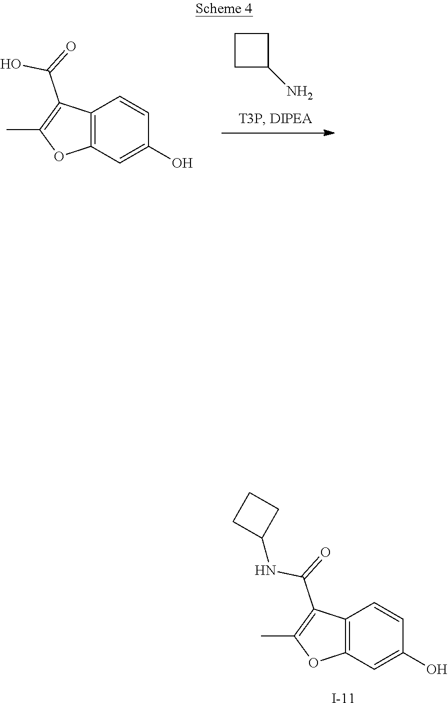

6-Hydroxy-2-methylbenzofuran-3-carboxylic acid (384 mg, 2.00 mmol) and cyclobutanamine (0.854 mL, 10.0 mmol) were dissolved in DMF (8.0 mL). T3P® (2.38 mL, 4.00 mmol) and DIPEA (14.0 mL, 8.00 mmol) were added, and the reaction mixture was stirred at 50° C. for 1.5 hours. The final mixture was diluted with ethyl acetate and washed with water (50 mL×2) and brine (50 mL). The organic layer was dried over MgSO₄, filtered, and concentrated. Column chromatography (silica gel, EtOAc in hexanes, gradient 0 to 60%) yielded the desired product. MS: 246 [M+1]⁺. ¹H NMR (500 MHz, DMSO-d₆) δ 9.55 (s, 1H), 8.11 (d, J=7.9 Hz, 1H), 7.44 (d, J=8.5 Hz, 1H), 6.87 (d, J=1.9 Hz, 1H), 6.76 (dd, J=8.5, 2.0 Hz, 1H), 4.43 (h, J=8.3 Hz, 1H), 2.53 (s, 3H), 2.27-2.18 (m, 2H), 2.14-2.02 (m, 2H), 1.71-1.61 (m, 2H).

### Preparation of intermediate 6-hydroxy-2,7-dimethylbenzofuran-3-carboxylic acid (I-14)

Intermediate (I-14) was prepared according to synthetic route outlined in Scheme 5.

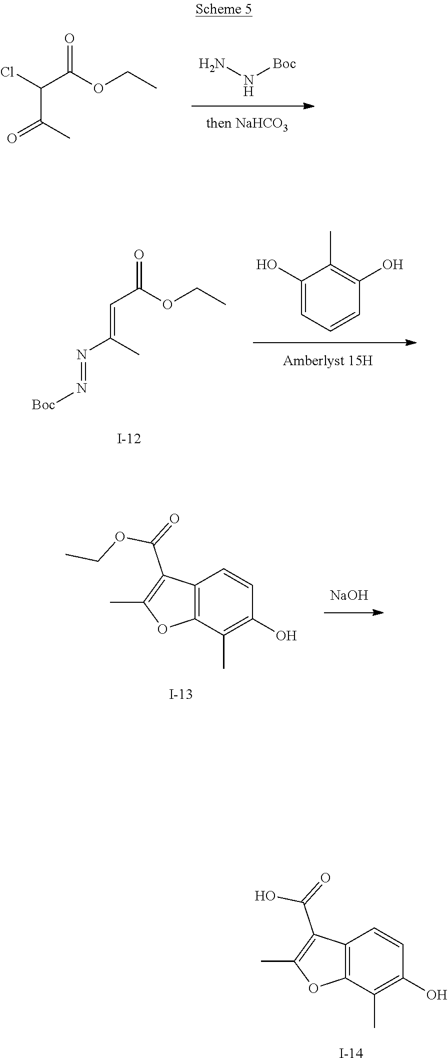

### tert-butyl (E)-2-((E)-4-ethoxy-4-oxobut-2-en-2-yl)diazene-1-carboxylate (I-12)

tert-Butyl carbazate (10 g, 76 mmol) was dissolved in THF (200 mL). Ethyl 2-chloroacetoacetate (11.2 g, 68.1 mmol) was added. The mixture was stirred at 25° C. for 12 hours. Aqueous saturated NaHCO₃ (600 mL) was added, and the reaction mixture was stirred at 25° C. for 4 hours. The reaction mixture was diluted with water then extracted with EtOAc. The organic layer was separated and dried (brine and Na₂SO₄). The mixture was filtered and then concentrated. Column chromatography (silica gel, EtOAc in petroleum ether, gradient 0 to 33%) yielded the desired product. ¹H NMR (400 MHz, CDCl₃) δ=6.89 (d, J=1.2 Hz, 1H), 4.26 (q, J=7.2 Hz, 2H), 2.22 (s, 3H), 1.60 (s, 9H), 1.32 (t, J=7.2 Hz, 3H).

### ethyl 6-hydroxy-2,7-dimethylbenzofuran-3-carboxylate (I-13)

2-methylbenzene-1,3-diol (8.5 g, 68.5 mmol) was dissolved in MeCN/acetone (95/5) (260 mL). tert-Butyl (E)-2-((E)-4-ethoxy-4-oxobut-2-en-2-yl)diazene-1-carboxylate (8.3 g, 34.2 mmol), and Amberlyst™ 15H (43.1 g, 137 mmol) were added, and the resulting reaction mixture was stirred at 25° C. for 0.5 hours. Then the mixture was stirred at 90° C. for 12 hours. The reaction mixture was diluted with water and then extracted with EtOAc. The organic layer was separated and dried (brine and Na₂SO₄). The mixture was filtered and then concentrated. Column chromatography (silica gel, EtOAc in petroleum ether, gradient 0 to 50%) yielded the desired product. MS: 235 [M+1]⁺.

### 6-hydroxy-2,7-dimethylbenzofuran-3-carboxylic acid (I-14)

Ethyl 6-hydroxy-2,7-dimethylbenzofuran-3-carboxylate (1.00 g, 4.30 mmol) and NaOH (0.68 g, 17.1 mmol) were combined. THF (10 mL) and water (10 mL) were added, and the resulting reaction mixture was stirred at 50° C. for 16 hours. The mixture was washed with EtOAc, and the aqueous phase was acidified with 6 M HCl (pH˜4). The aqueous mixture was extracted with EtOAc. The organic layer was separated and dried (brine and Na₂SO₄). The mixture was filtered and concentrated to yield the desired product. MS: 207 [M+1]⁺.

### Preparation of intermediate N-cyclobutyl-6-hydroxy-2,7-dimethylbenzofuran-3-carboxamide (I-15)

Intermediate (I-15) was prepared according to synthetic route outlined in Scheme 6.

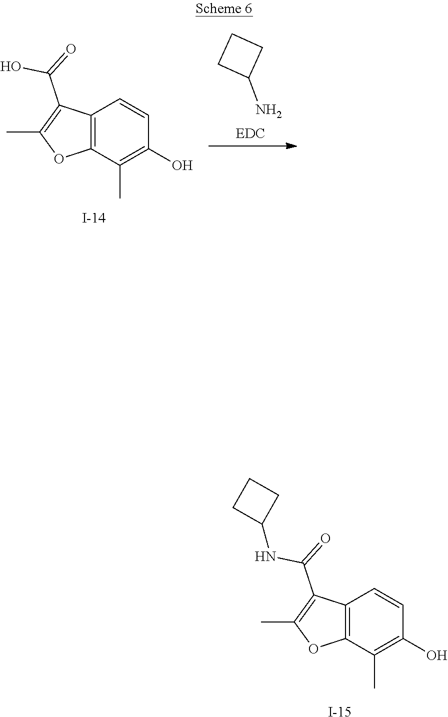

To a solution of 6-hydroxy-2,7-dimethylbenzofuran-3-carboxylic acid (250 mg, 1.21 mmol) in pyridine (6.0 mL) were added cyclobutylamine (138 mg, 1.94 mmol) and EDC (697 mg, 3.64 mmol). The reaction mixture was stirred at 25° C. for 2 hours. The reaction mixture was diluted with water and extracted with EtOAc. The organic layer was separated and dried (brine and Na₂SO₄). The mixture was filtered and concentrated. Column chromatography (silica gel, EtOAc in petroleum ether, gradient 0 to 33%) yielded the desired product. MS: 260 [M+1]⁺. ¹H NMR (400 MHz, CD₃OD) δ 7.26 (d, J=8.3 Hz, 1H), 6.77 (d, J=8.5 Hz, 1H), 4.61-4.47 (m, 1H), 2.58 (s, 3H), 2.41-2.34 (m, 2H), 2.30 (s, 3H), 2.20-2.01 (m, 2H), 1.84-1.74 (m, 2H).

TABLE 2
Using methods similar to that outlined in the synthesis of I-15, and the appropriate
starting materials, the following intermediates were prepared:

| Compound | | | Exact Mass |
|---|---|---|---|
| Number | Structure | Name | [M + 1]⁺ |
| I-16 | 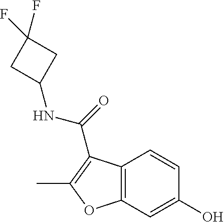 [SMILES: Cc1oc2cc(O)ccc2c1C(=O)NC1CC(F)(F)C1] | N-(3,3- difluorocyclobutyl)-6- hydroxy-2- methylbenzofuran-3- carboxamide | 282 |
| I-17 | 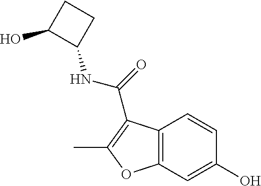 [SMILES: Cc1oc2cc(O)ccc2c1C(=O)N[C@H]1CC[C@@H]1O] | 6-hydroxy-N-((1S,2S)-2- hydroxycyclobutyl)-2- methylbenzofuran-3- carboxamide | 262 |
| I-18 | 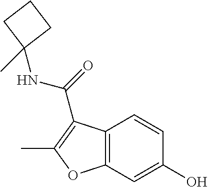 [SMILES: Cc1oc2cc(O)ccc2c1C(=O)NC1(C)CCC1] | 6-hydroxy-2-methyl-N-(1- methylcyclobutyl) benzofuran-3-carboxamide | 260 |
| I-19 | 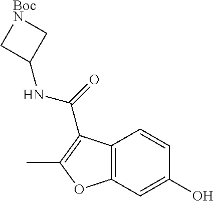 [SMILES: Cc1oc2cc(O)ccc2c1C(=O)NC1CN(C(=O)OC(C)(C)C)C1] | tert-butyl 3-(6-hydroxy-2- methylbenzofuran-3- carboxamido)azetidine-1- carboxylate | 347 |
| I-20 | 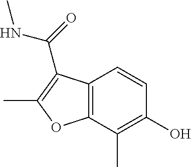 [SMILES: CNC(=O)c1c(C)oc2c(C)c(O)ccc12] | 6-hydroxy-N,2,7- trimethylbenzofuran-3- carboxamide | 220 |
| I-21 | 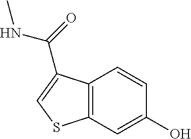 | 6-hydroxy-N- methylbenzo[b]thiophene- 3-carboxamide | 208 |

### Preparation of intermediate 7-fluoro-6-hydroxy-N,2-dimethylbenzofuran-3-carboxamide (I-25)

Intermediate (1-25) was prepared according to synthetic route outlined in Scheme 7.

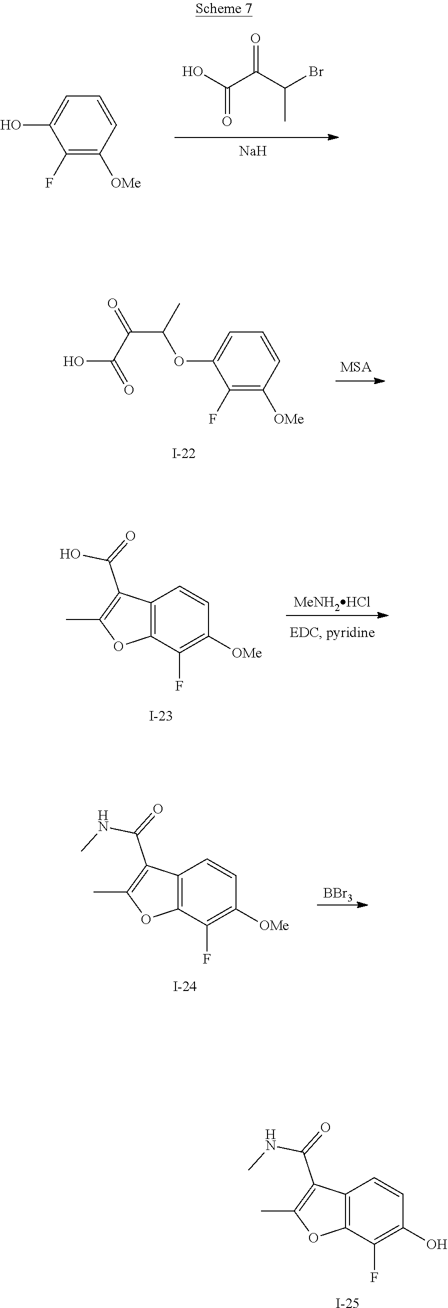

### 3-(2-fluoro-3-methoxyphenoxy)-2-oxobutanoic acid (I-22)

2-Fluoro-3-methoxyphenol (10 g, 70.4 mmol) was dissolved in THF (200 mL). The reaction mixture was cooled to 0° C. before NaH (60 wt. % in mineral oil, 5.6 g, 141 mmol) was added. After stirring at 0° C. for 30 minutes, 3-bromo-2-oxobutanoic acid (16.6 g, 91 mmol) was added. The reaction mixture was allowed to warm up to 25° C. and stirred at this temperature for 16 hours. The final reaction mixture was diluted with water and extracted with EtOAc. The aqueous phase was separated and acidified with 3 M HCl (pH˜2). The resulting aqueous mixture was extracted with EtOAc. The organic layer was dried (brine and Na₂SO₄), filtered and concentrated to yield the desired product. ¹H NMR (400 MHz, DMSO-d₆) δ 7.01 (d, J=2.0 Hz, 1H), 6.80-6.77 (m, 1H), 6.67 (t, J=7.6 Hz, 1H), 5.53 (q, J=6.8 Hz, 1H), 3.82 (s, 3H), 1.51 (d, J=6.8 Hz, 3H).

### 7-fluoro-6-methoxy-2-methylbenzofuran-3-carboxylic acid (I-23)

3-(2-Fluoro-3-methoxyphenoxy)-2-oxobutanoic acid (13.5 g, 55.7 mmol) was dissolved in DCM (270 mL). Methanesulfonic acid (5.4 mL, 84 mmol) was added, and the reaction mixture was stirred at 25° C. for 16 hours. The desired product was isolated by filtration. MS: 225 [M+1]⁺.

### 7-fluoro-6-methoxy-N,2-dimethylbenzofuran-3-carboxamide (I-24)

7-Fluoro-6-methoxy-2-methylbenzofuran-3-carboxylic acid (3.4 g, 15.2 mmol), methanamine hydrochloride (2.05 g, 30.3 mmol) and EDC (5.81 g, 30.3 mmol) were combined. Pyridine (50 mL) was added, and the reaction mixture was stirred at 25° C. for 16 hours. The reaction mixture was diluted with water and extracted with EtOAc. The organic layer was separated and dried (brine and Na₂SO₄). The mixture was filtered and concentrated. Column chromatography (silica gel, EtOAc in petroleum ether, gradient 0 to 50%) yielded the desired product. MS: 238 [M+1]⁺.

### 7-fluoro-6-hydroxy-N,2-dimethylbenzofuran-3-carboxamide (I-25)

7-Fluoro-6-methoxy-N,2-dimethylbenzofuran-3-carboxamide (2.60 g, 11.0 mmol) was dissolved in DCM (260 ml), and the resulting solution was cooled to 0° C. BBr₃ (32.9 ml, 65.8 mmol) was added at 0° C. over 3 minutes. The reaction mixture was allowed to warm to 25° C. and stirred for another 3 hours. The reaction mixture was diluted with water and extracted with DCM. The organic layer was separated and dried (brine and Na₂SO₄). The mixture was filtered and concentrated. Column chromatography (silica gel, EtOAc/EtOH (3/1) in petroleum ether, gradient 0 to 100%) yielded the desired product. MS: 224 [M+1]⁺. ¹H NMR (400 MHz, CD₃OD) δ 7.28 (d, J=8.6 Hz, 1H), 6.95-6.87 (m, 1H), 2.98-2.93 (m, 3H), 2.63 (s, 3H).

### Preparation of intermediate 6-hydroxy-2-methyl-N-(1-(2,2,2-trifluoroethyl)azetidin-3-yl)benzofuran-3-carboxamide (I-27)

Intermediate (I-27) was prepared according to synthetic route outlined in Scheme 8.

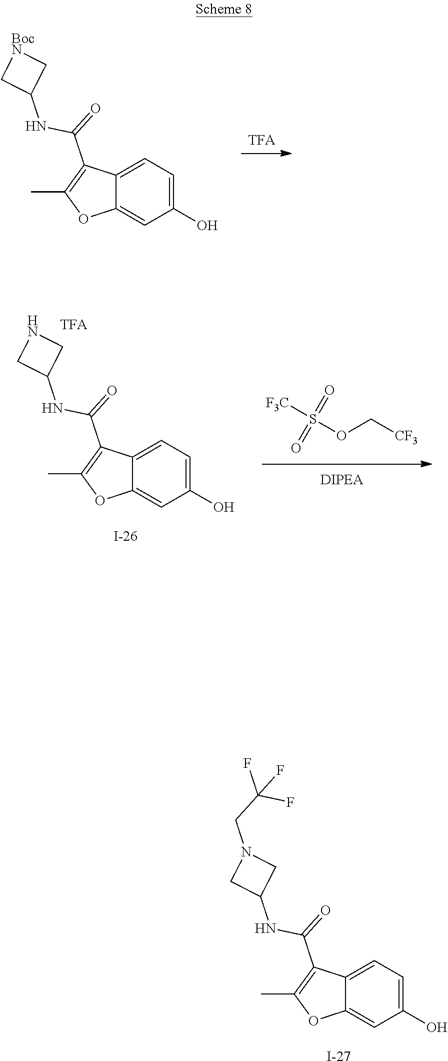

### N-(azetidin-3-yl)-6-hydroxy-2-methylbenzofuran-3-carboxamide, TFA (I-26)

To a solution of tert-butyl 3-(6-hydroxy-2-methylbenzofuran-3-carboxamido)azetidine-1-carboxylate (280 mg, 0.808 mmol) in DCM (2.20 mL) was added TFA (1.10 mL, 14.3 mmol) at 0° C. The mixture was warmed to 25° C. with stirring for 1 hour. The reaction mixture was concentrated in vacuo to yield the desired product, which was used in the next step directly without further purification. MS: 247 [M+1]⁺.

### 6-hydroxy-2-methyl-N-(1-(2,2,2-trifluoroethyl)azetidin-3-yl)benzofuran-3-carboxamide (I-27)

To a solution of N-(azetidin-3-yl)-6-hydroxy-2-methylbenzofuran-3-carboxamide, TFA (270 mg, 0.659 mmol) in 1,4-dioxane (9.00 mL) was added DIPEA (0.461 mL, 2.64 mmol) at 25° C. After stirring for 0.5 hours at 25° C., 2,2,2-trifluoroethyl trifluoromethanesulfonate (0.190 mL, 1.32 mmol) was added to the mixture at 0° C. The resulting mixture was stirred for another 16 hours at 25° C. The reaction mixture was added with water (10 mL) and extracted with EtOAc (5 mL×3). The combined organic phases were washed with brine (10 mL), dried over anhydrous Na₂SO₄, filtered and concentrated under reduced pressure. Column chromatography (silica gel, EtOAc in petroleum ether, gradient 0 to 40%) yielded the desired product. MS: 329 [M+1]⁺.

### Preparation of intermediate 6-hydroxybenzo[b]thiophene-3-carboxylic acid (I-30)

Intermediate (I-30) was prepared according to synthetic route outlined in Scheme 9.

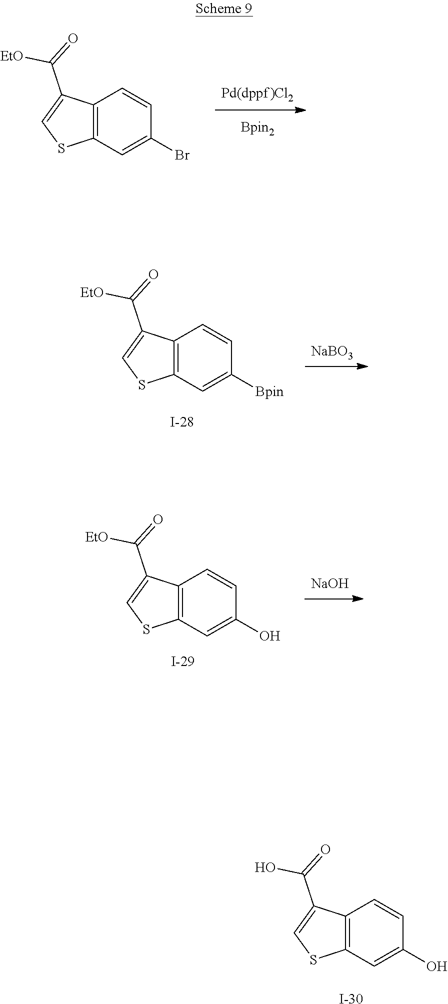

### ethyl 6-(4,4,5,5-tetramethyl-1,3,2-dioxaborolan-2-yl)benzo[b]thiophene-3-carboxylate (I-28)

Under nitrogen, ethyl 6-bromobenzo[b]thiophene-3-carboxylate (600 mg, 2.10 mmol), B₂Pin₂ (1.34 g, 5.30 mmol), PdCl₂×dppf (154 mg, 0.210 mmol), and potassium acetate (620 mg, 6.30 mmol) were combined. Dioxane (3.00 mL) was added and the resulting reaction mixture was stirred at 100° C. for 3 hours. The final reaction mixture was filtered and concentrated. Column chromatography (silica gel, EtOAc in petroleum ether, gradient 0 to 5%) yielded the desired product. MS: 333 [M+1]⁺.

### ethyl 6-hydroxybenzo[b]thiophene-3-carboxylate (I-29)

Ethyl 6-(4,4,5,5-tetramethyl-1,3,2-dioxaborolan-2-yl)benzo[b]thiophene-3-carboxylate (700 mg, 2.10 mmol) and sodium perborate tetrahydrate (1.30 g, 8.40 mmol) were combined. THF (8.00 mL) and water (8 mL) were added, and the resulting reaction mixture was stirred at 40° C. for 3 hours. The reaction mixture was diluted with water and extracted with EtOAc. The organic layer was separated and dried (brine and Na₂SO₄). The mixture was filtered and concentrated to yield the desired product, which was used in the next step without further purification. MS: 223 [M+1]⁺.

### 6-hydroxybenzo[b]thiophene-3-carboxylic acid (I-30)

Ethyl 6-hydroxybenzo[b]thiophene-3-carboxylate (450 mg, 2.00 mmol) and NaOH (162 mg, 4.05 mmol) were combined. THF (2.50 mL) and water (2.50 mL) were added, and the resulting reaction mixture was stirred at 50° C. for 3 hours. The final reaction mixture was diluted with water and extracted with EtOAc. The aqueous phase was separated and acidified with 6 M HCl (pH˜7). The resulting aqueous mixture was lyophilized to yield the desired product, which was used in the next step without further purification. ¹H NMR (400 MHz, DMSO-d₆) δ=9.66-9.66 (m, 1H), 8.41 (d, J=8.8 Hz, 1H), 7.94 (br s, 1H), 7.26 (d, J=2.0 Hz, 1H), 6.89 (dd, J=8.8, 2.4 Hz, 1H).

### Preparation of intermediate 6-hydroxy-N,3-dimethylbenzofuran-2-carboxamide (I-32)

Intermediate (I-32) was prepared according to synthetic route outlined in Scheme 10.

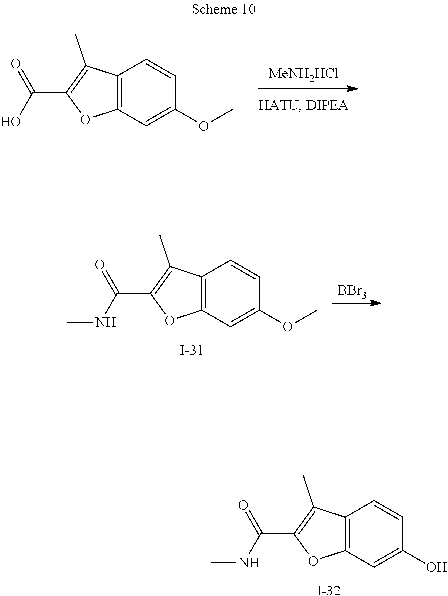

### 6-methoxy-N,3-dimethylbenzofuran-2-carboxamide (I-31)

To 6-methoxy-3-methylbenzofuran-2-carboxylic acid (412 mg, 2.00 mmol), methanamine, HCl (270 mg, 4.00 mmol), and HATU (1.14 g, 3.00 mmol) in DMF (10.0 mL), DIPEA (1.39 mL, 8.00 mmol) was added. The reaction mixture was allowed to stir at 50° C. for 2 hours. The reaction mixture was diluted with EtOAc and washed twice with water and once with brine. The combined organic fractions were dried over anhydrous MgSO₄, filtered and concentrated under reduced pressure. Column chromatography (silica gel, EtOAc in hexanes, gradient 0 to 40%) yielded the desired product. MS: 220 [M+1]⁺.

### 6-hydroxy-N,3-dimethylbenzofuran-2-carboxamide (I-32)

To 6-methoxy-N,3-dimethylbenzofuran-2-carboxamide (438 mg, 2.00 mmol) in DCM (15.0 mL), tribromoborane (8.00 mL, 1.00 M in DCM, 8.00 mmol) was added dropwise at 0° C. The mixture was stirred for 30 min. Water was added to the reaction mixture and the mixture was extracted with ethyl acetate and washed twice with water and twice with brine. The combined organic fractions were dried over anhydrous MgSO₄, filtered and concentrated under reduced pressure. Column chromatography (silica gel, EtOAc:EtOH=3:1 in hexanes, gradient 0 to 80%) yielded the desired product. MS: 206 [M+1]⁺. ¹H NMR (500 MHz, DMSO-d₆) δ 9.85 (s, 1H), 8.26 (d, J=4.6 Hz, 1H), 7.49 (d, J=8.5 Hz, 1H), 6.87 (d, J=1.9 Hz, 1H), 6.81 (dd, J=8.5, 2.0 Hz, 1H), 2.76 (d, J=4.7 Hz, 3H), 2.46 (s, 3H).

TABLE 3
Using methods similar to that outlined in the synthesis of I-32, and the appropriate
starting materials, the following intermediates were prepared:

| Compound | | | Exact Mass |
|---|---|---|---|
| Number | Structure | Name | [M + 1]⁺ |
| I-33 | 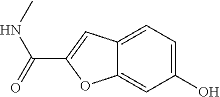 | 6-hydroxy-N- methylbenzofuran-2- carboxamide | 192 |
| I-34 | 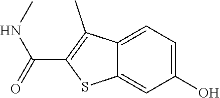 | 6-hydroxy-N,3- dimethylbenzo[b]thiophene-2- carboxamide | 222 |
| I-35 | 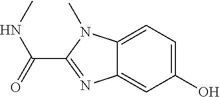 | 5-hydroxy-N,1-dimethyl-1H- benzo[d]imidazole-2- carboxamide | 206 |

### Preparation of intermediate 4-((1-fluoro-5-(methylcarbamoyl)naphthalen-2-yl)oxy)-5-methylpyrrolo[1,2-b]pyridazine-6-carboxylic acid (I-37)

Intermediate I-37 was prepared according to synthetic route outlined in Scheme 11.

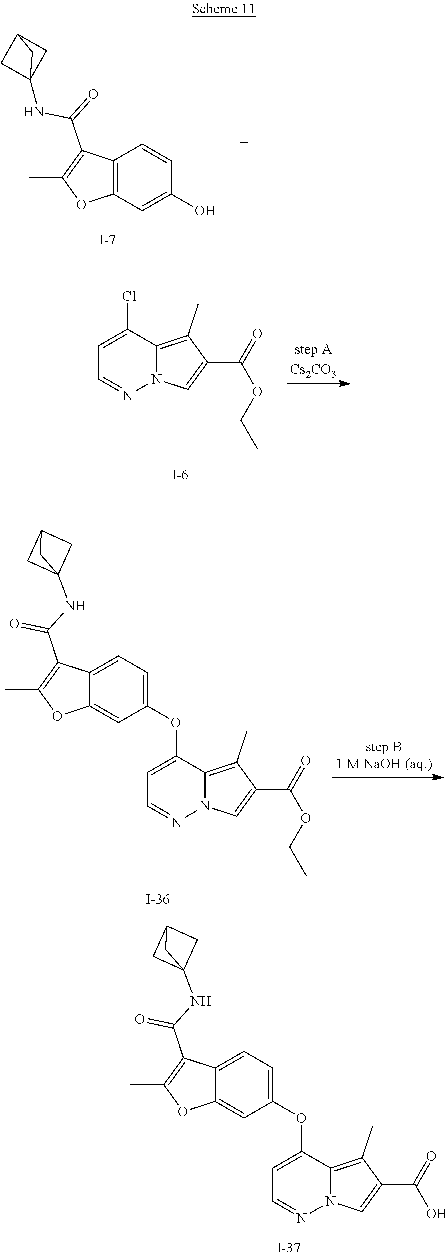

### ethyl 4-((3-(bicyclo[1.1.1]pentan-1-ylcarbamoyl)-2-methylbenzofuran-6-yl)oxy)-5-methylpyrrolo[1,2-b]pyridazine-6-carboxylate (I-36)

In a 40 mL vial, N-(bicyclo[1.1.1]pentan-1-yl)-6-hydroxy-2-methylbenzofuran-3-carboxamide (283 mg, 1.10 mmol), I-6 (239 mg, 1.00 mmol), and cesium carbonate (391 mg, 1.20 mmol) were dissolved in DMF (10.0 mL), and the reaction mixture was stirred at 80° C. for 18 hours. The crude reaction mixture was diluted with EtOAc (250 mL) and water (250 mL). The aqueous mixture was extracted with EtOAc (100 mL×2). The organic phases were combined and washed with 0.5 wt % LiCl (aq, 100 mL) and brine (aq, 100 mL×3). The organic solution was then dried (Na₂SO₄) and concentrated. Column chromatography (silica gel, MeOH in DCM, gradient 1 to 10%) yielded the desired product. MS: 460 [M+1]⁺.

### 4-((3-(bicyclo[1.1.1]pentan-1-ylcarbamoyl)-2-methylbenzofuran-6-yl)oxy)-5-methylpyrrolo[1,2-b]pyridazine-6-carboxylic acid (I-37)

To a stirring solution of ethyl 4-((3-(bicyclo[1.1.1]pentan-1-ylcarbamoyl)-2-methylbenzofuran-6-yl)oxy)-5-methylpyrrolo[1,2-b]pyridazine-6-carboxylate (460 mg, 1.00 mmol) in THF (3.00 mL) and EtOH (3.00 mL) was added 1 M NaOH (aq, 3.00 mL, 3.00 mmol), and the resulting reaction mixture was allowed to stir at 50° C. for 18 hours. The reaction mixture was diluted with water (20 mL), and then organic solvent was removed under reduced pressure. The aqueous solution was cooled in an ice bath and acidified with 1 M HCl until pH of 5. The product was isolated by filtration, washed with cold water, air dried and further dried under high vacuum. MS: 432 [M+1]⁺. ¹H NMR (500 MHz, DMSO-d₆) δ 8.62 (s, 1H), 8.17 (s, 1H), 7.98 (d, J=5.4 Hz, 1H), 7.79 (d, J=8.5 Hz, 1H), 7.67 (d, J=2.1 Hz, 1H), 7.27 (dd, J=8.5, 2.1 Hz, 1H), 5.61 (d, J=5.4 Hz, 1H), 2.74 (s, 3H), 2.62 (s, 3H), 2.48 (s, 1H), 2.13 (s, 6H).

TABLE 4
Using methods similar to that outlined in the synthesis of I-37, and appropriate
starting materials, the following intermediates were prepared:

| | | | | Exact |
|---|---|---|---|---|
| Compound | | | Reaction | Mass |
| Number | Structure | Name | Conditions | [M + 1]⁺ |
| I-38 | 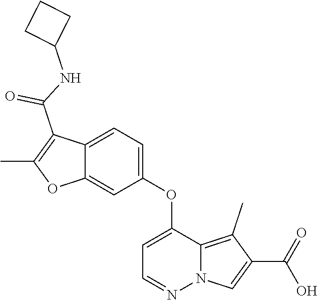 [SMILES: Cc1oc2cc(Oc3ccnn4cc(C(=O)O)c(C)c34)ccc2c1C(=O)NC1CCC1] | 4-((3- (cyclobutylcarbamoyl)-2- methylbenzofuran-6-yl) oxy)-5-methylpyrrolo [1,2-b]pyridazine-6- carboxamide | A: Cs₂CO₃ B: NaOH | 420 |
| I-39 | 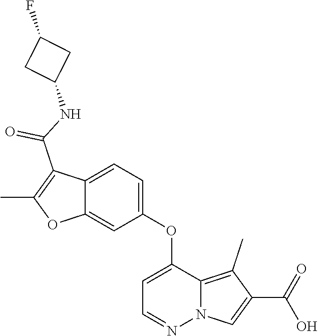 [SMILES: Cc1oc2cc(Oc3ccnn4cc(C(=O)O)c(C)c34)ccc2c1C(=O)N[C@H]1C[C@@H](F)C1] | 4-((3-(((cis)-3- fluorocyclobutyl) carbamoyl)-2- methylbenzofuran- 6-yl)oxy)-5- methylpyrrolo[1,2-b] pyridazine-6- carboxylic acid | A: Cs₂CO₃ B: NaOH | 437 |
| I-40 | 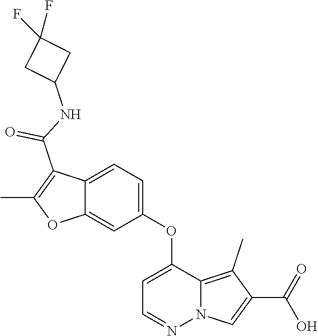 [SMILES: Cc1oc2cc(Oc3ccnn4cc(C(=O)O)c(C)c34)ccc2c1C(=O)NC1CC(F)(F)C1] | 4-((3-((3,3- difluorocyclobutyl) carbamoyl)-2- methylbenzofuran-6- yl)oxy)-5- methylpyrrolo[1,2-b] pyridazine- 6-carboxylic acid | A: Cs₂CO₃ B: NaOH | 456 |
| I-41 | 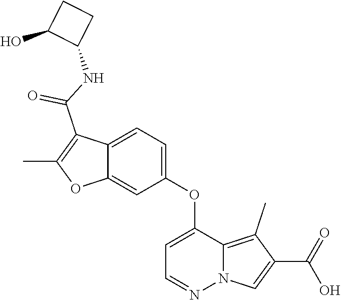 [SMILES: Cc1oc2cc(Oc3ccnn4cc(C(=O)O)c(C)c34)ccc2c1C(=O)N[C@H]1CC[C@@H]1O] | 4-((3-(((1S,2S)- 2- hydroxycyclobutyl) carbamoyl)-2- methylbenzofuran- 6-yl)oxy)-5- methylpyrrolo[1,2-b] pyridazine- 6-carboxylic acid | A: Cs₂CO₃ B: NaOH | 436 |
| I-42 | 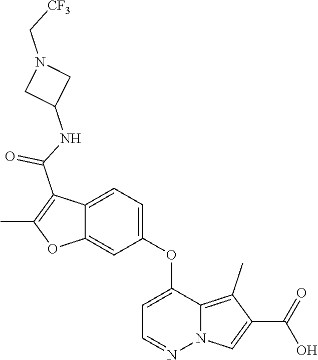 [SMILES: Cc1oc2cc(Oc3ccnn4cc(C(=O)O)c(C)c34)ccc2c1C(=O)NC1CN(CC(F)(F)F)C1] | 5-methyl-4-((2- methyl-3-((1- (2,2,2- trifluoroethyl) azetidin-3- yl)carbamoyl) benzofuran-6- yl)oxy)pyrrolo [1,2-b]pyridazine- 6-carboxylic acid | A: Cs₂CO₃ B: NaOH | 503 |
| I-43 | 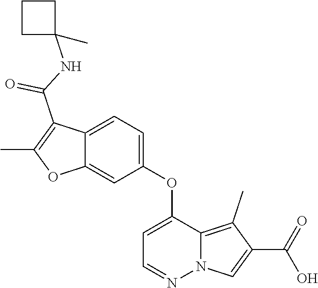 [SMILES: Cc1oc2cc(Oc3ccnn4cc(C(=O)O)c(C)c34)ccc2c1C(=O)NC1(C)CCC1] | 5-methyl-4-((2- methyl-3-((1- methylcyclobutyl) carbamoyl)benzofuran- 6-yl)oxy)pyrrolo[1,2-b] pyridazine-6- carboxylic acid | A: Cs₂CO₃ B: NaOH | 434 |
| I-44 | 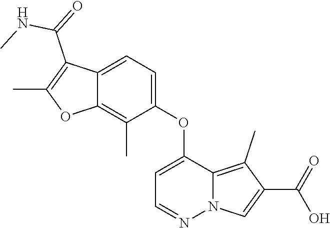 [SMILES: CNC(=O)c1c(C)oc2c(C)c(Oc3ccnn4cc(C(=O)O)c(C)c34)ccc12] | 4-((2,7-dimethyl- 3- (methylcarbamoyl) benzofuran-6- yl)oxy)-5- methylpyrrolo[1,2-b] pyridazine- 6-carboxylic acid | A: Cs₂CO₃ B: NaOH | 394 |
| I-45 | 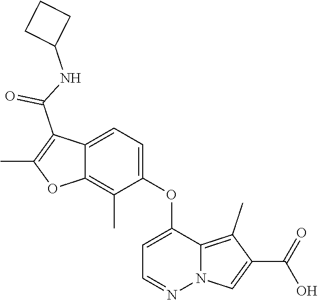 [SMILES: Cc1oc2c(C)c(Oc3ccnn4cc(C(=O)O)c(C)c34)ccc2c1C(=O)NC1CCC1] | 4-((3- (cyclobutylcarbamoyl)- 2,7-dimethylbenzofuran- 6-yl)oxy)-5- methylpyrrolo[1,2-b] pyridazine-6- carboxylic acid | A: Cs₂CO₃ B: NaOH | 434 |
| I-46 | 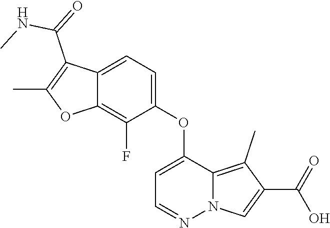 [SMILES: CNC(=O)c1c(C)oc2c(F)c(Oc3ccnn4cc(C(=O)O)c(C)c34)ccc12] | 4-((7-fluoro-2- methyl-3- (methylcarbamoyl) benzofuran-6-yl) oxy)-5- methylpyrrolo[1,2-b] pyridazine- 6-carboxylic acid | A: Cs₂CO₃ B: LiOH | 398 |
| I-47 | 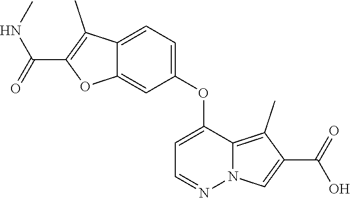 [SMILES: CNC(=O)c1oc2cc(Oc3ccnn4cc(C(=O)O)c(C)c34)ccc2c1C] | 5-methyl-4-((3- methyl-2- (methylcarbamoyl) benzofuran-6- yl)oxy)pyrrolo[1,2-b] pyridazine- 6-carboxylic acid | A: K₂CO₃ B: LiOH | 380 |
| I-48 | 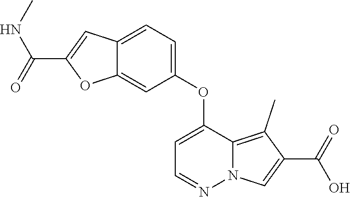 [SMILES: CNC(=O)c1cc2ccc(Oc3ccnn4cc(C(=O)O)c(C)c34)cc2o1] | 5-methyl-4-((2- (methylcarbamoyl) benzofuran-6- yl)oxy)pyrrolo[1,2-b] pyridazine- 6-carboxylic acid | A: K₂CO₃ B: LiOH | 366 |
| I-49 | 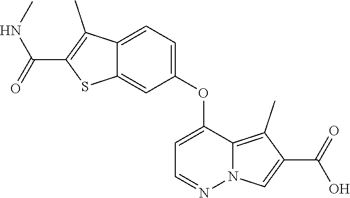 [SMILES: CNC(=O)c1sc2cc(Oc3ccnn4cc(C(=O)O)c(C)c34)ccc2c1C] | 5-methyl-4-((3- methyl-2- (methylcarbamoyl) benzo[b]thiophen-6- yl)oxy)pyrrolo[1,2-b] pyridazine- 6-carboxylic acid | A: K₂CO₃ B: LiOH | 396 |
| I-50 | 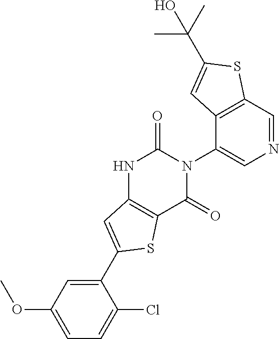 [SMILES: COc1ccc(Cl)c(-c2cc3[nH]c(=O)n(-c4cncc5sc(C(C)(C)O)cc45)c(=O)c3s2)c1] | 4-((3- ((cyclopropylmethyl) carbamoyl)-2- methylbenzofuran-6- yl)oxy)-5- methylpyrrolo[1,2-b] pyridazine- 6-carboxylic acid | A: K₂CO₃ B: NaOH | 420 |
| I-51 | 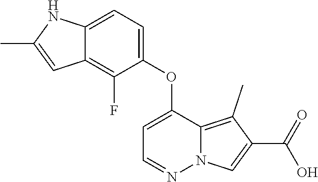 [SMILES: Cc1cc2c(F)c(Oc3ccnn4cc(C(=O)O)c(C)c34)ccc2[nH]1] | 4-((4-fluoro-2- methyl-1H- indol-5-yl)oxy)-5- methylpyrrolo[1,2-b] pyridazine- 6-carboxylic acid | A: K₂CO₃ B: NaOH | 340 |
| I-52 | 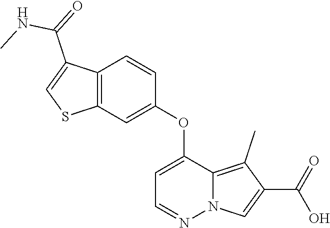 [SMILES: CNC(=O)c1csc2cc(Oc3ccnn4cc(C(=O)O)c(C)c34)ccc12] | 5-methyl-4-((3- (methylcarbamoyl) benzo[b]thiophen-6- yl)oxy)pyrrolo[1,2-b] pyridazine- 6-carboxylic acid | A: Cs₂CO₃ B: NaOH | 382 |
| I-53 | 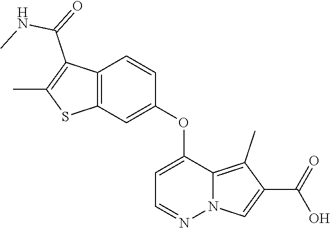 [SMILES: CNC(=O)c1c(C)sc2cc(Oc3ccnn4cc(C(=O)O)c(C)c34)ccc12] | 5-methyl-4-((2- methyl-3- (methylcarbamoyl) benzo[b]thiophen-6- yl)oxy)pyrrolo[1,2-b] pyridazine- 6-carboxylic acid | A: Cs₂CO₃ B: NaOH | 396 |
| I-54 | 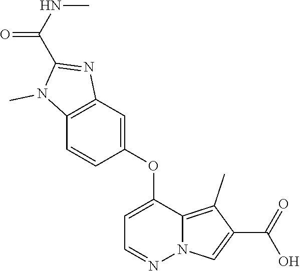 [SMILES: CNC(=O)c1nc2cc(Oc3ccnn4cc(C(=O)O)c(C)c34)ccc2n1C] | 5-methyl-4-((1- methyl-2- (methylcarbamoyl)-1H- benzo[d]imidazol-5- yl)oxy)pyrrolo[1,2-b] pyridazine- 6-carboxylic acid | A: Cs₂CO₃ B: NaOH | 380 |
| I-55 | 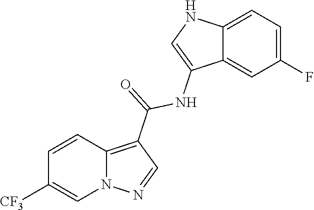 [SMILES: O=C(Nc1c[nH]c2ccc(F)cc12)c1cnn2cc(C(F)(F)F)ccc12] | (rac)-5-methyl- 4-((1- (methylcarbamoyl)-2,3- dihydro-1H-inden-5- yl)oxy)pyrrolo[1,2-b] pyridazine- 6-carboxylic acid | A: Cs₂CO₃ B: LiOH | 366 |

### Preparation of intermediates 4-((3-(((cis)2-fluorocyclobutyl)carbamoyl)-2-methylbenzofuran-6-yl)oxy)-5-methylpyrrolo[1,2-b]pyridazine-6-carboxylic acid (I-58A and I-58B)

Intermediates (I-58A and I-58) were prepared according to synthetic route outlined in Scheme 12.

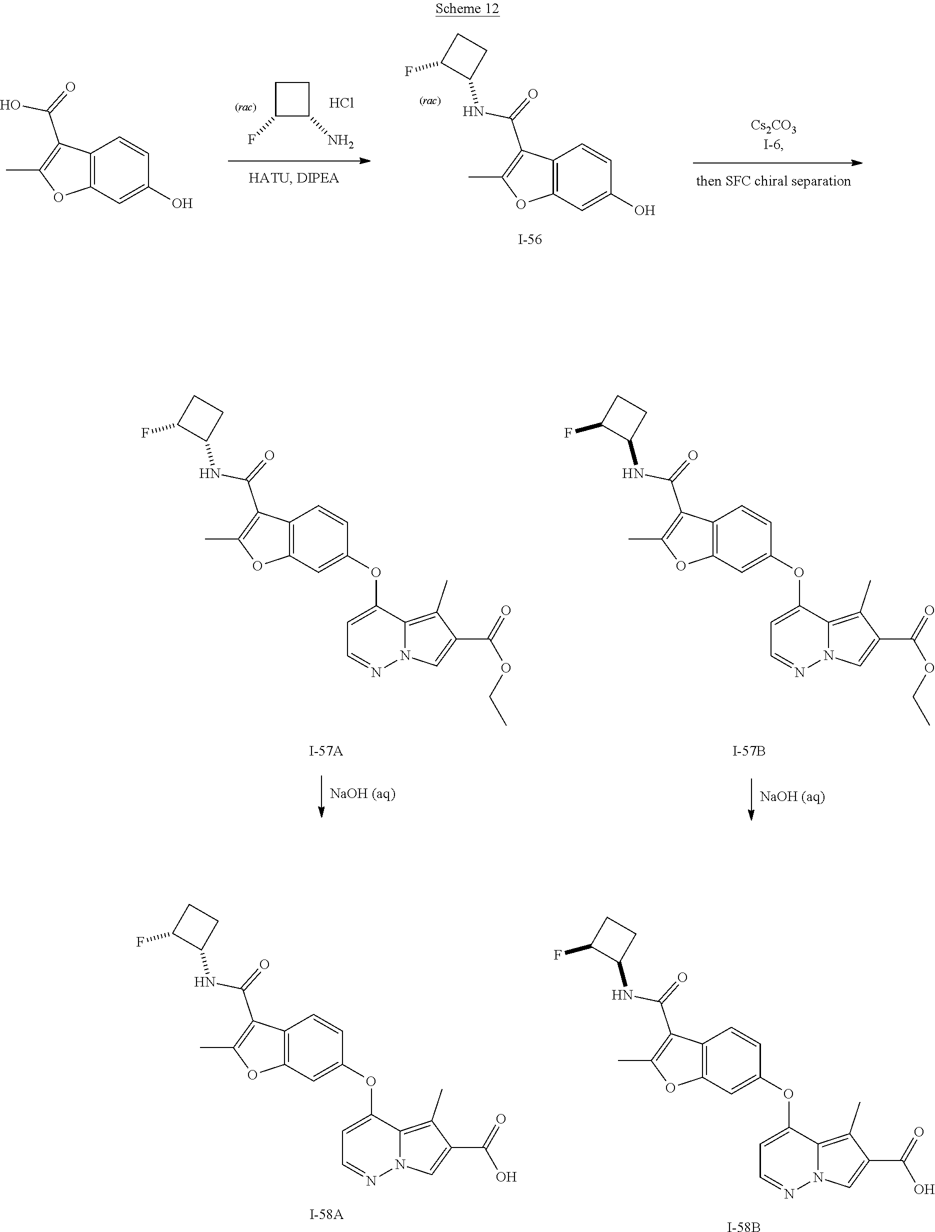

### (rac)-N-((cis)-2-fluorocyclobutyl)-6-hydroxy-2-methylbenzofuran-3-carboxamide (I-56)

6-Hydroxy-2-methylbenzofuran-3-carboxylic acid (577 mg, 3.00 mmol), HATU (1.37 g, 3.60 mmol), and cis-2-fluorocyclobutan-1-amine hydrochloride (452 mg, 3.60 mmol) were dissolved in EtOAc (30.0 mL). DIPEA (2.09 mL, 12.0 mmol) was added, and the reaction mixture was stirred at 25° C. for 18 hours. The final mixture was diluted with EtOAc and water. The organic layer was separated and washed with HCl (1 M aq, 50 mL×2) and NaHCO₃ (sat aq, 50 mL×2). The organic layer was dried (brine and Na₂SO₄), filtered, and concentrated. Column chromatography (silica gel, EtOAc/EtOH (3/1) in hexanes, gradient 10 to 100%) yielded the desired product. MS: 264 [M+1]⁺.

### ethyl 4-((3-(((cis)-2-fluorocyclobutyl)carbamoyl)-2-methylbenzofuran-6-yl)oxy)-5-methylpyrrolo[1,2-b]pyridazine-6-carboxylate (I-57A and I-57B)

In a 30 mL vial, racemic N-((cis)-2-fluorocyclobutyl)-6-hydroxy-2-methylbenzofuran-3-carboxamide (724 mg, 2.75 mmol), I-6 (597 mg, 2.50 mmol), and cesium carbonate (977 mg, 3.00 mmol) was dissolved in DMF (12.5 mL), and the resulting reaction mixture was stirred at 80° C. for 18 hours. The crude reaction mixture was diluted with EtOAc (150 mL) and water (150 mL). The aqueous mixture was extracted with EtOAc (150 mL×2). The organic phases were combined, washed with 0.5 wt % LiCl (aq, 150 mL) and brine (aq, 150 mL×3). The organic solution was then dried (Na₂SO₄) and concentrated. Column chromatography (silica gel, MeOH in DCM, gradient 1 to 10%) yielded the desired product.

The mixture of the two stereoisomers was purified by chiral SFC (SJ column, Mobile phase A: CO₂, B: 30% IPA with 0.05% NH₄OH) to afford isomer I-57A (faster eluting) and I-57B (slower eluting). Isomer I-57A: MS: 466 [M+1]⁺. Isomer I-57B: MS: 466 [M+1]⁺.

### 4-((3-(((1S,2R or 1R,2S)₂-fluorocyclobutyl)carbamoyl)-2-methylbenzofuran-6-yl)oxy)-5-methylpyrrolo[1,2-b]pyridazine-6-carboxylic acid (I-58A)

To a stirring solution of I-57A (500 mg, 1.07 mmol) in THF (3.58 mL) and ethanol (3.58 mL) was added 1 M NaOH (aq, 3.22 mL, 3.22 mmol), and the resulting reaction mixture was allowed to stir at 50° C. for 24 hours. The reaction mixture was diluted with water (20 mL), and the organic solvents were removed under reduced pressure. The aqueous solution was cooled in an ice bath and acidified with 1 M HCl until pH of 5. The product was isolated by extraction with chloroform/IPA (3/1) and the combined organic fractions were dried (MgSO₄), and filtered. The solvent was evaporated under reduced pressure. After further drying under high vacuum, the desired product was obtained. MS: 438 [M+1]⁺. ¹H NMR (500 MHz, DMSO-d₆) δ 12.50 (s, 1H), 8.44 (d, J=7.7 Hz, 1H), 8.17 (s, 1H), 7.98 (d, J=5.4 Hz, 1H), 7.77 (d, J=8.5 Hz, 1H), 7.69 (d, J=2.0 Hz, 1H), 7.29 (dd, J=8.4, 2.1 Hz, 1H), 5.62 (d, J=5.4 Hz, 1H), 5.20 (d, J=56.5 Hz, 1H), 4.74-4.61 (m, 1H), 2.75 (s, 3H), 2.64 (s, 3H), 2.30-2.11 (m, 4H).

### 4-((3-(((1R,2S or 1S,2R)₂-fluorocyclobutyl)carbamoyl)-2-methylbenzofuran-6-yl)oxy)-5-methylpyrrolo[1,2-b]pyridazine-6-carboxylic acid (I-58B)

I-58B was prepared using methods similar to that outlined in the synthesis of I-58A, and starting with I-57B in place of I-57A. MS: 438 [M+1]⁺. ¹H NMR (500 MHz, DMSO-d₆) δ 12.50 (s, 1H), 8.44 (d, J=7.9 Hz, 1H), 8.17 (s, 1H), 7.98 (d, J=5.4 Hz, 1H), 7.77 (d, J=8.5 Hz, 1H), 7.69 (d, J=2.1 Hz, 1H), 7.29 (dd, J=8.5, 2.1 Hz, 1H), 5.62 (d, J=5.4 Hz, 1H), 5.20 (d, J=56.6 Hz, 1H), 4.71-4.58 (m, 1H), 2.75 (s, 3H), 2.64 (s, 3H), 2.33-2.09 (m, 4H).

TABLE 5
Using methods similar to that outlined in the synthesis of I-58, and the appropriate
starting materials, the following intermediates were prepared:

| Compound | | | Exact Mass |
|---|---|---|---|
| Number | Structure | Name | [M + 1]⁺ |
| I-59A | 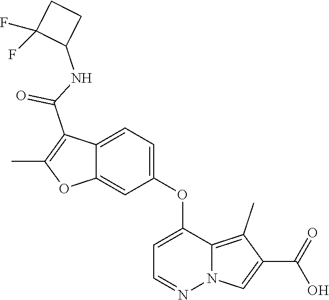 [SMILES: Cc1oc2cc(Oc3ccnn4cc(C(=O)O)c(C)c34)ccc2c1C(=O)NC1CCC1(F)F] | (R or S)-4-((3-((2,2- difluorocyclobutyl)carbamoyl)- 2-methylbenzofuran-6- yl)oxy)-5-methylpyrrolo[1,2-b] pyridazine- 6-carboxylic acid | 456 |
| I-59B | 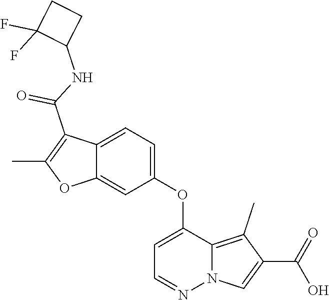 [SMILES: Cc1oc2cc(Oc3ccnn4cc(C(=O)O)c(C)c34)ccc2c1C(=O)NC1CCC1(F)F] | (S or R)-4-((3-((2,2- difluorocyclobutyl)carbamoyl) 2-methylbenzofuran-6- yl)oxy)-5-methylpyrrolo[1,2-b] pyridazine- 6-carboxylic acid | 456 |

### Preparation of intermediate 4-((1-(ethylcarbamoyl)-2-methyl-1H-indol-5-yl)oxy)-5-methylpyrrolo[1,2-b]pyridazine-6-carboxylic acid (I-62)

Intermediate I-62 was prepared according to synthetic route outlined in Scheme 13.

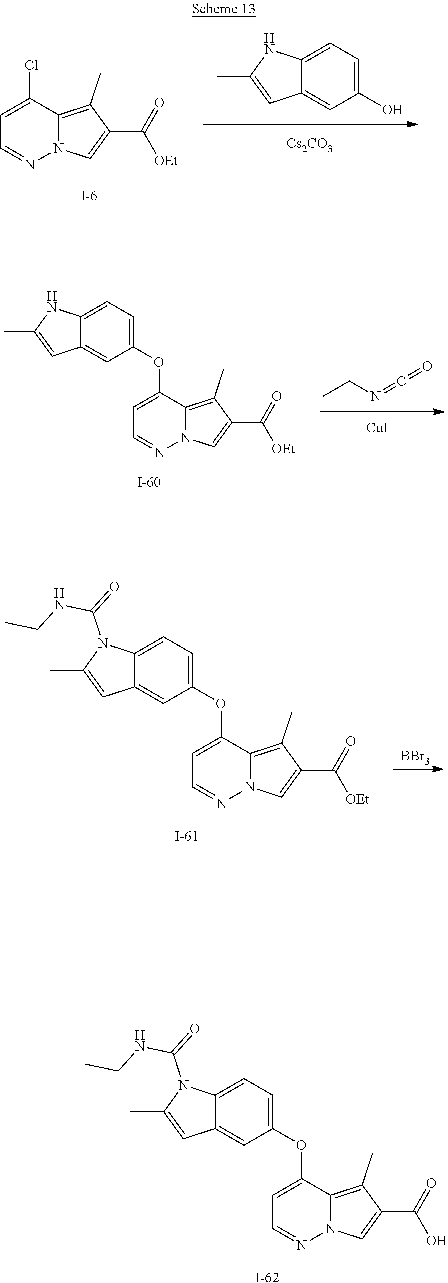

### ethyl 5-methyl-4-((2-methyl-1H-indol-5-yl)oxy)pyrrolo[1,2-b]pyridazine-6-carboxylate (I-60)

A mixture of 2-methyl-1H-indol-5-ol (1.00 g, 6.79 mmol), ethyl 4-chloro-5-methylpyrrolo[1,2-b]pyridazine-6-carboxylate (1.62 g, 6.79 mmol) and Cs₂CO₃ (4.43 g, 13.6 mmol) in DMF (25 ml) was stirred at 90° C. for 3 hours. The reaction mixture was quenched with water (50 mL) and extracted with EtOAc (25 mL×3). The combined organic phases were washed with brine (25 mL), dried over anhydrous Na₂SO₄, filtered and concentrated under reduced pressure. Column chromatography (silica gel, EtOAc in petroleum ether, gradient 15 to 30%) yielded the desired product. MS: 350 [M+1]⁺.

### ethyl 4-((1-(ethylcarbamoyl)-2-methyl-1H-indol-5-yl)oxy)-5-methylpyrrolo[1,2-b]pyridazine-6-carboxylate (I-61)

To a mixture of ethyl 5-methyl-4-((2-methyl-1H-indol-5-yl)oxy)pyrrolo[1,2-b]pyridazine-6-carboxylate (50.0 mg, 0.143 mmol) and copper(I) iodide (8.18 mg, 0.043 mmol) in DMSO (0.500 mL) was added isocyanatoethane (0.113 mL, 1.43 mmol) under N₂. The mixture was stirred at 25° C. for 2 hours under N₂. The reaction mixture was quenched with H₂O (2 mL) and extracted with EtOAc (2 mL×3). The combined organic phases were washed with brine (1 mL), dried over anhydrous Na₂SO₄, filtered and concentrated under reduced pressure. The residue was purified by prep-TLC (silica gel, ethyl acetate/petroleum ether=1/2, v/v) to give the desired product. MS: 421 [M+1]⁺.

### 4-((1-(ethylcarbamoyl)-2-methyl-1H-indol-5-yl)oxy)-5-methylpyrrolo[1,2-b]pyridazine-6-carboxylic acid (I-62)

To a solution of ethyl 4-((1-(ethylcarbamoyl)-2-methyl-1H-indol-5-yl)oxy)-5-methylpyrrolo[1,2-b]pyridazine-6-carboxylate (100 mg, 0.238 mmol) in DCM (3 mL) was added BBr₃ (1.00 mL, 10.6 mmol) at 0° C. The resulting mixture was stirred at 25° C. for 2 hours. The reaction mixture was quenched with water (2 mL) at 0° C. and lyophilized to yield the desired product, which was used directly without further purification. MS: 393 [M+1]⁺.

### Preparation of intermediate 5-methyl-4-((2-methyl-1-(methylcarbamoyl)-1H-indol-5-yl)oxy)pyrrolo[1,2-b]pyridazine-6-carboxylic acid (I-3)

Intermediate I-3 was prepared according to synthetic route outlined in Scheme 1

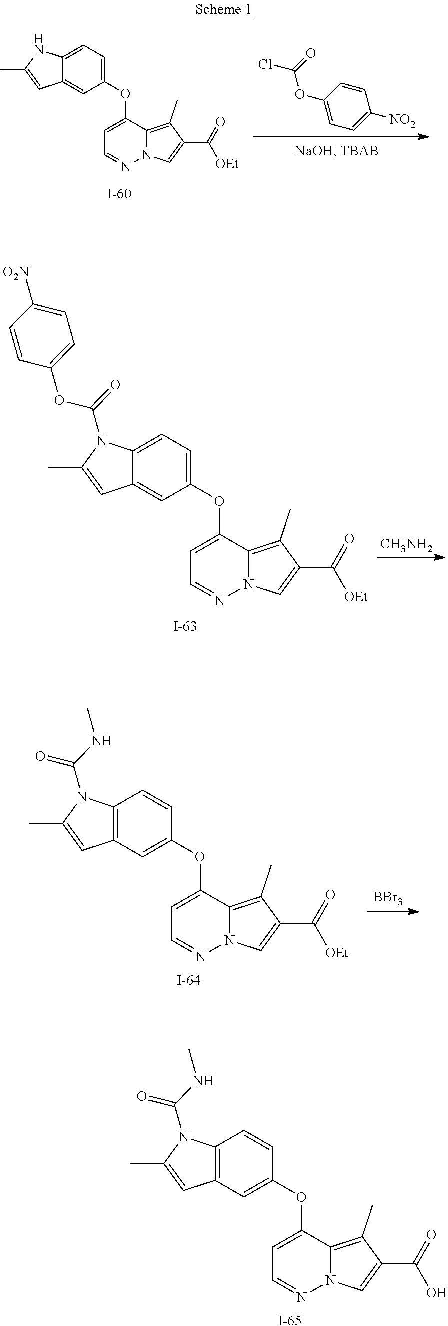

### ethyl 5-methyl-4-((2-methyl-1-((4-nitrophenoxy)carbonyl)-1H-indol-5-yl)oxy)pyrrolo[1,2-b]pyridazine-6-carboxylate (I-1)

Into DCM (2.25 L, 10 V) were added ethyl 5-methyl-4-[(2-methyl-1H-indol-5-yl)oxy]pyrrolo[1,2-b]pyridazine-6-carboxylate (225 g, 644 mmol) sodium hydroxide (77.3 g, 1932 mmol) and 4-nitrophenyl carbonochloridate (194.7 g, 966 mmol) at RT. The mixture was stirred for 2 hours. Analysis by LC/MS confirmed the formation of the desired intermediate I-1, which was carried forward directly to the next step without further manipulation.

### ethyl 5-methyl-4-((2-methyl-1-(methylcarbamoyl)-1H-indol-5-yl)oxy)pyrrolo[1,2-b]pyridazine-6-carboxylate (I-2)

Methylamine (181 mL, 5831 mmol) was added dropwise into the solution of I-1 at room temperature, and stirred for 2 hours. The resulting mixture was washed with water (3 L, 10 V) twice. The residue was purified by silica gel column chromatography, eluted with PE:EA (10:1 to 3:1) to afford product I-2.

### 5-methyl-4-((2-methyl-1-(methylcarbamoyl)-1H-indol-5-yl)oxy)pyrrolo[1,2-b]pyridazine-6-carboxylic acid (I-3)

To an ice-cooled solution of ethyl 5-methyl-4-{[2-methyl-1-(methylcarbamoyl)indol-5-yl]oxy}pyrrolo[1,2-b]pyridazine-6-carboxylate (185 g, 455.169 mmol, 1 eq.) in DCM (9250 mL, 50 V) was added boron tribromide (513.13 g, 2048.260 mmol, 4.5 eq.) and the resulting reaction mixture was warmed to room temperature and stirred for 16 hours. Take sample for HPLC. The reaction was quenched by adding dropwise to ice water (1.85 L, 10 V). Concentrated under reduced pressure to dryness. Charged the THF (925 mL, 5 V) and 1,2-dichloroethane (2775 mL, 15 V), stirred for 2 h at room temperature, filtered and dried to afford 5-methyl-4-((2-methyl-1-(methylcarbamoyl)-1H-indol-5-yl)oxy)pyrrolo[1,2-b]pyridazine-6-carboxylic acid (I-3). MS: 379 [M+1]⁺. ¹H NMR (400 MHz, DMSO-d₆,) δ 12.45 (s, 1H), 8.26 (q, J=4.4 Hz, 1H), 8.15 (d, J=1.9 Hz, 1H), 7.95 (d, J=5.4 Hz, 1H), 7.70 (d, J=8.9 Hz, 1H), 7.42 (d, J=2.4 Hz, 1H), 7.09 (dd, J=8.8, 2.5 Hz, 1H), 6.42 (d, J=5.0 Hz, 1H), 5.55 (d, J=5.4 Hz, 1H), 2.90 (d, J=4.5 Hz, 3H), 2.76 (d, J=4.6 Hz, 3H), 2.53 (s, 1H).

### Preparation of intermediate 1-(4-chloro-5-methylpyrrolo[1,2-b]pyridazine-6-carbonyl)azetidine-3-carbonitrile (I-67)

Intermediate I-67 was prepared according to synthetic route outlined in Scheme 15.

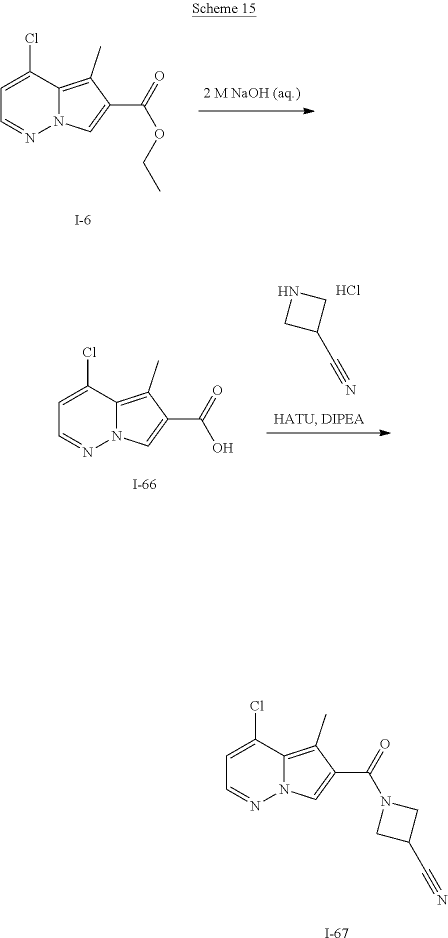

### 4-chloro-5-methylpyrrolo[1,2-b]pyridazine-6-carboxylic acid (I-66)

To a stirring solution of I-6 (2.0 g, 8.38 mmol) in THF (50.0 mL) and ethanol (50.0 mL) was added 2 M NaOH (aq, 12.6 mL, 25.1 mmol), and the resulting reaction mixture was allowed to stir at 50° C. for 16 hours. The reaction mixture was quenched with 1 M HCl (aq, 20 mL), and the mixture was extracted with EtOAc (20 mL×2). The combined organic fractions were dried (MgSO₄), filtered and the solvent was evaporated under reduced pressure to afford the desired product, which was used in the next step without further purification. MS: 211 [M+1]⁺.

### 1-(4-chloro-5-methylpyrrolo[1,2-b]pyridazine-6-carbonyl)azetidine-3-carbonitrile (I-67)

A 100 mL round bottom flask was charged with 4-chloro-5-methylpyrrolo[1,2-b]pyridazine-6-carboxylic acid (1.14 g, 5.41 mmol), azetidine-3-carbonitrile HCl (770 mg, 6.49 mmol), HATU (2.47 g, 6.49 mmol) and DMF (18.0 mL). DIPEA (2.83 mL, 16.2 mmol) was added, and the resulting reaction mixture was stirred at 25° C. for 16 hours. The mixture was concentrated, diluted with EtOAc (20 mL), washed with aqueous lithium chloride (1 M, 20 mL×2), dried (MgSO₄), and filtered. The solvent was evaporated under reduced pressure. Column chromatography (silica gel, EtOAc in hexanes, gradient 0 to 50%) yielded the desired product. MS: 275 [M+1]⁺. ¹H NMR (600 MHz, DMSO-d₆) δ 8.25 (s, 1H), 8.12 (d, J=4.9 Hz, 1H), 6.85 (d, J=4.9 Hz, 1H), 4.55 (d, J=39.2 Hz, 2H), 4.34 (s, 1H), 4.17 (s, 1H), 3.87-3.76 (m, 1H), 2.67 (s, 3H).

TABLE 6
Using methods similar to that outlined in the synthesis of I-64, and the appropriate
starting materials, the following intermediates were prepared:

| Compound | | | Exact Mass |
|---|---|---|---|
| Number | Structure | Name | [M + 1]⁺ |
| I-68 | 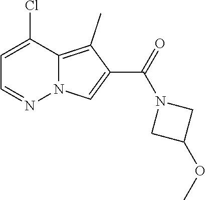 [SMILES: COC1CN(C(=O)c2cn3nccc(Cl)c3c2C)C1] | (4-chloro-5- methylpyrrolo[1,2- b]pyridazin-6-yl)(3- methoxyazetidin-1- yl)methanone | 280 |
| I-69 | 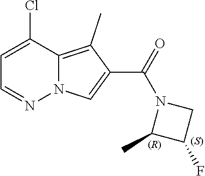 | (4-chloro-5- methylpyrrolo[1,2- b]pyridazin-6-yl)((2R,3S)-3- fluoro-2-methylazetidin-1- yl)methanone | 282 |
| I-70 | 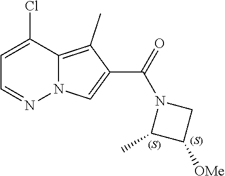 | (4-chloro-5- methylpyrrolo[1,2- b]pyridazin-6-yl)((2S,3S)-3- methoxy-2-methylazetidin-1- yl)methanone | 294 |
| I-71 | 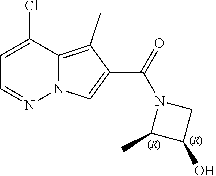 | (4-chloro-5- methylpyrrolo[1,2- b]pyridazin-6-yl)((2R,3R)-3- hydroxy-2-methylazetidin-1- yl)methanone | 280 |
| I-72 | 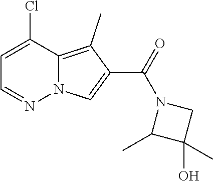 [SMILES: Cc1c(C(=O)N2CC(C)(O)C2C)cn2nccc(Cl)c12] | (4-chloro-5- methylpyrrolo[1,2- b]pyridazin-6-yl)(3-hydroxy- 2,3-dimethylazetidin-1- yl)methanone | 294 |

### Preparation of intermediate (R)-(4-chloro-5-methylpyrrolo[1,2-b]pyridazin-6-yl)(3-hydroxy-3-methylpyrrolidin-1-yl)methanone (I-73)

Intermediate 1-70 was prepared according to synthetic route outlined in Scheme 16.

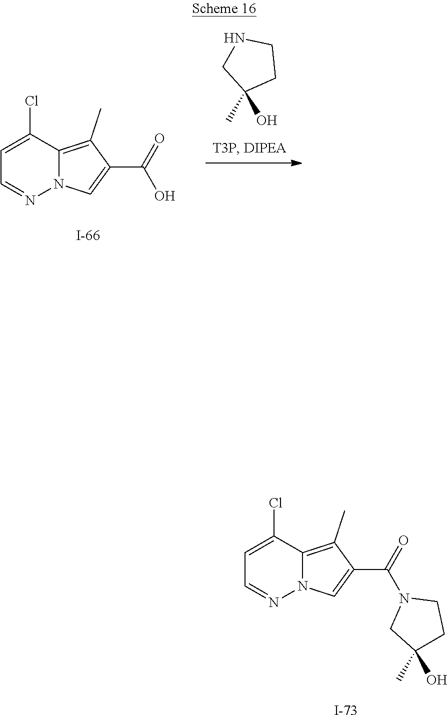

4-Chloro-5-methylpyrrolo[1,2-b]pyridazine-6-carboxylic acid (527 mg, 2.50 mmol) and (R)-3-methylpyrrolidin-3-ol HCl (460 mg, 3.34 mmol) were dissolved in DMF (20.0 mL). T3P® (2.21 mL, 3.75 mmol) and DIPEA (0.870 mL, 5.00 mmol) were added, and the resulting reaction mixture was stirred at 50° C. for 16 hours. The mixture was concentrated, diluted with EtOAc (20 mL), washed with water and brine (20 mL×2). The organic layer was dried (MgSO₄), and filtered. The solvent was evaporated under reduced pressure. Column chromatography (silica gel, EtOAc/EtOH (3/1) in hexanes, gradient 0 to 100%) yielded the desired product. MS: 294 [M+1]⁺. ¹H NMR (600 MHz, CDCl₃) δ 7.87-7.78 (m, 2H), 6.53 (d, J=4.8 Hz, 1H), 3.95-3.38 (m, 4H), 2.67 (s, 3H), 2.09-1.92 (m, 2H), 1.56-1.40 (m, 3H).

TABLE 7
Using methods similar to that outlined in the synthesis of I-73, and the appropriate
starting materials, the following intermediates were prepared:

| | | | Exact Mass |
|---|---|---|---|
| Compound Number | Structure | Name | [M + 1]⁺ |
| I-74 | 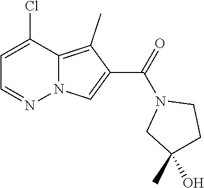 [SMILES: Cc1c(C(=O)N2CC[C@](C)(O)C2)cn2nccc(Cl)c12] | (S)-(4-chloro-5- methylpyrrolo[1,2- b]pyridazin-6-yl)(3- hydroxy-3- methylpyrrolidin-1- yl)methanone | 294 |

### Preparation of intermediate I-6-(R)-((6-(3-hydroxy-3-methylpyrrolidine-1-carbonyl)-5-methylpyrrolo[1,2-b]pyridazin-4-yl)oxy)-2-methylbenzofuran-3-carboxylic acid (I-76)

Intermediate I-76 was prepared according to synthetic route outlined in Scheme 17.

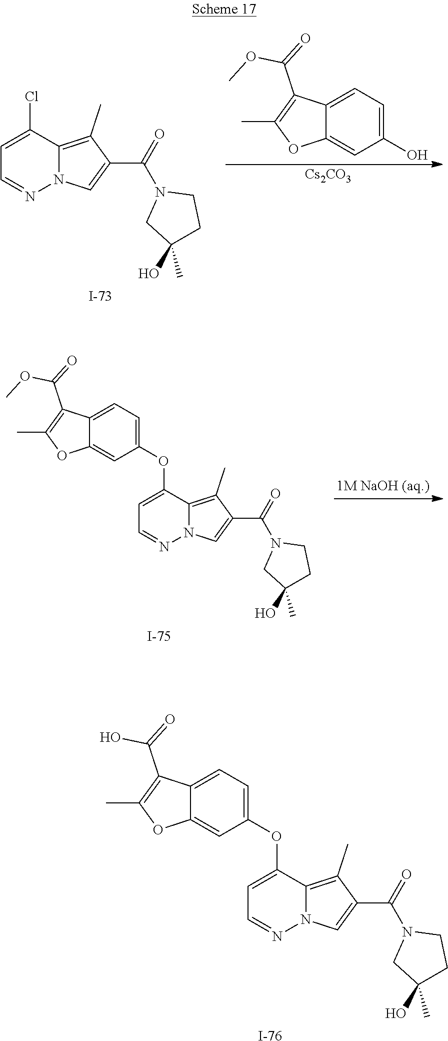

### methyl(R)-6-((6-(3-hydroxy-3-methylpyrrolidine-1-carbonyl)-5-methylpyrrolo[1,2-b]pyridazin-4-yl)oxy)-2-methylbenzofuran-3-carboxylate (I-75)

Under air, a 30 mL vial equipped with a magnetic stir bar was charged with methyl 6-hydroxy-2-methylbenzofuran-3-carboxylate (495 mg, 2.40 mmol), (R)-(4-chloro-5-methylpyrrolo[1,2-b]pyridazin-6-yl)(3-hydroxy-3-methylpyrrolidin-1-yl)methanone (588 mg, 2.00 mmol), and Cs₂CO₃ (782 mg, 2.40 mmol). DMF (20.0 mL) was added, and the reaction mixture was stirred at 80° C. for 18 hours. The crude reaction mixture was diluted with EtOAc (200 mL) and water (150 mL). The aqueous mixture was extracted with EtOAc (50 mL×2). The organic phases were combined, washed with 0.5 wt % LiCl (aq, 50 mL) and brine (aq, 50 mL×3). The organic solution was then dried (Na₂SO₄) and concentrated. Column chromatography (silica gel, MeOH in DCM, gradient 1 to 15%) yielded the desired product. MS: 464 [M+1]⁺.

### (R)-6-((6-(3-hydroxy-3-methylpyrrolidine-1-carbonyl)-5-methylpyrrolo[1,2-b]pyridazin-4-yl)oxy)-2-methylbenzofuran-3-carboxylic acid (I-76)

To a solution of methyl (R)-6-((6-(3-hydroxy-3-methylpyrrolidine-1-carbonyl)-5-methylpyrrolo[1,2-b]pyridazin-4-yl)oxy)-2-methylbenzofuran-3-carboxylate (510 mg, 1.10 mmol) in THF (2.75 mL) and MeOH (2.75 mL) was added 1 M NaOH (aq, 3.30 mL, 3.30 mmol). The reaction mixture was stirred at 50° C. for 4 hours. The reaction mixture was diluted with water (20 mL), and the organic solvent was removed under reduced pressure. The aqueous solution was cooled in an ice bath and acidified with 1 M HCl until pH of 5. The product was isolated by filtration, washed with cold water, air dried and further dried under high vacuum. MS: 450 [M+1]⁺. ¹H NMR (500 MHz, DMSO-d₆) δ 8.07-7.98 (m, 2H), 7.92 (d, J=5.4 Hz, 1H), 7.71 (d, J=2.1 Hz, 1H), 7.30 (dd, J=8.5, 2.1 Hz, 1H), 5.65 (d, J=5.4 Hz, 1H), 4.86-4.77 (m, 1H), 3.76-3.43 (m, 4H), 2.76 (s, 3H), 2.56 (s, 3H), 1.88-1.78 (m, 2H), 1.36-1.25 (m, 3H).

TABLE 8
Using methods similar to that outlined in the synthesis of I-76, and the appropriate
starting materials, the following intermediates were prepared:

| Compound | | | Exact Mass |
|---|---|---|---|
| Number | Structure | Name | [M + 1]⁺ |
| I-77 | 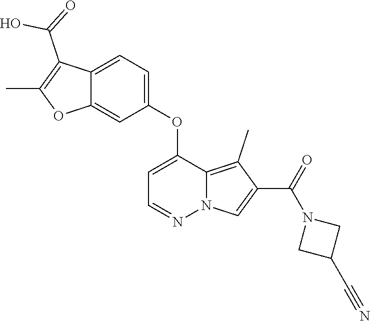 [SMILES: Cc1oc2cc(Oc3ccnn4cc(C(=O)N5CC(C#N)C5)c(C)c34)ccc2c1C(=O)O] | 6-((6-(3-cyanoazetidine- 1-carbonyl)-5- methylpyrrolo[1,2- b]pyridazin-4-yl)oxy)-2- methylbenzofuran-3- carboxylic acid | 431 |
| I-78 | 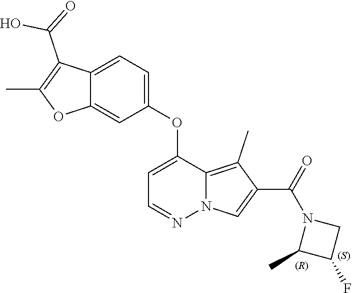 | 6-((6-((2R,3S)-3-fluoro- 2-methylazetidine-1- carbonyl)-5- methylpyrrolo[1,2- b]pyridazin-4-yl)oxy)-2- methylbenzofuran-3- carboxylic acid | 438 |

### Preparation of intermediate (2R,3R)-3-methoxy-2-methylazetidine, TFA (I-80)

Intermediate I-80 was prepared according to synthetic route outlined in Scheme 18.

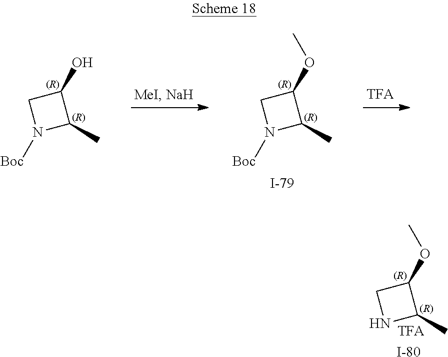

### tert-butyl (2R,3R)-3-methoxy-2-methylazetidine-1-carboxylate (I-79)

To a solution of tert-butyl (2R,3R)-3-hydroxy-2-methylazetidine-1-carboxylate (50.0 mg, 0.267 mmol) in THF (1.00 mL) was added NaH (60% in oil, 15.0 mg, 0.375 mmol) at 0° C. After stirring for 0.5 hours at 25° C., Mel (20.0 μL, 0.320 mmol) in THF (0.200 mL) was added to the mixture at 0° C. The resulting mixture was stirred for another 1 hour at 25° C. The reaction mixture was quenched with H₂O (0.5 mL) and extracted with EtOAc (1 mL×3). The combined organic phases were washed with brine (0.5 mL), dried over anhydrous Na₂SO₄, filtered and concentrated under reduced pressure to yield the desired product, which was used in the next step directly without further purification. ¹H NMR (400 MHz, CD₃OD) δ 4.44-4.35 (m, 1H), 4.16-4.09 (m, 1H), 4.05-3.98 (m, 1H), 3.71-3.62 (m, 1H), 3.29 (s, 3H), 1.43 (s, 9H), 1.31 (d, J=6.8 Hz, 3H).

### (2R,3R)-3-methoxy-2-methylazetidine, TFA (I-80)

A mixture of tert-butyl (2R,3R)-3-methoxy-2-methylazetidine-1-carboxylate (50 mg, 0.248 mmol) and TFA (0.300 mL, 3.89 mmol) in DCM (1 mL) was stirred at 25° C. for 2 hours. The mixture was concentrated under reduced pressure to yield the desired product. ¹H NMR (400 MHz, CD₃OD) δ 4.73-4.63 (m, 1H), 4.30-4.16 (m, 2H), 3.84-3.77 (m, 1H), 3.36 (s, 3H), 1.48 (d, J=7.2 Hz, 3H).

### Preparation of intermediate 2,3-dimethylazetidin-3-ol, hydrochloride (I-82)

Intermediate I-82 was prepared according to synthetic route outlined in Scheme 19.

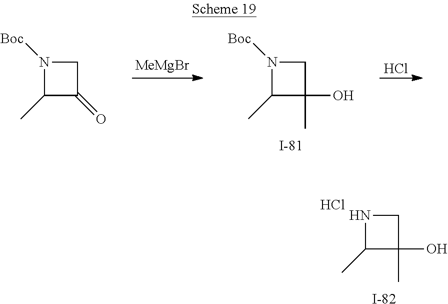

### tert-butyl 3-hydroxy-2,3-dimethylazetidine-1-carboxylate (I-81)

To an ice-cooled solution of tert-butyl 2-methyl-3-oxoazetidine-1-carboxylate (250 mg, 1.40 mmol) in THF (10.0 mL) was added dropwise a solution of magnesium methylbromide (483 mg, 1.20 mL, 3.4 M, 4.10 mmol) in 2-MeTHF. The resulting reaction mixture was stirred at 0° C. for 3 hours and then warmed to ambient temperature for 16 hours. The reaction mixture was quenched with saturated NH₄Cl (aq) (10 mL), and the mixture was extracted with EtOAc (2×10 mL). The combined organic fractions were dried (MgSO₄), and filtered. The solvent was evaporated under reduced pressure to afford the desired product, which was used in the next step directly without further purification. ¹H NMR (600 MHz, CDCl₃) δ 4.04 (q, J=6.5 Hz, 1H), 3.82 (d, J=9.3 Hz, 1H), 3.76 (d, J=9.3 Hz, 1H), 1.47 (s, 3H), 1.47 (s, 10H), 1.34 (d, J=6.5 Hz, 3H).

### 2,3-dimethylazetidin-3-ol, hydrochloride (I-82)

To a stirring solution of tert-butyl 3-hydroxy-2,3-dimethylazetidine-1-carboxylate (244 mg, 1.20 mmol) in 1,4-dioxane (4.00 mL) and water (1.00 mL) was added hydrochloric acid (619 mg, 4.20 mL, 4.0 M, 17.0 mmol) and the resulting reaction mixture was stirred at 25° C. for 16 hours. The reaction mixture was concentrated and lyophilized overnight to afford the desired product. ¹H NMR (600 MHz, CD₃OD) δ 4.30 (q, J=6.9 Hz, 1H), 3.93 (d, J=10.9 Hz, 1H), 3.77-3.73 (m, 1H), 1.48 (s, 3H), 1.45 (d, J=6.9 Hz, 3H).

### Preparation of intermediate (2R,3R)-2,3-dimethylazetidin-3-ol (I-7)

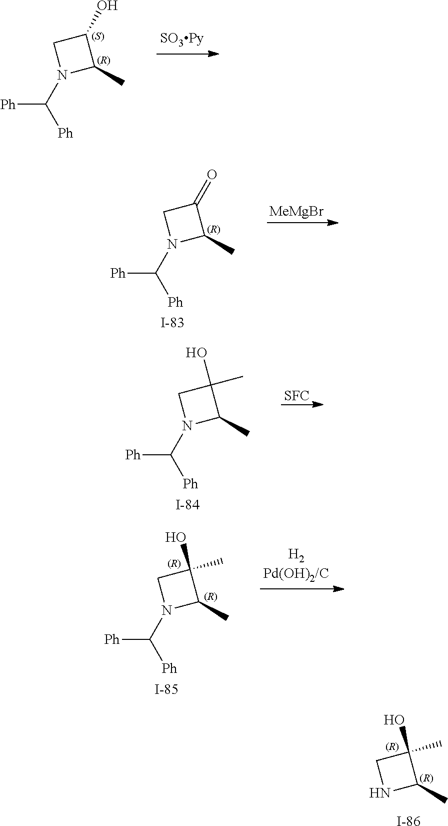

### (R)-1-benzhydryl-2-methylazetidin-3-one (I-4)

To a solution of pyridine-sulfur trioxide (94.2 g, 592 mmol) in DMSO (300 mL) and DCM (150 mL) was added dropwise (2R,3S)-1-benzhydryl-2-methyl-azetidin-3-ol (50 g, 197 mmol) and TEA (60 g, 592 mmol, 82 mL) in DMSO (300 mL) and DCM (150 mL) at 0° C. The resulting mixture was stirred at 0° C. for 2 hrs. The mixture was addition H₂O (500 mL) and extracted with EtOAc (1 L×3). The combined organic layers were washed with brine (500 mL), dried over Na₂SO₄, concentrated under reduced pressure to give the crude product which was carried forward without additional manipulation.

### (2R)-1-benzhydryl-2,3-dimethylazetidin-3-ol (I-5)

To a solution of (2R)-1-benzhydryl-2-methyl-azetidin-3-one I-4 (163 g, 648 mmol) in THF (1.63 L) was added MeMgBr (3 M, 648 mL). The mixture was stirred at −78° C. for 0.5 hr under N₂ atmosphere. The reaction mixture was quenched with saturated NH₄Cl (aq, 1 L), the mixture was extracted with EtOAc (1 L×3). The combined organic layers were washed with brine (500 mL), dried over Na₂SO₄, filtered and concentrated under reduced pressure. The residue was purified by column chromatography over silica gel (0-50% EtOAc in hexanes) to afford the desired product.

### (2R,3R)-1-benzhydryl-2,3-dimethylazetidin-3-ol (I-6)

Diastereomeric mixture I-5 was further separated by SFC (condition: column: DAICEL CHIRALCEL OJ (250 mm×50 mm, 10 m); mobile phase: [CO₂-EtOH (0.1% NH₃·H₂O)]; B %:30%, isocratic elution mode), to afford the title compound as the major isomer.

### (2R,3R)-2,3-dimethylazetidin-3-ol (I-7)

To a solution of I-6 (22 g, 82 mmol) in MeOH (440 mL) and HCl (1 M, 82 mL) was added Pd(OH)₂/C (10%, 4 g) under N₂ atmosphere. The suspension was degassed and purged with H₂ for 3 times. The mixture was stirred under H₂ (15 Psi) at 20° C. for 12 hrs. The mixture was filtered and concentrated under reduced pressure to give a residue. To the mixture was added H₂O (50 mL) and it was then extracted with hexane (500 mL×3). The combined organic layers were washed with brine (200 mL), dried over Na₂SO₄, and concentrated under reduced pressure to give the title compound. MS: 102 [M+1]⁺. ¹H NMR (400 MHz, CD₃OD) δ: 4.40-4.22 (m, 1H), 3.92 (d, J=10.8 Hz, 1H), 3.75 (d, J=10.8 Hz, 1H), 1.52-1.35 (m, 6H).

### Preparation of intermediate (2S,3S)-2,3-dimethylazetidin-3-ol (I-8)

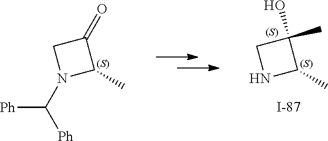

The title compound was prepared utilizing an identical sequence to that used for the preparation of I-7, but proceeding via the enantiomer of I-4. MS: 102 [M+1]⁺. ¹H NMR (400 MHz, CD₃OD) δ: 4.40-4.22 (m, 1H), 3.92 (d, J=10.8 Hz, 1H), 3.75 (d, J=10.8 Hz, 1H), 1.52-1.35 (m, 6H).

### EXAMPLES

### Preparation of Example 1.1: methyl 4-((2-methyl-3-(methylcarbamoyl)benzofuran-6-yl)oxy)pyrrolo[1,2-b]pyridazine-6-carboxylate

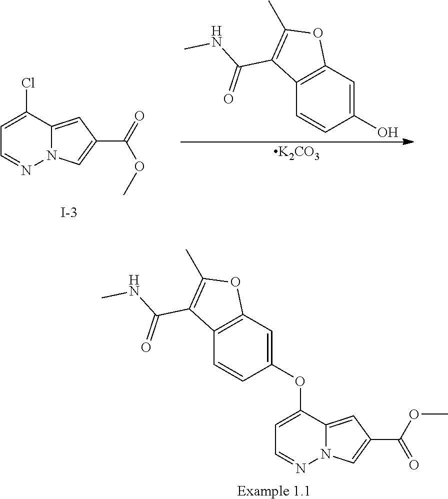

To a solution of 6-hydroxy-N,2-dimethylbenzofuran-3-carboxamide (9.74 mg, 50.0 μmol) in DMF (0.500 mL) was added K₂CO₃ (19.7 mg, 0.140 mmol), and the mixture was stirred at 25° C. for 5 minutes. Methyl 4-chloropyrrolo[1,2-b]pyridazine-6-carboxylate (10 mg, 0.047 mmol) was then added and the resulting reaction mixture was stirred at 60° C. for 2 hours. The reaction was purified by mass-directed reverse phase HPLC (MeCN/water with 0.1% TFA modifier) to afford the desired product. MS: 380 [M+1]⁺. ¹H NMR (400 MHz, CD₃OD) δ 8.21 (d, J=1.6 Hz, 1H), 7.99 (d, J=5.6 Hz, 1H), 7.83 (d, J=8.8 Hz, 1H), 7.45 (d, J=2.0 Hz, 1H), 7.21 (d, J=8.4 Hz, 1H), 7.10 (d, J=1.6 Hz, 1H), 5.83 (d, J=5.6 Hz, 1H), 3.90 (s, 3H), 2.97 (s, 3H), 2.67 (s, 3H).

TABLE 9
Utilizing a method similar to that described for the preparation of Example 1.1 and the
appropriate starting materials, the following examples were prepared:

| Compound | | | Exact Mass |
|---|---|---|---|
| Number | Structure | Name | [M + 1]⁺. |
| Example 1.2 | 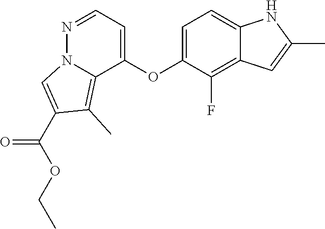 [SMILES: CCOC(=O)c1cn2nccc(Oc3ccc4[nH]c(C)cc4c3F)c2c1C] | ethyl 4-[(4-fluoro- 2-methyl-1H- indol-5-yl)oxy]-5- methylpyrrolo[1,2-b] pyridazine-6-carboxylate | 368 |

### 1Preparation of Example 2.1: ethyl 5-methyl-4-{[2-methyl-3-(methylcarbamoyl)-1-benzofuran-6-yl]oxy}pyrrolo[1,2-b]pyridazine-6-carboxylate

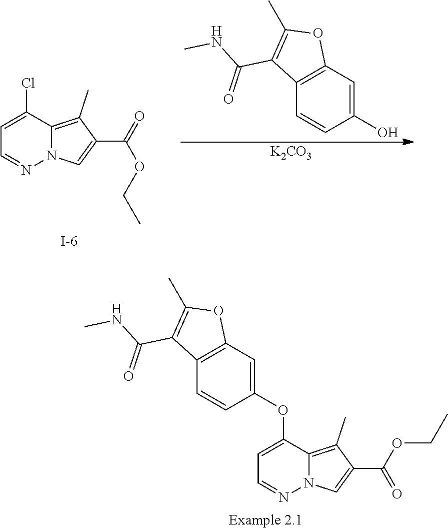

To a 100 mL flask, ethyl 4-chloro-5-methylpyrrolo[1,2-b]pyridazine-6-carboxylate (5.00 g, 20.9 mmol), 6-hydroxy-N,2-dimethylbenzofuran-3-carboxamide (4.51 g, 22.0 mmol) and potassium carbonate (5.79 g, 41.9 mmol) were added. NMP (52.4 mL) was added to the reaction vessel and the resulting mixture was stirred at 80° C. for 16 hours. The product mixture was cooled down to room temperature and then triturated with water (150 mL) and filtered. The solid was dried overnight with a positive flow of air to afford the desired product. MS: 408 [M+H]⁺. ¹H NMR (400 MHz, CD₃OD) δ 8.12 (s, 1H), 7.86-7.82 (m, 2H), 7.45 (d, J=2.0 Hz, 1H), 7.23-7.19 (m, 1H), 5.63 (d, J=5.6 Hz, 1H), 4.35 (q, J=7.2 Hz, 2H), 2.98 (s, 3H), 2.80 (s, 3H), 2.67 (s, 3H), 1.40 (t, J=6.8 Hz, 3H).

### Preparation of Example 3.1: 26-((6-((2R,3R)-3-hydroxy-2-methylazetidine-1-carbonyl)-5-methylpyrrolo[1,2-b]pyridazin-4-yl)oxy)-N,2-dimethylbenzofuran-3-carboxamide

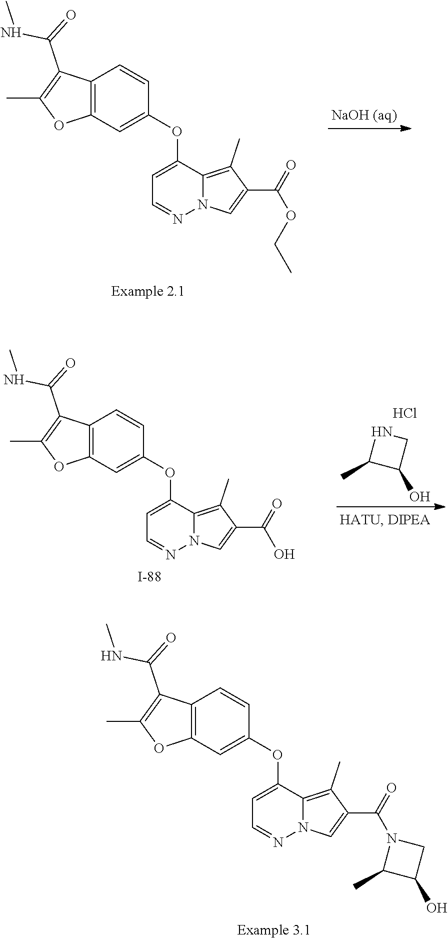

### 5-methyl-4-((2-methyl-3-(methylcarbamoyl)benzofuran-6-yl)oxy)pyrrolo[1,2-b]pyridazine-6-carboxylic acid (I-88)

To a solution of ethyl 5-methyl-4-((2-methyl-3-(methylcarbamoyl)benzofuran-6-yl)oxy)pyrrolo[1,2-b]pyridazine-6-carboxylate (8.30 g, 20.4 mmol) in THF (102 mL) and ethanol (102 mL), 2 M sodium hydroxide (aq, 2.44 g, 30.6 mL, 61.1 mmol) was added. The resulting mixture was stirred at 50° C. for 16 hours. The product mixture was concentrated under reduced pressure and was resuspended in water (100 mL). After the mixture was cooled to 0° C., 6 M HCl (aq) was added until pH˜5, and the resulting mixture was stirred for 15 min. The mixture was further acidified to pH˜1 and the solid formed was collected through filtration and was dried overnight with a positive flow of air to afford the desired product. MS: 380 [M+H]⁺.

### 6-((6-((2R,3R)-3-hydroxy-2-methylazetidine-1-carbonyl)-5-methylpyrrolo[1,2-b]pyridazin-4-yl)oxy)-N,2-dimethylbenzofuran-3-carboxamide (Example 3.1)

Four 20 mL vials, each equipped with a magnetic stirrer, were each charged with 5-methyl-4-((2-methyl-3-(methylcarbamoyl)benzofuran-6-yl)oxy)pyrrolo[1,2-b]pyridazine-6-carboxylic acid (120 mg, 0.320 mmol), (2R,3R)-2-methylazetidin-3-ol hydrochloride (117 mg, 0.950 mmol), and HATU (360 mg, 0.950 mmol). To the vials under nitrogen atmosphere were added DMF (4.00 mL), followed by DIPEA (385 μL, 2.20 mmol), and the mixtures were stirred at room temperature overnight. The mixtures were filtered, and the filtrates were subjected to purification by preparative reverse phase HPLC (H₂O/MeCN/TFA). The returned fractions were subjected to liquid-liquid extraction, partitioning between saturated aqueous NaHCO₃ (150 mL) and a 3:1 v/v mixture of CHCl₃/IPA (25 mL×3). The combined organic layers were dried over anhydrous sodium sulfate, filtered, and the collected filtrate was concentrated under reduced pressure. A 5 mg aliquot of this material was then suspended in MTBE (100 μL) and stirred vigorously overnight. The bulk residue was then co-evaporated from ethanol (50 mL). Co-evaporation from ethanol was then repeated twice more. The resulting solid was dissolved in ethanol (2 mL), in a 40 mL vial. The mixture was warmed to 40° C., at which point MTBE was added slowly until a cloudy mixture was formed (2 mL). At this point, the 5 mg suspension of compound in MTBE was seeded into the stirring mixture by pipette, and stirring was continued at 40° C. for 4 hours. Upon cooling to room temperature, additional MTBE was added (5 mL), and the mixture was then stirred at room temperature for 72 hours under nitrogen. Solids were collected by vacuum filtration and washed with a minimal amount of cold MTBE. The obtained crystalline material was then dried in the vacuum oven at 40° C. for 18 hours. MS: 449 [M+H]⁺. ¹H NMR (500 MHz, DMSO-d₆) δ: 8.01 (s, 1H), 7.97 (q, J=4.4 Hz, 1H), 7.93 (d, J=5.4 Hz, 1H), 7.85 (d, J=8.5 Hz, 1H), 7.66 (s, 1H), 7.31-7.22 (m, 1H), 5.61 (d, J=5.4 Hz, 1H), 5.50 (d, J=5.7 Hz, 1H), 4.65 (br m, 2H), 4.42-3.64 (br m, 3H), 2.82 (d, J=4.6 Hz, 3H), 2.64 (d, J=4.9 Hz, 6H), 1.33 (br m, 2H), 1.05 (br m, 1H).

### Preparation of Example 4.1: 3(R)-6-((6-(3-hydroxy-3-methylpyrrolidine-1-carbonyl)-5-methylpyrrolo[1,2-b]pyridazin-4-yl)oxy)-N,2-dimethylbenzofuran-3-carboxamide

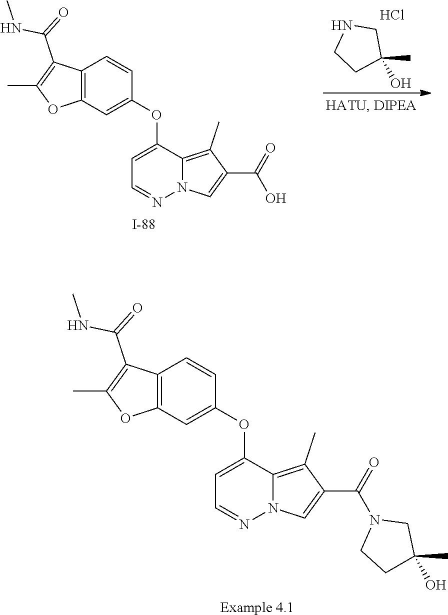

5-methyl-4-((2-methyl-3-(methylcarbamoyl)benzofuran-6-yl)oxy)pyrrolo[1,2-b]pyridazine-6-carboxylic acid (7.60 g, 20.0 mmol) and (R)-3-methylpyrrolidin-3-ol, HCl (2.89 g, 21.0 mmol) were added into a 250 mL round bottom flask. MeCN (61.0 mL) was added into the reaction vessel. DIPEA (8.72 mL, 50.0 mmol) was added into the reaction vessel followed by addition of HATU (11.4 g, 30.0 mmol) in a single portion. DMF (6.10 mL) was added into the reaction vessel, and the resulting mixture was stirred for 2 hours. The product mixture was concentrated on the Rotovap. The residue obtained was diluted with MEK (200 mL). The diluted product mixture was washed with brine (100 mL×3). The aqueous layer was then back extracted with MEK (100 mL). The combined MEK layer was then washed again with brine (50 mL×3). The combined organic layers were dried over anhydrous sodium sulfate, filtered, and the collected filtrate was concentrated under reduced pressure. The resulting crude residue was purified by flash column chromatography (120 g, amino functionalized silica gel, 0 to 60% of 3:1 v/v EtOAc:ethanol in hexanes) to provide the desired product. The product was then co-evaporated with isopropyl acetate (50 mL). Co-evaporation from IPAc was then repeated twice more. The amorphous material was transferred to a 100 mL EasyMax™ reactor (Mettler Toledo; Columbus, OH). Isopropyl acetate (70 mL) was added into the reaction vessel. The resulting mixture was heated at 45° C. for 2 hours. Ethanol (7 mL) was then added dropwise into the reaction vessel. The resulting mixture was stirred at 45° C. for 30 min. The supernatant was sampled (1 mL) and seeded with crystalline material (5 mg). The resulting aliquot was stirred for 30 minutes at room temperature. The seeded aliquot was then transferred into the reactor via pipette. The resulting mixture was held at 45° C. for 6 hours, then cooled down to 15° C. over 6 hours. Solids were then collected by vacuum filtration. The obtained crystalline product was then dried with full house vacuum applied under a positive pressure of nitrogen for 48 hours. MS: 463 [M+H]⁺. ¹H NMR (500 MHz, DMSO-d₆) δ: 8.03 (m, 1H), 7.97 (d, J=4.6 Hz, 1H), 7.92 (d, J=5.4 Hz, 1H), 7.85 (d, J=8.5 Hz, 1H), 7.66 (d, J=2.1 Hz, 1H), 7.26 (dd, J=8.5, 2.1 Hz, 1H), 5.62 (d, J=5.4 Hz, 1H), 4.83 (s, 1H, minor rotamer), 4.76 (s, 1H, major rotamer), 3.75-3.59 (m, 1H), 3.55 (m, 1H), 3.47 (dd, J=11.5, 6.1 Hz, 1H), 3.35 (d, J=8.2 Hz, 1H), 2.83 (d, J=4.6 Hz, 3H), 2.64 (s, 3H), 2.55 (s, 3H), 1.91-1.75 (m, 2H), 1.34 (s, 3H, minor rotamer), 1.26 (s, 3H, major rotamer).

### 4Preparation of Example 5.1: N,2-dimethyl-6-((6-(pyrrolidine-1-carbonyl)pyrrolo[1,2-b]pyridazin-4-yl)oxy)benzofuran-3-carboxamide

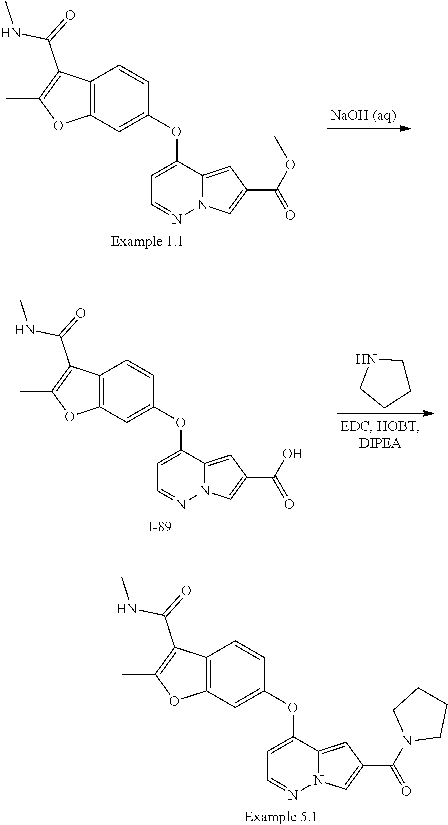

### 4-((2-methyl-3-(methylcarbamoyl)benzofuran-6-yl)oxy)pyrrolo[1,2-b]pyridazine-6-carboxylic acid (I-89)

To a solution of methyl 4-((2-methyl-3-(methylcarbamoyl)benzofuran-6-yl)oxy)pyrrolo[1,2-b]pyridazine-6-carboxylate (150 mg, 0.400 mmol) in ethanol/water (4:1) (2.00 mL) was added NaOH (47.4 mg, 1.19 mmol), and the resulting reaction mixture was stirred at 60° C. under N₂ for 2 hours. The reaction mixture was acidified with 1 M HCl (aq.) to pH 5, and the mixture was then dried under vacuum and was used directly in the next step without further purification. MS: 366 [M+1]⁺.

### N,2-dimethyl-6-((6-(pyrrolidine-1-carbonyl)pyrrolo[1,2-b]pyridazin-4-yl)oxy)benzofuran-3-carboxamide (Example 5.1)

To a solution of 4-((2-methyl-3-(methylcarbamoyl)benzofuran-6-yl)oxy)pyrrolo[1,2-b]pyridazine-6-carboxylic acid (144 mg, 0.400 mmol) in DMF (1.50 mL) was added EDC (113 mg, 0.590 mmol), 1H-benzo[d][1,2,3]triazol-1-ol (133 mg, 0.990 mmol), DIPEA (0.170 mL, 0.990 mmol) and pyrrolidine (30.8 mg, 0.430 mmol). The resulting reaction mixture was stirred at 25° C. for 36 hours. The mixture was purified by prep-TLC (SiO₂, petroleum ether:EtOAc=1:2) to afford the desired product. MS: 419 [M+1]⁺. ¹H NMR (400 MHz, CD₃OD) δ 8.21 (d, J=1.6 Hz, 1H), 7.99 (d, J=5.6 Hz, 1H), 7.83 (d, J=8.4 Hz, 1H), 7.45 (d, J=2.0 Hz, 1H), 7.21 (d, 1H), 7.10 (d, J=1.6 Hz, 1H), 5.83 (d, J=5.6 Hz, 1H), 3.90 (s, 3H), 2.97 (s, 3H), 2.67 (s, 3H).

### Preparation of Example 6.1: 5N,2-dimethyl-6-((5-methyl-6-(pyrrolidine-1-carbonyl)pyrrolo[1,2-b]pyridazin-4-yl)oxy)benzofuran-3-carboxamide

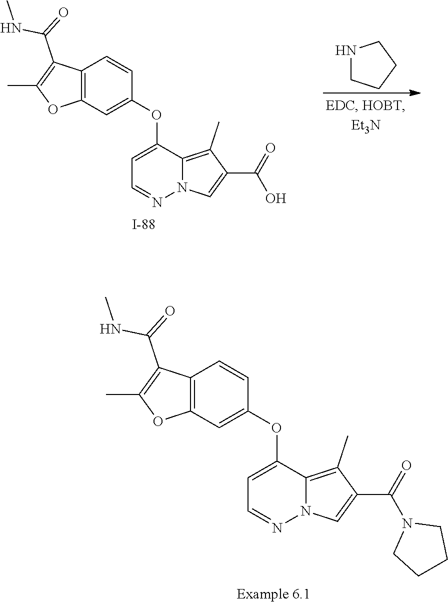

A mixture of 5-methyl-4-((2-methyl-3-(methylcarbamoyl)benzofuran-6-yl)oxy)pyrrolo[1,2-b]pyridazine-6-carboxylic acid (50.0 mg, 0.130 mmol), pyrrolidine (20.0 μL, 0.200 mmol), HOBT (60.6 mg, 0.400 mmol), EDC (76.0 mg, 0.400 mmol), TEA (90.0 μL, 0.660 mmol) in DMF (2.00 mL) was stirred at 25° C. for 2 hours. The mixture was filtered, and the filtrate was purified by mass-directed reverse phase HPLC (MeCN/water with 0.1% TFA modifier) to afford the desired product. MS: 433 [M+1]⁺. ¹H NMR (400 MHz, CD₃OD) δ 7.89 (s, 1H), 7.85-7.81 (m, 2H), 7.43 (d, J=2.0 Hz, 1H), 7.23-7.18 (m, 1H), 5.65 (d, J=5.2 Hz, 1H), 3.63 (t, J=6.8 Hz, 2H), 3.56 (t, J=6.4 Hz, 2H), 2.98 (s, 3H), 2.67 (s, 3H), 2.61 (s, 3H), 2.05-1.94 (m, 4H).

TABLE 10
Utilizing a method similar to that described for the preparation of Example 6.1 and
the appropriate starting materials, the following examples were prepared:

| Compound | | | Exact Mass |
|---|---|---|---|
| Number | Structure | Name | [M + 1]⁺ |
| Example 6.2 | 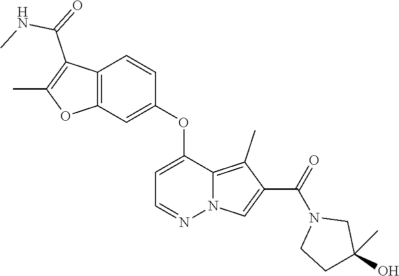 [SMILES: CNC(=O)c1c(C)oc2cc(Oc3ccnn4cc(C(=O)N5CC[C@](C)(O)C5)c(C)c34)ccc12] | 6-({6-[(3S)-3-hydroxy-3- methylpyrrolidine-1-carbonyl]- 5-methylpyrrolo[1,2- b]pyridazin-4-yl}oxy)-N,2- dimethyl-1-benzofuran-3- carboxamide | 463 |
| Example 6.3 | 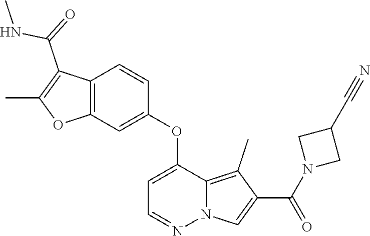 [SMILES: CNC(=O)c1c(C)oc2cc(Oc3ccnn4cc(C(=O)N5CC(C#N)C5)c(C)c34)ccc12] | 6-{[6-(3-cyanoazetidine-1- carbonyl)-5- methylpyrrolo[1,2-b]pyridazin- 4-yl]oxy}-N,2-dimethyl-1- benzofuran-3-carboxamide | 444 |
| Example 6.4 | 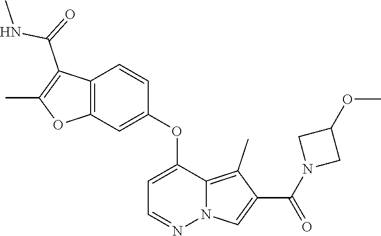 | 6-{[6-(3-methoxyazetidine-1- carbonyl)-5- methylpyrrolo[1,2-b]pyridazin- 4-yl]oxy}-N,2-dimethyl-1- benzofuran-3-carboxamide | 449 |
| Example 6.5 | 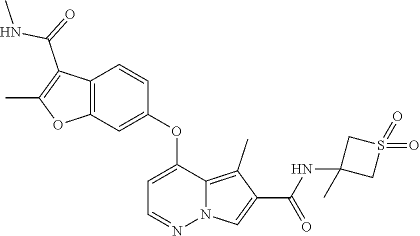 [SMILES: CNC(=O)c1c(C)oc2cc(Oc3ccnn4cc(C(=O)NC5(C)CS(=O)(=O)C5)c(C)c34)ccc12] | 5-methyl-N-(3-methyl-1,1- dioxo-1lambda~6~-thietan-3- yl)-4-{[2-methyl-3- (methylcarbamoyl)-1- benzofuran-6- yl]oxy}pyrrolo[1,2- b]pyridazine-6-carboxamide | 497 |
| Example 6.6 | 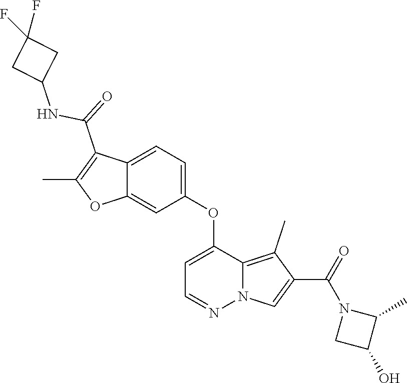 [SMILES: Cc1oc2cc(Oc3ccnn4cc(C(=O)N5C[C@@H](O)[C@H]5C)c(C)c34)ccc2c1C(=O)NC1CC(F)(F)C1] | N-(3,3-difluorocyclobutyl)-6- ({6-[(2R,3R)-3-hydroxy-2- methylazetidine-1-carbonyl]-5- methylpyrrolo[1,2-b]pyridazin- 4-yl}oxy)-2-methyl-1- benzofuran-3-carboxamide | 525 |
| Example 6.7 | 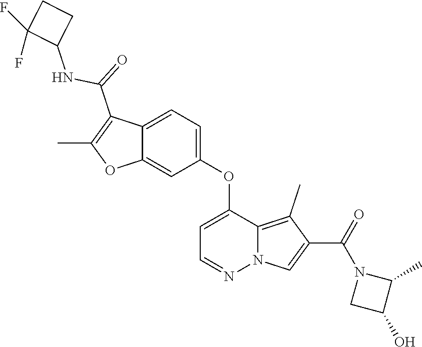 [SMILES: Cc1oc2cc(Oc3ccnn4cc(C(=O)N5C[C@@H](O)[C@H]5C)c(C)c34)ccc2c1C(=O)NC1CCC1(F)F] | N-({R or S}-2,2- difluorocyclobutyl)-6-({6- [(2R,3R)-3-hydroxy-2- methylazetidine-1-carbonyl]-5- methylpyrrolo[1,2-b]pyridazin- 4-yl}oxy)-2-methyl-1- benzofuran-3-carboxamide | 525 |
| Example 6.8 | 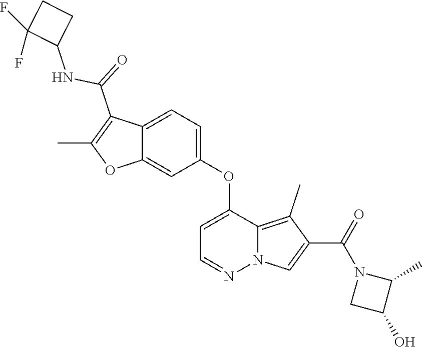 [SMILES: Cc1oc2cc(Oc3ccnn4cc(C(=O)N5C[C@@H](O)[C@H]5C)c(C)c34)ccc2c1C(=O)NC1CCC1(F)F] | N-({S or R}-2,2- difluorocyclobutyl)-6-({6- [(2R,3R)-3-hydroxy-2- methylazetidine-1-carbonyl]-5- methylpyrrolo[1,2-b]pyridazin- 4-yl}oxy)-2-methyl-1- benzofuran-3-carboxamide | 525 |
| Example 6.9 | 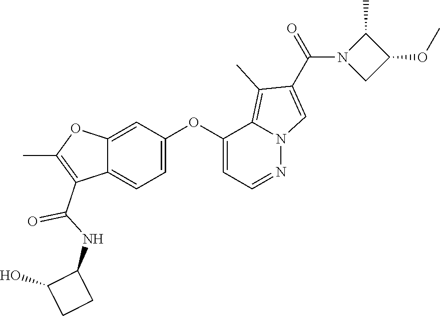 [SMILES: CO[C@@H]1CN(C(=O)c2cn3nccc(Oc4ccc5c(C(=O)N[C@H]6CC[C@@H]6O)c(C)oc5c4)c3c2C)[C@@H]1C] | N-[(1S,2S)-2- hydroxycyclobutyl]-6-({6- [(2R,3R)-3-methoxy-2- methylazetidine-1-carbonyl]-5- methylpyrrolo[1,2-b]pyridazin- 4-yl}oxy)-2-methyl-1- benzofuran-3-carboxamide | 519 |
| Example 6.10 | 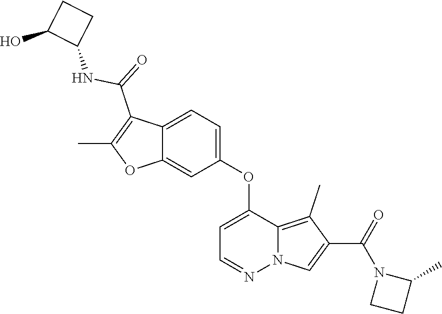 [SMILES: Cc1oc2cc(Oc3ccnn4cc(C(=O)N5CC[C@H]5C)c(C)c34)ccc2c1C(=O)N[C@H]1CC[C@@H]1O] | N-[(1S,2S)-2- hydroxycyclobutyl]-2-methyl- 6-({5-methyl-6-[(2R)-2- methylazetidine-1- carbonyl]pyrrolo[1,2- b]pyridazin-4-yl}oxy)-1- benzofuran-3-carboxamide | 489 |
| Example 6.11 | 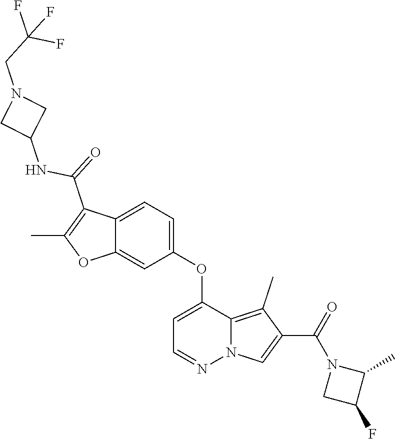 [SMILES: Cc1oc2cc(Oc3ccnn4cc(C(=O)N5C[C@H](F)[C@H]5C)c(C)c34)ccc2c1C(=O)NC1CN(CC(F)(F)F)C1] | 6-9{(6-[(2R,3S)-3-fluoro-2- methylazetidine-1-carbonyl]-5- methylpyrrolo[1,2-b]pyridazin- 4-yl}oxy)-2-methyl-N-[1- (2,2,2-trifluoroethyl)azetidin- 3-yl]-1-benzofuran-3- carboxamide | 574 |
| Example 6.12 | 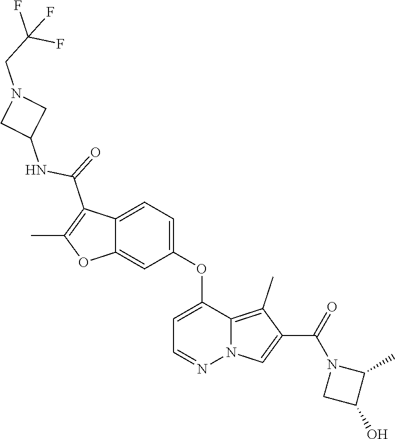 [SMILES: Cc1oc2cc(Oc3ccnn4cc(C(=O)N5C[C@@H](O)[C@H]5C)c(C)c34)ccc2c1C(=O)NC1CN(CC(F)(F)F)C1] | 6-({6-[(2R,3R)-3-hydroxy-2- methylazetidine-1-carbonyl]-5- methylpyrrolo[1,2-b]pyridazin- 4-yl}oxy)-2-methyl-N-[1- (2,2,2-trifluoroethyl)azetidin- 3-yl]-1-benzofuran-3-carboxamide | 572 |
| Example 6.13 | 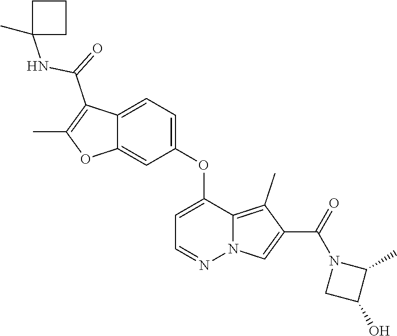 | 6-({6-[(2R,3R)-3-hydroxy-2- methylazetidine-1-carbonyl]-5- methylpyrrolo[1,2-b]pyridazin- 4-yl}oxy)-2-methyl-N-(1- methylcyclobutyl)-1- benzofuran-3-carboxamide | 503 |
| Example 6.14 | 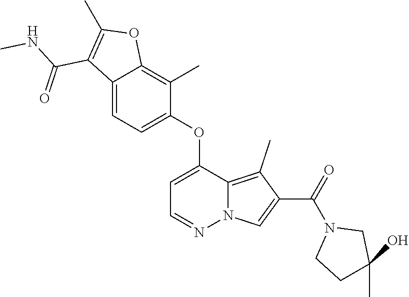 [SMILES: CNC(=O)c1c(C)oc2c(C)c(Oc3ccnn4cc(C(=O)N5CC[C@](C)(O)C5)c(C)c34)ccc12] | 6-({6-[(3S)-3-hydroxy-3- methylpyrrolidine-1-carbonyl]- 5-methylpyrrolo[1,2- b]pyridazin-4-yl}oxy)-N,2,7- trimethyl-1-benzofuran-3- carboxamide | 477 |
| Example 6.15 | 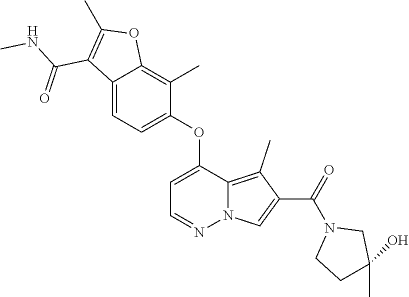 [SMILES: CNC(=O)c1c(C)oc2c(C)c(Oc3ccnn4cc(C(=O)N5CC[C@@](C)(O)C5)c(C)c34)ccc12] | 6-({6-[(3R)-3-hydroxy-3- methylpyrrolidine-1-carbonyl]- 5-methylpyrrolo[1,2- b]pyridazin-4-yl}oxy)-N,2,7- trimethyl-1-benzofuran-3- carboxamide | 477 |
| Example 6.16 | 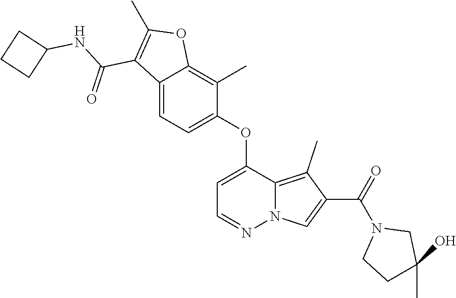 [SMILES: Cc1oc2c(C)c(Oc3ccnn4cc(C(=O)N5CC[C@](C)(O)C5)c(C)c34)ccc2c1C(=O)NC1CCC1] | N-cyclobutyl-6-({6-[(3S)-3- hydroxy-3-methylpyrrolidine- 1-carbonyl]-5- methylpyrrolo[1,2-b]pyridazin- 4-yl}oxy)-2,7-dimethyl-1- benzofuran-3-carboxamide | 517 |
| Example 6.17 | 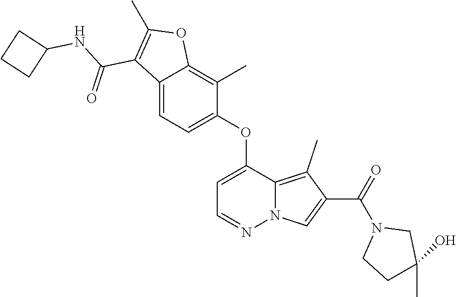 [SMILES: Cc1oc2c(C)c(Oc3ccnn4cc(C(=O)N5CC[C@@](C)(O)C5)c(C)c34)ccc2c1C(=O)NC1CCC1] | N-cyclobutyl-6-({6-[(3R)-3- hydroxy-3-methylpyrrolidine- 1-carbonyl]-5- methylpyrrolo[1,2-b]pyridazin- 4-yl}oxy)-2,7-dimethyl-1- benzofuran-3-carboxamide | 517 |
| Example 6.18 | 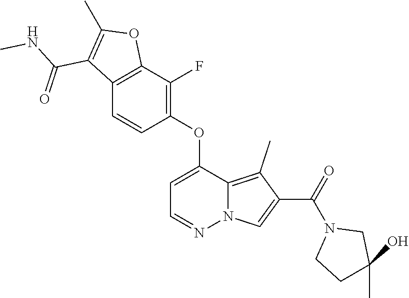 [SMILES: CNC(=O)c1c(C)oc2c(F)c(Oc3ccnn4cc(C(=O)N5CC[C@](C)(O)C5)c(C)c34)ccc12] | 7-fluoro-6-({6-[(3S)-3- hydroxy-3-methylpyrrolidine- 1-carbonyl]-5- methylpyrrolo[1,2-b]pyridazin- 4-yl}oxy)-N,2-dimethyl-1- benzofuran-3-carboxamide | 481 |
| Example 6.19 | 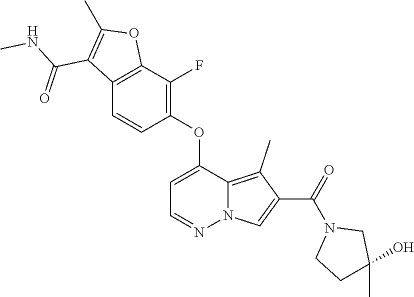 [SMILES: CNC(=O)c1c(C)oc2c(F)c(Oc3ccnn4cc(C(=O)N5CC[C@@](C)(O)C5)c(C)c34)ccc12] | 7-fluoro-6-({6-[(3R)-3- hydroxy-3-methylpyrrolidine- 1-carbonyl]-5- methylpyrrolo[1,2-b]pyridazin- 4-yl}oxy)-N,2-dimethyl-1- benzofuran-3-carboxamide | 481 |
| Example 6.20 | 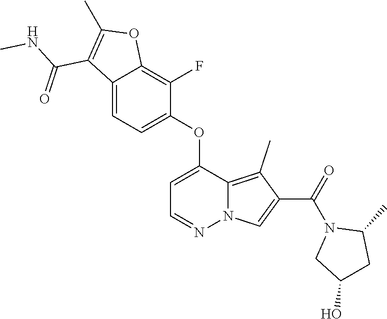 | 7-fluoro-6-({6-[(2R,4S)-4- hydroxy-2-methylpyrrolidine- 1-carbonyl]-5- methylpyrrolo[1,2-b]pyridazin- 4-yl}oxy)-N,2-dimethyl-1- benzofuran-3-carboxamide | 481 |
| Example 6.21 | 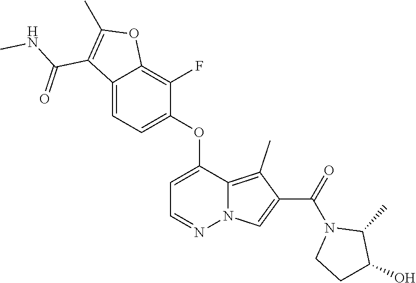 [SMILES: CNC(=O)c1c(C)oc2c(F)c(Oc3ccnn4cc(C(=O)N5CC[C@@H](O)[C@H]5C)c(C)c34)ccc12] | 7-fluoro-6-({6-[(2R,3R)-3- hydroxy-3-methylpyrrolidine- 1-carbonyl]-5- methylpyrrolo[1,2-b]pyridazin- 4-yl}oxy)-N,2-dimethyl-1- benzofuran-3-carboxamide | 481 |
| Example 6.22 | 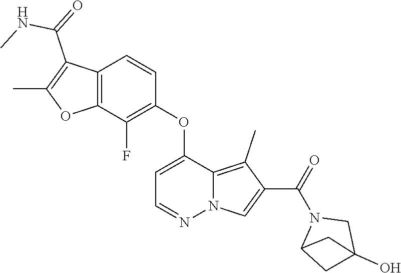 [SMILES: CNC(=O)c1c(C)oc2c(F)c(Oc3ccnn4cc(C(=O)N5CC6(O)CC5C6)c(C)c34)ccc12] | 7-fluoro-6-{[6-(4-hydroxy-2- azabicyclo[2.1.1]hexane-2- carbonyl)-5- methylpyrrolo[1,2-b]pyridazin- 4-yl]oxy}-N,2-dimethyl-1- benzofuran-3-carboxamide | 479 |
| Example 6.23 | 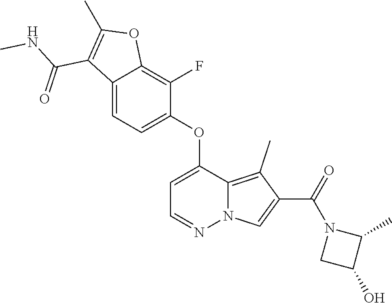 | 7-fluoro-6-({6-[(2R,3R)-3- hydroxy-2-methylazetidine-1- carbonyl]-5- methylpyrrolo[1,2-b]pyridazin- 4-yl}oxy)-N,2-dimethyl-1- benzofuran-3-carboxamide | 467 |
| Example 6.24 | 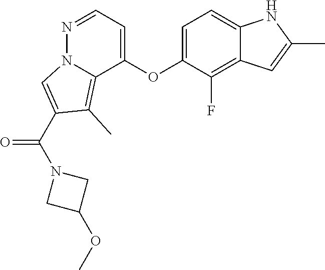 [SMILES: COC1CN(C(=O)c2cn3nccc(Oc4ccc5[nH]c(C)cc5c4F)c3c2C)C1] | {4-[(4-fluoro-2-methyl-1H- indol-5-yl)oxy]-5- methylpyrrolo[1,2-b]pyridazin- 6-yl}(3-methoxyazetidin-1- yl)methanone | 409 |
| Example 6.25 | 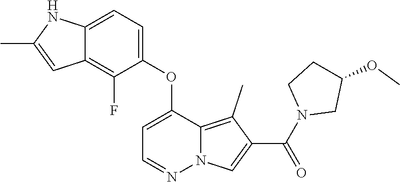 | {4-[(4-fluoro-2-methyl-1H- indol-5-yl)oxy]-5- methylpyrrolo[1,2-b]pyridazin- 6-yl}[(3S)-3- methoxypyrrolidin-1- yl]methanone | 423 |
| Example 6.26 | 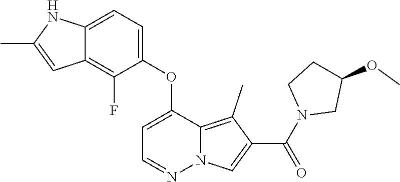 | {4-[(4-fluoro-2-methyl-1H- indol-5-yl)oxy]-5- methylpyrrolo[1,2-b]pyridazin- 6-yl}[(3R)-3- methoxypyrrolidin-1- yl]methanone | 423 |
| Example 6.27 | 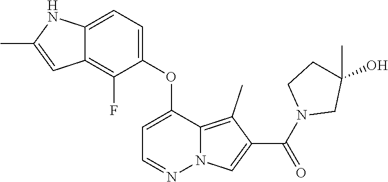 [SMILES: Cc1cc2c(F)c(Oc3ccnn4cc(C(=O)N5CC[C@](C)(O)C5)c(C)c34)ccc2[nH]1] | {4-[(4-fluoro-2-methyl-1H- indol-5-yl)oxy]-5- methylpyrrolo[1,2-b]pyridazin- 6-yl}[(3S)-3-hydroxy-3- methylpyrrolidin-1- yl]methanone | 423 |
| Example 6.28 | 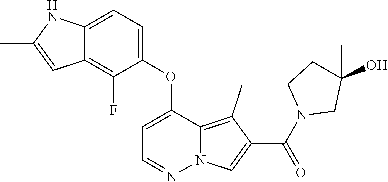 [SMILES: Cc1cc2c(F)c(Oc3ccnn4cc(C(=O)N5CC[C@@](C)(O)C5)c(C)c34)ccc2[nH]1] | {4-[(4-fluoro-2-methyl-1H- indol-5-yl)oxy]-5- methylpyrrolo[1,2-b]pyridazin- 6-yl}[(3R)-3-hydroxy-3- methylpyrrolidin-1- yl]methanone | 423 |
| Example 6.29 | 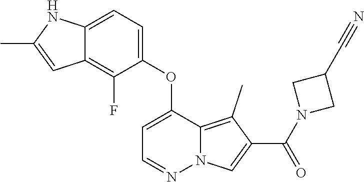 [SMILES: Cc1cc2c(F)c(Oc3ccnn4cc(C(=O)N5CC(C#N)C5)c(C)c34)ccc2[nH]1] | 1-{4-[(4-fluoro-2-methyl-1H- indol-5-yl)oxy]-5- methylpyrrolo[1,2- b]pyridazine-6- carbonyl}azetidine-3- carbonitrile | 404 |
| Example 6.30 | 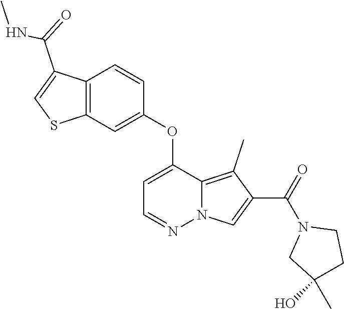 [SMILES: CNC(=O)c1csc2cc(Oc3ccnn4cc(C(=O)N5CC[C@](C)(O)C5)c(C)c34)ccc12] | 6-({6-[(3S)-3-hydroxy-3- methylpyrrolidine-1-carbonyl]- 5-methylpyrrolo[1,2- b]pyridazin-4-yl}oxy)-N- methyl-1-benzothiophene-3- carboxamide | 465 |
| Example 6.31 | 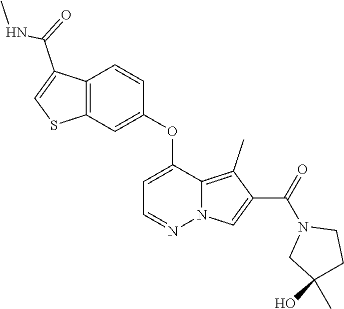 [SMILES: CNC(=O)c1csc2cc(Oc3ccnn4cc(C(=O)N5CC[C@@](C)(O)C5)c(C)c34)ccc12] | 6-({6-[(3R)-3-hydroxy-3- methylpyrrolidine-1-carbonyl]- 5-methylpyrrolo[1,2- b]pyridazin-4-yl}oxy)-N- methyl-1-benzothiophene-3- carboxamide | 465 |
| Example 6.32 | 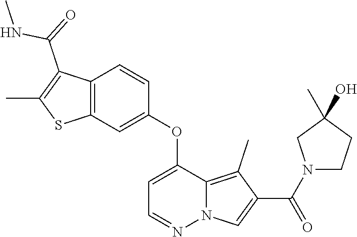 [SMILES: CNC(=O)c1c(C)sc2cc(Oc3ccnn4cc(C(=O)N5CC[C@](C)(O)C5)c(C)c34)ccc12] | 6-({6-[(3S)-3-hydroxy-3- methylpyrrolidine-1-carbonyl]- 5-methylpyrrolo[1,2- b]pyridazin-4-yl}oxy)-N,2- dimethyl-1-benzothiophene-3- carboxamide | 479 |
| Example 6.33 | 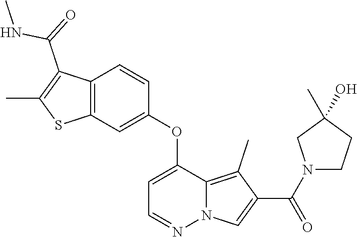 [SMILES: CNC(=O)c1c(C)sc2cc(Oc3ccnn4cc(C(=O)N5CC[C@@](C)(O)C5)c(C)c34)ccc12] | 6-({6-[(3R)-3-hydroxy-3- methylpyrrolidine-1-carbonyl]- 5-methylpyrrolo[1,2- b]pyridazin-4-yl}oxy)-N,2- dimethyl-1-benzothiophene-3- carboxamide | 479 |
| Example 6.34 | 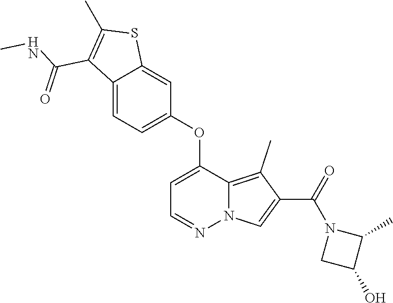 | 6-({6-[(2R,3R)-3-hydroxy-2- methylazetidine-1-carbonyl]-5- methylpyrrolo[1,2-b]pyridazin- 4-yl}oxy)-N,2-dimethyl-1- benzothiophene-3-carboxamide | 465 |
| Example 6.35 | 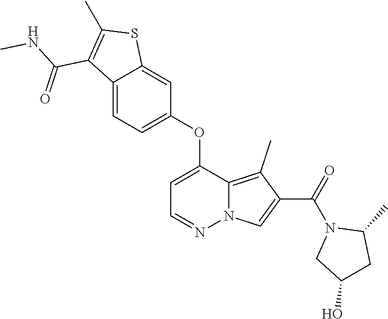 | 6-({6-[(2R,4S)-4-hydroxy-2- methylpyrrolidine-1-carbonyl]- 5-methylpyrrolo[1,2- b]pyridazin-4-yl}oxy)-N,2- dimethyl-1-benzothiophene-3- carboxamide | 479 |
| Example 6.36 | 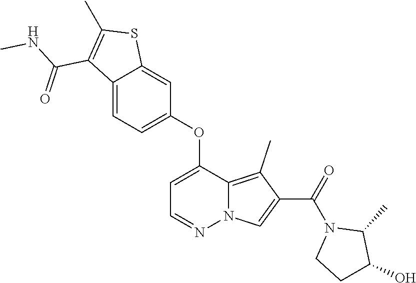 [SMILES: CNC(=O)c1c(C)sc2cc(Oc3ccnn4cc(C(=O)N5CC[C@@H](O)[C@H]5C)c(C)c34)ccc12] | 6-({6-[(2R,3R)-3-hydroxy-2- methylpyrrolidine-1-carbonyl]- 5-methylpyrrolo[1,2- b]pyridazin-4-yl}oxy)-N,2- dimethyl-1-benzothiophene-3- carboxamide | 479 |
| Example 6.37 | 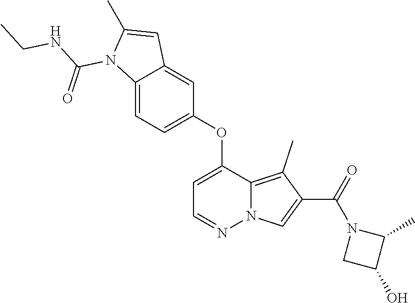 [SMILES: CCNC(=O)n1c(C)cc2cc(Oc3ccnn4cc(C(=O)N5C[C@@H](O)[C@H]5C)c(C)c34)ccc21] | N-ethyl-5-({6-[(2R,3R)-3- hydroxy-2-methylazetidine-1- carbonyl]-5- methylpyrrolo[1,2-b]pyridazin- 4-yl}oxy)-2-methyl-1H-indole- 1-carboxamide | 462 |
| Example 6.38 | 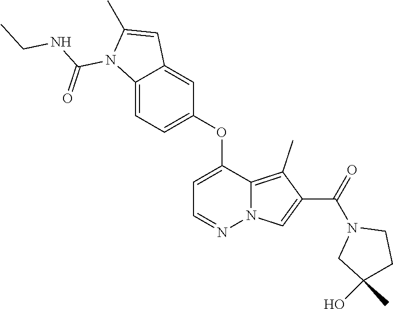 [SMILES: CCNC(=O)n1c(C)cc2cc(Oc3ccnn4cc(C(=O)N5CC[C@](C)(O)C5)c(C)c34)ccc21] | N-ethyl-5-({6-[(3S)-3- hydroxy-3-methylpyrrolidine- 1-carbonyl]-5- methylpyrrolo[1,2-b]pyridazin- 4-yl}oxy)-2-methyl-1H-indole- 1-carboxamide | 476 |
| Example 6.39 | 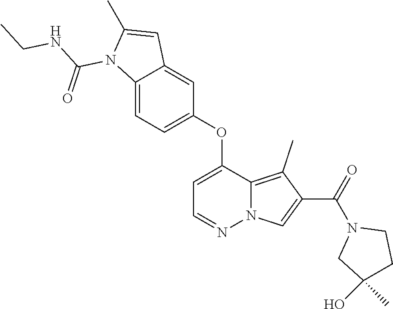 [SMILES: CCNC(=O)n1c(C)cc2cc(Oc3ccnn4cc(C(=O)N5CC[C@@](C)(O)C5)c(C)c34)ccc21] | N-ethyl-5-({6-[(3R)-3- hydroxy-3-methylpyrrolidine- 1-carbonyl]-5- methylpyrrolo[1,2-b]pyridazin- 4-yl}oxy)-2-methyl-1H-indole- 1-carboxamide | 476 |
| Example 6.1 | 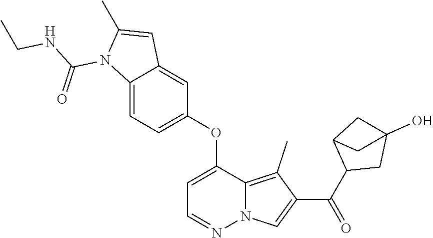 [SMILES: CCNC(=O)n1c(C)cc2cc(Oc3ccnn4cc(C(=O)C5CC6(O)CC5C6)c(C)c34)ccc21] | N-ethyl-5-((6-(4-hydroxy-2- azabicyclo[2.1.1]hexane-2- carbonyl)-5- methylpyrrolo[1,2-b]pyridazin- 4-yl)oxy)-2-methyl-1H-indole- 1-carboxamide | 474 |
| Example 6.2 | 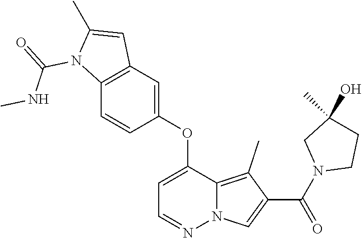 [SMILES: CNC(=O)n1c(C)cc2cc(Oc3ccnn4cc(C(=O)N5CC[C@](C)(O)C5)c(C)c34)ccc21] | (S)-5-((6-(3-hydroxy-3- methylpyrrolidine-1-carbonyl)- 5-methylpyrrolo[1,2- b]pyridazin-4-yl)oxy)-N,2- dimethyl-1H-indole-1- carboxamide | 462 |
| Example 6.3 | 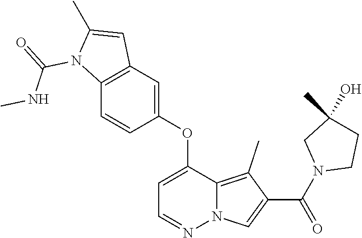 [SMILES: CNC(=O)n1c(C)cc2cc(Oc3ccnn4cc(C(=O)N5CC[C@@](C)(O)C5)c(C)c34)ccc21] | (R)-5-((6-(3-hydroxy-3- methylpyrrolidine-1-carbonyl)- 5-methylpyrrolo[1,2- b]pyridazin-4-yl)oxy)-N,2- dimethyl-1H-indole-1- carboxamide | 462 |
| Example 6.4 | 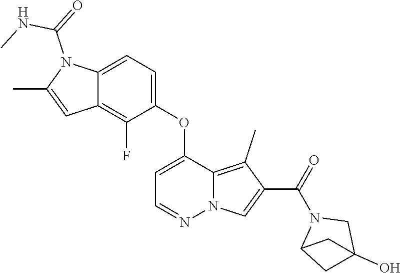 [SMILES: CNC(=O)n1c(C)cc2c(F)c(Oc3ccnn4cc(C(=O)N5CC6(O)CC5C6)c(C)c34)ccc21] | 4-fluoro-5-((6-(4-hydroxy-2- azabicyclo[2.1.1]hexane-2- carbonyl)-5- methylpyrrolo[1,2-b]pyridazin- 4-yl)oxy)-N,2-dimethyl-1H- indole-1-carboxamide | 478 |
| Example 6.5 | 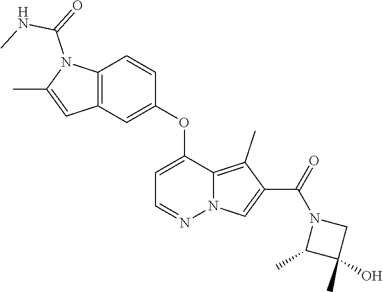 [SMILES: CNC(=O)n1c(C)cc2cc(Oc3ccnn4cc(C(=O)N5C[C@](C)(O)[C@@H]5C)c(C)c34)ccc21] | 5-((6-((2S,3S)-3-hydroxy-2,3- dimethylazetidine-1-carbonyl)- 5-methylpyrrolo[1,2- b]pyridazin-4-yl)oxy)-N,2- dimethyl-1H-indole-1- carboxamide | 462 |

### Preparation of Example 7.1: 65-methyl-N-(4-methyl-1,1-dioxidotetrahydro-2H-thiopyran-4-yl)-4-((2-methyl-3-(methylcarbamoyl)benzofuran-6-yl)oxy)pyrrolo[1,2-b]pyridazine-6-carboxamide

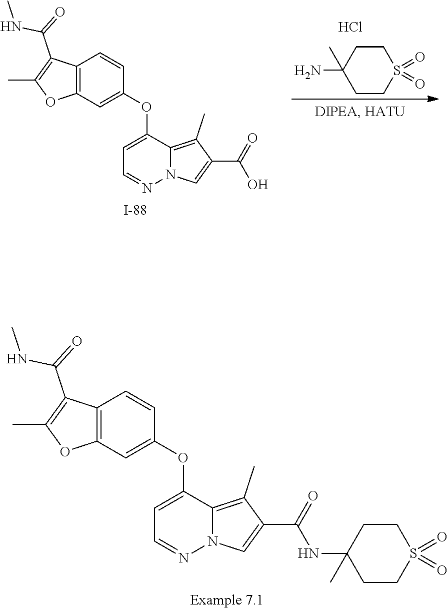

A mixture of 5-methyl-4-((2-methyl-3-(methylcarbamoyl)benzofuran-6-yl)oxy)pyrrolo[1,2-b]pyridazine-6-carboxylic acid (45.0 mg, 0.120 mmol) and HATU (135 mg, 0.360 mmol), DIPEA (0.100 ml, 0.590 mmol), 4-amino-4-methyltetrahydro-2H-thiopyran 1,1-dioxide hydrochloride (23.7 mg, 0.120 mmol) in DMF (1.00 mL) was stirred at 20° C. for 16 hours. The mixture was filtered, and the filtrate was purified by mass-directed reverse phase HPLC (MeCN/water with 0.1% NH₄OH modifier) to afford the desired product. MS: 525 [M+1]⁺. ¹H NMR (400 MHz, CD₃OD) δ 8.14 (s, 1H), 7.84 (s, 1H), 7.82 (d, J=3.2 Hz, 1H), 7.44 (d, J=2.0 Hz, 1H), 7.20 (dd, J=2.0, 8.4 Hz, 1H), 5.64 (d, J=5.2 Hz, 1H), 3.29-3.23 (m, 2H), 3.06-2.99 (m, 2H), 2.97 (s, 3H), 2.93-2.82 (m, 2H), 2.73 (s, 3H), 2.67-2.65 (m, 3H), 2.23-2.14 (m, 2H), 1.56-1.54 (m, 3H).

### Preparation of Example 7.1: 75-((6-((2R,3R)-3-hydroxy-2,3-dimethylazetidine-1-carbonyl)-5-methylpyrrolo[1,2-b]pyridazin-4-yl)oxy)-N,2-dimethyl-1H-indole-1-carboxamide

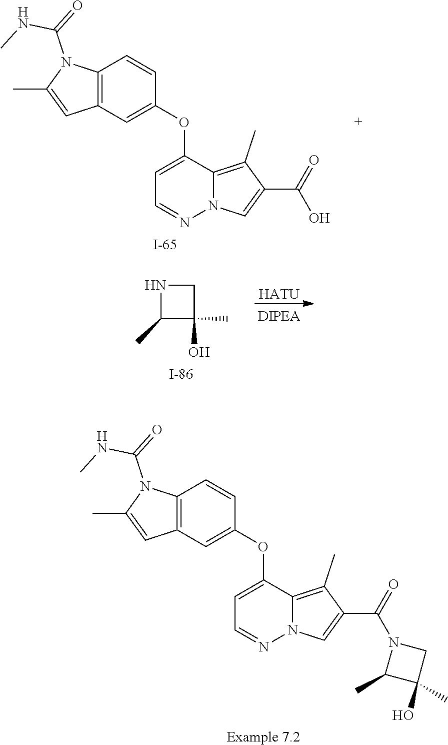

A 40 mL vial was charged with 5-methyl-4-((2-methyl-1-(methylcarbamoyl)-1H-indol-5-yl)oxy)pyrrolo[1,2-b]pyridazine-6-carboxylic acid (I-3) (600 mg, 1.59 mmol), (2R,3R)-2,3-dimethylazetidin-3-ol hydrochloride I-7 (262 mg, 1.90 mmol), HATU (724 mg, 1.90 mmol) and DMF (15.9 mL). DIEA (615 mg, 829 μL, 4.76 mmol) was added, and the resulting reaction mixture stirred at 25° C. for 16 hours. The reaction mixture was diluted with ethyl acetate (20 mL) and washed with aqueous hydrochloric acid (1N, 2×10 mL). The aqueous layer was washed with ethyl acetate (2×10 mL), and the organic layers were combined and washed with aqueous lithium chloride (5%, 2×20 mL), dried (MgSO₄), and filtered. The solvent evaporated under reduced pressure. The crude residue was purified by column chromatography on amino-functionalized silica gel (Biotage Sfär KP-Amino D-Duo 50 M particle size; 11 g prepacked), eluting with 3:1 ethyl acetate/ethanol in hexanes (10% to 50%) to afford 5-((6-((2R,3R)-3-hydroxy-2,3-dimethylazetidine-1-carbonyl)-5-methylpyrrolo[1,2-b]pyridazin-4-yl)oxy)-N,2-dimethyl-1H-indole-1-carboxamide. The isolated product was suspended into 10 mL of isopropyl acetate. The solvent level of the flask was then marked. The suspension was then diluted to 30 mL IPAc total, and this mixture was then sonicated for several minutes. The mixture was then placed on the rotary evaporator with the water bath temperature set to 55° C. Vacuum was pulled very gently while rotating the flask at high rpm, and the solvent level was slowly reduced back down to 10 mL over the course of ˜45 min. A magnetic stirrer was then added to the flask, and the flask was sealed with a rubber septum and placed under a positive pressure of nitrogen. The mixture was then warmed to 55° C. and stirred vigorously. After 30 min the nitrogen inlet was removed and the mixture was stirred at this temperature overnight. In the morning, the mixture was cooled to RT, and solids were collected by vacuum filtration. The crystalline solids were collected into a vial and subjected to extended drying in a vacuum oven (55° C., full house vacuum applied, slight nitrogen bleed to equilibrate pressure to −25 inHg). MS: 462 [M+1]⁺. ¹H NMR (400 MHz, DMSO-d₆) δ 8.27 (m, 1H), 7.94-8.07 (m, 1H), 7.90 (d, J=5.6 Hz, 1H), 7.68 (d, J=8.8 Hz, 1H), 7.40 (d, J=2.0 Hz, 1H), 7.07 (m, 1H), 6.40 (s, 1H), 5.53 (d, J=5.6 Hz, 1H), 4.16 (br. s, 2H), 3.73-3.89 (m, 1H), 2.89 (d, J=4.4 Hz, 3H), 2.64 (br. s, 3H), 2.51 (br. s, 3H), 1.23-1.50 (m, 5H), 1.06 (br. s, 1H).

TABLE 11
Utilizing a method similar to that described for the preparation of Examples 7.1 and
7.2 and the appropriate starting materials, the following examples were prepared:

| | | | Exact |
|---|---|---|---|
| Compound | | | Mass |
| Number | Structure | Name | [M + 1]⁺ |
| Example 7.3 | 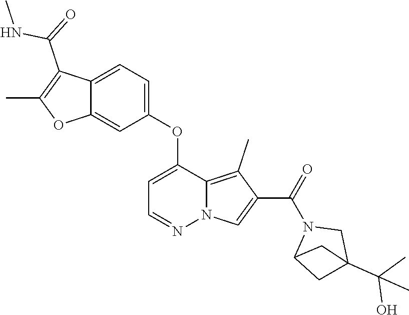 [SMILES: CNC(=O)c1c(C)oc2cc(Oc3ccnn4cc(C(=O)N5CC6(C(C)(C)O)CC5C6)c(C)c34)ccc12] | 6-({6-[4-(2-hydroxypropan-2- yl)-2-azabicyclo[2.1.1]hexane- 2-carbonyl]-5- methylpyrrolo[1,2-b]pyridazin- 4-yl}oxy)-N,2-dimethyl-1- benzofuran-3-carboxamide | 503 |
| Example 7.4 | 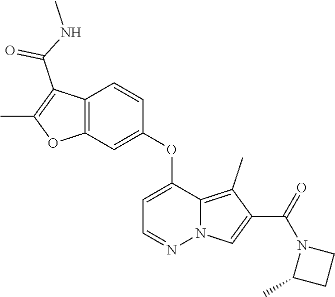 [SMILES: CNC(=O)c1c(C)oc2cc(Oc3ccnn4cc(C(=O)N5CC[C@@H]5C)c(C)c34)ccc12] | N,2-dimethyl-6-({5-methyl-6- [(2S)-2-methylazetidine-1- carbonyl]pyrrolo[1,2- b]pyridazin-4-yl}oxy)-1- benzofuran-3-carboxamide | 433 |
| Example 7.5 | 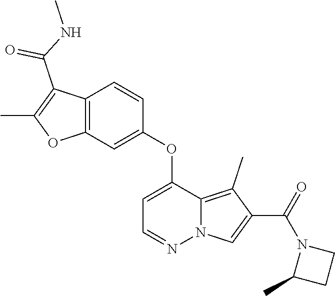 [SMILES: CNC(=O)c1c(C)oc2cc(Oc3ccnn4cc(C(=O)N5CC[C@H]5C)c(C)c34)ccc12] | N,2-dimethyl-6-({5-methyl-6- [(2R)-2-methylazetidine-1- carbonyl]pyrrolo[1,2- b]pyridazin-4-yl}oxy)-1- benzofuran-3-carboxamide | 433 |
| Example 7.6 | 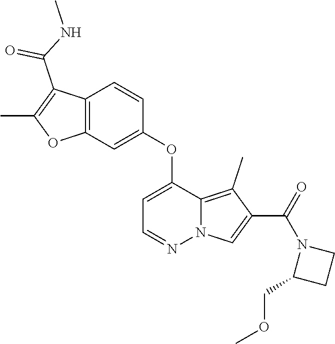 [SMILES: CNC(=O)c1c(C)oc2cc(Oc3ccnn4cc(C(=O)N5CC[C@@H]5COC)c(C)c34)ccc12] | 6-({6-[(2R)-2- (methoxymethyl)azetidine-1- carbonyl]-5-methylpyrrolo[1,2- b]pyridazin-4-yl}oxy)-N,2- dimethyl-1-benzofuran-3- carboxamide | 463 |
| Example 7.7 | 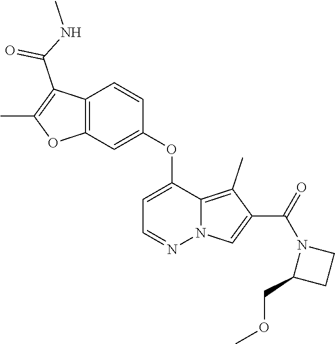 [SMILES: CNC(=O)c1c(C)oc2cc(Oc3ccnn4cc(C(=O)N5CC[C@H]5COC)c(C)c34)ccc12] | 6-({6-[(2S)-2- (methoxymethyl)azetidine-1- carbonyl]-5-methylpyrrolo[1,2- b]pyridazin-4-yl}oxy)-N,2- dimethyl-1-benzofuran-3- carboxamide | 463 |
| Example 7.8 | 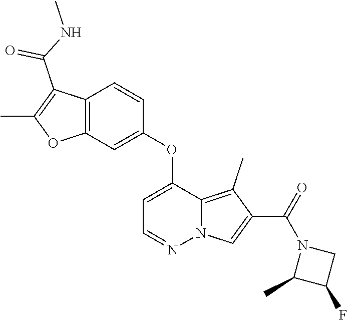 [SMILES: CNC(=O)c1c(C)oc2cc(Oc3ccnn4cc(C(=O)N5C[C@@H](F)[C@H]5C)c(C)c34)ccc12] | 6-({6-[(2R,3R)-3-fluoro-2- methylazetidine-1-carbonyl]-5- methylpyrrolo[1,2-b]pyridazin- 4-yl}oxy)-N,2-dimethyl-1- benzofuran-3-carboxamide | 451 |
| Example 7.9 | 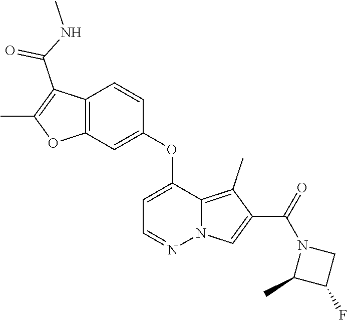 [SMILES: CNC(=O)c1c(C)oc2cc(Oc3ccnn4cc(C(=O)N5C[C@H](F)[C@H]5C)c(C)c34)ccc12] | 6-({6-[(2R,3S)-3-fluoro-2- methylazetidine-1-carbonyl]-5- methylpyrrolo[1,2-b]pyridazin- 4-yl}oxy)-N,2-dimethyl-1- benzofuran-3-carboxamide | 451 |
| Example 7.10 | 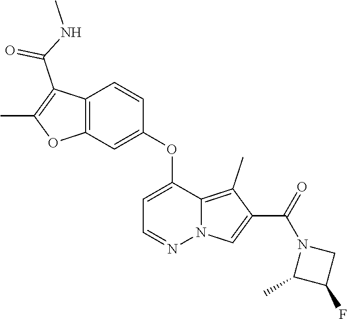 [SMILES: CNC(=O)c1c(C)oc2cc(Oc3ccnn4cc(C(=O)N5C[C@@H](F)[C@@H]5C)c(C)c34)ccc12] | 6-({6-[(2S,3R)-3-fluoro-2- methylazetidine-1-carbonyl]-5- methylpyrrolo[1,2-b]pyridazin- 4-yl}oxy)-N,2-dimethyl-1- benzofuran-3-carboxamide | 451 |
| Example 7.11 | 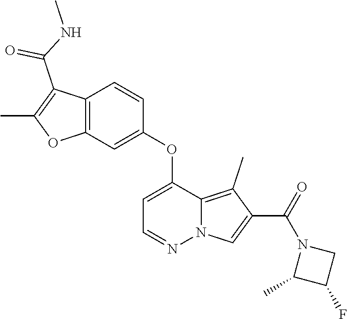 [SMILES: CNC(=O)c1c(C)oc2cc(Oc3ccnn4cc(C(=O)N5C[C@H](F)[C@@H]5C)c(C)c34)ccc12] | 6-({6-[(2S,3S)-3-fluoro-2- methylazetidine-1-carbonyl]-5- methylpyrrolo[1,2-b]pyridazin- 4-yl}oxy)-N,2-dimethyl-1- benzofuran-3-carboxamide | 451 |
| Example 7.12 | 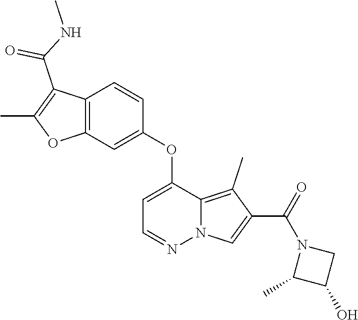 [SMILES: CNC(=O)c1c(C)oc2cc(Oc3ccnn4cc(C(=O)N5C[C@H](O)[C@@H]5C)c(C)c34)ccc12] | 6-({6-[(2S,3S)-3-hydroxy-2- methylazetidine-1-carbonyl]-5- methylpyrrolo[1,2-b]pyridazin- 4-yl}oxy)-N,2-dimethyl-1- benzofuran-3-carboxamide | 449 |
| Example 7.13 | 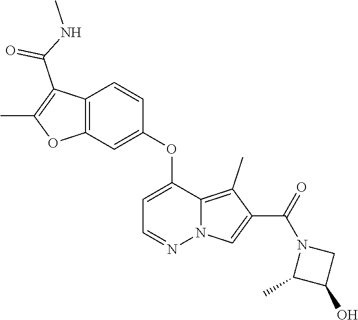 [SMILES: CNC(=O)c1c(C)oc2cc(Oc3ccnn4cc(C(=O)N5C[C@@H](O)[C@@H]5C)c(C)c34)ccc12] | 6-({6-[(2S,3R)-3-hydroxy-2- methylazetidine-1-carbonyl]-5- methylpyrrolo[1,2-b]pyridazin- 4-yl}oxy)-N,2-dimethyl-1- benzofuran-3-carboxamide | 449 |
| Example 7.14 | 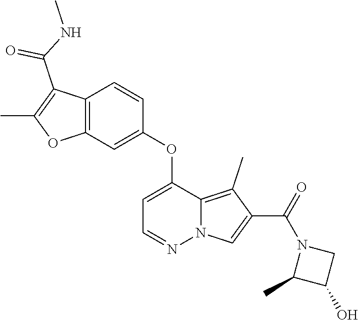 [SMILES: CNC(=O)c1c(C)oc2cc(Oc3ccnn4cc(C(=O)N5C[C@H](O)[C@H]5C)c(C)c34)ccc12] | 6-({6-[(2R,3S)-3-hydroxy-2- methylazetidine-1-carbonyl]-5- methylpyrrolo[1,2-b]pyridazin- 4-yl}oxy)-N,2-dimethyl-1- benzofuran-3-carboxamide | 449 |
| Example 7.15 | 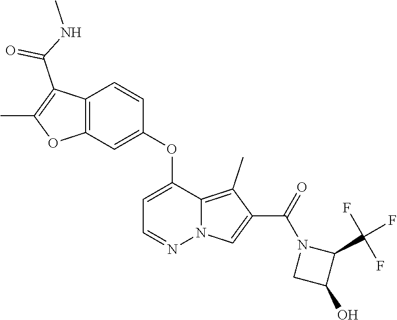 [SMILES: CNC(=O)c1c(C)oc2cc(Oc3ccnn4cc(C(=O)N5C[C@H](O)[C@@H]5C(F)(F)F)c(C)c34)ccc12] | 6-({6-[(2R,3S)-3-hydroxy-2- (trifluoromethyl)azetidine-1- carbonyl]-5-methylpyrrolo[1,2- b]pyridazin-4-yl}oxy)-N,2- dimethyl-1-benzofuran-3- carboxamide | 503 |
| Example 7.16 | 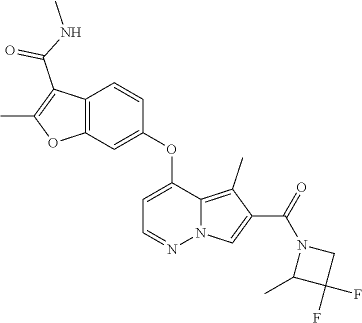 [SMILES: CNC(=O)c1c(C)oc2cc(Oc3ccnn4cc(C(=O)N5CC(F)(F)C5C)c(C)c34)ccc12] | (rac)-6-{[6-(3,3-difluoro-2- methylazetidine-1-carbonyl)-5- methylpyrrolo[1,2-b]pyridazin- 4-yl]oxy}-N,2-dimethyl-1- benzofuran-3-carboxamide | 469 |
| Example 7.17 | 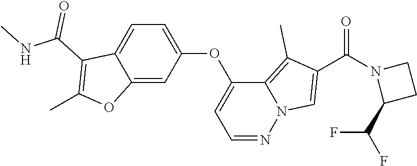 [SMILES: CNC(=O)c1c(C)oc2cc(Oc3ccnn4cc(C(=O)N5CC[C@H]5C(F)F)c(C)c34)ccc12] | 6-({6-[(2S)-2- (difluoromethyl)azetidine-1- carbonyl]-5-methylpyrrolo[1,2- b]pyridazin-4-yl}oxy)-N,2- dimethyl-1-benzofuran-3- carboxamide | 469 |
| Example 7.18 | 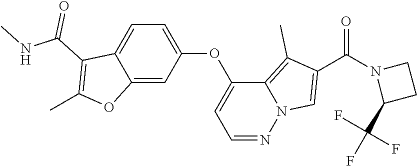 [SMILES: CNC(=O)c1c(C)oc2cc(Oc3ccnn4cc(C(=O)N5CC[C@H]5C(F)(F)F)c(C)c34)ccc12] | N,2-dimethyl-6-({5-methyl-6- [(2S)-2- (trifluoromethyl)azetidine-1- carbonyl]pyrrolo[1,2- b]pyridazin-4-yl}oxy)-1- benzofuran-3-carboxamide | 487 |
| Example 7.19 | 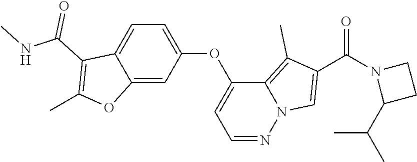 [SMILES: CNC(=O)c1c(C)oc2cc(Oc3ccnn4cc(C(=O)N5CCC5C(C)C)c(C)c34)ccc12] | N,2-dimethyl-6-({5-methyl-6- [2-(propan-2-yl)azetidine-1- carbonyl]pyrrolo[1,2- b]pyridazin-4-yl}oxy)-1- benzofuran-3-carboxamide | 46 |
| Example 7.20 | 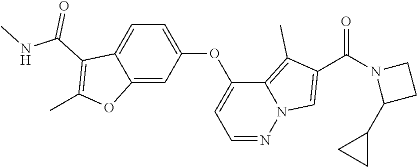 [SMILES: CNC(=O)c1c(C)oc2cc(Oc3ccnn4cc(C(=O)N5CCC5C5CC5)c(C)c34)ccc12] | 6-{[6-(2-cyclopropylazetidine- 1-carbonyl)-5- methylpyrrolo[1,2-b]pyridazin- 4-yl]oxy}-N,2-dimethyl-1- benzofuran-3-carboxamide | 459 |
| Example 7.21 | 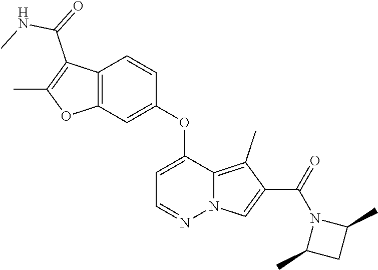 | 6-({6-[(2R,4S)-2,4- dimethylazetidine-1-carbonyl]- 5-methylpyrrolo[1,2- b]pyridazin-4-yl}oxy)-N,2- dimethyl-1-benzofuran-3- carboxamide | 447 |
| Example 7.22 | 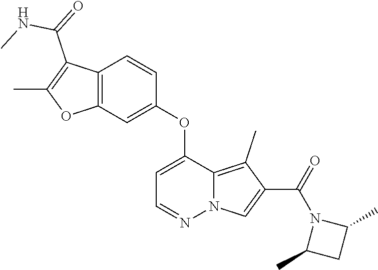 | 6-({6-[(2R,4R)-2,4- dimethylazetidine-1-carbonyl]- 5-methylpyrrolo[1,2- b]pyridazin-4-yl}oxy)-N,2- dimethyl-1-benzofuran-3- carboxamide | 447 |
| Example 7.23 | 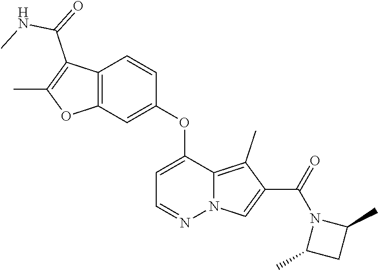 | 6-({6-[(2S,4S)-2,4- dimethylazetidine-1-carbonyl]- 5-methylpyrrolo[1,2- b]pyridazin-4-yl}oxy)-N,2- dimethyl-1-benzofuran-3- carboxamide | 447 |
| Example 7.24 | 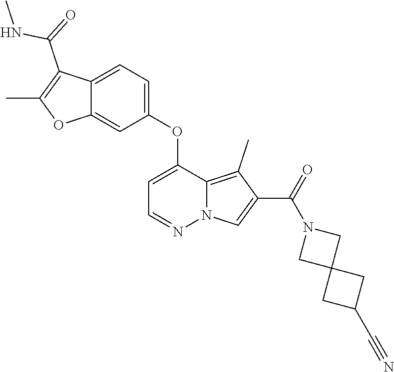 [SMILES: CNC(=O)c1c(C)oc2cc(Oc3ccnn4cc(C(=O)N5CC6(CC(C#N)C6)C5)c(C)c34)ccc12] | 6-{[6-(6-cyano-2- azaspiro[3.3]heptane-2- carbonyl)-5-methylpyrrolo[1,2- b]pyridazin-4-yl]oxy}-N,2- dimethyl-1-benzofuran-3- carboxamide | 484 |
| Example 7.25 | 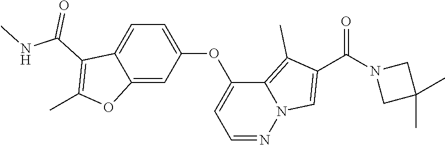 | 6-{[6-(3,3-dimethylazetidine-1- carbonyl)-5-methylpyrrolo[1,2- b]pyridazin-4-yl]oxy}-N,2- dimethyl-1-benzofuran-3- carboxamide | 447 |
| Example 7.26 | 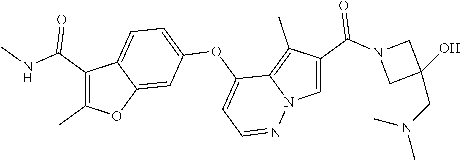 [SMILES: CNC(=O)c1c(C)oc2cc(Oc3ccnn4cc(C(=O)N5CC(O)(CN(C)C)C5)c(C)c34)ccc12] | (rac)-6-[(6-{3- [(dimethylamino)methyl]-3- hydroxyazetidine-1-carbonyl}- 5-methylpyrrolo[1,2- b]pyridazin-4-yl)oxy]-N,2- dimethyl-1-benzofuran-3- carboxamide | 492 |
| Example 7.27 | 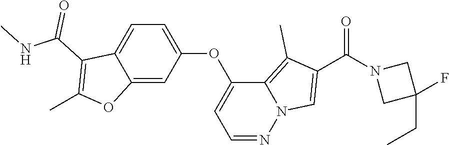 [SMILES: CCC1(F)CN(C(=O)c2cn3nccc(Oc4ccc5c(C(=O)NC)c(C)oc5c4)c3c2C)C1] | (rac)-6-{[6-(3-ethyl-3- fluoroazetidine-1-carbonyl)-5- methylpyrrolo[1,2-b]pyridazin- 4-yl]oxy }-N,2-dimethyl-1- benzofuran-3-carboxamide | 465 |
| Example 7.28 | 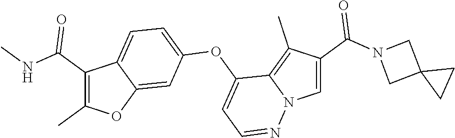 [SMILES: CNC(=O)c1c(C)oc2cc(Oc3ccnn4cc(C(=O)N5CC6(CC6)C5)c(C)c34)ccc12] | 6-{[6-(5-azaspiro[2.3]hexane-5- carbonyl)-5-methylpyrrolo[1,2- b]pyridazin-4-yl]oxy}-N,2- dimethyl-1-benzofuran-3- carboxamide | 445 |
| Example 7.29 | 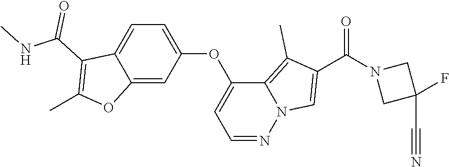 [SMILES: CNC(=O)c1c(C)oc2cc(Oc3ccnn4cc(C(=O)N5CC(F)(C#N)C5)c(C)c34)ccc12] | (rac)-6-{[6-(3-cyano-3- fluoroazetidine-1-carbonyl)-5- methylpyrrolo[1,2-b]pyridazin- 4-yl]oxy}-N,2-dimethyl-1- benzofuran-3-carboxamide | 462 |
| Example 7.30 | 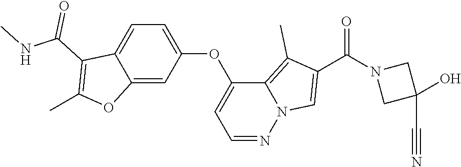 [SMILES: CNC(=O)c1c(C)oc2cc(Oc3ccnn4cc(C(=O)N5CC(O)(C#N)C5)c(C)c34)ccc12] | (rac)-6-{[6-(3-cyano-3- hydroxyazetidine-1-carbonyl)- 5-methylpyrrolo[1,2- b]pyridazin-4-yl]oxy}-N,2- dimethyl-1-benzofuran-3- carboxamide | 460 |
| Example 7.31 | 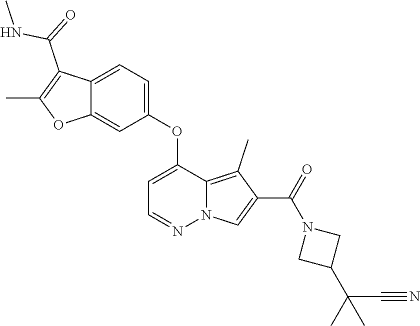 [SMILES: CNC(=O)c1c(C)oc2cc(Oc3ccnn4cc(C(=O)N5CC(C(C)(C)C#N)C5)c(C)c34)ccc12] | 6-({6-[3-(2-cyanopropan-2- yl)azetidine-1-carbonyl]-5- methylpyrrolo[1,2-b]pyridazin- 4-yl}oxy)-N,2-dimethyl-1- benzofuran-3-carboxamide | 486 |
| Example 7.32 | 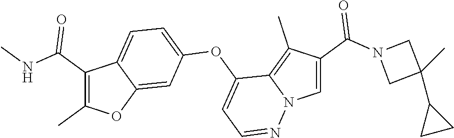 [SMILES: CNC(=O)c1c(C)oc2cc(Oc3ccnn4cc(C(=O)N5CC(C)(C6CC6)C5)c(C)c34)ccc12] | (rac)-6-{[6-(3-cyclopropyl-3- methylazetidine-1-carbonyl)-5- methylpyrrolo[1,2-b]pyridazin- 4-yl]oxy}-N,2-dimethyl-1- benzofuran-3-carboxamide | 473 |
| Example 7.33 | 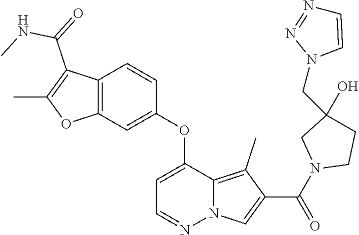 [SMILES: CNC(=O)c1c(C)oc2cc(Oc3ccnn4cc(C(=O)N5CCC(O)(Cn6ccnn6)C5)c(C)c34)ccc12] | (rac)-6-[(6-{3-hydroxy-3-[(1H- 1,2,3-triazol-1- yl)methyl]pyrrolidine-1- carbonyl}-5-methylpyrrolo[1,2- b]pyridazin-4-yl)oxy]-N,2- dimethyl-1-benzofuran-3- carboxamide | 530 |
| Example 7.34 | 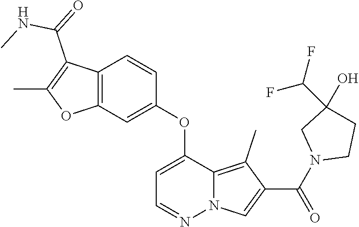 [SMILES: CNC(=O)c1c(C)oc2cc(Oc3ccnn4cc(C(=O)N5CCC(O)(C(F)F)C5)c(C)c34)ccc12] | (rac)-6-({6-[3- (difluoromethyl)-3- hydroxypyrrolidine-1- carbonyl]-5-methylpyrrolo[1,2- b]pyridazin-4-yl}oxy)-N,2- dimethyl-1-benzofuran-3- carboxamide | 499 |
| Example 7.35 | 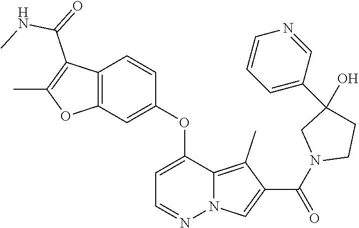 [SMILES: CNC(=O)c1c(C)oc2cc(Oc3ccnn4cc(C(=O)N5CCC(O)(c6cccnc6)C5)c(C)c34)ccc12] | (rac)-6-({6-[3-hydroxy-3- (pyridin-3-yl)pyrrolidine-1- carbonyl]-5-methylpyrrolo[1,2- b]pyridazin-4-yl}oxy)-N,2- dimethyl-1-benzofuran-3- carboxamide | 526 |
| Example 7.36 | 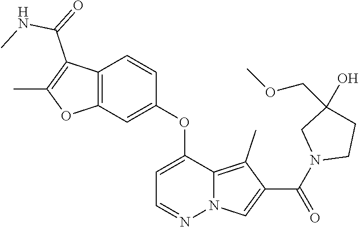 [SMILES: CNC(=O)c1c(C)oc2cc(Oc3ccnn4cc(C(=O)N5CCC(O)(COC)C5)c(C)c34)ccc12] | (rac)-6-({6-[3-hydroxy-3- (methoxymethyl)pyrrolidine-1- carbonyl]-5-methylpyrrolo[1,2- b]pyridazin-4-yl}oxy)-N,2- dimethyl-1-benzofuran-3- carboxamide | 493 |
| Example 7.37 | 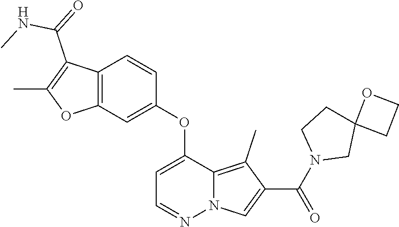 [SMILES: CNC(=O)c1c(C)oc2cc(Oc3ccnn4cc(C(=O)N5CCC6(CCO6)C5)c(C)c34)ccc12] | (rac)-N,2-dimethyl-6-{[5- methyl-6-(1-oxa-6- azaspiro[3.4]octane-6- carbonyl)pyrrolo[1,2- b]pyridazin-4-yl]oxy}-1- benzofuran-3-carboxamide | 475 |
| Example 7.38 | 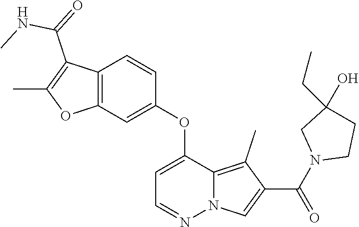 [SMILES: CCC1(O)CCN(C(=O)c2cn3nccc(Oc4ccc5c(C(=O)NC)c(C)oc5c4)c3c2C)C1] | (rac)-6-{[6-(3-ethyl-3- hydroxypyrrolidine-1- carbonyl)-5-methylpyrrolo[1,2- b]pyridazin-4-yl]oxy}-N,2- dimethyl-1-benzofuran-3- carboxamide | 477 |
| Example 7.39 | 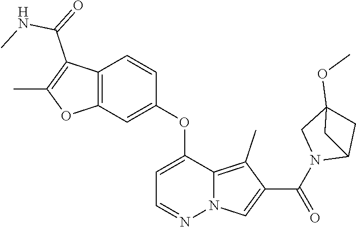 [SMILES: CNC(=O)c1c(C)oc2cc(Oc3ccnn4cc(C(=O)N5CC6(OC)CC5C6)c(C)c34)ccc12] | 6-{[6-(4-methoxy-2- azabicyclo[2.1.1]hexane-2- carbonyl)-5-methylpyrrolo[1,2- b]pyridazin-4-yl]oxy}-N,2- dimethyl-1-benzofuran-3- carboxamide | 475 |
| Example 7.40 | 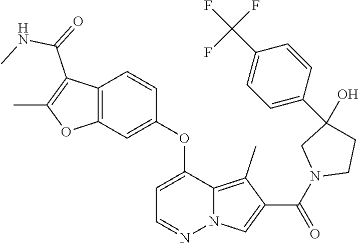 [SMILES: CNC(=O)c1c(C)oc2cc(Oc3ccnn4cc(C(=O)N5CCC(O)(c6ccc(C(F)(F)F)cc6)C5)c(C)c34)ccc12] | (rac)-6-[(6-{3-hydroxy-3-[4- (trifluoromethyl)phenyl] pyrrolidine-1-carbonyl}-5- methylpyrrolo[1,2-b]pyridazin- 4-yl)oxy]-N,2-dimethyl-1- benzofuran-3-carboxamide | 593 |
| Example 7.41 | 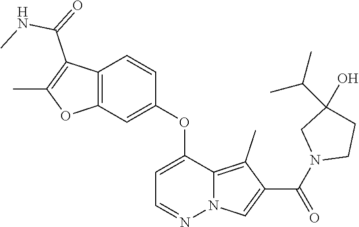 [SMILES: CNC(=O)c1c(C)oc2cc(Oc3ccnn4cc(C(=O)N5CCC(O)(C(C)C)C5)c(C)c34)ccc12] | (rac)-6-({6-[3-hydroxy-3- (propan-2-yl)pyrrolidine-1- carbonyl]-5-methylpyrrolo[1,2- b]pyridazin-4-yl}oxy)-N,2- dimethyl-1-benzofuran-3- carboxamide | 491 |
| Example 7.42 | 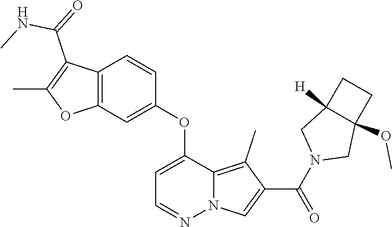 | 6-({6-[(1R,5S)-1-methoxy-3- azabicyclo[3.2.0]heptane-3- carbonyl]-5-methylpyrrolo[1,2- b]pyridazin-4-yl}oxy)-N,2- dimethyl-1-benzofuran-3- carboxamide | 489 |
| Example 7.43 | 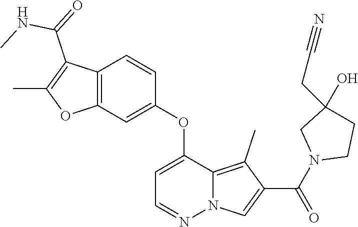 [SMILES: CNC(=O)c1c(C)oc2cc(Oc3ccnn4cc(C(=O)N5CCC(O)(CC#N)C5)c(C)c34)ccc12] | (rac)-6-({6-[3-(cyanomethyl)-3- hydroxypyrrolidine-1- carbonyl]-5-methylpyrrolo[1,2- b]pyridazin-4-yl}oxy)-N,2- dimethyl-1-benzofuran-3- carboxamide | 488 |
| Example 7.44 | 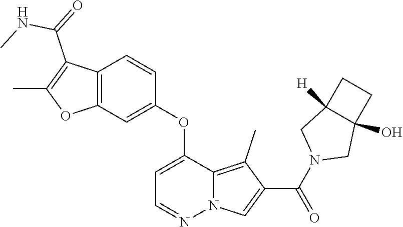 [SMILES: CNC(=O)c1c(C)oc2cc(Oc3ccnn4cc(C(=O)N5C[C@@H]6CC[C@]6(O)C5)c(C)c34)ccc12] | 6-({6-[(1R,5S)-1-hydroxy-3- azabicyclo[3.2.0]heptane-3- carbonyl]-5-methylpyrrolo[1,2- b]pyridazin-4-yl}oxy)-N,2- dimethyl-1-benzofuran-3- carboxamide | 475 |
| Example 7.45 | 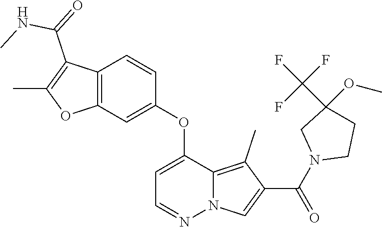 | (rac)-6-({6-[3-methoxy-3- (trifluoromethyl)pyrrolidine-1- carbonyl]-5-methylpyrrolo[1,2- b]pyridazin-4-yl}oxy)-N,2- dimethyl-1-benzofuran-3- carboxamide | 531 |
| Example 7.46 | 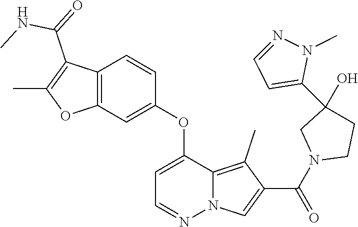 [SMILES: CNC(=O)c1c(C)oc2cc(Oc3ccnn4cc(C(=O)N5CCC(O)(c6ccnn6C)C5)c(C)c34)ccc12] | (rac)-6-({6-[3-hydroxy-3-(1- methyl-1H-pyrazol-5- yl)pyrrolidine-1-carbonyl]-5- methylpyrrolo[1,2-b]pyridazin- 4-yl}oxy)-N,2-dimethyl-1- benzofuran-3-carboxamide | 529 |
| Example 7.47 | 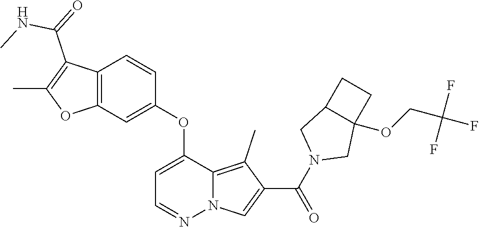 [SMILES: CNC(=O)c1c(C)oc2cc(Oc3ccnn4cc(C(=O)N5CC6CCC6(OCC(F)(F)F)C5)c(C)c34)ccc12] | (rac)-N,2-dimethyl-6-({5- methyl-6-[1-(2,2,2- trifluoroethoxy)-3- azabicyclo[3.2.0]heptane-3- carbonyl]pyrrolo[1,2- b]pyridazin-4-yl}oxy)-1- benzofuran-3-carboxamide | 557 |
| Example 7.48 | 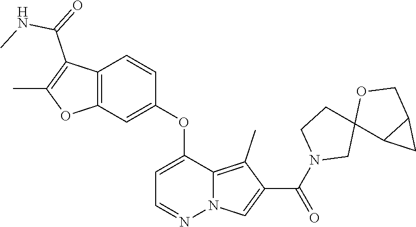 [SMILES: CNC(=O)c1c(C)oc2cc(Oc3ccnn4cc(C(=O)N5CCC6(C5)OCC5CC56)c(C)c34)ccc12] | N,2-dimethyl-6-{[5-methyl-6- (1′H-spiro[3- oxabicyclo[3.1.0]hexane-2,3′- pyrrolidine]-1′- carbonyl)pyrrolo[1,2- b]pyridazin-4-yl]oxy}-1- benzofuran-3-carboxamide | 501 |
| Example 7.49 | 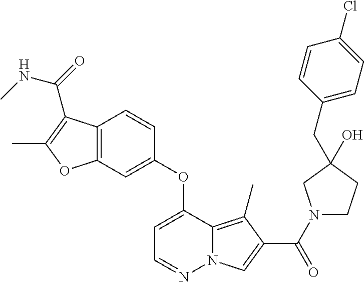 [SMILES: CNC(=O)c1c(C)oc2cc(Oc3ccnn4cc(C(=O)N5CCC(O)(Cc6ccc(Cl)cc6)C5)c(C)c34)ccc12] | (rac)-6-[(6-{3-[(4- chlorophenyl)methyl]-3- hydroxypyrrolidine-1- carbonyl}-5-methylpyrrolo[1,2- b]pyridazin-4-yl)oxy]-N,2- dimethyl-1-benzofuran-3- carboxamide | 573 |
| Example 7.50 | 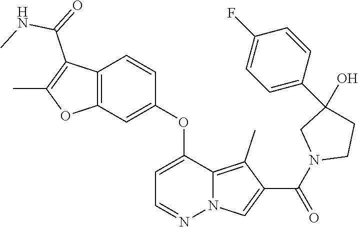 [SMILES: CNC(=O)c1c(C)oc2cc(Oc3ccnn4cc(C(=O)N5CCC(O)(c6ccc(F)cc6)C5)c(C)c34)ccc12] | (rac)-6-({6-[3-(4-fluorophenyl)- 3-hydroxypyrrolidine-1- carbonyl]-5-methylpyrrolo[1,2- b]pyridazin-4-yl}oxy)-N,2- dimethyl-1-benzofuran-3- carboxamide | 543 |
| Example 7.51 | 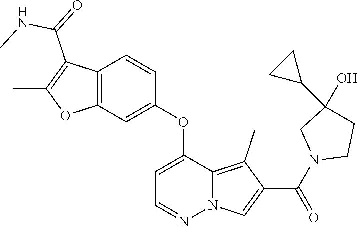 [SMILES: CNC(=O)c1c(C)oc2cc(Oc3ccnn4cc(C(=O)N5CCC(O)(C6CC6)C5)c(C)c34)ccc12] | (rac)-6-{[6-(3-cyclopropyl-3- hydroxypyrrolidine-1- carbonyl)-5-methylpyrrolo[1,2- b]pyridazin-4-yl]oxy}-N,2- dimethyl-1-benzofuran-3- carboxamide | 489 |
| Example 7.52 | 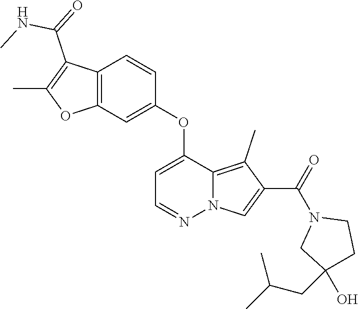 [SMILES: CNC(=O)c1c(C)oc2cc(Oc3ccnn4cc(C(=O)N5CCC(O)(CC(C)C)C5)c(C)c34)ccc12] | (rac)-6-({6-[3-hydroxy-3-(2- methylpropyl)pyrrolidine-1- carbonyl]-5-methylpyrrolo[1,2- b]pyridazin-4-yl}oxy)-N,2- dimethyl-1-benzofuran-3- carboxamide | 505 |
| Example 7.53 | 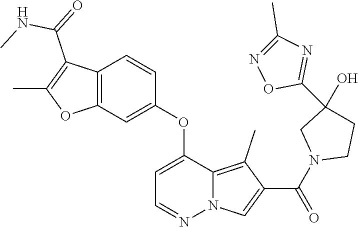 [SMILES: CNC(=O)c1c(C)oc2cc(Oc3ccnn4cc(C(=O)N5CCC(O)(c6nc(C)no6)C5)c(C)c34)ccc12] | (rac)-6-({6-[3-hydroxy-3-(3- methyl-1,2,4-oxadiazol-5- yl)pyrrolidine-1-carbonyl]-5- methylpyrrolo[1,2-b]pyridazin- 4-yl}oxy)-N,2-dimethyl-1- benzofuran-3-carboxamide | 531 |
| Example 7.54 | 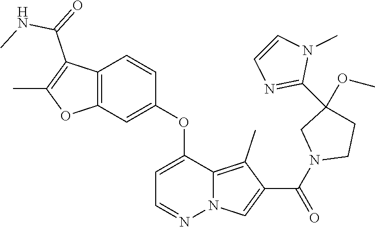 | (rac)-6-({6-[3-methoxy-3-(1- methyl-1H-imidazol-2- yl)pyrrolidine-1-carbonyl]-5- methylpyrrolo[1,2-b]pyridazin- 4-yl}oxy)-N,2-dimethyl-1- benzofuran-3-carboxamide | 543 |
| Example 7.55 | 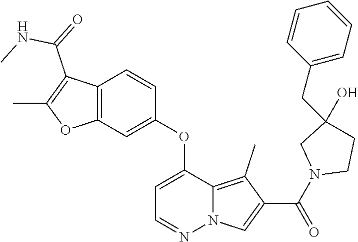 [SMILES: CNC(=O)c1c(C)oc2cc(Oc3ccnn4cc(C(=O)N5CCC(O)(Cc6ccccc6)C5)c(C)c34)ccc12] | (rac)-6-{[6-(3-benzyl-3- hydroxypyrrolidine-1- carbonyl)-5-methylpyrrolo[1,2- b]pyridazin-4-yl]oxy}-N,2- dimethyl-1-benzofuran-3- carboxamide | 539 |
| Example 7.56 | 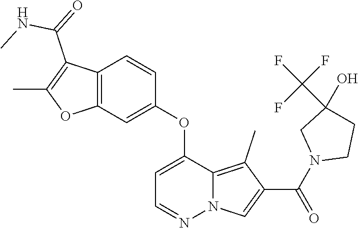 [SMILES: CNC(=O)c1c(C)oc2cc(Oc3ccnn4cc(C(=O)N5CCC(O)(C(F)(F)F)C5)c(C)c34)ccc12] | (rac)-6-({6-[3-hydroxy-3- (trifluoromethyl)pyrrolidine-1- carbonyl]-5-methylpyrrolo[1,2- b]pyridazin-4-yl}oxy)-N,2- dimethyl-1-benzofuran-3- carboxamide | 517 |
| Example 7.57 | 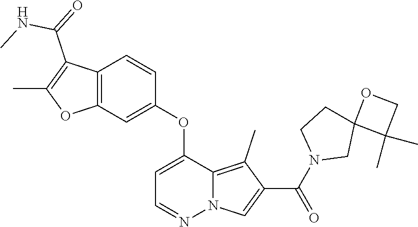 | (rac)-6-{[6-(3,3-dimethyl-1- oxa-6-azaspiro[3.4]octane-6- carbonyl)-5-methylpyrrolo[1,2- b]pyridazin-4-yl]oxy}-N,2- dimethyl-1-benzofuran-3- carboxamide | 503 |
| Example 7.58 | 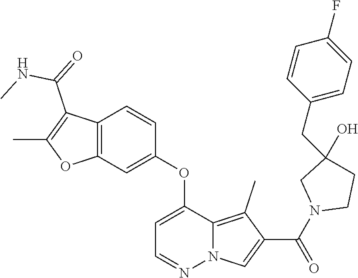 [SMILES: CNC(=O)c1c(C)oc2cc(Oc3ccnn4cc(C(=O)N5CCC(O)(Cc6ccc(F)cc6)C5)c(C)c34)ccc12] | (rac)-6-[(6-{3-[(4- fluorophenyl)methyl]-3- hydroxypyrrolidine-1- carbonyl}-5-methylpyrrolo[1,2- b]pyridazin-4-yl)oxy]-N,2- dimethyl-1-benzofuran-3- carboxamide | 557 |
| Example 7.59 | 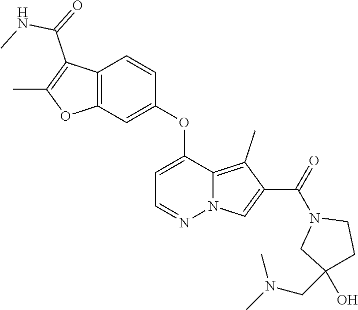 [SMILES: CNC(=O)c1c(C)oc2cc(Oc3ccnn4cc(C(=O)N5CCC(O)(CN(C)C)C5)c(C)c34)ccc12] | (rac)-6-[(6-{3- [(dimethylamino)methyl]-3- hydroxypyrrolidine-1- carbonyl}-5-methylpyrrolo[1,2- b]pyridazin-4-yl)oxy]-N,2- dimethyl-1-benzofuran-3- carboxamide | 506 |
| Example 7.60 | 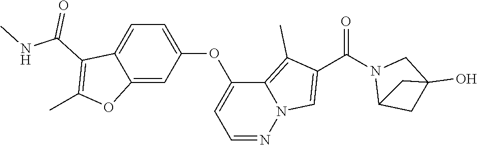 [SMILES: CNC(=O)c1c(C)oc2cc(Oc3ccnn4cc(C(=O)N5CC6(O)CC5C6)c(C)c34)ccc12] | 6-{[6-(4-hydroxy-2- azabicyclo[2.1.1]hexane-2- carbonyl)-5-methylpyrrolo[1,2- b]pyridazin-4-yl]oxy}-N,2- dimethyl-1-benzofuran-3- carboxamide | 461 |
| Example 7.61 | 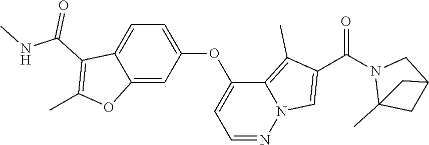 [SMILES: CNC(=O)c1c(C)oc2cc(Oc3ccnn4cc(C(=O)N5CC6CC5(C)C6)c(C)c34)ccc12] | N,2-dimethyl-6-{[5-methyl-6- (1-methyl-2- azabicyclo[2.1.1]hexane-2- carbonyl)pyrrolo[1,2- b]pyridazin-4-ylJoxy}-1- benzofuran-3-carboxamide | 459 |
| Example 7.62 | 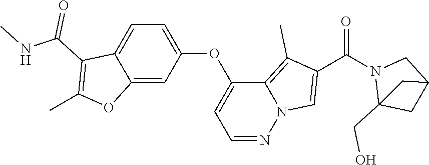 [SMILES: CNC(=O)c1c(C)oc2cc(Oc3ccnn4cc(C(=O)N5CC6CC5(CO)C6)c(C)c34)ccc12] | 6-({6-[1-(hydroxymethyl)-2- azabicyclo[2.1.1]hexane-2- carbonyl]-5-methylpyrrolo[1,2- b]pyridazin-4-yl}oxy)-N,2- dimethyl-1-benzofuran-3- carboxamide | 475 |
| Example 7.63 | 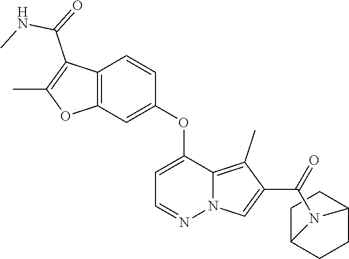 [SMILES: CNC(=O)c1c(C)oc2cc(Oc3ccnn4cc(C(=O)N5C6CCC5CC6)c(C)c34)ccc12] | 6-{[6-(7- azabicyclo[2.2.1]heptane-7- carbonyl)-5-methylpyrrolo[1,2- b]pyridazin-4-yl]oxy}-N,2- dimethyl-1-benzofuran-3- carboxamide | 459 |
| Example 7.64 | 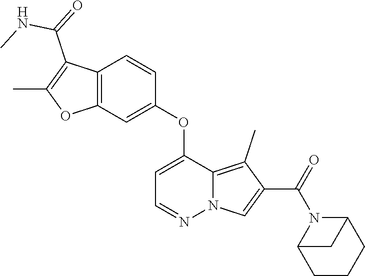 [SMILES: CNC(=O)c1c(C)oc2cc(Oc3ccnn4cc(C(=O)N5C6CCCC5C6)c(C)c34)ccc12] | 6-{[6-(6- azabicyclo[3.1.1]heptane-6- carbonyl)-5-methylpyrrolo[1,2- b]pyridazin-4-yl]oxy}-N,2- dimethyl-1-benzofuran-3- carboxamide | 459 |
| Example 7.65 | 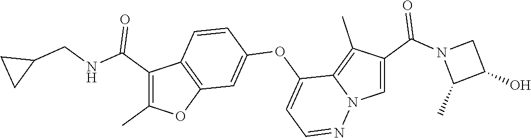 [SMILES: Cc1oc2cc(Oc3ccnn4cc(C(=O)N5C[C@H](O)[C@@H]5C)c(C)c34)ccc2c1C(=O)NCC1CC1] | N-(cyclopropylmethyl)-6- ({6-[(2S,3S)-3-hydroxy-2- methylazetidine-1-carbonyl]-5- methylpyrrolo[1,2-b]pyridazin- 4-yl}oxy)-2-methyl-1- benzofuran-3-carboxamide | 489 |
| Example 7.66 | 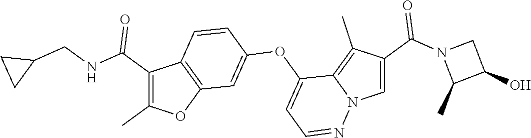 [SMILES: Cc1oc2cc(Oc3ccnn4cc(C(=O)N5C[C@@H](O)[C@H]5C)c(C)c34)ccc2c1C(=O)NCC1CC1] | N-(cyclopropylmethyl)-6-({6- [(2R,3R)-3-hydroxy-2- methylazetidine-1-carbonyl]-5- methylpyrrolo[1,2-b]pyridazin- 4-yl}oxy)-2-methyl-1- benzofuran-3-carboxamide | 489 |
| Example 7.67 | 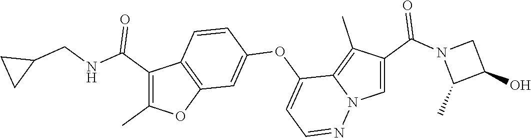 [SMILES: Cc1oc2cc(Oc3ccnn4cc(C(=O)N5C[C@@H](O)[C@@H]5C)c(C)c34)ccc2c1C(=O)NCC1CC1] | N-(cyclopropylmethyl)-6-({6- [(2S,3R)-3-hydroxy-2- methylazetidine-1-carbonyl]-5- methylpyrrolo[1,2-b]pyridazin- 4-yl}oxy)-2-methyl-1- benzofuran-3-carboxamide | 489 |
| Example 7.68 | 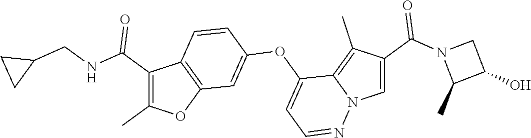 [SMILES: Cc1oc2cc(Oc3ccnn4cc(C(=O)N5C[C@H](O)[C@H]5C)c(C)c34)ccc2c1C(=O)NCC1CC1] | N-(cyclopropylmethyl)-6-({6- [(2R,3S)-3-hydroxy-2- methylazetidine-1-carbonyl]-5- methylpyrrolo[1,2-b]pyridazin- 4-yl}oxy)-2-methyl-1- benzofuran-3-carboxamide | 489 |
| Example 7.69 | 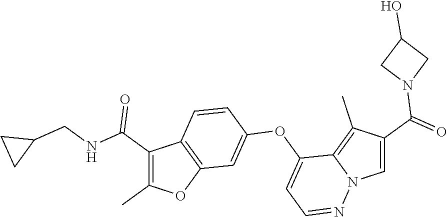 [SMILES: Cc1oc2cc(Oc3ccnn4cc(C(=O)N5CC(O)C5)c(C)c34)ccc2c1C(=O)NCC1CC1] | N-(cyclopropylmethyl)-6-{[6- (3-hydroxyazetidine-1- carbonyl)-5-methylpyrrolo[1,2- b]pyridazin-4-yl]oxy}-2- methyl-1-benzofuran-3- carboxamide | 475 |
| Example 7.70 | 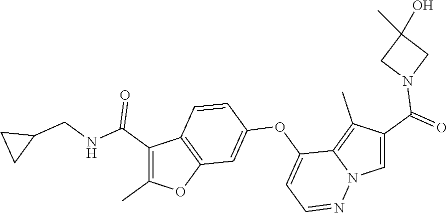 [SMILES: Cc1oc2cc(Oc3ccnn4cc(C(=O)N5CC(C)(O)C5)c(C)c34)ccc2c1C(=O)NCC1CC1] | N-(cyclopropylmethyl)-6-{[6- (3-hydroxy-3-methylazetidine- 1-carbonyl)-5- methylpyrrolo[1,2-b]pyridazin- 4-yl]oxy}-2-methyl-1- benzofuran-3-carboxamide | 489 |
| Example 7.71 | 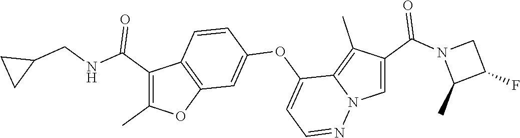 [SMILES: Cc1oc2cc(Oc3ccnn4cc(C(=O)N5C[C@H](F)[C@H]5C)c(C)c34)ccc2c1C(=O)NCC1CC1] | N-(cyclopropylmethyl)-6-({6- [(2R,3S)-3-fluoro-2- methylazetidine-1-carbonyl]-5- methylpyrrolo[1,2-b]pyridazin- 4-yl}oxy)-2-methyl-1- benzofuran-3-carboxamide | 491 |
| Example 7.72 | 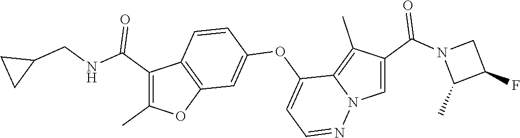 [SMILES: Cc1oc2cc(Oc3ccnn4cc(C(=O)N5C[C@@H](F)[C@@H]5C)c(C)c34)ccc2c1C(=O)NCC1CC1] | N-(cyclopropylmethyl)-6-({6- [(2S,3R)-3-fluoro-2- methylazetidine-1-carbonyl]-5- methylpyrrolo[1,2-b]pyridazin- 4-yl}oxy)-2-methyl-1- benzofuran-3-carboxamide | 491 |
| Example 7.73 | 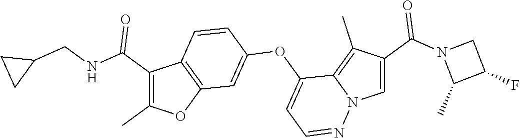 [SMILES: Cc1oc2cc(Oc3ccnn4cc(C(=O)N5C[C@H](F)[C@@H]5C)c(C)c34)ccc2c1C(=O)NCC1CC1] | N-(cyclopropylmethyl)-6-({6- [(2S,3S)-3-fluoro-2- methylazetidine-1-carbonyl]-5- methylpyrrolo[1,2-b]pyridazin- 4-yl}oxy)-2-methyl-1- benzofuran-3-carboxamide | 491 |
| Example 7.74 | 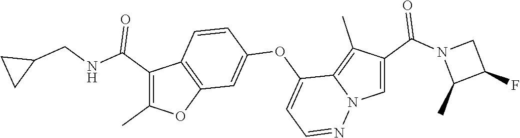 [SMILES: Cc1oc2cc(Oc3ccnn4cc(C(=O)N5C[C@@H](F)[C@H]5C)c(C)c34)ccc2c1C(=O)NCC1CC1] | N-(cyclopropylmethyl)-6-({6- [(2R,3R)-3-fluoro-2- methylazetidine-1-carbonyl]-5- methylpyrrolo[1,2-b]pyridazin- 4-yl}oxy)-2-methyl-1- benzofuran-3-carboxamide | 491 |
| Example 7.75 | 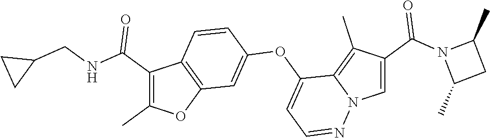 [SMILES: Cc1oc2cc(Oc3ccnn4cc(C(=O)N5[C@@H](C)C[C@@H]5C)c(C)c34)ccc2c1C(=O)NCC1CC1] | N-(cyclopropylmethyl)-6-({6- [(2S,4S)-2,4-dimethylazetidine- 1-carbonyl]-5- methylpyrrolo[1,2-b]pyridazin- 4-yl}oxy)-2-methyl-1- benzofuran-3-carboxamide | 487 |
| Example 7.76 | 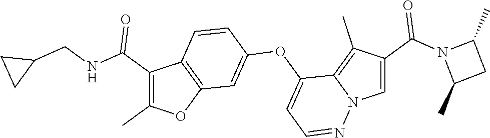 [SMILES: Cc1oc2cc(Oc3ccnn4cc(C(=O)N5[C@H](C)C[C@H]5C)c(C)c34)ccc2c1C(=O)NCC1CC1] | N-(cyclopropylmethyl)-6-({6- [(2R,4R)-2,4-dimethylazetidine- 1-carbonyl]-5- methylpyrrolo[1,2-b]pyridazin- 4-yl}oxy)-2-methyl-1- benzofuran-3-carboxamide | 487 |
| Example 7.77 | 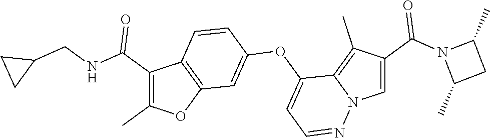 [SMILES: Cc1oc2cc(Oc3ccnn4cc(C(=O)N5[C@H](C)C[C@@H]5C)c(C)c34)ccc2c1C(=O)NCC1CC1] | N-(cyclopropylmethyl)-6-({6- [(2R,4S)-2,4-dimethylazetidine- 1-carbonyl]-5- methylpyrrolo[1,2-b]pyridazin- 4-yl}oxy)-2-methyl-1- benzofuran-3-carboxamide | 487 |
| Example 7.78 | 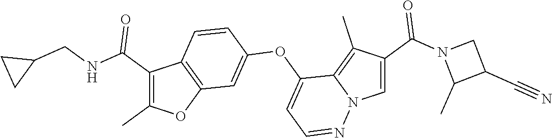 [SMILES: Cc1oc2cc(Oc3ccnn4cc(C(=O)N5CC(C#N)C5C)c(C)c34)ccc2c1C(=O)NCC1CC1] | 6-{[6-(3-cyano-2- methylazetidine-1-carbonyl)-5- methylpyrrolo[1,2-b]pyridazin- 4-yl]oxy}-N- (cyclopropylmethyl)-2-methyl- 1-benzofuran-3-carboxamide | 498 |
| Example 7.79 | 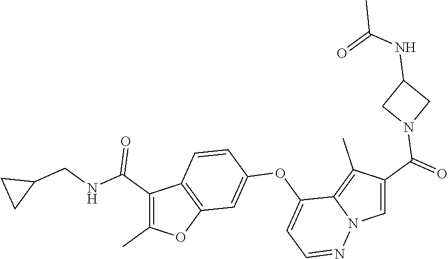 [SMILES: CC(=O)NC1CN(C(=O)c2cn3nccc(Oc4ccc5c(C(=O)NCC6CC6)c(C)oc5c4)c3c2C)C1] | 6-({6-[3- (acetylamino)azetidine-1- carbonyl]-5-methylpyrrolo[1,2- b]pyridazin-4-yl}oxy)-N- (cyclopropylmethyl)-2-methyl- 1-benzofuran-3-carboxamide | 516 |
| Example 7.80 | 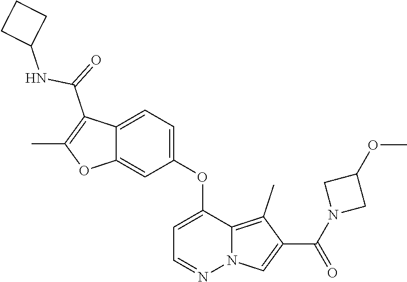 [SMILES: COC1CN(C(=O)c2cn3nccc(Oc4ccc5c(C(=O)NC6CCC6)c(C)oc5c4)c3c2C)C1] | N-cyclobutyl-6-{[6-(3- methoxyazetidine-1-carbonyl)- 5-methylpyrrolo[1,2- b]pyridazin-4-yl]oxy}-2- methyl-1-benzofuran-3- carboxamide | 489 |
| Example 7.81 | 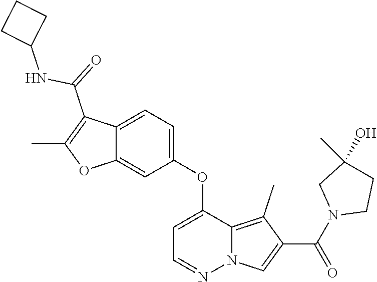 [SMILES: Cc1oc2cc(Oc3ccnn4cc(C(=O)N5CC[C@@](C)(O)C5)c(C)c34)ccc2c1C(=O)NC1CCC1] | N-cyclobutyl-6-({6-[(3R)-3- hydroxy-3-methylpyrrolidine-1- carbonyl]-5-methylpyrrolo[1,2- b]pyridazin-4-yl}oxy)-2- methyl-1-benzofuran-3- carboxamide | 503 |
| Example 7.82 | 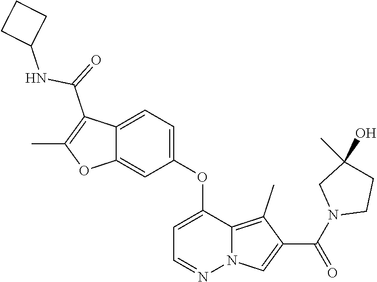 [SMILES: Cc1oc2cc(Oc3ccnn4cc(C(=O)N5CC[C@](C)(O)C5)c(C)c34)ccc2c1C(=O)NC1CCC1] | N-cyclobutyl-6-({6-[(3S)-3- hydroxy-3-methylpyrrolidine-1- carbonyl]-5-methylpyrrolo[1,2- b]pyridazin-4-yl}oxy)-2- methyl-1-benzofuran-3- carboxamide | 503 |
| Example 7.83 | 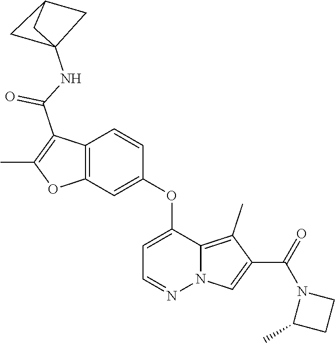 [SMILES: Cc1oc2cc(Oc3ccnn4cc(C(=O)N5CC[C@@H]5C)c(C)c34)ccc2c1C(=O)NC12CC(C1)C2] | N-(bicyclo[1.1.1]pentan-1-yl)- 2-methyl-6-({5-methyl-6-[(2S)- 2-methylazetidine-1- carbonyl]pyrrolo[1,2- b]pyridazin-4-yl}oxy)-1- benzofuran-3-carboxamide | 485 |
| Example 7.84 | 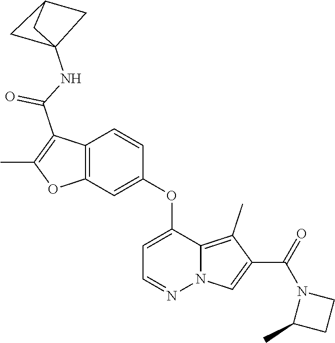 [SMILES: Cc1oc2cc(Oc3ccnn4cc(C(=O)N5CC[C@H]5C)c(C)c34)ccc2c1C(=O)NC12CC(C1)C2] | N-(bicyclo[1.1.1]pentan-1-yl)- 2-methyl-6-({5-methyl-6-[(2R)- 2-methylazetidine-1- carbonyl]pyrrolo[1,2- b]pyridazin-4-yl}oxy)-1- benzofuran-3-carboxamide | 485 |
| Example 7.85 | 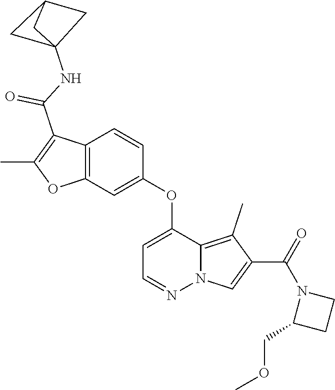 [SMILES: COC[C@H]1CCN1C(=O)c1cn2nccc(Oc3ccc4c(C(=O)NC56CC(C5)C6)c(C)oc4c3)c2c1C] | N-(bicyclo[1.1.1]pentan-1-yl)- 6-({6-[(2R)-2- (methoxymethyl)azetidine-1- carbonyl]-5-methylpyrrolo[1,2- b]pyridazin-4-yl}oxy)-2- methyl-1-benzofuran-3- carboxamide | 515 |
| Example 7.86 | 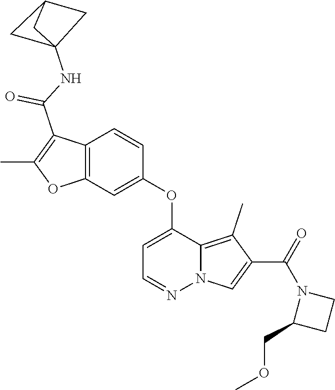 [SMILES: COC[C@@H]1CCN1C(=O)c1cn2nccc(Oc3ccc4c(C(=O)NC56CC(C5)C6)c(C)oc4c3)c2c1C] | N-(bicyclo[1.1.1]pentan-1-yl)- 6-({6-[(2S)-2- (methoxymethyl)azetidine-1- carbonyl]-5-methylpyrrolo[1,2- b]pyridazin-4-yl}oxy)-2- methyl-1-benzofuran-3- carboxamide | 515 |
| Example 7.87 | 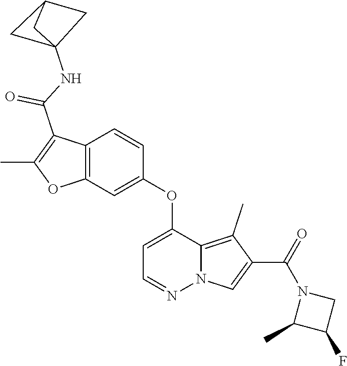 [SMILES: Cc1oc2cc(Oc3ccnn4cc(C(=O)N5C[C@@H](F)[C@H]5C)c(C)c34)ccc2c1C(=O)NC12CC(C1)C2] | N-(bicyclo[1.1.1]pentan-1-yl)- 6-({6-[(2R,3R)-3-fluoro-2- methylazetidine-1-carbonyl]-5- methylpyrrolo[1,2-b]pyridazin- 4-yl}oxy)-2-methyl-1- benzofuran-3-carboxamide | 503 |
| Example 7.88 | 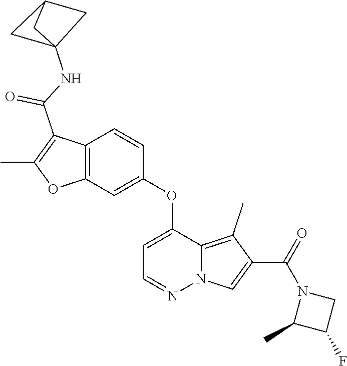 [SMILES: Cc1oc2cc(Oc3ccnn4cc(C(=O)N5C[C@H](F)[C@H]5C)c(C)c34)ccc2c1C(=O)NC12CC(C1)C2] | N-(bicyclo[1.1.1]pentan-1-yl)- 6-({6-[(2R,3S)-3-fluoro-2- methylazetidine-1-carbonyl]-5- methylpyrrolo[1,2-b]pyridazin- 4-yl}oxy)-2-methyl-1- benzofuran-3-carboxamide | 503 |
| Example 7.89 | 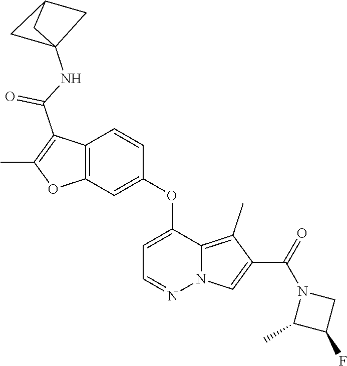 [SMILES: Cc1oc2cc(Oc3ccnn4cc(C(=O)N5C[C@@H](F)[C@@H]5C)c(C)c34)ccc2c1C(=O)NC12CC(C1)C2] | N-(bicyclo[1.1.1]pentan-1-yl)- 6-({6-[(2S,3R)-3-fluoro-2- methylazetidine-1-carbonyl]-5- methylpyrrolo[1,2-b]pyridazin- 4-yl}oxy)-2-methyl-1- benzofuran-3-carboxamide | 503 |
| Example 7.90 | 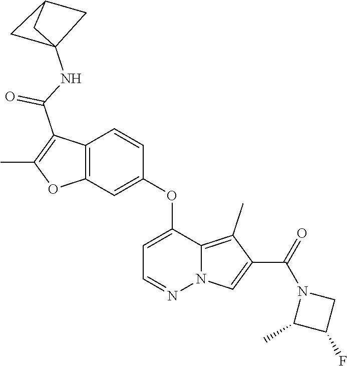 [SMILES: Cc1oc2cc(Oc3ccnn4cc(C(=O)N5C[C@H](F)[C@@H]5C)c(C)c34)ccc2c1C(=O)NC12CC(C1)C2] | N-(bicyclo[1.1.1]pentan-1-yl)- 6-({6-[(2S,3S)-3-fluoro-2- methylazetidine-1-carbonyl]-5- methylpyrrolo[1,2-b]pyridazin- 4-yl}oxy)-2-methyl-1- benzofuran-3-carboxamide | 503 |
| Example 7.91 | 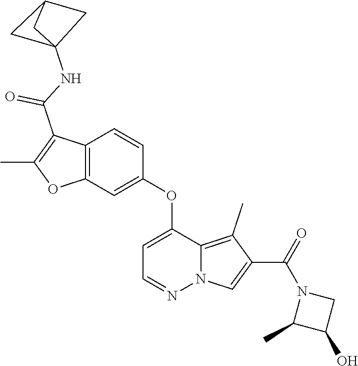 [SMILES: Cc1oc2cc(Oc3ccnn4cc(C(=O)N5C[C@@H](O)[C@H]5C)c(C)c34)ccc2c1C(=O)NC12CC(C1)C2] | N-(bicyclo[1.1.1]pentan-1-yl)- 6-({6-[(2R,3R)-3-hydroxy-2- methylazetidine-1-carbonyl]-5- methylpyrrolo[1,2-b]pyridazin- 4-yl}oxy)-2-methyl-1- benzofuran-3-carboxamide | 501 |
| Example 7.92 | 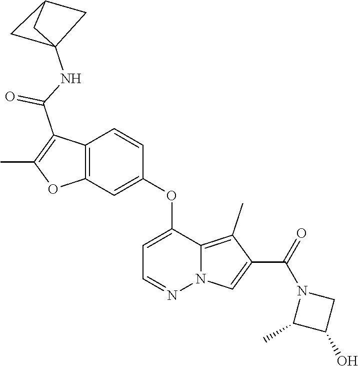 [SMILES: Cc1oc2cc(Oc3ccnn4cc(C(=O)N5C[C@H](O)[C@@H]5C)c(C)c34)ccc2c1C(=O)NC12CC(C1)C2] | N-(bicyclo[1.1.1]pentan-1-yl)- 6-({6-[(2S,3S)-3-hydroxy-2- methylazetidine-1-carbonyl]-5- methylpyrrolo[1,2-b]pyridazin- 4-yl}oxy)-2-methyl-1- benzofuran-3-carboxamide | 501 |
| Example 7.93 | 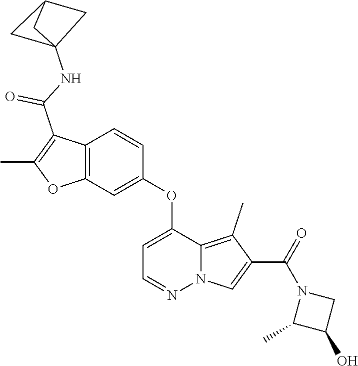 [SMILES: Cc1oc2cc(Oc3ccnn4cc(C(=O)N5C[C@@H](O)[C@@H]5C)c(C)c34)ccc2c1C(=O)NC12CC(C1)C2] | N-(bicyclo[1.1.1]pentan-1-yl)- 6-({6-[(2S,3R)-3-hydroxy-2- methylazetidine-1-carbonyl]-5- methylpyrrolo[1,2-b]pyridazin- 4-yl}oxy)-2-methyl-1- benzofuran-3-carboxamide | 501 |
| Example 7.94 | 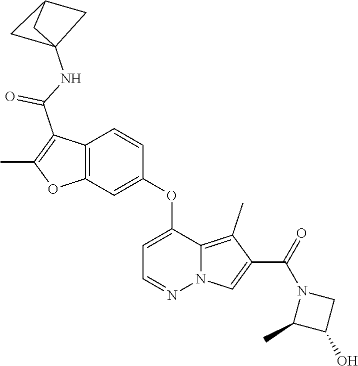 [SMILES: Cc1oc2cc(Oc3ccnn4cc(C(=O)N5C[C@H](O)[C@H]5C)c(C)c34)ccc2c1C(=O)NC12CC(C1)C2] | N-(bicyclo[1.1.1]pentan-1-yl)- 6-({6-[(2R,3S)-3-hydroxy-2- methylazetidine-1-carbonyl]-5- methylpyrrolo[1,2-b]pyridazin- 4-yl}oxy)-2-methyl-1- benzofuran-3-carboxamide | 501 |
| Example 7.95 | 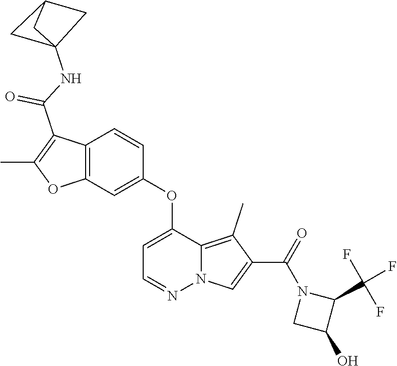 [SMILES: Cc1oc2cc(Oc3ccnn4cc(C(=O)N5C[C@H](O)[C@@H]5C(F)(F)F)c(C)c34)ccc2c1C(=O)NC12CC(C1)C2] | N-(bicyclo[1.1.1]pentan-1-yl)- 6-({6-[(2R,3S)-3-hydroxy-2- (trifluoromethyl)azetidine-1- carbonyl]-5-methylpyrrolo[1,2- b]pyridazin-4-yl}oxy)-2- methyl-1-benzofuran-3- carboxamide | 555 |
| Example 7.96 | 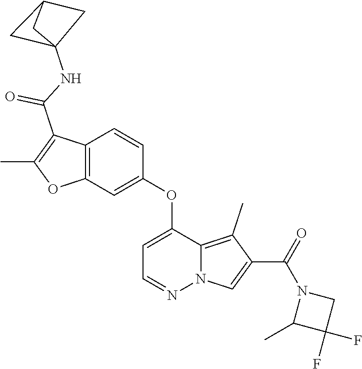 [SMILES: Cc1oc2cc(Oc3ccnn4cc(C(=O)N5CC(F)(F)C5C)c(C)c34)ccc2c1C(=O)NC12CC(C1)C2] | (rac)-N-(bicyclo[1.1.1]pentan- 1-yl)-6-{[6-(3,3-difluoro-2- methylazetidine-1-carbonyl)-5- methylpyrrolo[1,2-b]pyridazin- 4-yl]oxy}-2-methyl-1- benzofuran-3-carboxamide | 521 |
| Example 7.97 | 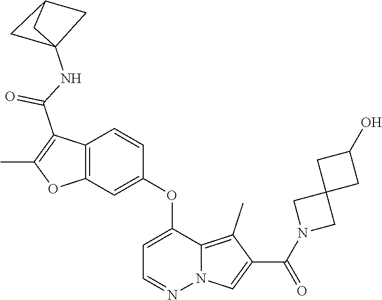 [SMILES: Cc1oc2cc(Oc3ccnn4cc(C(=O)N5CC6(CC(O)C6)C5)c(C)c34)ccc2c1C(=O)NC12CC(C1)C2] | N-(bicyclo[1.1.1]pentan-1-yl)- 6-{[6-(6-hydroxy-2- azaspiro[3.3]heptane-2- carbonyl)-5-methylpyrrolo[1,2- b]pyridazin-4-yl]oxy}-2- methyl-1-benzofuran-3- carboxamide | 527 |
| Example 7.98 | 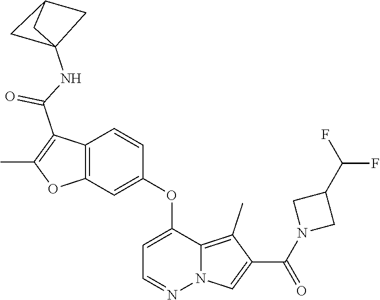 [SMILES: Cc1oc2cc(Oc3ccnn4cc(C(=O)N5CC(C(F)F)C5)c(C)c34)ccc2c1C(=O)NC12CC(C1)C2] | N-(bicyclo[1.1.1]pentan-1-yl)- 6-({6-[3- (difluoromethyl)azetidine-1- carbonyl]-5-methylpyrrolo[1,2- b]pyridazin-4-yl}oxy)-2- methyl-1-benzofuran-3- carboxamide | 521 |
| Example 7.99 | 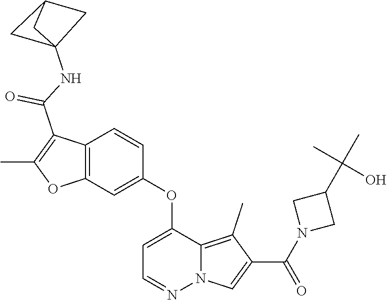 [SMILES: Cc1oc2cc(Oc3ccnn4cc(C(=O)N5CC(C(C)(C)O)C5)c(C)c34)ccc2c1C(=O)NC12CC(C1)C2] | N-(bicyclo[1.1.1]pentan-1-yl)- 6-({6-[3-(2-hydroxypropan-2- yl)azetidine-1-carbonyl]-5- methylpyrrolo[1,2-b]pyridazin- 4-yl}oxy)-2-methyl-1- benzofuran-3-carboxamide | 529 |
| Example 7.100 | 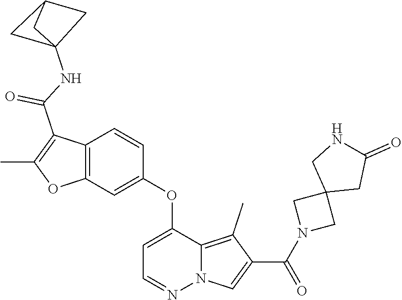 [SMILES: Cc1oc2cc(Oc3ccnn4cc(C(=O)N5CC6(CNC(=O)C6)C5)c(C)c34)ccc2c1C(=O)NC12CC(C1)C2] | N-(bicyclo[1.1.1]pentan-1-yl)- 2-methyl-6-{[5-methyl-6-(7- oxo-2,6-diazaspiro[3.4]octane- 2-carbonyl)pyrrolo[1,2- b]pyridazin-4-yl]oxy}-1- benzofuran-3-carboxamide | 540 |
| Example 7.101 | 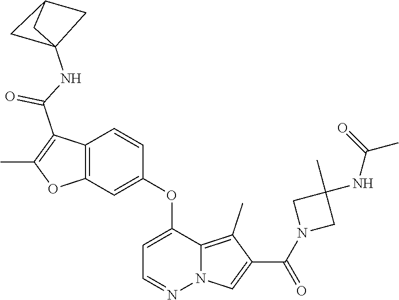 [SMILES: CC(=O)NC1(C)CN(C(=O)c2cn3nccc(Oc4ccc5c(C(=O)NC67CC(C6)C7)c(C)oc5c4)c3c2C)C1] | 6-({6-[3-(acetylamino)-3- methylazetidine-1-carbonyl]-5- methylpyrrolo[1,2-b]pyridazin- 4-yl}oxy)-N- (bicyclo[1.1.1]pentan-1-yl)-2- methyl-1-benzofuran-3- carboxamide | 542 |
| Example 7.102 | 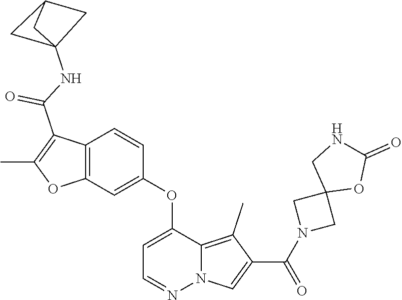 [SMILES: Cc1oc2cc(Oc3ccnn4cc(C(=O)N5CC6(CNC(=O)O6)C5)c(C)c34)ccc2c1C(=O)NC12CC(C1)C2] | N-(bicyclo[1.1.1]pentan-1-yl)- 2-methyl-6-{[5-methyl-6-(6- oxo-5-oxa-2,7- diazaspiro[3.4]octane-2- carbonyl)pyrrolo[1,2- b]pyridazin-4-yl]oxy}-1- benzofuran-3-carboxamide | 542 |
| Example 7.103 | 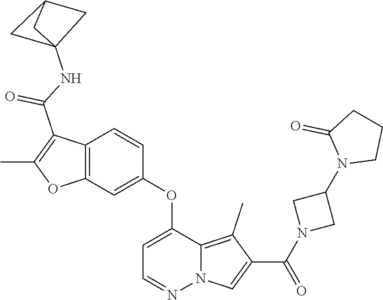 [SMILES: Cc1oc2cc(Oc3ccnn4cc(C(=O)N5CC(N6CCCC6=O)C5)c(C)c34)ccc2c1C(=O)NC12CC(C1)C2] | N-(bicyclo[1.1.1]pentan-1-yl)- 2-methyl-6-({5-methyl-6-[3-(2- oxopyrrolidin-1-yl)azetidine-1- carbonyl]pyrrolo[1,2- b]pyridazin-4-yl}oxy)-1- benzofuran-3-carboxamide | 554 |
| Example 7.104 | 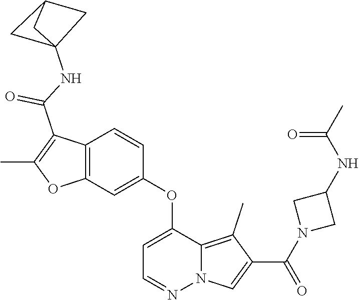 [SMILES: CC(=O)NC1CN(C(=O)c2cn3nccc(Oc4ccc5c(C(=O)NC67CC(C6)C7)c(C)oc5c4)c3c2C)C1] | 6-({6-[3- (acetylamino)azetidine-1- carbonyl]-5-methylpyrrolo[1,2- b]pyridazin-4-yl}oxy)-N- (bicyclo[1.1.1]pentan-1-yl)-2- methyl-1-benzofuran-3- carboxamide | 528 |
| Example 7.105 | 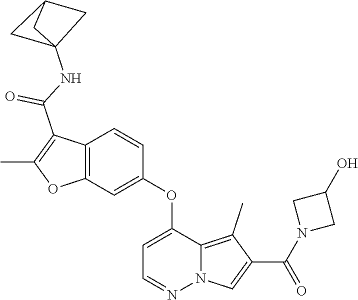 [SMILES: Cc1oc2cc(Oc3ccnn4cc(C(=O)N5CC(O)C5)c(C)c34)ccc2c1C(=O)NC12CC(C1)C2] | N-(bicyclo[1.1.1]pentan-1-yl)- 6-{[6-(3-hydroxyazetidine-1- carbonyl)-5-methylpyrrolo[1,2- b]pyridazin-4-yl]oxy}-2- methyl-1-benzofuran-3- carboxamide | 487 |
| Example 7.106 | 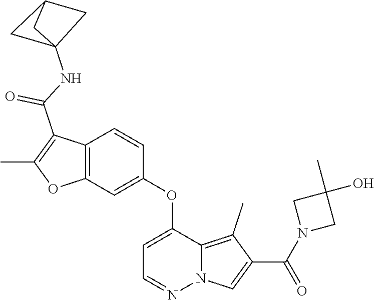 [SMILES: Cc1oc2cc(Oc3ccnn4cc(C(=O)N5CC(C)(O)C5)c(C)c34)ccc2c1C(=O)NC12CC(C1)C2] | N-(bicyclo[1.1.1]pentan-1-yl)- 6-{[6-(3-hydroxy-3- methylazetidine-1-carbonyl)-5- methylpyrrolo[1,2-b]pyridazin- 4-yl]oxy}-2-methyl-1- benzofuran-3-carboxamide | 501 |
| Example 7.107 | 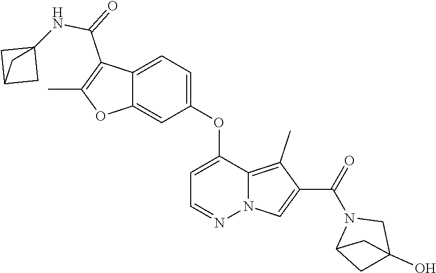 [SMILES: Cc1oc2cc(Oc3ccnn4cc(C(=O)N5CC6(O)CC5C6)c(C)c34)ccc2c1C(=O)NC12CC(C1)C2] | N-(bicyclo[1.1.1]pentan-1-yl)- 6-{[6-(4-hydroxy-2- azabicyclo[2.1.1]hexane-2- carbonyl)-5-methylpyrrolo[1,2- b]pyridazin-4-yl]oxy}-2- methyl-1-benzofuran-3- carboxamide | 513 |
| Example 7.108 | 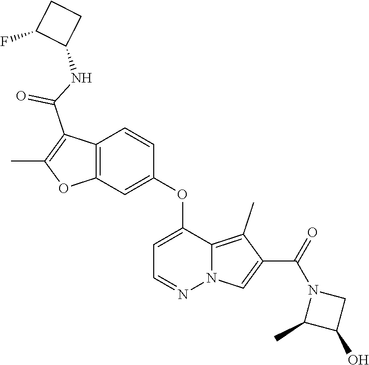 [SMILES: Cc1oc2cc(Oc3ccnn4cc(C(=O)N5C[C@@H](O)[C@H]5C)c(C)c34)ccc2c1C(=O)N[C@H]1CC[C@H]1F] | N-[(1S,2R or 1R,2S)-2- fluorocyclobutyl]-6-({6- [(2R,3R)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.109 | 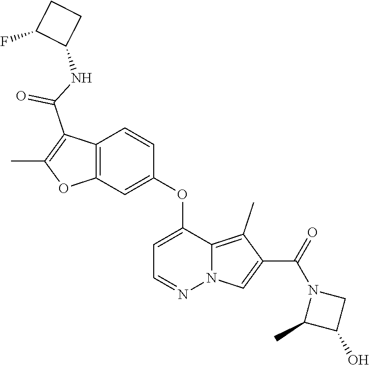 [SMILES: Cc1oc2cc(Oc3ccnn4cc(C(=O)N5C[C@H](O)[C@H]5C)c(C)c34)ccc2c1C(=O)N[C@H]1CC[C@H]1F] | N-[(1S,2R or 1R,2S)-2- fluorocyclobutyl]-6-({6- [(2R,3S)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.110 | 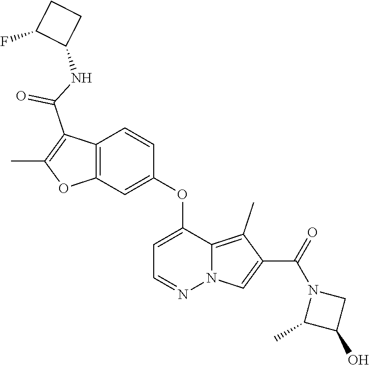 [SMILES: Cc1oc2cc(Oc3ccnn4cc(C(=O)N5C[C@@H](O)[C@@H]5C)c(C)c34)ccc2c1C(=O)N[C@H]1CC[C@H]1F] | N-[(1S,2R or 1R,2S)-2- fluorocyclobutyl]-6-({6- [(2S,3R)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.111 | 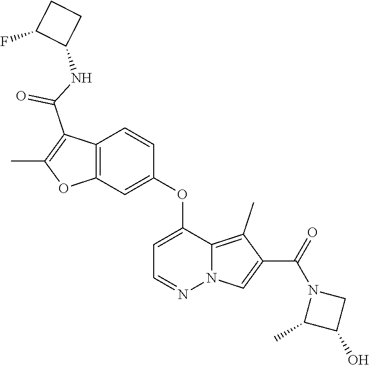 [SMILES: Cc1oc2cc(Oc3ccnn4cc(C(=O)N5C[C@H](O)[C@@H]5C)c(C)c34)ccc2c1C(=O)N[C@H]1CC[C@H]1F] | N-[(1S,2R or 1R,2S)-2- fluorocyclobutyl]-6-({6- [(2S,3S)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.112 | 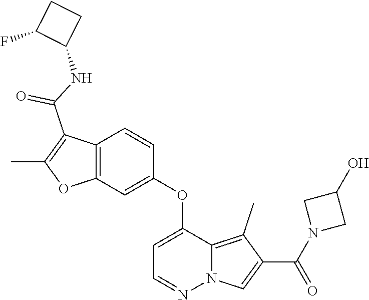 [SMILES: Cc1oc2cc(Oc3ccnn4cc(C(=O)N5CC(O)C5)c(C)c34)ccc2c1C(=O)N[C@H]1CC[C@H]1F] | N-[(1S,2R or 1R,2S)-2- fluorocyclobutyl]-6-{[6-(3- hydroxyazetidine-1-carbonyl)- 5-methylpyrrolo[1,2- b]pyridazin-4-yl]oxy}-2- methyl-1-benzofuran-3- carboxamide | 493 |
| Example 7.113 | 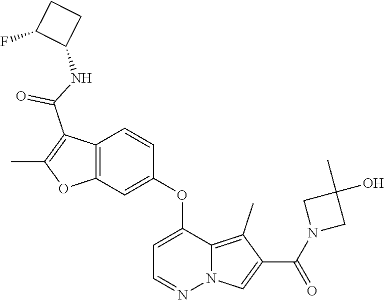 [SMILES: Cc1oc2cc(Oc3ccnn4cc(C(=O)N5CC(C)(O)C5)c(C)c34)ccc2c1C(=O)N[C@H]1CC[C@H]1F] | N-[(1S,2R or 1R,2S)-2- fluorocyclobutyl]-6-{[6-(3- hydroxy-3-methylazetidine-1- carbonyl)-5-methylpyrrolo[1,2- b]pyridazin-4-yl]oxy}-2- methyl-1-benzofuran-3- carboxamide | 507 |
| Example 7.114 | 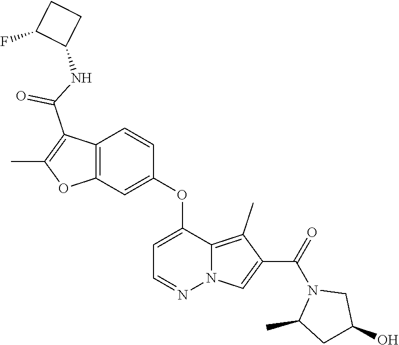 [SMILES: Cc1oc2cc(Oc3ccnn4cc(C(=O)N5C[C@@H](O)C[C@H]5C)c(C)c34)ccc2c1C(=O)N[C@H]1CC[C@H]1F] | N-[(1S,2R or 1R,2S)-2- fluorocyclobutyl]-6-({6- [(2R,4S)-4-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.115 | 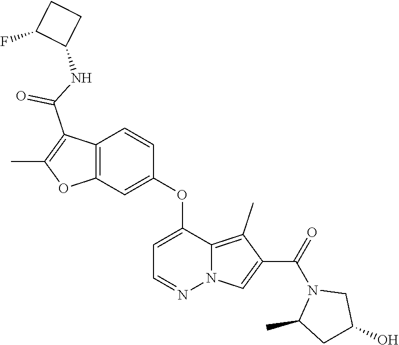 [SMILES: Cc1oc2cc(Oc3ccnn4cc(C(=O)N5C[C@H](O)C[C@H]5C)c(C)c34)ccc2c1C(=O)N[C@H]1CC[C@H]1F] | N-[(1S,2R or 1R,2S)-2- fluorocyclobutyl]-6-({6- [(2R,4R)-4-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.116 | 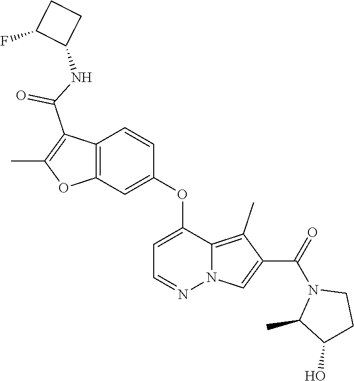 [SMILES: Cc1oc2cc(Oc3ccnn4cc(C(=O)N5CC[C@H](O)[C@H]5C)c(C)c34)ccc2c1C(=O)N[C@H]1CC[C@H]1F] | N-[(1S,2R or 1R,2S)-2- fluorocyclobutyl]-6-({6- [(2R,3S)-3-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.117 | 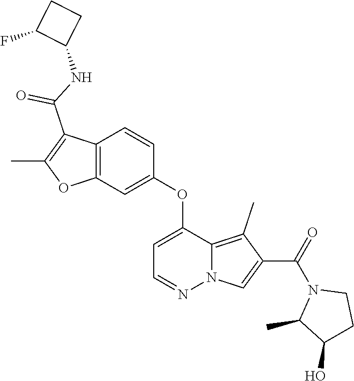 [SMILES: Cc1oc2cc(Oc3ccnn4cc(C(=O)N5CC[C@@H](O)[C@H]5C)c(C)c34)ccc2c1C(=O)N[C@H]1CC[C@H]1F] | N-[(1S,2R or 1R,2S)-2- fluorocyclobutyl]-6-({6- [(2R,3R)-3-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.118 | 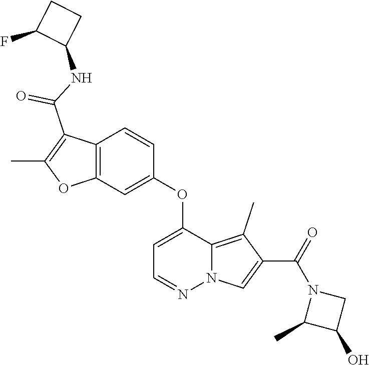 [SMILES: Cc1oc2cc(Oc3ccnn4cc(C(=O)N5C[C@@H](O)[C@H]5C)c(C)c34)ccc2c1C(=O)N[C@@H]1CC[C@@H]1F] | N-[(1R,2S or 1S,2R)-2- fluorocyclobutyl]-6-({6- [(2R,3R)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.119 | 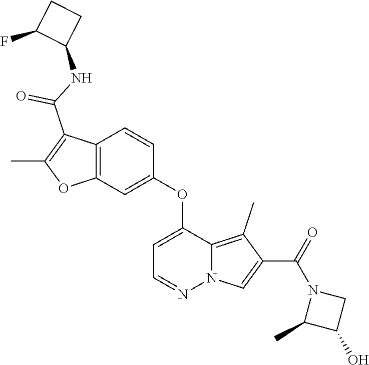 [SMILES: Cc1oc2cc(Oc3ccnn4cc(C(=O)N5C[C@H](O)[C@H]5C)c(C)c34)ccc2c1C(=O)N[C@@H]1CC[C@@H]1F] | N-[(1R,2S or 1S,2R)-2- fluorocyclobutyl]-6-({6- [(2R,3S)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.120 | 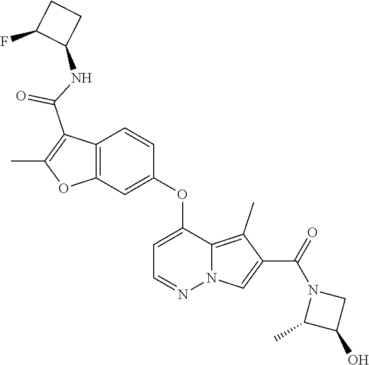 [SMILES: Cc1oc2cc(Oc3ccnn4cc(C(=O)N5C[C@@H](O)[C@@H]5C)c(C)c34)ccc2c1C(=O)N[C@@H]1CC[C@@H]1F] | N-[(1R,2S or 1S,2R)-2- fluorocyclobutyl]-6-({6- [(2S,3R)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.121 | 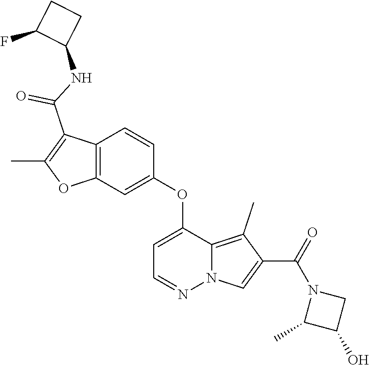 [SMILES: Cc1oc2cc(Oc3ccnn4cc(C(=O)N5C[C@H](O)[C@@H]5C)c(C)c34)ccc2c1C(=O)N[C@@H]1CC[C@@H]1F] | N-[(1R,2S or 1S,2R)-2- fluorocyclobutyl]-6-({6- [(2S,3S)-3-hydroxy-2- methylazetidine-1-carbonyl]-5- methylpyrrolo[1,2-b]pyridazin- 4-yl}oxy)-2-methyl-1- benzofuran-3-carboxamide | 507 |
| Example 7.122 | 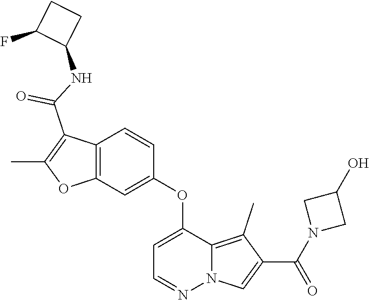 [SMILES: Cc1oc2cc(Oc3ccnn4cc(C(=O)N5CC(O)C5)c(C)c34)ccc2c1C(=O)N[C@@H]1CC[C@@H]1F] | N-[(1R,2S or 1S,2R)-2- fluorocyclobutyl]-6-{[6-(3- hydroxyazetidine-1-carbonyl)- 5-methylpyrrolo[1,2- b]pyridazin-4-yl]oxy}-2- methyl-1-benzofuran-3- carboxamide | 493 |
| Example 7.123 | 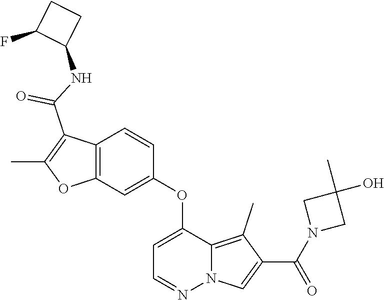 [SMILES: Cc1oc2cc(Oc3ccnn4cc(C(=O)N5CC(C)(O)C5)c(C)c34)ccc2c1C(=O)N[C@@H]1CC[C@@H]1F] | N-[(1R,2S or 1S,2R)-2- fluorocyclobutyl]-6-{[6-(3- hydroxy-3-methylazetidine-1- carbonyl)-5-methylpyrrolo[1,2- b]pyridazin-4-yl]oxy}-2- methyl-1-benzofuran-3- carboxamide | 507 |
| Example 7.124 | 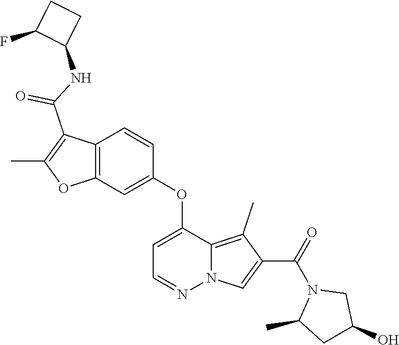 [SMILES: Cc1oc2cc(Oc3ccnn4cc(C(=O)N5C[C@@H](O)C[C@H]5C)c(C)c34)ccc2c1C(=O)N[C@@H]1CC[C@@H]1F] | N-[(1R,2S or 1S,2R)-2- fluorocyclobutyl]-6-({6- [(2R,4S)-4-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.125 | 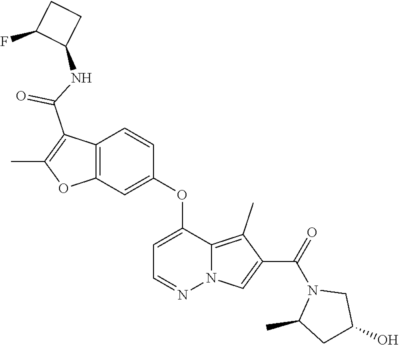 [SMILES: Cc1oc2cc(Oc3ccnn4cc(C(=O)N5C[C@H](O)C[C@H]5C)c(C)c34)ccc2c1C(=O)N[C@@H]1CC[C@@H]1F] | N-[(1R,2S or 1S,2R)-2- fluorocyclobutyl]-6-({6- [(2R,4R)-4-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.126 | 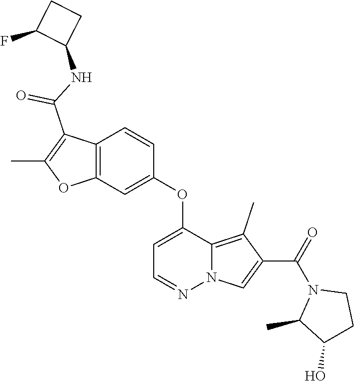 [SMILES: Cc1oc2cc(Oc3ccnn4cc(C(=O)N5CC[C@H](O)[C@H]5C)c(C)c34)ccc2c1C(=O)N[C@@H]1CC[C@@H]1F] | N-[(1R,2S or 1S,2R)-2- fluorocyclobutyl]-6-({6- [(2R,3S)-3-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.127 | 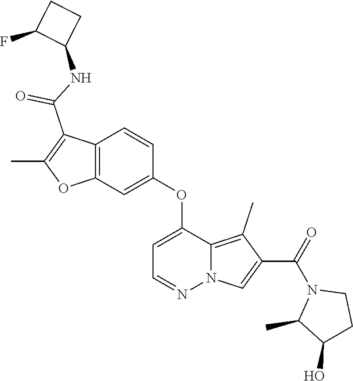 [SMILES: Cc1oc2cc(Oc3ccnn4cc(C(=O)N5CC[C@@H](O)[C@H]5C)c(C)c34)ccc2c1C(=O)N[C@@H]1CC[C@@H]1F] | N-[(1R,2S or 1S,2R)-2- fluorocyclobutyl]-6-({6- [(2R,3R)-3-hydroxy-2- methylpyrrolidine-1-carbonyl]- 5-methylpyrrolo[1,2- b]pyridazin-4-yl}oxy)-2- methyl-1-benzofuran-3- carboxamide | 521 |
| Example 7.128 | 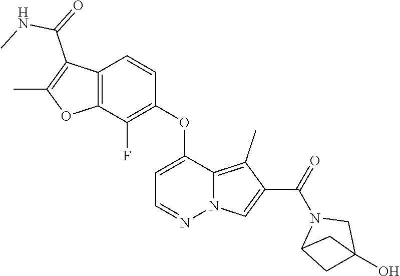 [SMILES: CNC(=O)c1c(C)oc2c(F)c(Oc3ccnn4cc(C(=O)N5CC6(O)CC5C6)c(C)c34)ccc12] | 7-fluoro-6-{[6-(4-hydroxy-2- azabicyclo[2.1.1]hexane-2- carbonyl)-5-methylpyrrolo[1,2- b]pyridazin-4-yl]oxy}-N,2- dimethyl-1-benzofuran-3- carboxamide | 479 |
| Example 7.129 | 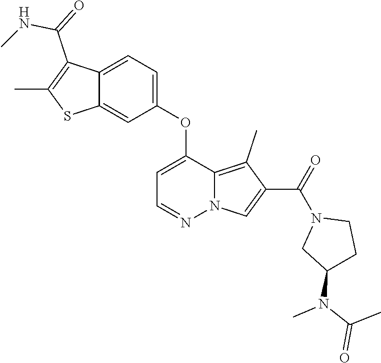 [SMILES: CNC(=O)c1c(C)sc2cc(Oc3ccnn4cc(C(=O)N5CC[C@@H](N(C)C(C)=O)C5)c(C)c34)ccc12] | (R)-N,2-dimethyl-6-((5-methyl- 6-(3-(N- methylacetamido)pyrrolidine-1- carbonyl)pyrrolo[1,2- b]pyridazin-4- yl)oxy)benzo[b]thiophene-3- carboxamide | 520 |
| Example 7.130 | 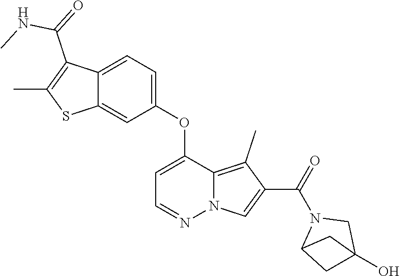 [SMILES: CNC(=O)c1c(C)sc2cc(Oc3ccnn4cc(C(=O)N5CC6(O)CC5C6)c(C)c34)ccc12] | 6-((6-(4-hydroxy-2- azabicyclo[2.1.1]hexane-2- carbonyl)-5-methylpyrrolo[1,2- b]pyridazin-4-yl)oxy)-N,2- dimethylbenzo[b]thiophene-3- carboxamide | 477 |
| Example 7.131 | 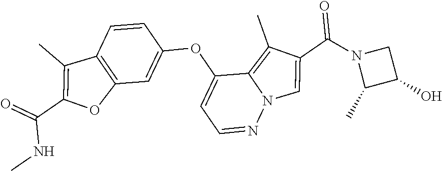 [SMILES: CNC(=O)c1oc2cc(Oc3ccnn4cc(C(=O)N5C[C@H](O)[C@@H]5C)c(C)c34)ccc2c1C] | 6-({6-[(2S,3S)-3-hydroxy-2- methylazetidine-1-carbonyl]-5- methylpyrrolo[1,2-b]pyridazin- 4-yl}oxy)-N,3-dimethyl-1- benzofuran-2-carboxamide | 449 |
| Example 7.132 | 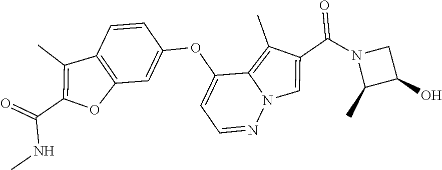 [SMILES: CNC(=O)c1oc2cc(Oc3ccnn4cc(C(=O)N5C[C@@H](O)[C@H]5C)c(C)c34)ccc2c1C] | 6-({6-[(2R,3R)-3-hydroxy-2- methylazetidine-1-carbonyl]-5- methylpyrrolo[1,2-b]pyridazin- 4-yl}oxy)-N,3-dimethyl-1- benzofuran-2-carboxamide | 449 |
| Example 7.133 | 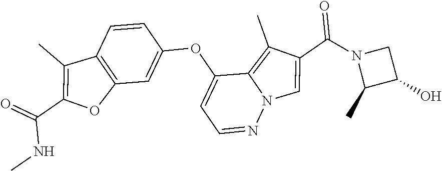 [SMILES: CNC(=O)c1oc2cc(Oc3ccnn4cc(C(=O)N5C[C@H](O)[C@H]5C)c(C)c34)ccc2c1C] | 6-({6-[(2R,3S)-3-hydroxy-2- methylazetidine-1-carbonyl]-5- methylpyrrolo[1,2-b]pyridazin- 4-yl}oxy)-N,3-dimethyl-1- benzofuran-2-carboxamide | 449 |
| Example 7.134 | 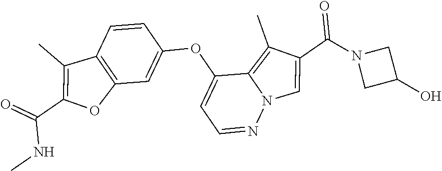 [SMILES: CNC(=O)c1oc2cc(Oc3ccnn4cc(C(=O)N5CC(O)C5)c(C)c34)ccc2c1C] | 6-{[6-(3-hydroxyazetidine-1- carbonyl)-5-methylpyrrolo[1,2- b]pyridazin-4-yl]oxy}-N,3- dimethyl-1-benzofuran-2- carboxamide | 435 |
| Example 7.135 | 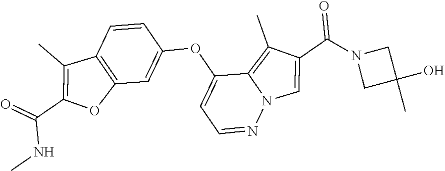 [SMILES: CNC(=O)c1oc2cc(Oc3ccnn4cc(C(=O)N5CC(C)(O)C5)c(C)c34)ccc2c1C] | 6-{[6-(3-hydroxy-3- methylazetidine-1-carbonyl)-5- methylpyrrolo[1,2-b]pyridazin- 4-yl]oxy }-N,3-dimethyl-1- benzofuran-2-carboxamide | 449 |
| Example 7.136 | 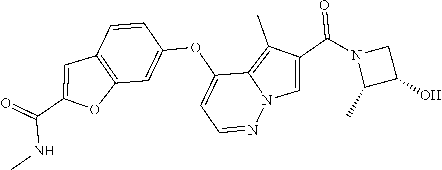 [SMILES: CNC(=O)c1cc2ccc(Oc3ccnn4cc(C(=O)N5C[C@H](O)[C@@H]5C)c(C)c34)cc2o1] | 6-({6-[(2S,3S)-3-hydroxy-2- methylazetidine-1-carbonyl]-5- methylpyrrolo[1,2-b]pyridazin- 4-yl}oxy)-N-methyl-1- benzofuran-2-carboxamide | 435 |
| Example 7.137 | 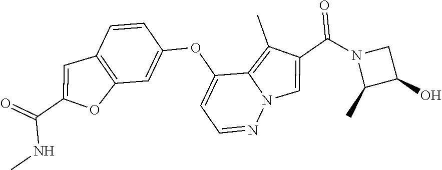 [SMILES: CNC(=O)c1cc2ccc(Oc3ccnn4cc(C(=O)N5C[C@@H](O)[C@H]5C)c(C)c34)cc2o1] | 6-({6-[(2R,3R)-3-hydroxy-2- methylazetidine-1-carbonyl]-5- methylpyrrolo[1,2-b]pyridazin- 4-yl}oxy)-N-methyl-1- benzofuran-2-carboxamide | 435 |
| Example 7.138 | 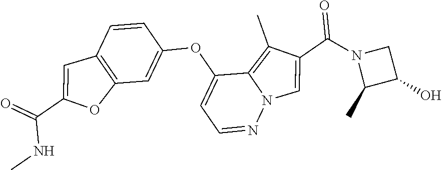 [SMILES: CNC(=O)c1cc2ccc(Oc3ccnn4cc(C(=O)N5C[C@H](O)[C@H]5C)c(C)c34)cc2o1] | 6-({6-[(2R,3S)-3-hydroxy-2- methylazetidine-1-carbonyl]-5- methylpyrrolo[1,2-b]pyridazin- 4-yl}oxy)-N-methyl-1- benzofuran-2-carboxamide | 435 |
| Example 7.139 | 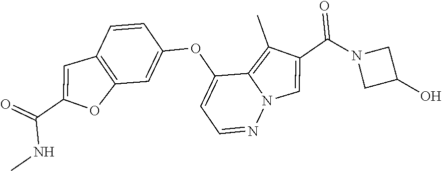 [SMILES: CNC(=O)c1cc2ccc(Oc3ccnn4cc(C(=O)N5CC(O)C5)c(C)c34)cc2o1] | 6-{[6-(3-hydroxyazetidine-1- carbonyl)-5-methylpyrrolo[1,2- b]pyridazin-4-yl]oxy}-N- methyl-1-benzofuran-2- carboxamide | 421 |
| Example 7.140 | 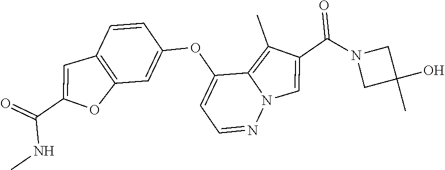 [SMILES: CNC(=O)c1cc2ccc(Oc3ccnn4cc(C(=O)N5CC(C)(O)C5)c(C)c34)cc2o1] | 6-{[6-(3-hydroxy-3- methylazetidine-1-carbonyl)-5- methylpyrrolo[1,2-b]pyridazin- 4-yl]oxy}-N-methyl-1- benzofuran-2-carboxamide | 435 |
| Example 7.141 | 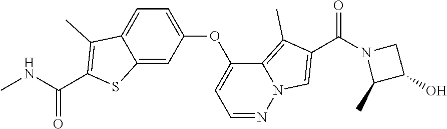 [SMILES: CNC(=O)c1sc2cc(Oc3ccnn4cc(C(=O)N5C[C@H](O)[C@H]5C)c(C)c34)ccc2c1C] | 6-({6-[(2R,3S)-3-hydroxy-2- methylazetidine-1-carbonyl]-5- methylpyrrolo[1,2-b]pyridazin- 4-yl}oxy)-N,3-dimethyl-1- benzothiophene-2-carboxamide | 465 |
| Example 7.142 | 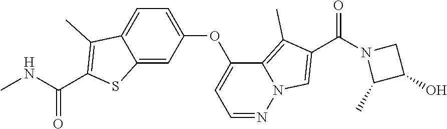 [SMILES: CNC(=O)c1sc2cc(Oc3ccnn4cc(C(=O)N5C[C@H](O)[C@@H]5C)c(C)c34)ccc2c1C] | 6-({6-[(2S,3S)-3-hydroxy-2- methylazetidine-1-carbonyl]-5- methylpyrrolo[1,2-b]pyridazin- 4-yl}oxy)-N,3-dimethyl-1- benzothiophene-2-carboxamide | 465 |
| Example 7.143 | 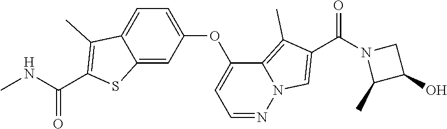 [SMILES: CNC(=O)c1sc2cc(Oc3ccnn4cc(C(=O)N5C[C@@H](O)[C@H]5C)c(C)c34)ccc2c1C] | 6-({6-[(2R,3R)-3-hydroxy-2- methylazetidine-1-carbonyl]-5- methylpyrrolo[1,2-b]pyridazin- 4-yl}oxy)-N,3-dimethyl-1- benzothiophene-2-carboxamide | 465 |
| Example 7.144 | 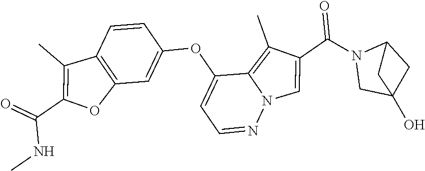 [SMILES: CNC(=O)c1oc2cc(Oc3ccnn4cc(C(=O)N5CC6(O)CC5C6)c(C)c34)ccc2c1C] | 6-{[6-(4-hydroxy-2- azabicyclo[2.1.1]hexane-2- carbonyl)-5-methylpyrrolo[1,2- b]pyridazin-4-yl]oxy}-N,3- dimethyl-1-benzothiophene-2- carboxamide | 477 |
| Example 7.145 | 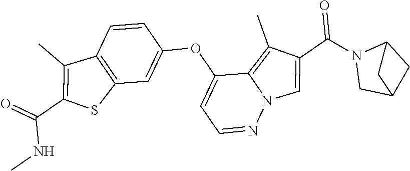 [SMILES: CNC(=O)c1sc2cc(Oc3ccnn4cc(C(=O)N5CC6CC5C6)c(C)c34)ccc2c1C] | 6-{[6-(2- azabicyclo[2.1.1]hexane-2- carbonyl)-5-methylpyrrolo[1,2- b]pyridazin-4-yl]oxy}-N,3- dimethyl-1-benzothiophene-2- carboxamide | 461 |
| Example 7.146 | 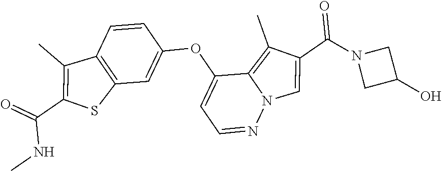 [SMILES: CNC(=O)c1sc2cc(Oc3ccnn4cc(C(=O)N5CC(O)C5)c(C)c34)ccc2c1C] | 6-{[6-(3-hydroxyazetidine-1- carbonyl)-5-methylpyrrolo[1,2- b]pyridazin-4-yl]oxy}-N,3- dimethyl-1-benzothiophene-2- carboxamide | 451 |
| Example 7.147 | 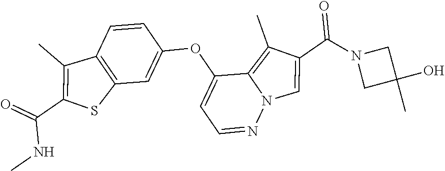 [SMILES: CNC(=O)c1sc2cc(Oc3ccnn4cc(C(=O)N5CC(C)(O)C5)c(C)c34)ccc2c1C] | 6-{[6-(3-hydroxy-3- methylazetidine-1-carbonyl)-5- methylpyrrolo[1,2-b]pyridazin- 4-yl]oxy}-N,3-dimethyl-1- benzothiophene-2-carboxamide | 465 |
| Example 7.148 | 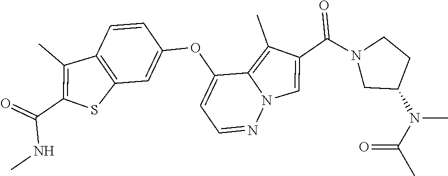 [SMILES: CNC(=O)c1sc2cc(Oc3ccnn4cc(C(=O)N5CC[C@H](N(C)C(C)=O)C5)c(C)c34)ccc2c1C] | 6-[(6-{(3S)-3- [acetyl(methyl)amino] pyrrolidine-1-carbonyl}-5- methylpyrrolo[1,2-b]pyridazin- 4-yl)oxy]-N,3-dimethyl-1- benzothiophene-2-carboxamide | 520 |
| Example 7.149 | 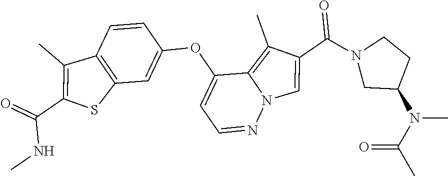 [SMILES: CNC(=O)c1sc2cc(Oc3ccnn4cc(C(=O)N5CC[C@@H](N(C)C(C)=O)C5)c(C)c34)ccc2c1C] | 6-[(6-{(3R)-3- [acetyl(methyl)amino] pyrrolidine-1-carbonyl}-5- methylpyrrolo[1,2-b]pyridazin- 4-yl)oxy]-N,3-dimethyl-1- benzothiophene-2-carboxamide | 520 |
| Example 7.150 | 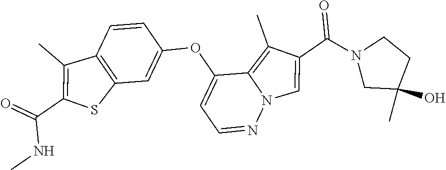 [SMILES: CNC(=O)c1sc2cc(Oc3ccnn4cc(C(=O)N5CC[C@@](C)(O)C5)c(C)c34)ccc2c1C] | 6-({6-[(3R)-3-hydroxy-3- methylpyrrolidine-1-carbonyl]- 5-methylpyrrolo[1,2- b]pyridazin-4-yl}oxy)-N,3- dimethyl-1-benzothiophene-2- carboxamide | 479 |
| Example 7.151 | 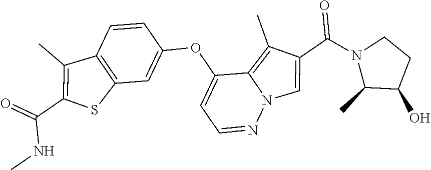 [SMILES: CNC(=O)c1sc2cc(Oc3ccnn4cc(C(=O)N5CC[C@@H](O)[C@H]5C)c(C)c34)ccc2c1C] | 6-({6-[(2R,3R)-3-hydroxy-2- methylpyrrolidine-1-carbonyl]- 5-methylpyrrolo[1,2- b]pyridazin-4-yl}oxy)-N,3- dimethyl-1-benzothiophene-2- carboxamide | 479 |
| Example 7.152 | 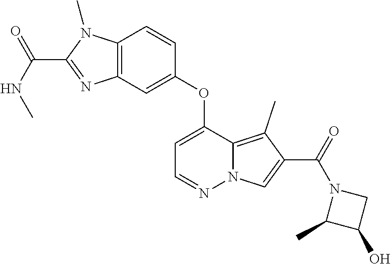 [SMILES: CNC(=O)c1nc2cc(Oc3ccnn4cc(C(=O)N5C[C@@H](O)[C@H]5C)c(C)c34)ccc2n1C] | 5-({6-[(2R,3R)-3-hydroxy-2- methylazetidine-1-carbonyl]-5- methylpyrrolo[1,2-b]pyridazin- 4-yl}oxy)-N,1-dimethyl-1H- benzimidazole-2-carboxamide | 449 |
| Example 7.2 | 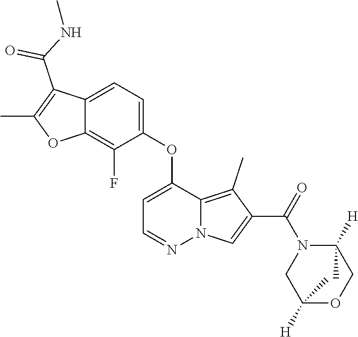 | 6-((6-((1R,4R)-2-oxa-5- azabicyclo[2.2.1]heptane-5- carbonyl)-5-methylpyrrolo[1,2- b]pyridazin-4-yl)oxy)-7-fluoro- N,2-dimethylbenzofuran-3- carboxamide | 479 |
| Example 7.3 | 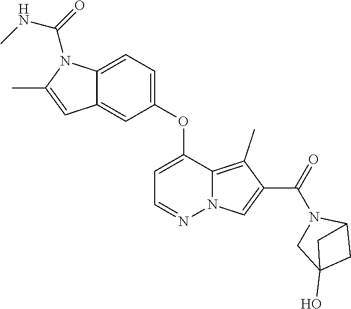 [SMILES: CNC(=O)n1c(C)cc2cc(Oc3ccnn4cc(C(=O)N5CC6(O)CC5C6)c(C)c34)ccc21] | 5-((6-(4-hydroxy-2- azabicyclo[2.1.1]hexane-2- carbonyl)-5-methylpyrrolo[1,2- b]pyridazin-4-yl)oxy)-N,2- dimethyl-1H-indole-1- carboxamide | 460 |
| Example 7.4 | 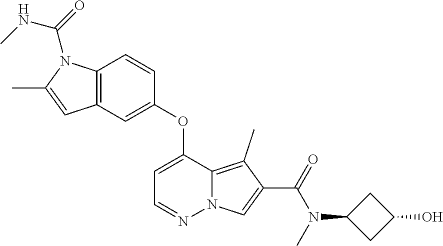 [SMILES: CNC(=O)n1c(C)cc2cc(Oc3ccnn4cc(C(=O)N(C)[C@H]5C[C@H](O)C5)c(C)c34)ccc21] | N-((1R,3R)-3- hydroxycyclobutyl)-N,5- dimethyl-4-((2-methyl-1- (methylcarbamoyl)-1H-indol-5- yl)oxy)pyrrolo[1,2- b]pyridazine-6-carboxamide | 462 |
| Example 7.5 | 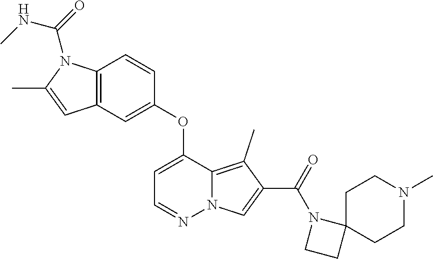 | N,2-dimethyl-5-((5-methyl-6- (7-methyl-1,7- diazaspiro[3.5]nonane-1- carbonyl)pyrrolo[1,2- b]pyridazin-4-yl)oxy)-1H- indole-1-carboxamide | 501 |
| Example 7.6 | 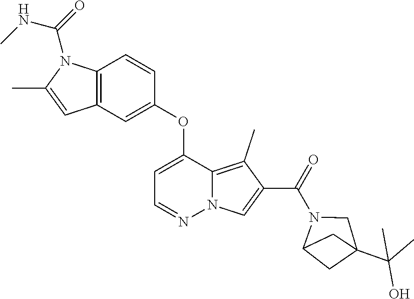 [SMILES: CNC(=O)n1c(C)cc2cc(Oc3ccnn4cc(C(=O)N5CC6(C(C)(C)O)CC5C6)c(C)c34)ccc21] | 5-((6-(4-(2-hydroxypropan-2- yl)-2-azabicyclo[2.1.1]hexane- 2-carbonyl)-5- methylpyrrolo[1,2-b]pyridazin- 4-yl)oxy)-N,2-dimethyl-1H- indole-1-carboxamide | 502 |
| Example 7.7 | 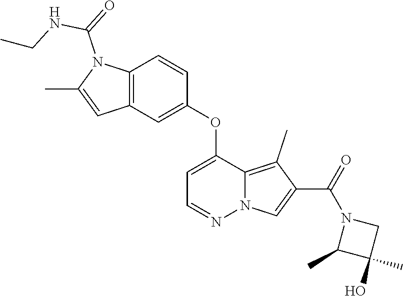 [SMILES: CCNC(=O)n1c(C)cc2cc(Oc3ccnn4cc(C(=O)N5C[C@@](C)(O)[C@H]5C)c(C)c34)ccc21] | N-ethyl-5-((6-((2R,3R)-3- hydroxy-2,3-dimethylazetidine- 1-carbonyl)-5- methylpyrrolo[1,2-b]pyridazin- 4-yl)oxy)-2-methyl-1H-indole- 1-carboxamide | 476 |
| Example 7.8 | 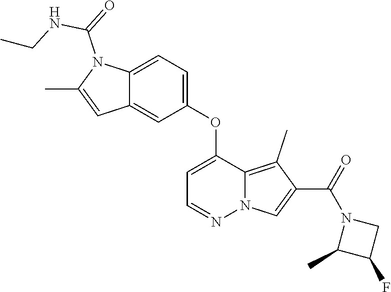 [SMILES: CCNC(=O)n1c(C)cc2cc(Oc3ccnn4cc(C(=O)N5C[C@@H](F)[C@H]5C)c(C)c34)ccc21] | N-ethyl-5-((6-((2R,3R)-3- fluoro-2-methylazetidine-1- carbonyl)-5-methylpyrrolo[1,2- b]pyridazin-4-yl)oxy)-2- methyl-1H-indole-1- carboxamide | 464 |
| Example 7.9 | 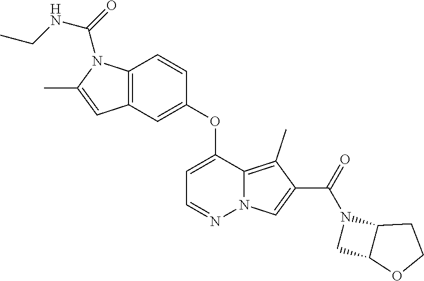 [SMILES: CCNC(=O)n1c(C)cc2cc(Oc3ccnn4cc(C(=O)N5C[C@H]6OCC[C@H]65)c(C)c34)ccc21] | 5-((6-((1R,5R)-2-oxa-6- azabicyclo[3.2.0]heptane-6- carbonyl)-5-methylpyrrolo[1,2- b]pyridazin-4-yl)oxy)-N-ethyl- 2-methyl-1H-indole-1- carboxamide | 474 |
| Example 7.10 | 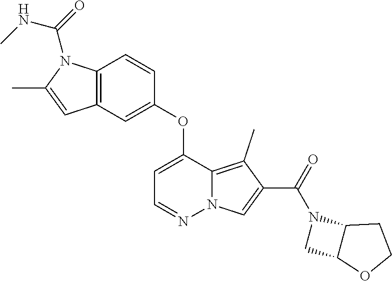 [SMILES: CNC(=O)n1c(C)cc2cc(Oc3ccnn4cc(C(=O)N5C[C@H]6OCC[C@H]65)c(C)c34)ccc21] | 5-((6-((1R,5R)-2-oxa-6- azabicyclo[3.2.0]heptane-6- carbonyl)-5-methylpyrrolo[1,2- b]pyridazin-4-yl)oxy)-N,2- dimethyl-1H-indole-1- carboxamide | 460 |
| Example 7.11 | 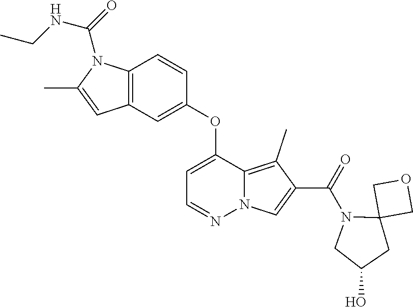 [SMILES: CCNC(=O)n1c(C)cc2cc(Oc3ccnn4cc(C(=O)N5C[C@@H](O)CC56COC6)c(C)c34)ccc21] | (S)-N-ethyl-5-((6-(7-hydroxy-2- oxa-5-azaspiro[3.4]octane-5- carbonyl)-5-methylpyrrolo[1,2- b]pyridazin-4-yl)oxy)-2- methyl-1H-indole-1- carboxamide | 503 |

### Preparation of Example 8.1: 86-((6-(4-(2-hydroxypropan-2-yl)-2-azabicyclo[2.1.1]hexane-2-carbonyl)-5-methylpyrrolo[1,2-b]pyridazin-4-yl)oxy)-N,2-dimethylbenzofuran-3-carboxamide

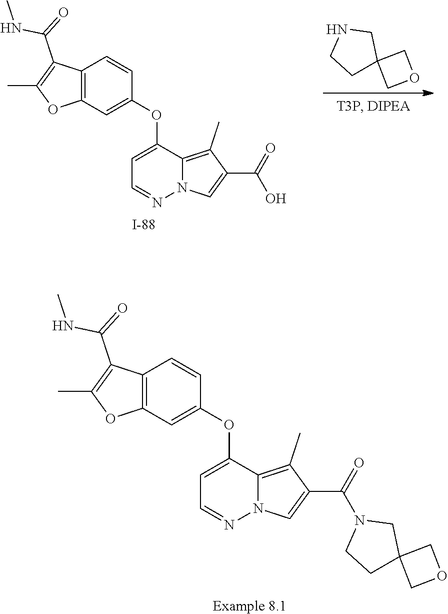

To 5-methyl-4-((2-methyl-3-(methylcarbamoyl)benzofuran-6-yl)oxy)pyrrolo[1,2-b]pyridazine-6-carboxylic acid (19.0 mg, 50.0 mol), T3P® (50 wt. % in EtOAc, 59.0 μL, 0.100 mmol) and 2-oxa-6-azaspiro[3.4]octane (22.6 mg, 0.200 mmol) in DMF (0.500 mL), DIPEA (43.5 μL, 0.400 mmol) was added. The mixture was allowed to stir at 50° C. for 16 hours. The final mixture was concentrated, dissolved in DMSO and purified by mass-directed reverse phase HPLC (MeCN/water with 0.1% TFA modifier) to afford the desired product. MS: 475 [M+1]⁺. ¹H NMR (500 MHz, DMSO-d₆) δ 8.08 (d, J=20.0 Hz, 1H), 8.00-7.96 (m, 1H), 7.95-7.92 (m, 1H), 7.86 (d, J=8.5 Hz, 1H), 7.66 (s, 1H), 7.27 (d, J=8.2 Hz, 1H), 5.64-5.61 (m, 1H), 4.63 (d, J=5.3 Hz, 1H), 4.56-4.42 (m, 3H), 3.75 (d, J=14.5 Hz, 2H), 3.56-3.49 (m, 2H), 2.83 (d, J=4.6 Hz, 3H), 2.65 (s, 3H), 2.56 (s, 3H), 2.20-2.15 (m, 2H).

TABLE 12
Utilizing a method similar to that described for the preparation of Example 8.1 and
the appropriate starting materials, the following examples were prepared:

| | | | Exact |
|---|---|---|---|
| | | | Mass |
| Compound | | | [M + |
| Number | Structure | Name | 1]⁺ |
| Example 8.2 | 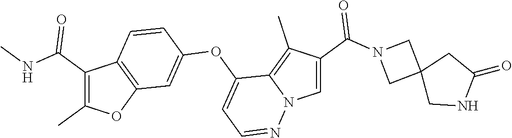 [SMILES: CNC(=O)c1c(C)oc2cc(Oc3ccnn4cc(C(=O)N5CC6(CNC(=O)C6)C5)c(C)c34)ccc12] | N,2-dimethyl-6-{[5-methyl-6- (7-oxo-2,6- diazaspiro[3.4]octane-2- carbonyl)pyrrolo[1,2- b]pyridazin-4-yl]oxy}-1- benzofuran-3-carboxamide | 488 |
| Example 8.3 | 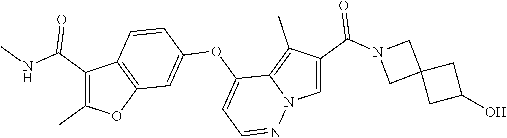 [SMILES: CNC(=O)c1c(C)oc2cc(Oc3ccnn4cc(C(=O)N5CC6(CC(O)C6)C5)c(C)c34)ccc12] | 6-{[6-(6-hydroxy-2- azaspiro[3.3]heptane-2- carbonyl)-5- methylpyrrolo[1,2- b]pyridazin-4-yl]oxy}-N,2- dimethyl-1-benzofuran-3- carboxamide | 475 |
| Example 8.4 | 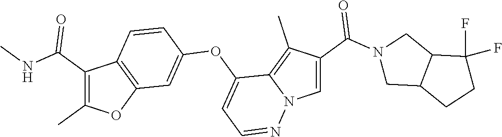 [SMILES: CNC(=O)c1c(C)oc2cc(Oc3ccnn4cc(C(=O)N5CC6CCC(F)(F)C6C5)c(C)c34)ccc12] | (rac)-6-{[6-(4,4- difluorohexahydrocyclopenta [c]pyrrole-2(1H)-carbonyl)-5- methylpyrrolo[1,2- b]pyridazin-4-yl]oxy}-N,2- dimethyl-1-benzofuran-3- carboxamide | 509 |
| Example 8.5 | 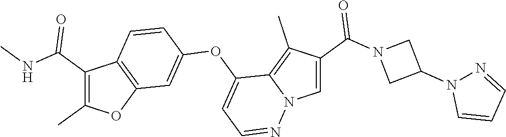 [SMILES: CNC(=O)c1c(C)oc2cc(Oc3ccnn4cc(C(=O)N5CC(n6cccn6)C5)c(C)c34)ccc12] | N,2-dimethyl-6-({5-methyl-6- [3-(1H-pyrazol-1- yl)azetidine-1- carbonyl]pyrrolo[1,2- b]pyridazin-4-yl}oxy)-1- benzofuran-3-carboxamide | 485 |
| Example 8.6 | 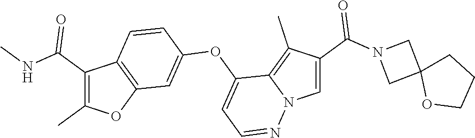 [SMILES: CNC(=O)c1c(C)oc2cc(Oc3ccnn4cc(C(=O)N5CC6(CCCO6)C5)c(C)c34)ccc12] | N,2-dimethyl-6-{[5-methyl-6- (5-oxa-2-azaspiro[3.4]octane- 2-carbonyl)pyrrolo[1,2- b]pyridazin-4-yl]oxy}-1- benzofuran-3-carboxamide | 475 |
| Example 8.7 | 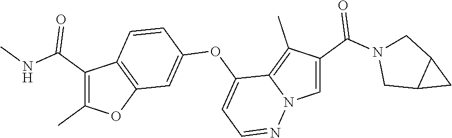 [SMILES: CNC(=O)c1c(C)oc2cc(Oc3ccnn4cc(C(=O)N5CC6CC6C5)c(C)c34)ccc12] | (rac)-6-{[6-(3- azabicyclo[3.1.0]hexane-3- carbonyl)-5- methylpyrrolo[1,2- b]pyridazin-4-yl]oxy}-N,2- dimethyl-1-benzofuran-3- carboxamide | 445 |
| Example 8.8 | 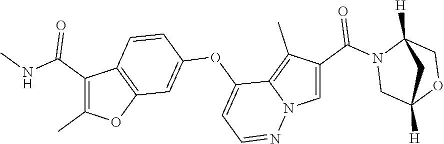 [SMILES: CNC(=O)c1c(C)oc2cc(Oc3ccnn4cc(C(=O)N5C[C@@H]6C[C@H]5CO6)c(C)c34)ccc12] | N,2-dimethyl-6-({5-methyl-6- [(1S,4S)-2-oxa-5- azabicyclo[2.2.1]heptane-5- carbonyl]pyrrolo[1,2- b]pyridazin-4-yl}oxy)-1- benzofuran-3-carboxamide | 461 |
| Example 8.9 | 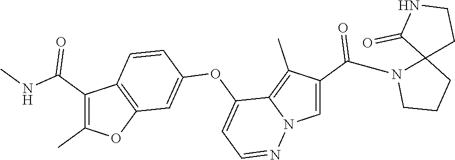 [SMILES: CNC(=O)c1c(C)oc2cc(Oc3ccnn4cc(C(=O)N5CCCC56CCNC6=O)c(C)c34)ccc12] | (rac)-N,2-dimethyl-6-{[5- methyl-6-(6-oxo-1,7- diazaspiro[4.4]nonane-1- carbonyl)pyrrolo[1,2- b]pyridazin-4-yl]oxy}-1- benzofuran-3-carboxamide | 502 |
| Example 8.10 | 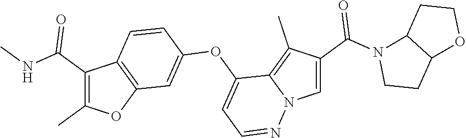 [SMILES: CNC(=O)c1c(C)oc2cc(Oc3ccnn4cc(C(=O)N5CCC6OCCC65)c(C)c34)ccc12] | (rac)-6-{[6-(hexahydro-4H- furo[3,2-b]pyrrole-4- carbonyl)-5- methylpyrrolo[1,2- b]pyridazin-4-yl]oxy}-N,2- dimethyl-1-benzofuran-3- carboxamide | 475 |
| Example 8.11 | 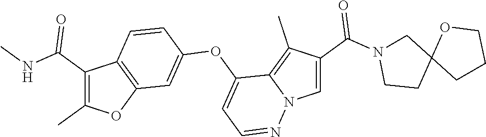 [SMILES: CNC(=O)c1c(C)oc2cc(Oc3ccnn4cc(C(=O)N5CCC6(CCCO6)C5)c(C)c34)ccc12] | (rac)-N,2-dimethyl-6-{[5- methyl-6-(1-oxa-7- azaspiro[4.4]nonane-7- carbonyl)pyrrolo[1,2- b]pyridazin-4-yl]oxy}-1- benzofuran-3-carboxamide | 489 |
| Example 8.12 | 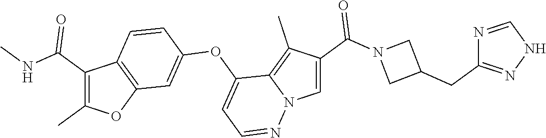 [SMILES: CNC(=O)c1c(C)oc2cc(Oc3ccnn4cc(C(=O)N5CC(Cc6nc[nH]n6)C5)c(C)c34)ccc12] | N,2-dimethyl-6-[(5-methyl-6- {3-[(1H-1,2,4-triazol-3- yl)methyl]azetidine-1- carbonyl}pyrrolo[1,2- b]pyridazin-4-yl)oxy]-1- benzofuran-3-carboxamide | 500 |
| Example 8.13 | 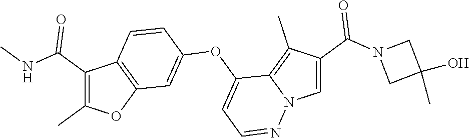 [SMILES: CNC(=O)c1c(C)oc2cc(Oc3ccnn4cc(C(=O)N5CC(C)(O)C5)c(C)c34)ccc12] | 6-{[6-(3-hydroxy-3- methylazetidine-1-carbonyl)- 5-methylpyrrolo[1,2- b]pyridazin-4-yl]oxy}-N,2- dimethyl-1-benzofuran-3- carboxamide | 449 |
| Example 8.14 | 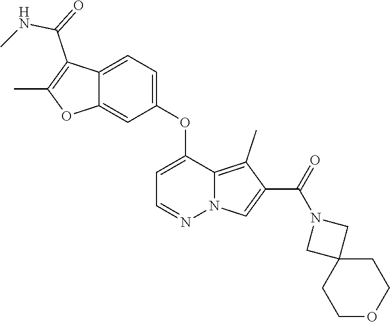 [SMILES: CNC(=O)c1c(C)oc2cc(Oc3ccnn4cc(C(=O)N5CC6(CCOCC6)C5)c(C)c34)ccc12] | N,2-dimethyl-6- {[5-methyl-6- (7-oxa-2- azaspiro[3.5]nonane-2- carbonyl)pyrrolo[1,2- b]pyridazin-4-yl]oxy}-1- benzofuran-3-carboxamide | 489 |
| Example 8.15 | 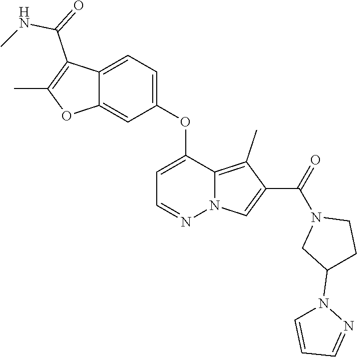 [SMILES: CNC(=O)c1c(C)oc2cc(Oc3ccnn4cc(C(=O)N5CCC(n6cccn6)C5)c(C)c34)ccc12] | (rac)-N,2-dimethyl-6-({5- methyl-6-[3-(1H-pyrazol-1- yl)pyrrolidine-1- carbonyl]pyrrolo[1,2- b]pyridazin-4-yl}oxy)-1- benzofuran-3-carboxamide | 499 |
| Example 8.16 | 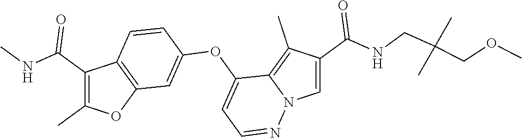 | N-(3-methoxy-2,2- dimethylpropyl)-5-methyl-4- {[2-methyl-3- (methylcarbamoyl)-1- benzofuran-6- yl]oxy}pyrrolo[1,2- b]pyridazine-6-carboxamide | 479 |
| Example 8.17 | 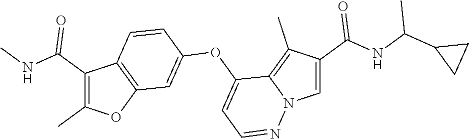 [SMILES: CNC(=O)c1c(C)oc2cc(Oc3ccnn4cc(C(=O)NC(C)C5CC5)c(C)c34)ccc12] | (rac)-N-(1-cyclopropylethyl)- 5-methyl-4-{[2-methyl-3- (methylcarbamoyl)-1- benzofuran-6- yl]oxy}pyrrolo[1,2- b]pyridazine-6-carboxamide | 447 |
| Example 8.18 | 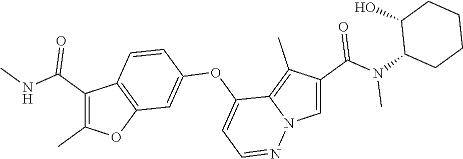 [SMILES: CNC(=O)c1c(C)oc2cc(Oc3ccnn4cc(C(=O)N(C)[C@H]5CCCC[C@H]5O)c(C)c34)ccc12] | N-[(1S,2R)-2- hydroxycyclohexyl]-N,5- dimethyl-4-{[2-methyl-3- (methylcarbamoyl)-1- benzofuran-6- yl]oxy}pyrrolo[1,2- b]pyridazine-6-carboxamide | 491 |
| Example 8.19 | 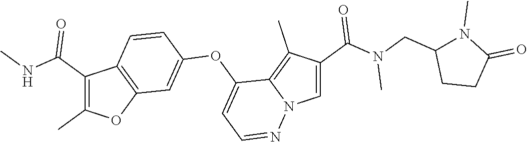 [SMILES: CNC(=O)c1c(C)oc2cc(Oc3ccnn4cc(C(=O)N(C)CC5CCC(=O)N5C)c(C)c34)ccc12] | (rac)-N,5-dimethyl-4-{[2- methyl-3-(methylcarbamoyl)- 1-benzofuran-6-yl]oxy}-N- [(1-methyl-5-oxopyrrolidin-2- yl)methyl]pyrrolo[1,2- b]pyridazine-6-carboxamide | 504 |
| Example 8.20 | 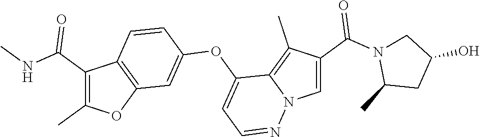 [SMILES: CNC(=O)c1c(C)oc2cc(Oc3ccnn4cc(C(=O)N5C[C@H](O)C[C@H]5C)c(C)c34)ccc12] | 6-({6-[(2R,4R)-4-hydroxy-2- methylpyrrolidine-1- carbonyl]-5- methylpyrrolo[1,2- b]pyridazin-4-yl}oxy)-N,2- dimethyl-1-benzofuran-3- carboxamide | 463 |
| Example 8.21 | 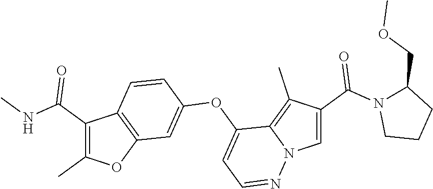 [SMILES: CNC(=O)c1c(C)oc2cc(Oc3ccnn4cc(C(=O)N5CCC[C@@H]5COC)c(C)c34)ccc12] | 6-({6-[(2R)-2- (methoxymethyl)pyrrolidine- 1-carbonyl]-5- methylpyrrolo[1,2- b]pyridazin-4-yl}oxy)-N,2- dimethyl-1-benzofuran-3- carboxamide | 477 |
| Example 8.22 | 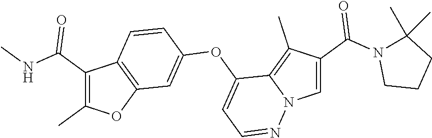 [SMILES: CNC(=O)c1c(C)oc2cc(Oc3ccnn4cc(C(=O)N5CCCC5(C)C)c(C)c34)ccc12] | 6-{[6-(2,2- dimethylpyrrolidine-1- carbonyl)-5- methylpyrrolo[1,2- b]pyridazin-4-yl]oxy}-N,2- dimethyl-1-benzofuran-3- carboxamide | 461 |
| Example 8.23 | 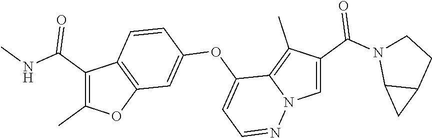 [SMILES: CNC(=O)c1c(C)oc2cc(Oc3ccnn4cc(C(=O)N5CCC6CC65)c(C)c34)ccc12] | (rac)-6-{ [6-(2- azabicyclo[3.1.0]hexane-2- carbonyl)-5- methylpyrrolo[1,2- b]pyridazin-4-yl]oxy}-N,2- dimethyl-1-benzofuran-3- carboxamide | 445 |
| Example 8.24 | 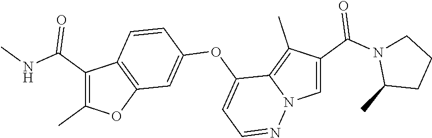 [SMILES: CNC(=O)c1c(C)oc2cc(Oc3ccnn4cc(C(=O)N5CCC[C@H]5C)c(C)c34)ccc12] | N,2-dimethyl-6-({5-methyl-6- [(2R)-2-methylpyrrolidine-1- carbonyl]pyrrolo[1,2- b]pyridazin-4-yl}oxy)-1- benzofuran-3-carboxamide | 447 |
| Example 8.25 | 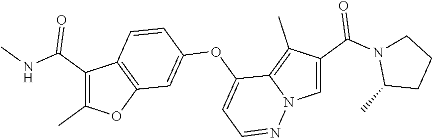 [SMILES: CNC(=O)c1c(C)oc2cc(Oc3ccnn4cc(C(=O)N5CCC[C@@H]5C)c(C)c34)ccc12] | N,2-dimethyl-6-({5-methyl-6- [(2S)-2-methylpyrrolidine-1- carbonyl]pyrrolo[1,2- b]pyridazin-4-yl}oxy)-1- benzofuran-3-carboxamide | 447 |
| Example 8.26 | 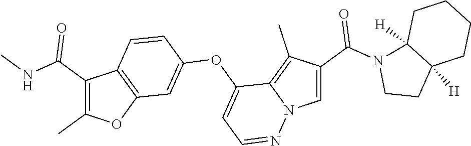 [SMILES: CNC(=O)c1c(C)oc2cc(Oc3ccnn4cc(C(=O)N5CC[C@@H]6CCCC[C@@H]65)c(C)c34)ccc12] | (cis)-N,2-dimethyl-6-((5- methyl-6-(octahydro-1H- indole-1- carbonyl)pyrrolo[1,2- b]pyridazin-4- yl)oxy)benzofuran-3- carboxamide | 487 |
| Example 8.27 | 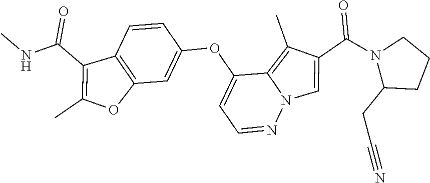 [SMILES: CNC(=O)c1c(C)oc2cc(Oc3ccnn4cc(C(=O)N5CCCC5CC#N)c(C)c34)ccc12] | (rac)-6-({6-[2- (cyanomethyl)pyrrolidine-1- carbonyl]-5- methylpyrrolo[1,2- b]pyridazin-4-yl}oxy)-N,2- dimethyl-1-benzofuran-3- carboxamide | 472 |
| Example 8.28 | 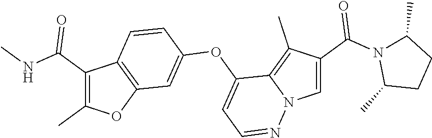 [SMILES: CNC(=O)c1c(C)oc2cc(Oc3ccnn4cc(C(=O)N5[C@H](C)CC[C@@H]5C)c(C)c34)ccc12] | (cis)-6-((6-(2,5- dimethylpyrrolidine-1- carbonyl)-5- methylpyrrolo[1,2- b]pyridazin-4-yl)oxy)-N,2- dimethylbenzofuran-3- carboxamide | 461 |
| Example 8.29 | 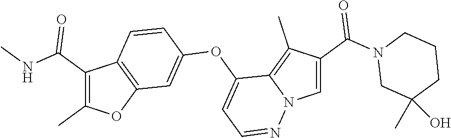 [SMILES: CNC(=O)c1c(C)oc2cc(Oc3ccnn4cc(C(=O)N5CCCC(C)(O)C5)c(C)c34)ccc12] | (rac)-6-{[6-(3-hydroxy-3- methylpiperidine-1-carbonyl)- 5-methylpyrrolo[1,2- b]pyridazin-4-yl]oxy}-N,2- dimethyl-1-benzofuran-3- carboxamide | 477 |
| Example 8.30 | 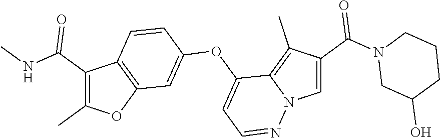 [SMILES: CNC(=O)c1c(C)oc2cc(Oc3ccnn4cc(C(=O)N5CCCC(O)C5)c(C)c34)ccc12] | (rac)-6-{ [6-(3- hydroxypiperidine-1- carbonyl)-5- methylpyrrolo[1,2- b]pyridazin-4-yl]oxy}-N,2- dimethyl-1-benzofuran-3- carboxamide | 463 |
| Example 8.31 | 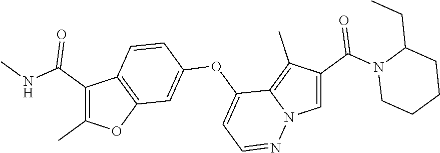 [SMILES: CCC1CCCCN1C(=O)c1cn2nccc(Oc3ccc4c(C(=O)NC)c(C)oc4c3)c2c1C] | (rac)-6-{[6-(2- ethylpiperidine-1-carbonyl)- 5-methylpyrrolo[1,2- b]pyridazin-4-yl]oxy}-N,2- dimethyl-1-benzofuran-3- carboxamide | 475 |
| Example 8.32 | 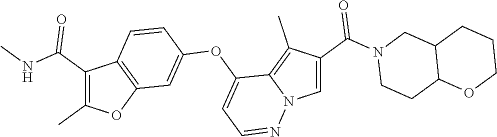 [SMILES: CNC(=O)c1c(C)oc2cc(Oc3ccnn4cc(C(=O)N5CCC6OCCCC6C5)c(C)c34)ccc12] | (cis or trans)-6-{[6- (hexahydro-2H-pyrano[3,2- c]pyridine-6(5H)-carbonyl)-5- methylpyrrolo[1,2- b]pyridazin-4-yl]oxy}-N,2- dimethyl-1-benzofuran-3- carboxamide | 503 |
| Example 8.33 | 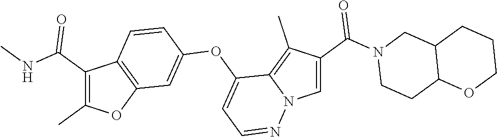 [SMILES: CNC(=O)c1c(C)oc2cc(Oc3ccnn4cc(C(=O)N5CCC6OCCCC6C5)c(C)c34)ccc12] | (trans or cis)-6-{[6- (hexahydro-2H-pyrano[3,2- c]pyridine-6(5H)-carbonyl)-5- methylpyrrolo[1,2- b]pyridazin-4-yl]oxy}-N,2- dimethyl-1-benzofuran-3- carboxamide | 503 |
| Example 8.34 | 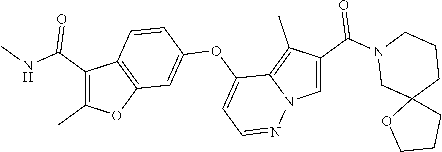 [SMILES: CNC(=O)c1c(C)oc2cc(Oc3ccnn4cc(C(=O)N5CCCC6(CCCO6)C5)c(C)c34)ccc12] | (rac)-N,2-dimethyl-6-{[5- methyl-6-(1-oxa-7- azaspiro[4.5]decane-7- carbonyl)pyrrolo[1,2- b]pyridazin-4-yl]oxy}-1- benzofuran-3-carboxamide | 503 |
| Example 8.35 | 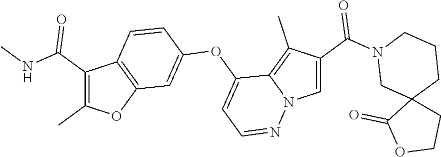 [SMILES: CNC(=O)c1c(C)oc2cc(Oc3ccnn4cc(C(=O)N5CCCC6(CCOC6=O)C5)c(C)c34)ccc12] | (rac)-N,2-dimethyl-6-{[5- methyl-6-(1-oxo-2-oxa-7- azaspiro[4.5]decane-7- carbonyl)pyrrolo[1,2- b]pyridazin-4-yl]oxy}-1- benzofuran-3-carboxamide | 517 |
| Example 8.36 | 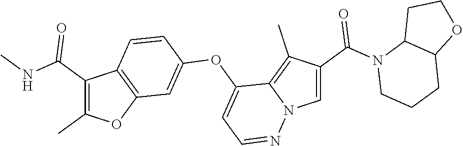 [SMILES: CNC(=O)c1c(C)oc2cc(Oc3ccnn4cc(C(=O)N5CCCC6OCCC65)c(C)c34)ccc12] | (rac)-6-{[6- (hexahydrofuro[3,2- b]pyridine-4(2H)-carbonyl)- 5-methylpyrrolo[1,2- b]pyridazin-4-yl]oxy}-N,2- dimethyl-1-benzofuran-3- carboxamide | 489 |
| Example 8.37 | 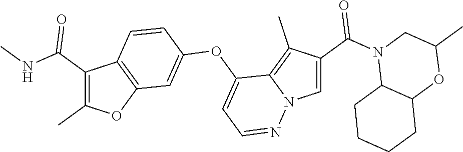 | (rac)-N,2-dimethyl-6-{[5- methyl-6-(2- methyloctahydro-4H-1,4- benzoxazine-4- carbonyl)pyrrolo[1,2- b]pyridazin-4-yl]oxy}-1- benzofuran-3-carboxamide | 517 |
| Example 8.38 | 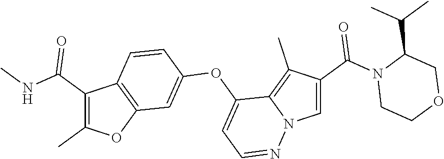 [SMILES: CNC(=O)c1c(C)oc2cc(Oc3ccnn4cc(C(=O)N5CCOC[C@@H]5C(C)C)c(C)c34)ccc12] | N,2-dimethyl-6-({5-methyl-6- [(3S)-3-(propan-2- yl)morpholine-4- carbonyl]pyrrolo[1,2- b]pyridazin-4-yl}oxy)-1- benzofuran-3-carboxamide | 491 |
| Example 8.39 | 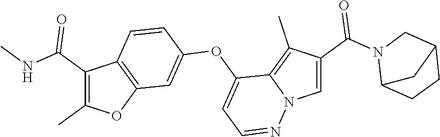 [SMILES: CNC(=O)c1c(C)oc2cc(Oc3ccnn4cc(C(=O)N5CC6CCC5C6)c(C)c34)ccc12] | (rac)-6-{[6-(2- azabicyclo[2.2.1]heptane-2- carbonyl)-5- methylpyrrolo[1,2- b]pyridazin-4-yl]oxy}-N,2- dimethyl-1-benzofuran-3- carboxamide | 459 |
| Example 8.40 | 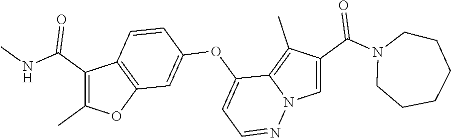 [SMILES: CNC(=O)c1c(C)oc2cc(Oc3ccnn4cc(C(=O)N5CCCCCC5)c(C)c34)ccc12] | 6-{[6-(azepane-1-carbonyl)-5- methylpyrrolo[1,2- b]pyridazin-4-yl]oxy}-N,2- dimethyl-1-benzofuran-3- carboxamide | 461 |
| Example 8.41 | 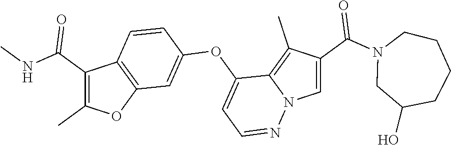 [SMILES: CNC(=O)c1c(C)oc2cc(Oc3ccnn4cc(C(=O)N5CCCCC(O)C5)c(C)c34)ccc12] | (rac)-6-{[6-(3- hydroxyazepane-1-carbonyl)- 5-methylpyrrolo[1,2- b]pyridazin-4-yl]oxy}-N,2- dimethyl-1-benzofuran-3- carboxamide | 477 |
| Example 8.42 | 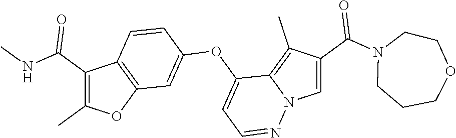 [SMILES: CNC(=O)c1c(C)oc2cc(Oc3ccnn4cc(C(=O)N5CCCOCC5)c(C)c34)ccc12] | N,2-dimethyl-6-{[5-methyl-6- (1,4-oxazepane-4- carbonyl)pyrrolo[1,2- b]pyridazin-4-yl]oxy}-1- benzofuran-3-carboxamide | 463 |
| Example 8.43 | 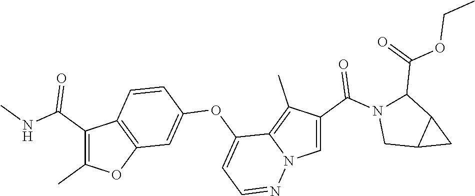 [SMILES: CCOC(=O)C1C2CC2CN1C(=O)c1cn2nccc(Oc3ccc4c(C(=O)NC)c(C)oc4c3)c2c1C] | (rac)-ethyl 3-(5-methyl-4-{[2- methyl-3-(methylcarbamoyl)- 1-benzofuran-6- yl]oxy}pyrrolo[1,2- b]pyridazine-6-carbonyl)-3- azabicyclo[3.1.0]hexane-2- carboxylate | 517 |
| Example 8.44 | 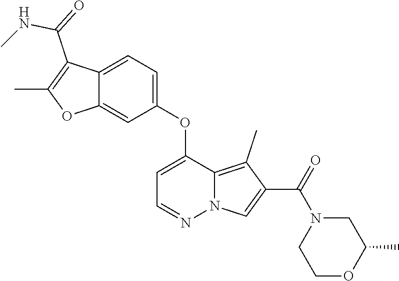 | N,2-dimethyl-6-({5-methyl-6- [(2S)-2-methylmorpholine-4- carbonyl]pyrrolo[1,2- b]pyridazin-4-yl}oxy)-1- benzofuran-3-carboxamide | 463 |
| Example 8.45 | 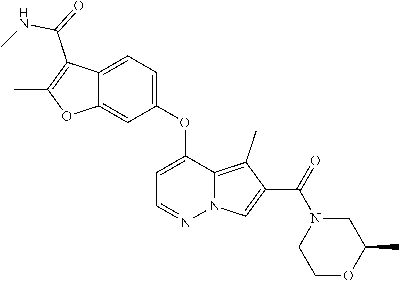 | N,2-dimethyl-6-({5-methyl-6- [(2R)-2-methylmorpholine-4- carbonyl]pyrrolo[1,2- b]pyridazin-4-yl}oxy)-1- benzofuran-3-carboxamide | 463 |
| Example 8.46 | 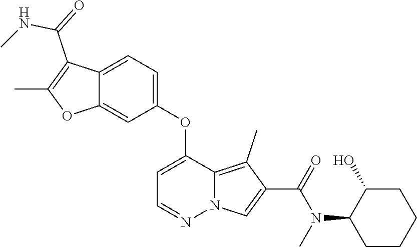 [SMILES: CNC(=O)c1c(C)oc2cc(Oc3ccnn4cc(C(=O)N(C)[C@@H]5CCCC[C@H]5O)c(C)c34)ccc12] | (trans)-N-(2- hydroxycyclohexyl)-N,5- dimethyl-4-((2-methyl-3- (methylcarbamoyl)benzofuran- 6-yl)oxy)pyrrolo[1,2- b]pyridazine-6-carboxamide | 491 |
| Example 8.47 | 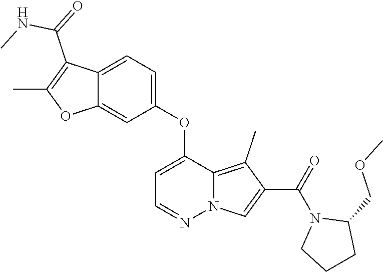 | 6-({6-[(2S)-2- (methoxymethyl)pyrrolidine- 1-carbonyl]-5- methylpyrrolo[1,2- b]pyridazin-4-yl}oxy)-N,2- dimethyl-1-benzofuran-3- carboxamide | 477 |
| Example 8.48 | 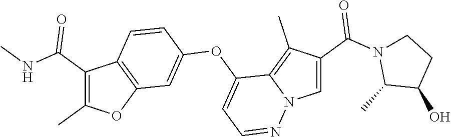 [SMILES: CNC(=O)c1c(C)oc2cc(Oc3ccnn4cc(C(=O)N5CC[C@@H](O)[C@@H]5C)c(C)c34)ccc12] | 6-({6-[(2S,3R)-3-hydroxy-2- methylpyrrolidine-1- carbonyl]-5- methylpyrrolo[1,2- b]pyridazin-4-yl}oxy)-N,2- dimethyl-1-benzofuran-3- carboxamide | 463 |
| Example 8.49 | 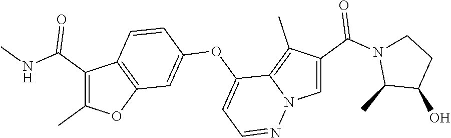 [SMILES: CNC(=O)c1c(C)oc2cc(Oc3ccnn4cc(C(=O)N5CC[C@@H](O)[C@H]5C)c(C)c34)ccc12] | 6-({6-[(2R,3R)-3-hydroxy-2- methylpyrrolidine-1- carbonyl]-5- methylpyrrolo[1,2- b]pyridazin-4-yl}oxy)-N,2- dimethyl-1-benzofuran-3- carboxamide | 463 |
| Example 8.50 | 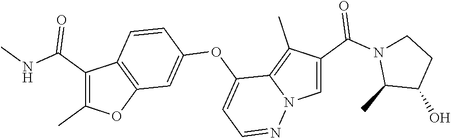 [SMILES: CNC(=O)c1c(C)oc2cc(Oc3ccnn4cc(C(=O)N5CC[C@H](O)[C@H]5C)c(C)c34)ccc12] | 6-({6-[(2R,3S)-3-hydroxy-2- methylpyrrolidine-1- carbonyl]-5- methylpyrrolo[1,2- b]pyridazin-4-yl}oxy)-N,2- dimethyl-1-benzofuran-3- carboxamide | 463 |
| Example 8.51 | 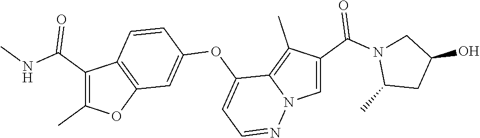 [SMILES: CNC(=O)c1c(C)oc2cc(Oc3ccnn4cc(C(=O)N5C[C@@H](O)C[C@@H]5C)c(C)c34)ccc12] | 6-({6-[(2,4S)-4-hydroxy-2- methylpyrrolidine-1- carbonyl]-5- methylpyrrolo[1,2- b]pyridazin-4-yl}oxy)-N,2- dimethyl-1-benzofuran-3- carboxamide | 463 |
| Example 8.52 | 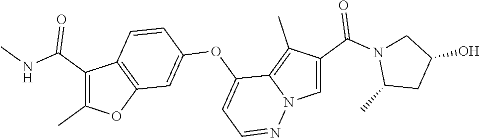 [SMILES: CNC(=O)c1c(C)oc2cc(Oc3ccnn4cc(C(=O)N5C[C@H](O)C[C@@H]5C)c(C)c34)ccc12] | 6-({6-[(2S,4R)-4-hydroxy-2- methylpyrrolidine-1- carbonyl]-5- methylpyrrolo[1,2- b] pyridazin-4-yl}oxy)-N,2- dimethyl-1-benzofuran-3- carboxamide | 463 |
| Example 8.53 | 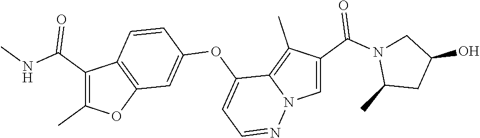 [SMILES: CNC(=O)c1c(C)oc2cc(Oc3ccnn4cc(C(=O)N5C[C@@H](O)C[C@H]5C)c(C)c34)ccc12] | 6-({6-[(2R,4S)-4-hydroxy-2- methylpyrrolidine-1- carbonyl]-5- methylpyrrolo[1,2- b]pyridazin-4-yl}oxy)-N,2- dimethyl-1-benzofuran-3- carboxamide | 463 |
| Example 8.54 | 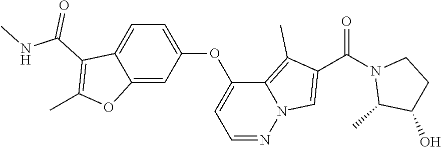 [SMILES: CNC(=O)c1c(C)oc2cc(Oc3ccnn4cc(C(=O)N5CC[C@H](O)[C@@H]5C)c(C)c34)ccc12] | 6-({6-[(2S,3S)-3-hydroxy-2- methylpyrrolidine-1- carbonyl]5- methylpyrrolo[1,2- b]pyridazin-4-yl}oxy)-N,2- dimethyl-1-benzofuran-3- carboxamide | 463 |
| Example 8.55 | 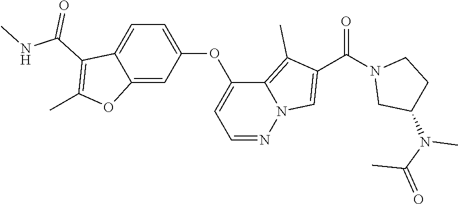 | 6-[(6-{(3S)-3- [acetyl(methyl)amino] pyrrolidine-1-carbonyl}-5- methylpyrrolo[1,2- b]pyridazin-4-yl)oxy]-N,2- dimethyl-1-benzofuran-3- carboxamide | 504 |
| Example 8.56 | 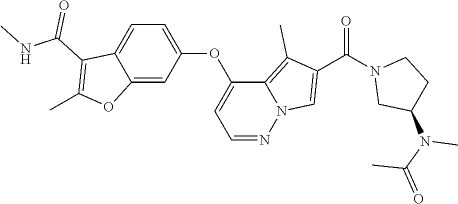 | 6-[(6-{(3R)-3- [acetyl(methyl)amino] pyrrolidine-1-carbonyl}-5- methylpyrrolo[1,2- b]pyridazin-4-yl)oxy]-N,2- dimethyl-1-benzofuran-3- carboxamide | 504 |
| Example 8.57 | 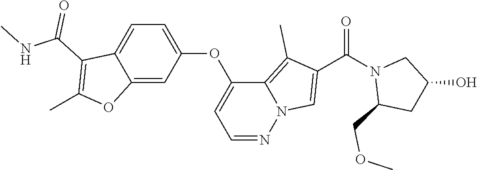 [SMILES: CNC(=O)c1c(C)oc2cc(Oc3ccnn4cc(C(=O)N5C[C@H](O)C[C@H]5COC)c(C)c34)ccc12] | 6-({6-[(2S,4R)-4-hydroxy-2- (methoxymethyl)pyrrolidine- 1-carbonyl]-5- methylpyrrolo[1,2- b]pyridazin-4-yl}oxy)-N,2- dimethyl-1-benzofuran-3- carboxamide | 493 |
| Example 8.58 | 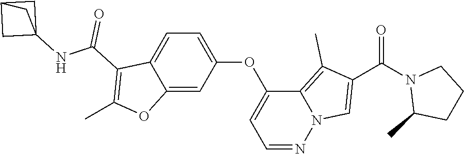 [SMILES: Cc1oc2cc(Oc3ccnn4cc(C(=O)N5CCC[C@H]5C)c(C)c34)ccc2c1C(=O)NC12CC(C1)C2] | N-(bicyclo[1.1.1]pentan-1- yl)-2-methyl-6-({5-methyl-6- [(2R)-2-methylpyrrolidine-1- carbonyl]pyrrolo[1,2- b]pyridazin-4-yl}oxy)-1- benzofuran-3-carboxamide | 499 |
| Example 8.59 | 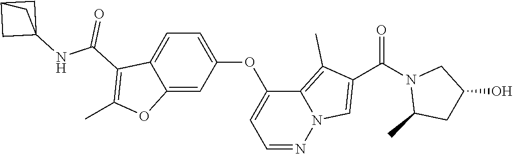 [SMILES: Cc1oc2cc(Oc3ccnn4cc(C(=O)N5C[C@H](O)C[C@H]5C)c(C)c34)ccc2c1C(=O)NC12CC(C1)C2] | N-(bicyclo[1.1.1]pentan-1- yl)-6-({6-[(2R,4R)-4- hydroxy-2-methylpyrrolidine- 1-carbonyl]-5- methylpyrrolo[1,2- b]pyridazin-4-yl}oxy)-2- methyl-1-benzofuran-3- carboxamide | 515 |
| Example 8.60 | 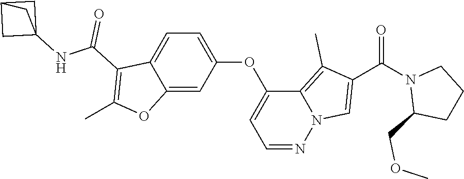 [SMILES: COC[C@@H]1CCCN1C(=O)c1cn2nccc(Oc3ccc4c(C(=O)NC56CC(C5)C6)c(C)oc4c3)c2c1C] | N-(bicyclo[1.1.1]pentan-1- yl)-6-({6-[(2S)-2- (methoxymethyl)pyrrolidine- 1-carbonyl]-5- methylpyrrolo[1,2- b]pyridazin-4-yl}oxy)-2- methyl-1-benzofuran-3- carboxamide | 529 |

### Preparation of Example 9.1A & 9.11B: Trans. N-(3,3-difluoro-2-hydroxycyclohexyl)-5-methyl-4-((2-methyl-3-(methylcarbamoyl)benzofuran-6-yl)oxy)pyrrolo[1,2-b]pyridazine-6-carboxamide)

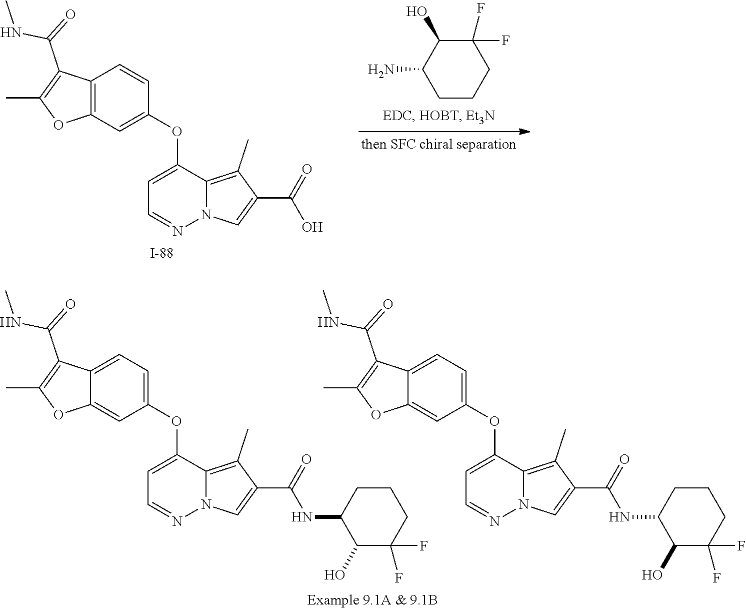

A mixture of 5-methyl-4-((2-methyl-3-(methylcarbamoyl)benzofuran-6-yl)oxy)pyrrolo[1,2-b]pyridazine-6-carboxylic acid (50.0 mg, 0.130 mmol), (trans)-6-amino-2,2-difluorocyclohexan-1-ol (19.9 mg, 0.130 mmol), HOBT (60.6 mg, 0.400 mmol), EDC (76.0 mg, 0.400 mmol), TEA (90.0 μL, 0.660 mmol) in DMF (0.500 mL) was stirred at 25° C. for 16 hours. The mixture was filtered, and the filtrate was purified by mass-directed reverse phase HPLC (MeCN/water with 0.1% NH₄OH modifier) to afford the racemate mixture of the desired product. The mixture of the two stereoisomers was purified by chiral SFC (AD-3 column, Mobile phase A: CO₂, B: 40% ethanol with 0.05% DEA) to afford isomer Example 9.1A (faster eluting) and Example 9.1B (slower eluting).

Isomer Example 9.1A: MS: 513 [M+1]⁺. ¹H NMR (400 MHz, DMSO-d₆) δ 8.29 (s, 1H), 8.01-7.96 (m, 2H), 7.94 (d, J=5.2 Hz, 1H), 7.85 (d, J=8.8 Hz, 1H), 7.66 (d, J=2.4 Hz, 1H), 7.25 (dd, J=8.4, 2.0 Hz, 1H), 5.62 (d, J=5.2 Hz, 1H), 5.52 (d, J=6.4 Hz, 1H), 3.95-3.84 (m, 1H), 3.75-3.63 (m, 1H), 2.82 (d, J=4.4 Hz, 3H), 2.71 (s, 3H), 2.64 (s, 3H), 2.12-2.03 (m, 1H), 1.93-1.82 (m, 1H), 1.76-1.64 (m, 2H), 1.45-1.37 (m, 2H).

Isomer Example 9.1B: MS: 513 [M+1]⁺. ¹H NMR (400 MHz, DMSO-d₆) δ 8.29 (s, 1H), 8.01-7.96 (m, 2H), 7.94 (d, J=5.2 Hz, 1H), 7.85 (d, J=8.8 Hz, 1H), 7.66 (d, J=2.4 Hz, 1H), 7.25 (dd, J=8.4, 2.0 Hz, 1H), 5.62 (d, J=5.2 Hz, 1H), 5.52 (d, J=6.4 Hz, 1H), 3.95-3.84 (m, 1H), 3.75-3.63 (m, 1H), 2.82 (d, J=4.4 Hz, 3H), 2.71 (s, 3H), 2.64 (s, 3H), 2.12-2.03 (m, 1H), 1.93-1.82 (m, 1H), 1.76-1.64 (m, 2H), 1.45-1.37 (m, 2H).

TABLE 13
Utilizing a method similar to that described for the preparation of Example 9.1 and
the appropriate starting materials, the following examples were prepared:

| Compound | | | Exact Mass |
|---|---|---|---|
| Number | Structure | Name | [M + 1]⁺ |
| Example 9.2A | 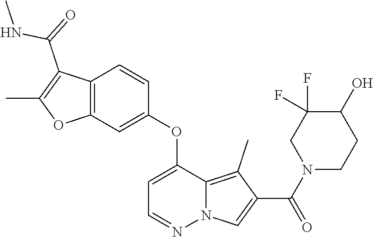 [SMILES: CNC(=O)c1c(C)oc2cc(Oc3ccnn4cc(C(=O)N5CCC(O)C(F)(F)C5)c(C)c34)ccc12] | (R or S)-6-{[6-(3,3-difluoro-4- hydroxypiperidine-1-carbonyl)-5- methylpyrrolo[1,2-b]pyridazin-4- y]]oxy}-N,2-dimethyl-1-benzofuran- 3-carboxamide | 499 |
| Example 9.2B | 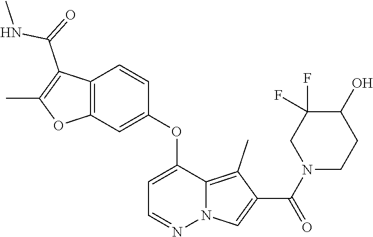 [SMILES: CNC(=O)c1c(C)oc2cc(Oc3ccnn4cc(C(=O)N5CCC(O)C(F)(F)C5)c(C)c34)ccc12] | (S or R)-6-{ [6-(3,3-difluoro-4- hydroxypiperidine-1-carbonyl)-5- methylpyrrolo[1,2-b]pyridazin-4- yl]oxy}-N,2-dimethyl-1-benzofuran- 3-carboxamide | 499 |
| Example 9.3A | 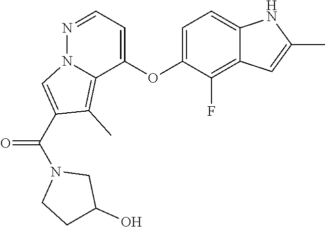 [SMILES: Cc1cc2c(F)c(Oc3ccnn4cc(C(=O)N5CCC(O)C5)c(C)c34)ccc2[nH]1] | (R or S)-{4-[(4-fluoro-2-methyl-1H- indol-5-yl)oxy]-5- methylpyrrolo[1,2-b]pyridazin-6- yl}(3-hydroxypyrrolidin-1- yl)methanone | 409 |
| Example 9.3B | 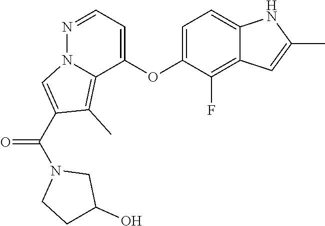 [SMILES: Cc1cc2c(F)c(Oc3ccnn4cc(C(=O)N5CCC(O)C5)c(C)c34)ccc2[nH]1] | (S or R)-{4-[(4-fluoro-2-methyl-1H- indol-5-yl)oxy]-5- methylpyrrolo[1,2-b]pyridazin-6- yl}(3-hydroxypyrrolidin-1- yl)methanone | 409 |

### Preparation of Example 10.1A & 10.1B: 6-((6-((2R,3R or 2S,3S)-3-cyano-2-methylazetidine-1-carbonyl)-5-methylpyrrolo[1,2-b]pyridazin-4-yl)oxy)-N,2-dimethylbenzofuran-3-carboxamide

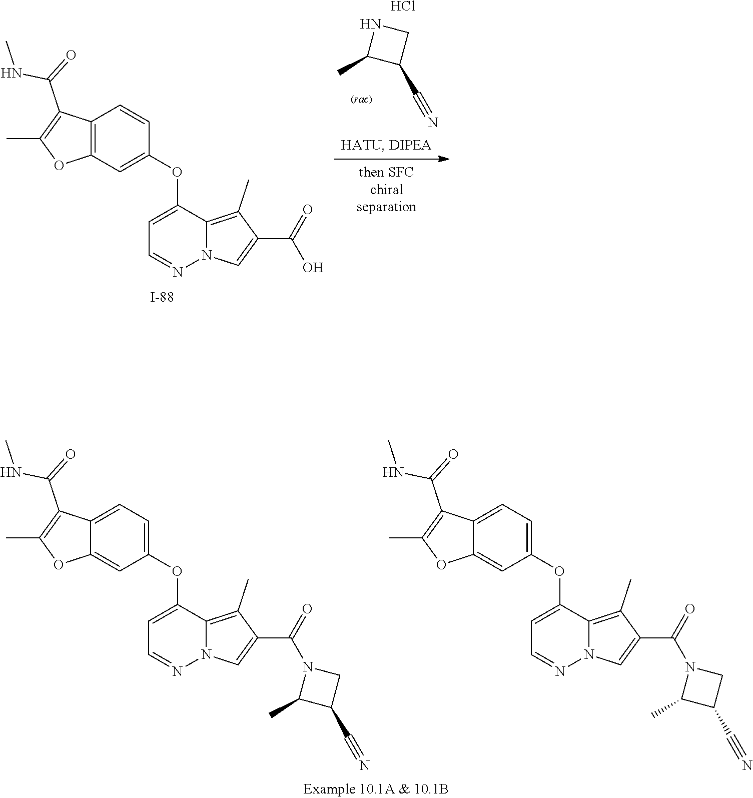

To 5-methyl-4-((2-methyl-3-(methylcarbamoyl)benzofuran-6-yl)oxy)pyrrolo[1,2-b]pyridazine-6-carboxylic acid (76.0 mg, 0.200 mmol), (cis)-2-methylazetidine-3-carbonitrile, HCl (53.0 mg, 0.400 mmol), and HATU (152 mg, 0.400 mmol) in DMF (2.00 mL), DIPEA (174 L, 1.00 mmol) was added. The reaction mixture was allowed to stir overnight at 50° C. The mixture was filtered and purified by mass triggered reverse phase HPLC (MeCN/water with 0.1% NH₄OH modifier) to afford the title compound.

The mixture of the two stereoisomers was purified by chiral SFC (CCO F4, 21×250 mm, 5 μm column, Mobile phase A: CO₂, B: 45% 1:1 MeOH/CH₃CN with 0.1% NH₄OH) to afford isomer Example 10.1A (faster eluting) and Example 10.1B (slower eluting).

Isomer Example 10.1A: MS: 458 [M+1]⁺. ¹H NMR (500 MHz, DMSO-d₆) δ 8.13 (s, 1H), 8.00-7.93 (m, 2H), 7.86 (d, J=8.5 Hz, 1H), 7.67 (d, J=1.9 Hz, 1H), 7.27 (d, J=10.5 Hz, 1H), 5.63 (d, J=5.3 Hz, 1H), 5.01-4.25 (m, 3H), 4.04-3.90 (m, 1H), 2.83 (d, J=4.5 Hz, 3H), 2.67-2.63 (m, 6H), 1.51 (s, 3H).

Isomer Example 10.11: MS: 458 [M+1]⁺. ¹H NMR (500 MHz, DMSO-d₆) δ 8.13 (s, 1H), 8.00-7.93 (m, 2H), 7.86 (d, J=8.5 Hz, 1H), 7.67 (d, J=1.9 Hz, 1H), 7.27 (dd, J=8.5, 2.0 Hz, 1H), 5.63 (d, J=5.4 Hz, 1H), 5.02-4.27 (m, 3H), 4.03-3.91 (m, 1H), 2.83 (d, J=4.6 Hz, 3H), 2.65 (s, 6H), 1.51 (s, 3H).

TABLE 14
Utilizing a method similar to that described for the preparation of Example 10.1 and
the appropriate starting materials, the following examples were prepared:

| | | | Exact |
|---|---|---|---|
| Compound | | | Mass |
| Number | Structure | Name | [M + 1]⁺ |
| Example 10.2A | 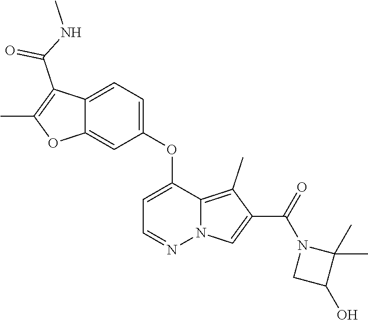 | (S or R)-6-((6-(3-hydroxy-2,2- dimethylazetidine-1-carbonyl)-5- methylpyrrolo[1,2-b]pyridazin-4- yl)oxy)-N,2-dimethylbenzofuran- 3-carboxamide | 463 |
| Example 10.2B | 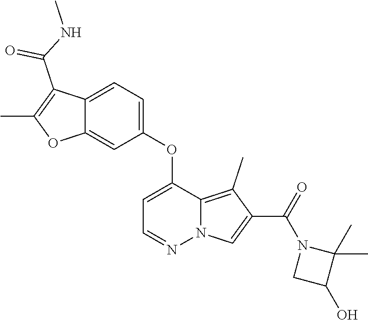 | (R or S)-6-((6-(3-hydroxy-2,2- dimethylazetidine-1-carbonyl)-5- methylpyrrolo[1,2-b]pyridazin-4- yl)oxy)-N,2-dimethylbenzofuran- 3-carboxamide | 463 |
| Example 10.3A | 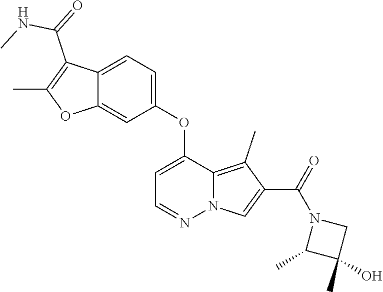 [SMILES: CNC(=O)c1c(C)oc2cc(Oc3ccnn4cc(C(=O)N5C[C@](C)(O)[C@@H]5C)c(C)c34)ccc12] | 6-({6-[(2S,3S or 2R,3R)-3- hydroxy-2,3-dimethylazetidine-1- carbonyl]-5-methylpyrrolo[1,2- b]pyridazin-4-yl}oxy)-N,2- dimethyl-1-benzofuran-3- carboxamide | 463 |
| Example 10.3B | 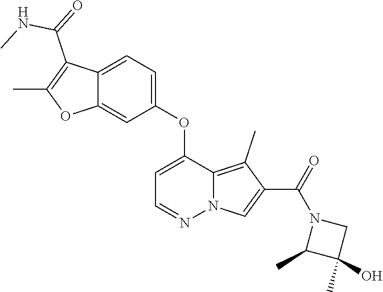 [SMILES: CNC(=O)c1c(C)oc2cc(Oc3ccnn4cc(C(=O)N5C[C@@](C)(O)[C@H]5C)c(C)c34)ccc12] | 6-({6-[(2R,3R or 2S,3S)-3- hydroxy-2,3-dimethylazetidine-1- carbonyl]-5-methylpyrrolo[1,2- b]pyridazin-4-yl}oxy)-N,2- dimethyl-1-benzofuran-3- carboxamide | 463 |
| Example 10.4A | 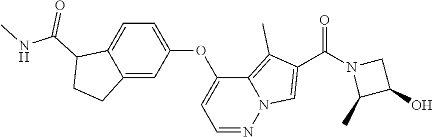 [SMILES: CNC(=O)C1CCc2cc(Oc3ccnn4cc(C(=O)N5C[C@@H](O)[C@H]5C)c(C)c34)ccc21] | (S or R)-5-({6-[(2R,3R)-3- hydroxy-2-methylazetidine-1- carbonyl]-5-methylpyrrolo[1,2- b]pyridazin-4-yl}oxy)-N-methyl- 2,3-dihydro-1H-indene-1- carboxamide | 435 |
| Example 10.5A | 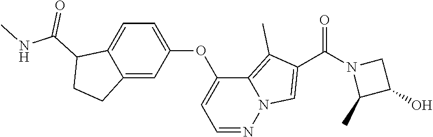 [SMILES: CNC(=O)C1CCc2cc(Oc3ccnn4cc(C(=O)N5C[C@H](O)[C@H]5C)c(C)c34)ccc21] | (S or R)-5-({6-[(2R,3S)-3- hydroxy-2-methylazetidine-1- carbonyl]-5-methylpyrrolo[1,2- b]pyridazin-4-yl}oxy)-N-methyl- 2,3-dihydro-1H-indene-1- carboxamide | 435 |

### Preparation of Example 11.1A & 11.1B: (S or R)-N,2-dimethyl-6-((5-methyl-6-(8-oxo-2,7-diazaspiro[4.4]nonane-2-carbonyl)pyrrolo[1,2-b]pyridazin-4-yl)oxy)benzofuran-3-carboxamide

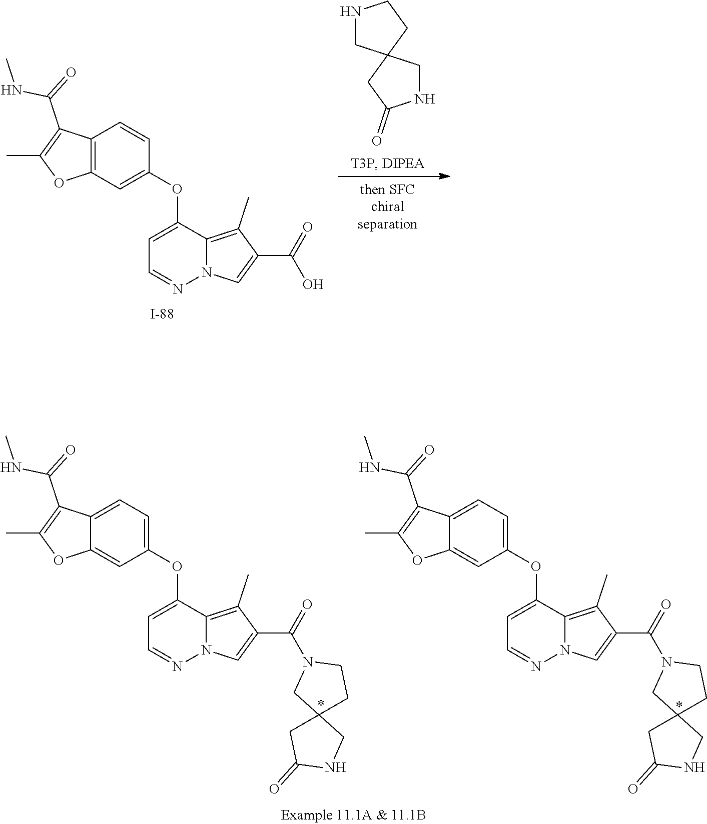

To 5-methyl-4-((2-methyl-3-(methylcarbamoyl)benzofuran-6-yl)oxy)pyrrolo[1,2-b]pyridazine-6-carboxylic acid (38 mg, 0.10 mmol) and 2,7-diazaspiro[4.4]nonan-3-one, HCl (35 mg, 0.20 mmol) in DMF (1.0 mL), DIPEA (52 μL, 0.30 mmol) and T3P® (0.13 g, 0.12 mL, 50% Wt, 0.20 mmol) were added. The reaction mixture was allowed to stir overnight at 50° C. The mixture was filtered and purified by mass triggered reverse phase HPLC (MeCN/water with 0.1% TFA modifier) to afford the title compound.

The mixture of the two stereoisomers was purified by chiral SFC (AS-H column, Mobile phase A: CO₂, B: 45% MeOH with 0.1% NH₄OH) to afford isomer Example 11.1A (faster eluting) and Example 11.1B (slower eluting).

Isomer Example 11.1A: MS: 502 [M+1]⁺. ¹H NMR (500 MHz, DMSO-d₆) δ 8.08 (d, J=34.6 Hz, 1H), 8.00-7.95 (m, 1H), 7.93 (d, J=5.4 Hz, 1H), 7.86 (d, J=8.5 Hz, 1H), 7.70-7.58 (m, 2H), 7.27 (dd, J=8.5, 2.1 Hz, 1H), 5.62 (d, J=5.4 Hz, 1H), 3.65-3.56 (m, 2H), 3.55-3.46 (m, 2H), 3.30-3.20 (m, 1H), 3.16 (q, J=9.7 Hz, 1H), 2.83 (d, J=4.6 Hz, 3H), 2.65 (s, 3H), 2.56 (s, 3H), 2.35-2.21 (m, 1H), 2.19 (s, 1H), 1.99-1.87 (m, 2H).

Isomer Example 11.1B: MS: 502 [M+1]⁺. ¹H NMR (500 MHz, DMSO-d₆) δ 8.08 (d, J=34.6 Hz, 1H), 8.01-7.95 (m, 1H), 7.93 (d, J=5.4 Hz, 1H), 7.86 (d, J=8.5 Hz, 1H), 7.71-7.57 (m, 2H), 7.27 (dd, J=8.5, 2.1 Hz, 1H), 5.62 (d, J=5.4 Hz, 1H), 3.67-3.56 (m, 2H), 3.56-3.45 (m, 2H), 3.30-3.20 (m, 1H), 3.16 (q, J=9.8 Hz, 1H), 2.83 (d, J=4.6 Hz, 3H), 2.65 (s, 3H), 2.56 (s, 3H), 2.35-2.21 (m, 1H), 2.19 (s, 1H), 2.00-1.87 (m, 2H).

TABLE 15
Utilizing a method similar to that described for the preparation of Example 11.1 and
the appropriate starting materials, the following examples were prepared:

| Compound | | | Exact Mass |
|---|---|---|---|
| Number | Structure | Name | [M + 1]⁺ |
| Example 11.2A | 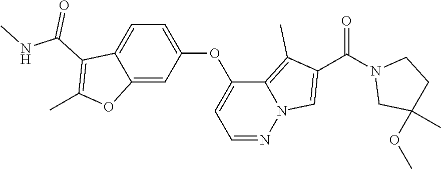 | (R or S)-6-{[6-(3-methoxy-3- methylpyrrolidine-1-carbonyl)-5- methylpyrrolo[1,2-b]pyridazin-4- yl]oxy}-N,2-dimethyl-1- benzofuran-3-carboxamide | 477 |
| Example 11.2B | 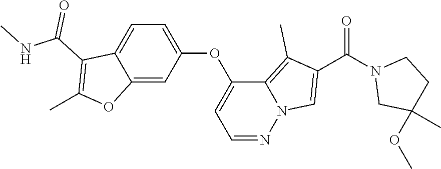 | (S or R)-6-{[6-(3-methoxy-3- methylpyrrolidine-1-carbonyl)-5- methylpyrrolo[1,2-b]pyridazin-4- yl]oxy}-N,2-dimethyl-1- benzofuran-3-carboxamide | 477 |

### Preparation of Example 12.1A & 12.1B: Cis,6-((6-(3-amino-2-methylazetidine-1-carbonyl)-5-methylpyrrolo[1,2-b]pyridazin-4-yl)oxy)-N,2-dimethylbenzofuran-3-carboxamide

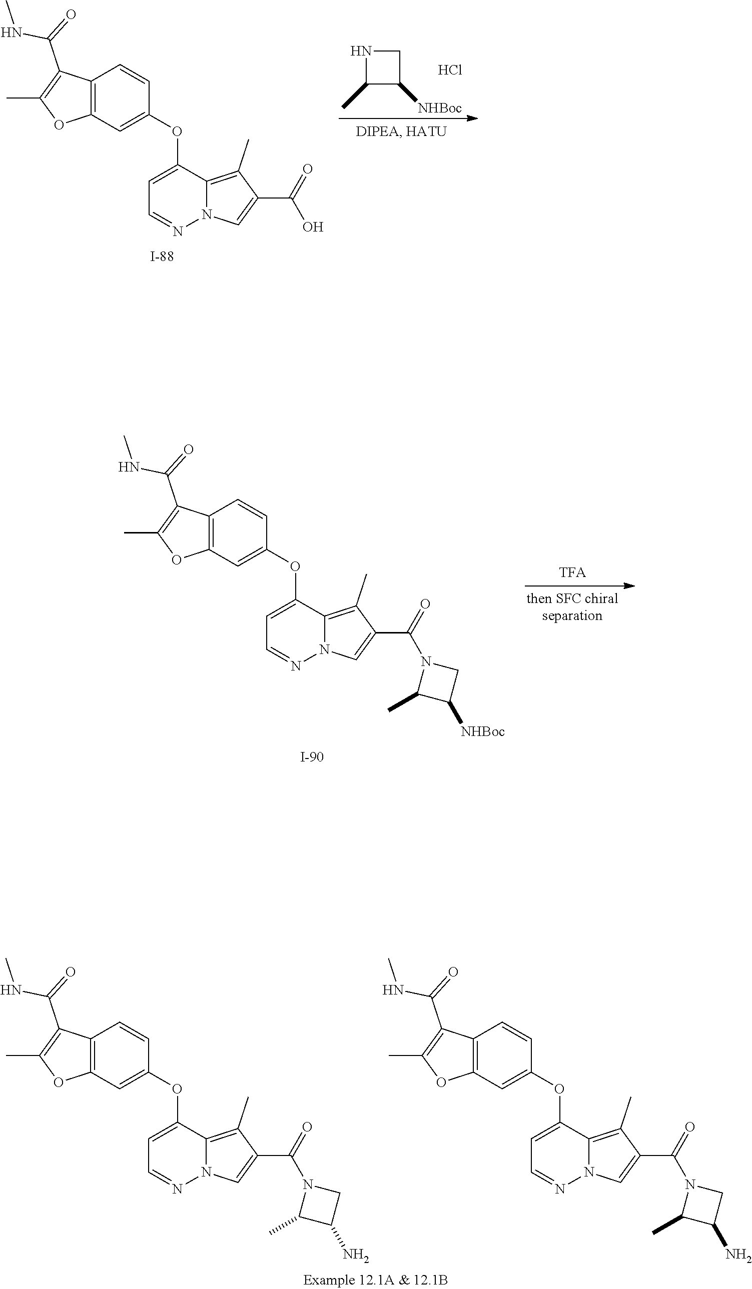

### Cis, tert-butyl (2-methyl-1-(5-methyl-4-((2-methyl-3-(methylcarbamoyl)benzofuran-6-yl)oxy)pyrrolo[1,2-b]pyridazine-6-carbonyl)azetidin-3-yl)carbamate (I-90)

A mixture of 5-methyl-4-((2-methyl-3-(methylcarbamoyl)benzofuran-6-yl)oxy)pyrrolo[1,2-b]pyridazine-6-carboxylic acid (100 mg, 0.260 mmol) and HATU (200 mg, 0.530 mmol), DIPEA (0.230 ml, 1.32 mmol), (cis)tert-butyl 2-methylazetidin-3-yl)carbamate hydrochloride (117 mg, 0.530 mmol) in DMF (2.00 mL) was stirred at 25° C. for 18 hours. The reaction mixture was quenched with water (50 mL) and extracted with EtOAc (50 mL×3). The combined organic phases were washed with LiCl (0.5 wt % aq, 50 mL), brine (50 mL), dried over anhydrous Na₂SO₄, filtered and concentrated under reduced pressure. Column chromatography (silica gel, MeOH in DCM, gradient 0 to 10%) yielded the desired product. MS: 548 [M+1]⁺. ¹H NMR (500 MHz, CD₃OD) δ 7.97-7.88 (m, 1H), 7.86 (s, 1H), 7.84 (d, J=2.7 Hz, 1H), 7.45 (s, 1H), 7.22 (d, J=6.8 Hz, 1H), 5.66 (d, J=5.4 Hz, 1H), 4.67-4.20 (m, 5H), 2.99 (s, 3H), 2.98 (d, J=2.6 Hz, 3H), 2.70 (bs, 3H), 2.68 (s, 3H), 1.48 (s, 9H).

### Cis,6-((6-(3-amino-2-methylazetidine-1-carbonyl)-5-methylpyrrolo[1,2-b]pyridazin-4-yl)oxy)-N,2-dimethylbenzofuran-3-carboxamide (Examples 12.1A & 12.1B)

To a solution of cis, tert-butyl (2-methyl-1-(5-methyl-4-((2-methyl-3-(methylcarbamoyl)benzofuran-6-yl)oxy)pyrrolo[1,2-b]pyridazine-6-carbonyl)azetidin-3-yl)carbamate (100 mg, 0.180 mmol) in DCM (2.00 mL) was added TFA (0.140 mL, 1.83 mmol). The reaction mixture was stirred at 25° C. for 18 hours.

DCM and TFA was removed under reduced pressure. The mixture was purified by mass-directed reverse phase HPLC (MeCN/water with 0.1% NH₄OH modifier), followed by chiral SFC (AS-H column, Mobile phase A: CO₂, B: 30% MeOH with 0.1% NH₄OH) to afford isomer Example 12.1A (faster eluting) and Example 12.1B (slower eluting).

Isomer Example 12.1A: MS: 448 [M+1]⁺. ¹H NMR (500 MHz, DMSO-d₆) δz 8.07-7.95 (m, 2H), 7.93 (d, J=5.4 Hz, 1H), 7.86 (d, J=8.5 Hz, 1H), 7.67 (s, 1H), 7.27 (d, J=6.9 Hz, 1H), 5.61 (d, J=5.4 Hz, 1H), 4.70-3.60 (m, 3H), 2.83 (d, J=4.6 Hz, 3H), 2.65 (s, 3H), 2.63 (bs, 3H), 1.9 (bs, 1H), 1.44-0.63 (m, 4H).

Isomer Example 12.1B: MS: 448 [M+1]⁺. ¹H NMR (500 MHz, DMSO-d₆) δz 8.07-7.95 (m, 2H), 7.93 (d, J=5.4 Hz, 1H), 7.86 (d, J=8.5 Hz, 1H), 7.67 (s, 1H), 7.27 (d, J=6.9 Hz, 1H), 5.61 (d, J=5.4 Hz, 1H), 4.70-3.60 (m, 3H), 2.83 (d, J=4.6 Hz, 3H), 2.65 (s, 3H), 2.63 (bs, 3H), 1.9 (bs, 1H), 1.44-0.63 (m, 4H).

### Preparation of Example 13.1: 1-(4-((1H-indol-5-yl)oxy)-5-methylpyrrolo[1,2-b]pyridazine-6-carbonyl)azetidine-3-carbonitrile

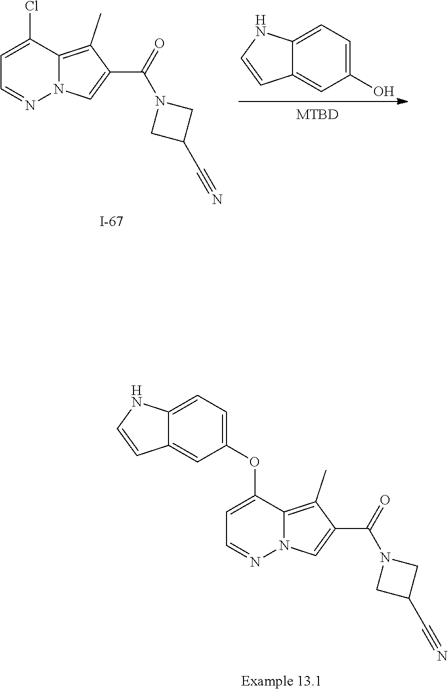

A 2 mL vial was charged with 1-(4-chloro-5-methylpyrrolo[1,2-b]pyridazine-6-carbonyl)azetidine-3-carbonitrile I-67 (15.0 mg, 54.6 mol), 5-hydroxyindole (13.5 mg, 101 μmol), MTBD (23.5 μL, 164 μmol) and DMF (1.50 mL). The vial was capped, and the reaction mixture heated to 80° C. for 16 hours. The reaction mixture was treated with Biotage® MP-Carbonate resin (200 mg) for 2 hours at room temperature. The reaction mixture was filtered and purified by mass triggered reverse phase HPLC (MeCN/water with 0.1% TFA modifier, linear gradient) to afford the desired product. MS: 372 [M+1]⁺. ¹H NMR (600 MHz, DMSO-d₆) δ 11.31 (s, 1H), 8.11 (s, 1H), 7.92 (d, J=5.4 Hz, 1H), 7.52 (d, J=8.7 Hz, 1H), 7.47 (t, J=2.8 Hz, 1H), 7.46 (d, J=2.3 Hz, 1H), 7.01-6.98 (m, 1H), 6.56-6.40 (m, 1H), 5.55 (d, J=5.4 Hz, 1H), 4.58 (bs, 2H), 4.36 (bs, 1H), 4.17 (bs, 1H), 3.86 (ddd, J=15.3, 8.7, 6.6 Hz, 1H), 2.69 (s, 3H).

TABLE 16
Utilizing a method similar to that described for the preparation of Example 13.1 and
the appropriate starting materials, the following examples were prepared:

| Example | | | Exact Mass |
|---|---|---|---|
| Number | Structure | Name | [M + 1]⁺ |
| Example 13.2 | 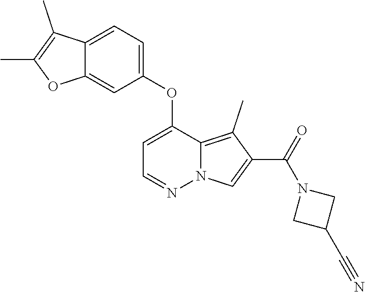 [SMILES: Cc1oc2cc(Oc3ccnn4cc(C(=O)N5CC(C#N)C5)c(C)c34)ccc2c1C] | 1-{4-[(2,3-dimethyl-1- benzofuran-6-yl)oxy]-5- methylpyrrolo[1,2- b]pyridazine-6- carbonyl}azetidine-3- carbonitrile | 401 |
| Example 13.3 | 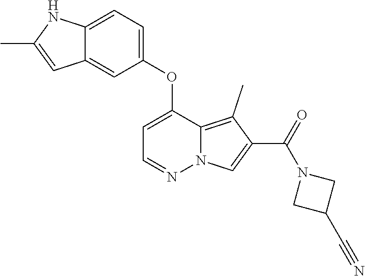 [SMILES: Cc1cc2cc(Oc3ccnn4cc(C(=O)N5CC(C#N)C5)c(C)c34)ccc2[nH]1] | 1-{5-methyl-4-[(2-methyl- 1H-indol-5- yl)oxy]pyrrolo[1,2- b]pyridazine-6- carbonyl}azetidine-3- carbonitrile | 386 |
| Example 13.4 | 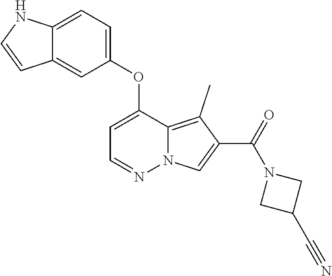 [SMILES: Cc1c(C(=O)N2CC(C#N)C2)cn2nccc(Oc3ccc4[nH]ccc4c3)c12] | 1-{4-[(1H-indol-5-yl)oxy]- 5-methylpyrrolo[1,2- b]pyridazine-6- carbonyl}azetidine-3- carbonitrile | 372 |
| Example 13.5 | 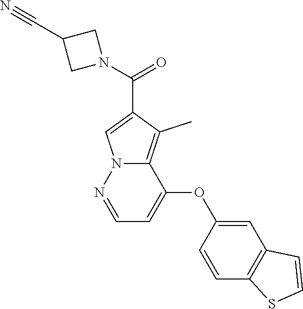 [SMILES: Cc1c(C(=O)N2CC(C#N)C2)cn2nccc(Oc3ccc4sccc4c3)c12] | 1-{4-[(1-benzothiophen-5- yl)oxy]-5- methylpyrrolo[1,2- b]pyridazine-6- carbonyl}azetidine-3- carbonitrile | 389 |
| Example 13.6 | 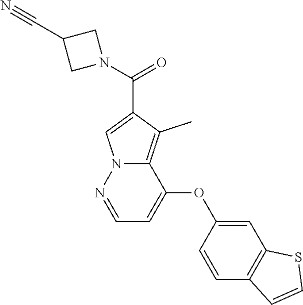 [SMILES: Cc1c(C(=O)N2CC(C#N)C2)cn2nccc(Oc3ccc4ccsc4c3)c12] | 1-{4-[(1-benzothiophen-6- yl)oxy]-5- methylpyrrolo[1,2- b]pyridazine-6- carbonyl}azetidine-3- carbonitrile | 389 |
| Example 13.7 | 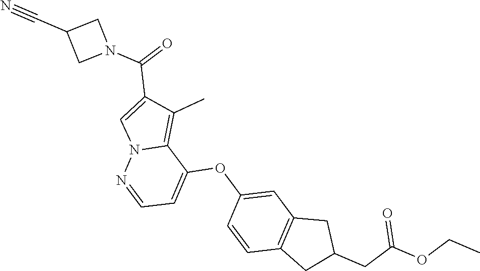 [SMILES: CCOC(=O)CC1Cc2ccc(Oc3ccnn4cc(C(=O)N5CC(C#N)C5)c(C)c34)cc2C1] | ethyl (5-{[6-(3- cyanoazetidine-1-carbonyl)- 5-methylpyrrolo[1,2- b]pyridazin-4-yl]oxy}-2,3- dihydro-1H-inden-2- yl)acetate | 459 |
| Example 13.8 | 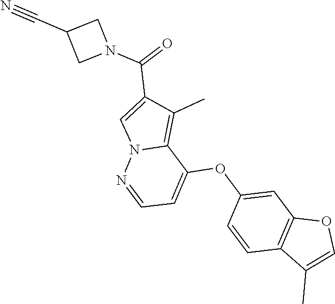 [SMILES: Cc1coc2cc(Oc3ccnn4cc(C(=O)N5CC(C#N)C5)c(C)c34)ccc12] | 1-{5-methyl-4-[(3-methyl- 1-benzofuran-6- yl)oxy]pyrrolo[1,2- b]pyridazine-6- carbonyl}azetidine-3- carbonitrile | 387 |
| Example 13.9 | 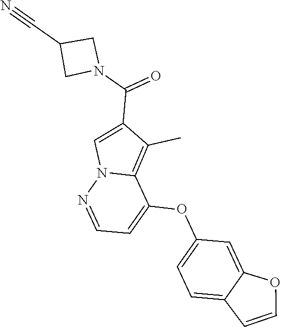 [SMILES: Cc1c(C(=O)N2CC(C#N)C2)cn2nccc(Oc3ccc4ccoc4c3)c12] | 1-{4-[(1-benzofuran-6- yl)oxy]-5- methylpyrrolo[1,2- b]pyridazine-6- carbonyl}azetidine-3- carbonitrile | 373 |
| Example 13.10 | 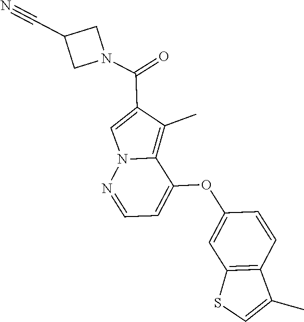 [SMILES: Cc1csc2cc(Oc3ccnn4cc(C(=O)N5CC(C#N)C5)c(C)c34)ccc12] | 1-{5-methyl-4-[(3-methyl- 1-benzothiophen-6- yl)oxy]pyrrolo[1,2- b]pyridazine-6- carbonyl}azetidine-3- carbonitrile | 403 |
| Example 13.11 | 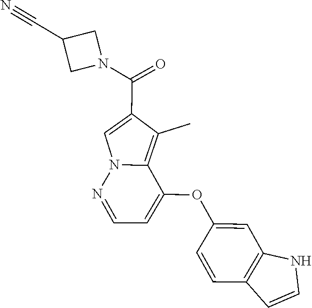 [SMILES: Cc1c(C(=O)N2CC(C#N)C2)cn2nccc(Oc3ccc4cc[nH]c4c3)c12] | 1-{4-[(1H-indol-6-yl)oxy]- 5-methylpyrrolo[1,2- b]pyridazine-6- carbonyl}azetidine-3- carbonitrile | 372 |
| Example 13.12 | 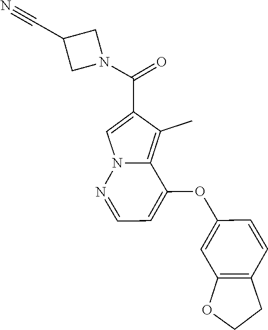 [SMILES: Cc1c(C(=O)N2CC(C#N)C2)cn2nccc(Oc3ccc4c(c3)OCC4)c12] | 1-{4-[(2,3-dihydro-1- benzofuran-6-yl)oxy]-5- methylpyrrolo[1,2- b]pyridazine-6- carbonyl}azetidine-3- carbonitrile | 375 |
| Example 13.13 | 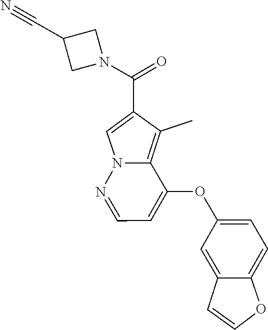 [SMILES: Cc1c(C(=O)N2CC(C#N)C2)cn2nccc(Oc3ccc4occc4c3)c12] | 1-{4-[(1-benzofuran-5- yl)oxy]-5- methylpyrrolo[1,2- b]pyridazine-6- carbonyl}azetidine-3- carbonitrile | 373 |
| Example 13.14 | 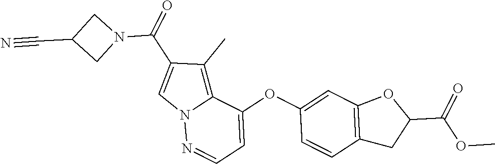 [SMILES: COC(=O)C1Cc2ccc(Oc3ccnn4cc(C(=O)N5CC(C#N)C5)c(C)c34)cc2O1] | methyl 6-{[6-(3- cyanoazetidine-1-carbonyl)- 5-methylpyrrolo[1,2- b]pyridazin-4-yl]oxy}-2,3- dihydro-1-benzofuran-2- carboxylate | 433 |
| Example 13.15 | 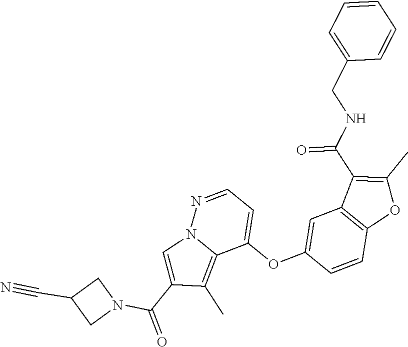 [SMILES: Cc1oc2ccc(Oc3ccnn4cc(C(=O)N5CC(C#N)C5)c(C)c34)cc2c1C(=O)NCc1ccccc1] | N-benzyl-5-{[6-(3- cyanoazetidine-1-carbonyl)- 5-methylpyrrolo[1,2- b]pyridazin-4-yl]oxy}-2- methyl-1-benzofuran-3- carboxamide | 520 |
| Example 13.16 | 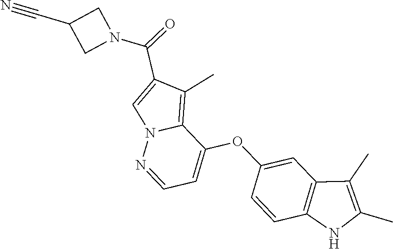 [SMILES: Cc1[nH]c2ccc(Oc3ccnn4cc(C(=O)N5CC(C#N)C5)c(C)c34)cc2c1C] | 1-{4-[(2,3-dimethyl-1H- indol-5-yl)oxy]-5- methylpyrrolo[1,2- b]pyridazine-6- carbonyl}azetidine-3- carbonitrile | 400 |
| Example 13.17 | 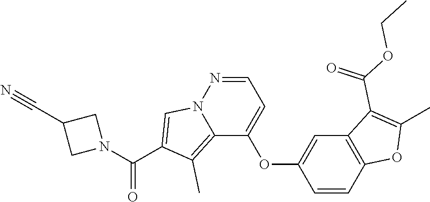 [SMILES: CCOC(=O)c1c(C)oc2ccc(Oc3ccnn4cc(C(=O)N5CC(C#N)C5)c(C)c34)cc12] | ethyl 5-{[6-(3- cyanoazetidine-1-carbonyl)- 5-methylpyrrolo[1,2- b]pyridazin-4-yl]oxy}-2- methyl-1-benzofuran-3- carboxylate | 459 |
| Example 13.18 | 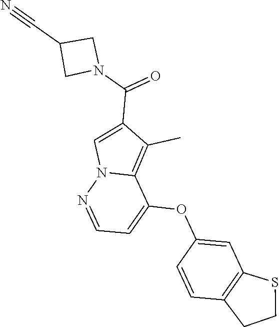 [SMILES: Cc1c(C(=O)N2CC(C#N)C2)cn2nccc(Oc3ccc4c(c3)SCC4)c12] | 1-{4-[(2,3-dihydro-1- benzothiophen-6-yl)oxy]-5- methylpyrrolo[1,2- b]pyridazine-6- carbonyl}azetidine-3- carbonitrile | 391 |

### Preparation of Example 14.1: 6-((6-(3-cyanoazetidine-1-carbonyl)-5-methylpyrrolo[1,2-b]pyridazin-4-yl)oxy)-N-cyclopropyl-2-methylbenzofuran-3-carboxamide

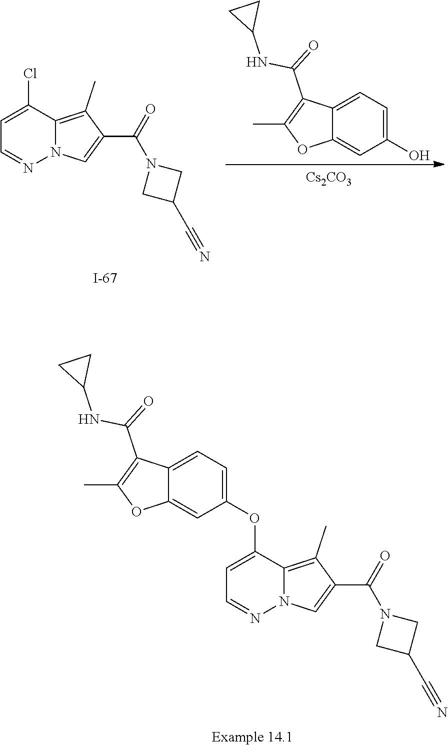

A 5 mL vial was charged with 1-(4-chloro-5-methylpyrrolo[1,2-b]pyridazine-6-carbonyl)azetidine-3-carbonitrile I-67 (25.0 mg, 91.0 mol), N-cyclopropyl-6-hydroxy-2-methylbenzofuran-3-carboxamide (23.1 mg, 100 mol), Cs₂CO₃ (32.6 mg, 100 mol) and DMF (2.00 mL). The vial was capped, and the reaction mixture heated to 80° C. for 16 hours. The reaction mixture was concentrated, diluted with EtOAc (10 mL) and washed with aqueous 0.5 wt % LiCl (10 mL×2) and water (10 mL). The organic fractions were dried (MgSO₄), filtered, and the solvent was evaporated under reduced pressure. The reaction mixture was filtered and purified by mass triggered reverse phase HPLC (MeCN/water with 0.1% TFA modifier, linear gradient) to afford the desired product. MS: 470 [M+1]⁺. ¹H NMR (600 MHz, DMSO-d₆) δ 8.18 (d, J=4.0 Hz, 1H), 8.15 (s, 1H), 7.96 (d, J=5.4 Hz, 1H), 7.76 (d, J=8.5 Hz, 1H), 7.66 (d, J=2.1 Hz, 1H), 7.25 (dd, J=8.5, 2.2 Hz, 1H), 5.62 (d, J=5.4 Hz, 1H), 4.70-4.47 (m, 2H), 4.35 (s, 1H), 4.18 (s, 1H), 3.86 (ddd, J=15.3, 8.9, 6.4 Hz, 1H), 2.88 (tq, J=7.8, 4.0 Hz, 1H), 2.66 (s, 3H), 2.61 (s, 3H), 0.75-0.71 (m, 2H), 0.64-0.60 (m, 2H).

TABLE 17
Utilizing a method similar to that described for the preparation of Example 14.1 and
the appropriate starting materials, the following examples were prepared:

| Compound | | | Exact Mass |
|---|---|---|---|
| Number | Structure | Name | [M + 1]⁺ |
| Example 14.2 | 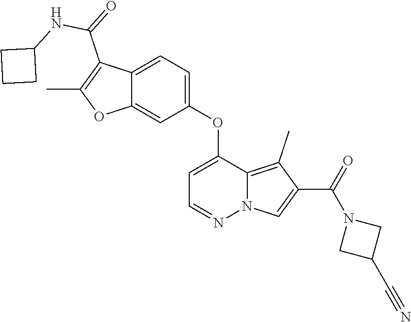 [SMILES: Cc1oc2cc(Oc3ccnn4cc(C(=O)N5CC(C#N)C5)c(C)c34)ccc2c1C(=O)NC1CCC1] | 6-{[6-(3-cyanoazetidine-1- carbonyl)-5-methylpyrrolo[1,2- b]pyridazin-4-yl]oxy}-N- cyclobutyl-2-methyl-1- benzofuran-3-carboxamide | 484 |
| Example 14.3 | 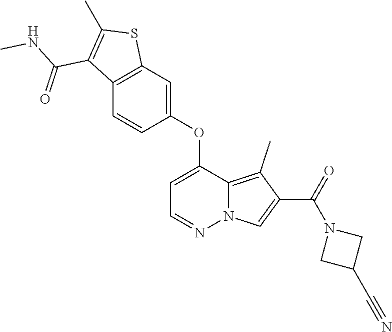 [SMILES: CNC(=O)c1c(C)sc2cc(Oc3ccnn4cc(C(=O)N5CC(C#N)C5)c(C)c34)ccc12] | 6-{[6-(3-cyanoazetidine-1- carbonyl)-5-methylpyrrolo[1,2- b]pyridazin-4-yl]oxy}-N,2- dimethyl-1-benzothiophene-3- carboxamide | 460 |
| Example 14.4 | 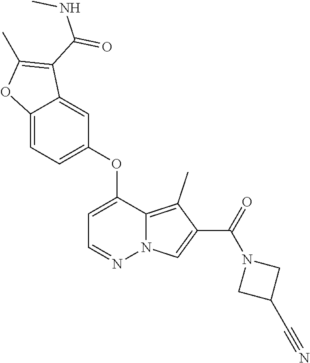 [SMILES: CNC(=O)c1c(C)oc2ccc(Oc3ccnn4cc(C(=O)N5CC(C#N)C5)c(C)c34)cc12] | 5-{[6-(3-cyanoazetidine-1- carbonyl)-5-methylpyrrolo[1,2- b]pyridazin-4-yl]oxy}-N,2- dimethyl-1-benzofuran-3- carboxamide | 444 |
| Example 14.5 | 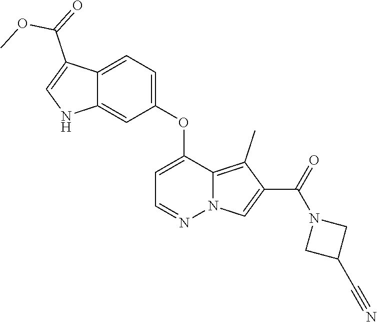 [SMILES: COC(=O)c1c[nH]c2cc(Oc3ccnn4cc(C(=O)N5CC(C#N)C5)c(C)c34)ccc12] | methyl 6-{[6-(3-cyanoazetidine- 1-carbonyl)-5-methylpyrrolo[1,2- b]pyridazin-4-yl]oxy}-1H-indole- 3-carboxylate | 430 |
| Example 14.6 | 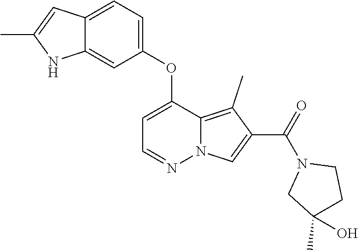 [SMILES: Cc1cc2ccc(Oc3ccnn4cc(C(=O)N5CC[C@@](C)(O)C5)c(C)c34)cc2[nH]1] | [(3R)-3-hydroxy-3- methylpyrrolidin-1-yl]{5-methyl- 4-[(2-methyl-1H-indol-6- yl)oxy]pyrrolo[1,2-b]pyridazin-6- yl}methanone | 405 |
| Example 14.7 | 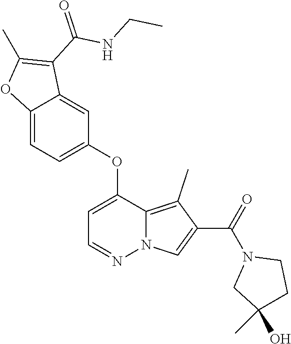 [SMILES: CCNC(=O)c1c(C)oc2ccc(Oc3ccnn4cc(C(=O)N5CC[C@@](C)(O)C5)c(C)c34)cc12] | N-ethyl-5-({6-[(3R)-3-hydroxy-3- methylpyrrolidine-1-carbonyl]-5- methylpyrrolo[1,2-b]pyridazin-4- yl}oxy)-2-methyl-1-benzofuran- 3-carboxamide | 477 |
| Example 14.8 | 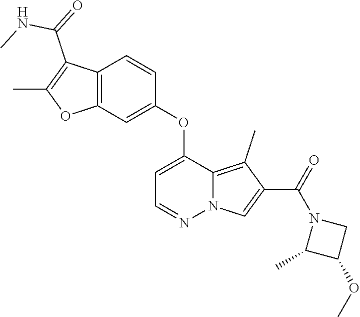 [SMILES: CNC(=O)c1c(C)oc2cc(Oc3ccnn4cc(C(=O)N5C[C@H](OC)[C@@H]5C)c(C)c34)ccc12] | 6-({6-[(2S,3S)-3-methoxy-2- methylazetidine-1-carbonyl]-5- methylpyrrolo[1,2-b]pyridazin-4- yl}oxy)-N,2-dimethyl-1- benzofuran-3-carboxamide | 463 |

### Preparation of Example 15.1: 1-{4-[(2,3-dihydro-1-benzofuran-6-yl)amino]-5-methylpyrrolo[1,2-b]pyridazine-6-carbonyl}azetidine-3-carbonitrile

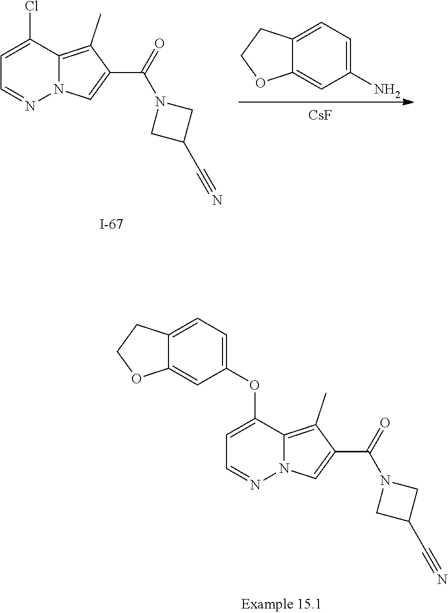

A vial was charged with 1-(4-chloro-5-methylpyrrolo[1,2-b]pyridazine-6-carbonyl)azetidine-3-carbonitrile I-67 (15.0 mg, 54.6 μmol), 2,3-dihydrobenzofuran-6-amine (29.5 mg, 218 mol), cesium fluoride (33.2 mg, 218 mol) and DMSO (1.50 mL). The reaction mixture was heated to 100° C. for 16 hours. The reaction mixture was filtered and purified by mass triggered reverse phase HPLC (MeCN/water with 0.1% TFA modifier, linear gradient) to afford the desired product. MS: 374 [M+1]⁺. ¹H NMR (500 MHz, DMSO-d₆) δ 7.95 (s, 1H), 7.94 (s, 1H), 7.79 (d, J=5.4 Hz, 1H), 7.23 (d, J=7.9 Hz, 1H), 6.79 (dd, J=7.9, 1.8 Hz, 1H), 6.74 (d, J=1.7 Hz, 1H), 5.92 (d, J=5.4 Hz, 1H), 4.56 (t, J=8.7 Hz, 2H), 4.54-4.42 (m, 2H), 4.41-4.25 (m, 1H), 4.24-4.05 (m, 1H), 3.84 (p, J=7.6 Hz, 1H), 3.16 (t, J=8.7 Hz, 2H), 2.68 (s, 3H).

TABLE 18
Utilizing a method similar to that described for the preparation of Example 15.1 and
the appropriate starting materials, the following examples were prepared:

| Example | | | Exact Mass |
|---|---|---|---|
| Number | Structure | Name | [M + 1]⁺ |
| Example 15.2 | 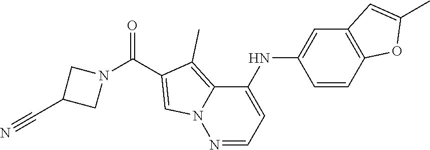 [SMILES: Cc1cc2cc(Nc3ccnn4cc(C(=O)N5CC(C#N)C5)c(C)c34)ccc2o1] | 1-{5-methyl-4-[(2-methyl-1- benzofuran-5- yl)amino]pyrrolo[1,2- b]pyridazine-6- carbonyl}azetidine-3- carbonitrile | 386 |
| Example 15.3 | 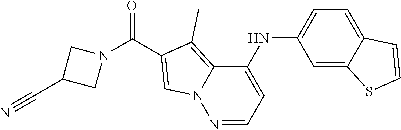 [SMILES: Cc1c(C(=O)N2CC(C#N)C2)cn2nccc(Nc3ccc4ccsc4c3)c12] | 1-{4-[(1-benzothiophen-6- yl)amino]-5-methylpyrrolo[1,2- b]pyridazine-6- carbonyl}azetidine-3- carbonitrile | 388 |
| Example 15.4 | 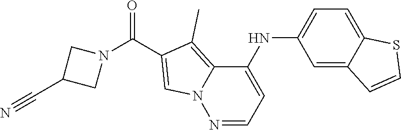 [SMILES: Cc1c(C(=O)N2CC(C#N)C2)cn2nccc(Nc3ccc4sccc4c3)c12] | 1-{4-[(1-benzothiophen-5- yl)amino]-5-methylpyrrolo[1,2- b]pyridazine-6- carbonyl}azetidine-3- carbonitrile | 388 |

### Preparation of Example 16.1: N-benzyl-6-{[6-(3-cyanoazetidine-1-carbonyl)-5-methylpyrrolo[1,2-b]pyridazin-4-yl]oxy}-2-methyl-1-benzofuran-3-carboxamide

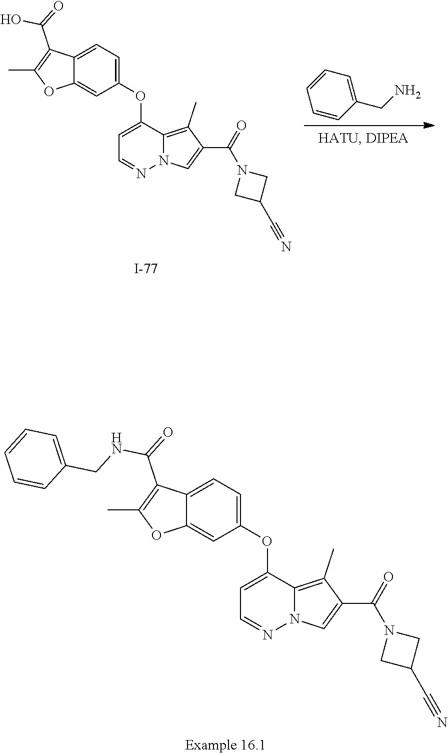

To 6-((6-(3-cyanoazetidine-1-carbonyl)-5-methylpyrrolo[1,2-b]pyridazin-4-yl)oxy)-2-methylbenzofuran-3-carboxylic acid I-77 (21.5 mg, 50.0 mol) and benzylamine (16.1 mg, 0.15 mmol) in DMF (1.67 mL), DIPEA (60.9 μL, 350 mol) and HATU (57.0 mg, 0.15 mmol) were added. The mixture was allowed to stir at 25° C. for 18 hours. The final mixture was concentrated, dissolved in DMSO and purified by mass-directed reverse phase HPLC (MeCN/water with 0.1% TFA modifier) to afford the desired product. MS: 520 [M+1]⁺.

TABLE 19
Utilizing a method similar to that described for the preparation of Example 16.1 and
the appropriate starting materials, the following examples were prepared:

| Compound | | | Exact Mass |
|---|---|---|---|
| Number | Structure | Name | [M + 1]⁺ |
| Example 16.2 | 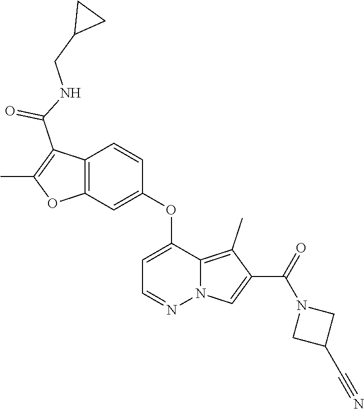 [SMILES: Cc1oc2cc(Oc3ccnn4cc(C(=O)N5CC(C#N)C5)c(C)c34)ccc2c1C(=O)NCC1CC1] | 6-{[6-(3-cyanoazetidine-1- carbonyl)-5-methylpyrrolo[1,2- b]pyridazin-4-yl]oxy}-N- (cyclopropylmethyl)-2-methyl- 1-benzofuran-3-carboxamide | 484 |
| Example 16.3 | 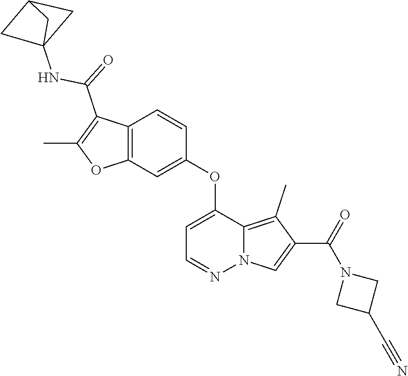 [SMILES: Cc1oc2cc(Oc3ccnn4cc(C(=O)N5CC(C#N)C5)c(C)c34)ccc2c1C(=O)NC12CC(C1)C2] | N-(bicyclo[1.1.1]pentan-1-yl)- 6-{[6-(3-cyanoazetidine-1- carbonyl)-5-methylpyrrolo[1,2- b]pyridazin-4-yl]oxy}-2- methyl-1-benzofuran-3- carboxamide | 496 |
| Example 16.4 | 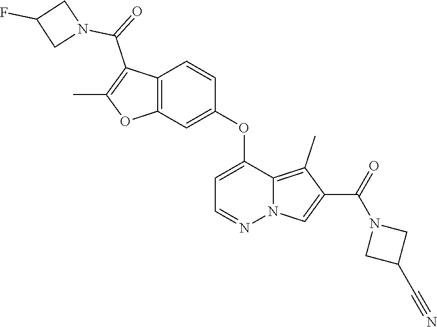 [SMILES: Cc1oc2cc(Oc3ccnn4cc(C(=O)N5CC(C#N)C5)c(C)c34)ccc2c1C(=O)N1CC(F)C1] | 1-(4-{[3-(3-fluoroazetidine-1- carbonyl)-2-methyl-1- benzofuran-6-yl]oxy}-5- methylpyrrolo[1,2- b]pyridazine-6- carbonyl)azetidine-3- carbonitrile | 488 |
| Example 16.5 | 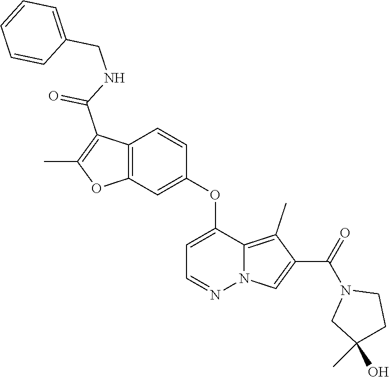 [SMILES: Cc1oc2cc(Oc3ccnn4cc(C(=O)N5CC[C@@](C)(O)C5)c(C)c34)ccc2c1C(=O)NCc1ccccc1] | N-benzyl-6-({6-[(3R)-3- hydroxy-3-methylpyrrolidine-1- carbonyl]-5-methylpyrrolo[1,2- b]pyridazin-4-yl}oxy)-2- methyl-1-benzofuran-3- carboxamide | 539 |
| Example 16.6 | 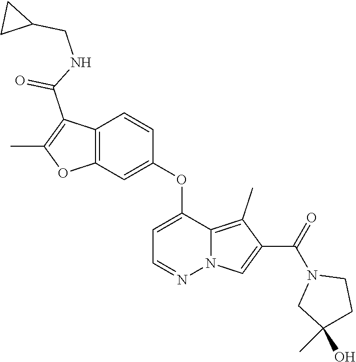 [SMILES: Cc1oc2cc(Oc3ccnn4cc(C(=O)N5CC[C@@](C)(O)C5)c(C)c34)ccc2c1C(=O)NCC1CC1] | N-(cyclopropylmethyl)-6-({6- [(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 503 |
| Example 16.7 | 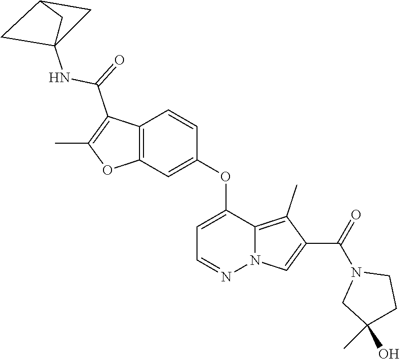 [SMILES: Cc1oc2cc(Oc3ccnn4cc(C(=O)N5CC[C@@](C)(O)C5)c(C)c34)ccc2c1C(=O)NC12CC(C1)C2] | N-(bicyclo[1.1.1]pentan-1-yl)- 6-({6-[(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 515 |
| Example 16.8 | 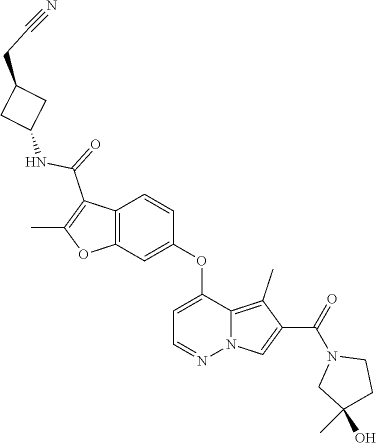 [SMILES: Cc1oc2cc(Oc3ccnn4cc(C(=O)N5CC[C@@](C)(O)C5)c(C)c34)ccc2c1C(=O)N[C@H]1C[C@H](CC#N)C1] | N-((1R,3R)-3- (cyanomethyl)cyclobutyl)-6- ((6-((R)-3-hydroxy-3- methylpyrrolidine-1-carbonyl)- 5-methylpyrrolo[1,2- b]pyridazin-4-yl)oxy)-2- methylbenzofuran-3- carboxamide | 542 |
| Example 16.9 | 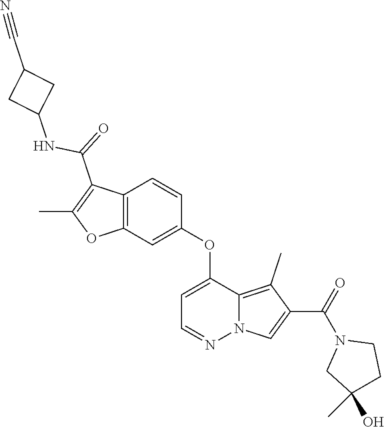 [SMILES: Cc1oc2cc(Oc3ccnn4cc(C(=O)N5CC[C@@](C)(O)C5)c(C)c34)ccc2c1C(=O)NC1CC(C#N)C1] | (cis and trans)-N-(3- cyanocyclobutyl)-6-({6-[(3R)- 3-hydroxy-3-methylpyrrolidine- 1-carbonyl]-5- methylpyrrolo[1,2-b]pyridazin- 4-yl}oxy)-2-methyl-1- benzofuran-3-carboxamide | 528 |
| Example 16.10 | 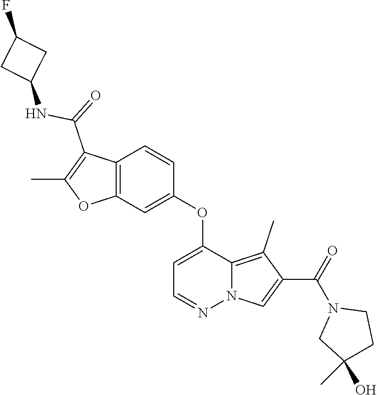 [SMILES: Cc1oc2cc(Oc3ccnn4cc(C(=O)N5CC[C@@](C)(O)C5)c(C)c34)ccc2c1C(=O)N[C@H]1C[C@@H](F)C1] | N-((1S,3S)-3-fluorocyclobutyl)- 6-((6-((R)-3-hydroxy-3- methylpyrrolidine-1-carbonyl)- 5-methylpyrrolo[1,2- b]pyridazin-4-yl)oxy)-2- methylbenzofuran-3- carboxamide | 521 |
| Example 16.11 | 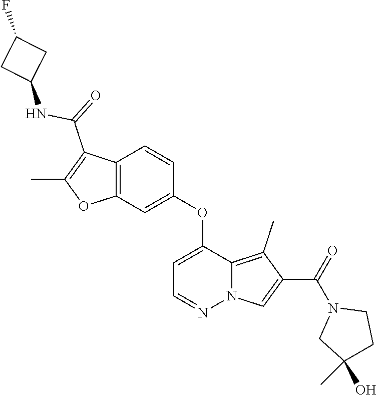 [SMILES: Cc1oc2cc(Oc3ccnn4cc(C(=O)N5CC[C@@](C)(O)C5)c(C)c34)ccc2c1C(=O)N[C@H]1C[C@H](F)C1] | N-((1R,3R)-3-fluorocyclobutyl)- 6-((6-((R)-3-hydroxy-3- methylpyrrolidine-1-carbonyl)- 5-methylpyrrolo[1,2- b]pyridazin-4-yl)oxy)-2- methylbenzofuran-3- carboxamide | 521 |
| Example 16.12 | 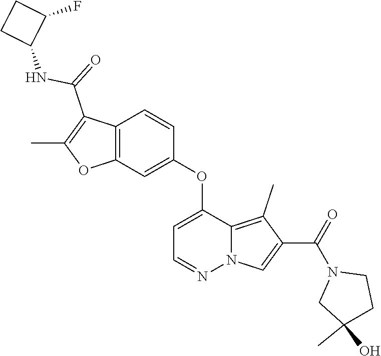 [SMILES: Cc1oc2cc(Oc3ccnn4cc(C(=O)N5CC[C@@](C)(O)C5)c(C)c34)ccc2c1C(=O)N[C@@H]1CC[C@@H]1F] | N-[(cis)-2-fluorocyclobutyl]-6- ({6-[(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 521 |
| Example 16.13 | 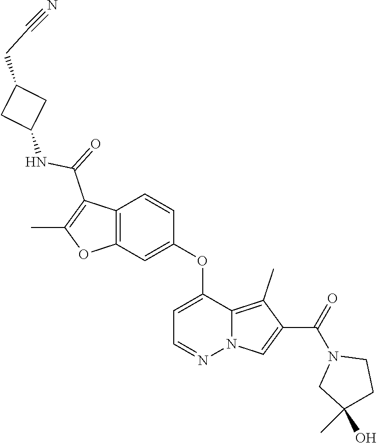 [SMILES: Cc1oc2cc(Oc3ccnn4cc(C(=O)N5CC[C@@](C)(O)C5)c(C)c34)ccc2c1C(=O)N[C@H]1C[C@@H](CC#N)C1] | N-((1S,3S)-3- (cyanomethyl)cyclobutyl)-6- ((6-((R)-3-hydroxy-3- methylpyrrolidine-1-carbonyl)- 5-methylpyrrolo[1,2- b]pyridazin-4-yl)oxy)-2- methylbenzofuran-3- carboxamide | 542 |
| Example 16.14 | 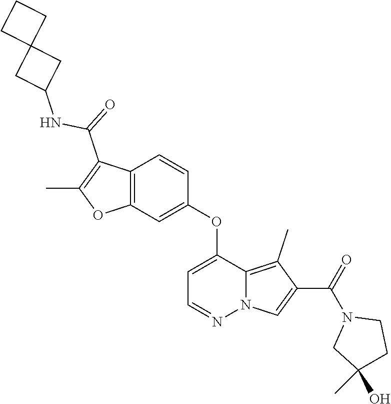 [SMILES: Cc1oc2cc(Oc3ccnn4cc(C(=O)N5CC[C@@](C)(O)C5)c(C)c34)ccc2c1C(=O)NC1CC2(CCC2)C1] | 6-({6-[(3R)-3-hydroxy-3- methylpyrrolidine-1-carbonyl]- 5-methylpyrrolo[1,2- b]pyridazin-4-yl}oxy)-2- methyl-N-(spiro[3.3]heptan-2- yl)-1-benzofuran-3- carboxamide | 543 |
| Example 16.15 | 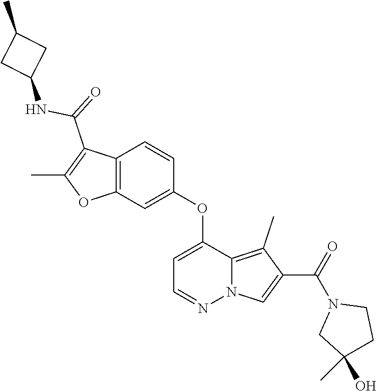 [SMILES: Cc1oc2cc(Oc3ccnn4cc(C(=O)N5CC[C@@](C)(O)C5)c(C)c34)ccc2c1C(=O)N[C@H]1C[C@@H](C)C1] | 6-((6-((R)-3-hydroxy-3- methylpyrrolidine-1-carbonyl)- 5-methylpyrrolo[1,2- b]pyridazin-4-yl)oxy)-2- methyl-N-((1S,3S)-3- methylcyclobutyl)benzofuran- 3-carboxamide | 517 |
| Example 16.16 | 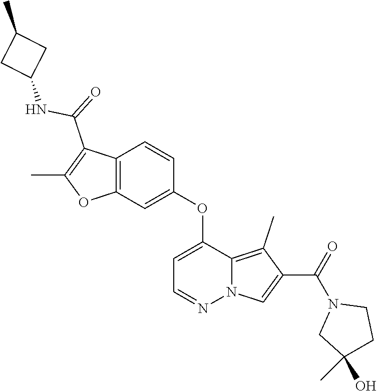 [SMILES: Cc1oc2cc(Oc3ccnn4cc(C(=O)N5CC[C@@](C)(O)C5)c(C)c34)ccc2c1C(=O)N[C@H]1C[C@H](C)C1] | 6-((6-((R)-3-hydroxy-3- methylpyrrolidine-1-carbonyl)- 5-methylpyrrolo[1,2- b]pyridazin-4-yl)oxy)-2- methyl-N-((1r,3r)-3- methylcyclobutyl)benzofuran- 3-carboxamide | 517 |
| Example 16.17 | 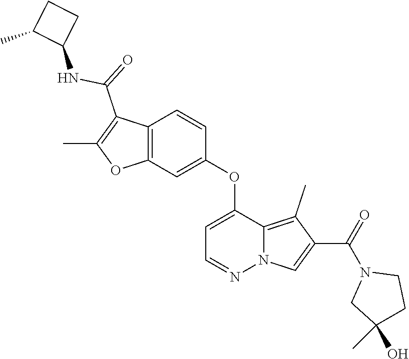 [SMILES: Cc1oc2cc(Oc3ccnn4cc(C(=O)N5CC[C@@](C)(O)C5)c(C)c34)ccc2c1C(=O)N[C@@H]1CC[C@H]1C] | 6-({6-[(3R)-3-hydroxy-3- methylpyrrolidine-1-carbonyl]- 5-methylpyrrolo[1,2- b]pyridazin-4-yl}oxy)-2- methyl-N-[(trans)-2- methylcyclobutyl]-1- benzofuran-3-carboxamide | 517 |
| Example 16.18 | 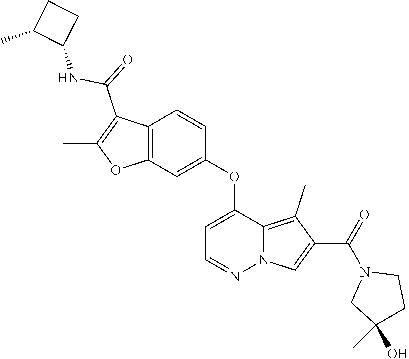 [SMILES: Cc1oc2cc(Oc3ccnn4cc(C(=O)N5CC[C@@](C)(O)C5)c(C)c34)ccc2c1C(=O)N[C@H]1CC[C@H]1C] | 6-({6-[(3R)-3-hydroxy-3- methylpyrrolidine-1-carbonyl]- 5-methylpyrrolo[1,2- b]pyridazin-4-yl}oxy)-2- methyl-N-[(cis)-2- methylcyclobutyl]-1- benzofuran-3-carboxamide | 517 |
| Example 16.19 | 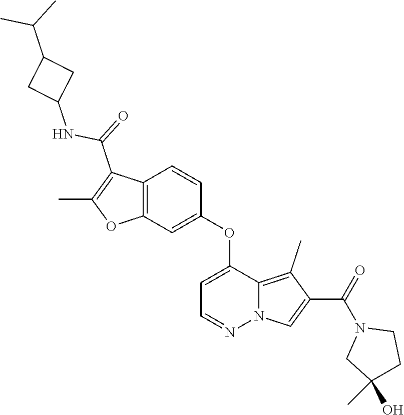 [SMILES: Cc1oc2cc(Oc3ccnn4cc(C(=O)N5CC[C@@](C)(O)C5)c(C)c34)ccc2c1C(=O)NC1CC(C(C)C)C1] | (cis and trans)-6-({6-[(3R)-3- hydroxy-3-methylpyrrolidine-1- carbonyl]-5-methylpyrrolo[1,2- b]pyridazin-4-yl}oxy)-2- methyl-N-[3-(propan-2- yl)cyclobutyl]-1-benzofuran-3- carboxamide | 545 |
| Example 16.20 | 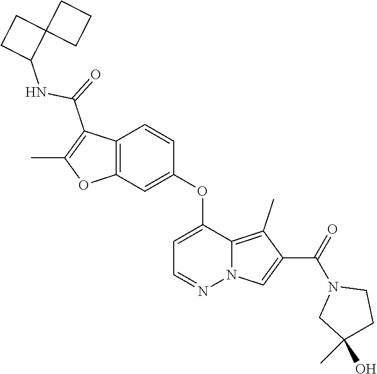 [SMILES: Cc1oc2cc(Oc3ccnn4cc(C(=O)N5CC[C@@](C)(O)C5)c(C)c34)ccc2c1C(=O)NC1CCC12CCC2] | 6-({6-[(3R)-3-hydroxy-3- methylpyrrolidine-1-carbonyl]- 5-methylpyrrolo[1,2- b]pyridazin-4-yl}oxy)-2- methyl-N-((S and R)- spiro[3.3]heptan-1-yl)-1- benzofuran-3-carboxamide | 543 |
| Example 16.21 | 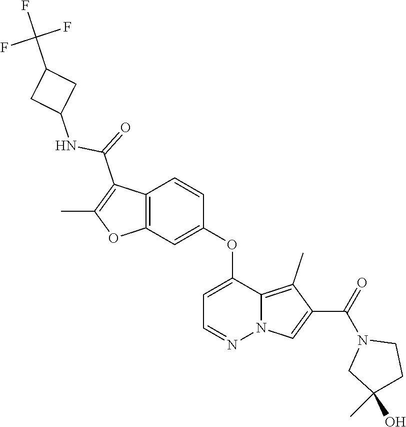 [SMILES: Cc1oc2cc(Oc3ccnn4cc(C(=O)N5CC[C@@](C)(O)C5)c(C)c34)ccc2c1C(=O)NC1CC(C(F)(F)F)C1] | (cis and trans)-6-({6-[(3R)-3- hydroxy-3-methylpyrrolidine-1- carbonyl]-5-methylpyrrolo[1,2- b]pyridazin-4-yl}oxy)-2- methyl-N-[3- (trifluoromethyl)cyclobutyl]-1- benzofuran-3-carboxamide | 571 |
| Example 16.22 | 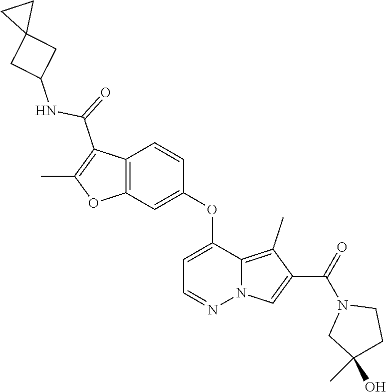 [SMILES: Cc1oc2cc(Oc3ccnn4cc(C(=O)N5CC[C@@](C)(O)C5)c(C)c34)ccc2c1C(=O)NC1CC2(CC2)C1] | 6-({6-[(3R)-3-hydroxy-3- methylpyrrolidine-1-carbonyl]- 5-methylpyrrolo[1,2- b]pyridazin-4-yl}oxy)-2- methyl-N-(spiro[2.3]hexan-5- yl)-1-benzofuran-3- carboxamide | 529 |
| Example 16.23 | 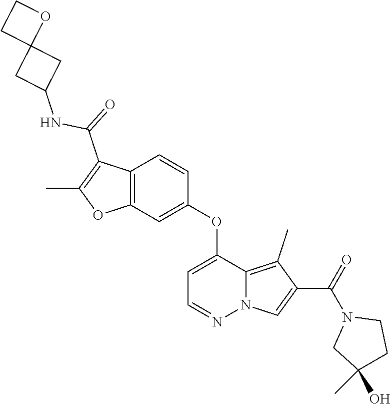 [SMILES: Cc1oc2cc(Oc3ccnn4cc(C(=O)N5CC[C@@](C)(O)C5)c(C)c34)ccc2c1C(=O)NC1CC2(CCO2)C1] | 6-({6-[(3R)-3-hydroxy-3- methylpyrrolidine-1-carbonyl]- 5-methylpyrrolo[1,2- b]pyridazin-4-yl}oxy)-2- methyl-N-(1- oxaspiro[3.3]heptan-6-yl)-1- benzofuran-3-carboxamide | 545 |
| Example 16.24 | 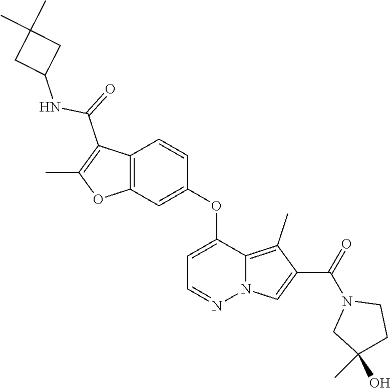 [SMILES: Cc1oc2cc(Oc3ccnn4cc(C(=O)N5CC[C@@](C)(O)C5)c(C)c34)ccc2c1C(=O)NC1CC(C)(C)C1] | N-(3,3-dimethylcyclobutyl)-6- ({6-[(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 531 |
| Example 16.25 | 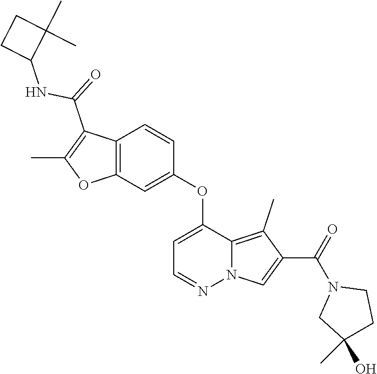 [SMILES: Cc1oc2cc(Oc3ccnn4cc(C(=O)N5CC[C@@](C)(O)C5)c(C)c34)ccc2c1C(=O)NC1CCC1(C)C] | N-({R and S}2,2- dimethylcyclobutyl)-6-({6- [(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 531 |
| Example 16.26 | 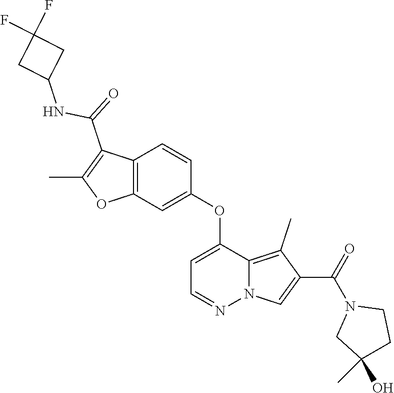 [SMILES: Cc1oc2cc(Oc3ccnn4cc(C(=O)N5CC[C@@](C)(O)C5)c(C)c34)ccc2c1C(=O)NC1CC(F)(F)C1] | N-(3,3-difluorocyclobutyl)-6- ({6-[(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 539 |
| Example 16.27 | 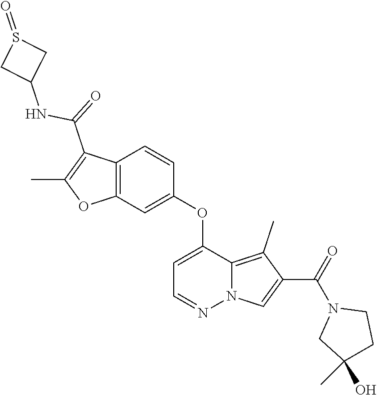 [SMILES: Cc1oc2cc(Oc3ccnn4cc(C(=O)N5CC[C@@](C)(O)C5)c(C)c34)ccc2c1C(=O)NC1CS(=O)C1] | 6-((6-((R)-3-hydroxy-3- methylpyrrolidine-1-carbonyl)- 5-methylpyrrolo[1,2- b]pyridazin-4-yl)oxy)-2- methyl-N-((1S,3S or 1R,3R)-1- oxidothietan-3-yl)benzofuran-3- carboxamide | 537 |
| Example 16.28 | 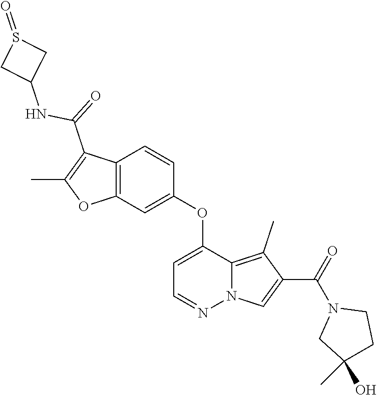 [SMILES: Cc1oc2cc(Oc3ccnn4cc(C(=O)N5CC[C@@](C)(O)C5)c(C)c34)ccc2c1C(=O)NC1CS(=O)C1] | 6-((6-((R)-3-hydroxy-3- methylpyrrolidine-1-carbonyl)- 5-methylpyrrolo[1,2- b]pyridazin-4-yl)oxy)-2- methyl-N-((1r,3r or 1s,3s)-1- oxidothietan-3-yl)benzofuran-3- carboxamide | 537 |
| Example 16.29 | 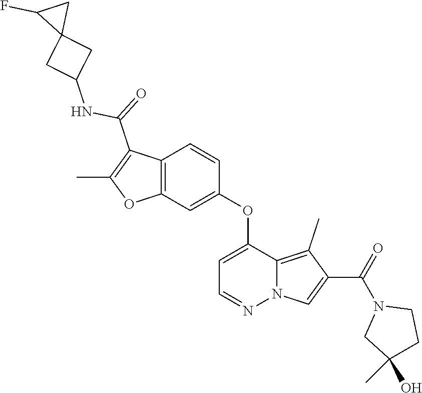 [SMILES: Cc1oc2cc(Oc3ccnn4cc(C(=O)N5CC[C@@](C)(O)C5)c(C)c34)ccc2c1C(=O)NC1CC2(C1)CC2F] | N-(1-fluorospiro[2.3]hexan-5- yl)-6-({6-[(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 547 |
| Example 16.30 | 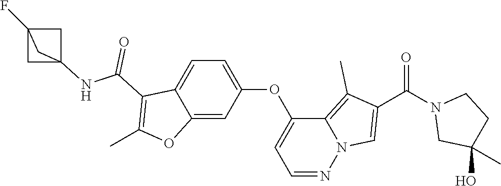 [SMILES: Cc1oc2cc(Oc3ccnn4cc(C(=O)N5CC[C@@](C)(O)C5)c(C)c34)ccc2c1C(=O)NC12CC(F)(C1)C2] | N-(3- fluorobicyclo[1.1.1]pentan-1- yl)-6-({6-[(3R)-3-hydroxy-3- methylpyrrolidine-1-carbonyl]- 5-methylpyrrolo[1,2- b]pyridazin-4-yl}oxy)-2- methyl-1-benzofuran-3- carboxamide | 533 |
| Example 16.31 | 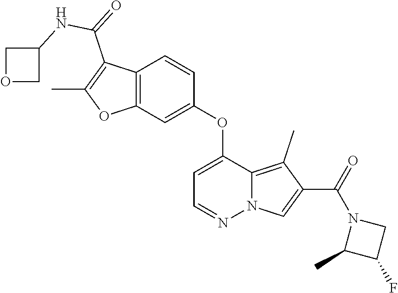 [SMILES: Cc1oc2cc(Oc3ccnn4cc(C(=O)N5C[C@H](F)[C@H]5C)c(C)c34)ccc2c1C(=O)NC1COC1] | 6-({6-[(2R,3S)-3-fluoro-2- methylazetidine-1-carbonyl]-5- methylpyrrolo[1,2-b]pyridazin- 4-yl}oxy)-2-methyl-N-(oxetan- 3-yl)-1-benzofuran-3- carboxamide | 493 |
| Example 16.32 | 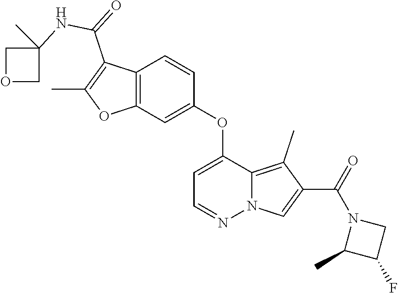 [SMILES: Cc1oc2cc(Oc3ccnn4cc(C(=O)N5C[C@H](F)[C@H]5C)c(C)c34)ccc2c1C(=O)NC1(C)COC1] | 6-({6-[(2R,3S)-3-fluoro-2- methylazetidine-1-carbonyl]-5- methylpyrrolo[1,2-b]pyridazin- 4-yl}oxy)-2-methyl-N-(3- methyloxetan-3-yl)-1- benzofuran-3-carboxamide | 507 |
| Example 16.33 | 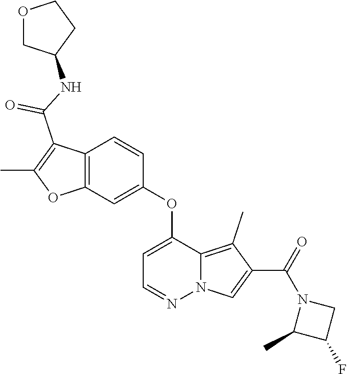 [SMILES: Cc1oc2cc(Oc3ccnn4cc(C(=O)N5C[C@H](F)[C@H]5C)c(C)c34)ccc2c1C(=O)N[C@@H]1CCOC1] | 6-({6-[(2R,3S)-3-fluoro-2- methylazetidine-1-carbonyl]-5- methylpyrrolo[1,2-b]pyridazin- 4-yl}oxy)-2-methyl-N-[(3R)- oxolan-3-yl]-1-benzofuran-3- carboxamide | 507 |
| Example 16.34 | 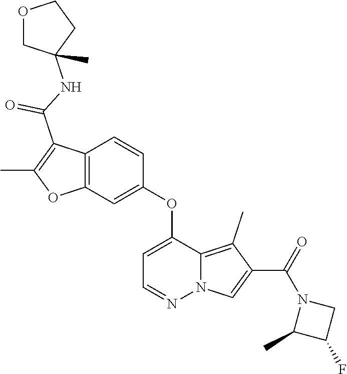 [SMILES: Cc1oc2cc(Oc3ccnn4cc(C(=O)N5C[C@H](F)[C@H]5C)c(C)c34)ccc2c1C(=O)N[C@@]1(C)CCOC1] | 6-({6-[(2R,3S)-3-fluoro-2- methylazetidine-1-carbonyl]-5- methylpyrrolo[1,2-b]pyridazin- 4-yl}oxy)-2-methyl-N-[(3S)-3- methyloxolan-3-yl]-1- benzofuran-3-carboxamide | 521 |
| Example 16.35 | 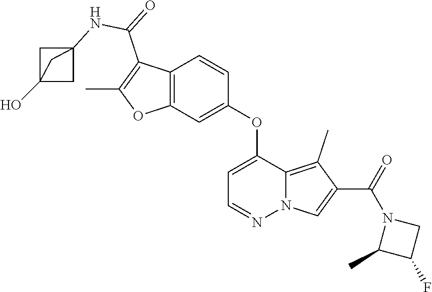 [SMILES: Cc1oc2cc(Oc3ccnn4cc(C(=O)N5C[C@H](F)[C@H]5C)c(C)c34)ccc2c1C(=O)NC12CC(O)(C1)C2] | 6-({6-[(2R,3S)-3-fluoro-2- methylazetidine-1-carbonyl]-5- methylpyrrolo[1,2-b]pyridazin- 4-yl}oxy)-N-(3- hydroxybicyclo[1.1.1]pentan-1- yl)-2-methyl-1-benzofuran-3- carboxamide | 519 |
| Example 16.36 | 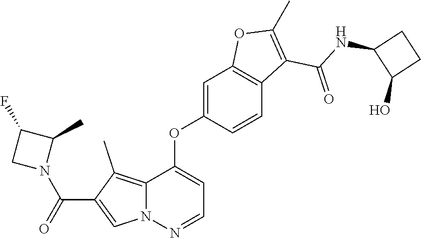 [SMILES: Cc1oc2cc(Oc3ccnn4cc(C(=O)N5C[C@H](F)[C@H]5C)c(C)c34)ccc2c1C(=O)N[C@H]1CC[C@H]1O] | 6-({6-[(2R,3S)-3-fluoro-2- methylazetidine-1-carbonyl]-5- methylpyrrolo[1,2-b]pyridazin- 4-yl}oxy)-N-[(1S,2R)-2- hydroxycyclobutyl]-2-methyl- 1-benzofuran-3-carboxamide | 507 |
| Example 16.37 | 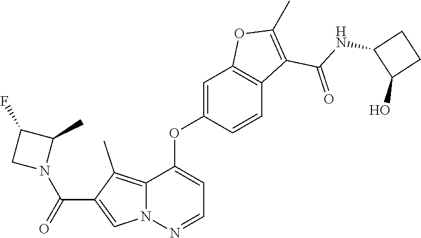 [SMILES: Cc1oc2cc(Oc3ccnn4cc(C(=O)N5C[C@H](F)[C@H]5C)c(C)c34)ccc2c1C(=O)N[C@@H]1CC[C@H]1O] | 6-({6-[(2R,3S)-3-fluoro-2- methylazetidine-1-carbonyl]-5- methylpyrrolo[1,2-b]pyridazin- 4-yl}oxy)-N-[(1R,2R)-2- hydroxycyclobutyl]-2-methyl- 1-benzofuran-3-carboxamide | 507 |
| Example 16.38 | 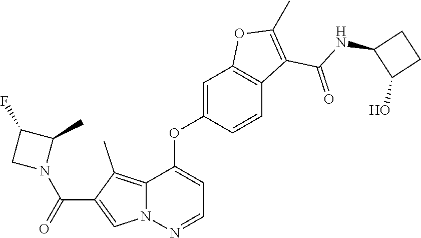 [SMILES: Cc1oc2cc(Oc3ccnn4cc(C(=O)N5C[C@H](F)[C@H]5C)c(C)c34)ccc2c1C(=O)N[C@H]1CC[C@@H]1O] | 6-({6-[(2R,3S)-3-fluoro-2- methylazetidine-1-carbonyl]-5- methylpyrrolo[1,2-b]pyridazin- 4-yl}oxy)-N-[(1S,2S)-2- hydroxycyclobutyl]-2-methyl- 1-benzofuran-3-carboxamide | 507 |
| Example 16.39 | 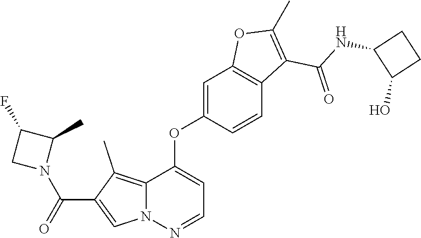 [SMILES: Cc1oc2cc(Oc3ccnn4cc(C(=O)N5C[C@H](F)[C@H]5C)c(C)c34)ccc2c1C(=O)N[C@@H]1CC[C@@H]1O] | 6-({6-[(2R,3S)-3-fluoro-2- methylazetidine-1-carbonyl]-5- methylpyrrolo[1,2-b]pyridazin- 4-yl}oxy)-N-[(1R,2S)-2- hydroxycyclobutyl]-2-methyl- 1-benzofuran-3-carboxamide | 507 |
| Example 16.40 | 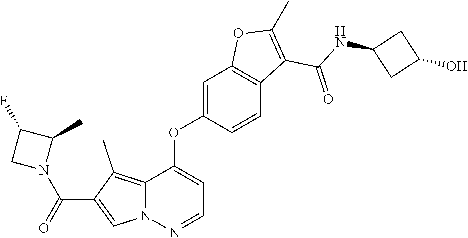 [SMILES: Cc1oc2cc(Oc3ccnn4cc(C(=O)N5C[C@H](F)[C@H]5C)c(C)c34)ccc2c1C(=O)N[C@H]1C[C@H](O)C1] | 6-({6-[(2R,3S)-3-fluoro-2- methylazetidine-1-carbonyl]-5- methylpyrrolo[1,2-b]pyridazin- 4-yl}oxy)-N-(trans-3- hydroxycyclobutyl)-2-methyl- 1-benzofuran-3-carboxamide | 507 |
| Example 16.41 | 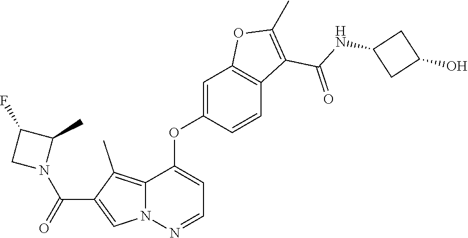 [SMILES: Cc1oc2cc(Oc3ccnn4cc(C(=O)N5C[C@H](F)[C@H]5C)c(C)c34)ccc2c1C(=O)N[C@H]1C[C@@H](O)C1] | 6-({6-[(2R,3S)-3-fluoro-2- methylazetidine-1-carbonyl]-5- methylpyrrolo[1,2-b]pyridazin- 4-yl}oxy)-N-(cis-3- hydroxycyclobutyl)-2-methyl- 1-benzofuran-3-carboxamide | 507 |
| Example 16.42 | 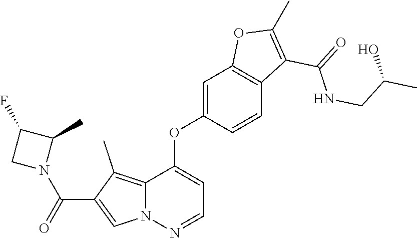 [SMILES: Cc1oc2cc(Oc3ccnn4cc(C(=O)N5C[C@H](F)[C@H]5C)c(C)c34)ccc2c1C(=O)NC[C@@H](C)O] | 6-({6-[(2R,3S)-3-fluoro-2- methylazetidine-1-carbonyl]-5- methylpyrrolo[1,2-b]pyridazin- 4-yl}oxy)-N-[(2R)-2- hydroxypropyl]-2-methyl-1- benzofuran-3-carboxamide | 495 |
| Example 16.43 | 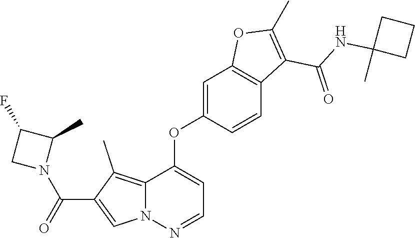 [SMILES: Cc1oc2cc(Oc3ccnn4cc(C(=O)N5C[C@H](F)[C@H]5C)c(C)c34)ccc2c1C(=O)NC1(C)CCC1] | 6-({6-[(2R,3S)-3-fluoro-2- methylazetidine-1-carbonyl]-5- methylpyrrolo[1,2-b]pyridazin- 4-yl}oxy)-2-methyl-N-(1- methylcyclobutyl)-1- benzofuran-3-carboxamide | 505 |
| Example 16.44 | 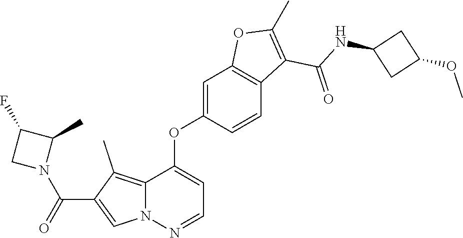 [SMILES: CO[C@H]1C[C@H](NC(=O)c2c(C)oc3cc(Oc4ccnn5cc(C(=O)N6C[C@H](F)[C@H]6C)c(C)c45)ccc23)C1] | 6-({6-[(2R,3S)-3-fluoro-2- methylazetidine-1-carbonyl]-5- methylpyrrolo[1,2-b]pyridazin- 4-yl}oxy)-N-(trans-3- methoxycyclobutyl)-2-methyl- 1-benzofuran-3-carboxamide | 521 |
| Example 16.45 | 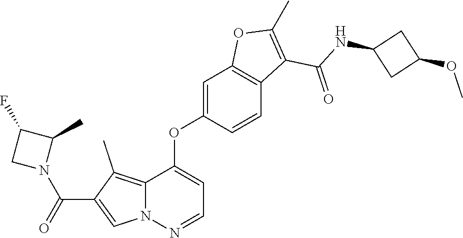 [SMILES: CO[C@H]1C[C@@H](NC(=O)c2c(C)oc3cc(Oc4ccnn5cc(C(=O)N6C[C@H](F)[C@H]6C)c(C)c45)ccc23)C1] | 6-({6-[(2R,3S)-3-fluoro-2- methylazetidine-1-carbonyl]-5- methylpyrrolo[1,2-b]pyridazin- 4-yl}oxy)-N-(cis-3- methoxycyclobutyl)-2-methyl- 1-benzofuran-3-carboxamide | 521 |

### Preparation of Example 17.1: (R)-N-(cyclopentylmethyl)-6-((6-(3-hydroxy-3-methylpyrrolidine-1-carbonyl)-5-methylpyrrolo[1,2-b]pyridazin-4-yl)oxy)-2-methylbenzofuran-3-carboxamide

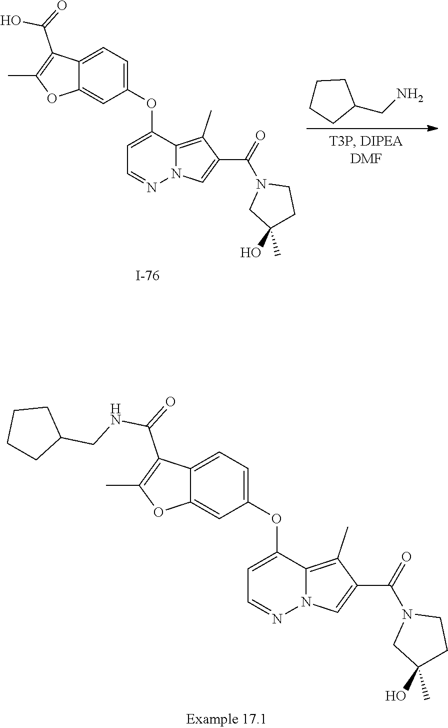

To (R)-6-((6-(3-hydroxy-3-methylpyrrolidine-1-carbonyl)-5-methylpyrrolo[1,2-b]pyridazin-4-yl)oxy)-2-methylbenzofuran-3-carboxylic acid I-76 (22.5 mg, 50.0 μmol) and cyclopentylmethanamine (14.9 mg, 0.150 mmol) in DMF (1.00 mL), DIPEA (43.5 μL, 250 μmol) and T3P® (50 wt. % in EtOAc, 89.0 μL, 0.150 mmol) were added. The mixture was allowed to stir at 50° C. for 40 hours. The final mixture was concentrated, dissolved in DMSO and purified by mass-directed reverse phase HPLC (MeCN/water with 0.1% NH₄OH modifier) to afford the desired product. MS: 531 [M+1]⁺.

TABLE 20
Utilizing a method similar to that described for the preparation of Example 17.1 and
the appropriate starting materials, the following examples were prepared:

| Com- | | | Exact |
|---|---|---|---|
| pound | | | Mass |
| Number | Structure | Name | [M + 1]⁺ |
| Example 17.2 | 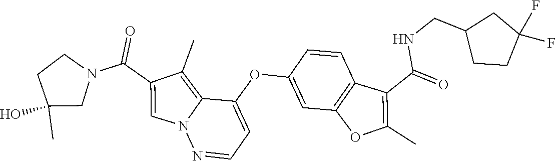 [SMILES: Cc1oc2cc(Oc3ccnn4cc(C(=O)N5CC[C@@](C)(O)C5)c(C)c34)ccc2c1C(=O)NCC1CCC(F)(F)C1] | N-[((R and S)-3,3- difluorocyclopentyl)methyl]- 6-({6-[(3R)-3-hydroxy-3- methylpyrrolidine-1- carbonyl]-5- methylpyrrolo[1,2- b]pyridazin-4-yl}oxy)-2- methyl-1-benzofuran-3- carboxamide | 567 |
| Example 17.3 | 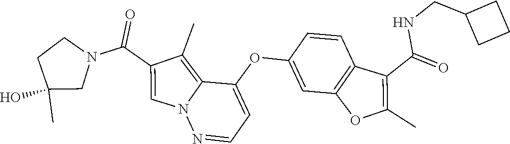 [SMILES: Cc1oc2cc(Oc3ccnn4cc(C(=O)N5CC[C@@](C)(O)C5)c(C)c34)ccc2c1C(=O)NCC1CCC1] | N-(cyclobutylmethyl)-6- ({6-[(3R)-3-hydroxy-3- methylpyrrolidine-1- carbonyl]-5- methylpyrrolo[1,2- b]pyridazin-4-yl}oxy)-2- methyl-1-benzofuran-3- carboxamide | 517 |
| Example 17.4 | 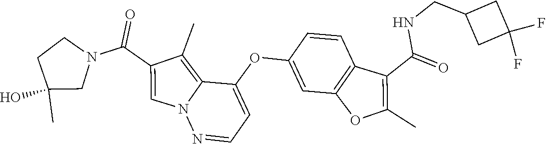 [SMILES: Cc1oc2cc(Oc3ccnn4cc(C(=O)N5CC[C@@](C)(O)C5)c(C)c34)ccc2c1C(=O)NCC1CC(F)(F)C1] | N-[(3,3- difluorocyclobutyl)methyl]- 6-({6-[(3R)-3-hydroxy-3- methylpyrrolidine-1- carbonyl]-5- methylpyrrolo[1,2- b]pyridazin-4-yl}oxy)-2- methyl-1-benzofuran-3- carboxamide | 553 |
| Example 17.5 | 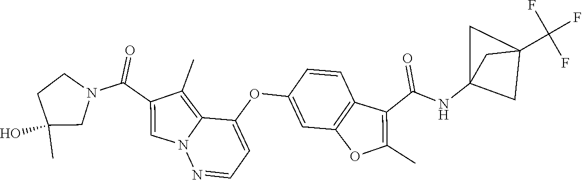 [SMILES: Cc1oc2cc(Oc3ccnn4cc(C(=O)N5CC[C@@](C)(O)C5)c(C)c34)ccc2c1C(=O)NC12CC(C(F)(F)F)(C1)C2] | 6-({6-[(3R)-3-hydroxy-3- methylpyrrolidine-1- carbonyl]-5- methylpyrrolo[1,2- b]pyridazin-4-yl}oxy)-2- methyl-N-[3- (trifluoromethyl)bicyclo [1.1.1]pentan-1-yl]-1- benzofuran-3-carboxamide | 583 |

### Preparation of Example 18.1: 6-((6-((1S,2S,4R)-2-hydroxy-2-methyl-7-azabicyclo[2.2.1]heptane-7-carbonyl)-5-methylpyrrolo[1,2-b]pyridazin-4-yl)oxy)-N,2-dimethylbenzofuran-3-carboxamide

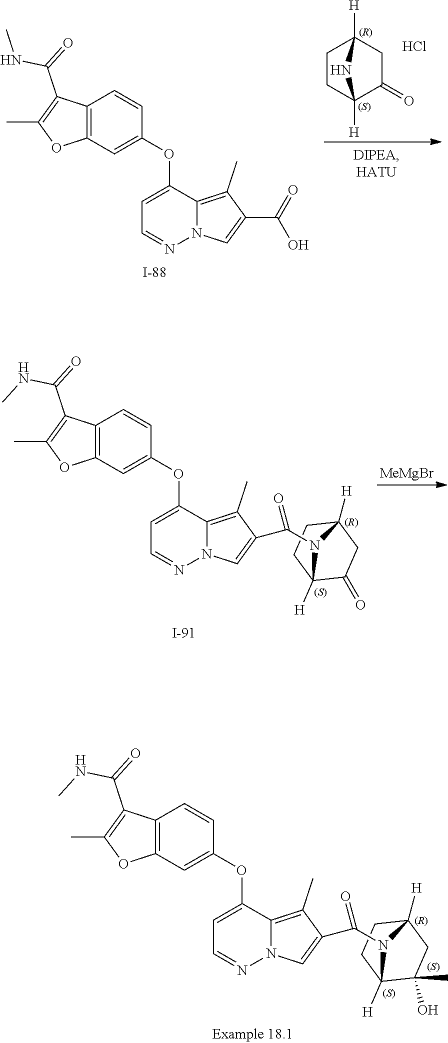

### N,2-dimethyl-6-((5-methyl-6-((1S,4R)-2-oxo-7-azabicyclo[2.2.1]heptane-7-carbonyl)pyrrolo[1,2-b]pyridazin-4-yl)oxy)benzofuran-3-carboxamide (I-91)

A mixture of 5-methyl-4-((2-methyl-3-(methylcarbamoyl)benzofuran-6-yl)oxy)pyrrolo[1,2-b]pyridazine-6-carboxylic acid (130 mg, 0.340 mmol) and HATU (261 mg, 0.680 mmol), DIPEA (0.600 ml, 3.43 mmol), (1S,4R)-7-azabicyclo[2.2.1]heptan-2-one hydrochloride (101 mg, 0.680 mmol) in DMF (2.00 mL) was stirred at 25° C. for 18 hours. The reaction mixture was quenched with water (50 mL) and extracted with EtOAc (50 mL×3). The combined organic phases were washed with LiCl (0.5 wt % aq, 50 mL), brine (50 mL), dried over anhydrous Na₂SO₄, filtered and concentrated under reduced pressure. Column chromatography (silica gel, MeOH in DCM, gradient 0 to 5%) yielded the desired product. MS: 473 [M+1]⁺. ¹H NMR (500 MHz, DMSO-d₆) δ 8.15 (s, 1H), 7.97 (t, J=4.8 Hz, 2H), 7.86 (d, J=8.5 Hz, 1H), 7.67 (d, J=2.1 Hz, 1H), 7.28 (dd, J=8.5, 2.1 Hz, 1H), 5.64 (d, J=5.4 Hz, 1H), 4.73 (bs, 1H), 4.41 (bs, 1H), 2.83 (d, J=4.6 Hz, 3H), 2.65 (s, 3H), 2.58 (s, 3H), 2.19 (d, J=17.5 Hz, 1H), 2.12-2.04 (m, 1H), 1.99-1.90 (m, 1H), 1.72 (td, J=10.5, 4.2 Hz, 1H), 1.67-1.58 (m, 1H).

### 6-((6-((1S,2S,4R)-2-hydroxy-2-methyl-7-azabicyclo[2.2.1]heptane-7-carbonyl)-5-methylpyrrolo[1,2-b]pyridazin-4-yl)oxy)-N,2-dimethylbenzofuran-3-carboxamide (Example 18.1)

N,2-dimethyl-6-((5-methyl-6-((1S,4R)-2-oxo-7-azabicyclo[2.2.1]heptane-7-carbonyl)pyrrolo[1,2-b]pyridazin-4-yl)oxy)benzofuran-3-carboxamide (35.0 mg, 70.0 μmol) was dissolved in THF (5.00 mL). The solution was cooled to 0° C. before methylmagnesium bromide (3.0 M in Et₂O, 74.0 μL, 0.220 mmol) was added. The reaction was stirred at 0° C. for 4 hours. The reaction was quenched by addition of NH₄Cl (sat aq, 50 mL). The aqueous mixture was extracted with EtOAc (50 mL). The organic mixture was washed with brine (50 mL), dried over anhydrous Na₂SO₄, filtered and concentrated under reduced pressure. The mixture was purified by mass-directed reverse phase HPLC (MeCN/water with 0.1% NH₄OH modifier) to afford the desired product. MS: 489 [M+1]⁺. ¹H NMR (500 MHz, DMSO-d₆) δ 8.04-7.92 (m, 3H), 7.86 (d, J=8.5 Hz, 1H), 7.67 (d, J=1.8 Hz, 1H), 7.28 (dd, J=8.5, 2.1 Hz, 1H), 5.63 (d, J=5.4 Hz, 1H), 4.89 (bs, 0.85H), 4.48 (bs, 0.3H), 4.07 (bs, 0.7H), 3.70 (s, 0.15H), 2.83 (d, J=4.6 Hz, 3H), 2.65 (s, 3H), 2.56 (s, 3H), 2.27-2.20 (m, 1H), 1.69 (d, J=68.7 Hz, 3H), 1.50 (s, 1H), 1.40-1.12 (m, 4H).

TABLE 21
Using methods similar to that outlined in the synthesis of Example 18.1, and the
appropriate starting materials, the following compounds were prepared:

| Compound | | | Exact Mass |
|---|---|---|---|
| Number | Structure | Name | [M + 1]⁺ |
| Example 18.2 | 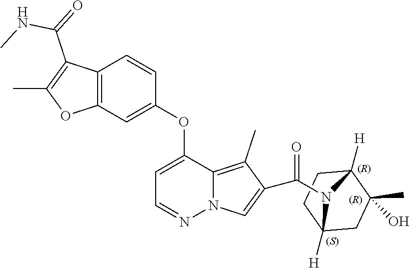 | 6-((6-((1R,2R,4S)-2- hydroxy-2-methyl-7- azabicyclo[2.2.1]heptane- 7-carbonyl)-5- methylpyrrolo[1,2- b]pyridazin-4-yl)oxy)- N,2-dimethylbenzofuran- 3-carboxamide | 489 |
| Example 18.3 | 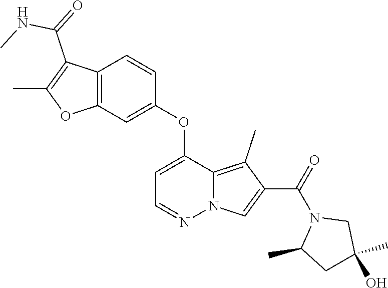 | 6-((6-((2R,4S or 2R,4R)- 4-hydroxy-2,4- dimethylpyrrolidine-1- carbonyl)-5- methylpyrrolo[1,2- b]pyridazin-4-yl)oxy)- N,2-dimethylbenzofuran- 3-carboxamide | 477 |

### Preparation of Example 19.1: 6-((6-((1S,2S,4R)-2-hydroxy-7-azabicyclo[2.2.1]heptane-7-carbonyl)-5-methylpyrrolo[1,2-b]pyridazin-4-yl)oxy)-N,2-dimethylbenzofuran-3-carboxamide

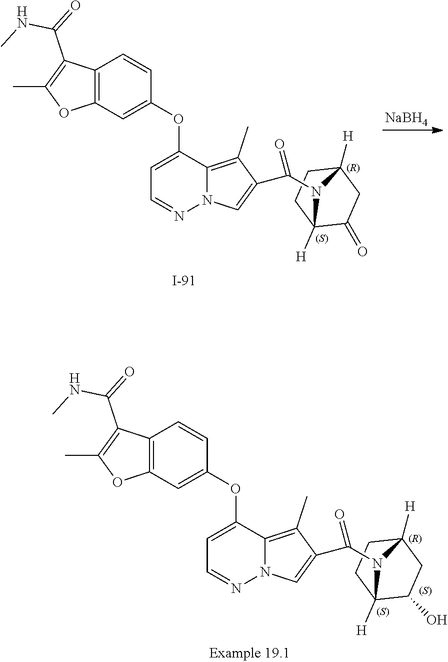

A mixture of N,2-dimethyl-6-((5-methyl-6-((1S,4R)-2-oxo-7-azabicyclo[2.2.1]heptane-7-carbonyl)pyrrolo[1,2-b]pyridazin-4-yl)oxy)benzofuran-3-carboxamide I-91 (35.0 mg, 70.0 μmol) and sodium borohydride (4.20 mg, 0.110 mmol) was dissolved in MeOH (1.00 mL). The mixture was stirred at 25° C. for 18 hours. The reaction was quenched by addition of NH₄Cl (sat aq, 20 mL). The aqueous mixture was extracted with EtOAc (20 mL). The organic mixture was washed with brine (50 mL), dried over anhydrous Na₂SO₄, filtered and concentrated under reduced pressure. The mixture was purified by mass-directed reverse phase HPLC (MeCN/water with 0.1% TFA modifier) to afford the desired product. MS: 475 [M+1]⁺. ¹H NMR (500 MHz, DMSO-d₆) δ 8.00 (s, 1H), 7.97 (q, J=4.5 Hz, 1H), 7.94 (d, J=5.4 Hz, 1H), 7.86 (d, J=8.5 Hz, 1H), 7.67 (d, J=2.1 Hz, 1H), 7.28 (dd, J=8.5, 2.1 Hz, 1H), 5.63 (d, J=5.4 Hz, 1H), 5.10 (s, 1H), 4.40 (bs, 1H), 4.19 (bs, 1H), 4.12-3.87 (m, 1H), 2.83 (d, J=4.6 Hz, 3H), 2.65 (s, 3H), 2.56 (s, 3H), 2.20-2.10 (m, 2H), 1.74 (bs, 1H), 1.65-1.47 (m, 2H).

TABLE 22
Using methods similar to that outlined in the synthesis of Example 19.1, and the
appropriate starting materials, the following compounds were prepared:

| Compound | | | Exact Mass |
|---|---|---|---|
| Number | Structure | Name | [M + 1]⁺ |
| Example 19.2 | 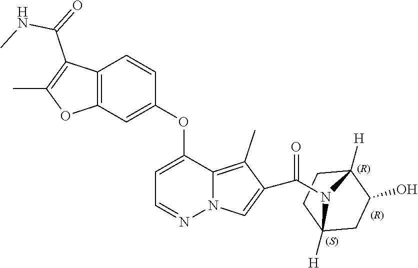 | 6-((6-((1R,2R,4S)-2- hydroxy-7- azabicyclo[2.2.1]heptane- 7-carbonyl)-5- methylpyrrolo[1,2- b]pyridazin-4-yl)oxy)- N,2-dimethylbenzofuran- 3-carboxamide | 475 |

### Preparation of Example 20.1: 5-((6-((2R,3R)-3-hydroxy-2-methylazetidine-1-carbonyl)-5-methylpyrrolo[1,2-b]pyridazin-4-yl)oxy)-N,2-dimethyl-1H-indole-1-carboxamide

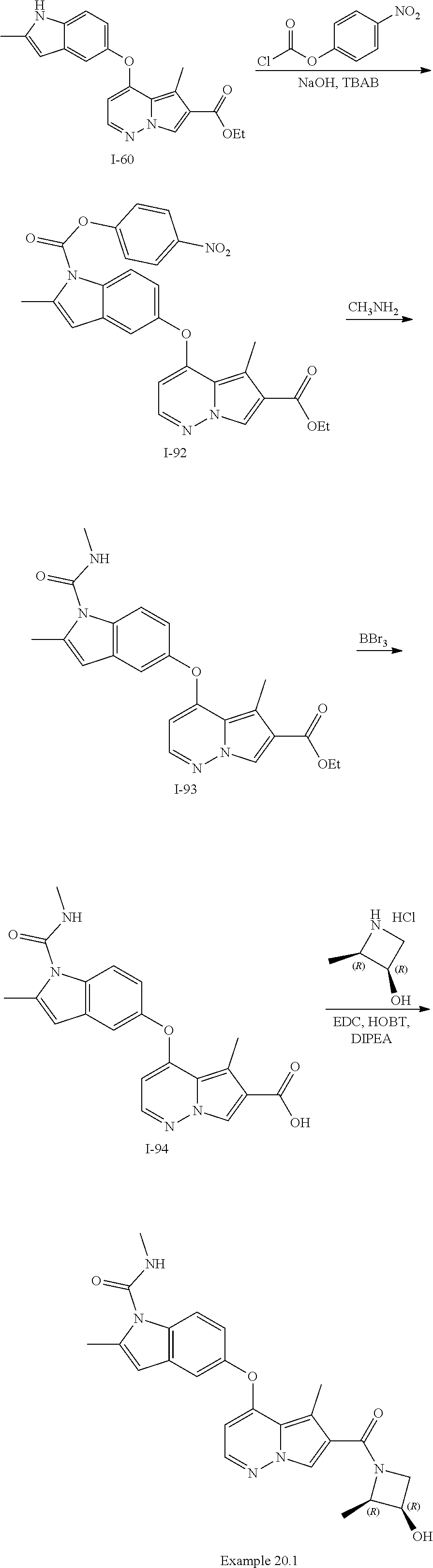

### ethyl 5-methyl-4-((2-methyl-1-((4-nitrophenoxy)carbonyl)-1H-indol-5-yl)oxy)pyrrolo[1,2-b]pyridazine-6-carboxylate (I-92)

To a solution of ethyl 5-methyl-4-((2-methyl-1H-indol-5-yl)oxy)pyrrolo[1,2-b]pyridazine-6-carboxylate (200 mg, 0.572 mmol), NaOH (68.7 mg, 1.72 mmol) and tetrabutylammonium bromide (18.4 mg, 57.0 μmol) in DCM (6.5 mL) was added 4-nitrophenyl carbonochloridate (127 mg, 0.630 mmol). The reaction mixture was stirred for 2 hours at 25° C. The mixture containing the desired product was used directly in the next step without further purification. MS: 515 [M+1]⁺.

### ethyl 5-methyl-4-((2-methyl-1-(methylcarbamoyl)-1H-indol-5-yl)oxy)pyrrolo[1,2-b]pyridazine-6-carboxylate (I-93)

To a solution of ethyl 5-methyl-4-((2-methyl-1-((4-nitrophenoxy)carbonyl)-1H-indol-5-yl)oxy)pyrrolo[1,2-b]pyridazine-6-carboxylate (295 mg, 0.401 mmol) in DCM (6.50 mL) was added methanamine (0.990 g, 31.9 mmol) (3 M in THF) at 0° C. over 1 min. After stirring for 1 hour at 25° C., the mixture was filtered, and the filtrate was concentrated under reduced pressure. Column chromatography (silica gel, EtOAc in petroleum ether, gradient 0 to 37%) yielded the desired product. MS: 407 [M+1]⁺.

### 5-methyl-4-((2-methyl-1-(methylcarbamoyl)-1H-indol-5-yl)oxy)pyrrolo[1,2-b]pyridazine-6-carboxylic acid (I-94)

To a solution of ethyl 5-methyl-4-((2-methyl-1-(methylcarbamoyl)-1H-indol-5-yl)oxy)pyrrolo[1,2-b]pyridazine-6-carboxylate (150 mg, 0.369 mmol) in DCM (7.20 mL) was added BBr₃ (2.40 mL, 25.4 mmol) at 0° C. over 3 min to give a brown mixture. The reaction mixture was stirred for 16 hours at 25° C. The mixture was quenched with ice water (30 mL) and extracted with DCM/MeOH=10:1 (20 mL×3). The combined organic phases were dried over anhydrous Na₂SO₄, filtered and concentrated under reduced pressure to afford the desired product. The material was used directly in the next step without further purification. MS: 379 [M+1]⁺.

### 5-((6-((2R,3R)-3-hydroxy-2-methylazetidine-1-carbonyl)-5-methylpyrrolo[1,2-b]pyridazin-4-yl)oxy)-N,2-dimethyl-1H-indole-1-carboxamide (Example 20.1)

A mixture of 5-methyl-4-((2-methyl-1-(methylcarbamoyl)-1H-indol-5-yl)oxy)pyrrolo[1,2-b]pyridazine-6-carboxylic acid (80.0 mg, 85.0 μmol), (2R,3R)-2-methylazetidin-3-ol hydrochloride (13.6 mg, 0.110 mmol), HOBT (34.3 mg, 0.254 mmol), DIPEA (0.118 mL, 0.677 mmol) and EDC (48.6 mg, 0.254 mmol) in DMF (0.500 mL) was stirred at 25° C. for 3 hours. The mixture was purified by mass-directed reverse phase HPLC (MeCN/water with 0.1% TFA modifier) to afford the desired product. MS: 448 [M+1]⁺. ¹H NMR (400 MHz, CD₃OD) δ 7.86 (s, 1H), 7.79 (d, J=5.2 Hz, 1H), 7.71 (d, J=8.8 Hz, 1H), 7.31 (d, J=2.4 Hz, 1H), 7.03 (dd, J=8.8, 2.4 Hz, 1H), 6.38 (s, 1H), 5.58 (d, J=5.2 Hz, 1H), 4.75-4.57 (m, 2H), 4.37 (d, J=7.2 Hz, 1H), 4.21-3.89 (m, 1H), 3.02 (s, 3H), 2.69 (s, 3H), 2.55 (s, 3H), 1.51-1.13 (m, 3H).

TABLE 3
Using methods similar to that outlined in the synthesis of Example 20.1, and the
appropriate starting materials, the following compounds were prepared:

| Compound | | | Exact Mass |
|---|---|---|---|
| Number | Structure | Name | [M + 1]⁺ |
| Example 20.2 | 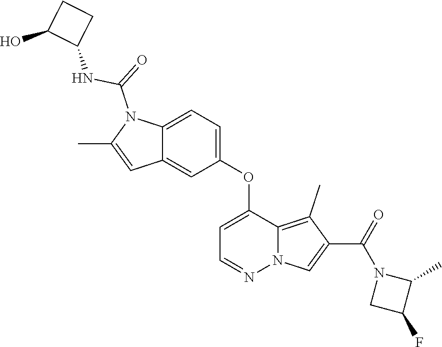 [SMILES: Cc1c(C(=O)N2C[C@H](F)[C@H]2C)cn2nccc(Oc3ccc4c(c3)cc(C)n4C(=O)N[C@H]3CC[C@@H]3O)c12] | 5-((6-((2R,3S)-3-fluoro- 2-methylazetidine-1- carbonyl)-5- methylpyrrolo[1,2- b]pyridazin-4-yl)oxy)-N- ((1S,2S)-2- hydroxycyclobutyl)-2- methyl-1H-indole-1- carboxamide | 506 |

### Preparation of Example 21.1: N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-carboxamide

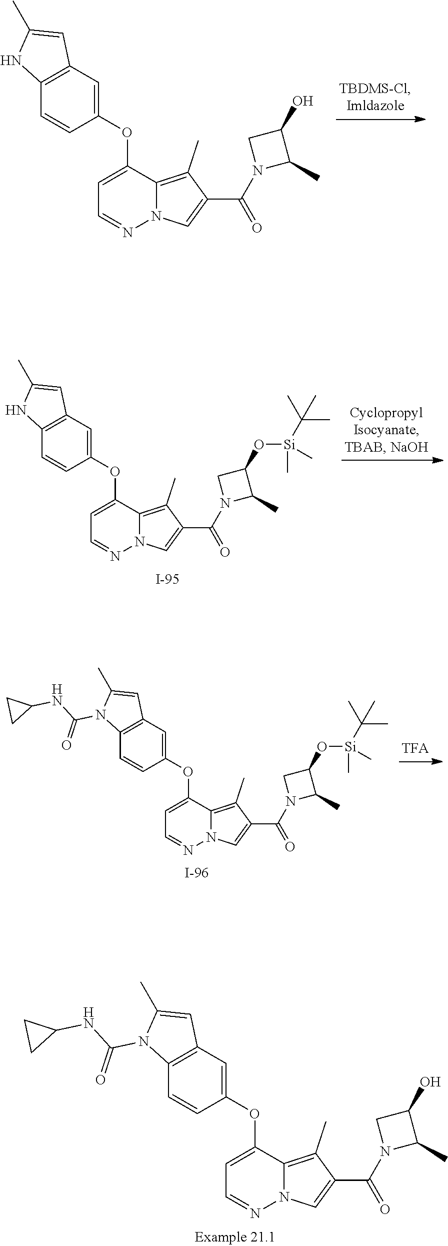

### ((2R,3R)-3-((tert-butyldimethylsilyl)oxy)-2-methylazetidin-1-yl)(5-methyl-4-((2-methyl-1H-indol-5-yl)oxy)pyrrolo[1,2-b]pyridazin-6-yl)methanone (I-9)

Starting ((2R,3R)-3-hydroxy-2-methylazetidin-1-yl)(5-methyl-4-((2-methyl-1H-indol-5-yl)oxy)pyrrolo[1,2-b]pyridazin-6-yl)methanone was readily accessed from intermediate via hydrolysis and amide coupling under standard conditions. To a solution of ((2R,3R)-3-hydroxy-2-methylazetidin-1-yl)(5-methyl-4-((2-methyl-1H-indol-5-yl)oxy)pyrrolo[1,2-b]pyridazin-6-yl)methanone (90 mg, 0.231 mmol) and tert-butylchlorodimethylsilane (45 mg, 0.300 mmol) in DCM (2 ml) was added 1H-imidazole (47 mg, 0.692 mmol) at 25° C. The reaction was stirred at 25° C. for 16 h. The reaction mixture was quenched with brine (10 mL) and extracted with DCM (5 mL×3). The organic phases were combined and concentrated under reduced pressure below 30° C. to give a residue. The residue was purified by flash chromatography over silica gel (Pet. ether/EtOAc, 1:1) to give the title compound. MS: 505 [M+1]⁺. ¹H NMR (400 MHz, CDCl₃) δ=8.07 (s, 1H), 7.75 (s, 1H), 7.69 (d, J=5.2 Hz, 1H), 7.34-7.30 (m, 2H), 6.95-6.90 (m, 1H), 6.25 (s, 1H), 5.52 (d, J=5.2 Hz, 1H), 4.85-4.63 (m, 2H), 4.34 (s, 1H), 4.02 (s, 1H), 2.97 (s, 1H), 2.89 (s, 1H), 2.78 (s, 3H), 2.49 (s, 3H), 1.48 (s, 2H), 1.30-1.23 (m, 1H), 0.92 (s, 9H), 0.09-0.06 (m, 6H).

### 5-((6-((2R,3R)-3-((tert-butyldimethylsilyl)oxy)-2-methylazetidine-1-carbonyl)-5-methylpyrrolo[1,2-b]pyridazin-4-yl)oxy)-N-cyclopropyl-2-methyl-1H-indole-1-carboxamide (I-10)

To a solution of ((2R,3R)-3-((tert-butyldimethylsilyl)oxy)-2-methylazetidin-1-yl)(5-methyl-4-((2-methyl-1H-indol-5-yl)oxy)pyrrolo[1,2-b]pyridazin-6-yl)methanone (60 mg, 0.119 mmol), tetrabutylammonium bromide (3.83 mg, 0.012 mmol) and sodium hydroxide (4.75 mg, 0.119 mmol) in DCM (2 ml) was added isocyanatocyclopropane (14.82 mg, 0.178 mmol) at 25° C. The reaction was stirred at 25° C. for 0.5 h. LCMS showed desired compound was detected. The reaction mixture was filtered, and the cake was washed with DCM (4 mL). The organic phases was combined and purified by flash chromatography over silica gel (PE:EA, 1:2) to give the title compound. MS: 588 [M+1]⁺. ¹H NMR (400 MHz, CD₃OD) δ=7.92-7.84 (m, 1H), 7.81-7.74 (m, 1H), 7.67-7.59 (m, 1H), 7.33-7.25 (m, 1H), 7.05-6.96 (m, 1H), 6.41-6.35 (m, 1H), 5.62-5.52 (m, 1H), 4.78-4.68 (m, 2H), 4.45-4.31 (m, 1H), 4.25-3.90 (m, 1H), 2.95-2.86 (m, 1H), 2.73-2.66 (m, 3H), 2.53 (s, 3H), 1.46 (s, 2H), 1.34-1.29 (m, 1H), 0.95 (s, 9H), 0.89-0.85 (m, 2H), 0.74 (d, J=2.4 Hz, 2H), 0.12-0.08 (m, 6H)

### N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-carboxamide (Example 21.1)

To a solution of 5-((6-((2R,3R)-3-((tert-butyldimethylsilyl)oxy)-2-methylazetidine-1-carbonyl)-5-methylpyrrolo[1,2-b]pyridazin-4-yl)oxy)-N-cyclopropyl-2-methyl-1H-indole-1-carboxamide (40 mg, 0.068 mmol) in DCM (0.5 ml) was added TFA (1 mL) and Water (0.05 ml) at 25° C. The reaction was stirred at 25° C. for 2 h. The reaction mixture was concentrated under reduced pressure below 30° C. to give a residue. The residue was purified by prep-HPLC (water/MeCN with 0.1% TFA) to give the title compound. MS: 474 [M+1]⁺. ¹H NMR (400 MHz, CD₃OD) δ=7.87 (s, 1H), 7.79 (d, J=5.2 Hz, 1H), 7.65 (d, J=8.8 Hz, 1H), 7.30 (d, J=2.0 Hz, 1H), 7.03 (dd, J=2.0, 8.8 Hz, 1H), 6.38 (s, 1H), 5.57 (d, J=5.2 Hz, 1H), 4.78-4.57 (m, 2H), 4.44-4.30 (m, 1H), 4.24-3.89 (m, 1H), 2.94-2.86 (m, 1H), 2.69 (s, 3H), 2.53 (s, 3H), 1.50-1.14 (m, 3H) (atropisomer), 0.90-0.85 (m, 2H), 0.76-0.71 (m, 2H),

### Preparation of Example 22.1: ethyl 4-((1-(N-cyclopropylsulfamoyl)-2-methyl-1H-indol-5-yl)oxy)-5-methylpyrrolo[1,2-b]pyridazine-6-carboxylate

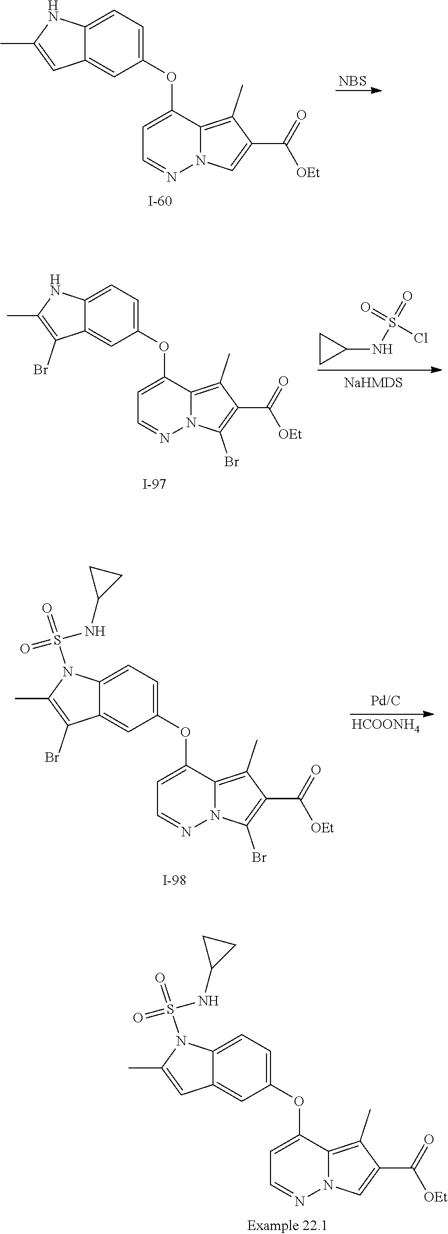

### ethyl 7-bromo-4-((3-bromo-2-methyl-1H-indol-5-yl)oxy)-5-methylpyrrolo[1,2-b]pyridazine-6-carboxylate (I-97)

To a solution of ethyl 5-methyl-4-((2-methyl-1H-indol-5-yl)oxy)pyrrolo[1,2-b]pyridazine-6-carboxylate I-60 (4.20 g, 12.0 mmol) in DMF (35.0 mL) was added NBS (4.28 g, 24.0 mmol) at 25° C. over 1 min, then the mixture was stirred for 1 hour at 25° C. The reaction mixture was quenched with water (70 mL) and extracted with EtOAc (30 mL×3). The combined organic phases were washed with brine (30 mL), dried over anhydrous Na₂SO₄, filtered and concentrated under reduced pressure. Column chromatography (silica gel, EtOAc in petroleum ether, gradient 20 to 100%) yielded the desired product. MS: 508 [M+1]⁺.

### ethyl 4-((1-(N-cyclopropylsulfamoyl)-2-methyl-1H-indol-5-yl)oxy)-5-methylpyrrolo[1,2-b]pyridazine-6-carboxylate (I-98)

To a solution of ethyl 7-bromo-4-((3-bromo-2-methyl-1H-indol-5-yl)oxy)-5-methylpyrrolo[1,2-b]pyridazine-6-carboxylate (300 mg, 0.592 mmol) in THF (3.00 ml) was added NaHMDS (1.78 ml, 1.78 mmol) (1 M in THF) at 0° C. over 1 min. After stirring for 0.5 hours at 0° C., cyclopropylsulfamoyl chloride (184 mg, 1.18 mmol) was added to the mixture at 0° C. The resulting mixture was stirred for another 1 hour. The reaction mixture was quenched with water (5 mL) and extracted with EtOAc (3 mL×3). The combined organic phases were washed with brine (5 mL), dried over anhydrous Na₂SO₄, filtered and concentrated under reduced pressure. Column chromatography (silica gel, EtOAc in petroleum ether, gradient 0 to 10%) yielded the desired product. MS: 627 [M+1]⁺.

### ethyl 4-((1-(N-cyclopropylsulfamoyl)-2-methyl-1H-indol-5-yl)oxy)-5-methylpyrrolo[1,2-b]pyridazine-6-carboxylate (Example 22.1)

A mixture of ethyl 7-bromo-4-((3-bromo-1-(N-cyclopropylsulfamoyl)-2-methyl-1H-indol-5-yl)oxy)-5-methylpyrrolo[1,2-b]pyridazine-6-carboxylate (10.0 mg, 16.0 μmol), Pd—C (1.70 mg, 1.60 μmol) (10%) and ammonium formate (5.03 mg, 80.0 μmol) in DMF (280 μL) and water (20.0 μL) was stirred at 25° C. for 1 hour. The mixture was purified by mass-directed reverse phase HPLC (MeCN/water with 0.1% TFA modifier) to afford the desired product. MS: 469 [M+1]⁺. ¹H NMR (400 MHz, CD₃OD) δ 8.11 (s, 1H), 8.06 (d, J=8.9 Hz, 1H), 7.83 (d, J=5.5 Hz, 1H), 7.35 (d, J=2.4 Hz, 1H), 7.12-7.06 (m, 1H), 6.48 (s, 1H), 5.58 (d, J=5.4 Hz, 1H), 4.38-4.32 (m, 2H), 2.81 (s, 3H), 2.63 (s, 3H), 2.20-2.13 (m, 1H), 1.40 (t, J=7.2 Hz, 3H), 0.47-0.42 (m, 2H), 0.28-0.24 (m, 2H).

### Preparation of Example 23.1: N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-sulfonamide

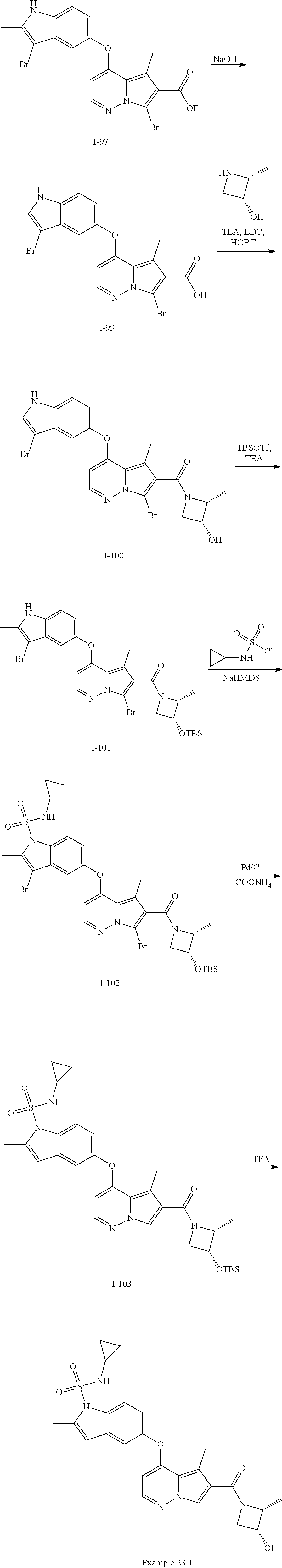

### 7-bromo-4-((3-bromo-2-methyl-1H-indol-5-yl)oxy)-5-methylpyrrolo[1,2-b]pyridazine-6-carboxylic acid (I-99)

To a solution of ethyl 7-bromo-4-((3-bromo-2-methyl-1H-indol-5-yl)oxy)-5-methylpyrrolo[1,2-b]pyridazine-6-carboxylate (2.00 g, 3.94 mmol) in THF (8.00 mL) and ethanol (8.00 mL) was added NaOH (0.631 g, 15.8 mmol) in water (4.00 mL) at 25° C. After stirring for 4 hours at 60° C., the solvent was removed under reduced pressure. The mixture was purified by mass-directed reverse phase HPLC (MeCN/water with 0.1% TFA modifier) to afford the desired product. MS: 480 [M+1]⁺.

### (7-bromo-4-((3-bromo-2-methyl-1H-indol-5-yl)oxy)-5-methylpyrrolo[1,2-b]pyridazin-6-yl)((2R,3R)-3-hydroxy-2-methylazetidin-1-yl)methanone (I-100)

To a solution of 7-bromo-4-((3-bromo-2-methyl-1H-indol-5-yl)oxy)-5-methylpyrrolo[1,2-b]pyridazine-6-carboxylic acid (200 mg, 0.417 mmol) in DMF (5.00 mL) was added (2R,3R)-2-methylazetidin-3-ol, TFA (84.0 mg, 0.417 mmol), DIPEA (0.365 mL, 2.09 mmol), HOBT (169 mg, 1.25 mmol) and EDC (240 mg, 1.25 mmol). The mixture was stirred at 25° C. for 16 hours. The reaction mixture was diluted with 5 mL EtOAc and 10 mL water. The resulting mixture was extracted with EtOAc (5 mL×3). The combined organic phases were washed with brine (10 mL), dried with Na₂SO₄, filtered and concentrated in vacuo to afford the desired product. The material was used directly in the next step without further purification. MS: 549 [M+1]⁺.

### (7-bromo-4-((3-bromo-2-methyl-1H-indol-5-yl)oxy)-5-methylpyrrolo[1,2-b]pyridazin-6-yl)((2R,3R)-3-((tert-butyldimethylsilyl)oxy)-2-methylazetidin-1-yl)methanone (I-101)

To a solution of (7-bromo-4-((3-bromo-2-methyl-1H-indol-5-yl)oxy)-5-methylpyrrolo[1,2-b]pyridazin-6-yl)((2R,3R)-3-hydroxy-2-methylazetidin-1-yl)methanone (200 mg, 0.365 mmol) and TEA (0.254 mL, 1.82 mmol) in DCM (4.00 mL) was added tert-butyldimethylsilyl trifluoromethanesulfonate (0.168 mL, 0.730 mmol) dropwise at 0° C. After stirring for 2 hours at 25° C., the reaction mixture was diluted with 5 mL DCM and 10 mL water. The resulting mixture extracted with DCM (5 mL×3). The combined organic phases were washed with brine (10 mL), dried with Na₂SO₄, filtered and concentrated in vacuo. The residue was purified by prep-TLC (petroleum ether/EtOAc=2:1) to afford the desired product. MS: 663 [M+1]⁺.

### 3-bromo-5-((7-bromo-6-((2R,3R)-3-((tert-butyldimethylsilyl)oxy)-2-methylazetidine-1-carbonyl)-5-methylpyrrolo[1,2-b]pyridazin-4-yl)oxy)-N-cyclopropyl-2-methyl-1H-indole-1-sulfonamide (I-102)

To a solution of (7-bromo-4-((3-bromo-2-methyl-1H-indol-5-yl)oxy)-5-methylpyrrolo[1,2-b]pyridazin-6-yl)((2R,3R)-3-((tert-butyldimethylsilyl)oxy)-2-methylazetidin-1-yl)methanone (100 mg, 0.151 mmol) in THF (5.00 mL) was added NaHMDS (0.755 mL, 0.755 mmol) (1 M in THF) at 0° C. over 1 min. After stirring for 0.5 hours at 0° C., cyclopropylsulfamoyl chloride (70.5 mg, 0.453 mmol) was added to the mixture at 0° C. The resulting mixture was stirred for 1 hour at 25° C. Then another NaHMDS (0.755 mL, 0.755 mmol) (1 M in THF) was added into the mixture at 0° C., followed by cyclopropylsulfamoyl chloride (70.5 mg, 0.453 mmol). The resulting mixture was stirred for another 1 hour at 25° C. The reaction mixture was diluted with 5 mL EtOAc and 10 mL water. The resulting mixture extracted with EtOAc (5 mL×3). The combined organic phases were washed with brine (10 mL), dried with Na₂SO₄, filtered and concentrated in vacuo. The residue was purified by prep-TLC (petroleum ether/EtOAc=3: 2) to afford the desired product. MS: 782 [M+1]⁺.

### 5-((6-((2R,3R)-3-((tert-butyldimethylsilyl)oxy)-2-methylazetidine-1-carbonyl)-5-methylpyrrolo[1,2-b]pyridazin-4-yl)oxy)-N-cyclopropyl-2-methyl-1H-indole-1-sulfonamide (I-13)

To a mixture of 3-bromo-5-((7-bromo-6-((2R,3R)-3-((tert-butyldimethylsilyl)oxy)-2-methylazetidine-1-carbonyl)-5-methylpyrrolo[1,2-b]pyridazin-4-yl)oxy)-N-cyclopropyl-2-methyl-1H-indole-1-sulfonamide (60.0 mg, 77.0 μmol) and ammonium formate (48.4 mg, 0.768 mmol) in DMF (1.00 mL) and water (0.200 mL) was added Pd—C (40.8 mg, 38.0 μmol) (10% wt). The mixture was stirred at 25° C. for 2 hours. After the mixture was filtered, the filtrate containing the desired product was used directly in the next step without the further purification. MS: 624 [M+1]⁺.

### N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-sulfonamide (Example 23.1)

To a mixture of 5-((6-((2R,3R)-3-((tert-butyldimethylsilyl)oxy)-2-methylazetidine-1-carbonyl)-5-methylpyrrolo[1,2-b]pyridazin-4-yl)oxy)-N-cyclopropyl-2-methyl-1H-indole-1-sulfonamide (40.0 mg, 64.0 μmol) in DMF (1.00 mL) was added TFA (1.00 mL). The mixture was stirred at 25° C. for 1 hour. The mixture was purified by mass-directed reverse phase HPLC (MeCN/water with 0.1% TFA modifier) to afford the desired product. MS: 510 [M+1]⁺. ¹H NMR (400 MHz, CD₃OD) δ 8.05 (d, J=8.8 Hz, 1H), 7.88 (s, 1H), 7.81 (d, J=5.6 Hz, 1H), 7.34 (d, J=2.0 Hz, 1H), 7.12-7.04 (m, 1H), 6.48 (s, 1H), 5.59 (d, J=5.2 Hz, 1H), 4.83-4.59 (m, 2H), 4.45-4.31 (m, 1H), 4.24-3.88 (m, 1H), 2.69 (s, 3H), 2.63 (s, 3H), 2.22-2.10 (m, 1H), 1.50-1.15 (m, 3H), 0.49-0.41 (m, 2H), 0.29-0.23 (m, 2H).

TABLE 24
Using methods similar to that outlined in the synthesis of Example 23.1, and the
appropriate starting materials, the following compounds were prepared:

| Compound | | | Exact Mass |
|---|---|---|---|
| Number | Structure | Name | [M + 1]⁺ |
| Example 23.2 | 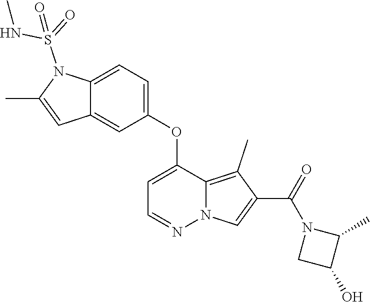 | 5-({6-[(2R,3R)-3- hydroxy-2- methylazetidine-1- carbonyl]-5- methylpyrrolo[1,2- b]pyridazin-4-yl}oxy)- N,2-dimethyl-1H-indole- 1-sulfonamide | 484 |

### Assay

The enzymatic activity of hVEGFR2 was measured using the TR-FRET based Ulight assay (Revvity) which measures phosphorylation of a fluorescently tagged peptide substrate with the Europium labeled PT66 Antibody that specifically recognizes phosphorylated tyrosine residues. The assay is performed in a buffered solution (50 mM HEPES pH 7.5, 10 mM MgCl₂, 1 mM EGTA, 0.01% Tween-20, 0.5 mM TCEP) with an ATP concentration of the measured Km of the VEGFR2 (150 M ATP). Enzyme concentrations of 50 pM VEGFR2 and Ulight peptide concentration of 50 nM Ulight-JAK1 were used. Reactions went 1 hour before being quenched with a quenching solution (buffered solution above with 20 mM EDTA and 0.5 nM Europium-anti-phosphotyrosine (PT66) Ab). The assay was further incubated for 30 minutes to allow for complete binding of the antibody and then read on a Envision plate reader to measure TR-FRET signal.

DMSO solutions of potential TKI compounds were dispensed using an ECHO 655 to dispense into White Proxi-plus 384-well plates with a total DMSO volume of 50 nl. Each compound was assessed at 10 concentrations in 3-fold serial dilutions starting at a high concentration of 2.5 M and with a low concentration of 124 pM. After compound dispensing, 2.5 μl of a 4× concentration of ATP was added to each plate well. Then 2.5 μl if a 4× concentration of the tyrosine kinase was added to each well and allowed to incubate at RT for 30 minutes. After the incubation, the kinase reaction was started with the addition of 5 μl of a 2× concentration of one of the Ulight peptides and incubated at RT for 30 or 60 minutes as indicated above. The reaction was then quenched with the addition of 5 μl of the quenching solution. Dose-response curves were generated by plotting percent inhibition (Y-axis) vs. Log 10 compound concentrations (X-axis). EC₅₀ values were calculated using a non-linear regression, four-parameters sigmoidal dose-response model.

POTENCY TABLE

| Compound | VEGFR2 EC₅₀ (nM) |
|---|---|
| Example 1.1 | 9.1 |
| Example 1.1 | 94 |
| Example 2.1 | 10 |
| Example 3.1 | 0.84 |
| Example 4.1 | 7.8 |
| Example 5.1 | 1.7 |
| Example 6.1 | 2.6 |
| Example 6.2 | 10 |
| Example 6.3 | 2.1 |
| Example 6.4 | 2.1 |
| Example 6.5 | 25 |
| Example 6.6 | 4.0 |
| Example 6.7 | 0.81 |
| Example 6.8 | 3.3 |
| Example 6.9 | 120 |
| Example 6.10 | 8.1 |
| Example 6.11 | 90 |
| Example 6.12 | 15 |
| Example 6.13 | 2.1 |
| Example 6.14 | 11 |
| Example 6.15 | 9.5 |
| Example 6.16 | 2.9 |
| Example 6.17 | 3.0 |
| Example 6.18 | 2.6 |
| Example 6.19 | 2.5 |
| Example 6.20 | 3.8 |
| Example 6.21 | 3.4 |
| Example 6.22 | 3.2 |
| Example 6.23 | 0.55 |
| Example 6.24 | 1.9 |
| Example 6.25 | 2.8 |
| Example 6.26 | 1.6 |
| Example 6.27 | 1.9 |
| Example 6.28 | 1.9 |
| Example 6.29 | 1.1 |
| Example 6.30 | 21 |
| Example 6.31 | 22 |
| Example 6.32 | 20 |
| Example 6.33 | 15 |
| Example 6.34 | 4.2 |
| Example 6.35 | 27 |
| Example 6.36 | 31 |
| Example 6.37 | 0.45 |
| Example 6.38 | 1.1 |
| Example 6.39 | 0.85 |
| Example 6.40 | 1.1 |
| Example 6.41 | 2.1 |
| Example 6.42 | 2.4 |
| Example 6.43 | 0.73 |
| Example 6.44 | 3.5 |
| Example 7.1 | 43 |
| Example 7.2 | 1.0 |
| Example 7.3 | 4.7 |
| Example 7.4 | 2.3 |
| Example 7.5 | 0.55 |
| Example 7.6 | 2.5 |
| Example 7.7 | 1.2 |
| Example 7.8 | 1.6 |
| Example 7.9 | 0.65 |
| Example 7.10 | 3.5 |
| Example 7.11 | 3.6 |
| Example 7.12 | 3.2 |
| Example 7.13 | 4.7 |
| Example 7.14 | 0.80 |
| Example 7.15 | 6.0 |
| Example 7.16 | 4.3 |
| Example 7.17 | 2.3 |
| Example 7.18 | 1.1 |
| Example 7.19 | 2.9 |
| Example 7.20 | 6.2 |
| Example 7.21 | 5.5 |
| Example 7.22 | 15 |
| Example 7.23 | 100 |
| Example 7.24 | 2.1 |
| Example 7.25 | 2.3 |
| Example 7.26 | 4.3 |
| Example 7.27 | 4.3 |
| Example 7.28 | 1.3 |
| Example 7.29 | 2.4 |
| Example 7.30 | 1.5 |
| Example 7.31 | 5.8 |
| Example 7.32 | 4.9 |
| Example 7.33 | 6.0 |
| Example 7.34 | 5.0 |
| Example 7.35 | 4.2 |
| Example 7.36 | 5.9 |
| Example 7.37 | 5.6 |
| Example 7.38 | 6.1 |
| Example 7.39 | 5.1 |
| Example 7.40 | 40 |
| Example 7.41 | 7.1 |
| Example 7.42 | 9.6 |
| Example 7.43 | 5.3 |
| Example 7.44 | 8.2 |
| Example 7.45 | 12 |
| Example 7.46 | 5.8 |
| Example 7.47 | 18 |
| Example 7.48 | 5.6 |
| Example 7.49 | 7.6 |
| Example 7.50 | 10 |
| Example 7.51 | 5.6 |
| Example 7.52 | 8.3 |
| Example 7.53 | 6.0 |
| Example 7.54 | 9.0 |
| Example 7.55 | 6.7 |
| Example 7.56 | 5.2 |
| Example 7.57 | 14 |
| Example 7.58 | 6.5 |
| Example 7.59 | 3.7 |
| Example 7.60 | 3.0 |
| Example 7.61 | 9.7 |
| Example 7.62 | 6.1 |
| Example 7.63 | 12 |
| Example 7.64 | 4.4 |
| Example 7.65 | 3.9 |
| Example 7.66 | 1.5 |
| Example 7.67 | 6.5 |
| Example 7.68 | 1.1 |
| Example 7.69 | 1.3 |
| Example 7.70 | 2.1 |
| Example 7.71 | 1.0 |
| Example 7.72 | 5.1 |
| Example 7.73 | 5.7 |
| Example 7.74 | 2.3 |
| Example 7.75 | 320 |
| Example 7.76 | 44 |
| Example 7.77 | 12 |
| Example 7.78 | 4.5 |
| Example 7.79 | 1.6 |
| Example 7.80 | 1.5 |
| Example 7.81 | 2.8 |
| Example 7.82 | 2.9 |
| Example 7.83 | 5.1 |
| Example 7.84 | 1.8 |
| Example 7.85 | 4.5 |
| Example 7.86 | 3.0 |
| Example 7.87 | 6.1 |
| Example 7.88 | 2.2 |
| Example 7.89 | 11 |
| Example 7.90 | 12 |
| Example 7.91 | 2.1 |
| Example 7.92 | 8.8 |
| Example 7.93 | 11 |
| Example 7.94 | 1.9 |
| Example 7.95 | 32 |
| Example 7.96 | 20 |
| Example 7.97 | 2.4 |
| Example 7.98 | 5.7 |
| Example 7.99 | 6.2 |
| Example 7.100 | 4.6 |
| Example 7.101 | 3.8 |
| Example 7.102 | 5.9 |
| Example 7.103 | 11 |
| Example 7.104 | 3.6 |
| Example 7.105 | 2.9 |
| Example 7.106 | 5.0 |
| Example 7.107 | 16 |
| Example 7.108 | 1.2 |
| Example 7.109 | 1.1 |
| Example 7.110 | 6.2 |
| Example 7.111 | 3.6 |
| Example 7.112 | 1.4 |
| Example 7.113 | 2.0 |
| Example 7.114 | 22 |
| Example 7.115 | 42 |
| Example 7.116 | 15 |
| Example 7.117 | 19 |
| Example 7.118 | 2.5 |
| Example 7.119 | 2.0 |
| Example 7.120 | 12 |
| Example 7.121 | 11 |
| Example 7.122 | 2.5 |
| Example 7.123 | 3.0 |
| Example 7.124 | 34 |
| Example 7.125 | 73 |
| Example 7.126 | 24 |
| Example 7.127 | 31 |
| Example 7.128 | 3.2 |
| Example 7.129 | 61 |
| Example 7.130 | 34 |
| Example 7.131 | 22 |
| Example 7.132 | 8.7 |
| Example 7.133 | 5.9 |
| Example 7.134 | 7.9 |
| Example 7.135 | 12 |
| Example 7.136 | 130 |
| Example 7.137 | 51 |
| Example 7.138 | 37 |
| Example 7.139 | 48 |
| Example 7.140 | 71 |
| Example 7.141 | 3.5 |
| Example 7.142 | 31 |
| Example 7.143 | 6.2 |
| Example 7.144 | 74 |
| Example 7.145 | 42 |
| Example 7.146 | 10 |
| Example 7.147 | 18 |
| Example 7.148 | 34 |
| Example 7.149 | 150 |
| Example 7.150 | 70 |
| Example 7.151 | 110 |
| Example 7.152 | 660 |
| Example 7.153 | 1.2 |
| Example 7.154 | 2.2 |
| Example 7.155 | 3.8 |
| Example 7.156 | 0.55 |
| Example 7.157 | 3.1 |
| Example 7.158 | 0.85 |
| Example 7.159 | 0.80 |
| Example 7.160 | 0.85 |
| Example 7.161 | 0.71 |
| Example 7.162 | 3.2 |
| Example 8.1 | 5.4 |
| Example 8.2 | 5.2 |
| Example 8.3 | 2.7 |
| Example 8.4 | 19 |
| Example 8.5 | 6.1 |
| Example 8.6 | 6.6 |
| Example 8.7 | 10 |
| Example 8.8 | 45 |
| Example 8.9 | 12 |
| Example 8.10 | 25 |
| Example 8.11 | 9.8 |
| Example 8.12 | 2.0 |
| Example 8.13 | 4.3 |
| Example 8.14 | 5.9 |
| Example 8.15 | 9.4 |
| Example 8.16 | 37 |
| Example 8.17 | 10 |
| Example 8.18 | 22 |
| Example 8.19 | 63 |
| Example 8.20 | 12 |
| Example 8.21 | 9.2 |
| Example 8.22 | 11 |
| Example 8.23 | 3.2 |
| Example 8.24 | 4.7 |
| Example 8.25 | 6.1 |
| Example 8.26 | 11 |
| Example 8.27 | 10 |
| Example 8.28 | 110 |
| Example 8.29 | 70 |
| Example 8.30 | 37 |
| Example 8.31 | 68 |
| Example 8.32 | 91 |
| Example 8.33 | 33 |
| Example 8.34 | 140 |
| Example 8.35 | 57 |
| Example 8.36 | 31 |
| Example 8.37 | 340 |
| Example 8.38 | 77 |
| Example 8.39 | 5.6 |
| Example 8.40 | 31 |
| Example 8.41 | 66 |
| Example 8.42 | 32 |
| Example 8.43 | 8.6 |
| Example 8.44 | 22 |
| Example 8.45 | 22 |
| Example 8.46 | 20 |
| Example 8.47 | 4.4 |
| Example 8.48 | 6.8 |
| Example 8.49 | 7.1 |
| Example 8.50 | 6.6 |
| Example 8.51 | 12 |
| Example 8.52 | 8.9 |
| Example 8.53 | 7.0 |
| Example 8.54 | 8.6 |
| Example 8.55 | 4.1 |
| Example 8.56 | 9.4 |
| Example 8.57 | 34 |
| Example 8.58 | 20 |
| Example 8.59 | 54 |
| Example 8.60 | 29 |
| Example 9.1A | 26 |
| Example 9.1B | 7.5 |
| Example 9.2A | 34 |
| Example 9.2B | 21 |
| Example 9.3A | 1.6 |
| Example 9.3B | 1.3 |
| Example 10.1A | 3.3 |
| Example 10.1B | 3.1 |
| Example 10.2A | 2.8 |
| Example 10.2B | 1.7 |
| Example 10.3A | 2.7 |
| Example 10.3B | 9.2 |
| Example 10.4A | 12 |
| Example 10.5A | 14 |
| Example 11.1A | 3.9 |
| Example 11.1B | 3.6 |
| Example 11.2A | 6.0 |
| Example 11.2B | 8.5 |
| Example 12.1A | 7.5 |
| Example 12.1B | 0.8 |
| Example 13.1 | 2.2 |
| Example 13.2 | 31 |
| Example 13.3 | 1.1 |
| Example 13.4 | 2.2 |
| Example 13.5 | 140 |
| Example 13.6 | 180 |
| Example 13.7 | 249 |
| Example 13.8 | 72 |
| Example 13.9 | 79 |
| Example 13.10 | 210 |
| Example 13.11 | 150 |
| Example 13.12 | 190 |
| Example 13.13 | 140 |
| Example 13.14 | 300 |
| Example 13.15 | 18 |
| Example 13.16 | 15 |
| Example 13.17 | 610 |
| Example 13.18 | 330 |
| Example 14.1 | 0.49 |
| Example 14.2 | 0.71 |
| Example 14.3 | 7.5 |
| Example 14.4 | 370 |
| Example 14.5 | 320 |
| Example 14.6 | 260 |
| Example 14.7 | 270 |
| Example 14.8 | 3.6 |
| Example 15.1 | 19 |
| Example 15.2 | 39 |
| Example 15.3 | 150 |
| Example 15.4 | 83 |
| Example 16.1 | 3.0 |
| Example 16.2 | 1.2 |
| Example 16.3 | 3.8 |
| Example 16.4 | 340 |
| Example 16.5 | 4.8 |
| Example 16.6 | 7.7 |
| Example 16.7 | 12 |
| Example 16.8 | 29 |
| Example 16.9 | 16 |
| Example 16.10 | 2.7 |
| Example 16.11 | 14 |
| Example 16.12 | 9.7 |
| Example 16.13 | 53 |
| Example 16.14 | 8.8 |
| Example 16.15 | 5.1 |
| Example 16.16 | 6.3 |
| Example 16.17 | 6.6 |
| Example 16.18 | 25 |
| Example 16.19 | 5.2 |
| Example 16.20 | 60 |
| Example 16.21 | 17 |
| Example 16.22 | 7.3 |
| Example 16.23 | 140 |
| Example 16.24 | 30 |
| Example 16.25 | 78 |
| Example 16.26 | 18 |
| Example 16.27 | 108 |
| Example 16.28 | 630 |
| Example 16.29 | 12 |
| Example 16.30 | 39 |
| Example 16.31 | 1.4 |
| Example 16.32 | 13 |
| Example 16.33 | 2.7 |
| Example 16.34 | 64 |
| Example 16.35 | 28 |
| Example 16.36 | 4.3 |
| Example 16.37 | 3.1 |
| Example 16.38 | 0.75 |
| Example 16.39 | 8.4 |
| Example 16.40 | 4.4 |
| Example 16.41 | 5.0 |
| Example 16.42 | 8.9 |
| Example 16.43 | 1.6 |
| Example 16.44 | 8.0 |
| Example 16.45 | 1.5 |
| Example 17.1 | 7.7 |
| Example 17.2 | 14 |
| Example 17.3 | 4.5 |
| Example 17.4 | 18 |
| Example 17.5 | 71 |
| Example 18.1 | 170 |
| Example 18.2 | 38 |
| Example 18.3 | 17 |
| Example 19.1 | 13 |
| Example 19.2 | 13 |
| Example 20.1 | 0.49 |
| Example 20.2 | 0.63 |
| Example 21.1 | 0.35 |
| Example 22.1 | 150 |
| Example 23.1 | 9.8 |
| Example 23.2 | 32 |

What is claimed is:

1. A compound of Formula I: 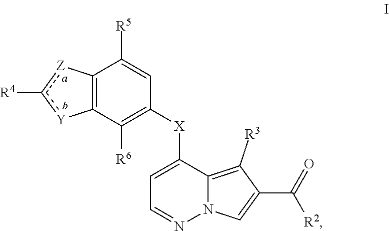
or a pharmaceutically acceptable salt thereof, wherein
bonds “a” and “b” represent double bonds “- - -” wherein: “a” and “b” are single bonds;
“a” is a single bond and “b” is a double bond, or “a” is a double bond and “b” is a single bond;
X is selected from —O—, —NH—, —N—C₁₋₆alkyl-, and —S—;
Y is selected from —O—, —S—, —N—, —C—R¹—, —C(H)—R¹—, —NR¹— and —CH₂—;
Z is selected from —C—R¹, —C(H)—R¹, —NR¹—, —S—, —N— and —O—;
when present, R¹ is selected from:
(1) —C₁₋₆alkyl,
(2) halo,
(3) —C₁₋₆alkyl-(O)NHR^{a},
(4) —NHC(O)NHR^{a},₂
(5) —NHC(O)R^{a},
(6) —C₀₋₆alkyl-OR^{a},
(7) —C₀₋₆alkyl-C(O)OR^{a},
(8) —C₀₋₆alkyl-C(O)R^{a},
(9) —C₀₋₆alkyl-cyano,
(10) —C₀₋₆alkyl-NHR^{a},
(11) —C₀₋₆alkyl-aryl, wherein the aryl is unsubstituted or substituted with 1-3 substituents independently selected from R^{a},
(12) —C₀₋₆alkyl-(5-6 membered heteroaryl) wherein the heteroaryl has 1-3 heteroatoms independently selected from N, O, and S, and is unsubstituted or substituted with 1-3 substituents independently selected from R^{a},
(13) —C₃₋₆cycloalkyl, unsubstituted or substituted with 1-3 substituents selected from R^{a},
(14) —C₀₋₆alkyl-C(O)NHR^{a},
(15) —C₀₋₆alkyl-C(O)NR⁷R⁸,
(16) —C₀₋₆alkyl-S(O)₂NHR^{a}, and
(17) hydrogen;
when present, R^{a} is selected from:
(1) hydrogen,
(2) —C₁₋₆alkyl, unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, and —C₁₋₆haloalkyl,
(3) —C₀₋₆alkyl-C₃₋₆cycloalkyl, unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, —C(O)—C₁₋₆alkyl, and —C₁₋₆haloalkyl,
(4) —C₀₋₆alkyl-aryl, unsubstituted or substituted with 1-3 substituents independently selected from halo, ═O, —NH-cyclopropyl, —OH, —C₀₋₆alkyl-NR⁷R⁸, —C₀₋₆alkyl-cyano, —C₁₋₆alkyl, —O—C₀₋₆alkyl, and —C₁₋₆haloalkyl,
(5) —O—C₁₋₆alkyl,
(6) —C₀₋₆alkyl-(5-6 membered monocyclic heteroaryl) wherein the heteroaryl has 1-3 heteroatoms independently selected from N, O, and S, and is unsubstituted or substituted with 1-3 substituents independently selected from halo, ═O, —NH-cyclopropyl, —OH, —C₀₋₆alkyl-NR⁷R⁸, —C₀₋₆alkyl-cyano, —C₁₋₆alkyl, —O—C₀₋₆alkyl, and —C₁₋₆haloalkyl,
(7) —C₁₋₆alkyl-S—C₁₋₆alkyl,
(8) —C₃₋₆ monocyclic heterocyclyl having 1-3 ring heteroatoms independently selected from N, O, and S, unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, —C₁₋₆haloalkyl, and ═O,
(9) 5-10 membered spiro bicyclic or tricyclic cycloalkyl, unsubstituted or substituted with 1-3 substituents independently selected from halo, —OH, —C₀₋₆alkyl-NR⁷R⁸, —C₀₋₆alkyl-cyano, —C₁₋₆alkyl, —O C₀₋₆alkyl, —C₁₋₆haloalkyl, and ═O,
(10) 5-10 membered spiro bicyclic or tricyclic heterocyclyl having 1-3 ring heteroatoms independently selected from N, O, and S, wherein the spiro bicyclic or tricyclic heterocyclyl ring is unsubstituted or substituted with 1-3 substituents independently selected from halo, —OH, —C₀₋₆alkyl-NR⁷R⁸, —C₀₋₆alkyl-cyano, —C₁₋₆alkyl, —O—C₀₋₆alkyl, —C₁₋₆haloalkyl, and ═O,
(11) 5-6 membered bridged bicyclic cycloalkyl, unsubstituted or substituted with 1 substituent selected from —OH, halo and haloalkyl,
(12) halo,
(13) —C(O)O—C₁₋₆alkyl, and
(14) —NH₂;
when present, each of R⁷ and R¹ is independently selected from
(1) hydrogen,
(2) —C₀₋₆alkyl-(3-6 membered heterocyclyl), wherein the heterocylyl has 1-3 ring heteroatoms selected from N, O, and S, and is unsubstituted or substituted with 1-3 substituents independently selected from R^{b},
(3) —C₁₋₆alkyl, unsubstituted or substituted with 1-3 substituents independently selected from R^{b},
(4) —C₁₋₆ alkyl-O—C₁₋₆alkyl, unsubstituted or substituted with 1-3 substituents independently selected from R^{b},
(5) —C₀₋₆alkyl-C₃₋₆ cycloalkyl, wherein the cycloalkyl is unsubstituted or substituted with 1-3 substituents independently selected from R^{b}, and
(6) 5-10 membered bicyclic or tricyclic cycloalkyl, wherein the cycloalkyl is unsubstituted or substituted with 1-3 substituents independently selected from R^{b};
when present, R^{b} is selected from
(1) —C₁₋₆alkyl,
(2) halo,
(3) —NH₂,
(4) ═O, and
(5) —OH;
or alternatively, R⁷ and R⁸ together with the nitrogen to which they are attached form 3-10 membered heterocyclyl having 0-3 ring heteroatoms independently selected from N, O, and S, in addition to the nitrogen to which R⁷ and R⁸ are attached, wherein the heterocyclyl is optionally substituted with 1-4 substituents independently selected from R^{c};
when present, R^{c} is selected from
(1) —C₀₋₆alkyl-OH,
(2) —C₁₋₆alkyl, unsubstituted or substituted with 1-3 substituents independently selected from —OH,
(3) —C₀₋₆alkyl-cyano,
(4) —C₀₋₆alkyl-O—C₁₋₃alkyl,
(5) —C₀₋₃alkyl-(5-6 membered monocyclic heteroaryl), wherein the heteroaryl has 1-3 heteroatoms selected from N, O, and S, and is unsubstituted or substituted with 1-3 substituents independently selected from C₁₋₆alkyl,
(6) halo,
(7) —C₁₋₆haloalkyl,
(8) C₀₋₆alkyl-aryl, wherein the aryl is unsubstituted or substituted with 1-3 substituents independently selected from C₁₋₆haloalkyl, and halo,
(9) —C₃₋₆cycloalkyl,
(10) —C₀₋₆alkyl-N, unsubstituted or substituted with 1-3 substituents independently selected from hydrogen and C₁₋₆alkyl,
(11) —NH—C(O)C₁₋₆alkyl,
(12) —C₀₋₃alkyl-(3-6 membered monocyclic heterocyclyl), wherein the heterocyclyl has 1-3 ring heteroatoms selected from N, O, and S, and is unsubstituted or substituted with 1 ═O substituent,
(13) —O—C₁₋₆haloalkyl,
(14) ═O,
(15) hydrogen, and
(16) —C(O)OC₁₋₆alkyl;
R² is selected from
(1) —O—C₁₋₆alkyl, and 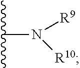
each of R⁹ and R¹⁰ is independently selected from
(1) hydrogen,
(2) —C₀₋₆alkyl-(3-6 membered heterocyclyl), wherein the heterocyclyl has 1-3 ring heteroatoms selected from N, O, and S, and the is unsubstituted or substituted with 1-3 substituents independently selected from R^{d},
(3) —C₁₋₆alkyl, unsubstituted or substituted with 1-3 substituents independently selected from R^{d},
(4) —C₁₋₆ alkyl-O—C₁₋₆alkyl, unsubstituted or substituted with 1-3 substituents independently selected from R^{d},
(5) —C₀₋₆alkyl-C₃₋₆ cycloalkyl, wherein the cycloalkyl is unsubstituted or substituted with 1-3 substituents independently selected from R^{d}, and
(6) 5-10 membered bicyclic or tricyclic cycloalkyl, wherein the cycloalkyl is unsubstituted or substituted with 1-3 substituents independently selected from R^{d};
When present, R^{d} is selected from
(1) —C₁₋₆alkyl,
(2) halo,
(3) —NH₂,
(4) ═O, and
(5) —OH;
or alternatively, R⁹ and R¹⁰ together with the nitrogen to which they are attached form 4-10 membered heterocyclyl having 0-3 ring heteroatoms independently selected from N, O, and S, in addition to the nitrogen to which R⁹ and R¹⁰ are attached, wherein the heterocyclyl is optionally substituted with 1-4 substituents independently selected from R^{e};
when present, R^{e} is selected from
(1) —C₀₋₆alkyl-OH,
(2) —C₁₋₆alkyl, unsubstituted or substituted with 1-3 substituents independently selected from —OH,
(3) —C₀₋₆alkyl-cyano,
(4) —C₀₋₆alkyl-O—C₁₋₃alkyl,
(5) —C₀₋₃alkyl-(5-6 membered monocyclic heteroaryl), wherein the heteroaryl has 1-3 ring heteroatoms selected from N, O, and S, and is unsubstituted or substituted with 1-3 substituents independently selected from C₁₋₆alkyl,
(6) halo,
(7) —C₁₋₆haloalkyl,
(8) C₀₋₆alkyl-aryl, wherein the aryl is unsubstituted or substituted with 1-3 substituents independently selected from C₁₋₆haloalkyl, and halo,
(9) —C₃₋₆cycloalkyl,
(10) —C₀₋₆alkyl-N(R^{d})₂, wherein each R^{d} is independently selected from hydrogen and C₁₋₆alkyl,
(11) —NH—C(O)C₁₋₆alkyl,
(12) —C₀₋₃alkyl-(3-6 membered monocyclic heterocyclyl), wherein the heterocyclyl has 1-3 ring heteroatoms selected from N, O, and S, and is unsubstituted or substituted with 1 ═O substituent,
(13) —O—C₁₋₆haloalkyl,
(14) ═O,
(15) hydrogen, and
(16) —C(O)OC₁₋₆alkyl;
R³ is selected from
(1) hydrogen,
(2) —C₁₋₆alkyl,
(3) —C₃₋₆cycloalkyl,
(4) —C₁₋₆haloalkyl,
(5) —C₁₋₆alkyl-O—C₁₋₆alkyl, and
(6) —C₁₋₆alkyl-OH;
R⁴ is selected from
(1) hydrogen,
(2) halogen,
(3) —C₁₋₆alkyl,
(4) —C(O)NH—C₀₋₆alkyl,
(5) —C₀₋₆alkyl-(O)OC₁₋₆alkyl,
(6) —NH₂
(7) —C₁₋₆haloalkyl,
(8) —C(O)NH₂, and
(9) —C₃₋₆cycloalkyl;
R⁵ is selected from
(1) hydrogen,
(2) halogen,
(3) —C₁₋₆alkyl, and
(4) C₁₋₆alkyl-(O)OC₁₋₆alkyl;
R⁶ is selected from
(1) hydrogen,
(2) halogen, and
(3) —C₁₋₆alkyl.

2. The compound of claim 1, or a pharmaceutically acceptable salt thereof, wherein R¹ is
(1) —C₁₋₅alkyl,
(2) —C₁₋₃alkyl-(O)NH—C₀₋₃alkyl, unsubstituted or substituted with 1-3 substituents independently selected from: cyclobutanyl, unsubstituted or substituted with 1-3 substituents independently selected from halo, —C₁₋₃alkyl-haloalkyl, —C₁₋₃alkyl, —O—C₁₋₃alkyl, —OH, and C₀₋₁alkyl-cyano; bicyclo[1.1.1]pentanyl, unsubstituted or substituted with 1-3 substituents independently selected from C₁₋₃haloalkyl, —OH and —F; cyclopropanyl, unsubstituted or substituted with 1-3 substituents independently selected from —C₀₋₁alkyl-cyano, —C₁₋₃alkyl, —C₁₋₃ haloalkyl, and —F; phenyl unsubstituted or substituted with 1-3 substituents independently selected from methyl or —F; cyclopentanyl, unsubstituted or substituted with 1-3 substituents independently selected from —F; spiro[3.3]heptanyl; spiro[2.3]hexanyl, unsubstituted or substituted with 1 —F substituent; oxaspiro[3.3]heptanyl; thietanyl, substituted with 1 ═O substituent; azetidinyl, unsubstituted or substituted with 1 —C₁₋₃haloalkyl substituent; tetrahydrofuranyl, unsubstituted or substituted with 1 —C₁₋₃alkyl substituent; azetidinyl, substituted with 1 —C₁₋₃haloalkyl substituent; —OH; —C₀₋₃alkyl-C(O)NH₂; and oxetanyl, unsubstituted or substituted with 1 methyl substituent,
(3) —C₀₋₃alkyl-C(O)O—C₁₋₃alkyl,
(4) —C₀₋₃alkyl-C(O)-heterocyclyl, wherein the heterocyclyl is unsubstituted or substituted with 1 halo substituent,
(5) —SO₂NH—C₃₋₆cycloalkyl, or —SO₂NH—C₁₋₃alkyl, or
(6) hydrogen.

3. The compound of claim 2, or a pharmaceutically acceptable salt thereof, wherein R¹ is
(1) —C₁₋₃alkyl,
(2) —C(O)NH—C₀₋₁alkyl-cyclobutanyl, unsubstituted or substituted with 1-2 substituents independently selected from —F, —CF₃, C₁₋₃alkyl, —OCH₃, —OH and C₀₋₁alkyl-cyano; —C(O)NH-bicyclo[1.1.1]pentanyl, unsubstituted or substituted with 1 substituent selected from —OH, —F and —CF₃; —C(O)NH—C₀₋₁alkyl-cyclopropanyl, unsubstituted or substituted with 1-2 —F substituents; —C(O)NH—C₀₋₁alkyl-phenyl; —C(O)NH—C₁₋₃alkyl, unsubstituted or substituted with 1 —OH substituent; —C(O)NH—C₀₋₁alkyl-cyclopentanyl; —C(O)NH-spiro[3.3]heptanyl; —C(O)NH-spiro[2.3]hexanyl, unsubstituted or substituted with 1 —F substituent; —C(O)NH-oxaspiro[3.3]heptanyl; —C(O)NH-thietane, substituted with 1 ═O substituent; tetrahydrofuranyl, unsubstituted or substituted with 1 methyl substituent; azetidinyl, substituted with 1 —CH₂CF₃ substituent; or —C(O)NH-oxetanyl, unsubstituted or substituted with 1 methyl substituent,
(3) —C(O)OCH₃ or —C(O)OCH₂CH₃,
(4) —C(O)-azetidinyl, unsubstituted or substituted with one —F substituent,
(5) —SO₂NH-cyclopropyl, or —SO₂NHCH₃, or
(5) hydrogen.

4. The compound of claim 3, or a pharmaceutically acceptable salt thereof, wherein R¹ is —H, —C₁₋₃alkyl,

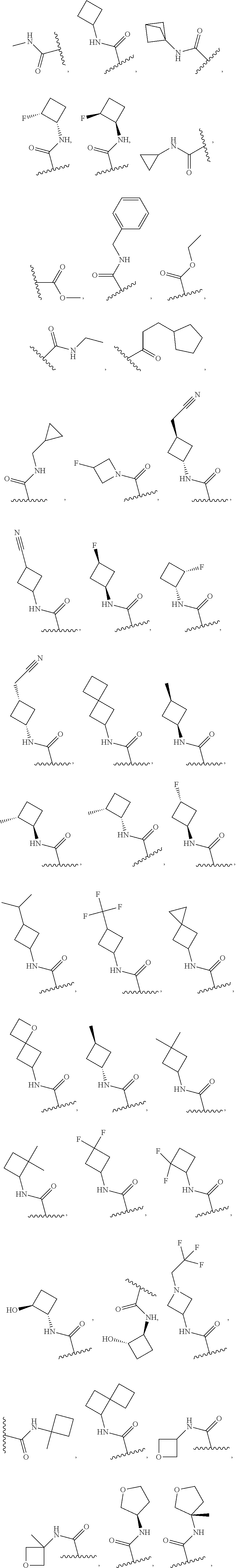

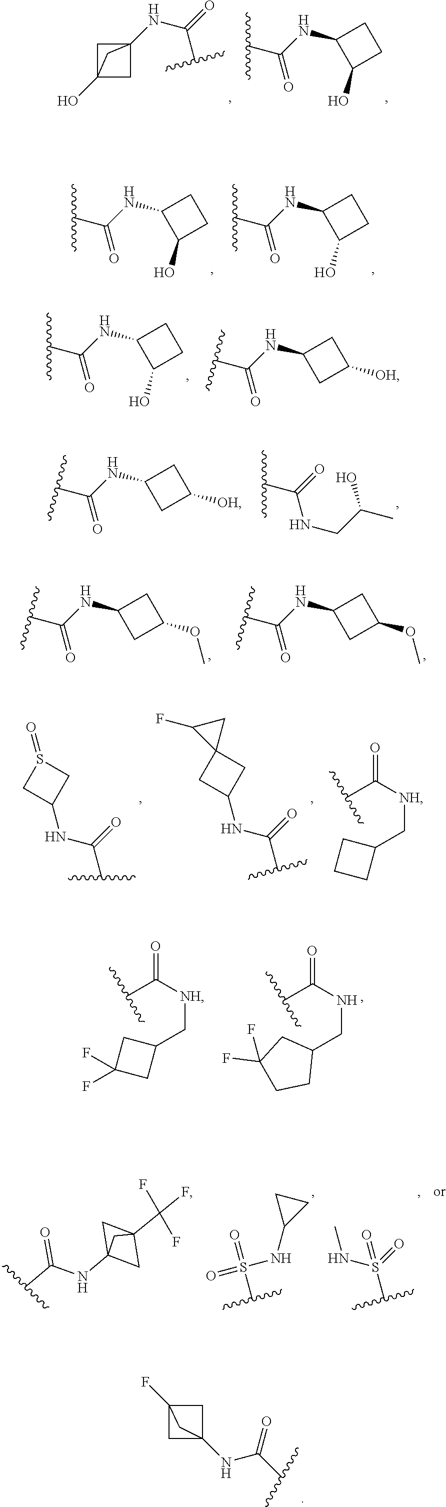

5. The compound of claim 1, or a pharmaceutically acceptable salt thereof, wherein R^{a} is
(1) hydrogen,
(2) —C₁₋₃alkyl,
(3) —C₀₋₃alkyl-C₃₋₆cycloalkyl, unsubstituted or substituted with 1-3 substituents independently selected from halo, —C₁₋₂alkyl-cyano, —C₁₋₆alkyl, —C₁₋₆alkyl-O—C₀₋₆alkyl, and —C₁₋₆haloalkyl,
(4) —C₀₋₆alkyl-aryl, unsubstituted or substituted with 1-3 substituents independently selected from halo, ═O, and —NH—C₃₋₆cycloalkyl,
(5) —O—C₁₋₆alkyl,
(6) 5-6 membered monocyclic heteroaryl having 1-3 ring heteroatoms independently selected from N and S, unsubstituted or substituted with 1-3 substituents independently selected from —C₁₋₆alkyl,
(7) —C₁₋₃alkyl-S—C₁₋₃alkyl,
(8) —C₃₋₆ monocyclic heterocyclyl having 1 ring heteroatom selected from N and S unsubstituted or substituted with 1-3 substituents independently selected from halo and ═O,
(9) 5-10 membered spiro bicyclic cycloalkyl, unsubstituted or substituted with 1-3 halo substituents,
(10) 5-10 membered spiro bicyclic heterocyclyl having 1 ring heteroatom selected from N, O, and S,
(11) 5-6 membered bridged bicyclic cycloalkyl, unsubstituted or substituted with 1 substituent selected from halo and C₁₋₃haloalkyl,
(12) halo,
(13) —C(O)O—C₁₋₃alkyl, or
(14) —NH₂.

6. The compound of claim 5, or a pharmaceutically acceptable salt thereof, wherein R^{a} is
(1) hydrogen,
(2) —C₁₋₃alkyl,
(3) —C₀₋₃alkyl-C₃₋₆cycloalkyl, unsubstituted or substituted with 1-3 substituents independently selected from halo, —C₁₋₂alkyl-cyano, —C₁₋₆alkyl, —C₁₋₆alkyl-O—C₀₋₆alkyl, and —C₁₋₆haloalkyl,
(4) —C₀₋₆alkyl-aryl, unsubstituted or substituted with 1-3 substituents independently selected from halo, ═O, and —NH—C₃₋₆cycloalkyl,
(5) —O—C₁₋₆alkyl,
(6) 5-6 membered monocyclic heteroaryl having 1-3 ring heteroatoms independently selected from N and S, unsubstituted or substituted with 1-3 —C₁₋₆alkyl substituents,
(7) —C₁₋₃alkyl-S—C₁₋₃alkyl,
(8) —C₃₋₆ monocyclic heterocyclyl having 1 ring heteroatom selected from N and S, wherein the monocyclic heterocyclyl is unsubstituted or substituted with 1-3 substituents independently selected from halo and ═O,
(9) 5-10 membered spiro bicyclic cycloalkyl, unsubstituted or substituted with 1-3 substituents selected from halo,
(10) 5-10 membered spiro bicyclic heterocyclyl having 1 ring heteroatom selected from N, O, and S,
(11) 5-6 membered bridged bicyclic cycloalkyl, unsubstituted or substituted with 1 substituent selected from halo and C₁₋₃haloalkyl,
(12) halo,
(13) —C(O)O—C₁₋₃alkyl, or
(14) —NH₂.

7. The compound of claim 1, or a pharmaceutically acceptable salt thereof, wherein R² is
(1) —OC₁₋₂alkyl,
(2) —NH—C₀₋₃alkyl-3-6 membered heterocyclyl, wherein the heterocyclyl has 1 ring heteroatom selected from S and O, and is unsubstituted or substituted with 1-3 substituents independently selected from —C₁₋₆alkyl, halo, ═O, and —OH,
(3) —NH—C₁₋₅alkyl, unsubstituted or substituted with 1-3 substituents independently selected from —C₁₋₆alkyl, halo, ═O, and —OH,
(4) —NH—C₀₋₃alkyl-C₃₋₆cycloalkyl, unsubstituted or substituted with 1-3 substituents independently selected from —C₁₋₆alkyl, halo, ═O, and —OH,
(5) 3-7 membered monocyclic heterocyclyl having 0-3 ring heteroatoms independently selected from N, O, and S, in addition to the nitrogen to which R⁹ and R¹⁰ are attached, wherein the monocyclic heterocyclyl is unsubstituted or substituted with 1-4 substituents independently selected from: —C₁₋₆alkyl, unsubstituted or substituted with 1-3 substituents selected from —OH; —NH₂; —C₀₋₆alkyl-cyano; —C₀₋₆alkyl-O—C₁₋₃alkyl; —C₀₋₃alkyl-5-6 membered monocyclic heteroaryl having 1-3 ring heteroatoms independently selected from N, O, and S, unsubstituted or substituted with 1-3 C₁₋₆alkyl substituents; halo; —OH; —C₁₋₆haloalkyl; C₀₋₆alkyl-aryl, wherein the aryl is unsubstituted or substituted with 1-3 substituents independently selected from C₁₋₆haloalkyl and halo; —C₃₋₆cycloalkyl; —C₀₋₆alkyl-N, unsubstituted or substituted with 1-3 substituents independently selected from hydrogen and —C₁₋₆alkyl; —NH—C(O)C₁₋₆alkyl; —N(CH₃)—C(O)C₁₋₆alkyl; and —C₀₋₃alkyl-3-6 membered monocyclic heterocyclyl having 1-3 ring heteroatoms independently selected from N, O, and S, unsubstituted or substituted with 1-3 ═O substituents,
(6) 5-10 membered spiro bicyclic heterocyclyl having 0-3 ring heteroatoms independently selected from N, O, and S, in addition to the nitrogen to which R⁹ and R¹⁰ are attached, wherein the spiro bicyclic heterocyclyl is unsubstituted or substituted with 1-3 substituents independently selected from cyano, ═O, —OH, and —C₁₋₆alkyl,
(7) 5-10 membered bridged heterocyclyl having 0-3 ring heteroatoms independently selected from N, O, and S, in addition to the nitrogen to which R⁹ and R¹⁰ are attached, wherein the bridged heterocyclyl is unsubstituted or substituted with 1-3 substituents independently selected from methyl, —C₀₋₆alkyl-OH, and hydrogen, or
(8) 5-10 membered fused bicyclic heterocyclyl having 0-3 ring heteroatoms independently selected from N, O, and S, in addition to the nitrogen to which R⁹ and R¹⁰ are attached, wherein the fused bicyclic heterocyclyl is unsubstituted or substituted with 1-3 substituents independently selected from halo, hydrogen, —C(O)OC₁₋₆alkyl, —O—C₁₋₆alkyl, —OH, and —O—C₁₋₆haloalkyl.

8. The compound of claim 7, or a pharmaceutically acceptable salt thereof, wherein R² is
(1) —OCH₃ or —OCH₂CH₃,
(2) —NH-thietanyl, substituted with 2 ═O substituents and 1 methyl substituent; —NH— tetrahydrothiopyranyl, substituted with 2 ═O substituents and 1 methyl substituent; or —N(CH₃)CH₂-pyrrolidinyl, wherein the pyrrolidinyl is substituted 2 substituents independently selected from methyl and ═O,
(3) —NH—CH₂C(CH₃)₂CH₂OCH₃,
(4) —NH-cyclohexnanyl, substituted with 1-3 substituents independently selected from —OH and —F; —N(CH₃)-cyclohexanyl substituted with 1 —OH substituent; or —NHCH(CH₃)-cyclopropanyl,
(5) pyrrolidinyl, unsubstituted or substituted with 1-3 substituents independently selected from —OH; pyrazolyl, unsubstituted or substituted with methyl; —CH₂OCH₃; —CH₂-cyano; —N(CH₃)C(O)CH₃; —CH₂-triazolyl, —CHF₂, pyridinyl; —C₁₋₄alkyl; C₀₋₁alkyl-phenyl, unsubstituted or substituted with 1 substituent selected from —C₁, —F —CF₃; —OCH₃; cyclopropyl; oxadiazolyl, substituted with 1 methyl substituent; imidazole, substituted with 1 methyl substituent; or —CH₂N(CH₃)₂,
(6) piperidinyl, substituted with 1-2 substituents independently selected from —OH, methyl, and ethyl,
(7) azetidinyl, unsubstituted or substituted with 1-3 substituents independently selected from —C₀₋₃alkyl-cyano, —OCH₃, pyrazole, —CH₂-triazole, —OH, methyl, ethyl, —CH(CH₃)₂, —CH₂OCH₃, —F, —CHF₂, —CF₃, —F, —C(CH₃)₂OH, —NH₂, cyclopropyl, —CH₂N(CH₃)₂, and —NHC(O)CH₃; or prrolidinyl, substituted with 1 ═O substituent,
(8) morpholinyl, substituted with 1 substituent selected from —CH(CH₃)₂ and methyl,
(9) azepanyl, unsubstituted or substituted with 1 —OH substituent,
(10) oxazepanyl,
(11) oxa-azaspiro[3.4]octanyl, unsubstituted or substituted with 1-2 substituents independently selected from methyl and —OH; diazaspiro[3.4]octanyl, substituted with 1 ═O substituent and 1 hydrogen substituent; azaspiro[3.3]heptanyl, substituted with 1 substituent selected from —OH and cyano; diazaspiro[4.4]nonanyl, substituted with 1 ═O substituent; oxa-azaspiro[4.4]nonanyl; oxa-azaspiro[3.5]nonanyl; oxa-azaspiro[4.5]decanyl, unsubstituted or substituted with 1 ═O substituent; oxadiazaspiro[3.4]octanyl, substituted with 1 ═O substituent; azaspiro[2.3]hexanyl; or oxa-azaspiro[bicyclo[3.1.0]hexane-pyrrolidinyl],
(12) azabicyclo[2.1.1]hexanyl, unsubstituted or substituted with 1-3 substituents independently selected from methyl, —CH₂OH, —OH, —C(CH₃)₂OH, and —OCH₃; azabicyclo[3.1.0]hexanyl, unsubstituted or substituted with 1 —C(O)OCH₂CH₃ substituent; oxa-azabicyclo[2.2.1]heptanyl; azabicyclo[3.1.1]heptanyl; azabicyclo[2.2.1]heptanyl, unsubstituted or substituted with 1-4 substituents independently selected from —OH, hydrogen, and methyl; oxaazabicyclo[3.2.0]heptanyl; or azabicyclo[3.2.0]heptanyl, substituted with 1-2 substituents independently selected from hydrogen, —OCH₂CF₃, —OCH₃ and —OH, or
(13) hexahydrocyclopenta[c]pyrrolyl, unsubstituted or substituted with 2 —F substituents; hexahydrofuro[3,2-b]pyrrolyl; octahydro-indolyl; hexahydropyrano[3,2-c]pyridinyl; hexahydrofuro[3,2-b]pyridinyl; or octahydrobenzo[b][1,4]oxazinyl, substituted with 1 methyl substituent.

9. The compound of claim 8, or a pharmaceutically acceptable salt thereof, wherein R² is

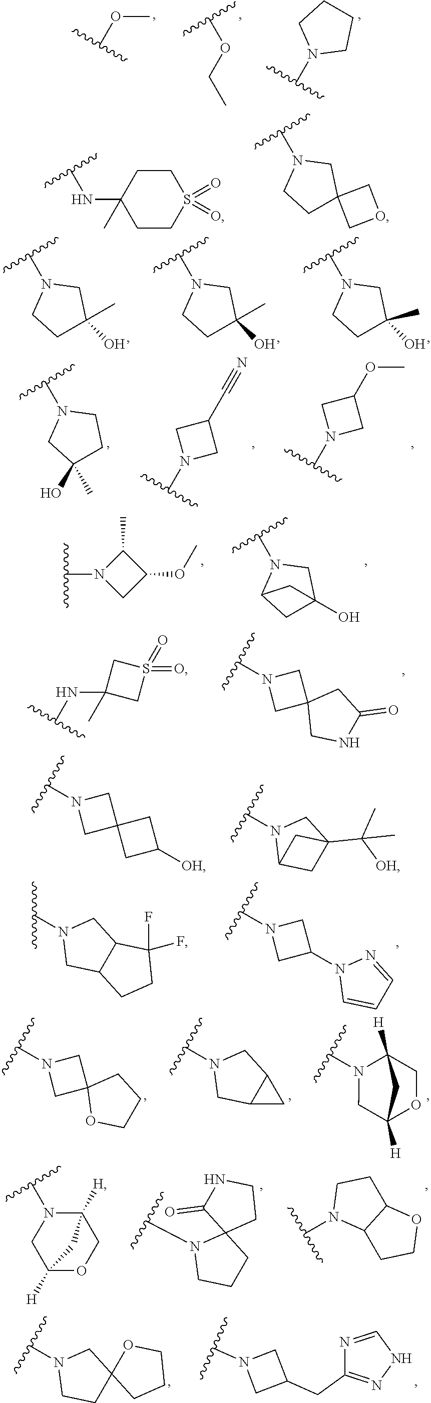

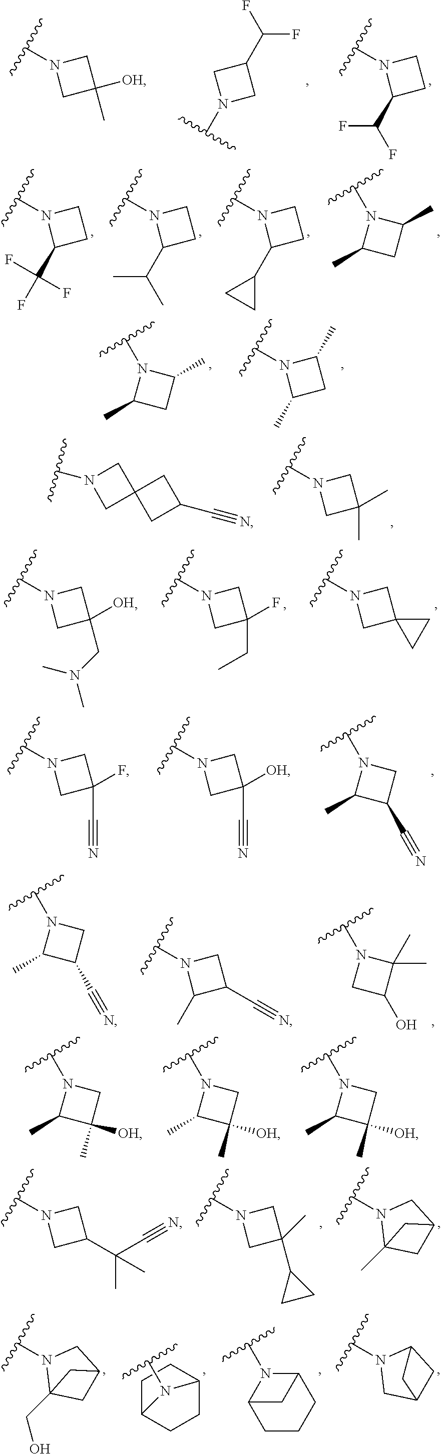

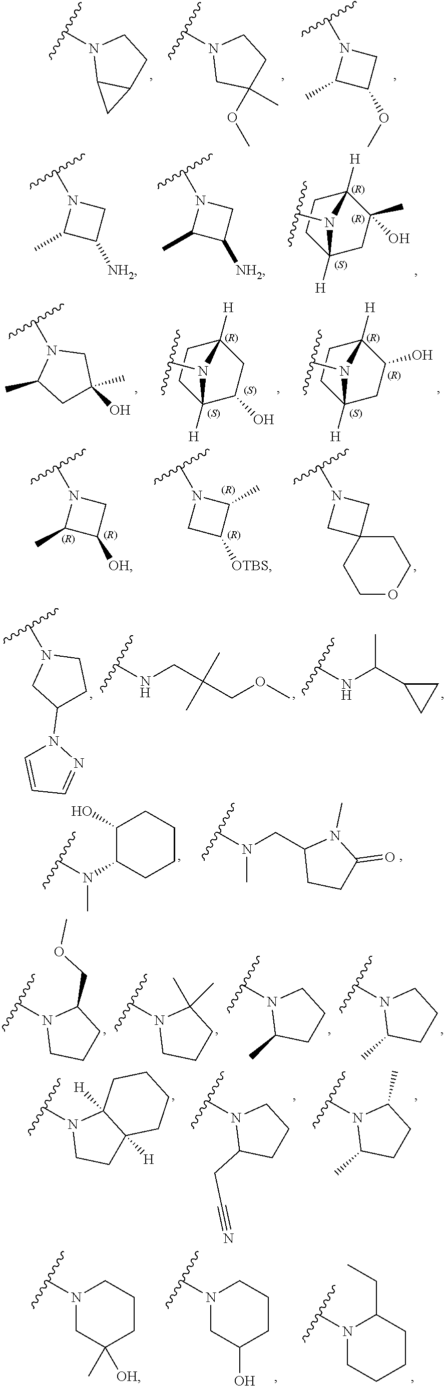

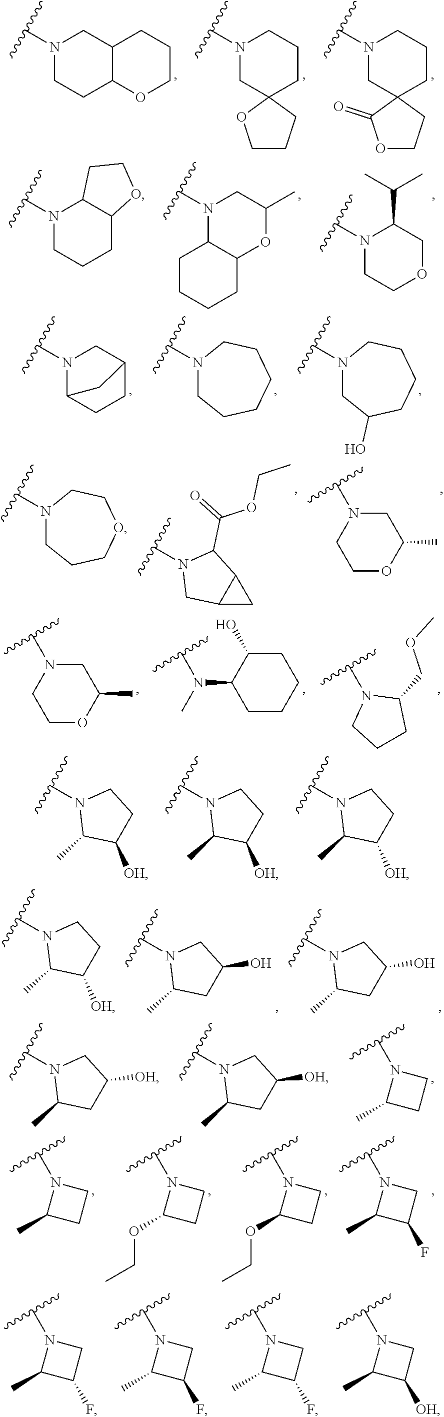

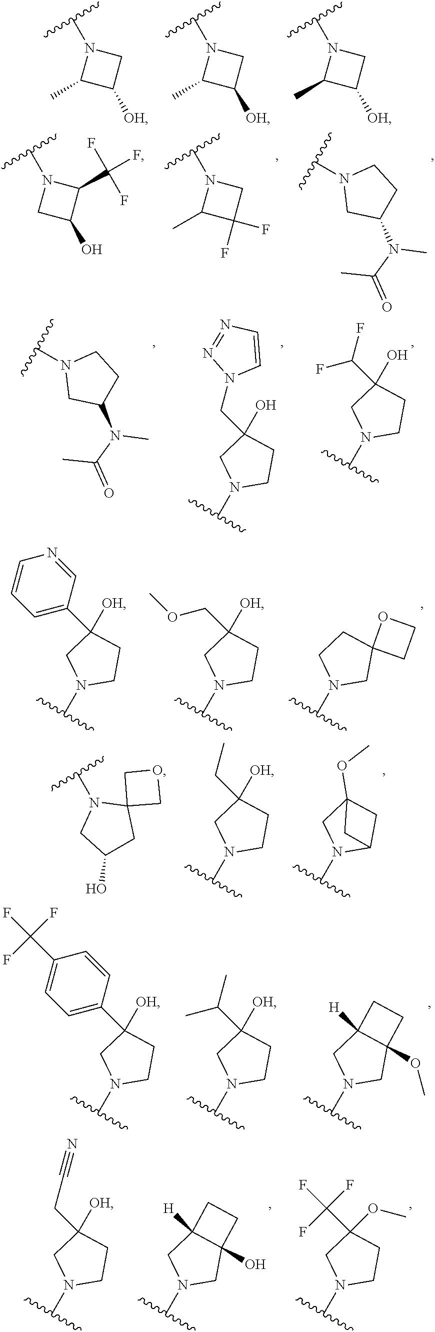

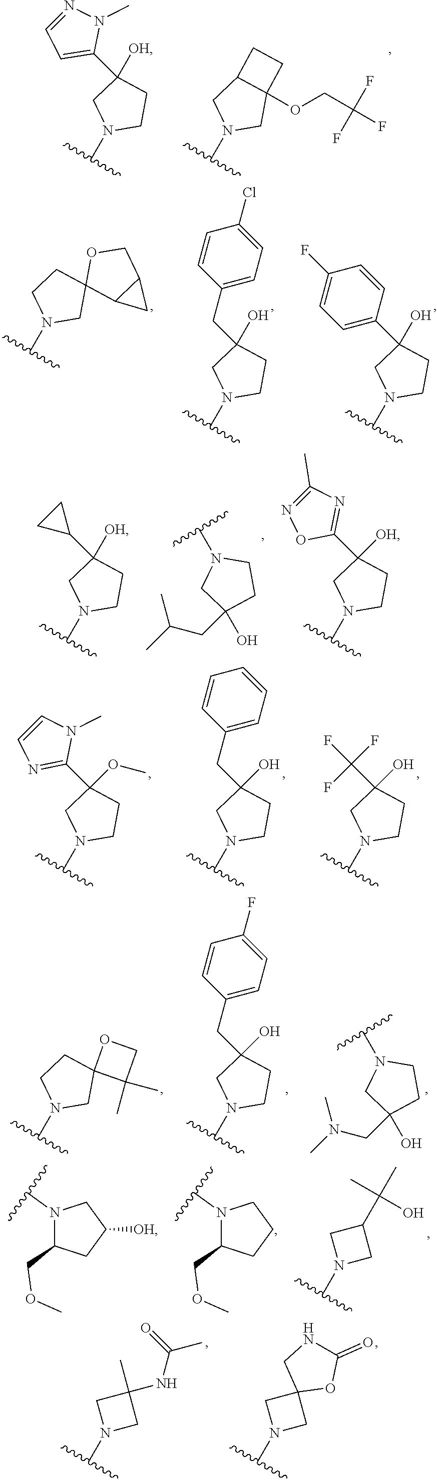

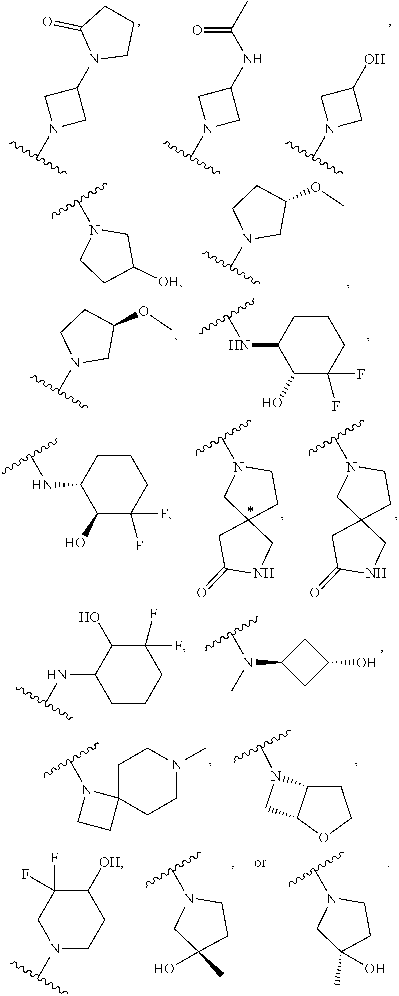

10. The compound of claim 1, or a pharmaceutically acceptable salt thereof, wherein R³ is hydrogen, —C₁₋₆alkyl, —C₃₋₆cycloalkyl, —C₁₋₆haloalkyl, —C₁₋₆alkyl-O—C₁₋₆alkyl, or —C₁₋₆alkyl-OH.

11. The compound of claim 10, or a pharmaceutically acceptable salt thereof, wherein R³ is hydrogen or —C₁₋₃alkyl.

12. The compound of claim 11, or a pharmaceutically acceptable salt thereof, wherein R³ is hydrogen or —CH₃.

13. The compound of claim 1, or a pharmaceutically acceptable salt thereof, wherein R⁴ is selected from hydrogen, halogen, —C₁₋₆alkyl, —C(O)NH—C₀₋₆alkyl, —C₀₋₆alkyl-(O)OC₁₋₆alkyl, —NH₂, —C₁₋₆haloalkyl, and —C₃₋₆cycloalkyl.

14. The compound of claim 13, or a pharmaceutically acceptable salt thereof, wherein R⁴ is selected from hydrogen, methyl, —C(O)NH—CH₃, —C(O)OCH₃, and —CH₂C(O)OCH₂CH₃.

15. The compound of claim 1, or a pharmaceutically acceptable salt thereof, wherein R⁵ is selected from hydrogen, halogen, —C₁₋₆alkyl, and C₁₋₆alkyl-(O)OC₁₋₆alkyl.

16. The compound of claim 15, or a pharmaceutically acceptable salt thereof, wherein R⁵ is hydrogen.

17. The compound of claim 1, or a pharmaceutically acceptable salt thereof, wherein R⁶ is selected from hydrogen, halogen, and —C₁₋₆alkyl.

18. The compound of claim 17, or a pharmaceutically acceptable salt thereof, wherein R⁶ is selected from hydrogen, —F, and —CH₃.

19. The compound of claim 1, or a pharmaceutically acceptable salt thereof, wherein X is selected from —O—, —NH—, —N—C₁₋₆alkyl-, and —S—.

20. The compound of claim 19, or a pharmaceutically acceptable salt thereof, wherein X is selected from —O— and —NH—.

21. The compound of claim 1, or a pharmaceutically acceptable salt thereof, wherein Y is selected from —O—, —S—, —N—, —C—R¹—, —C(H)—R¹—, —NR¹—, and —CH₂—.

22. The compound of claim 1, or a pharmaceutically acceptable salt thereof, wherein Z is selected from —C—R¹, —C(H)—R¹, —NH—, —S—, and —O—.

23. A compound of formula Ia: 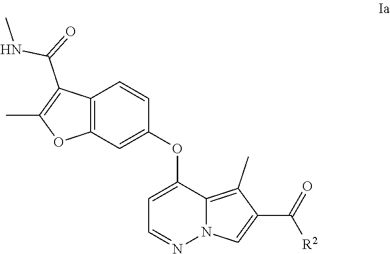
or a pharmaceutically acceptable salt thereof, wherein R² is selected from
(1) —O—C₁₋₆alkyl, and
(2) 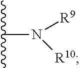
each of R⁹ and R¹⁰ is independently selected from:
(1) hydrogen,
(2) —C₀₋₆alkyl-(3-6 membered heterocyclyl), wherein the heterocyclyl has 1-3 ring heteroatoms selected from N, O, and S, and is unsubstituted or substituted with 1-3 substituents independently selected from R^{b},
(3) —C₁₋₆alkyl, unsubstituted or substituted with 1-3 substituents independently selected from R^{b},
(4) —C₁₋₆ alkyl-O—C₁₋₆alkyl, unsubstituted or substituted with 1-3 substituents independently selected from R^{b},
(5) —C₀₋₆alkyl-C₃₋₆cycloalkyl, wherein the cycloalkyl is unsubstituted or substituted with 1-3 substituents independently selected from R^{b}, and
(6) 5-10 membered bicyclic or tricyclic cycloalkyl, wherein the cycloalkyl is unsubstituted or substituted with 1-3 substituents independently selected from R^{b};
when present, R^{b} is selected from
(1) —C₁₋₆alkyl,
(2) halo,
(3) —NH₂,
(4) ═O, and
(5) —OH;
or alternatively, R⁹ and R¹⁰ together with the nitrogen to which they are attached form a 3-10 membered heterocyclyl having 0-3 ring heteroatoms independently selected from N, O, and S, in addition to the nitrogen to which R⁹ and R¹⁰ are attached, wherein the heterocyclyl is optionally substituted with 1-4 substituents independently selected from R^{c};
when present, R^{c} is selected from
(1) —C₀₋₆alkyl-OH,
(2) —C₁₋₆alkyl, unsubstituted or substituted with 1-3 substituents independently selected from —OH,
(3) —C₀₋₆alkyl-cyano,
(4) —C₀₋₆alkyl-O—C₁₋₃alkyl,
(5) —C₀₋₃alkyl-(5-6 membered monocyclic heteroaryl), wherein the heteroaryl has 1-3 ring heteroatoms selected from N, O, and S, and is unsubstituted or substituted with 1-3 substituents independently selected from C₁₋₆alkyl,
(6) halo,
(7) —C₁₋₆haloalkyl,
(8) C₀₋₆alkyl-aryl, wherein the aryl is unsubstituted or substituted with 1-3 substituents independently selected from C₁₋₆haloalkyl and halo,
(9) —C₃₋₆cycloalkyl,
(10) —C₀₋₆alkyl-N, unsubstituted or substituted with 1-3 substituents independently selected from hydrogen and C₁₋₆alkyl,
(11) —NH—C(O)C₁₋₆alkyl,
(12) —C₀₋₃alkyl-(3-6 membered monocyclic heterocyclyl), wherein the heterocyclyl has 1-3 ring heteroatoms selected from N, O, and S, and is unsubstituted or substituted with 1 ═O substituent;
(13) —O—C₁₋₆haloalkyl,
(14) ═O,
(15) hydrogen, and
(16) —C(O)OC₁₋₆alkyl.

24. The compound of claim 23, or a pharmaceutically acceptable salt thereof, wherein R² is
(1) —OC₁₋₂alkyl,
(2) —NH—C₀₋₃alkyl-3-6 membered heterocyclyl, wherein the heterocyclyl has 1 ring heteroatom selected from S and O, and is unsubstituted or substituted with 1-3 substituents independently selected from —C₁₋₆alkyl, halo, ═O, and —OH,
(3) —NH—C₁₋₅alkyl, unsubstituted or substituted with 1-3 substituents independently selected from —C₁₋₆alkyl, halo, ═O, and —OH,
(4) —NH—C₀₋₃alkyl-C₃₋₆cycloalkyl, unsubstituted or substituted with 1-3 substituents independently selected from —C₁₋₆alkyl, halo, ═O, and —OH,
(5) 3-7 membered monocyclic heterocyclyl having 0-3 ring heteroatoms independently selected from N, O, and S, in addition to the nitrogen to which R⁹ and R¹⁰ are attached, wherein the monocyclic heterocyclyl is unsubstituted or substituted with 1-4 substituents independently selected from: —C₁₋₆alkyl, unsubstituted or substituted with 1-3 substituents selected from —OH; —NH₂; —C₀₋₆alkyl-cyano; —C₀₋₆alkyl-O—C₁₋₃alkyl; —C₀₋₃alkyl-5-6 membered monocyclic heteroaryl having 1-3 ring heteroatoms selected from N, O, and S, unsubstituted or substituted with 1-3 substituents selected from —C₁₋₆alkyl; halo; —OH; —C₁₋₆haloalkyl; C₀₋₆alkyl-aryl, wherein the aryl is unsubstituted or substituted with 1-3 substituents independently selected from —C₁₋₆haloalkyl, and halo; —C₃₋₆cycloalkyl; —C₀₋₆alkyl-N, unsubstituted or substituted with 1-3 substituents independently selected from hydrogen and —C₁₋₆alkyl; —NH—C(O)C₁₋₆alkyl; —N(CH₃)—C(O)C₁₋₆alkyl; and —C₀₋₃alkyl-3-6 membered monocyclic heterocyclyl having 1-3 ring heteroatoms independently selected from N, O, and S, unsubstituted or substituted with 1-3 ═O substituents,
(6) 5-10 membered spiro bicyclic heterocyclyl having 0-3 ring heteroatoms independently selected from N, O, and S, in addition to the nitrogen to which R⁹ and R¹⁰ are attached, wherein the spiro bicyclic heterocyclyl is unsubstituted or substituted with 1-3 substituents independently selected from cyano, ═O, —OH, and —C₁₋₆alkyl,
(7) 5-10 membered bridged heterocyclyl having 0-3 ring heteroatoms independently selected from N, O, and S, in addition to the nitrogen to which R⁹ and R¹⁰ are attached, wherein the bridged heterocyclyl is unsubstituted or substituted with 1-3 substituents independently selected from methyl, —C₀₋₆alkyl-OH, and hydrogen, or
(8) 5-10 membered fused bicyclic heterocyclyl having 0-3 ring heteroatoms independently selected from N, O, and S, in addition to the nitrogen to which R⁹ and R¹⁰ are attached, wherein the fused bicyclic heterocyclyl is unsubstituted or substituted with 1-3 substituents independently selected from halo, hydrogen, —C(O)OC₁₋₆alkyl, —O—C₁₋₆alkyl, —OH, and —O—C₁₋₆haloalkyl.

25. The compound of claim 24, or a pharmaceutically acceptable salt thereof, wherein R² is
(1) —OCH₂CH₃,
(2) —NH-thietanyl, substituted with 2 ═O substituents and 1 methyl substituent; —NH-tetrahydrothiopyranyl, substituted with 2 ═O substituents and 1 methyl substituent; or —N(CH₃)CH₂-pyrrolidinyl, wherein the pyrrolidinyl is substituted 2 substituents independently selected from methyl and ═O,
(3) —NH—CH₂C(CH₃)₂CH₂OCH₃,
(4) —NH-cyclohexnanyl, substituted with 1-3 substituents independently selected from —OH and —F; —N(CH₃)-cyclohexanyl substituted with 1 —OH substituent; or —NHCH(CH₃)-cyclopropanyl,
(5) pyrrolidinyl, unsubstituted or substituted with 1-3 substituents independently selected from: —OH; pyrazolyl, unsubstituted or substituted with 1 methyl substituent; —CH₂OCH₃; —C₀₋₁alkyl-cyano; —N(CH₃)C(O)CH₃; —CF₃; —C₁₋₄alkyl; —C₀₋₁alkyl-phenyl, unsubstituted or substituted with 1 substituent selected from —C₁, —F, and —CF₃; —OCH₃; cyclopropyl; oxadiazolyl, substituted with 1 methyl substituent; and —CH₂N(CH₃)₂,
(6) azetidinyl, unsubstituted or substituted with 1-3 substituents independently selected from —C₀₋₃alkyl-cyano, —OCH₃, pyrazole, —CH₂-triazole, —OH, —C₁₋₂alkyl, —CH(CH₃)₂, —CH₂OCH₃, —F, —CHF₂, —CF₃, —NH₂, cyclopropyl, and —CH₂N(CH₃)₂,
(7) morpholinyl, substituted with 1 substituent selected from —CH(CH₃)₂ and methyl,
(8) azepanyl, unsubstituted or substituted with 1 —OH substituent,
(9) oxazepanyl,
(10) oxa-azaspiro[3.4]octanyl, unsubstituted or substituted with 2 methyl substituents; diazaspiro[3.4]octanyl, substituted with 1 ═O substituent and 1 hydrogen substituent; azaspiro[3.3]heptanyl, substituted with 1 substituent selected from —OH and cyano; diazaspiro[4.4]nonanyl, substituted with 1 ═O substituent; oxa-azaspiro[4.4]nonanyl; oxa-azaspiro[3.5]nonanyl; oxa-azaspiro[4.5]decanyl, unsubstituted or substituted with 1 ═O substituent; azaspiro[2.3]hexanyl; or oxa-azaspiro[bicyclo[3.1.0]hexane-pyrrolidinyl],
(11) azabicyclo[2.1.1]hexanyl, unsubstituted or substituted with 1-3 substituents selected from methyl, —CH₂OH, —OH, —C(CH₃)₂OH, —OCH₃; azabicyclo[3.1.0]hexanyl, unsubstituted or substituted with 1 —C(O)OCH₂CH₃ substituent; oxa-azabicyclo[2.2.1]heptanyl; azabicyclo[3.1.1]heptanyl; azabicyclo[2.2.1]heptanyl, unsubstituted or substituted with 1-4 substituents independently selected from —OH, hydrogen, and methyl; or azabicyclo[3.2.0]heptanyl, substituted with 1-2 substituents independently selected from hydrogen, —OCH₂CF₃, —OCH₃, and —OH, or
(12) hexahydrocyclopenta[c]pyrrolyl, substituted with 2 —F substituents; hexahydrofuro[3,2-b]pyrrolyl; octahydro-indolyl; hexahydropyrano[3,2-c]pyridinyl; hexahydrofuro[3,2-b]pyridinyl; or octahydrobenzo[b][1,4]oxazinyl, substituted with 1 methyl substituent.

26. The compound of claim 25, or a pharmaceutically acceptable salt thereof, wherein R² is

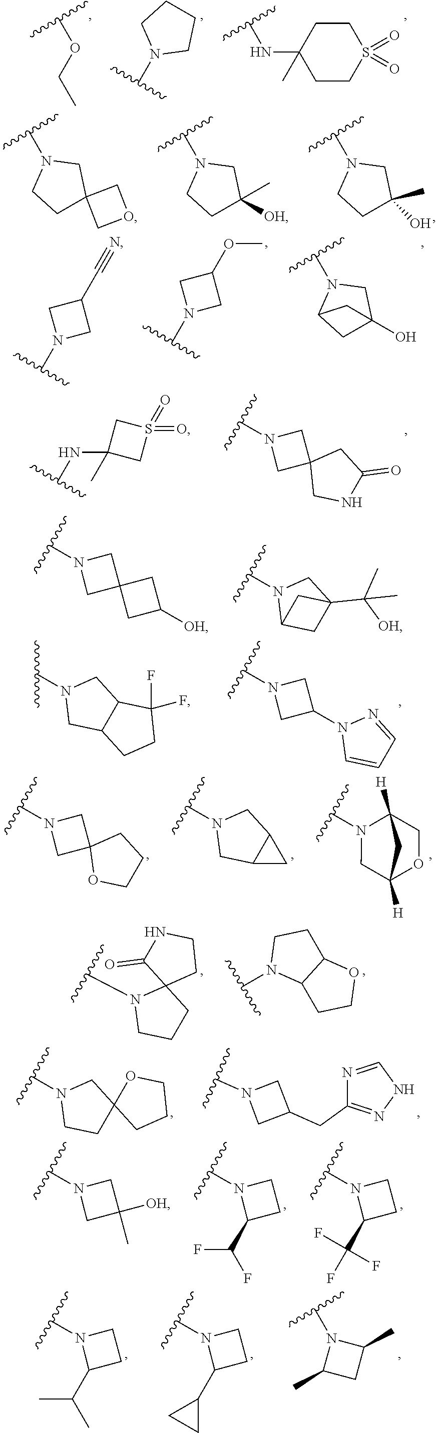

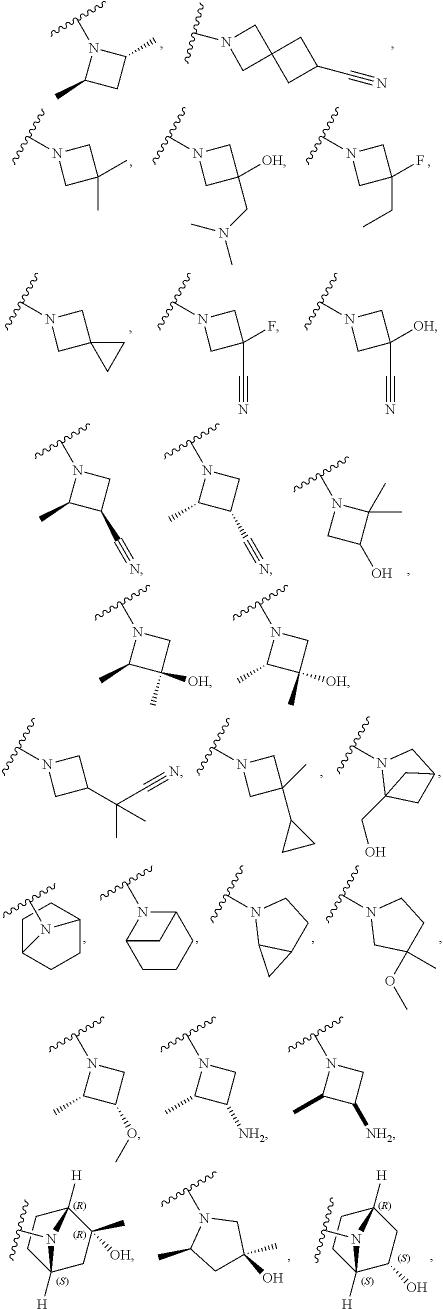

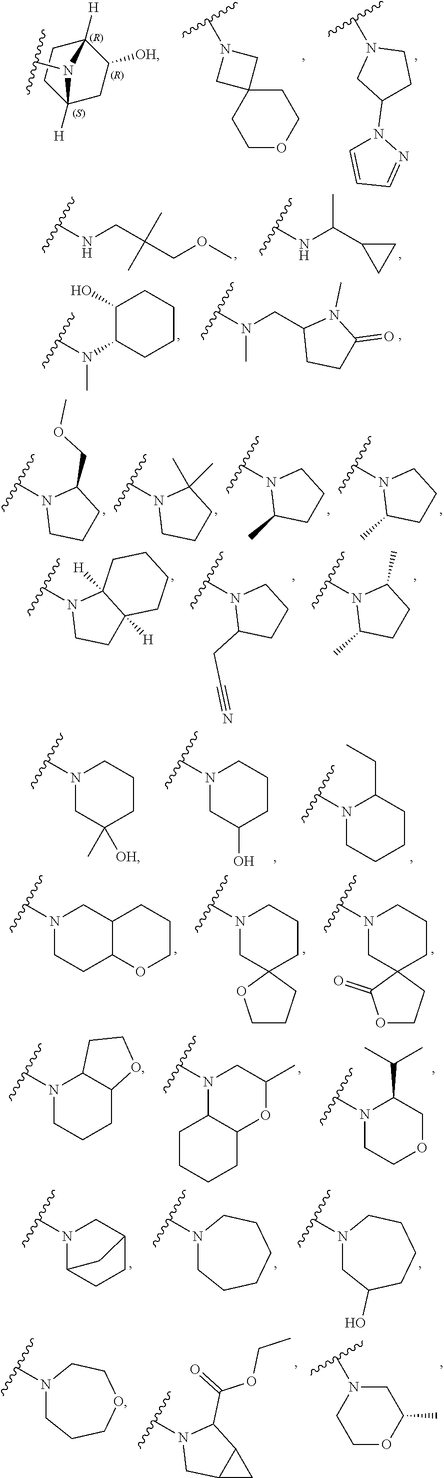

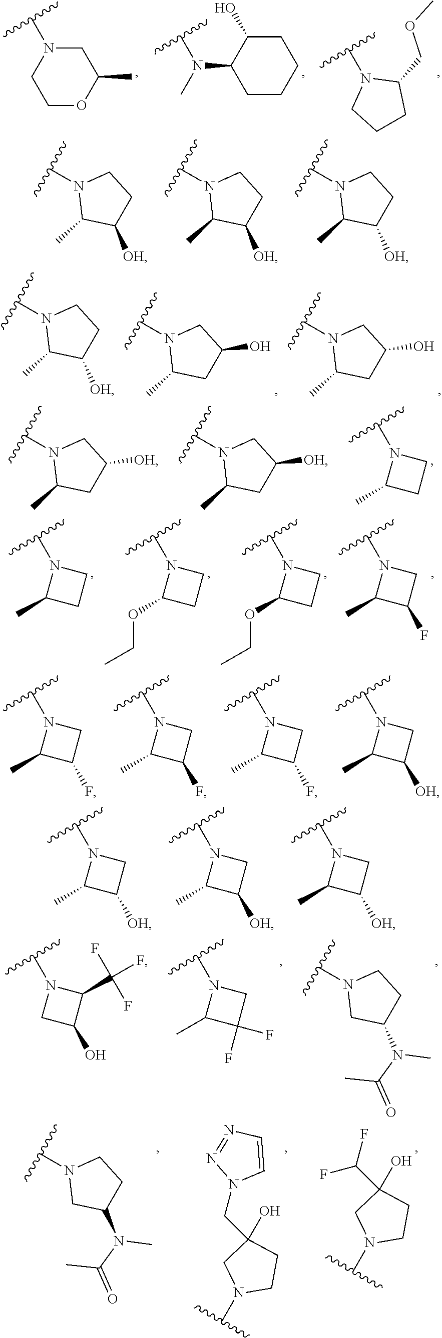

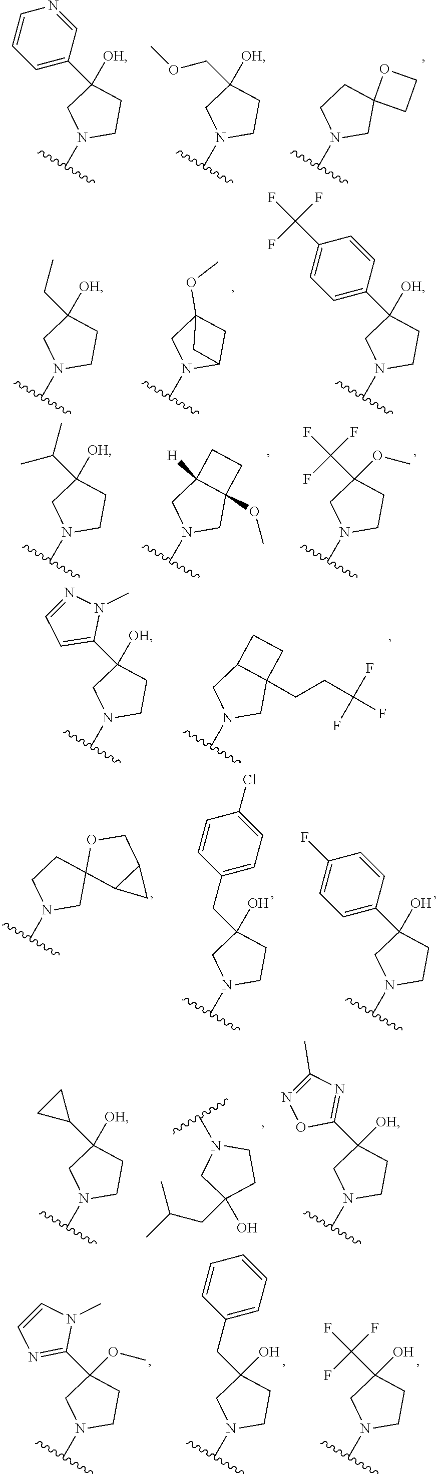

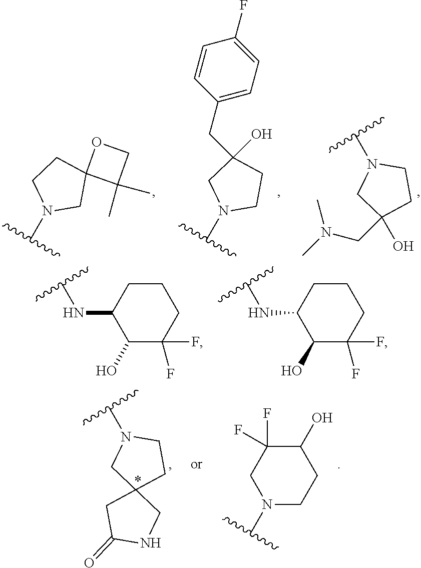

27. A compound or pharmaceutically acceptable salt thereof, selected from:

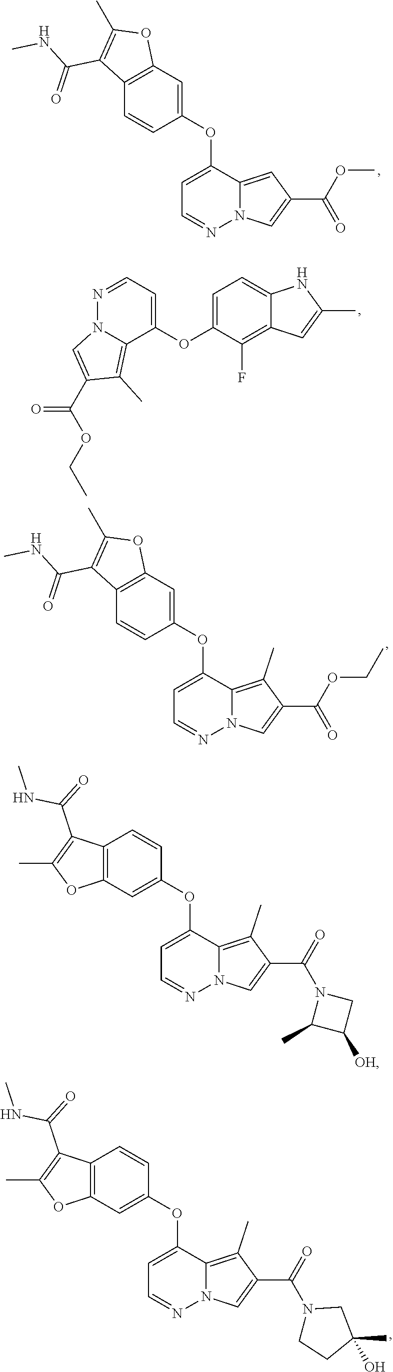

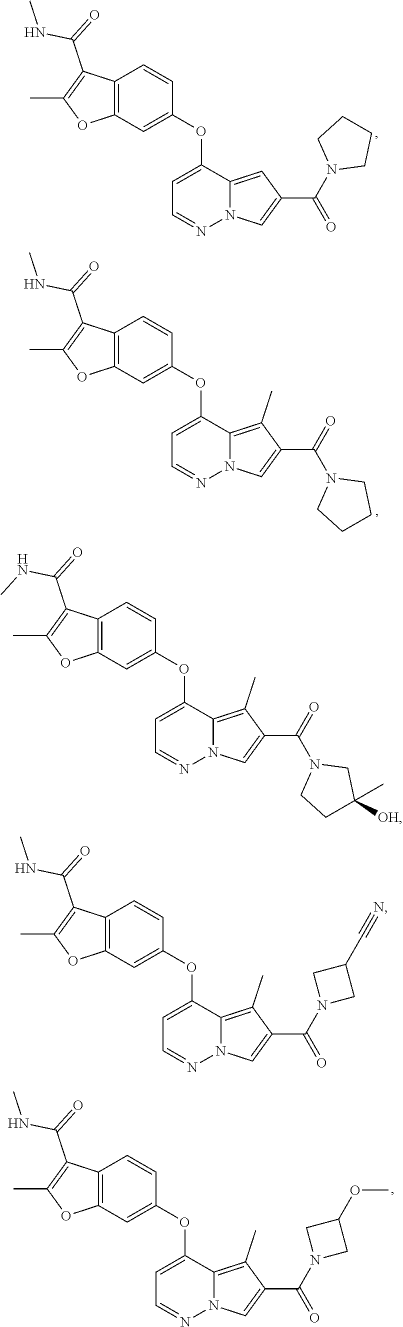

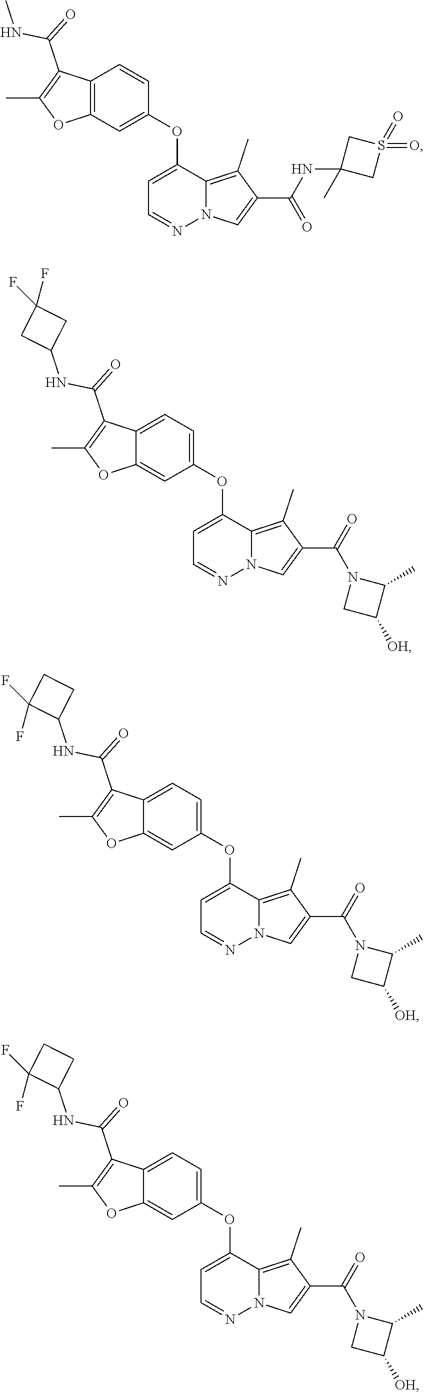 [SMILES: CNC(=O)c1c(C)oc2cc(Oc3ccnn4cc(C(=O)NC5(C)CS(=O)(=O)C5)c(C)c34)ccc12 ; Cc1oc2cc(Oc3ccnn4cc(C(=O)N5C[C@@H](O)[C@H]5C)c(C)c34)ccc2c1C(=O)NC1CC(F)(F)C1 ; Cc1oc2cc(Oc3ccnn4cc(C(=O)N5C[C@@H](O)[C@H]5C)c(C)c34)ccc2c1C(=O)NC1CCC1(F)F ; Cc1oc2cc(Oc3ccnn4cc(C(=O)N5C[C@@H](O)[C@H]5C)c(C)c34)ccc2c1C(=O)NC1CCC1(F)F]

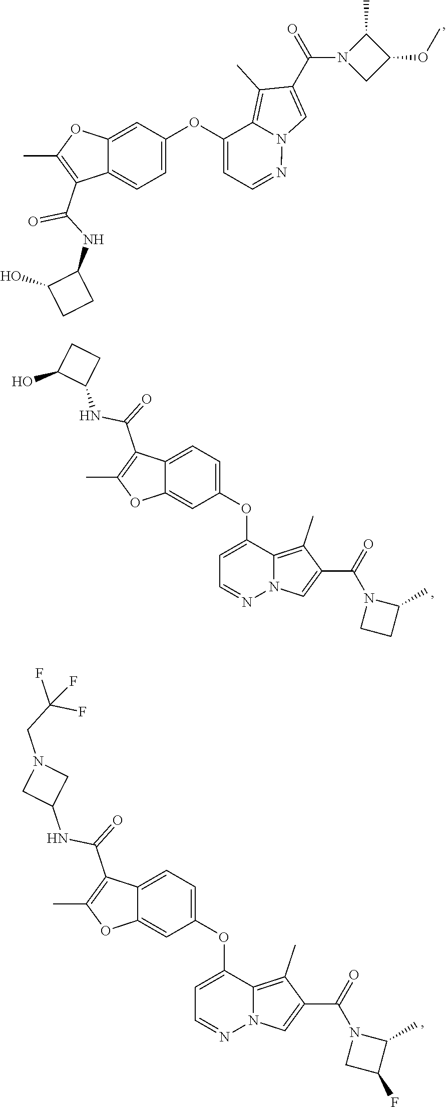

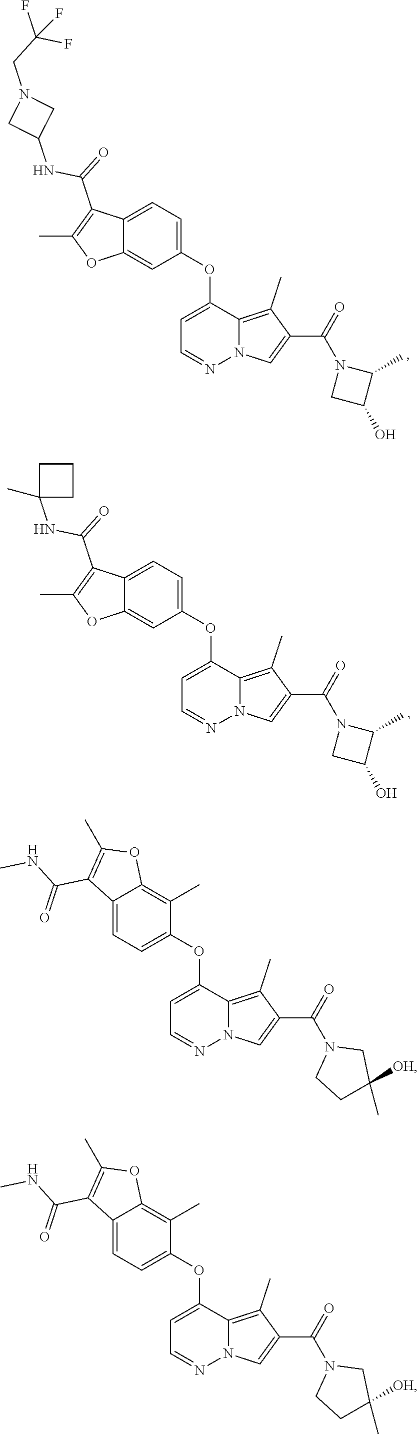

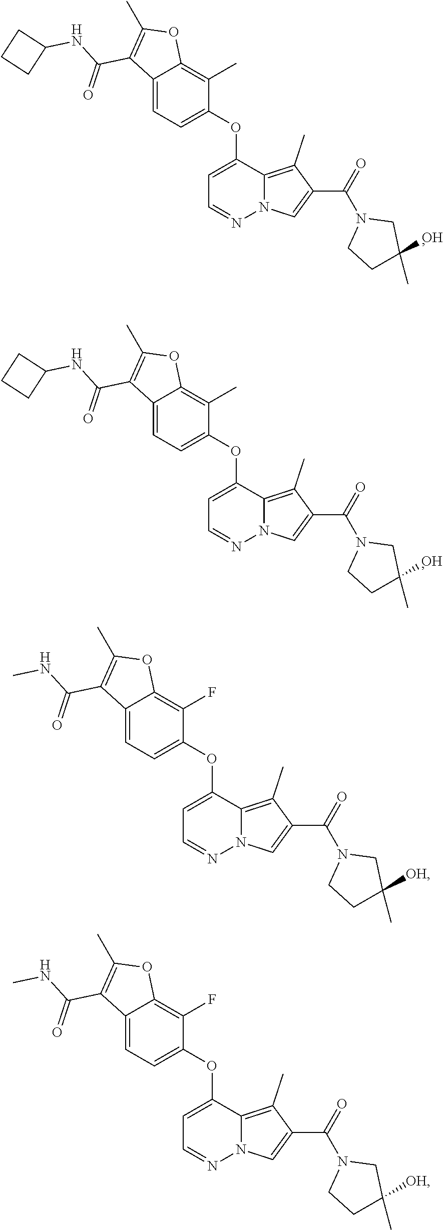 [SMILES: Cc1oc2c(C)c(Oc3ccnn4cc(C(=O)N5CC[C@](C)(O)C5)c(C)c34)ccc2c1C(=O)NC1CCC1 ; Cc1oc2c(C)c(Oc3ccnn4cc(C(=O)N5CC[C@@](C)(O)C5)c(C)c34)ccc2c1C(=O)NC1CCC1 ; CNC(=O)c1c(C)oc2c(F)c(Oc3ccnn4cc(C(=O)N5CC[C@](C)(O)C5)c(C)c34)ccc12 ; CNC(=O)c1c(C)oc2c(F)c(Oc3ccnn4cc(C(=O)N5CC[C@@](C)(O)C5)c(C)c34)ccc12]

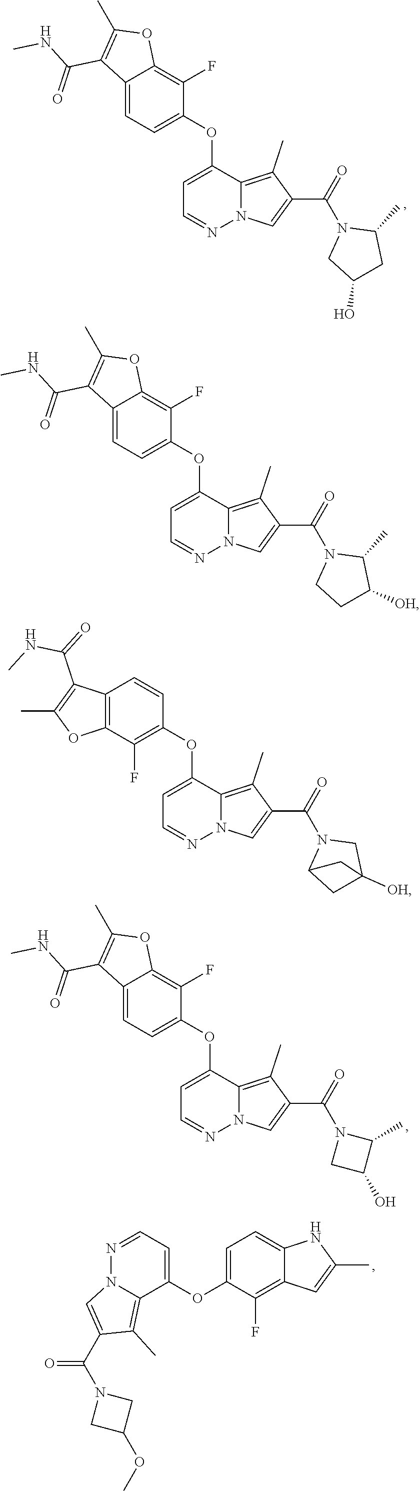

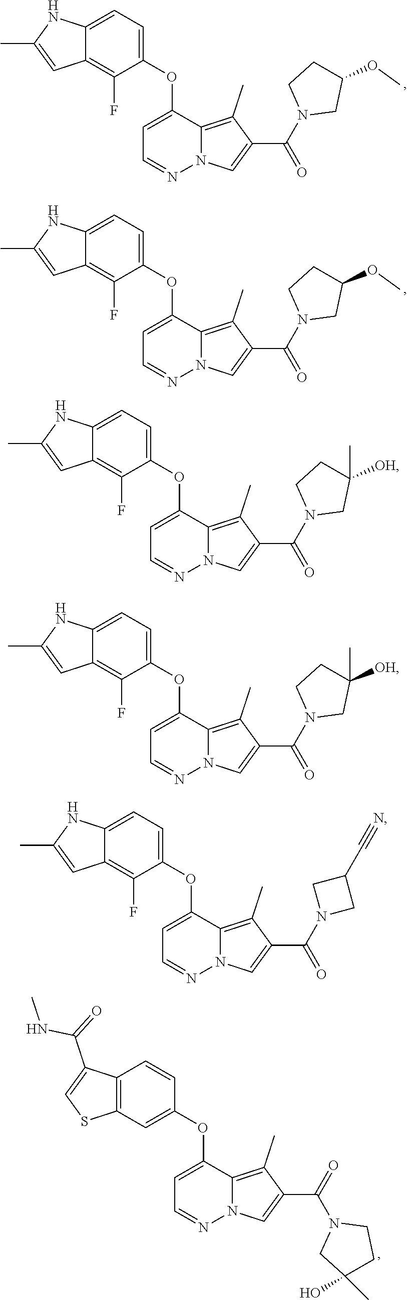

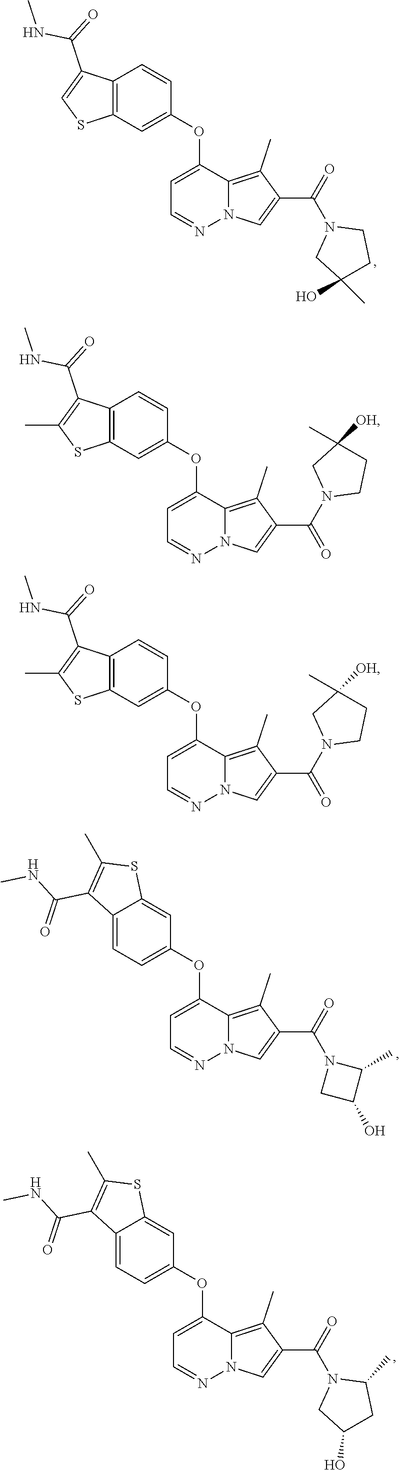

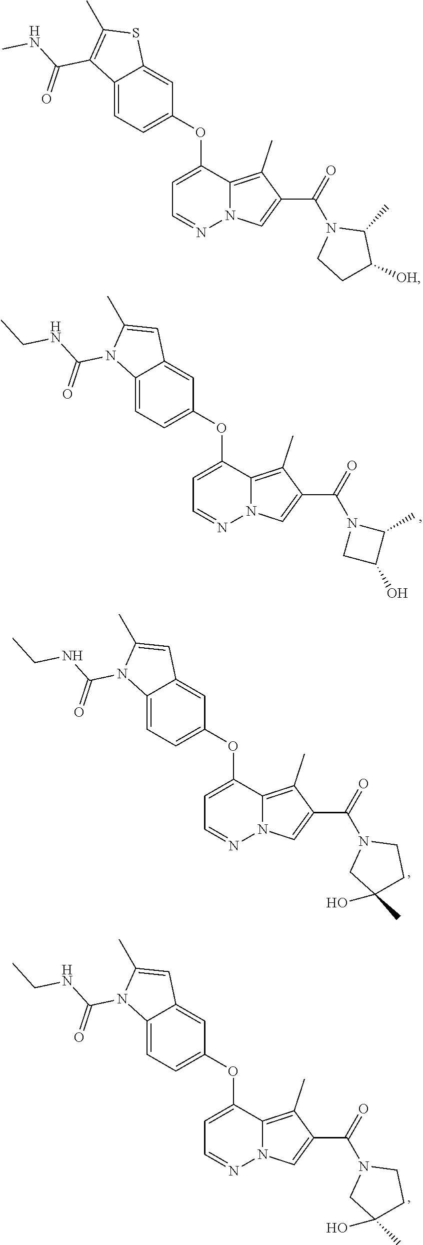

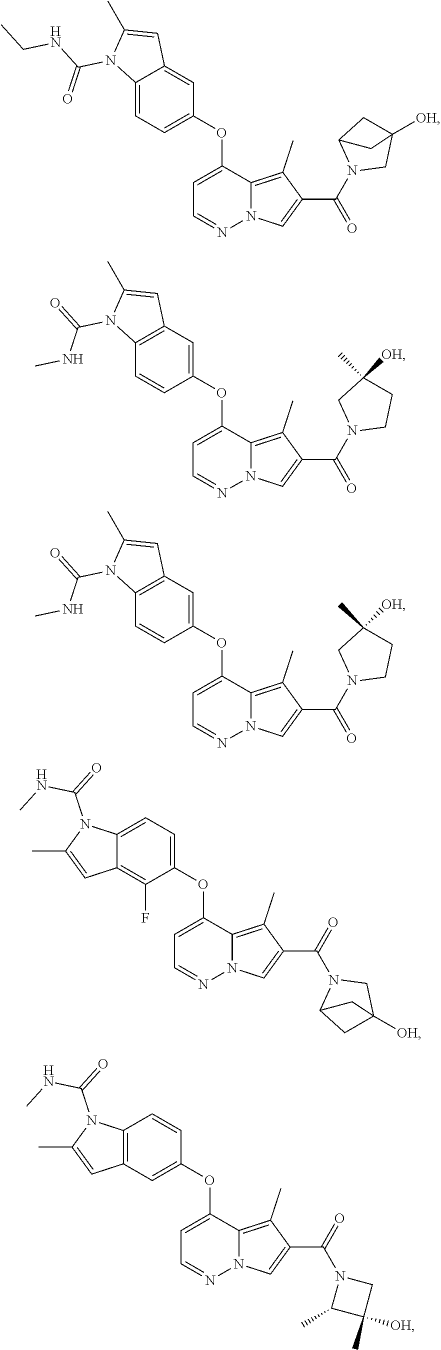 [SMILES: CCNC(=O)n1c(C)cc2cc(Oc3ccnn4cc(C(=O)N5CC6(O)CC5C6)c(C)c34)ccc21 ; CNC(=O)n1c(C)cc2cc(Oc3ccnn4cc(C(=O)N5CC[C@](C)(O)C5)c(C)c34)ccc21 ; CNC(=O)n1c(C)cc2cc(Oc3ccnn4cc(C(=O)N5CC[C@@](C)(O)C5)c(C)c34)ccc21 ; CNC(=O)n1c(C)cc2c(F)c(Oc3ccnn4cc(C(=O)N5CC6(O)CC5C6)c(C)c34)ccc21 ; CNC(=O)n1c(C)cc2cc(Oc3ccnn4cc(C(=O)N5C[C@](C)(O)[C@@H]5C)c(C)c34)ccc21]

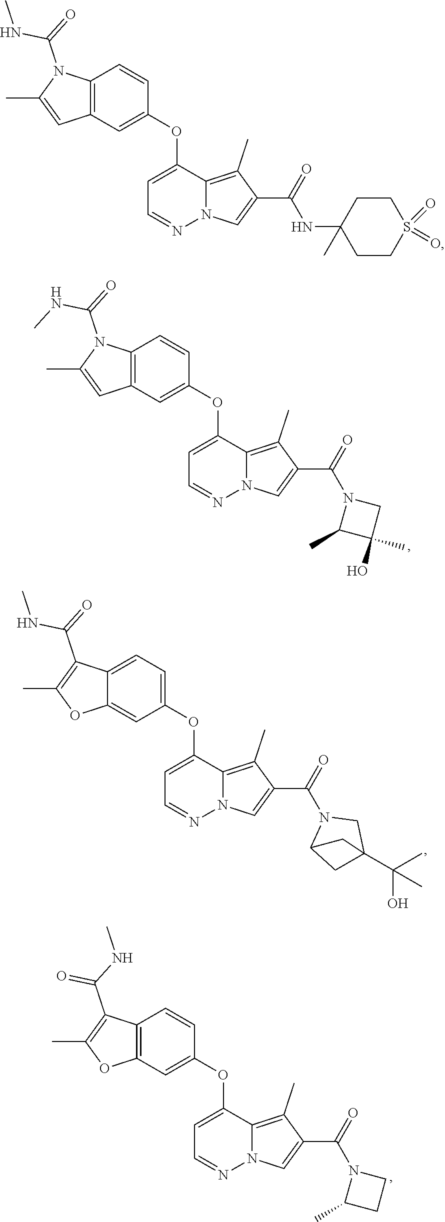

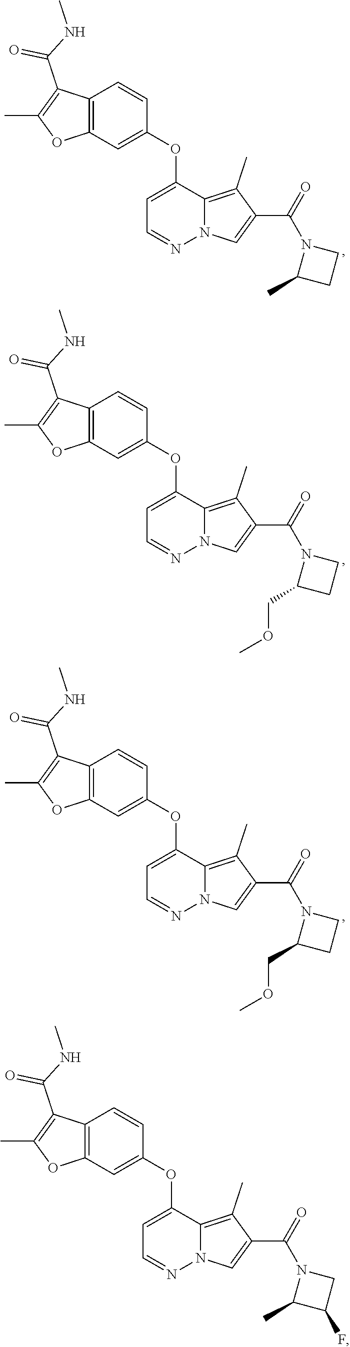 [SMILES: CNC(=O)c1c(C)oc2cc(Oc3ccnn4cc(C(=O)N5CC[C@H]5C)c(C)c34)ccc12 ; CNC(=O)c1c(C)oc2cc(Oc3ccnn4cc(C(=O)N5CC[C@@H]5COC)c(C)c34)ccc12 ; CNC(=O)c1c(C)oc2cc(Oc3ccnn4cc(C(=O)N5CC[C@H]5COC)c(C)c34)ccc12 ; CNC(=O)c1c(C)oc2cc(Oc3ccnn4cc(C(=O)N5C[C@@H](F)[C@H]5C)c(C)c34)ccc12]

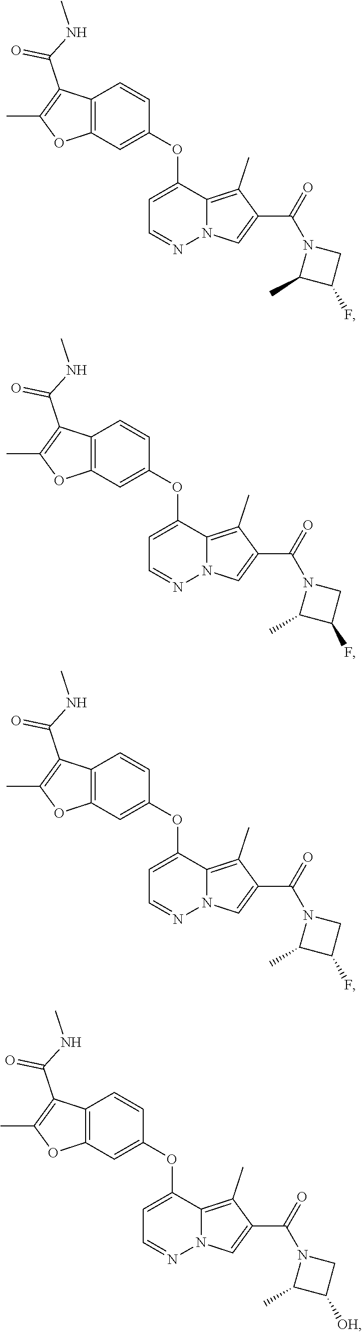 [SMILES: CNC(=O)c1c(C)oc2cc(Oc3ccnn4cc(C(=O)N5C[C@H](F)[C@H]5C)c(C)c34)ccc12 ; CNC(=O)c1c(C)oc2cc(Oc3ccnn4cc(C(=O)N5C[C@@H](F)[C@@H]5C)c(C)c34)ccc12 ; CNC(=O)c1c(C)oc2cc(Oc3ccnn4cc(C(=O)N5C[C@H](F)[C@@H]5C)c(C)c34)ccc12 ; CNC(=O)c1c(C)oc2cc(Oc3ccnn4cc(C(=O)N5C[C@H](O)[C@@H]5C)c(C)c34)ccc12]

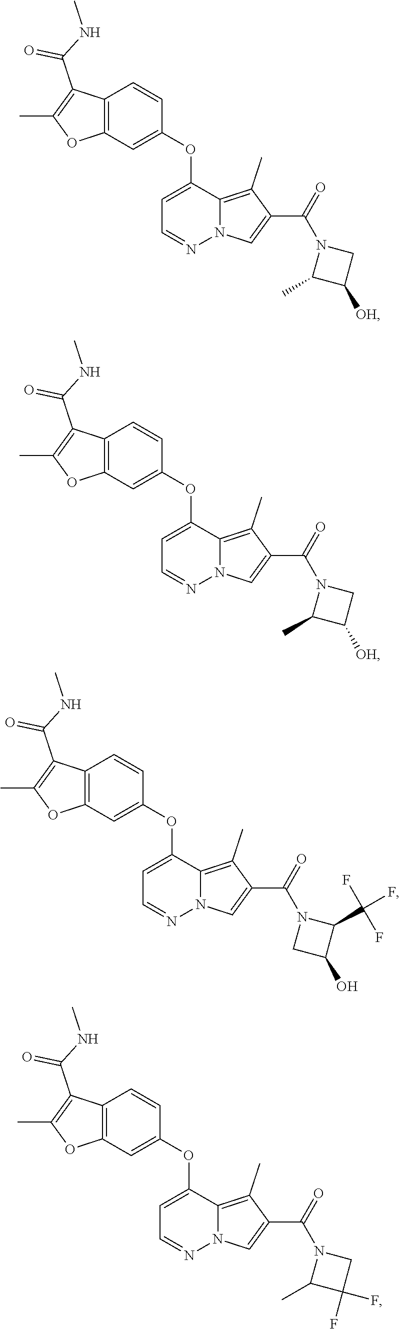 [SMILES: CNC(=O)c1c(C)oc2cc(Oc3ccnn4cc(C(=O)N5C[C@@H](O)[C@@H]5C)c(C)c34)ccc12 ; CNC(=O)c1c(C)oc2cc(Oc3ccnn4cc(C(=O)N5C[C@H](O)[C@H]5C)c(C)c34)ccc12 ; CNC(=O)c1c(C)oc2cc(Oc3ccnn4cc(C(=O)N5C[C@H](O)[C@@H]5C(F)(F)F)c(C)c34)ccc12 ; CNC(=O)c1c(C)oc2cc(Oc3ccnn4cc(C(=O)N5CC(F)(F)C5C)c(C)c34)ccc12]

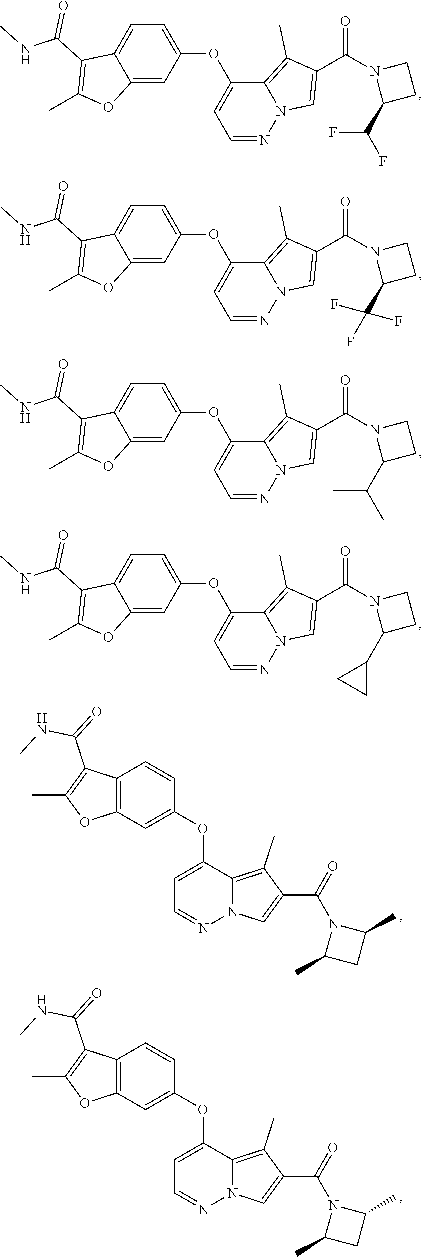

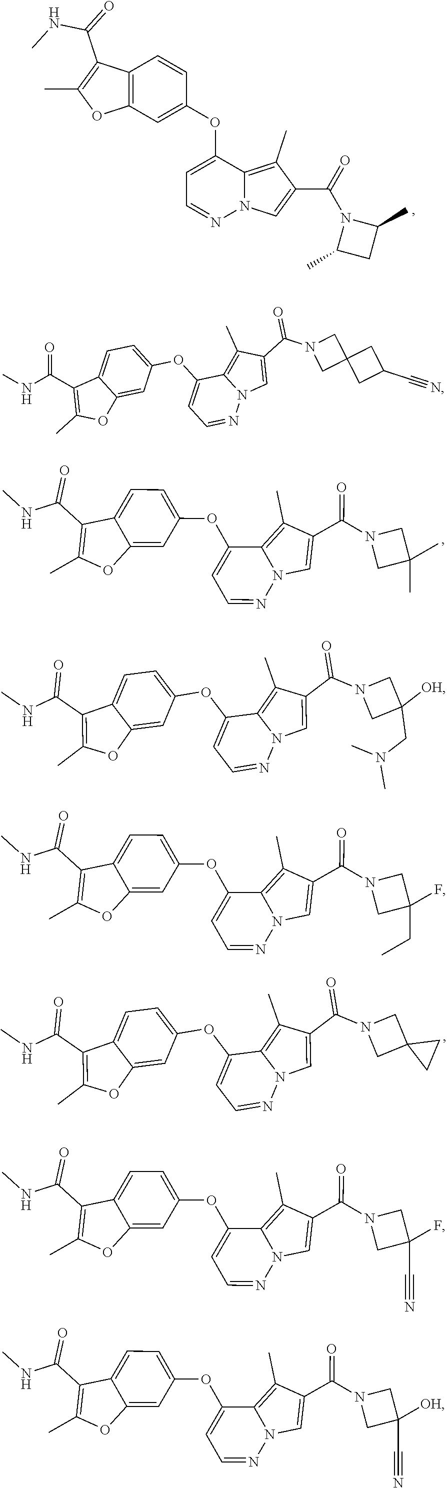

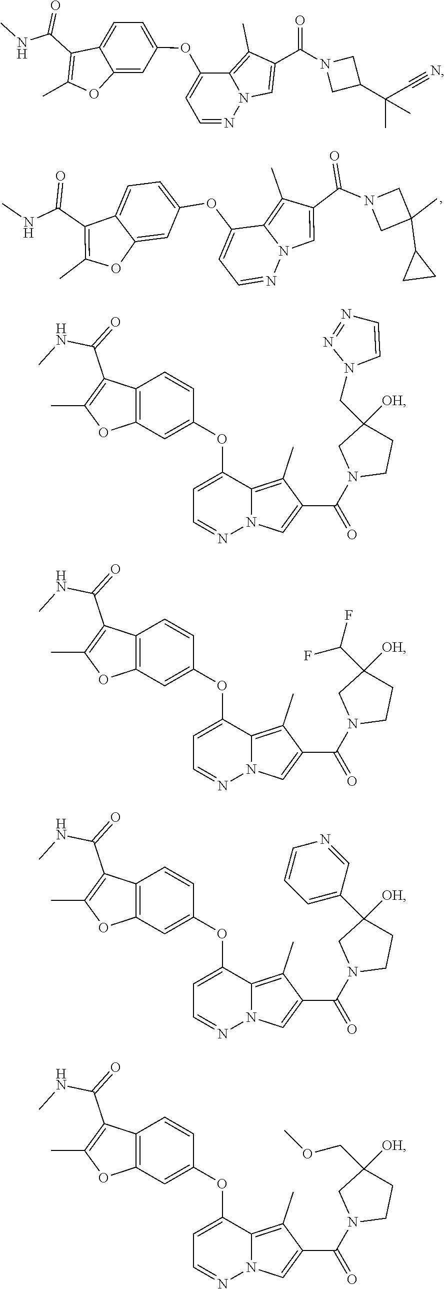

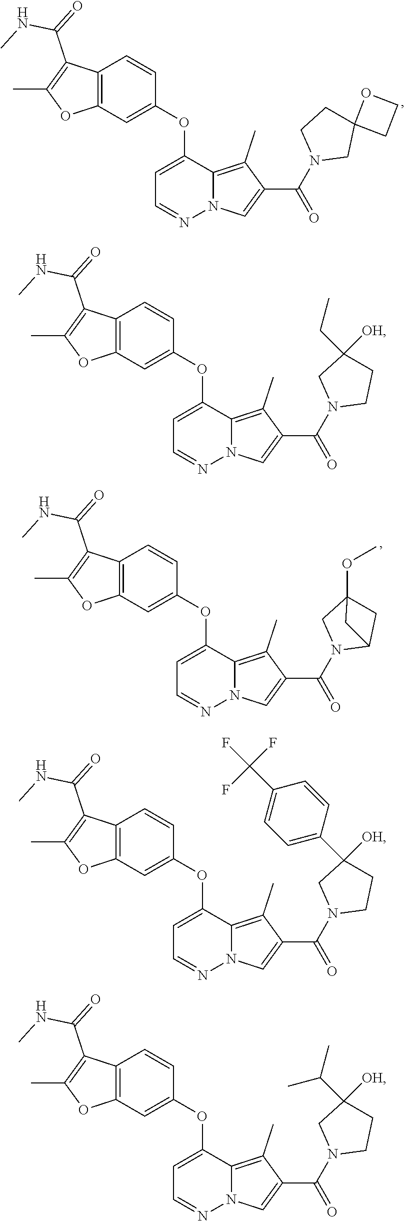

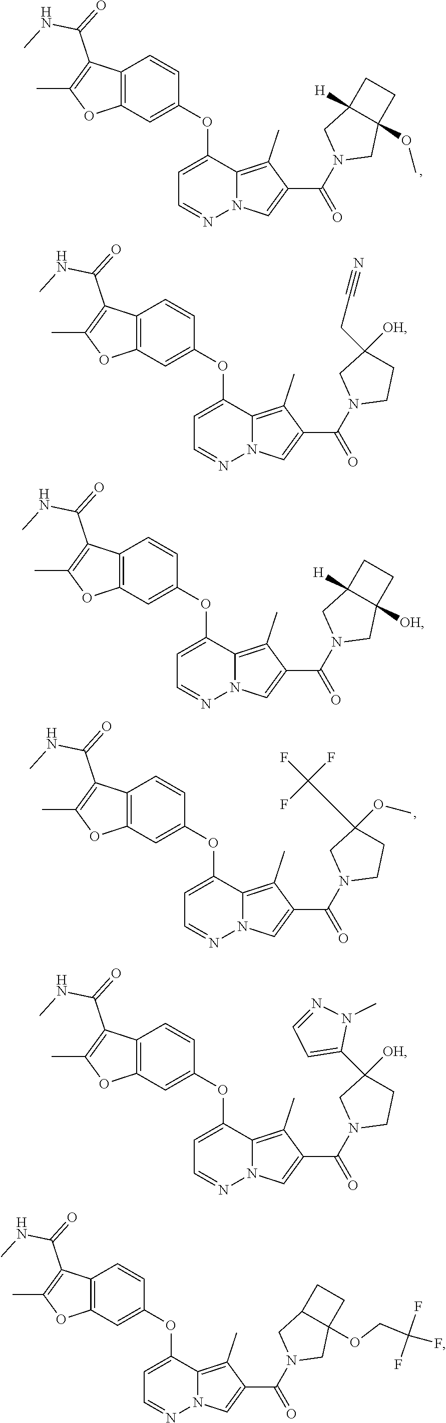

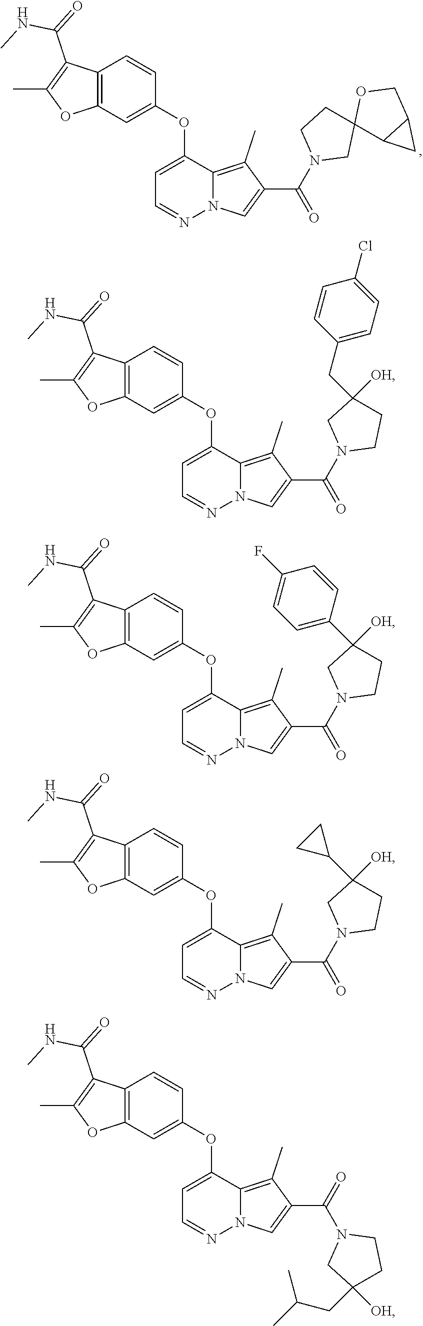

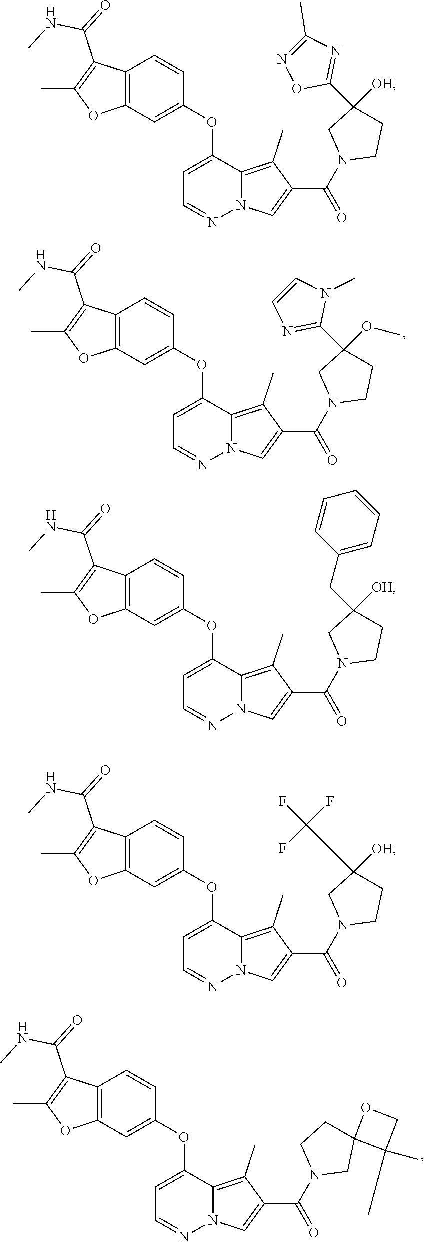

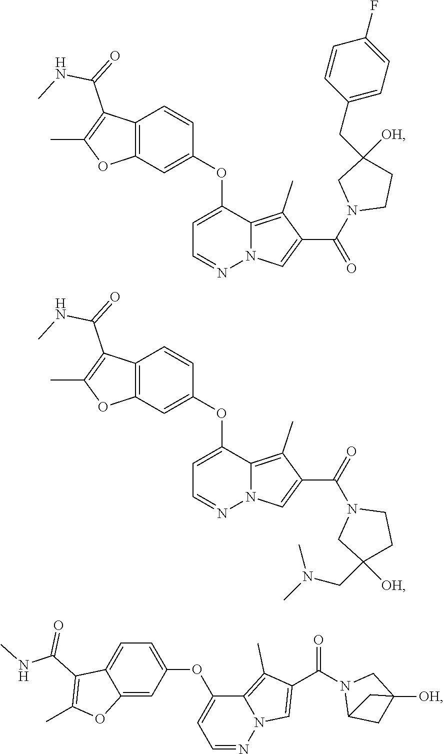 [SMILES: CNC(=O)c1c(C)oc2cc(Oc3ccnn4cc(C(=O)N5CCC(O)(Cc6ccc(F)cc6)C5)c(C)c34)ccc12 ; CNC(=O)c1c(C)oc2cc(Oc3ccnn4cc(C(=O)N5CCC(O)(CN(C)C)C5)c(C)c34)ccc12 ; CNC(=O)c1c(C)oc2cc(Oc3ccnn4cc(C(=O)N5CC6(O)CC5C6)c(C)c34)ccc12]

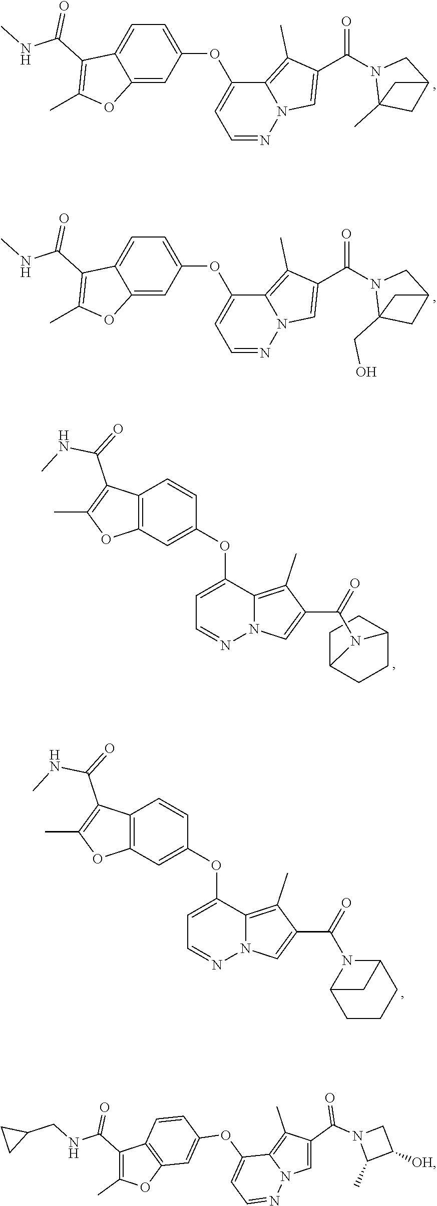 [SMILES: CNC(=O)c1c(C)oc2cc(Oc3ccnn4cc(C(=O)N5CC6CC5(C)C6)c(C)c34)ccc12 ; CNC(=O)c1c(C)oc2cc(Oc3ccnn4cc(C(=O)N5CC6CC5(CO)C6)c(C)c34)ccc12 ; CNC(=O)c1c(C)oc2cc(Oc3ccnn4cc(C(=O)N5C6CCC5CC6)c(C)c34)ccc12 ; CNC(=O)c1c(C)oc2cc(Oc3ccnn4cc(C(=O)N5C6CCCC5C6)c(C)c34)ccc12 ; Cc1oc2cc(Oc3ccnn4cc(C(=O)N5C[C@H](O)[C@@H]5C)c(C)c34)ccc2c1C(=O)NCC1CC1]

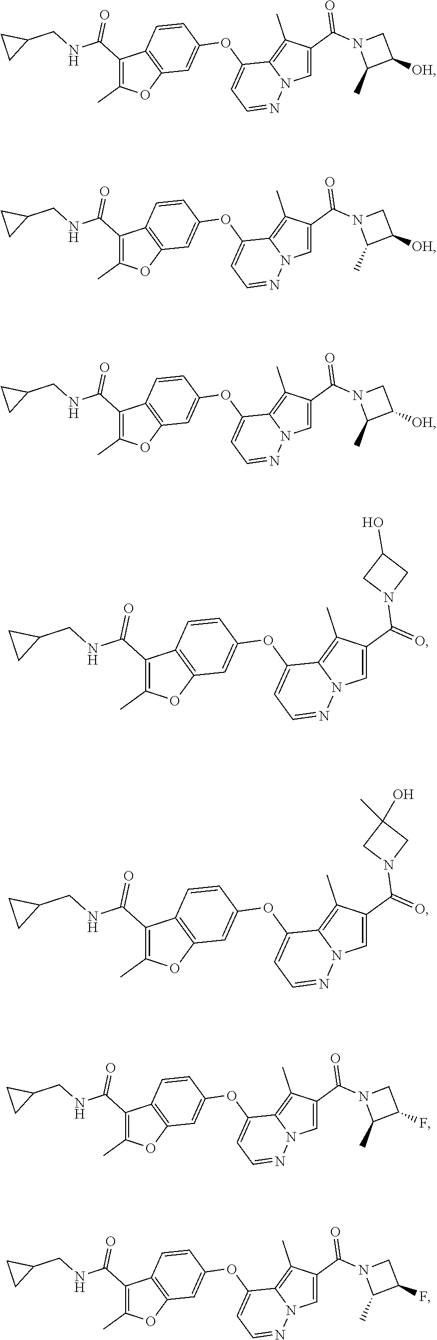

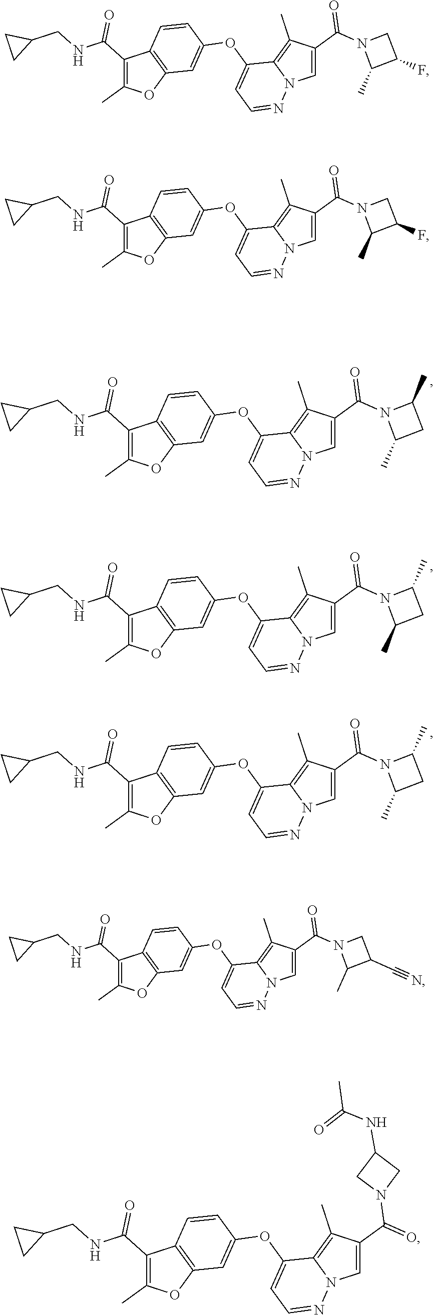

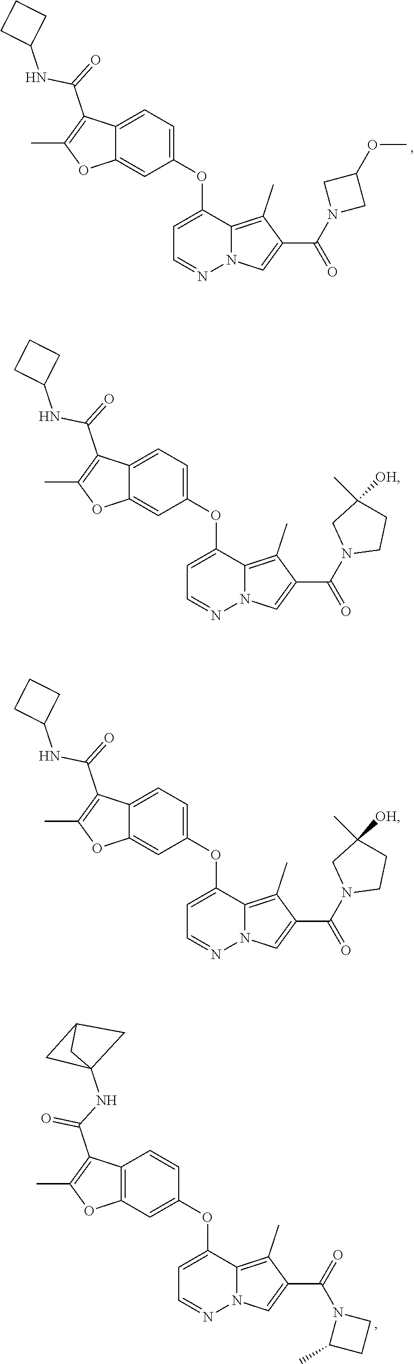

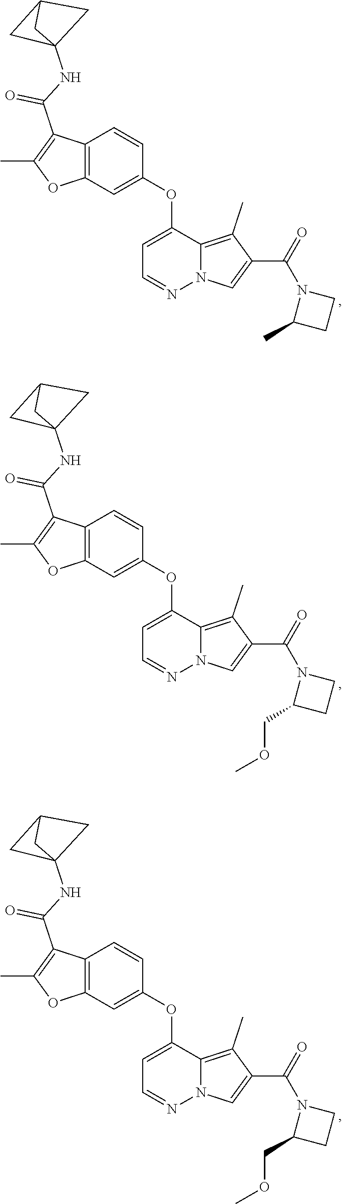 [SMILES: Cc1oc2cc(Oc3ccnn4cc(C(=O)N5CC[C@H]5C)c(C)c34)ccc2c1C(=O)NC12CC(C1)C2 ; COC[C@H]1CCN1C(=O)c1cn2nccc(Oc3ccc4c(C(=O)NC56CC(C5)C6)c(C)oc4c3)c2c1C ; COC[C@@H]1CCN1C(=O)c1cn2nccc(Oc3ccc4c(C(=O)NC56CC(C5)C6)c(C)oc4c3)c2c1C]

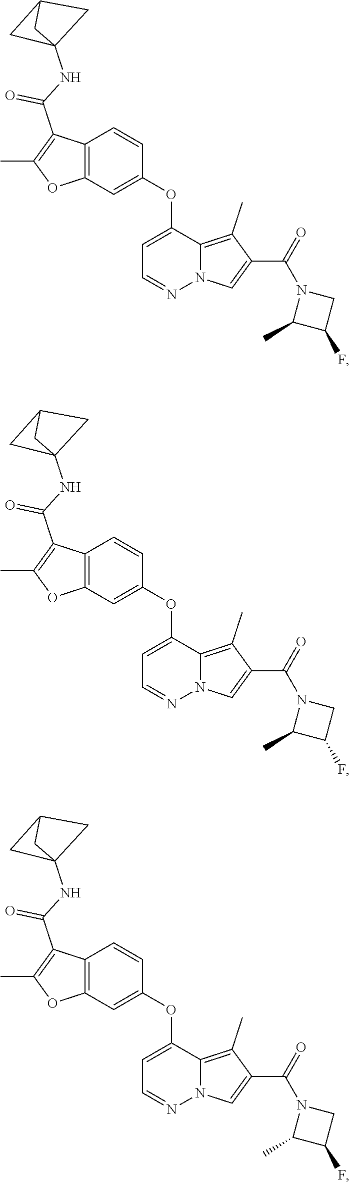 [SMILES: Cc1oc2cc(Oc3ccnn4cc(C(=O)N5C[C@@H](F)[C@H]5C)c(C)c34)ccc2c1C(=O)NC12CC(C1)C2 ; Cc1oc2cc(Oc3ccnn4cc(C(=O)N5C[C@H](F)[C@H]5C)c(C)c34)ccc2c1C(=O)NC12CC(C1)C2 ; Cc1oc2cc(Oc3ccnn4cc(C(=O)N5C[C@@H](F)[C@@H]5C)c(C)c34)ccc2c1C(=O)NC12CC(C1)C2]

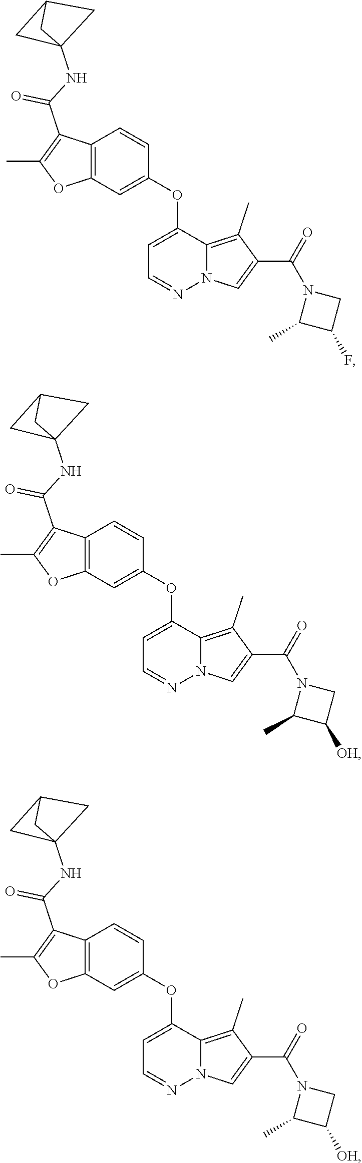 [SMILES: Cc1oc2cc(Oc3ccnn4cc(C(=O)N5C[C@H](F)[C@@H]5C)c(C)c34)ccc2c1C(=O)NC12CC(C1)C2 ; Cc1oc2cc(Oc3ccnn4cc(C(=O)N5C[C@@H](O)[C@H]5C)c(C)c34)ccc2c1C(=O)NC12CC(C1)C2 ; Cc1oc2cc(Oc3ccnn4cc(C(=O)N5C[C@H](O)[C@@H]5C)c(C)c34)ccc2c1C(=O)NC12CC(C1)C2]

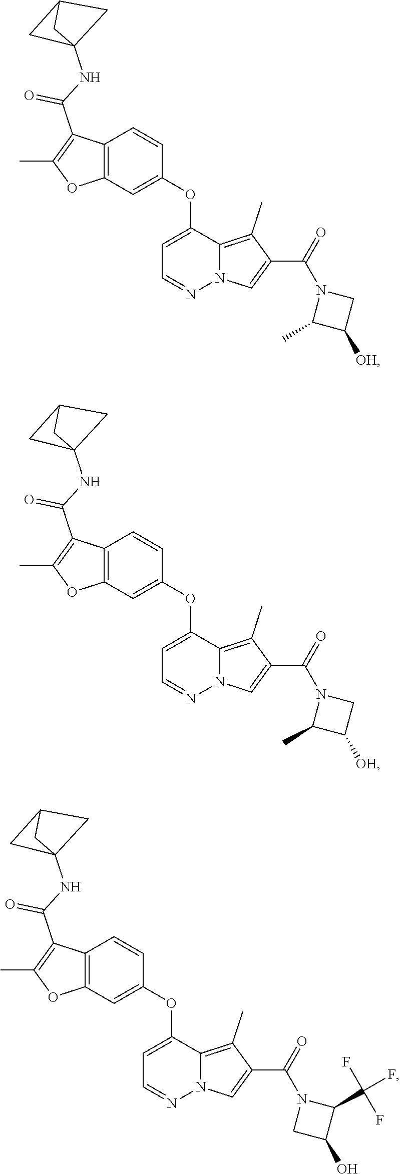 [SMILES: Cc1oc2cc(Oc3ccnn4cc(C(=O)N5C[C@@H](O)[C@@H]5C)c(C)c34)ccc2c1C(=O)NC12CC(C1)C2 ; Cc1oc2cc(Oc3ccnn4cc(C(=O)N5C[C@H](O)[C@H]5C)c(C)c34)ccc2c1C(=O)NC12CC(C1)C2 ; Cc1oc2cc(Oc3ccnn4cc(C(=O)N5C[C@H](O)[C@@H]5C(F)(F)F)c(C)c34)ccc2c1C(=O)NC12CC(C1)C2]

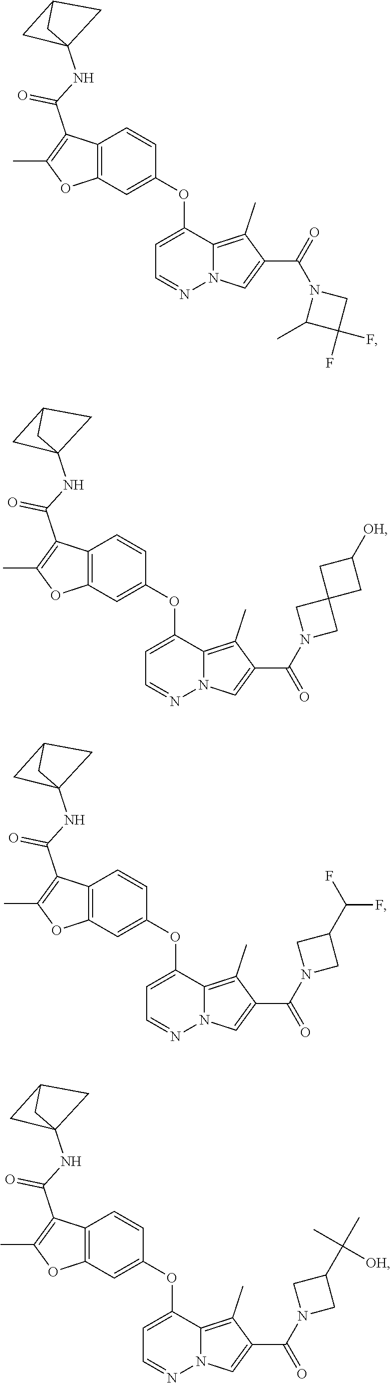 [SMILES: Cc1oc2cc(Oc3ccnn4cc(C(=O)N5CC(F)(F)C5C)c(C)c34)ccc2c1C(=O)NC12CC(C1)C2 ; Cc1oc2cc(Oc3ccnn4cc(C(=O)N5CC6(CC(O)C6)C5)c(C)c34)ccc2c1C(=O)NC12CC(C1)C2 ; Cc1oc2cc(Oc3ccnn4cc(C(=O)N5CC(C(F)F)C5)c(C)c34)ccc2c1C(=O)NC12CC(C1)C2 ; Cc1oc2cc(Oc3ccnn4cc(C(=O)N5CC(C(C)(C)O)C5)c(C)c34)ccc2c1C(=O)NC12CC(C1)C2]

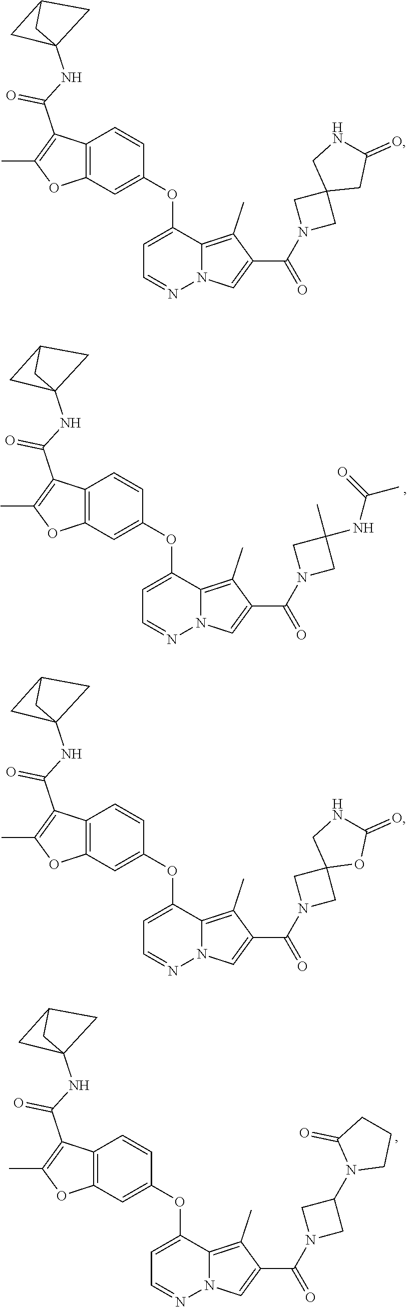

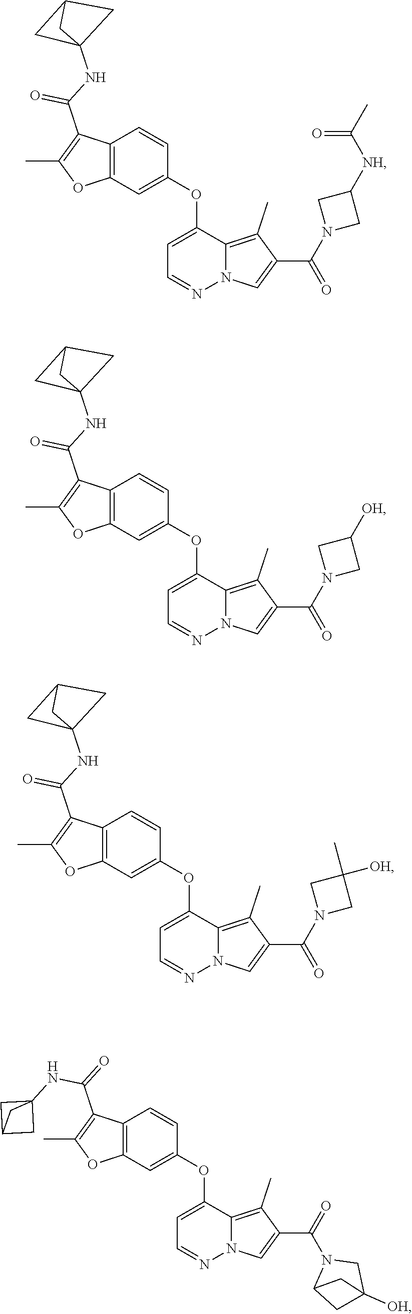

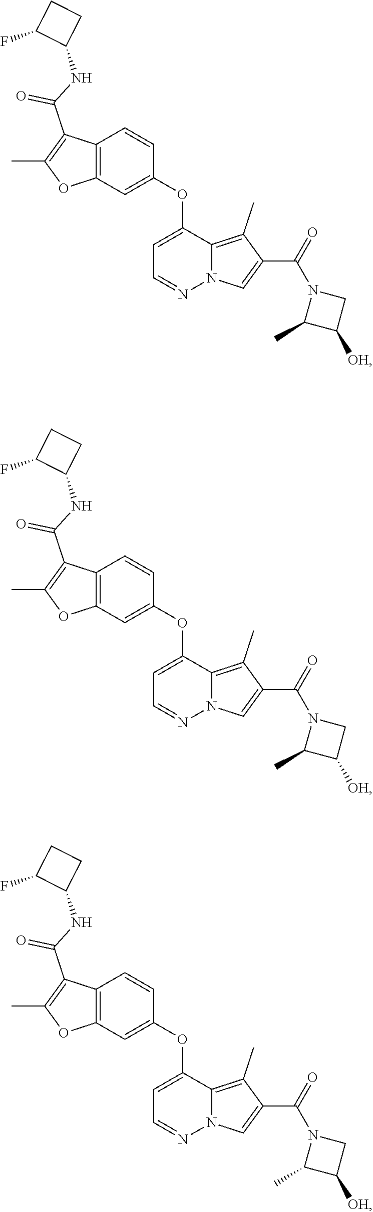 [SMILES: Cc1oc2cc(Oc3ccnn4cc(C(=O)N5C[C@@H](O)[C@H]5C)c(C)c34)ccc2c1C(=O)N[C@H]1CC[C@H]1F ; Cc1oc2cc(Oc3ccnn4cc(C(=O)N5C[C@H](O)[C@H]5C)c(C)c34)ccc2c1C(=O)N[C@H]1CC[C@H]1F ; Cc1oc2cc(Oc3ccnn4cc(C(=O)N5C[C@@H](O)[C@@H]5C)c(C)c34)ccc2c1C(=O)N[C@H]1CC[C@H]1F]

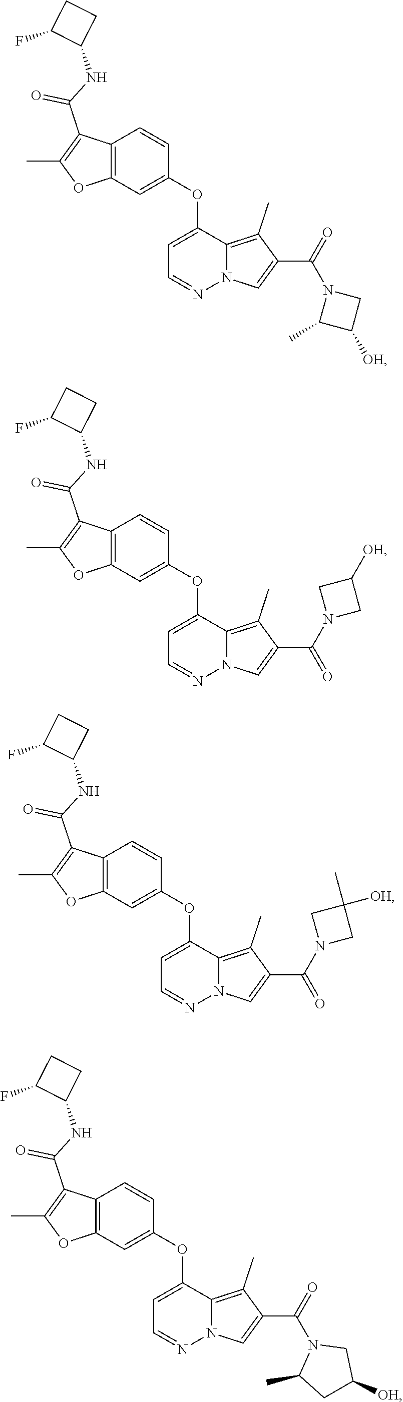 [SMILES: Cc1oc2cc(Oc3ccnn4cc(C(=O)N5C[C@H](O)[C@@H]5C)c(C)c34)ccc2c1C(=O)N[C@H]1CC[C@H]1F ; Cc1oc2cc(Oc3ccnn4cc(C(=O)N5CC(O)C5)c(C)c34)ccc2c1C(=O)N[C@H]1CC[C@H]1F ; Cc1oc2cc(Oc3ccnn4cc(C(=O)N5CC(C)(O)C5)c(C)c34)ccc2c1C(=O)N[C@H]1CC[C@H]1F ; Cc1oc2cc(Oc3ccnn4cc(C(=O)N5C[C@@H](O)C[C@H]5C)c(C)c34)ccc2c1C(=O)N[C@H]1CC[C@H]1F]

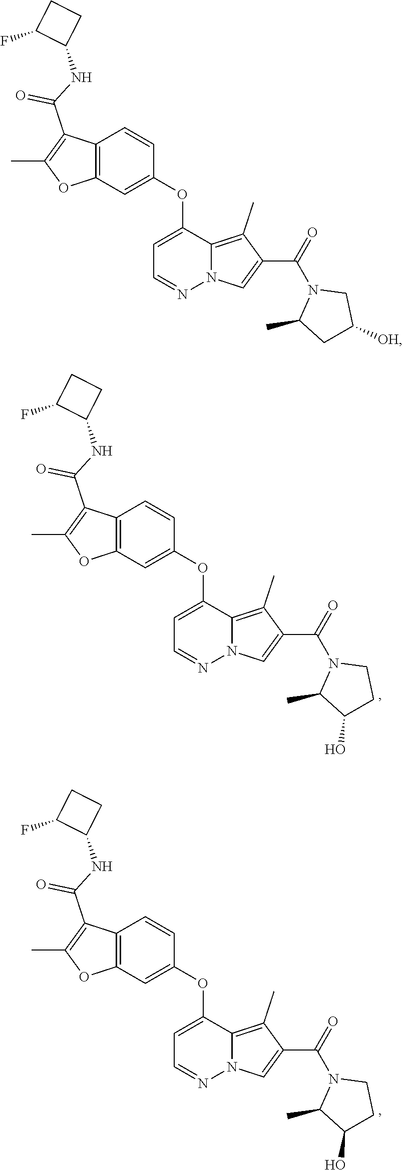 [SMILES: Cc1oc2cc(Oc3ccnn4cc(C(=O)N5C[C@H](O)C[C@H]5C)c(C)c34)ccc2c1C(=O)N[C@H]1CC[C@H]1F ; Cc1oc2cc(Oc3ccnn4cc(C(=O)N5CC[C@H](O)[C@H]5C)c(C)c34)ccc2c1C(=O)N[C@H]1CC[C@H]1F ; Cc1oc2cc(Oc3ccnn4cc(C(=O)N5CC[C@@H](O)[C@H]5C)c(C)c34)ccc2c1C(=O)N[C@H]1CC[C@H]1F]

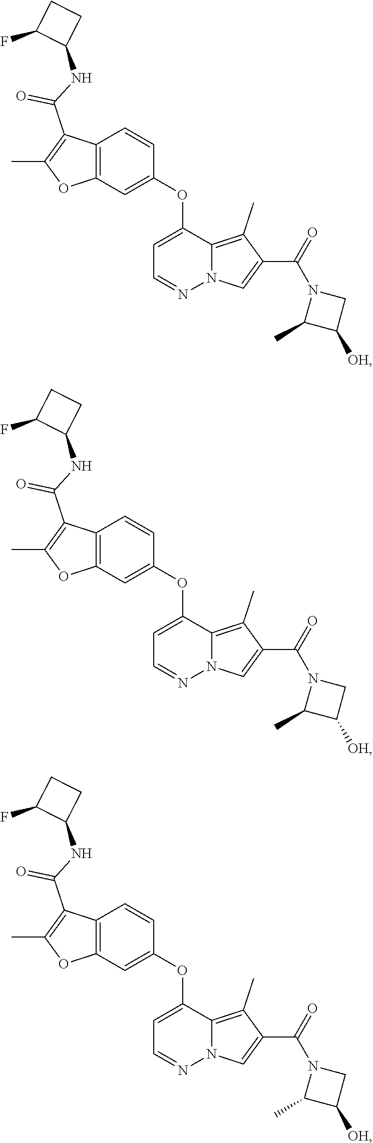 [SMILES: Cc1oc2cc(Oc3ccnn4cc(C(=O)N5C[C@@H](O)[C@H]5C)c(C)c34)ccc2c1C(=O)N[C@@H]1CC[C@@H]1F ; Cc1oc2cc(Oc3ccnn4cc(C(=O)N5C[C@H](O)[C@H]5C)c(C)c34)ccc2c1C(=O)N[C@@H]1CC[C@@H]1F ; Cc1oc2cc(Oc3ccnn4cc(C(=O)N5C[C@@H](O)[C@@H]5C)c(C)c34)ccc2c1C(=O)N[C@@H]1CC[C@@H]1F]

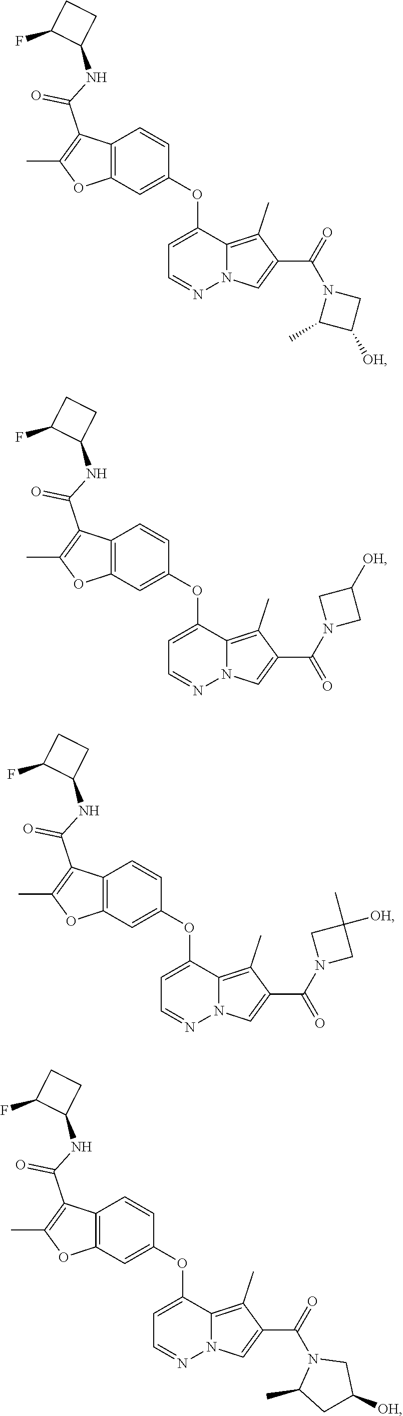 [SMILES: Cc1oc2cc(Oc3ccnn4cc(C(=O)N5C[C@H](O)[C@@H]5C)c(C)c34)ccc2c1C(=O)N[C@@H]1CC[C@@H]1F ; Cc1oc2cc(Oc3ccnn4cc(C(=O)N5CC(O)C5)c(C)c34)ccc2c1C(=O)N[C@@H]1CC[C@@H]1F ; Cc1oc2cc(Oc3ccnn4cc(C(=O)N5CC(C)(O)C5)c(C)c34)ccc2c1C(=O)N[C@@H]1CC[C@@H]1F ; Cc1oc2cc(Oc3ccnn4cc(C(=O)N5C[C@@H](O)C[C@H]5C)c(C)c34)ccc2c1C(=O)N[C@@H]1CC[C@@H]1F]

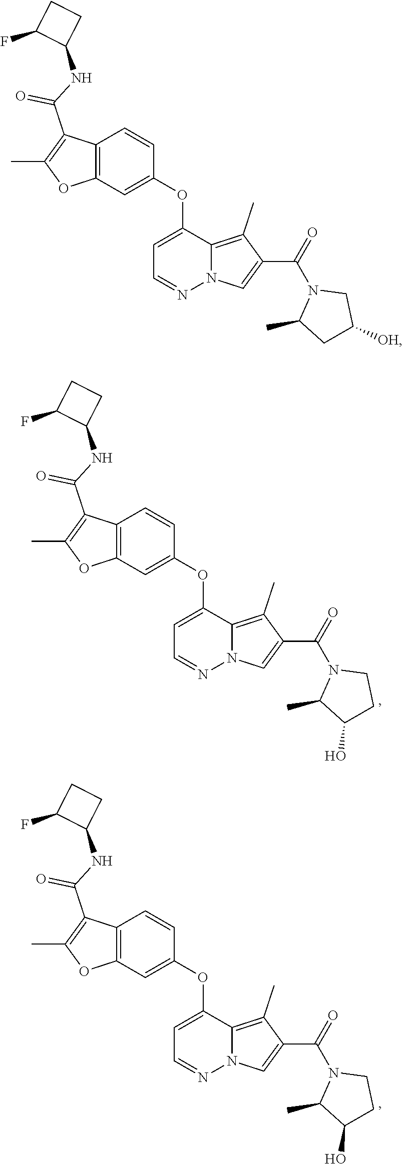 [SMILES: Cc1oc2cc(Oc3ccnn4cc(C(=O)N5C[C@H](O)C[C@H]5C)c(C)c34)ccc2c1C(=O)N[C@@H]1CC[C@@H]1F ; Cc1oc2cc(Oc3ccnn4cc(C(=O)N5CC[C@H](O)[C@H]5C)c(C)c34)ccc2c1C(=O)N[C@@H]1CC[C@@H]1F ; Cc1oc2cc(Oc3ccnn4cc(C(=O)N5CC[C@@H](O)[C@H]5C)c(C)c34)ccc2c1C(=O)N[C@@H]1CC[C@@H]1F]

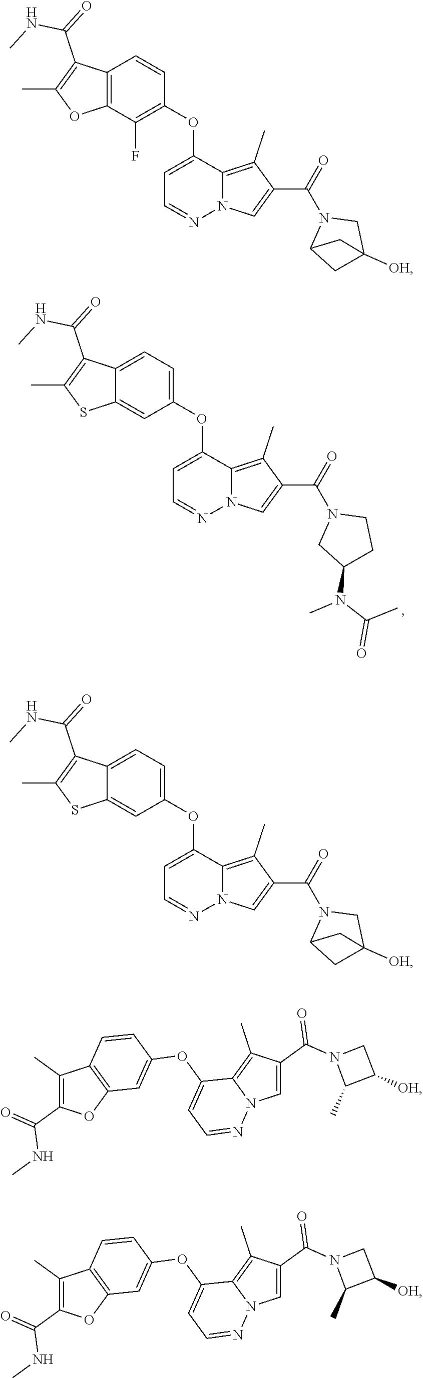

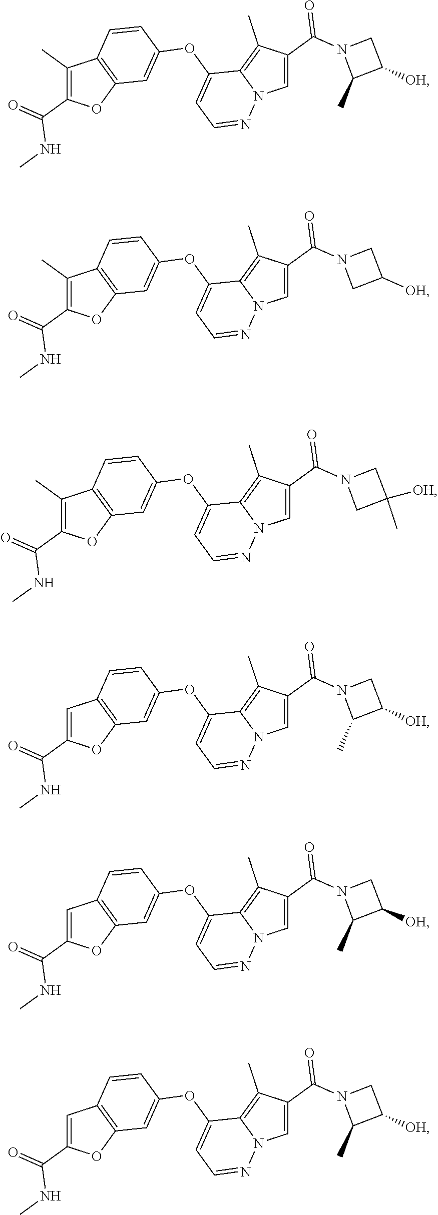 [SMILES: CNC(=O)c1oc2cc(Oc3ccnn4cc(C(=O)N5C[C@H](O)[C@H]5C)c(C)c34)ccc2c1C ; CNC(=O)c1oc2cc(Oc3ccnn4cc(C(=O)N5CC(O)C5)c(C)c34)ccc2c1C ; CNC(=O)c1oc2cc(Oc3ccnn4cc(C(=O)N5CC(C)(O)C5)c(C)c34)ccc2c1C ; CNC(=O)c1cc2ccc(Oc3ccnn4cc(C(=O)N5C[C@H](O)[C@@H]5C)c(C)c34)cc2o1 ; CNC(=O)c1cc2ccc(Oc3ccnn4cc(C(=O)N5C[C@@H](O)[C@H]5C)c(C)c34)cc2o1 ; CNC(=O)c1cc2ccc(Oc3ccnn4cc(C(=O)N5C[C@H](O)[C@H]5C)c(C)c34)cc2o1]

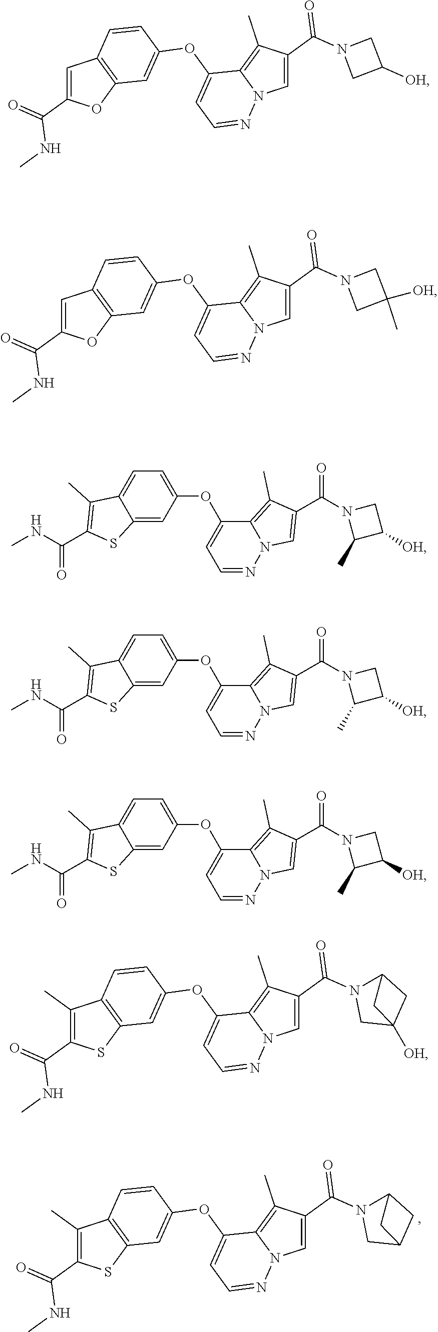

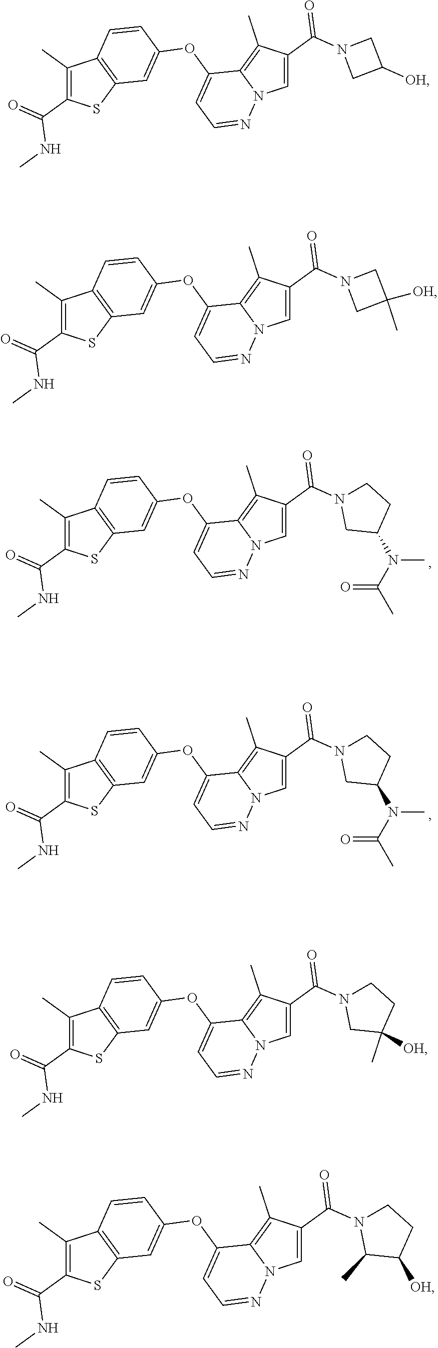 [SMILES: CNC(=O)c1sc2cc(Oc3ccnn4cc(C(=O)N5CC(O)C5)c(C)c34)ccc2c1C ; CNC(=O)c1sc2cc(Oc3ccnn4cc(C(=O)N5CC(C)(O)C5)c(C)c34)ccc2c1C ; CNC(=O)c1sc2cc(Oc3ccnn4cc(C(=O)N5CC[C@H](N(C)C(C)=O)C5)c(C)c34)ccc2c1C ; CNC(=O)c1sc2cc(Oc3ccnn4cc(C(=O)N5CC[C@@H](N(C)C(C)=O)C5)c(C)c34)ccc2c1C ; CNC(=O)c1sc2cc(Oc3ccnn4cc(C(=O)N5CC[C@@](C)(O)C5)c(C)c34)ccc2c1C ; CNC(=O)c1sc2cc(Oc3ccnn4cc(C(=O)N5CC[C@@H](O)[C@H]5C)c(C)c34)ccc2c1C]

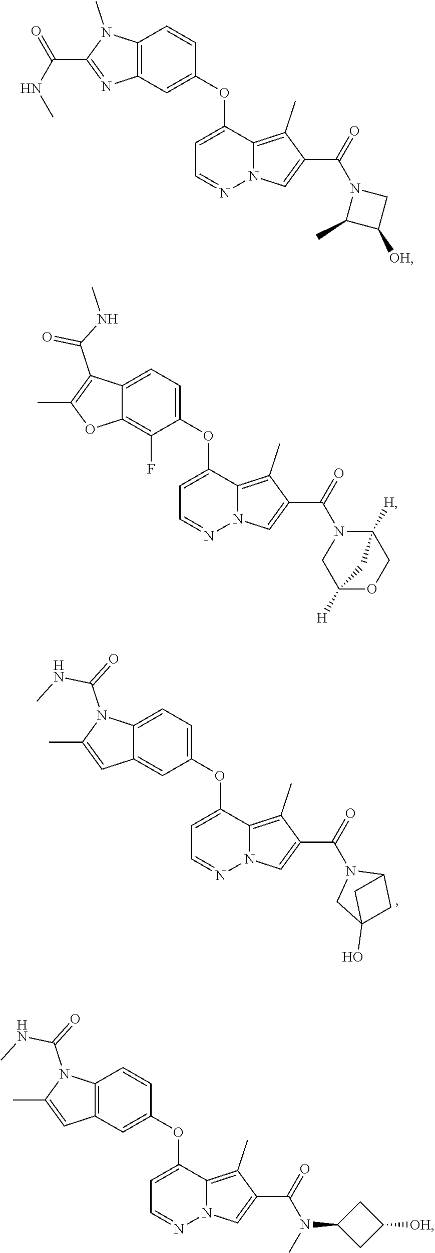

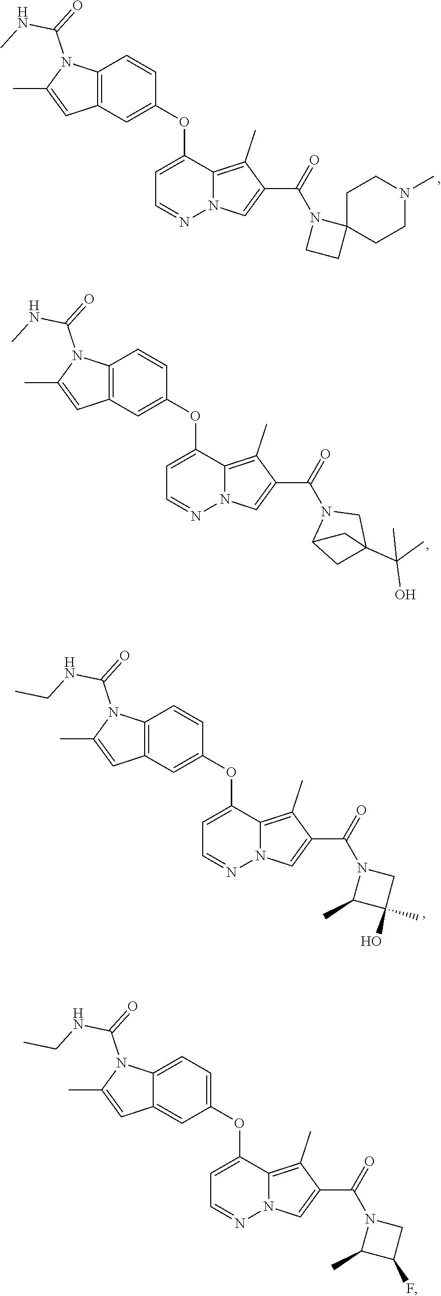

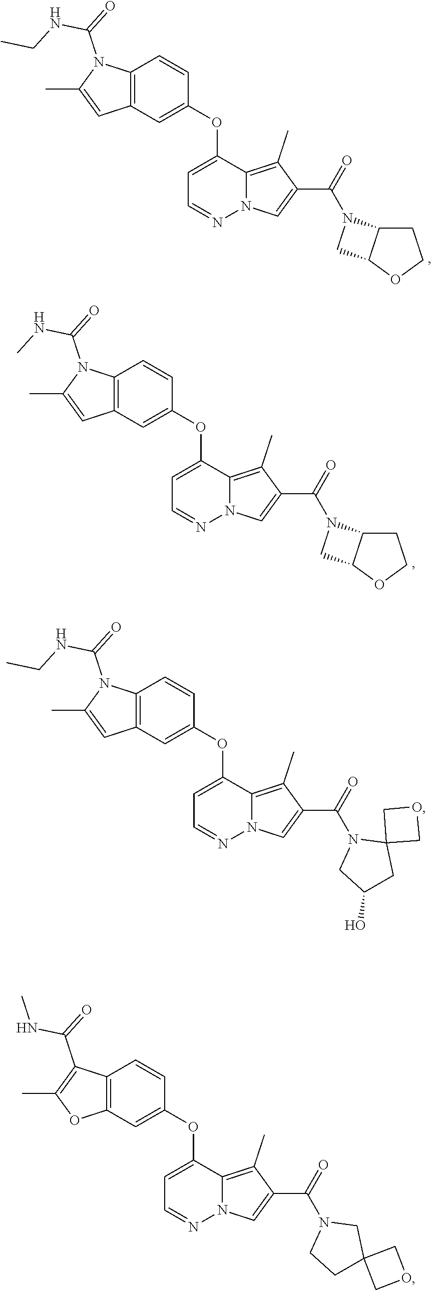 [SMILES: CCNC(=O)n1c(C)cc2cc(Oc3ccnn4cc(C(=O)N5C[C@H]6OCC[C@H]65)c(C)c34)ccc21 ; CNC(=O)n1c(C)cc2cc(Oc3ccnn4cc(C(=O)N5C[C@H]6OCC[C@H]65)c(C)c34)ccc21 ; CCNC(=O)n1c(C)cc2cc(Oc3ccnn4cc(C(=O)N5C[C@@H](O)CC56COC6)c(C)c34)ccc21 ; CNC(=O)c1c(C)oc2cc(Oc3ccnn4cc(C(=O)N5CCC6(COC6)C5)c(C)c34)ccc12]

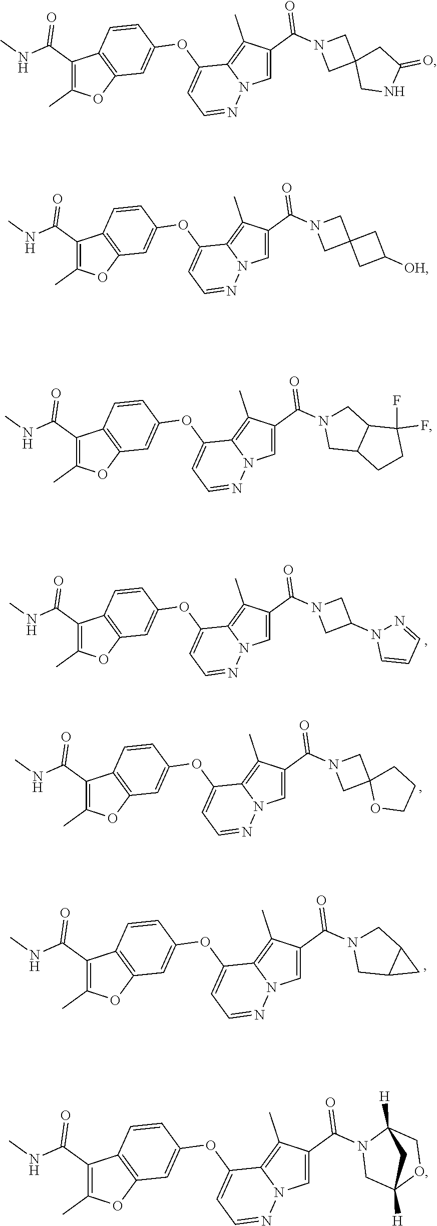

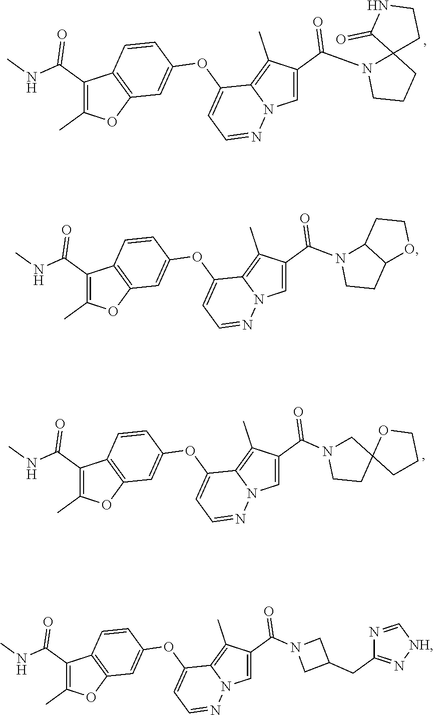 [SMILES: CNC(=O)c1c(C)oc2cc(Oc3ccnn4cc(C(=O)N5CCCC56CCNC6=O)c(C)c34)ccc12 ; CNC(=O)c1c(C)oc2cc(Oc3ccnn4cc(C(=O)N5CCC6OCCC65)c(C)c34)ccc12 ; CNC(=O)c1c(C)oc2cc(Oc3ccnn4cc(C(=O)N5CCC6(CCCO6)C5)c(C)c34)ccc12 ; CNC(=O)c1c(C)oc2cc(Oc3ccnn4cc(C(=O)N5CC(Cc6nc[nH]n6)C5)c(C)c34)ccc12]

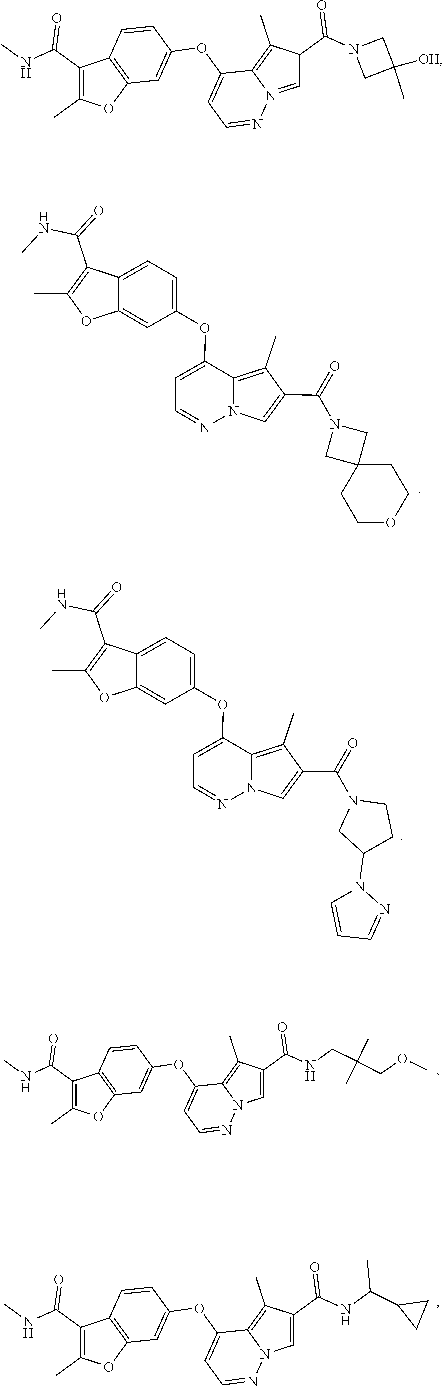

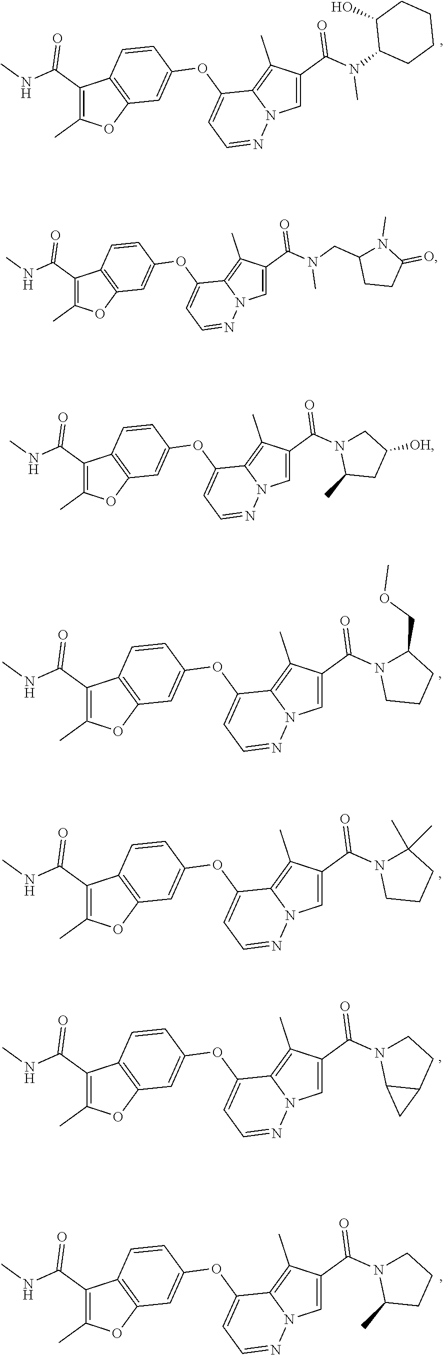

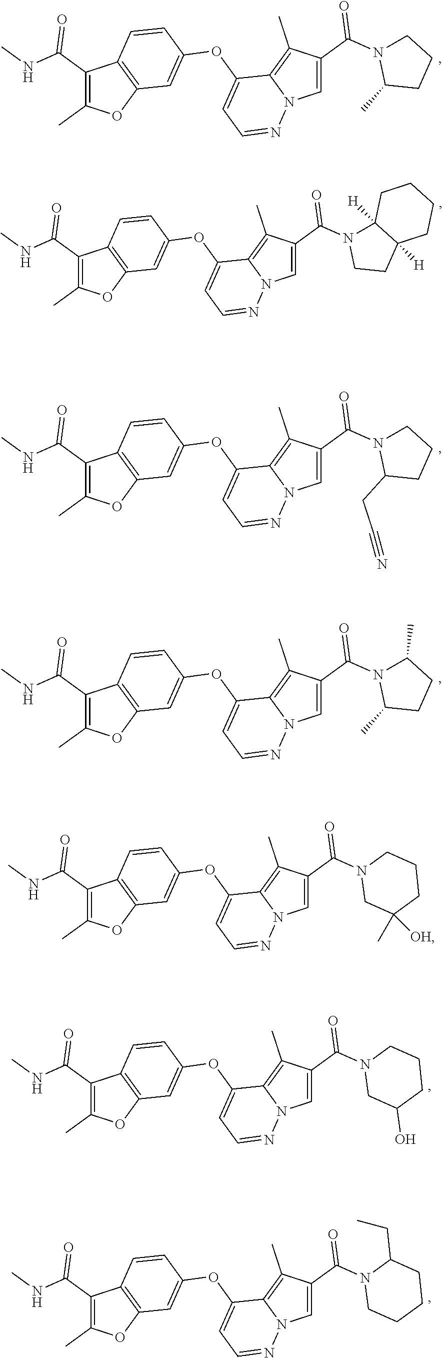

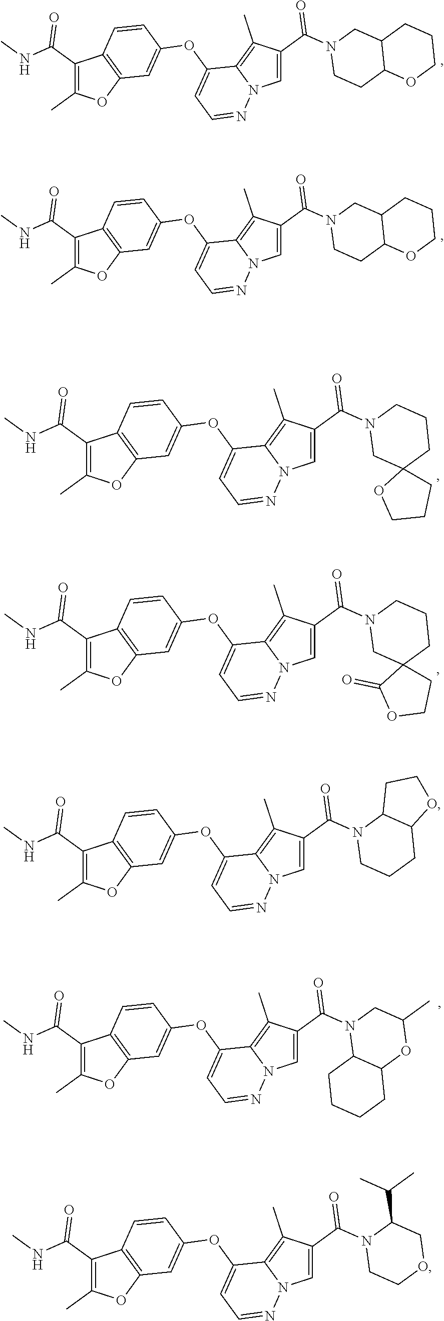

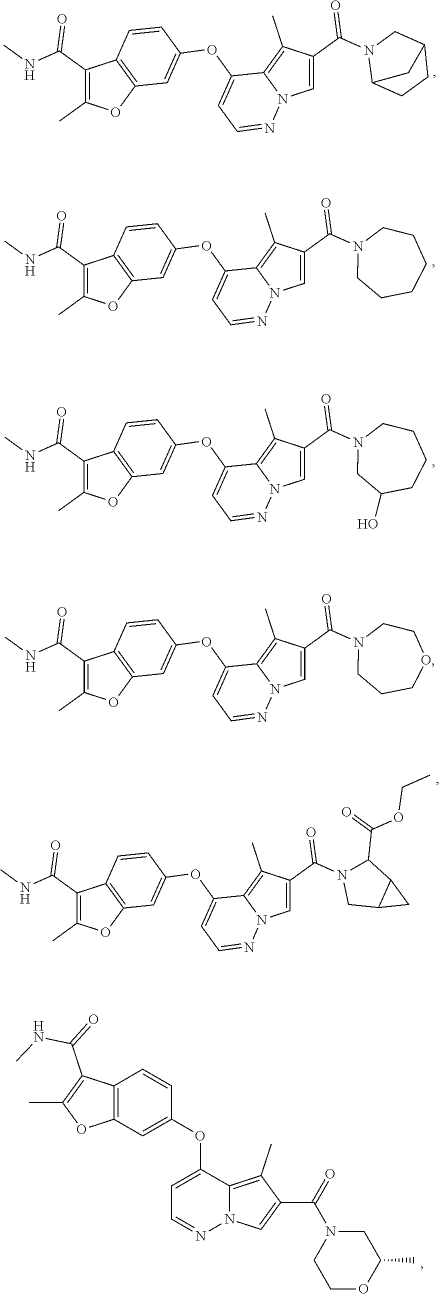

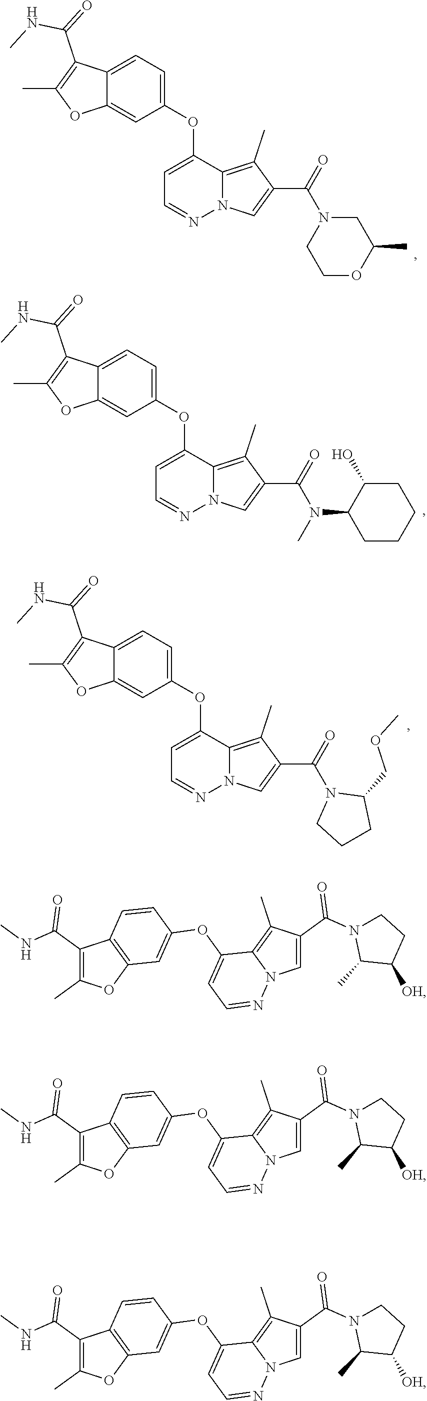

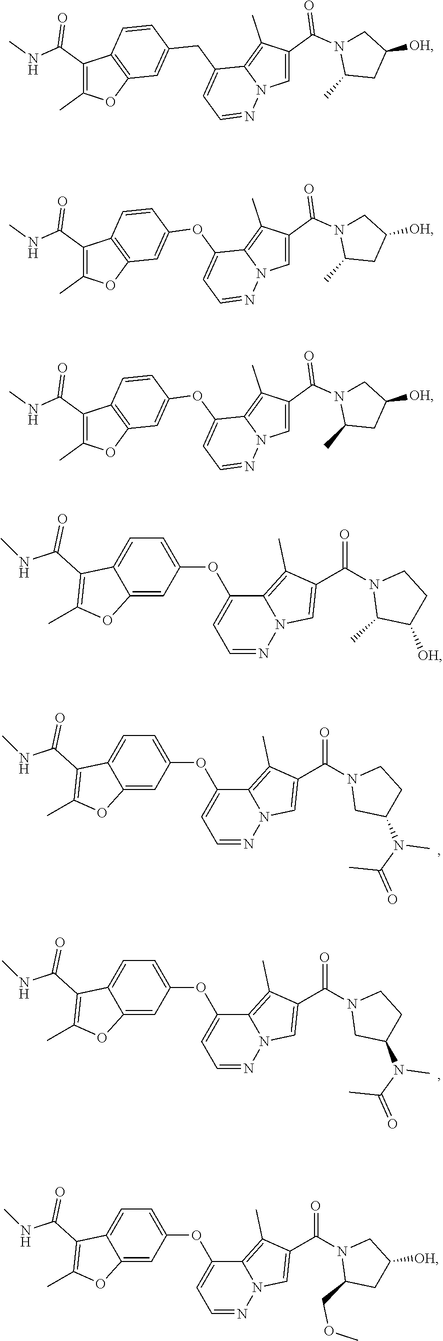

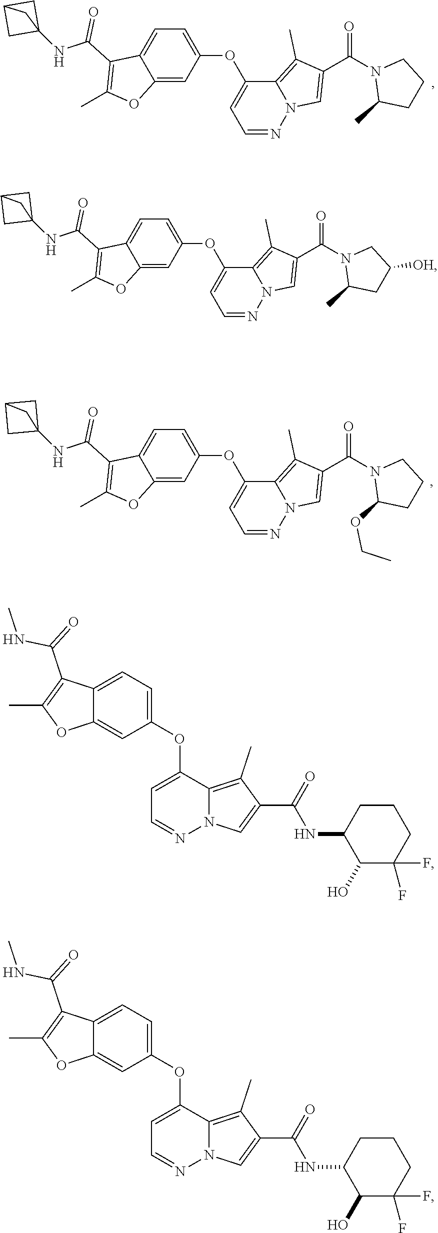

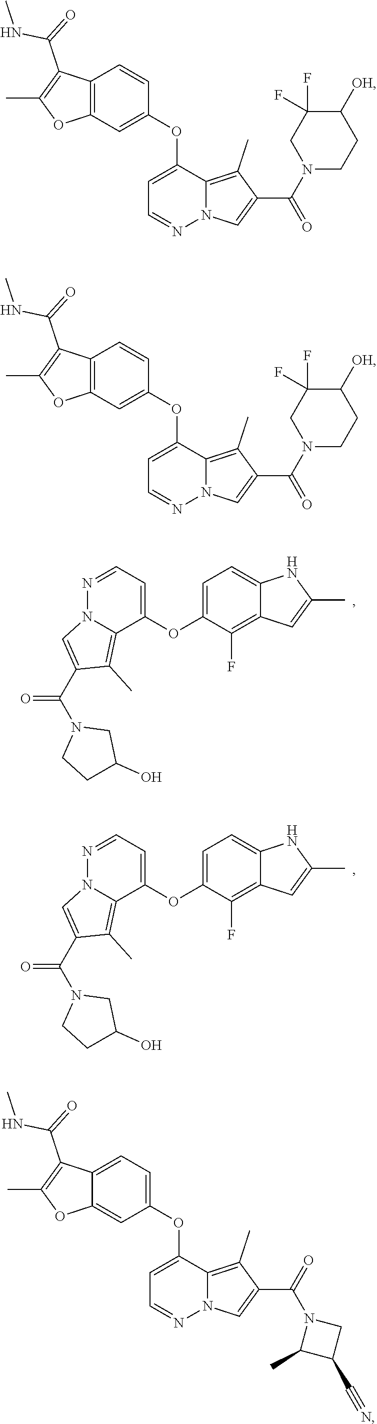

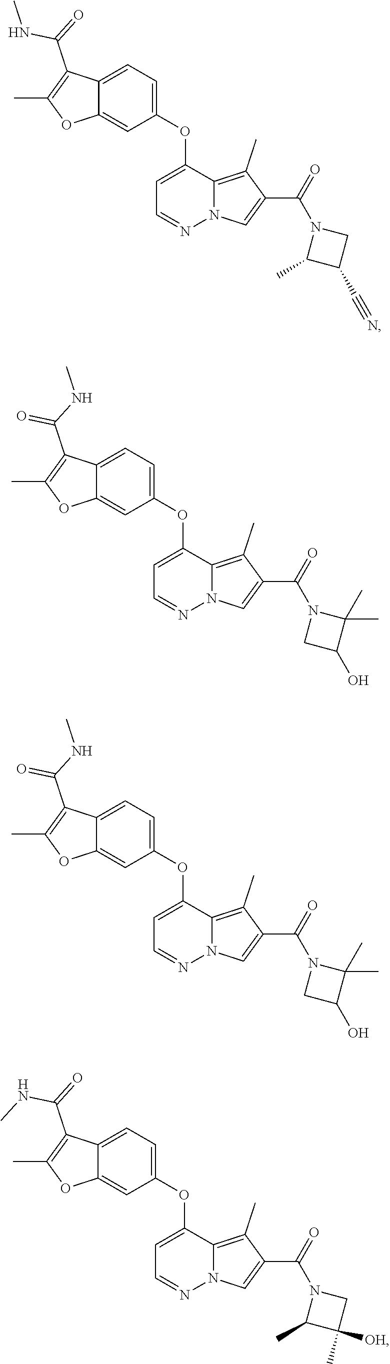

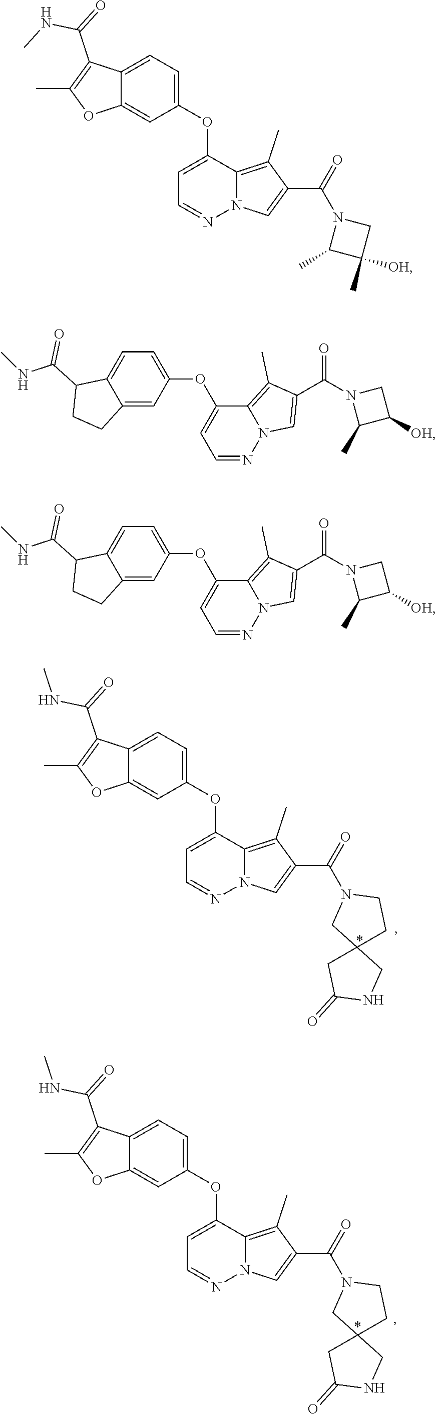 [SMILES: CNC(=O)c1c(C)oc2cc(Oc3ccnn4cc(C(=O)N5C[C@](C)(O)[C@@H]5C)c(C)c34)ccc12 ; CNC(=O)C1CCc2cc(Oc3ccnn4cc(C(=O)N5C[C@@H](O)[C@H]5C)c(C)c34)ccc21 ; CNC(=O)C1CCc2cc(Oc3ccnn4cc(C(=O)N5C[C@H](O)[C@H]5C)c(C)c34)ccc21 ; CNC(=O)c1c(C)oc2cc(Oc3ccnn4cc(C(=O)N5CCC6(CNC(=O)C6)C5)c(C)c34)ccc12 ; CNC(=O)c1c(C)oc2cc(Oc3ccnn4cc(C(=O)N5CCC6(CNC(=O)C6)C5)c(C)c34)ccc12]

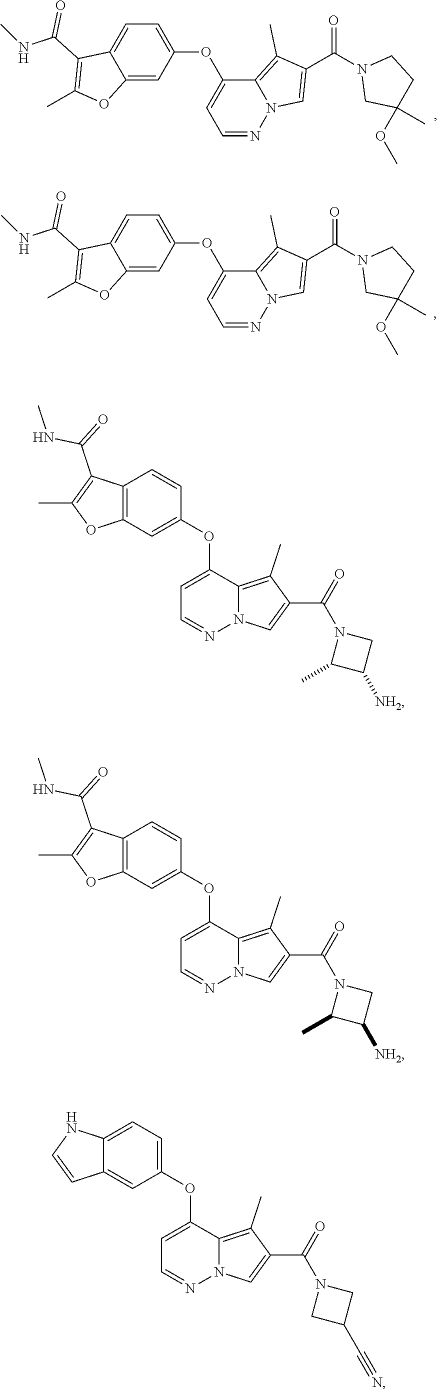

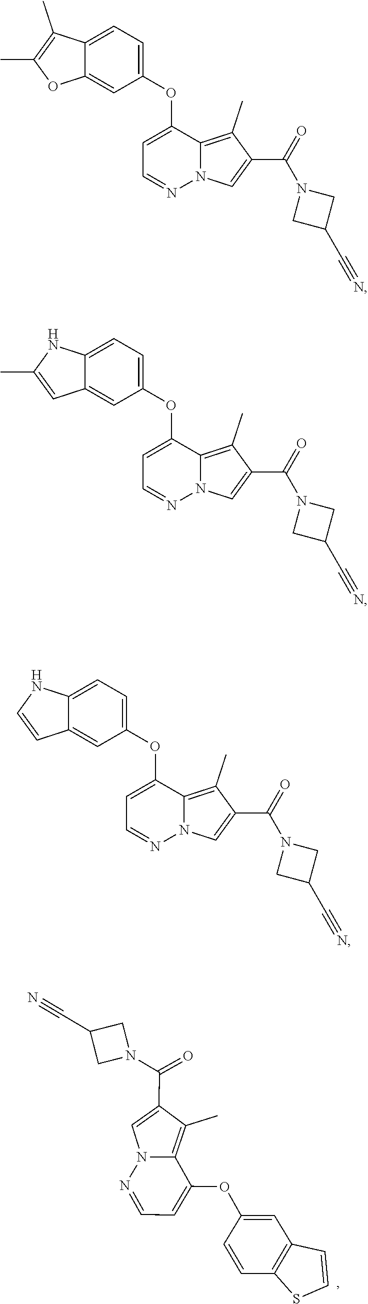 [SMILES: Cc1oc2cc(Oc3ccnn4cc(C(=O)N5CC(C#N)C5)c(C)c34)ccc2c1C ; Cc1cc2cc(Oc3ccnn4cc(C(=O)N5CC(C#N)C5)c(C)c34)ccc2[nH]1 ; Cc1c(C(=O)N2CC(C#N)C2)cn2nccc(Oc3ccc4[nH]ccc4c3)c12 ; Cc1c(C(=O)N2CC(C#N)C2)cn2nccc(Oc3ccc4sccc4c3)c12]

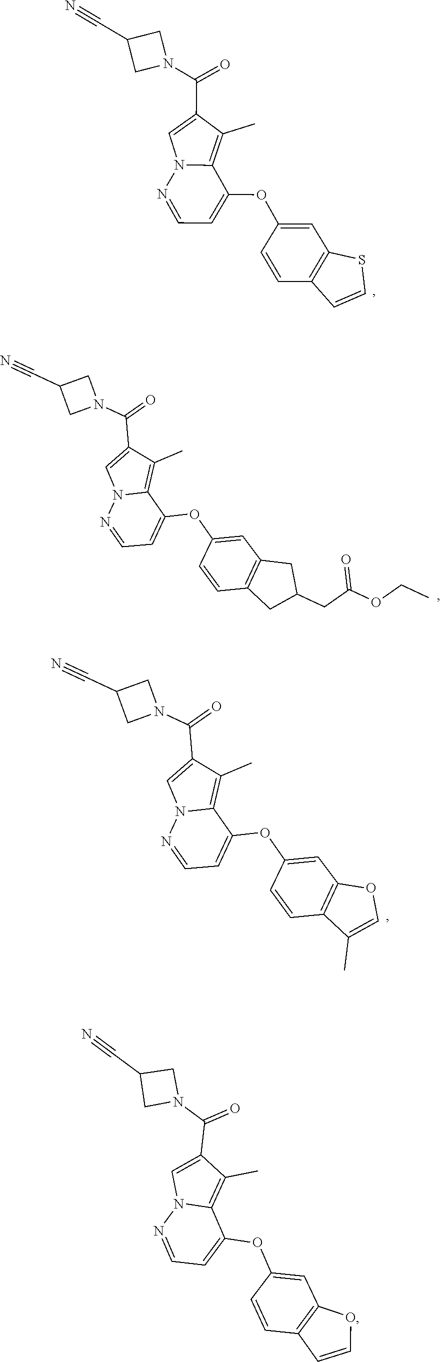 [SMILES: Cc1c(C(=O)N2CC(C#N)C2)cn2nccc(Oc3ccc4ccsc4c3)c12 ; CCOC(=O)CC1Cc2ccc(Oc3ccnn4cc(C(=O)N5CC(C#N)C5)c(C)c34)cc2C1 ; Cc1coc2cc(Oc3ccnn4cc(C(=O)N5CC(C#N)C5)c(C)c34)ccc12 ; Cc1c(C(=O)N2CC(C#N)C2)cn2nccc(Oc3ccc4ccoc4c3)c12]

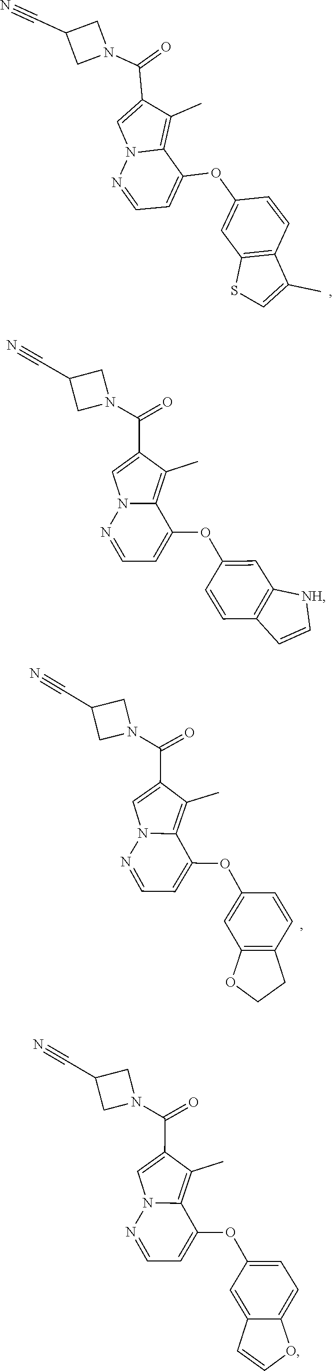 [SMILES: Cc1csc2cc(Oc3ccnn4cc(C(=O)N5CC(C#N)C5)c(C)c34)ccc12 ; Cc1c(C(=O)N2CC(C#N)C2)cn2nccc(Oc3ccc4cc[nH]c4c3)c12 ; Cc1c(C(=O)N2CC(C#N)C2)cn2nccc(Oc3ccc4c(c3)OCC4)c12 ; Cc1c(C(=O)N2CC(C#N)C2)cn2nccc(Oc3ccc4occc4c3)c12]

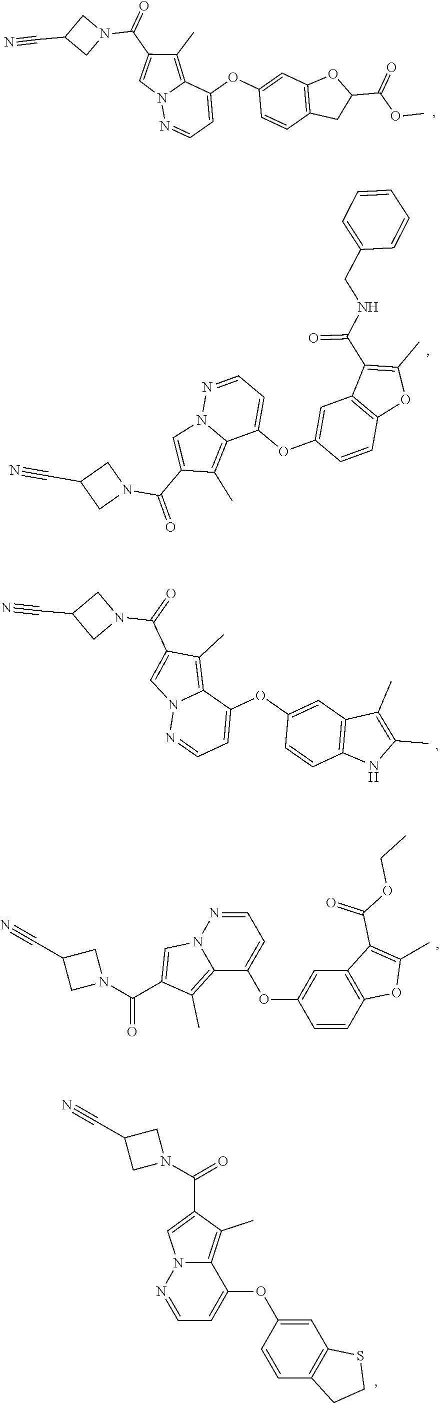 [SMILES: COC(=O)C1Cc2ccc(Oc3ccnn4cc(C(=O)N5CC(C#N)C5)c(C)c34)cc2O1 ; Cc1oc2ccc(Oc3ccnn4cc(C(=O)N5CC(C#N)C5)c(C)c34)cc2c1C(=O)NCc1ccccc1 ; Cc1[nH]c2ccc(Oc3ccnn4cc(C(=O)N5CC(C#N)C5)c(C)c34)cc2c1C ; CCOC(=O)c1c(C)oc2ccc(Oc3ccnn4cc(C(=O)N5CC(C#N)C5)c(C)c34)cc12 ; Cc1c(C(=O)N2CC(C#N)C2)cn2nccc(Oc3ccc4c(c3)SCC4)c12]

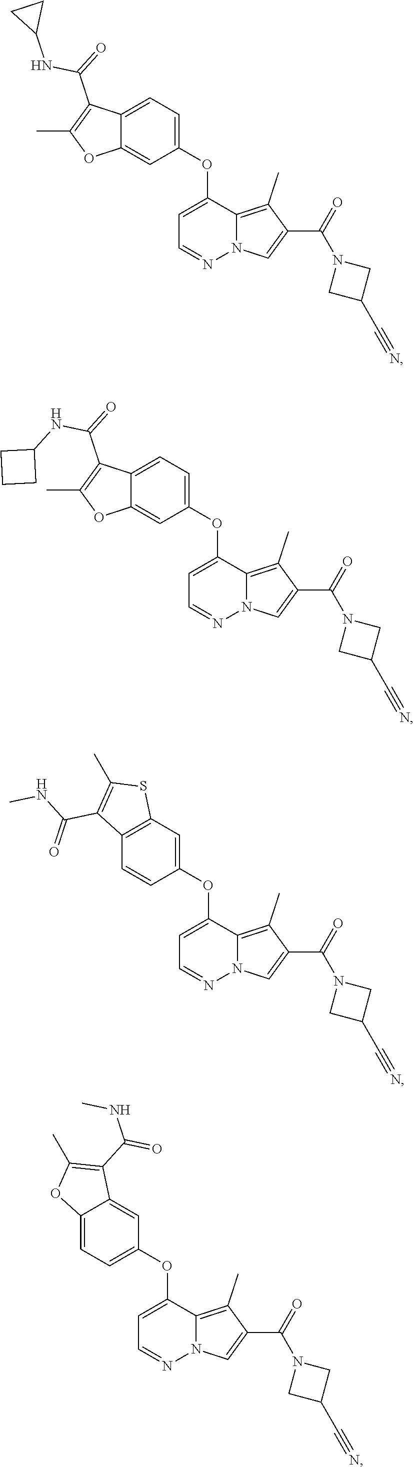 [SMILES: Cc1oc2cc(Oc3ccnn4cc(C(=O)N5CC(C#N)C5)c(C)c34)ccc2c1C(=O)NC1CC1 ; Cc1oc2cc(Oc3ccnn4cc(C(=O)N5CC(C#N)C5)c(C)c34)ccc2c1C(=O)NC1CCC1 ; CNC(=O)c1c(C)sc2cc(Oc3ccnn4cc(C(=O)N5CC(C#N)C5)c(C)c34)ccc12 ; CNC(=O)c1c(C)oc2ccc(Oc3ccnn4cc(C(=O)N5CC(C#N)C5)c(C)c34)cc12]

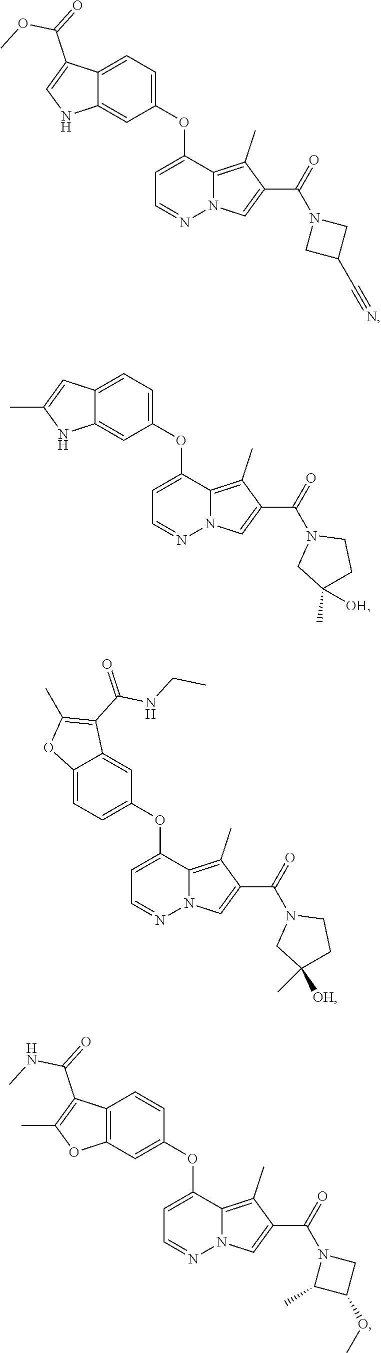 [SMILES: COC(=O)c1c[nH]c2cc(Oc3ccnn4cc(C(=O)N5CC(C#N)C5)c(C)c34)ccc12 ; Cc1cc2ccc(Oc3ccnn4cc(C(=O)N5CC[C@@](C)(O)C5)c(C)c34)cc2[nH]1 ; CCNC(=O)c1c(C)oc2ccc(Oc3ccnn4cc(C(=O)N5CC[C@@](C)(O)C5)c(C)c34)cc12 ; CNC(=O)c1c(C)oc2cc(Oc3ccnn4cc(C(=O)N5C[C@H](OC)[C@@H]5C)c(C)c34)ccc12]

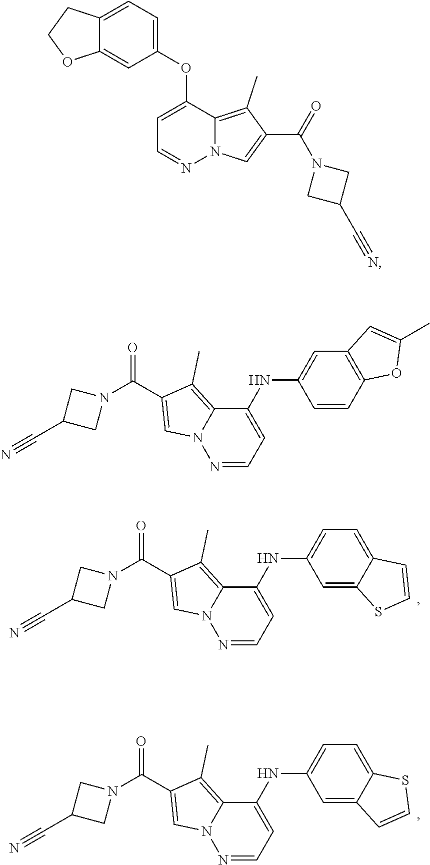 [SMILES: Cc1c(C(=O)N2CC(C#N)C2)cn2nccc(Oc3ccc4c(c3)OCC4)c12 ; Cc1cc2cc(Nc3ccnn4cc(C(=O)N5CC(C#N)C5)c(C)c34)ccc2o1 ; Cc1c(C(=O)N2CC(C#N)C2)cn2nccc(Nc3ccc4ccsc4c3)c12 ; Cc1c(C(=O)N2CC(C#N)C2)cn2nccc(Nc3ccc4sccc4c3)c12]

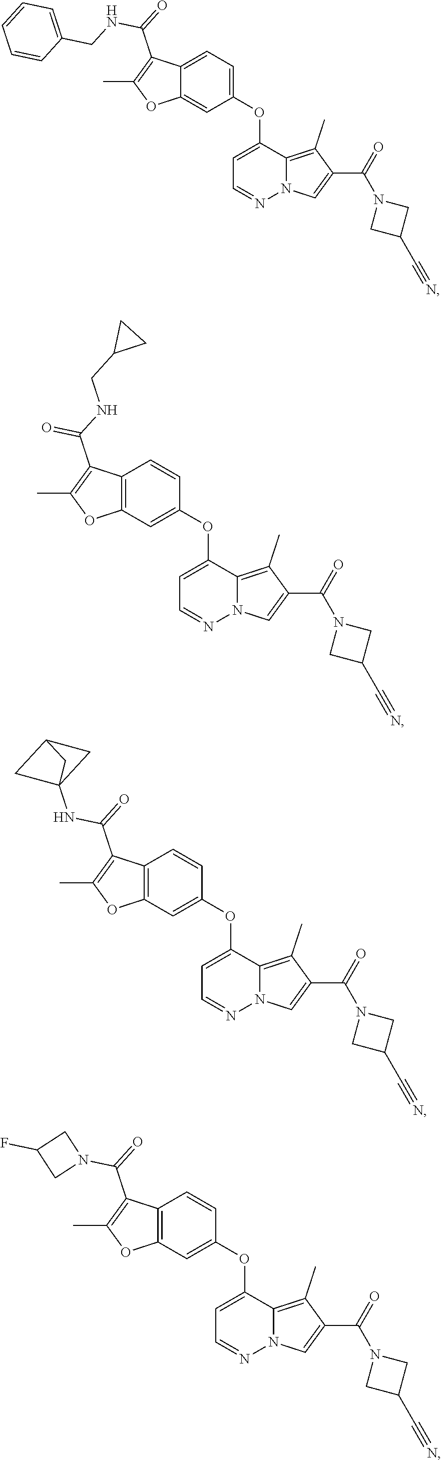 [SMILES: Cc1oc2cc(Oc3ccnn4cc(C(=O)N5CC(C#N)C5)c(C)c34)ccc2c1C(=O)NCc1ccccc1 ; Cc1oc2cc(Oc3ccnn4cc(C(=O)N5CC(C#N)C5)c(C)c34)ccc2c1C(=O)NCC1CC1 ; Cc1oc2cc(Oc3ccnn4cc(C(=O)N5CC(C#N)C5)c(C)c34)ccc2c1C(=O)NC12CC(C1)C2 ; Cc1oc2cc(Oc3ccnn4cc(C(=O)N5CC(C#N)C5)c(C)c34)ccc2c1C(=O)N1CC(F)C1]

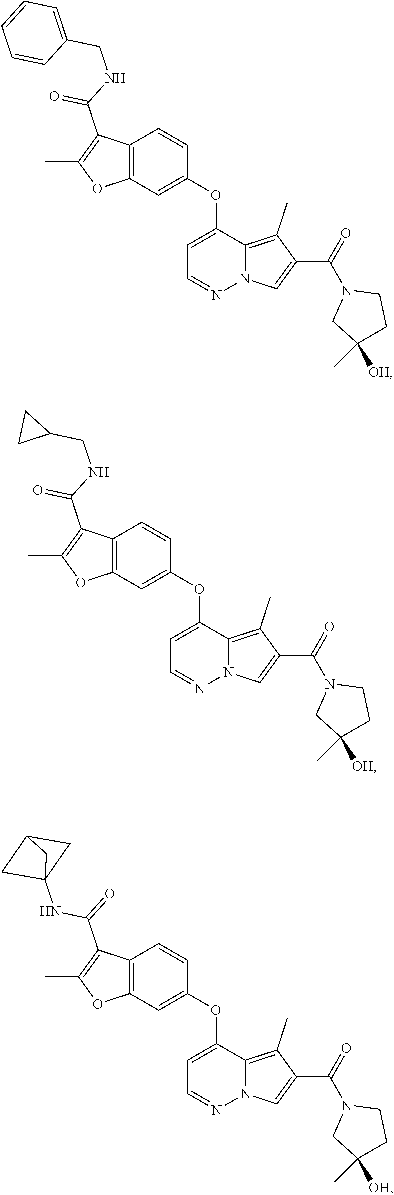 [SMILES: Cc1oc2cc(Oc3ccnn4cc(C(=O)N5CC[C@@](C)(O)C5)c(C)c34)ccc2c1C(=O)NCc1ccccc1 ; Cc1oc2cc(Oc3ccnn4cc(C(=O)N5CC[C@@](C)(O)C5)c(C)c34)ccc2c1C(=O)NCC1CC1 ; Cc1oc2cc(Oc3ccnn4cc(C(=O)N5CC[C@@](C)(O)C5)c(C)c34)ccc2c1C(=O)NC12CC(C1)C2]

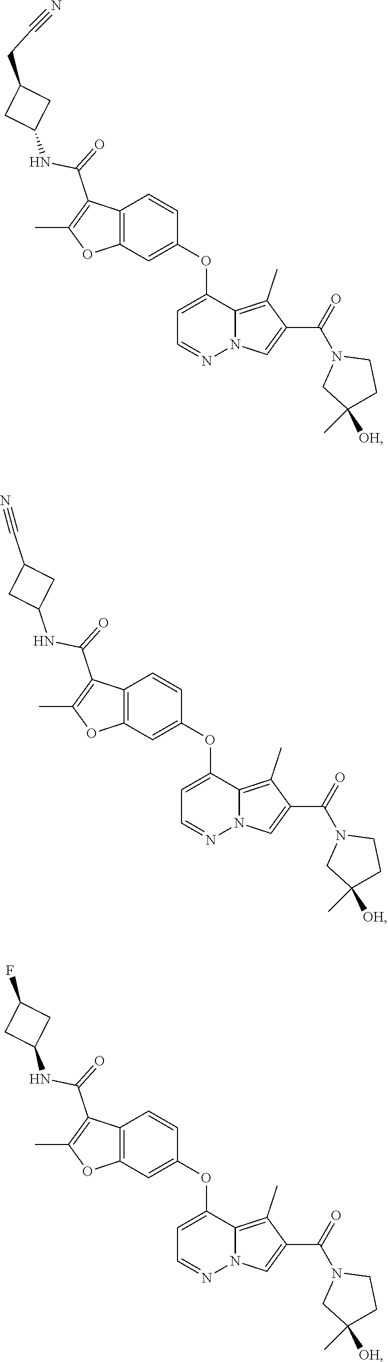 [SMILES: Cc1oc2cc(Oc3ccnn4cc(C(=O)N5CC[C@@](C)(O)C5)c(C)c34)ccc2c1C(=O)N[C@H]1C[C@H](CC#N)C1 ; Cc1oc2cc(Oc3ccnn4cc(C(=O)N5CC[C@@](C)(O)C5)c(C)c34)ccc2c1C(=O)NC1CC(C#N)C1 ; Cc1oc2cc(Oc3ccnn4cc(C(=O)N5CC[C@@](C)(O)C5)c(C)c34)ccc2c1C(=O)N[C@H]1C[C@@H](F)C1]

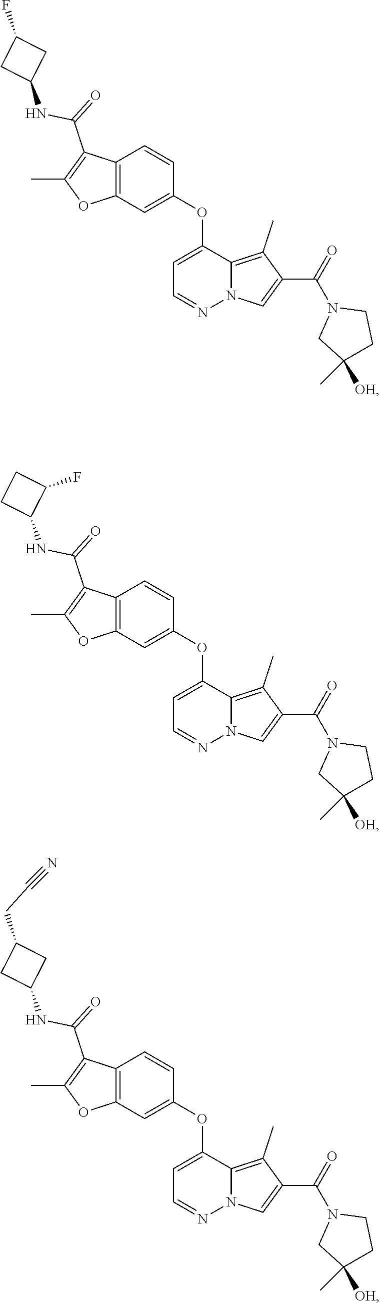 [SMILES: Cc1oc2cc(Oc3ccnn4cc(C(=O)N5CC[C@@](C)(O)C5)c(C)c34)ccc2c1C(=O)N[C@H]1C[C@H](F)C1 ; Cc1oc2cc(Oc3ccnn4cc(C(=O)N5CC[C@@](C)(O)C5)c(C)c34)ccc2c1C(=O)N[C@@H]1CC[C@@H]1F ; Cc1oc2cc(Oc3ccnn4cc(C(=O)N5CC[C@@](C)(O)C5)c(C)c34)ccc2c1C(=O)N[C@H]1C[C@@H](CC#N)C1]

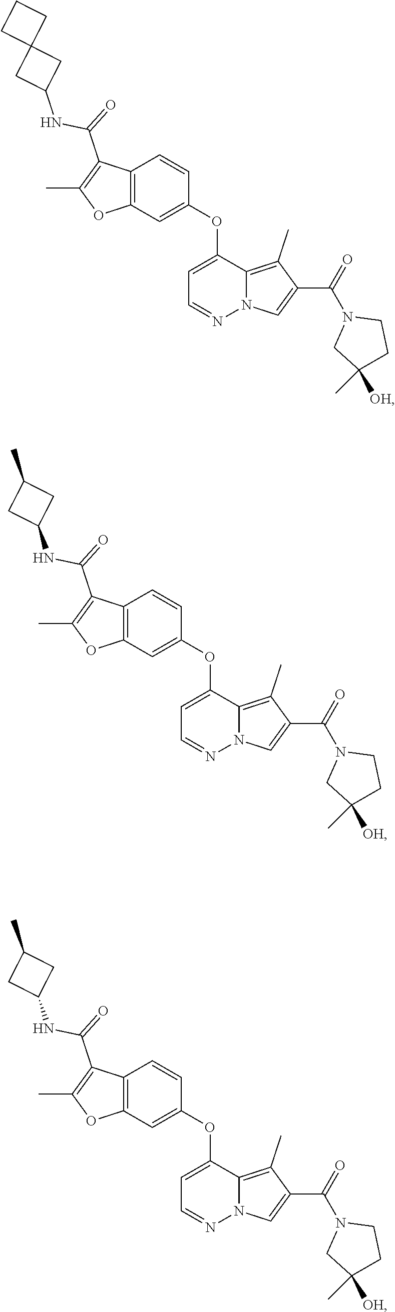 [SMILES: Cc1oc2cc(Oc3ccnn4cc(C(=O)N5CC[C@@](C)(O)C5)c(C)c34)ccc2c1C(=O)NC1CC2(CCC2)C1 ; Cc1oc2cc(Oc3ccnn4cc(C(=O)N5CC[C@@](C)(O)C5)c(C)c34)ccc2c1C(=O)N[C@H]1C[C@@H](C)C1 ; Cc1oc2cc(Oc3ccnn4cc(C(=O)N5CC[C@@](C)(O)C5)c(C)c34)ccc2c1C(=O)N[C@H]1C[C@H](C)C1]

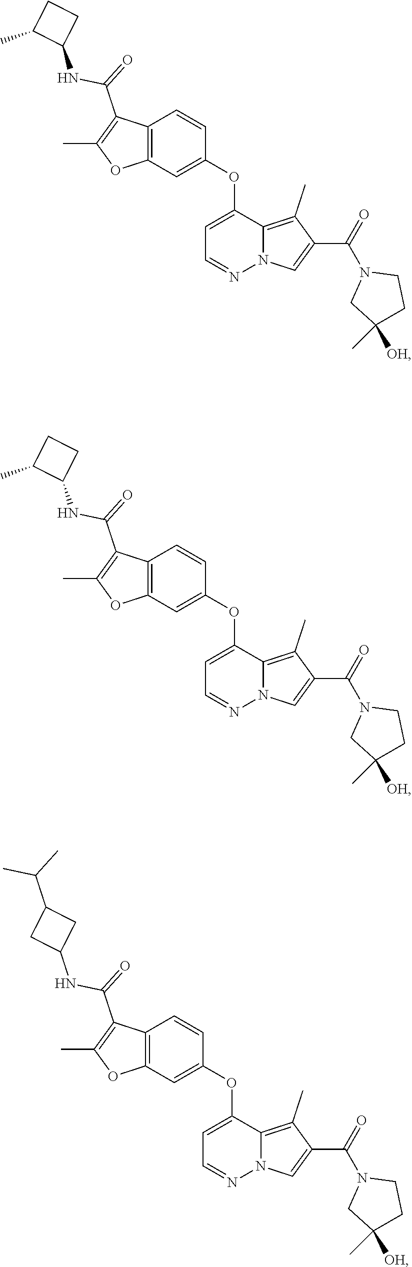 [SMILES: Cc1oc2cc(Oc3ccnn4cc(C(=O)N5CC[C@@](C)(O)C5)c(C)c34)ccc2c1C(=O)N[C@@H]1CC[C@H]1C ; Cc1oc2cc(Oc3ccnn4cc(C(=O)N5CC[C@@](C)(O)C5)c(C)c34)ccc2c1C(=O)N[C@H]1CC[C@H]1C ; Cc1oc2cc(Oc3ccnn4cc(C(=O)N5CC[C@@](C)(O)C5)c(C)c34)ccc2c1C(=O)NC1CC(C(C)C)C1]

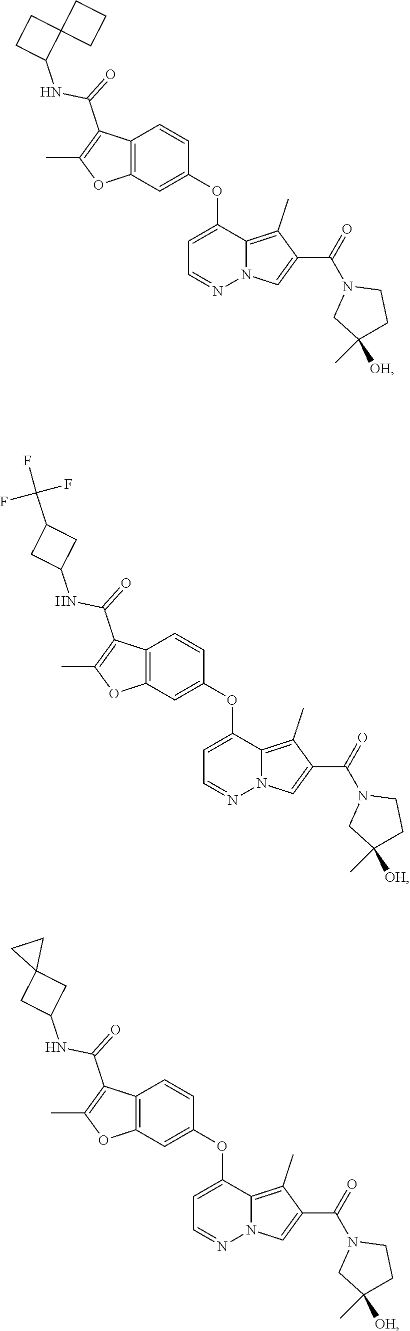 [SMILES: Cc1oc2cc(Oc3ccnn4cc(C(=O)N5CC[C@@](C)(O)C5)c(C)c34)ccc2c1C(=O)NC1CCC12CCC2 ; Cc1oc2cc(Oc3ccnn4cc(C(=O)N5CC[C@@](C)(O)C5)c(C)c34)ccc2c1C(=O)NC1CC(C(F)(F)F)C1 ; Cc1oc2cc(Oc3ccnn4cc(C(=O)N5CC[C@@](C)(O)C5)c(C)c34)ccc2c1C(=O)NC1CC2(CC2)C1]

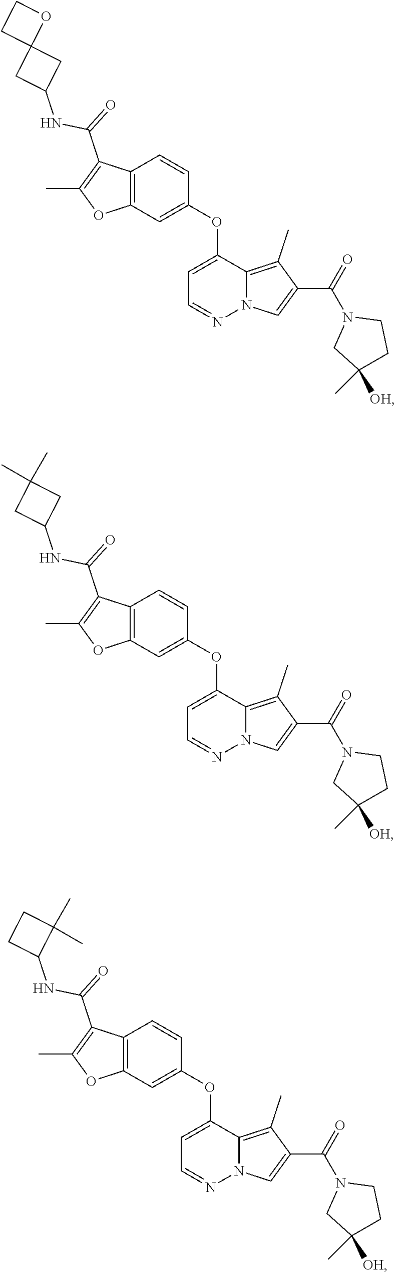 [SMILES: Cc1oc2cc(Oc3ccnn4cc(C(=O)N5CC[C@@](C)(O)C5)c(C)c34)ccc2c1C(=O)NC1CC2(CCO2)C1 ; Cc1oc2cc(Oc3ccnn4cc(C(=O)N5CC[C@@](C)(O)C5)c(C)c34)ccc2c1C(=O)NC1CC(C)(C)C1 ; Cc1oc2cc(Oc3ccnn4cc(C(=O)N5CC[C@@](C)(O)C5)c(C)c34)ccc2c1C(=O)NC1CCC1(C)C]

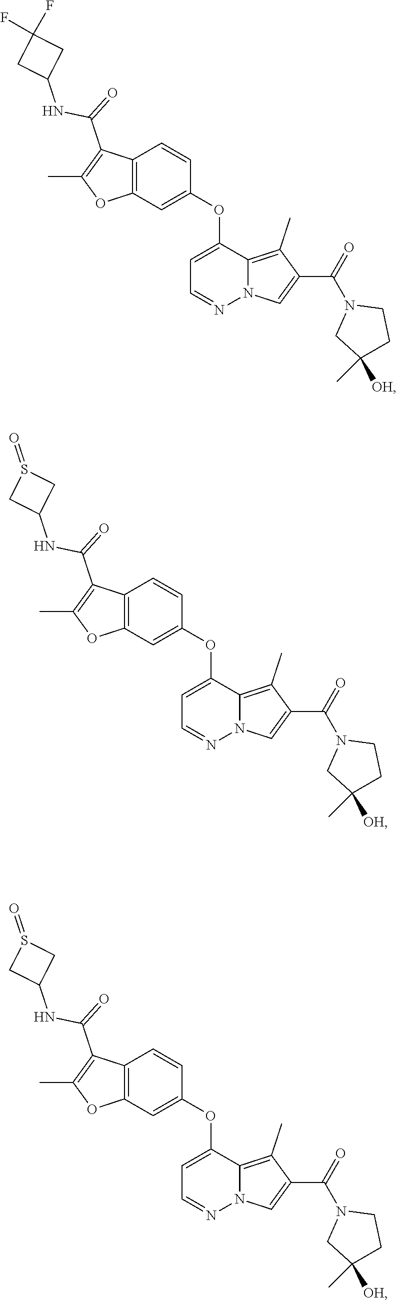 [SMILES: Cc1oc2cc(Oc3ccnn4cc(C(=O)N5CC[C@@](C)(O)C5)c(C)c34)ccc2c1C(=O)NC1CC(F)(F)C1 ; Cc1oc2cc(Oc3ccnn4cc(C(=O)N5CC[C@@](C)(O)C5)c(C)c34)ccc2c1C(=O)NC1CS(=O)C1 ; Cc1oc2cc(Oc3ccnn4cc(C(=O)N5CC[C@@](C)(O)C5)c(C)c34)ccc2c1C(=O)NC1CS(=O)C1]

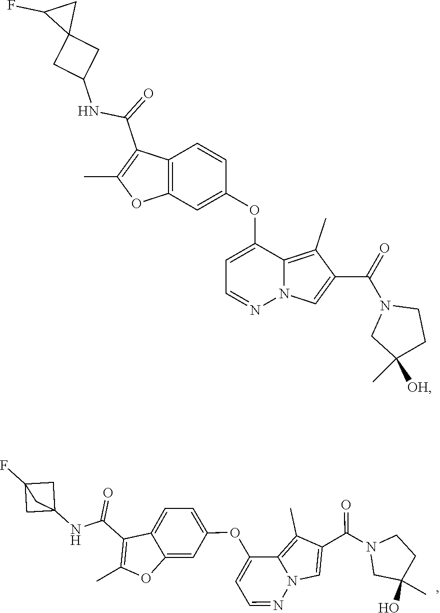 [SMILES: Cc1oc2cc(Oc3ccnn4cc(C(=O)N5CC[C@@](C)(O)C5)c(C)c34)ccc2c1C(=O)NC1CC2(C1)CC2F ; Cc1oc2cc(Oc3ccnn4cc(C(=O)N5CC[C@@](C)(O)C5)c(C)c34)ccc2c1C(=O)NC12CC(F)(C1)C2]

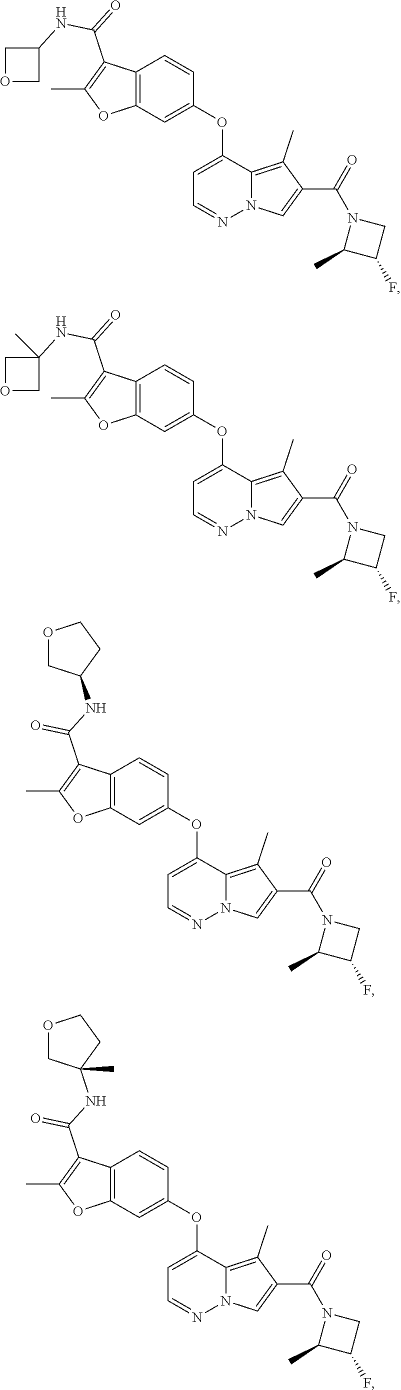 [SMILES: Cc1oc2cc(Oc3ccnn4cc(C(=O)N5C[C@H](F)[C@H]5C)c(C)c34)ccc2c1C(=O)NC1COC1 ; Cc1oc2cc(Oc3ccnn4cc(C(=O)N5C[C@H](F)[C@H]5C)c(C)c34)ccc2c1C(=O)NC1(C)COC1 ; Cc1oc2cc(Oc3ccnn4cc(C(=O)N5C[C@H](F)[C@H]5C)c(C)c34)ccc2c1C(=O)N[C@@H]1CCOC1 ; Cc1oc2cc(Oc3ccnn4cc(C(=O)N5C[C@H](F)[C@H]5C)c(C)c34)ccc2c1C(=O)N[C@@]1(C)CCOC1]

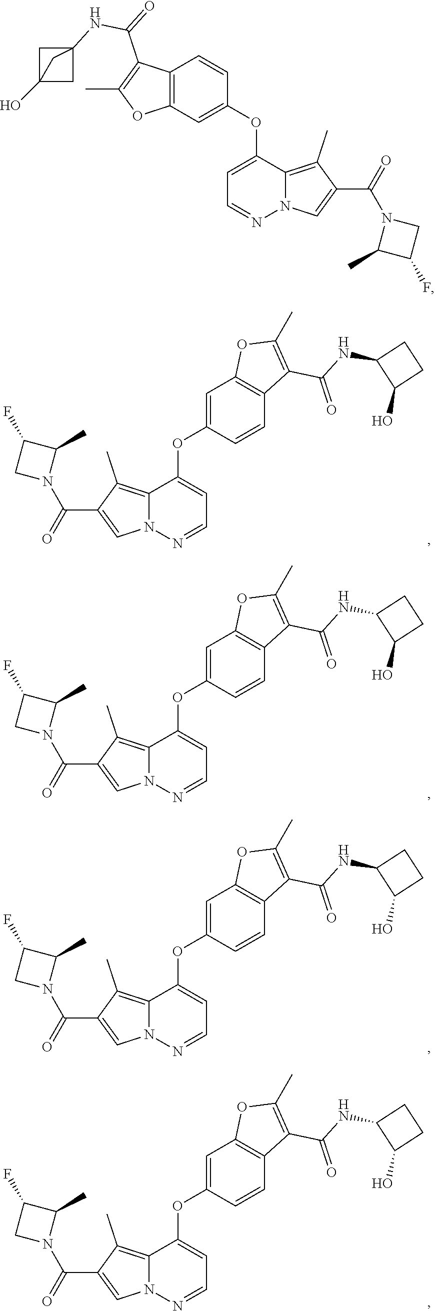 [SMILES: Cc1oc2cc(Oc3ccnn4cc(C(=O)N5C[C@H](F)[C@H]5C)c(C)c34)ccc2c1C(=O)NC12CC(O)(C1)C2 ; Cc1oc2cc(Oc3ccnn4cc(C(=O)N5C[C@H](F)[C@H]5C)c(C)c34)ccc2c1C(=O)N[C@H]1CC[C@H]1O ; Cc1oc2cc(Oc3ccnn4cc(C(=O)N5C[C@H](F)[C@H]5C)c(C)c34)ccc2c1C(=O)N[C@@H]1CC[C@H]1O ; Cc1oc2cc(Oc3ccnn4cc(C(=O)N5C[C@H](F)[C@H]5C)c(C)c34)ccc2c1C(=O)N[C@H]1CC[C@@H]1O ; Cc1oc2cc(Oc3ccnn4cc(C(=O)N5C[C@H](F)[C@H]5C)c(C)c34)ccc2c1C(=O)N[C@@H]1CC[C@@H]1O]

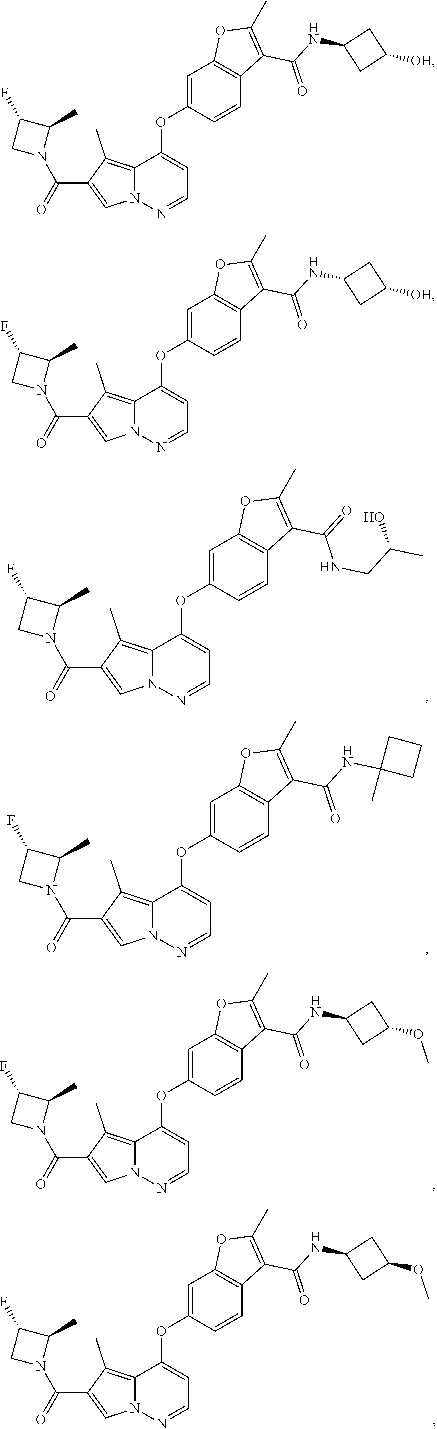 [SMILES: Cc1oc2cc(Oc3ccnn4cc(C(=O)N5C[C@H](F)[C@H]5C)c(C)c34)ccc2c1C(=O)N[C@H]1C[C@H](O)C1 ; Cc1oc2cc(Oc3ccnn4cc(C(=O)N5C[C@H](F)[C@H]5C)c(C)c34)ccc2c1C(=O)N[C@H]1C[C@@H](O)C1 ; Cc1oc2cc(Oc3ccnn4cc(C(=O)N5C[C@H](F)[C@H]5C)c(C)c34)ccc2c1C(=O)NC[C@@H](C)O ; Cc1oc2cc(Oc3ccnn4cc(C(=O)N5C[C@H](F)[C@H]5C)c(C)c34)ccc2c1C(=O)NC1(C)CCC1 ; CO[C@H]1C[C@H](NC(=O)c2c(C)oc3cc(Oc4ccnn5cc(C(=O)N6C[C@H](F)[C@H]6C)c(C)c45)ccc23)C1 ; CO[C@H]1C[C@@H](NC(=O)c2c(C)oc3cc(Oc4ccnn5cc(C(=O)N6C[C@H](F)[C@H]6C)c(C)c45)ccc23)C1]

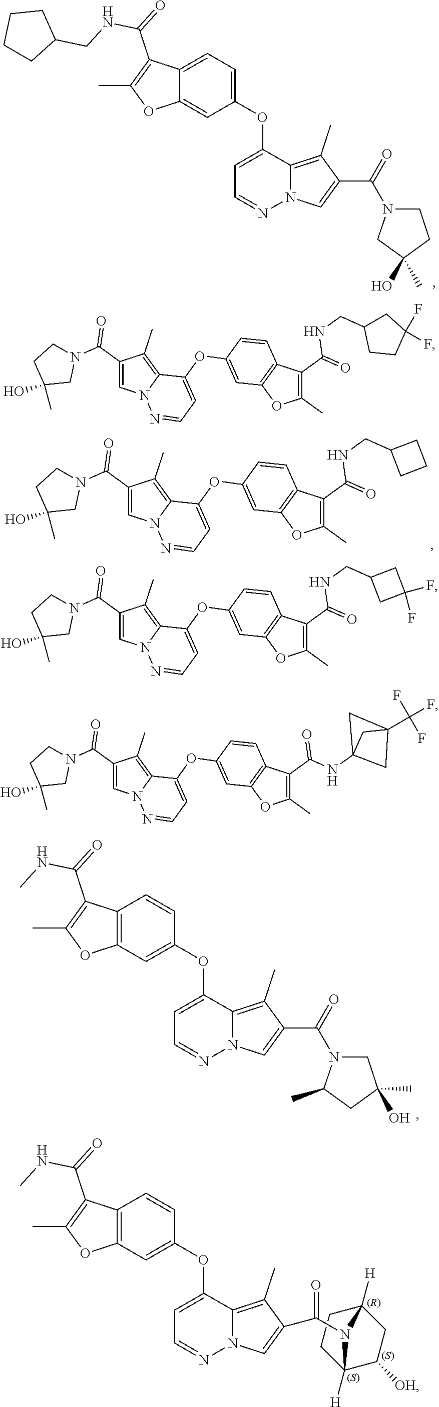

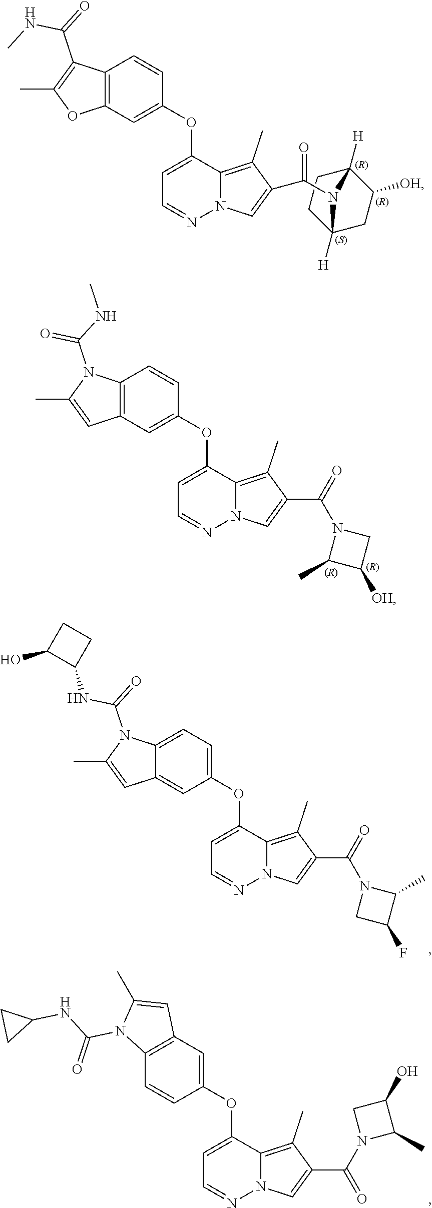

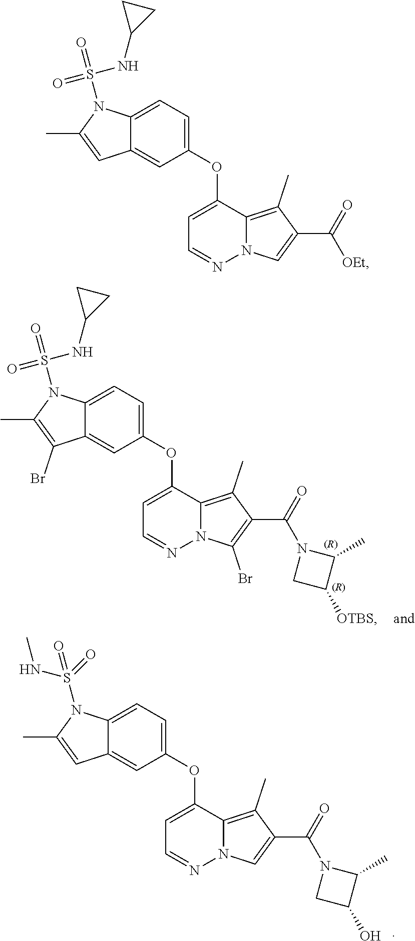

28. A compound of claim 1, or pharmaceutically acceptable salt thereof, selected from:
methyl 4-((2-methyl-3-(methylcarbamoyl)benzofuran-6-yl)oxy)pyrrolo[1,2-b]pyridazine-6-carboxylate,
ethyl 4-[(4-fluoro-2-methyl-1H-indol-5-yl)oxy]-5-methylpyrrolo[1,2-b]pyridazine-6-carboxylate,
ethyl 5-methyl-4-{[2-methyl-3-(methylcarbamoyl)-1-benzofuran-6-yl]oxy}pyrrolo[1,2-b]pyridazine-6-carboxylate,
N,2-dimethyl-6-((6-(pyrrolidine-1-carbonyl)pyrrolo[1,2-b]pyridazin-4-yl)oxy)benzofuran-3-carboxamide),
N,2-dimethyl-6-((5-methyl-6-(pyrrolidine-1-carbonyl)pyrrolo[1,2-b]pyridazin-4-yl)oxy)benzofuran-3-carboxamide,
(R)-6-((6-(3-hydroxy-3-methylpyrrolidine-1-carbonyl)-5-methylpyrrolo[1,2-b]pyridazin-4-yl)oxy)-N,2-dimethylbenzofuran-3-carboxamide,
5-methyl-N-(4-methyl-1,1-dioxidotetrahydro-2H-thiopyran-4-yl)-4-((2-methyl-3-(methylcarbamoyl)benzofuran-6-yl)oxy)pyrrolo[1,2-b]pyridazine-6-carboxamide,
6-((6-(4-(2-hydroxypropan-2-yl)-2-azabicyclo[2.1.1]hexane-2-carbonyl)-5-methylpyrrolo[1,2-b]pyridazin-4-yl)oxy)-N,2-dimethylbenzofuran-3-carboxamide,
6-({6-[(3R)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
6-({6-[(3S)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
6-{[6-(3-cyanoazetidine-1-carbonyl)-5-methylpyrrolo[1,2-b]pyridazin-4-yl]oxy}-N,2-dimethyl-1-benzofuran-3-carboxamide,
6-{[6-(3-methoxyazetidine-1-carbonyl)-5-methylpyrrolo[1,2-b]pyridazin-4-yl]oxy}-N,2-dimethyl-1-benzofuran-3-carboxamide,
5-methyl-N-(3-methyl-1,1-dioxo-1lambda˜6˜-thietan-3-yl)-4-{[2-methyl-3-(methylcarbamoyl)-1-benzofuran-6-yl]oxy}pyrrolo[1,2-b]pyridazine-6-carboxamide,
N,2-dimethyl-6-{[5-methyl-6-(7-oxo-2,6-diazaspiro[3.4]octane-2-carbonyl)pyrrolo[1,2-b]pyridazin-4-yl]oxy}-1-benzofuran-3-carboxamide,
6-{[6-(6-hydroxy-2-azaspiro[3.3]heptane-2-carbonyl)-5-methylpyrrolo[1,2-b]pyridazin-4-yl]oxy}-N,2-dimethyl-1-benzofuran-3-carboxamide,
6-({6-[4-(2-hydroxypropan-2-yl)-2-azabicyclo[2.1.1]hexane-2-carbonyl]-5-methylpyrrolo[1,2-b]pyridazin-4-yl}oxy)-N,2-dimethyl-1-benzofuran-3-carboxamide,
(rac)-6-{[6-(4,4-difluorohexahydrocyclopenta[c]pyrrole-2(1H)-carbonyl)-5-methylpyrrolo[1,2-b]pyridazin-4-yl]oxy}-N,2-dimethyl-1-benzofuran-3-carboxamide,
N,2-dimethyl-6-({5-methyl-6-[3-(1H-pyrazol-1-yl)azetidine-1-carbonyl]pyrrolo[1,2-b]pyridazin-4-yl}oxy)-1-benzofuran-3-carboxamide,
N,2-dimethyl-6-{[5-methyl-6-(5-oxa-2-azaspiro[3.4]octane-2-carbonyl)pyrrolo[1,2-b]pyridazin-4-yl]oxy}-1-benzofuran-3-carboxamide,
(rac)-6-{[6-(3-azabicyclo[3.1.0]hexane-3-carbonyl)-5-methylpyrrolo[1,2-b]pyridazin-4-yl]oxy}-N,2-dimethyl-1-benzofuran-3-carboxamide,
N,2-dimethyl-6-({5-methyl-6-[(1S,4S)-2-oxa-5-azabicyclo[2.2.1]heptane-5-carbonyl]pyrrolo[1,2-b]pyridazin-4-yl}oxy)-1-benzofuran-3-carboxamide,
(rac)-N,2-dimethyl-6-{[5-methyl-6-(6-oxo-1,7-diazaspiro[4.4]nonane-1-carbonyl)pyrrolo[1,2-b]pyridazin-4-yl]oxy}-1-benzofuran-3-carboxamide,
(rac)-6-{[6-(hexahydro-4H-furo[3,2-b]pyrrole-4-carbonyl)-5-methylpyrrolo[1,2-b]pyridazin-4-yl]oxy}-N,2-dimethyl-1-benzofuran-3-carboxamide,
(rac)-N,2-dimethyl-6-{[5-methyl-6-(1-oxa-7-azaspiro[4.4]nonane-7-carbonyl)pyrrolo[1,2-b]pyridazin-4-yl]oxy}-1-benzofuran-3-carboxamide,
N,2-dimethyl-6-1[(5-methyl-6-{3-[(1H-1,2,4-triazol-3-yl)methyl]azetidine-1-carbonyl}pyrrolo[1,2-b]pyridazin-4-yl)oxy]-1-benzofuran-3-carboxamide,
6-{[6-(3-hydroxy-3-methylazetidine-1-carbonyl)-5-methylpyrrolo[1,2-b]pyridazin-4-yl]oxy}-N,2-dimethyl-1-benzofuran-3-carboxamide,
N,2-dimethyl-6-{[5-methyl-6-(7-oxa-2-azaspiro[3.5]nonane-2-carbonyl)pyrrolo[1,2-b]pyridazin-4-yl]oxy}-1-benzofuran-3-carboxamide,
(rac)-N,2-dimethyl-6-({5-methyl-6-[3-(1H-pyrazol-1-yl)pyrrolidine-1-carbonyl]pyrrolo[1,2-b]pyridazin-4-yl}oxy)-1-benzofuran-3-carboxamide,
N-(3-methoxy-2,2-dimethylpropyl)-5-methyl-4-{[2-methyl-3-(methylcarbamoyl)-1-benzofuran-6-yl]oxy}pyrrolo[1,2-b]pyridazine-6-carboxamide,
(rac)-N-(1-cyclopropylethyl)-5-methyl-4-{[2-methyl-3-(methylcarbamoyl)-1-benzofuran-6-yl]oxy}pyrrolo[1,2-b]pyridazine-6-carboxamide,
N-[(1S,2R)-2-hydroxycyclohexyl]-N,5-dimethyl-4-{[2-methyl-3-(methylcarbamoyl)-1-benzofuran-6-yl]oxy}pyrrolo[1,2-b]pyridazine-6-carboxamide,
(rac)-N,5-dimethyl-4-{[2-methyl-3-(methylcarbamoyl)-1-benzofuran-6-yl]oxy}-N-[(1-methyl-5-oxopyrrolidin-2-yl)methyl]pyrrolo[1,2-b]pyridazine-6-carboxamide,
6-({6-[(2R,4R)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R)-2-(methoxymethyl)pyrrolidine-1-carbonyl]-5-methylpyrrolo[1,2-b]pyridazin-4-yl}oxy)-N,2-dimethyl-1-benzofuran-3-carboxamide,
6-{[6-(2,2-dimethylpyrrolidine-1-carbonyl)-5-methylpyrrolo[1,2-b]pyridazin-4-yl]oxy}-N,2-dimethyl-1-benzofuran-3-carboxamide,
(rac)-6-{[6-(2-azabicyclo[3.1.0]hexane-2-carbonyl)-5-methylpyrrolo[1,2-b]pyridazin-4-yl]oxy}-N,2-dimethyl-1-benzofuran-3-carboxamide,
N,2-dimethyl-6-({5-methyl-6-[(2R)-2-methylpyrrolidine-1-carbonyl]pyrrolo[1,2-b]pyridazin-4-yl}oxy)-1-benzofuran-3-carboxamide,
N,2-dimethyl-6-({5-methyl-6-[(2S)-2-methylpyrrolidine-1-carbonyl]pyrrolo[1,2-b]pyridazin-4-yl}oxy)-1-benzofuran-3-carboxamide,
N,2-dimethyl-6-({5-methyl-6-[(3aS,7aS)-octahydro-1H-indole-1-carbonyl]pyrrolo[1,2-b]pyridazin-4-yl}oxy)-1-benzofuran-3-carboxamide,
(rac)-6-({6-[2-(cyanomethyl)pyrrolidine-1-carbonyl]-5-methylpyrrolo[1,2-b]pyridazin-4-yl}oxy)-N,2-dimethyl-1-benzofuran-3-carboxamide,
6-({6-[(2R,5S)-2,5-dimethylpyrrolidine-1-carbonyl]-5-methylpyrrolo[1,2-b]pyridazin-4-yl}oxy)-N,2-dimethyl-1-benzofuran-3-carboxamide,
(rac)-6-{[6-(3-hydroxy-3-methylpiperidine-1-carbonyl)-5-methylpyrrolo[1,2-b]pyridazin-4-yl]oxy}-N,2-dimethyl-1-benzofuran-3-carboxamide,
(rac)-6-{[6-(3-hydroxypiperidine-1-carbonyl)-5-methylpyrrolo[1,2-b]pyridazin-4-yl]oxy}-N,2-dimethyl-1-benzofuran-3-carboxamide,
(rac)-6-{[6-(2-ethylpiperidine-1-carbonyl)-5-methylpyrrolo[1,2-b]pyridazin-4-yl]oxy}-N,2-dimethyl-1-benzofuran-3-carboxamide,
(trans)-6-{[6-(hexahydro-2H-pyrano[3,2-c]pyridine-6(5H)-carbonyl)-5-methylpyrrolo[1,2-b]pyridazin-4-yl]oxy}-N,2-dimethyl-1-benzofuran-3-carboxamide,
(cis)-6-{[6-(hexahydro-2H-pyrano[3,2-c]pyridine-6(5H)-carbonyl)-5-methylpyrrolo[1,2-b]pyridazin-4-yl]oxy}-N,2-dimethyl-1-benzofuran-3-carboxamide,
(rac)-N,2-dimethyl-6-{[5-methyl-6-(1-oxa-7-azaspiro[4.5]decane-7-carbonyl)pyrrolo[1,2-b]pyridazin-4-yl]oxy}-1-benzofuran-3-carboxamide,
(rac)-N,2-dimethyl-6-{[5-methyl-6-(1-oxo-2-oxa-7-azaspiro[4.5]decane-7-carbonyl)pyrrolo[1,2-b]pyridazin-4-yl]oxy}-1-benzofuran-3-carboxamide,
(rac)-6-{[6-(hexahydrofuro[3,2-b]pyridine-4(2H)-carbonyl)-5-methylpyrrolo[1,2-b]pyridazin-4-yl]oxy}-N,2-dimethyl-1-benzofuran-3-carboxamide,
(rac)-N,2-dimethyl-6-{[5-methyl-6-(2-methyloctahydro-4H-1,4-benzoxazine-4-carbonyl)pyrrolo[1,2-b]pyridazin-4-yl]oxy}-1-benzofuran-3-carboxamide,
N,2-dimethyl-6-({5-methyl-6-[(3S)-3-(propan-2-yl)morpholine-4-carbonyl]pyrrolo[1,2-b]pyridazin-4-yl}oxy)-1-benzofuran-3-carboxamide,
(rac)-6-{[6-(2-azabicyclo[2.2.1]heptane-2-carbonyl)-5-methylpyrrolo[1,2-b]pyridazin-4-yl]oxy}-N,2-dimethyl-1-benzofuran-3-carboxamide,
6-{[6-(azepane-1-carbonyl)-5-methylpyrrolo[1,2-b]pyridazin-4-yl]oxy}-N,2-dimethyl-1-benzofuran-3-carboxamide,
(rac)-6-{[6-(3-hydroxyazepane-1-carbonyl)-5-methylpyrrolo[1,2-b]pyridazin-4-yl]oxy}-N,2-dimethyl-1-benzofuran-3-carboxamide,
N,2-dimethyl-6-{[5-methyl-6-(1,4-oxazepane-4-carbonyl)pyrrolo[1,2-b]pyridazin-4-yl]oxy}-1-benzofuran-3-carboxamide,
(rac)-ethyl 3-(5-methyl-4-{[2-methyl-3-(methylcarbamoyl)-1-benzofuran-6-yl]oxy}pyrrolo[1,2-b]pyridazine-6-carbonyl)-3-azabicyclo[3.1.0]hexane-2-carboxylate,
N,2-dimethyl-6-({5-methyl-6-[(2S)-2-methylmorpholine-4-carbonyl]pyrrolo[1,2-b]pyridazin-4-yl}oxy)-1-benzofuran-3-carboxamide,
N,2-dimethyl-6-({5-methyl-6-[(2R)-2-methylmorpholine-4-carbonyl]pyrrolo[1,2-b]pyridazin-4-yl}oxy)-1-benzofuran-3-carboxamide,
N-[(1R,2R)-2-hydroxycyclohexyl]-N,5-dimethyl-4-{[2-methyl-3-(methylcarbamoyl)-1-benzofuran-6-yl]oxy}pyrrolo[1,2-b]pyridazine-6-carboxamide,
6-({6-[(2S)-2-(methoxymethyl)pyrrolidine-1-carbonyl]-5-methylpyrrolo[1,2-b]pyridazin-4-yl}oxy)-N,2-dimethyl-1-benzofuran-3-carboxamide,
6-({6-[(2S,3R)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R,3R)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R,3S)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2S,4S)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2S,4R)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-({6-[(2R,4S)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
N,2-dimethyl-6-({5-methyl-6-[(2S)-2-methylazetidine-1-carbonyl]pyrrolo[1,2-b]pyridazin-4-yl}oxy)-1-benzofuran-3-carboxamide,
N,2-dimethyl-6-({5-methyl-6-[(2R)-2-methylazetidine-1-carbonyl]pyrrolo[1,2-b]pyridazin-4-yl}oxy)-1-benzofuran-3-carboxamide,
6-({6-[(2R)-2-(methoxymethyl)azetidine-1-carbonyl]-5-methylpyrrolo[1,2-b]pyridazin-4-yl}oxy)-N,2-dimethyl-1-benzofuran-3-carboxamide,
6-({6-[(2S)-2-(methoxymethyl)azetidine-1-carbonyl]-5-methylpyrrolo[1,2-b]pyridazin-4-yl}oxy)-N,2-dimethyl-1-benzofuran-3-carboxamide,
6-({6-[(2R,3R)-3-fluoro-2-methylazetidine-1-carbonyl]-5-methylpyrrolo[1,2-b]pyridazin-4-yl}oxy)-N,2-dimethyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3R)-3-fluoro-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3S)-3-fluoro-2-methylazetidine-1-carbonyl]-5-methylpyrrolo[1,2-b]pyridazin-4-yl}oxy)-N,2-dimethyl-1-benzofuran-3-carboxamide,
7-fluoro-6-({6-[(2R,3R)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3S)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2S,3R)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2R,3S)-3-hydroxy-2-methylazetidine-1-carbonyl]-5-methylpyrrolo[1,2-b]pyridazin-4-yl}oxy)-N,2-dimethyl-1-benzofuran-3-carboxamide,
6-({6-[(2R,3S)-3-hydroxy-2-(trifluoromethyl)azetidine-1-carbonyl]-5-methylpyrrolo[1,2-b]pyridazin-4-yl}oxy)-N,2-dimethyl-1-benzofuran-3-carboxamide,
(rac)-6-{[6-(3,3-difluoro-2-methylazetidine-1-carbonyl)-5-methylpyrrolo[1,2-b]pyridazin-4-yl]oxy}-N,2-dimethyl-1-benzofuran-3-carboxamide,
6-({6-[(2S,3S)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
6-[(6-{(3S)-3-[acetyl(methyl)amino]pyrrolidine-1-carbonyl}-5-methylpyrrolo[1,2-b]pyridazin-4-yl)oxy]-N,2-dimethyl-1-benzofuran-3-carboxamide,
6-[(6-{(3R)-3-[acetyl(methyl)amino]pyrrolidine-1-carbonyl}-5-methylpyrrolo[1,2-b]pyridazin-4-yl)oxy]-N,2-dimethyl-1-benzofuran-3-carboxamide,
(rac)-6-[(6-{3-hydroxy-3-[(1H-1,2,3-triazol-1-yl)methyl]pyrrolidine-1-carbonyl}-5-methylpyrrolo[1,2-b]pyridazin-4-yl)oxy]-N,2-dimethyl-1-benzofuran-3-carboxamide,
(rac)-6-({6-[3-(difluoromethyl)-3-hydroxypyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(pyridin-3-yl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(methoxymethyl)pyrrolidine-1-carbonyl]-5-methylpyrrolo[1,2-b]pyridazin-4-yl}oxy)-N,2-dimethyl-1-benzofuran-3-carboxamide,
(rac)-N,2-dimethyl-6-{[5-methyl-6-(1-oxa-6-azaspiro[3.4]octane-6-carbonyl)pyrrolo[1,2-b]pyridazin-4-yl]oxy}-1-benzofuran-3-carboxamide,
(rac)-6-{[6-(3-ethyl-3-hydroxypyrrolidine-1-carbonyl)-5-methylpyrrolo[1,2-b]pyridazin-4-yl]oxy}-N,2-dimethyl-1-benzofuran-3-carboxamide,
6-{[6-(4-methoxy-2-azabicyclo[2.1.1]hexane-2-carbonyl)-5-methylpyrrolo[1,2-b]pyridazin-4-yl]oxy}-N,2-dimethyl-1-benzofuran-3-carboxamide,
(rac)-6-[(6-{3-hydroxy-3-[4-(trifluoromethyl)phenyl]pyrrolidine-1-carbonyl}-5-methylpyrrolo[1,2-b]pyridazin-4-yl)oxy]-N,2-dimethyl-1-benzofuran-3-carboxamide,
(rac)-6-({6-[3-hydroxy-3-(propan-2-yl)pyrrolidine-1-carbonyl]-5-methylpyrrolo[1,2-b]pyridazin-4-yl}oxy)-N,2-dimethyl-1-benzofuran-3-carboxamide,
6-({6-[(1R,5S)-1-methoxy-3-azabicyclo[3.2.0]heptane-3-carbonyl]-5-methylpyrrolo[1,2-b]pyridazin-4-yl}oxy)-N,2-dimethyl-1-benzofuran-3-carboxamide,
(rac)-6-({6-[3-(cyanomethyl)-3-hydroxypyrrolidine-1-carbonyl]-5-methylpyrrolo[1,2-b]pyridazin-4-yl}oxy)-N,2-dimethyl-1-benzofuran-3-carboxamide,
6-({6-[(1R,5S)-1-hydroxy-3-azabicyclo[3.2.0]heptane-3-carbonyl]-5-methylpyrrolo[1,2-b]pyridazin-4-yl}oxy)-N,2-dimethyl-1-benzofuran-3-carboxamide,
(rac)-6-({6-[3-methoxy-3-(trifluoromethyl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(1-methyl-1H-pyrazol-5-yl)pyrrolidine-1-carbonyl]-5-methylpyrrolo[1,2-b]pyridazin-4-yl}oxy)-N,2-dimethyl-1-benzofuran-3-carboxamide,
(rac)-N,2-dimethyl-6-({5-methyl-6-[1-(2,2,2-trifluoroethoxy)-3-azabicyclo[3.2.0]heptane-3-carbonyl]pyrrolo[1,2-b]pyridazin-4-yl}oxy)-1-benzofuran-3-carboxamide,
N,2-dimethyl-6-{[5-methyl-6-(1′H-spiro[3-oxabicyclo[3.1.0]hexane-2,3′-pyrrolidine]-1′-carbonyl)pyrrolo[1,2-b]pyridazin-4-yl]oxy}-1-benzofuran-3-carboxamide,
(rac)-6-[(6-{3-[(4-chlorophenyl)methyl]-3-hydroxypyrrolidine-1-carbonyl}-5-methylpyrrolo[1,2-b]pyridazin-4-yl)oxy]-N,2-dimethyl-1-benzofuran-3-carboxamide,
(rac)-6-({6-[3-(4-fluorophenyl)-3-hydroxypyrrolidine-1-carbonyl]-5-methylpyrrolo[1,2-b]pyridazin-4-yl}oxy)-N,2-dimethyl-1-benzofuran-3-carboxamide,
(rac)-6-{[6-(3-cyclopropyl-3-hydroxypyrrolidine-1-carbonyl)-5-methylpyrrolo[1,2-b]pyridazin-4-yl]oxy}-N,2-dimethyl-1-benzofuran-3-carboxamide,
(rac)-6-({6-[3-hydroxy-3-(2-methylpropyl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-hydroxy-3-(3-methyl-1,2,4-oxadiazol-5-yl)pyrrolidine-1-carbonyl]-5-methylpyrrolo[1,2-b]pyridazin-4-yl}oxy)-N,2-dimethyl-1-benzofuran-3-carboxamide,
(rac)-6-({6-[3-methoxy-3-(1-methyl-1H-imidazol-2-yl)pyrrolidine-1-carbonyl]-5-methylpyrrolo[1,2-b]pyridazin-4-yl}oxy)-N,2-dimethyl-1-benzofuran-3-carboxamide,
(rac)-6-{[6-(3-benzyl-3-hydroxypyrrolidine-1-carbonyl)-5-methylpyrrolo[1,2-b]pyridazin-4-yl]oxy}-N,2-dimethyl-1-benzofuran-3-carboxamide,
(rac)-6-({6-[3-hydroxy-3-(trifluoromethyl)pyrrolidine-1-carbonyl]-5-methylpyrrolo[1,2-b]pyridazin-4-yl}oxy)-N,2-dimethyl-1-benzofuran-3-carboxamide,
(rac)-6-{[6-(3,3-dimethyl-1-oxa-6-azaspiro[3.4]octane-6-carbonyl)-5-methylpyrrolo[1,2-b]pyridazin-4-yl]oxy}-N,2-dimethyl-1-benzofuran-3-carboxamide,
(rac)-6-[(6-{3-[(4-fluorophenyl)methyl]-3-hydroxypyrrolidine-1-carbonyl}-5-methylpyrrolo[1,2-b]pyridazin-4-yl)oxy]-N,2-dimethyl-1-benzofuran-3-carboxamide,
(rac)-6-[(6-{3-[(dimethylamino)methyl]-3-hydroxypyrrolidine-1-carbonyl}-5-methylpyrrolo[1,2-b]pyridazin-4-yl)oxy]-N,2-dimethyl-1-benzofuran-3-carboxamide,
6-({6-[(2S,4R)-4-hydroxy-2-(methoxymethyl)pyrrolidine-1-carbonyl]-5-methylpyrrolo[1,2-b]pyridazin-4-yl}oxy)-N,2-dimethyl-1-benzofuran-3-carboxamide,
N-cyclobutyl-6-{[6-(3-methoxyazetidine-1-carbonyl)-5-methylpyrrolo[1,2-b]pyridazin-4-yl]oxy}-2-methyl-1-benzofuran-3-carboxamide,
N-cyclobutyl-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-cyclobutyl-6-({6-[(3S)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-2-methyl-6-({5-methyl-6-[(2R)-2-methylpyrrolidine-1-carbonyl]pyrrolo[1,2-b]pyridazin-4-yl}oxy)-1-benzofuran-3-carboxamide,
N-(bicyclo[1.1.1]pentan-1-yl)-6-({6-[(2R,4R)-4-hydroxy-2-methylpyrrolidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S)-2-(methoxymethyl)pyrrolidine-1-carbonyl]-5-methylpyrrolo[1,2-b]pyridazin-4-yl}oxy)-2-methyl-1-benzofuran-3-carboxamide,
Trans, N-(3,3-difluoro-2-hydroxycyclohexyl)-5-methyl-4-((2-methyl-3-(methylcarbamoyl)benzofuran-6-yl)oxy)pyrrolo[1,2-b]pyridazine-6-carboxamide),
N-(bicyclo[1.1.1]pentan-1-yl)-2-methyl-6-({5-methyl-6-[(2S)-2-methylazetidine-1-carbonyl]pyrrolo[1,2-b]pyridazin-4-yl}oxy)-1-benzofuran-3-carboxamide,
N-(bicyclo[1.1.1]pentan-1-yl)-2-methyl-6-({5-methyl-6-[(2R)-2-methylazetidine-1-carbonyl]pyrrolo[1,2-b]pyridazin-4-yl}oxy)-1-benzofuran-3-carboxamide,
N-(bicyclo[1.1.1]pentan-1-yl)-6-({6-[(2R)-2-(methoxymethyl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S)-2-(methoxymethyl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3R)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3S)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3R)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3S)-3-fluoro-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(2R,3S)-3-hydroxy-2-(trifluoromethyl)azetidine-1-carbonyl]-5-methylpyrrolo[1,2-b]pyridazin-4-yl}oxy)-2-methyl-1-benzofuran-3-carboxamide,
(rac)-N-(bicyclo[1.1.1]pentan-1-yl)-6-{[6-(3,3-difluoro-2-methylazetidine-1-carbonyl)-5-methylpyrrolo[1,2-b]pyridazin-4-yl]oxy}-2-methyl-1-benzofuran-3-carboxamide,
N-(bicyclo[1.1.1]pentan-1-yl)-6-{[6-(6-hydroxy-2-azaspiro[3.3]heptane-2-carbonyl)-5-methylpyrrolo[1,2-b]pyridazin-4-yl]oxy}-2-methyl-1-benzofuran-3-carboxamide,
N-(bicyclo[1.1.1]pentan-1-yl)-6-({6-[3-(difluoromethyl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[3-(2-hydroxypropan-2-yl)azetidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-2-methyl-6-{[5-methyl-6-(7-oxo-2,6-diazaspiro[3.4]octane-2-carbonyl)pyrrolo[1,2-b]pyridazin-4-yl]oxy}-1-benzofuran-3-carboxamide,
6-({6-[3-(acetylamino)-3-methylazetidine-1-carbonyl]-5-methylpyrrolo[1,2-b]pyridazin-4-yl}oxy)-N-(bicyclo[1.1.1]pentan-1-yl)-2-methyl-1-benzofuran-3-carboxamide,
N-(bicyclo[1.1.1]pentan-1-yl)-2-methyl-6-{[5-methyl-6-(6-oxo-5-oxa-2,7-diazaspiro[3.4]octane-2-carbonyl)pyrrolo[1,2-b]pyridazin-4-yl]oxy}-1-benzofuran-3-carboxamide,
N-(bicyclo[1.1.1]pentan-1-yl)-2-methyl-6-({5-methyl-6-[3-(2-oxopyrrolidin-1-yl)azetidine-1-carbonyl]pyrrolo[1,2-b]pyridazin-4-yl}oxy)-1-benzofuran-3-carboxamide,
6-({6-[3-(acetylamino)azetidine-1-carbonyl]-5-methylpyrrolo[1,2-b]pyridazin-4-yl}oxy)-N-(bicyclo[1.1.1]pentan-1-yl)-2-methyl-1-benzofuran-3-carboxamide,
N-(bicyclo[1.1.1]pentan-1-yl)-6-{[6-(3-hydroxyazetidine-1-carbonyl)-5-methylpyrrolo[1,2-b]pyridazin-4-yl]oxy}-2-methyl-1-benzofuran-3-carboxamide,
N-(bicyclo[1.1.1]pentan-1-yl)-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-[(1S,2R)-2-fluorocyclobutyl]-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,4S)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,4R)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,3S)-3-hydroxy-2-methylpyrrolidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2R,3R)-3-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,4S)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,4R)-4-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3S)-3-hydroxy-2-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2R,3R)-3-hydroxy-2-methylpyrrolidine-1-carbonyl]-5-methylpyrrolo[1,2-b]pyridazin-4-yl}oxy)-2-methyl-1-benzofuran-3-carboxamide,
6-({6-[(3S)-3-hydroxy-3-methylpyrrolidine-1-carbonyl]-5-methylpyrrolo[1,2-b]pyridazin-4-yl}oxy)-N,2,7-trimethyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-N,2,7-trimethyl-1-benzofuran-3-carboxamide,
N-cyclobutyl-6-({6-[(3S)-3-hydroxy-3-methylpyrrolidine-1-carbonyl]-5-methylpyrrolo[1,2-b]pyridazin-4-yl}oxy)-2,7-dimethyl-1-benzofuran-3-carboxamide,
N-cyclobutyl-6-({6-[(3R)-3-hydroxy-3-methylpyrrolidine-1-carbonyl]-5-methylpyrrolo[1,2-b]pyridazin-4-yl}oxy)-2,7-dimethyl-1-benzofuran-3-carboxamide,
7-fluoro-6-({6-[(3S)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
7-fluoro-6-({6-[(3R)-3-hydroxy-3-methylpyrrolidine-1-carbonyl]-5-methylpyrrolo[1,2-b]pyridazin-4-yl}oxy)-N,2-dimethyl-1-benzofuran-3-carboxamide,
7-fluoro-6-({6-[(2R,4S)-4-hydroxy-2-methylpyrrolidine-1-carbonyl]-5-methylpyrrolo[1,2-b]pyridazin-4-yl}oxy)-N,2-dimethyl-1-benzofuran-3-carboxamide,
(R)-{4-[(4-fluoro-2-methyl-1H-indol-5-yl)oxy]-5-methylpyrrolo[1,2-b]pyridazin-6-yl}(3-hydroxypyrrolidin-1-yl)methanone,
(S)-{4-[(4-fluoro-2-methyl-1H-indol-5-yl)oxy]-5-methylpyrrolo[1,2-b]pyridazin-6-yl}(3-hydroxypyrrolidin-1-yl)methanone,
{4-[(4-fluoro-2-methyl-1H-indol-5-yl)oxy]-5-methylpyrrolo[1,2-b]pyridazin-6-yl}(3-methoxyazetidin-1-yl)methanone,
{4-[(4-fluoro-2-methyl-1H-indol-5-yl)oxy]-5-methylpyrrolo[1,2-b]pyridazin-6-yl}[(3S)-3-methoxypyrrolidin-1-yl]methanone,
{4-[(4-fluoro-2-methyl-1H-indol-5-yl)oxy]-5-methylpyrrolo[1,2-b]pyridazin-6-yl}[(3R)-3-methoxypyrrolidin-1-yl]methanone,
{4-[(4-fluoro-2-methyl-1H-indol-5-yl)oxy]-5-methylpyrrolo[1,2-b]pyridazin-6-yl}[(3S)-3-hydroxy-3-methylpyrrolidin-1-yl]methanone,
{4-[(4-fluoro-2-methyl-1H-indol-5-yl)oxy]-5-methylpyrrolo[1,2-b]pyridazin-6-yl}[(3R)-3-hydroxy-3-methylpyrrolidin-1-yl]methanone,
1-{4-[(4-fluoro-2-methyl-1H-indol-5-yl)oxy]-5-methylpyrrolo[1,2-b]pyridazine-6-carbonyl}azetidine-3-carbonitrile,
6-({6-[(3S)-3-hydroxy-3-methylpyrrolidine-1-carbonyl]-5-methylpyrrolo[1,2-b]pyridazin-4-yl}oxy)-N-methyl-1-benzothiophene-3-carboxamide,
6-({6-[(3R)-3-hydroxy-3-methylpyrrolidine-1-carbonyl]-5-methylpyrrolo[1,2-b]pyridazin-4-yl}oxy)-N-methyl-1-benzothiophene-3-carboxamide,
6-({6-[(3S)-3-hydroxy-3-methylpyrrolidine-1-carbonyl]-5-methylpyrrolo[1,2-b]pyridazin-4-yl}oxy)-N,2-dimethyl-1-benzothiophene-3-carboxamide,
6-({6-[(3R)-3-hydroxy-3-methylpyrrolidine-1-carbonyl]-5-methylpyrrolo[1,2-b]pyridazin-4-yl}oxy)-N,2-dimethyl-1-benzothiophene-3-carboxamide,
6-({6-[(2R,3R)-3-hydroxy-2-methylazetidine-1-carbonyl]-5-methylpyrrolo[1,2-b]pyridazin-4-yl}oxy)-N,2-dimethyl-1-benzothiophene-3-carboxamide,
6-({6-[(2R,4S)-4-hydroxy-2-methylpyrrolidine-1-carbonyl]-5-methylpyrrolo[1,2-b]pyridazin-4-yl}oxy)-N,2-dimethyl-1-benzothiophene-3-carboxamide,
6-({6-[(2R,3R)-3-hydroxy-2-methylpyrrolidine-1-carbonyl]-5-methylpyrrolo[1,2-b]pyridazin-4-yl}oxy)-N,2-dimethyl-1-benzothiophene-3-carboxamide,
N-ethyl-5-({6-[(2R,3R)-3-hydroxy-2-methylazetidine-1-carbonyl]-5-methylpyrrolo[1,2-b]pyridazin-4-yl}oxy)-2-methyl-1H-indole-1-carboxamide,
N-ethyl-5-({6-[(3S)-3-hydroxy-3-methylpyrrolidine-1-carbonyl]-5-methylpyrrolo[1,2-b]pyridazin-4-yl}oxy)-2-methyl-1H-indole-1-carboxamide,
N-ethyl-5-({6-[(3R)-3-hydroxy-3-methylpyrrolidine-1-carbonyl]-5-methylpyrrolo[1,2-b]pyridazin-4-yl}oxy)-2-methyl-1H-indole-1-carboxamide,
N-ethyl-5-((6-(4-hydroxy-2-azabicyclo[2.1.1]hexane-2-carbonyl)-5-methylpyrrolo[1,2-b]pyridazin-4-yl)oxy)-2-methyl-1H-indole-1-carboxamide,
(S)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
(R)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
4-fluoro-5-((6-(4-hydroxy-2-azabicyclo[2.1.1]hexane-2-carbonyl)-5-methylpyrrolo[1,2-b]pyridazin-4-yl)oxy)-N,2-dimethyl-1H-indole-1-carboxamide,
5-((6-((2S,3S)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
5-((6-((2R,3R)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
6-({6-[(2S)-2-(difluoromethyl)azetidine-1-carbonyl]-5-methylpyrrolo[1,2-b]pyridazin-4-yl}oxy)-N,2-dimethyl-1-benzofuran-3-carboxamide,
N,2-dimethyl-6-({5-methyl-6-[(2S)-2-(trifluoromethyl)azetidine-1-carbonyl]pyrrolo[1,2-b]pyridazin-4-yl}oxy)-1-benzofuran-3-carboxamide,
N,2-dimethyl-6-({5-methyl-6-[2-(propan-2-yl)azetidine-1-carbonyl]pyrrolo[1,2-b]pyridazin-4-yl}oxy)-1-benzofuran-3-carboxamide,
6-{[6-(2-cyclopropylazetidine-1-carbonyl)-5-methylpyrrolo[1,2-b]pyridazin-4-yl]oxy}-N,2-dimethyl-1-benzofuran-3-carboxamide,
6-({6-[(2R,4S)-2,4-dimethylazetidine-1-carbonyl]-5-methylpyrrolo[1,2-b]pyridazin-4-yl}oxy)-N,2-dimethyl-1-benzofuran-3-carboxamide,
6-({6-[(2R,4R)-2,4-dimethylazetidine-1-carbonyl]-5-methylpyrrolo[1,2-b]pyridazin-4-yl}oxy)-N,2-dimethyl-1-benzofuran-3-carboxamide,
6-({6-[(2S,4S)-2,4-dimethylazetidine-1-carbonyl]-5-methylpyrrolo[1,2-b]pyridazin-4-yl}oxy)-N,2-dimethyl-1-benzofuran-3-carboxamide,
6-{[6-(6-cyano-2-azaspiro[3.3]heptane-2-carbonyl)-5-methylpyrrolo[1,2-b]pyridazin-4-yl]oxy}-N,2-dimethyl-1-benzofuran-3-carboxamide,
6-{[6-(3,3-dimethylazetidine-1-carbonyl)-5-methylpyrrolo[1,2-b]pyridazin-4-yl]oxy}-N,2-dimethyl-1-benzofuran-3-carboxamide,
(rac)-6-[(6-{3-[(dimethylamino)methyl]-3-hydroxyazetidine-1-carbonyl}-5-methylpyrrolo[1,2-b]pyridazin-4-yl)oxy]-N,2-dimethyl-1-benzofuran-3-carboxamide,
(rac)-6-{[6-(3-ethyl-3-fluoroazetidine-1-carbonyl)-5-methylpyrrolo[1,2-b]pyridazin-4-yl]oxy}-N,2-dimethyl-1-benzofuran-3-carboxamide,
6-{[6-(5-azaspiro[2.3]hexane-5-carbonyl)-5-methylpyrrolo[1,2-b]pyridazin-4-yl]oxy}-N,2-dimethyl-1-benzofuran-3-carboxamide,
(rac)-6-{[6-(3-cyano-3-fluoroazetidine-1-carbonyl)-5-methylpyrrolo[1,2-b]pyridazin-4-yl]oxy}-N,2-dimethyl-1-benzofuran-3-carboxamide,
(rac)-6-{[6-(3-cyano-3-hydroxyazetidine-1-carbonyl)-5-methylpyrrolo[1,2-b]pyridazin-4-yl]oxy}-N,2-dimethyl-1-benzofuran-3-carboxamide,
6-({6-[3-(2-cyanopropan-2-yl)azetidine-1-carbonyl]-5-methylpyrrolo[1,2-b]pyridazin-4-yl}oxy)-N,2-dimethyl-1-benzofuran-3-carboxamide,
(rac)-6-{[6-(3-cyclopropyl-3-methylazetidine-1-carbonyl)-5-methylpyrrolo[1,2-b]pyridazin-4-yl]oxy}-N,2-dimethyl-1-benzofuran-3-carboxamide,
N,2-dimethyl-6-{[5-methyl-6-(1-methyl-2-azabicyclo[2.1.1]hexane-2-carbonyl)pyrrolo[1,2-b]pyridazin-4-yl]oxy}-1-benzofuran-3-carboxamide,
6-({6-[1-(hydroxymethyl)-2-azabicyclo[2.1.1]hexane-2-carbonyl]-5-methylpyrrolo[1,2-b]pyridazin-4-yl}oxy)-N,2-dimethyl-1-benzofuran-3-carboxamide,
6-{[6-(7-azabicyclo[2.2.1]heptane-7-carbonyl)-5-methylpyrrolo[1,2-b]pyridazin-4-yl]oxy}-N,2-dimethyl-1-benzofuran-3-carboxamide,
6-{[6-(6-azabicyclo[3.1.1]heptane-6-carbonyl)-5-methylpyrrolo[1,2-b]pyridazin-4-yl]oxy}-N,2-dimethyl-1-benzofuran-3-carboxamide,
N-(cyclopropylmethyl)-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,3S)-3-hydroxy-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-{[6-(3-hydroxyazetidine-1-carbonyl)-5-methylpyrrolo[1,2-b]pyridazin-4-yl]oxy}-2-methyl-1-benzofuran-3-carboxamide,
N-(cyclopropylmethyl)-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-(cyclopropylmethyl)-6-({6-[(2R,3S)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,3R)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,3S)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,3R)-3-fluoro-2-methylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2S,4S)-2,4-dimethylazetidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(2R,4R)-2,4-dimethylazetidine-1-carbonyl]-5-methylpyrrolo[1,2-b]pyridazin-4-yl}oxy)-2-methyl-1-benzofuran-3-carboxamide,
6-{[6-(3-cyano-2-methylazetidine-1-carbonyl)-5-methylpyrrolo[1,2-b]pyridazin-4-yl]oxy}-N-(cyclopropylmethyl)-2-methyl-1-benzofuran-3-carboxamide,
6-({6-[3-(acetylamino)azetidine-1-carbonyl]-5-methylpyrrolo[1,2-b]pyridazin-4-yl}oxy)-N-(cyclopropylmethyl)-2-methyl-1-benzofuran-3-carboxamide,
N-(bicyclo[1.1.1]pentan-1-yl)-6-{[6-(4-hydroxy-2-azabicyclo[2.1.1]hexane-2-carbonyl)-5-methylpyrrolo[1,2-b]pyridazin-4-yl]oxy}-2-methyl-1-benzofuran-3-carboxamide,
7-fluoro-6-{[6-(4-hydroxy-2-azabicyclo[2.1.1]hexane-2-carbonyl)-5-methylpyrrolo[1,2-b]pyridazin-4-yl]oxy}-N,2-dimethyl-1-benzofuran-3-carboxamide,
(R)-N,2-dimethyl-6-((5-methyl-6-(3-(N-methylacetamido)pyrrolidine-1-carbonyl)pyrrolo[1,2-b]pyridazin-4-yl)oxy)benzo[b]thiophene-3-carboxamide,
6-((6-(4-hydroxy-2-azabicyclo[2.1.1]hexane-2-carbonyl)-5-methylpyrrolo[1,2-b]pyridazin-4-yl)oxy)-N,2-dimethylbenzo[b]thiophene-3-carboxamide,
6-({6-[(2S,3S)-3-hydroxy-2-methylazetidine-1-carbonyl]-5-methylpyrrolo[1,2-b]pyridazin-4-yl}oxy)-N,3-dimethyl-1-benzofuran-2-carboxamide,
6-({6-[(2R,3R)-3-hydroxy-2-methylazetidine-1-carbonyl]-5-methylpyrrolo[1,2-b]pyridazin-4-yl}oxy)-N,3-dimethyl-1-benzofuran-2-carboxamide,
6-({6-[(2R,3S)-3-hydroxy-2-methylazetidine-1-carbonyl]-5-methylpyrrolo[1,2-b]pyridazin-4-yl}oxy)-N,3-dimethyl-1-benzofuran-2-carboxamide,
6-{[6-(3-hydroxyazetidine-1-carbonyl)-5-methylpyrrolo[1,2-b]pyridazin-4-yl]oxy}-N,3-dimethyl-1-benzofuran-2-carboxamide,
6-{[6-(3-hydroxy-3-methylazetidine-1-carbonyl)-5-methylpyrrolo[1,2-b]pyridazin-4-yl]oxy}-N,3-dimethyl-1-benzofuran-2-carboxamide,
6-({6-[(2S,3S)-3-hydroxy-2-methylazetidine-1-carbonyl]-5-methylpyrrolo[1,2-b]pyridazin-4-yl}oxy)-N-methyl-1-benzofuran-2-carboxamide,
6-({6-[(2R,3R)-3-hydroxy-2-methylazetidine-1-carbonyl]-5-methylpyrrolo[1,2-b]pyridazin-4-yl}oxy)-N-methyl-1-benzofuran-2-carboxamide,
6-({6-[(2R,3S)-3-hydroxy-2-methylazetidine-1-carbonyl]-5-methylpyrrolo[1,2-b]pyridazin-4-yl}oxy)-N-methyl-1-benzofuran-2-carboxamide,
6-{[6-(3-hydroxyazetidine-1-carbonyl)-5-methylpyrrolo[1,2-b]pyridazin-4-yl]oxy}-N-methyl-1-benzofuran-2-carboxamide,
6-{[6-(3-hydroxy-3-methylazetidine-1-carbonyl)-5-methylpyrrolo[1,2-b]pyridazin-4-yl]oxy}-N-methyl-1-benzofuran-2-carboxamide,
6-({6-[(2R,3S)-3-hydroxy-2-methylazetidine-1-carbonyl]-5-methylpyrrolo[1,2-b]pyridazin-4-yl}oxy)-N,3-dimethyl-1-benzothiophene-2-carboxamide,
6-({6-[(2S,3S)-3-hydroxy-2-methylazetidine-1-carbonyl]-5-methylpyrrolo[1,2-b]pyridazin-4-yl}oxy)-N,3-dimethyl-1-benzothiophene-2-carboxamide,
6-({6-[(2R,3R)-3-hydroxy-2-methylazetidine-1-carbonyl]-5-methylpyrrolo[1,2-b]pyridazin-4-yl}oxy)-N,3-dimethyl-1-benzothiophene-2-carboxamide,
6-{[6-(4-hydroxy-2-azabicyclo[2.1.1]hexane-2-carbonyl)-5-methylpyrrolo[1,2-b]pyridazin-4-yl]oxy}-N,3-dimethyl-1-benzothiophene-2-carboxamide,
6-{[6-(2-azabicyclo[2.1.1]hexane-2-carbonyl)-5-methylpyrrolo[1,2-b]pyridazin-4-yl]oxy}-N,3-dimethyl-1-benzothiophene-2-carboxamide,
6-{[6-(3-hydroxyazetidine-1-carbonyl)-5-methylpyrrolo[1,2-b]pyridazin-4-yl]oxy}-N,3-dimethyl-1-benzothiophene-2-carboxamide,
6-{[6-(3-hydroxy-3-methylazetidine-1-carbonyl)-5-methylpyrrolo[1,2-b]pyridazin-4-yl]oxy}-N,3-dimethyl-1-benzothiophene-2-carboxamide,
6-[(6-{(3S)-3-[acetyl(methyl)amino]pyrrolidine-1-carbonyl}-5-methylpyrrolo[1,2-b]pyridazin-4-yl)oxy]-N,3-dimethyl-1-benzothiophene-2-carboxamide,
6-[(6-{(3R)-3-[acetyl(methyl)amino]pyrrolidine-1-carbonyl}-5-methylpyrrolo[1,2-b]pyridazin-4-yl)oxy]-N,3-dimethyl-1-benzothiophene-2-carboxamide,
6-({6-[(3R)-3-hydroxy-3-methylpyrrolidine-1-carbonyl]-5-methylpyrrolo[1,2-b]pyridazin-4-yl}oxy)-N,3-dimethyl-1-benzothiophene-2-carboxamide,
6-({6-[(2R,3R)-3-hydroxy-2-methylpyrrolidine-1-carbonyl]-5-methylpyrrolo[1,2-b]pyridazin-4-yl}oxy)-N,3-dimethyl-1-benzothiophene-2-carboxamide,
5-({6-[(2R,3R)-3-hydroxy-2-methylazetidine-1-carbonyl]-5-methylpyrrolo[1,2-b]pyridazin-4-yl}oxy)-N,1-dimethyl-1H-benzimidazole-2-carboxamide,
6-((6-((1R,4R)-2-oxa-5-azabicyclo[2.2.1]heptane-5-carbonyl)-5-methylpyrrolo[1,2-b]pyridazin-4-yl)oxy)-7-fluoro-N,2-dimethylbenzofuran-3-carboxamide,
5-((6-(4-hydroxy-2-azabicyclo[2.1.1]hexane-2-carbonyl)-5-methylpyrrolo[1,2-b]pyridazin-4-yl)oxy)-N,2-dimethyl-1H-indole-1-carboxamide,
N-((1R,3R)-3-hydroxycyclobutyl)-N,5-dimethyl-4-((2-methyl-1-(methylcarbamoyl)-1H-indol-5-yl)oxy)pyrrolo[1,2-b]pyridazine-6-carboxamide,
N,2-dimethyl-5-((5-methyl-6-(7-methyl-1,7-diazaspiro[3.5]nonane-1-carbonyl)pyrrolo[1,2-b]pyridazin-4-yl)oxy)-1H-indole-1-carboxamide,
5-((6-(4-(2-hydroxypropan-2-yl)-2-azabicyclo[2.1.1]hexane-2-carbonyl)-5-methylpyrrolo[1,2-b]pyridazin-4-yl)oxy)-N,2-dimethyl-1H-indole-1-carboxamide,
N-ethyl-5-((6-((2R,3R)-3-hydroxy-2,3-dimethylazetidine-1-carbonyl)-5-methylpyrrolo[1,2-b]pyridazin-4-yl)oxy)-2-methyl-1H-indole-1-carboxamide,
N-ethyl-5-((6-((2R,3R)-3-fluoro-2-methylazetidine-1-carbonyl)-5-methylpyrrolo[1,2-b]pyridazin-4-yl)oxy)-2-methyl-1H-indole-1-carboxamide,
5-((6-((1R,5R)-2-oxa-6-azabicyclo[3.2.0]heptane-6-carbonyl)-5-methylpyrrolo[1,2-b]pyridazin-4-yl)oxy)-N-ethyl-2-methyl-1H-indole-1-carboxamide,
5-((6-((1R,5R)-2-oxa-6-azabicyclo[3.2.0]heptane-6-carbonyl)-5-methylpyrrolo[1,2-b]pyridazin-4-yl)oxy)-N,2-dimethyl-1H-indole-1-carboxamide,
(S)-N-ethyl-5-((6-(7-hydroxy-2-oxa-5-azaspiro[3.4]octane-5-carbonyl)-5-methylpyrrolo[1,2-b]pyridazin-4-yl)oxy)-2-methyl-1H-indole-1-carboxamide,
(cis)-N,2-dimethyl-6-((5-methyl-6-(octahydro-1H-indole-1-carbonyl)pyrrolo[1,2-b]pyridazin-4-yl)oxy)benzofuran-3-carboxamide,
(cis)-6-((6-(2,5-dimethylpyrrolidine-1-carbonyl)-5-methylpyrrolo[1,2-b]pyridazin-4-yl)oxy)-N,2-dimethylbenzofuran-3-carboxamide,
(trans)-N-(2-hydroxycyclohexyl)-N,5-dimethyl-4-((2-methyl-3-(methylcarbamoyl)benzofuran-6-yl)oxy)pyrrolo[1,2-b]pyridazine-6-carboxamide,
6-((6-((2R,3R or 2S,3S)-3-cyano-2-methylazetidine-1-carbonyl)-5-methylpyrrolo[1,2-b]pyridazin-4-yl)oxy)-N,2-dimethylbenzofuran-3-carboxamide,
(R)-6-((6-(3-hydroxy-2,2-dimethylazetidine-1-carbonyl)-5-methylpyrrolo[1,2-b]pyridazin-4-yl)oxy)-N,2-dimethylbenzofuran-3-carboxamide,
(S)-6-((6-(3-hydroxy-2,2-dimethylazetidine-1-carbonyl)-5-methylpyrrolo[1,2-b]pyridazin-4-yl)oxy)-N,2-dimethylbenzofuran-3-carboxamide,
6-({6-[(2S,3S or 2R,3R)-3-hydroxy-2,3-dimethylazetidine-1-carbonyl]-5-methylpyrrolo[1,2-b]pyridazin-4-yl}oxy)-N,2-dimethyl-1-benzofuran-3-carboxamide,
6-({6-[(2S,3S)-3-hydroxy-2,3-dimethylazetidine-1-carbonyl]-5-methylpyrrolo[1,2-b]pyridazin-4-yl}oxy)-N,2-dimethyl-1-benzofuran-3-carboxamide,
6-({6-[(2R,3R)-3-hydroxy-2,3-dimethylazetidine-1-carbonyl]-5-methylpyrrolo[1,2-b]pyridazin-4-yl}oxy)-N,2-dimethyl-1-benzofuran-3-carboxamide,
(R)-5-({6-[(2R,3R)-3-hydroxy-2-methylazetidine-1-carbonyl]-5-methylpyrrolo[1,2-b]pyridazin-4-yl}oxy)-N-methyl-2,3-dihydro-1H-indene-1-carboxamide,
(S)-5-({6-[(2R,3R)-3-hydroxy-2-methylazetidine-1-carbonyl]-5-methylpyrrolo[1,2-b]pyridazin-4-yl}oxy)-N-methyl-2,3-dihydro-1H-indene-1-carboxamide,
(S)-N,2-dimethyl-6-((5-methyl-6-(8-oxo-2,7-diazaspiro[4.4]nonane-2-carbonyl)pyrrolo[1,2-b]pyridazin-4-yl)oxy)benzofuran-3-carboxamide,
(R)-N,2-dimethyl-6-((5-methyl-6-(8-oxo-2,7-diazaspiro[4.4]nonane-2-carbonyl)pyrrolo[1,2-b]pyridazin-4-yl)oxy)benzofuran-3-carboxamide,
6-({6-[(2S,3S)-3-methoxy-2-methylazetidine-1-carbonyl]-5-methylpyrrolo[1,2-b]pyridazin-4-yl}oxy)-N,2-dimethyl-1-benzofuran-3-carboxamide,
1-{4-[(2,3-dihydro-1-benzofuran-6-yl)amino]-5-methylpyrrolo[1,2-b]pyridazine-6-carbonyl}azetidine-3-carbonitrile,
N-((1R,3R)-3-(cyanomethyl)cyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
N-((1S,3S)-3-fluorocyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
N-((1R,3R)-3-fluorocyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
N-[(cis)-2-fluorocyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-((1S,3S)-3-(cyanomethyl)cyclobutyl)-6-((6-((R)-3-hydroxy-3-methylpyrrolidine-1-carbonyl)-5-methylpyrrolo[1,2-b]pyridazin-4-yl)oxy)-2-methylbenzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[3.3]heptan-2-yl)-1-benzofuran-3-carboxamide,
6-((6-((R)-3-hydroxy-3-methylpyrrolidine-1-carbonyl)-5-methylpyrrolo[1,2-b]pyridazin-4-yl)oxy)-2-methyl-N-((1S,3S)-3-methylcyclobutyl)benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(trans)-2-methylcyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(cis)-2-methylcyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-((S)-spiro[3.3]heptan-1-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-((R)-spiro[3.3]heptan-1-yl)-1-benzofuran-3-carboxamide,
N-({R}2,2-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-({S}2,2-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-((6-((R)-3-hydroxy-3-methylpyrrolidine-1-carbonyl)-5-methylpyrrolo[1,2-b]pyridazin-4-yl)oxy)-2-methyl-N-((1S,3S)-1-oxidothietan-3-yl)benzofuran-3-carboxamide,
6-((6-((R)-3-hydroxy-3-methylpyrrolidine-1-carbonyl)-5-methylpyrrolo[1,2-b]pyridazin-4-yl)oxy)-2-methyl-N-((1R,3R)-1-oxidothietan-3-yl)benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-(oxetan-3-yl)-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-(3-methyloxetan-3-yl)-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-[(3R)-oxolan-3-yl]-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-[(3S)-3-methyloxolan-3-yl]-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(3-hydroxybicyclo[1.1.1]pentan-1-yl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-[(1S,2R)-2-hydroxycyclobutyl]-2-methyl-1-benzofuran-3-carboxamide 6-({6-[(2R,3S)-3-fluoro-2-methylazetidine-1-carbonyl]-5-methylpyrrolo[1,2-b]pyridazin-4-yl}oxy)-N-[(1R,2R)-2-hydroxycyclobutyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-[(1S,2S)-2-hydroxycyclobutyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-[(1R,2S)-2-hydroxycyclobutyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(trans-3-hydroxycyclobutyl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(cis-3-hydroxycyclobutyl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-[(2R)-2-hydroxypropyl]-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-2-methyl-N-(1-methylcyclobutyl)-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(trans-3-methoxycyclobutyl)-2-methyl-1-benzofuran-3-carboxamide,
6-({6-[(2R,3S)-3-fluoro-2-methylazetidine-1-carbonyl]-5-methylpyrrolo[1,2-b]pyridazin-4-yl}oxy)-N-(cis-3-methoxycyclobutyl)-2-methyl-1-benzofuran-3-carboxamide,
N-[((S)-3,3-difluorocyclopentyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[((R)-3,3-difluorocyclopentyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-((6-((1R,2R,4S)-2-hydroxy-2-methyl-7-azabicyclo[2.2.1]heptane-7-carbonyl)-5-methylpyrrolo[1,2-b]pyridazin-4-yl)oxy)-N,2-dimethylbenzofuran-3-carboxamide,
6-((6-((2R,4S or 2R,4R)-4-hydroxy-2,4-dimethylpyrrolidine-1-carbonyl)-5-methylpyrrolo[1,2-b]pyridazin-4-yl)oxy)-N,2-dimethylbenzofuran-3-carboxamide,
6-((6-((1S,2S,4R)-2-hydroxy-7-azabicyclo[2.2.1]heptane-7-carbonyl)-5-methylpyrrolo[1,2-b]pyridazin-4-yl)oxy)-N,2-dimethylbenzofuran-3-carboxamide,
6-((6-((1R,2R,4S)-2-hydroxy-7-azabicyclo[2.2.1]heptane-7-carbonyl)-5-methylpyrrolo[1,2-b]pyridazin-4-yl)oxy)-N,2-dimethylbenzofuran-3-carboxamide,
5-((6-((2R,3S)-3-fluoro-2-methylazetidine-1-carbonyl)-5-methylpyrrolo[1,2-b]pyridazin-4-yl)oxy)-N-((1S,2S)-2-hydroxycyclobutyl)-2-methyl-1H-indole-1-carboxamide,
N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-carboxamide,
5-({6-[(2R,3R)-3-hydroxy-2-methylazetidine-1-carbonyl]-5-methylpyrrolo[1,2-b]pyridazin-4-yl}oxy)-N,2-dimethyl-1H-indole-1-sulfonamide,
7-fluoro-6-({6-[(2R,3R)-3-hydroxy-2-methylpyrrolidine-1-carbonyl]-5-methylpyrrolo[1,2-b]pyridazin-4-yl}oxy)-N,2-dimethyl-1-benzofuran-3-carboxamide,
N-[(1R,2S)-2-fluorocyclobutyl]-6-({6-[(2R,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2S,3R)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(2S,3S)-3-hydroxy-2-methylazetidine-1-carbonyl]-5-methylpyrrolo[1,2-b]pyridazin-4-yl}oxy)-2-methyl-1-benzofuran-3-carboxamide,
N-[(1S,2R)-2-fluorocyclobutyl]-6-{[6-(3-hydroxyazetidine-1-carbonyl)-5-methylpyrrolo[1,2-b]pyridazin-4-yl]oxy}-2-methyl-1-benzofuran-3-carboxamide,
N-[(1R,2S)-2-fluorocyclobutyl]-6-{[6-(3-hydroxyazetidine-1-carbonyl)-5-methylpyrrolo[1,2-b]pyridazin-4-yl]oxy}-2-methyl-1-benzofuran-3-carboxamide,
N-[(1S,2R)-2-fluorocyclobutyl]-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-[(1R,2S)-2-fluorocyclobutyl]-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
N-[(1R,2S)-2-fluorocyclobutyl]-6-{[6-(3-hydroxy-3-methylazetidine-1-carbonyl)-5-methylpyrrolo[1,2-b]pyridazin-4-yl]oxy}-2-methyl-1-benzofuran-3-carboxamide,
Cis,6-((6-(3-amino-2-methylazetidine-1-carbonyl)-5-methylpyrrolo[1,2-b]pyridazin-4-yl)oxy)-N,2-dimethylbenzofuran-3-carboxamide,
Trans,N-(3,3-difluoro-2-hydroxycyclohexyl)-5-methyl-4-((2-methyl-3-(methylcarbamoyl)benzofuran-6-yl)oxy)pyrrolo[1,2-b]pyridazine-6-carboxamide),
1-{4-[(2,3-dihydro-1-benzofuran-6-yl)amino]-5-methylpyrrolo[1,2-b]pyridazine-6-carbonyl}azetidine-3-carbonitrile,
(N,2-dimethyl-6-((5-methyl-6-(8-oxo-2,7-diazaspiro[4.4]nonane-2-carbonyl)pyrrolo[1,2-b]pyridazin-4-yl)oxy)benzofuran-3-carboxamide),
(R)-6-{[6-(3,3-difluoro-4-hydroxypiperidine-1-carbonyl)-5-methylpyrrolo[1,2-b]pyridazin-4-yl]oxy}-N,2-dimethyl-1-benzofuran-3-carboxamide,
(S)-6-{[6-(3,3-difluoro-4-hydroxypiperidine-1-carbonyl)-5-methylpyrrolo[1,2-b]pyridazin-4-yl]oxy}-N,2-dimethyl-1-benzofuran-3-carboxamide,
(R)-6-{[6-(3-methoxy-3-methylpyrrolidine-1-carbonyl)-5-methylpyrrolo[1,2-b]pyridazin-4-yl]oxy}-N,2-dimethyl-1-benzofuran-3-carboxamide,
(S)-6-{[6-(3-methoxy-3-methylpyrrolidine-1-carbonyl)-5-methylpyrrolo[1,2-b]pyridazin-4-yl]oxy}-N,2-dimethyl-1-benzofuran-3-carboxamide,
1-(4-((1H-indol-5-yl)oxy)-5-methylpyrrolo[1,2-b]pyridazine-6-carbonyl)azetidine-3-carbonitrile,
6-((6-(3-cyanoazetidine-1-carbonyl)-5-methylpyrrolo[1,2-b]pyridazin-4-yl)oxy)-N-cyclopropyl-2-methylbenzofuran-3-carboxamide,
6-{[6-(3-cyanoazetidine-1-carbonyl)-5-methylpyrrolo[1,2-b]pyridazin-4-yl]oxy}-N-cyclobutyl-2-methyl-1-benzofuran-3-carboxamide,
1-{4-[(2,3-dimethyl-1-benzofuran-6-yl)oxy]-5-methylpyrrolo[1,2-b]pyridazine-6-carbonyl}azetidine-3-carbonitrile,
6-{[6-(3-cyanoazetidine-1-carbonyl)-5-methylpyrrolo[1,2-b]pyridazin-4-yl]oxy}-N,2-dimethyl-1-benzothiophene-3-carboxamide,
1-{5-methyl-4-[(2-methyl-1H-indol-5-yl)oxy]pyrrolo[1,2-b]pyridazine-6-carbonyl}azetidine-3-carbonitrile,
1-{4-[(1H-indol-5-yl)oxy]-5-methylpyrrolo[1,2-b]pyridazine-6-carbonyl}azetidine-3-carbonitrile,
1-{4-[(1-benzofuran-7-yl)oxy]-5-methylpyrrolo[1,2-b]pyridazine-6-carbonyl}azetidine-3-carbonitrile,
1-{4-[(1-benzothiophen-5-yl)oxy]-5-methylpyrrolo[1,2-b]pyridazine-6-carbonyl}azetidine-3-carbonitrile,
1-{4-[(1-benzothiophen-6-yl)oxy]-5-methylpyrrolo[1,2-b]pyridazine-6-carbonyl}azetidine-3-carbonitrile,
ethyl (5-{[6-(3-cyanoazetidine-1-carbonyl)-5-methylpyrrolo[1,2-b]pyridazin-4-yl]oxy}-2,3-dihydro-1H-inden-2-yl)acetate,
1-{5-methyl-4-[(3-methyl-1-benzofuran-6-yl)oxy]pyrrolo[1,2-b]pyridazine-6-carbonyl}azetidine-3-carbonitrile,
1-{4-[(1-benzofuran-6-yl)oxy]-5-methylpyrrolo[1,2-b]pyridazine-6-carbonyl}azetidine-3-carbonitrile,
1-{5-methyl-4-[(3-methyl-1-benzothiophen-6-yl)oxy]pyrrolo[1,2-b]pyridazine-6-carbonyl}azetidine-3-carbonitrile,
1-{4-[(1H-indol-6-yl)oxy]-5-methylpyrrolo[1,2-b]pyridazine-6-carbonyl}azetidine-3-carbonitrile,
1-{4-[(2,3-dihydro-1-benzofuran-6-yl)oxy]-5-methylpyrrolo[1,2-b]pyridazine-6-carbonyl}azetidine-3-carbonitrile,
1-{4-[(1-benzofuran-5-yl)oxy]-5-methylpyrrolo[1,2-b]pyridazine-6-carbonyl}azetidine-3-carbonitrile,
methyl 6-{[6-(3-cyanoazetidine-1-carbonyl)-5-methylpyrrolo[1,2-b]pyridazin-4-yl]oxy}-2,3-dihydro-1-benzofuran-2-carboxylate,
N-benzyl-5-{[6-(3-cyanoazetidine-1-carbonyl)-5-methylpyrrolo[1,2-b]pyridazin-4-yl]oxy}-2-methyl-1-benzofuran-3-carboxamide,
1-{4-[(2,3-dimethyl-1H-indol-5-yl)oxy]-5-methylpyrrolo[1,2-b]pyridazine-6-carbonyl}azetidine-3-carbonitrile,
ethyl 5-{[6-(3-cyanoazetidine-1-carbonyl)-5-methylpyrrolo[1,2-b]pyridazin-4-yl]oxy}-2-methyl-1-benzofuran-3-carboxylate,
1-{4-[(2,3-dihydro-1-benzothiophen-6-yl)oxy]-5-methylpyrrolo[1,2-b]pyridazine-6-carbonyl}azetidine-3-carbonitrile,
methyl 7-{[6-(3-cyanoazetidine-1-carbonyl)-5-methylpyrrolo[1,2-b]pyridazin-4-yl]oxy}-1-benzofuran-4-carboxylate,
5-{[6-(3-cyanoazetidine-1-carbonyl)-5-methylpyrrolo[1,2-b]pyridazin-4-yl]oxy}-N,2-dimethyl-1-benzofuran-3-carboxamide,
methyl 6-{[6-(3-cyanoazetidine-1-carbonyl)-5-methylpyrrolo[1,2-b]pyridazin-4-yl]oxy}-1H-indole-3-carboxylate,
[(3R)-3-hydroxy-3-methylpyrrolidin-1-yl]{5-methyl-4-[(2-methyl-1H-indol-6-yl)oxy]pyrrolo[1,2-b]pyridazin-6-yl}methanone,
N-ethyl-5-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-ethyl-5-((6-(4-hydroxy-2-azabicyclo[2.1.1]hexane-2-carbonyl)-5-methylpyrrolo[1,2-b]pyridazin-4-yl)oxy)-2-methyl-1H-indole-1-carboxamide,
(S)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
(R)-5-((6-(3-hydroxy-3-methylpyrrolidine-1-carbonyl)-5-methylpyrrolo[1,2-b]pyridazin-4-yl)oxy)-N,2-dimethyl-1H-indole-1-carboxamide,
4-fluoro-5-((6-(4-hydroxy-2-azabicyclo[2.1.1]hexane-2-carbonyl)-5-methylpyrrolo[1,2-b]pyridazin-4-yl)oxy)-N,2-dimethyl-1H-indole-1-carboxamide,
5-((6-((2S,3S)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
5-((6-((2R,3R)-3-hydroxy-2,3-dimethylazetidine-1-carbonyl)-5-methylpyrrolo[1,2-b]pyridazin-4-yl)oxy)-N,2-dimethyl-1H-indole-1-carboxamide,
1-{5-methyl-4-[(2-methyl-1-benzofuran-5-yl)amino]pyrrolo[1,2-b]pyridazine-6-carbonyl}azetidine-3-carbonitrile,
1-{4-[(1-benzothiophen-6-yl)amino]-5-methylpyrrolo[1,2-b]pyridazine-6-carbonyl}azetidine-3-carbonitrile,
1-{4-[(1-benzothiophen-5-yl)amino]-5-methylpyrrolo[1,2-b]pyridazine-6-carbonyl}azetidine-3-carbonitrile,
1-{4-[(2,3-dihydro-1-benzofuran-6-yl)amino]-5-methylpyrrolo[1,2-b]pyridazine-6-carbonyl}azetidine-3-carbonitrile,
1-{4-[(1-benzofuran-4-yl)amino]-5-methylpyrrolo[1,2-b]pyridazine-6-carbonyl}azetidine-3-carbonitrile,
N-benzyl-6-{[6-(3-cyanoazetidine-1-carbonyl)-5-methylpyrrolo[1,2-b]pyridazin-4-yl]oxy}-2-methyl-1-benzofuran-3-carboxamide,
(R)-N-(cyclopentylmethyl)-6-((6-(3-hydroxy-3-methylpyrrolidine-1-carbonyl)-5-methylpyrrolo[1,2-b]pyridazin-4-yl)oxy)-2-methylbenzofuran-3-carboxamide,
6-{[6-(3-cyanoazetidine-1-carbonyl)-5-methylpyrrolo[1,2-b]pyridazin-4-yl]oxy}-N-(cyclopropylmethyl)-2-methyl-1-benzofuran-3-carboxamide,
N-(bicyclo[1.1.1]pentan-1-yl)-6-{[6-(3-cyanoazetidine-1-carbonyl)-5-methylpyrrolo[1,2-b]pyridazin-4-yl]oxy}-2-methyl-1-benzofuran-3-carboxamide,
1-(4-{[3-(3-fluoroazetidine-1-carbonyl)-2-methyl-1-benzofuran-6-yl]oxy}-5-methylpyrrolo[1,2-b]pyridazine-6-carbonyl)azetidine-3-carbonitrile,
N-benzyl-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(cyclopropylmethyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(bicyclo[1.1.1]pentan-1-yl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[trans-3-(cyanomethyl)cyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(3-cyanocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(cis-3-fluorocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[(1R,2S)-2-fluorocyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[cis-3-(cyanomethyl)cyclobutyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[3.3]heptan-2-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(cis-3-methylcyclobutyl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(1R,2R)-2-methylcyclobutyl]-1-benzofuran-3-carboxamide,
N-(trans-3-fluorocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[3-(propan-2-yl)cyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[3.3]heptan-1-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[3-(trifluoromethyl)cyclobutyl]-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(spiro[2.3]hexan-5-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(1-oxaspiro[3.3]heptan-6-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(trans-3-methylcyclobutyl)-1-benzofuran-3-carboxamide,
N-(3,3-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(2,2-dimethylcyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(3,3-difluorocyclobutyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(1-oxo-1lambda˜4˜-thietan-3-yl)-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-[(1S,2R)-2-methylcyclobutyl]-1-benzofuran-3-carboxamide,
N-(1-fluorospiro[2.3]hexan-5-yl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-({6-[(3R)-3-hydroxy-3-methylpyrrolidine-1-carbonyl]-5-methylpyrrolo[1,2-b]pyridazin-4-yl}oxy)-2-methyl-N-(1-oxo-1lambda˜4˜-thietan-3-yl)-1-benzofuran-3-carboxamide,
N-[(3,3-difluorocyclopentyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-(cyclobutylmethyl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
N-[(3,3-difluorocyclobutyl)methyl]-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide,
6-((6-((1S,2S,4R)-2-hydroxy-2-methyl-7-azabicyclo[2.2.1]heptane-7-carbonyl)-5-methylpyrrolo[1,2-b]pyridazin-4-yl)oxy)-N,2-dimethylbenzofuran-3-carboxamide,
5-((6-((2R,3R)-3-hydroxy-2-methylazetidine-1-carbonyl)-5-methylpyrrolo[1,2-b]pyridazin-4-yl)oxy)-N,2-dimethyl-1H-indole-1-carboxamide,
ethyl 4-((1-(N-cyclopropylsulfamoyl)-2-methyl-1H-indol-5-yl)oxy)-5-methylpyrrolo[1,2-b]pyridazine-6-carboxylate,
N-cyclopropyl-5-((6-((2R,3R)-3-hydroxy-2-methylazetidine-1-carbonyl)-5-methylpyrrolo[1,2-b]pyridazin-4-yl)oxy)-2-methyl-1H-indole-1-sulfonamide,
6-({6-[(3R)-3-hydroxy-3-methylpyrrolidine-1-carbonyl]-5-methylpyrrolo[1,2-b]pyridazin-4-yl}oxy)-2-methyl-N-[3-(trifluoromethyl)bicyclo[1.1.1]pentan-1-yl]-1-benzofuran-3-carboxamide, or
N-(3-fluorobicyclo[1.1.1]pentan-1-yl)-6-({6-[(3R)-3-hydroxy-3-methylpyrrolidine-1-carbonyl]-5-methylpyrrolo[1,2-b]pyridazin-4-yl}oxy)-2-methyl-1-benzofuran-3-carboxamide.

29. A composition comprising a pharmaceutically acceptable carrier and a compound according to claim 1, or a pharmaceutically acceptable salt thereof.

30. A method for treating cancer in a patient in need thereof, comprising administering to the patient a therapeutically effective amount of a compound of claim 1, or a pharmaceutically acceptable salt thereof.
